(19) United States
(12) Patent Application Publication
(10) Pub. No.: US 2024/0238265 A1
(43) Pub. Date: Jul. 18, 2024

(54) ADAMANTANE AMIDES AND THIOAMIDES FOR THE TREATMENT OF EBOLAVIRUS INFECTION

(71) Applicant: Arisan Therapeutics Inc., Carlsbad, CA (US)

(72) Inventors: Eric Brown, Santee, CA (US); Vidyasagar Reddy Gantla, San Diego, CA (US); Nadezda V. Sokolova, San Diego, CA (US); Gregory Henkel, Carlsbad, CA (US); Kenneth McCormack, Oceanside, CA (US); Craig Robert Johnson, San Marcos, CA (US)

(73) Assignee: Arisan Therapeutics Inc., Carlsbad, CA (US)

(21) Appl. No.: 17/925,579

(22) PCT Filed: Jun. 7, 2021
(86) PCT No.: PCT/US21/36251

§ 371 Date: Nov. 15, 2022

Related U.S. Application Data
(60) Provisional application No. 63/037,495, filed on Jun. 10, 2020.

(51) Int. Cl. A61K 31/445; A61K 31/165; A61K 31/18; A61K 31/439; A61K 45/06; A61P 31/14

(52) U.S. Cl. CPC A61K 31/445; A61K 31/165; A61K 31/18; A61K 31/439; A61K 45/06; A61P 31/14

(57) ABSTRACT

Compounds of Structural Formulae I, II, III, IV, V, VI, VII, and VIII were developed for the treatment of Ebolavirus infection, wherein, R¹, R², R⁸, X, Y, Q, W, and NR^{3a}R^{3b} are defined in the specification.

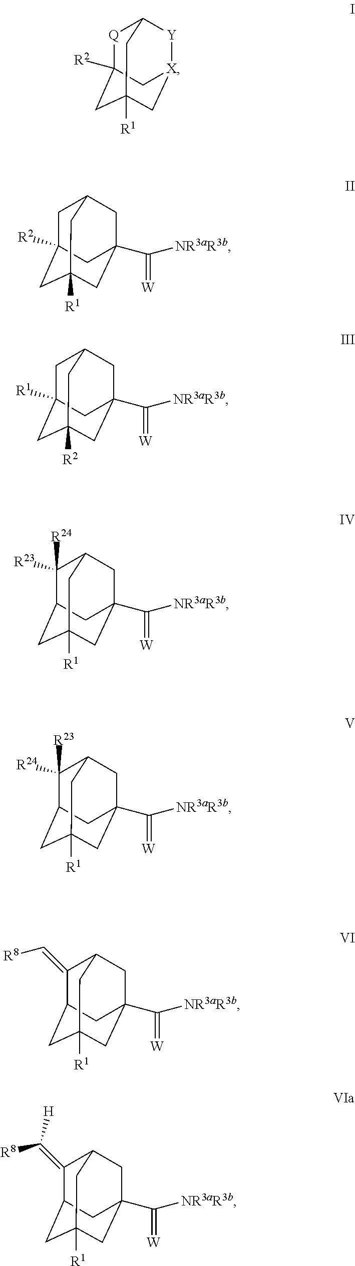

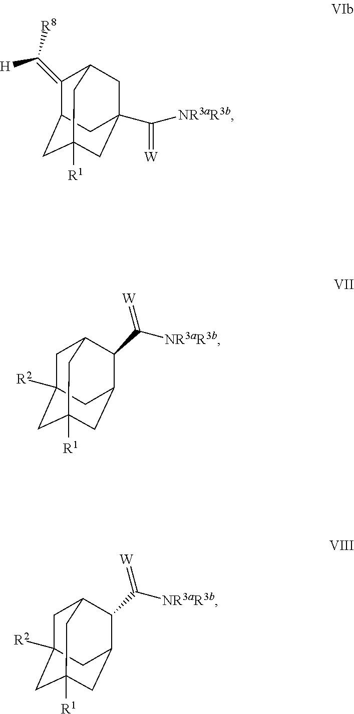

### CROSS REFERENCES TO RELATED APPLICATIONS

This patent application is a continuation-in-part of and claims the benefit of priority to U.S. Provisional Patent Application Ser. No. 63/037,495, filed Jun. 10, 2020 and is a continuation-in-part of PCT application PCT/US2021/036251, filed Jun. 7, 2021 both references are herein incorporated by reference in their entirety and for all purposes.

### GOVERNMENT SUPPORT

This invention was made with government support under R43 AI138878 awarded by U.S. National Institutes of Health. The government has certain rights in the invention.

### REFERENCE TO A SEQUENCE LISTING, A TABLE, OR A COMPUTER PROGRAM LISTING APPENDIX SUBMITTED ON A COMPACT DISK

NOT APPLICABLE

### FIELD OF THE INVENTION

The present invention relates to methods of inhibiting infection by viruses of the Filoviridae family (filoviruses) in humans, other mammals, or in cell culture, to treating infection by filoviruses, to methods of inhibiting the replication of filoviruses, to methods of reducing the amount of filoviruses, and to compositions that can be employed for such methods. These methods, applications, and compositions apply not only to Filoviridae viruses but also to any virus, whether naturally emerging or engineered, whose cell entry properties are determined by filovirus glycoproteins.

### BACKGROUND OF THE INVENTION

The invention relates to the use of compounds for the treatment and/or prophylaxis of infection of humans or other mammals by one or more of a number of enveloped viruses of the Filoviridae family (filoviruses) or any other native or engineered enveloped virus utilizing filovirus glycoproteins to mediate cell entry. Enveloped viruses are comprised of an outer host-derived lipid membrane and an inner nucleoprotein core, which contains the viral genetic material (whether RNA or DNA). Virus-cell fusion is the means by which all enveloped viruses enter cells and initiate disease-causing cycles of replication. In all cases virus-cell fusion is executed by one or more viral surface glycoproteins that are anchored within the lipid membrane envelope. One or more glycoproteins from a given virus may form a glycoprotein complex that interacts with a number of different surface and/or intracellular receptors of infected host cells to initiate the association between virus and host cell. However, one glycoprotein is generally denoted as the protein primarily driving the fusion of viral and host cell membranes. At least three distinct classes of viral membrane fusion proteins have been determined (classes I, II, and III) [Weissenhorn, W.; Carfi, A.; Lee, K. H.; Skehel, J. J., and Wiley, D. C. Crystal structure of the Ebola virus membrane fusion subunit, GP2, from the envelope glycoprotein ectodomain. Mol. Cell (1998) 2:605-616; White, J. M.; Delos, S. E.; Brecher, M.; Schornberg K. Structures and mechanisms of viral membrane fusion proteins: multiple variations on a common theme. Crit. Rev. Biochem. Mol. Biol. (2008) 43:189-219; Igonet, S.; Vaney, M. C.; Vonrhein, C.; Bricogne, G.; Stura, E. A.; Hengartner H.; Eschli, B.; Rey, F. A. X-ray structure of the arenavirus glycoprotein GP2 in its postfusion hairpin conformation, Proc. Natl. Acad. Sci. (2011) 108:19967-19972]. The above papers are herein incorporated by reference in their entirety for all purposes. Class I fusion proteins are found in viruses from the Orthomyxoviridae, Retroviridae, Paramyxoviridae, Coronaviridae, Filoviridae, and Arenaviridae familes, Class II proteins from Togaviridae, Flaviviridae, and Bunyaviridae while Class III or other types are from Rhadboviridae, Herpesviridae, Poxviridae, and Hepadnaviridae.

Given that viral cell entry is an essential step in the viral replication process the identification of compounds that inhibit virus cell entry could provide attractive antivirals for viruses that are pathogenic to humans and/or other mammals. Chemical compounds that act as inhibitors of one enveloped virus may also act as inhibitors of other enveloped viruses. However, while enveloped

TABLE 1
Family and Genera of Envelope Viruses
and Glycoprotein Classification

| | | | Glyco- |
|---|---|---|---|
| Envelope Virus | | Examples of | protein |
| Family | Genera | pathogenic species | Class |
| Orthomyxoviridae | Influenza virus | Influenza A virus | I |
| | A, B, C | | |
| Filoviridae | Ebolavirus | Zaire virus; | I |
| | | Bundibugyo; Sudan; | |
| | | Tai Forest | |
| | Marburgvirus | Marburg virus | |
| Arenaviridae | Mammarenavirus | Lassa virus; Junin; | I |
| | | Machupo; Guanarito | |
| Coronaviridae | Betacoronaviruses | SARS virus; MERS; | I |
| | | HKU-1; OC43 | |
| Flaviviridae | Flavivirus | Dengue virus; Yellow | II |
| | | Fever; West Nile; | |
| | | Japanese encephalitis | |
| Bunyaviridae | Hantavirus | Andes virus | II |
| | Orthobunyavirus | Bunyamwera virus | |
| | Phlebovirus | Rift Valley fever virus | |
| | Nairovirus | Crimean-Congo | |
| | | hemorrhagic fever | |
| | | virus | |
| Togaviridae | Alphavirus | Chikungunya virus; | II |
| | | Sindbis virus | |
| Paramyxoviridae | Rubulavirus | Mumps virus | I |
| | Morbillivirus | Measles virus | |
| | Pneumovirus | Respiratory syncitial | |
| | | virus | |
| | Henipavirus | Hendra virus; Nipah | |
| Herpesviridae | Cytomegalovirus | Human CMV | III |
| | Simplexvirus | HSV-1; HSV-2 | |
| | Varicellovirus | HHV-3 (Varicella | |
| | | zoster virus) | |
| | Roseolovirus | HHV-6; HHV-7 | |
| | Lymphocryptovirus | Epstein-Barr virus | |
| | Rhadinovirus | Kaposi's sarcoma- | |
| | | associated herpesvirus | |

viruses share some common functional and structural features with regard to glycoprotein-dependent cell entry and fusion the specific host targets and mechanisms of cell entry differ among enveloped viruses: between and even within different virus families as a function of their unique glycoprotein (GP) sequences and structures, and the cellular host proteins that they interact with [White, J. M.; Delos, S. E.; Brecher, M., Schornberg K. Structures and mechanisms of viralmembrane fusion proteins: multiple variations on a common theme. Crit. Rev. Biochem. Mol. Biol. (2008) 43:189-219]. The above paper is herein incorporated by reference in its entirety for all purposes. The invention described herein relates to the use of compounds for the treatment and/or prophylaxis of infection as mediated by the cell entry and fusion process of filovirus glycoproteins whether native or engineered.

One viral expression system that may be utilized to identify inhibitors of enveloped viruses based on their glycoprotein sequences and functional properties is the vesicular stomatitis virus (VSV) system. This approach uses VSV, a virus in the Rhadboviridae family (expressing Class III fusion proteins), lacking a native VSV glycoprotein. “Pseudotyped” viruses that are infective and functionally replicative in cell culture can be generated by substituting the VSV glycoprotein with a glycoprotein originating from other enveloped viruses. The cell entry properties and functions of these pseudotyped viruses are determined by the viral glycoprotein that has been introduced. The cell entry and infectivity properites of pseudotyped VSV viruses have been shown to be determined by the introduced glycoprotein from a host of envelope viruses including Ebola, Lassa, Hanta, Hepatitis B, and other viruses [Ogino, M., et al. Use of vesicular stomatitis virus pseudotypes bearing hantaan or seoul virus envelope proteins in a rapid and safe neutralization test. Clin. Diagn. Lab. Immunol. (2003) 10(1):154-60; Saha, M. N., et al., Formation of vesicular stomatitis virus pseudotypes bearing surface proteins of hepatitis B virus. J. Virol. (2005) 79(19):12566-74; Takada, A., et al., A system for functional analysis of Ebola virus glycoprotein, Proc. Natl. Acad. Sci. (1997) 94:14764-69; Garbutt, M., et al., Properties of replication-competent vesicular stomatitis virus vectors expressing glycoproteins of filoviruses and arenaviruses. J. Virol. (2004) 78(10):5458-65]. The above papers are herein incorporated by reference in their entirety for all purposes. When the pseudotype virion also expresses a reporter gene such as green fluorescent protein (GFP) or Renilla luciferase, virion infectivity and replication may be monitored using high-throughput optical methods in cultured mammalian cell lines, including Vero and HEK-293 cells [Cote, M.; Misasi, J.; Ren, T.; Bruchez, A., Lee, K., Filone, C. M.; Hensley, L.; Li, Q.; Ory, D.; Chandran, K.; Cunningham, J., Small molecule inhibitors reveal Niemann-Pick C1 is essential for Ebola virus infection, Nature (2011) 477: 344-348]. The above paper is herein incorporated by reference in its entirety for all purposes. While VSV does not infect humans and may not be a virus of particular interest for the development of therapeutic antivirals, VSV pseudotyped viruses expressing glycoproteins from other enveloped viruses may be used to screen chemical libraries to identify compounds that inhibit the glycoproteins, cell entry, and infectivity of enveloped viruses associated with significant human health concerns. [Cunningham, J. et al. US patent application, publication number US2013/0231332; WO 2012/031090, 8 Mar. 2012; WO2013/022550, 14 Feb. 2013; Warren, T. K., et al. Antiviral activity of a small-molecule inhibitor of Filovirus infection. Antimicrob. Agents Chemother. (2010) 54: 2152-2159; Yermolina, M., et al. Discovery, synthesis, and biological evaluation of a novel group of selective inhibitors of filovirus entry. J. Med. Chem. (2011) 54: 765-781; Basu, A., et al. Identification of a small-molecule entry inhibitor for Filoviruses. J. Virol. (2011) 85: 3106-3119; Lee, K., et al., Inhibition of Ebola virus infection: identification of Niemann-Pick as the target by optimization of a chemical probe. ACS Med. Chem. Lett. (2013) 4: 239-243; Madrid, P. B., et al. A Systematic screen of FDA-approved drugs for inhibitors of biological threat agents Plos One (2013) 8: 1-14; Elshabrawy, H. A., et al. Identification of a broad-spectrum antiviral small molecule against severe scute respiratory syndrome Coronavirus and Ebola, Hendra, and Nipah Viruses by using a novel high-throughput screening assay. J. Virol. (2014) 88: 4353-4365]. The above papers and patent application are herein incorporated by reference in their entirety for all purposes.

Filovirus infections are associated with hemorrhagic fevers, the clinical manifestations of which may be severe and/or fatal. As described herein, for the current invention, VSV pseudotyped viruses expressing filovirus glycoproteins can be generated and screened with a collection of chemical compounds to identify those compounds that inhibit infectivity. The identification of inhibitors of filovirus glycoprotein-mediated virus cell entry may be utilized to treat infections of filoviruses to provide effective therapeutic regimens for the prophylaxis and/or treatment of filoviruses or any newly emerging virus, whether native or engineered, whose cell entry properties may be determined by filovirus glycoproteins.

The Filoviridae virus family is comprised of at least three genera: Ebolavirus, which currently includes five species Zaire (EBOV), Sudan (SUDV), Bundibygo (BDBV), Tai Forest (TAFV) and Reston (RESTV), Marburgvirus, which currently includes two species Marburg (MARV) and Ravn (RAVV), and Cuervavirus, which currently includes a single species LLovia virus (LLOV). RAVV and LLOV are examples of filoviruses that have been identified only recently and a number of additional new species and genera may continue to emerge.

TABLE 2
Family Filoviridae: currently identified fiiovirus
genera, species, and nomenclature

| Genus name | Species name | Virus name (Abbreviation) |
|---|---|---|
| Cuevavirus | Lloviu cuevavirus | Lloviu virus (LLOV) |
| Ebolavirus | Bundibugyo ebolavirus | Bundibugyo virus (BDBV) |
| | Reston ebolavirus | Reston virus (RESTV) |
| | Sudan ebolavirus | Sudan virus (SUDV) |
| | Taï Forest ebolavirus | Taï Forest virus (TAFV) |
| | Zaire ebolavirus | Ebola virus (EBOV) |
| Marburgvirus | Marburg marburgvirus | Marburg virus (MARV) |
| | | Raven virus (RAVV) |

Glycoproteins from Filoviridae family members can be expressed in pseudotyped viruses (e.g. VSV pseudotype) to identify compounds that inhibit filovirus infection. Based on the structural similarities and/or differences between the viral glycoprotein target and/or host cell targets, the inhibitor compounds may act on only a single filovirus glycoprotein or on a broad spectrum of filoviruses. Furthermore, given the basic functional and structural similarities of glycoproteins among different families of enveloped viruses it is proable that a given compound class may act across a broad range of enveloped viruses.

Alignments of representative filovirus glycoprotein sequences were generated to illustrate the amino acid homology among different filovirus species.

TABLE 3
Homology of filovirus glycoproteins - created by Clustal2.1

| Species/Genbank ID | Zaire | Bundi | T Forest | Reston | Sudan | Marburg |
|---|---|---|---|---|---|---|
| Zaire/AAB81004 | 100 | 68.0 | 66.5 | 59.9 | 56.8 | 32.7 |
| Bundibugyo/AGL73453 | 68.0 | 100 | 73.6 | 60.4 | 57.7 | 33.0 |
| Tai Forest/YP_003815426 | 66.5 | 73.6 | 100 | 59.5 | 57.7 | 33.9 |
| Reston/BAB69006 | 59.9 | 60.4 | 59.5 | 100 | 61.4 | 32.7 |
| Sudan/AAB37096 | 56.8 | 57.7 | 57.7 | 61.4 | 100 | 33.0 |
| Marburg/AAC40460 | 32.7 | 33.0 | 33.9 | 32.7 | 33.0 | 100 |

A matrix comparison of the amino acid homology (homology is defined as the number of identities between any two sequences, divided by the length of the alignment, and represented as a percentage) as determined from the Clustal2.1 program (http://www.ebi.ac.uk/Tools/msa/clustalo/) among and between distinct filovirus genus and species is illustrated in Table 3. Glycoproteins among virus species within the same filovirus genus (e.g., Ebolavirus) are more homologous to each other than to those in another genus (Marburgvirus). However, currently available filovirus glycoproteins exhibit significant homology (>30% identity from any one member to another). Given this homology for some chemical series it is possible to identify compounds that exhibit activity against a broad-spectrum of filoviruses.

Similar alignments were subsequently carried out with a number of class I glycoproteins from other enveloped virus families. Each of the glycoproteins from the other enveloped viruses exhibit <20% identity with any of the filovirus glycoproteins. Although there are similarities in functional and structural characteristics among the class I glycoproteins, there are clear distinctions including dependence on low pH, receptor binding, location of the fusion peptide [White, J. M.; Delos, S. E.; Brecher, M.; Schornberg, K. Structures and mechanisms of viral membrane fusion proteins: multiple variations on a common theme. Crit. Rev. Biochem. Mol., Biol. (2008) 43:189-219] and given the low amino acid sequence homology across class I virus families it becomes unlikely that a given chemical series that inhibits filovirus cell entry/fusion would also exhibit similar inhibitory activities with other envelope class I glycoprotein virus families. The above paper is herein incorporated by reference in its entirety for all purposes.

TABLE 4
Homology matrix between filoviruses and other class
I glycoprotein viruses-created by Clustal2.1

| | | | | | | | | | | | |
|---|---|---|---|---|---|---|---|---|---|---|---|
| Z AAB81004 | 100 | 66.5 | 68.0 | 56.8 | 59.9 | 32.7 | 17.0 | 12.8 | 13.4 | 14.2 | 13.7 |
| T YP_003815426 | 66.5 | 100 | 73.6 | 57.7 | 59.5 | 33.9 | 17.7 | 12.0 | 12.0 | 13.8 | 14.2 |
| B AGL73453 | 68.0 | 73.6 | 100 | 57.7 | 60.4 | 33.0 | 17.9 | 12.3 | 12.3 | 13.4 | 14.7 |
| S AAB37096 | 56.8 | 57.7 | 57.7 | 100 | 61.4 | 33.0 | 16.4 | 12.9 | 13.0 | 14.8 | 12.8 |
| R BAB69006 | 59.9 | 59.5 | 60.4 | 61.4 | 100 | 32.7 | 19.8 | 12.9 | 11.8 | 14.6 | 13.5 |
| M AAC40460 | 32.7 | 33.9 | 33.0 | 33.0 | 32.7 | 100 | 15.7 | 10.7 | 8.7 | 12.2 | 14.1 |
| INF ACP41105 | 17.0 | 17.7 | 17.9 | 16.4 | 19.8 | 15.7 | 100 | 14.5 | 12.6 | 11.8 | 11.2 |
| LASV NP_694870 | 12.8 | 12.0 | 12.3 | 12.9 | 12.9 | 10.7 | 14.5 | 100 | 43.2 | 18.8 | 18.3 |
| JUNV AY619641 | 13.4 | 12.0 | 12.3 | 13.0 | 11.8 | 8.7 | 12.6 | 43.2 | 100 | 15.2 | 14.3 |
| Nipah AP238467 | 14.2 | 13.8 | 13.4 | 14.8 | 14.6 | 12.2 | 11.8 | 18.8 | 15.2 | 100 | 20.8 |
| Measles AF21882 | 13.7 | 14.2 | 14.7 | 12.8 | 11.2 | 14.1 | 13.5 | 18.3 | 14.3 | 20.8 | 100 |

Abbreviations: M: Marburg, Z: Zaire, T: Tai Forest, B: Bundibugyo, S: Sudan, R: Reston, INF: Influenza, LASV: Lassa virus, JUNV: Junin virus; Genbank ID in bold

It was surprisingly discovered that the compounds of the invention showed broad-spectrum inhibition of viruses expressing a range of Ebolavirus glycoproteins.

### Optical Activity of Adamantane Derivatives.

The four bridgehead positions of adamantane are formally analogous to the four tetrahedral valances of carbon. Adamantanes with four different bridgehead substituents are therefore chiral. [Bingham, R. C.; Schleyer, P. R. (1971) Recent developments in the chemistry of adamantane and related polycyclic hydrocarbons. In: Chemistry of Adamantanes. Fortschritte der Chemischen Forschung, vol. 18/1. Springer, Berlin, Heidelberg]. The above paper is herein incorporated by reference in its entirety for all purposes. Optically active adamantanecarboxylic acids have been prepared through resolution of the racemic acid with amines [Hamill, H.; McKervey, M. A. The resolution of 3-methyl-5-bromoadamantane carboxylic acid. Chem. Comm. 1969, 864; Applequist, J.; Rivers, P., Applequist, D. E. Theoretical and experimental studies of optically active bridgehead-substituted adamantanes and related compounds. J. Am. Chem. Soc. 1969, 91, 5705 5711] and via derivatization of the racemic acid using a chiral auxiliary [Aoyama, M; Hara, S. Synthesis of optically active fluoroadamantane derivatives having different substituents on the tert-carbons and its use as non-racemizable source for new optically active adamantane derivatives. Tetrahedron 2013, 69, 10357-10360]. The above papers are herein incorporated by reference in their entirety for all purposes.

Complexes formed between a protein and two enantiomers are diastereomers, and as a result have different chemical properties. Therefore, dissociation constants between the protein and the the two enantiomers may differ and even involve different binding sites [Silverman, R. B., Holladay, M. W. Drug receptor and chirality. In: The organic chemistry of drug design and drug action, 3rd ed.; Academic Press, Amsterdam, Boston, 2014, p. 140-145, Academic Press. Amsterdam]. The above book is herein incorporated by reference in its entirety for all purposes. According to the nomenclature by Ariens, when there is isomeric stereoselectivity, the more potent isomer is termed the “eutomer”, and the less potent isomer is called the “distomer” [Ariens, E. J. Stereochemistry: a source of problems in medicinal chemistry. Med. Res. Rev. 1986, 6, 451-466. Stereochemistry in the analysis of drug action, part II. Med. Chem Rev. 1987, 7, 367]. The above paper is herein incorporated by reference in its entirety for all purposes. The ratio of the potency of the more potent enantiomer to the less potent enantiomer is termed “eudistic ratio”.

In the present invention, we have prepared single enantiomers of certain racematic mixtures of amides and thioamides of adamantane carboxylic acids and surprisingly discovered that one enantiomer was more potent compared to the opposite enantiomer for Ebolavirus.

### DESCRIPTION OF THE DRAWINGS

None

### BRIEF DESCRIPTION OF THE INVENTION

The present invention relates to methods of inhibiting filoviruses (or any virus whose cell entry is mediated by filovirus glycoproteins) infection in humans, other mammals, or in cell culture, to treating filovirus infection, to methods of inhibiting the replication of filoviruses, to methods of reducing the amount of filoviruses in mammals, and to compositions that can be employed for such methods. These methods, applications, and compositions apply not only to Filoviridae viruses but also to any virus, whether naturally emerging or engineered, whose cell entry properties are determined by filovirus glycoproteins.

In one embodiment, the method comprises administering to humans, other mammals, cell culture, or biological sample an effective amount of a compound represented by Structural Formulae I, II, III, IV, V, VI, VIa, VIb, VII, and VIII for treatment of Ebolavirus infection

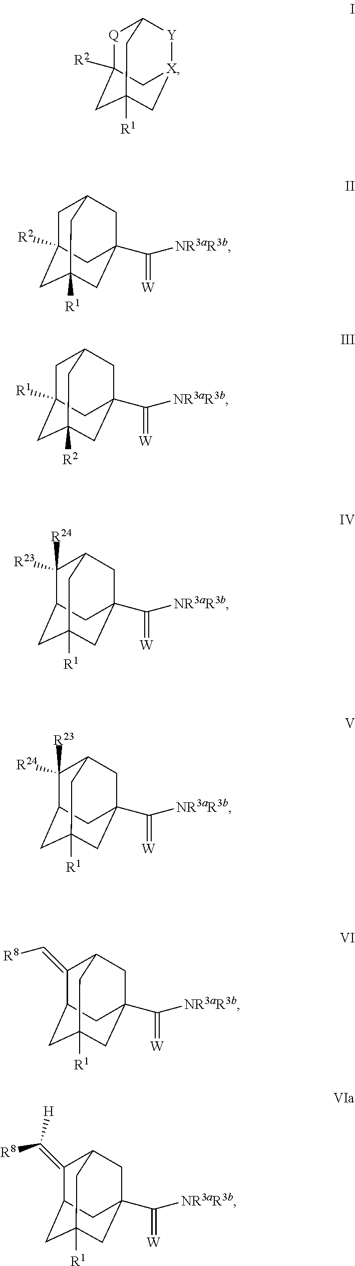

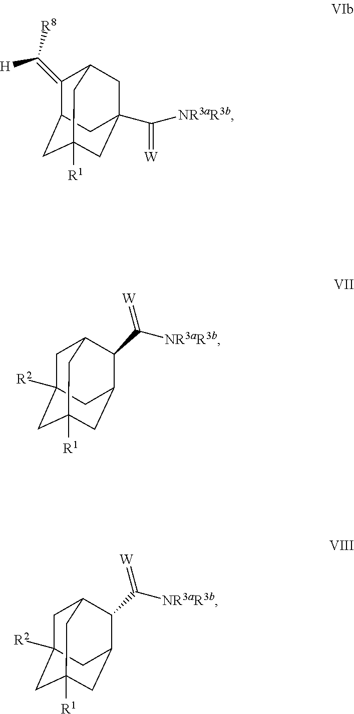

or a pharmaceutically acceptable salt, and a pharmaceutically acceptable carrier, diluent, or vehicle thereof, wherein:
X is

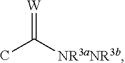

Y is CH₂, and Q is CH₂ or CR²³R²⁴; or
X is CH, Y is

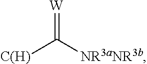

### and Q is CH₂;

W is selected from the group consisting of O and S;
and when X is

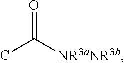

Y is CH₂, and Q is CH₂, then
R¹ is selected from (C₆ to C₁₀) aryl and (C₂ to C₉) heteroaryl, wherein
each of the said (C₆ to C₁₀) aryl and (C₂ to C₉) heteroaryl is optionally substituted with at least one R⁸ group;
R² is selected from the group consisting of

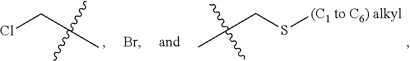

and
NR^{3a}R^{3b} is selected from the group consisting of the substituents of Table 5;

TABLE 5

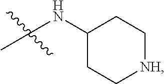 [SMILES: *NC1CCNCC1]

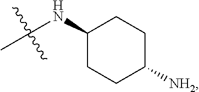 [SMILES: *N[C@H]1CC[C@H](N)CC1]

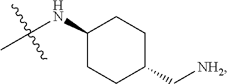 [SMILES: *N[C@H]1CC[C@H](CN)CC1]

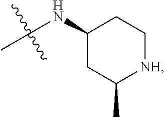 [SMILES: *N[C@H]1CCN[C@@H](C)C1]

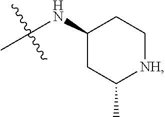 [SMILES: *N[C@H]1CCN[C@H](C)C1]

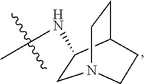 [SMILES: *N[C@H]1CN2CCC1CC2]

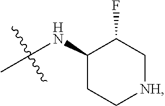 [SMILES: *N[C@@H]1CCNC[C@H]1F]

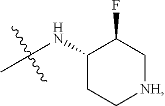 [SMILES: *N[C@H]1CCNC[C@@H]1F]

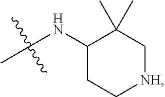 [SMILES: *NC1CCNCC1(C)C]

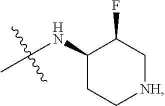 [SMILES: *N[C@@H]1CCNC[C@@H]1F]

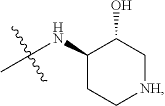 [SMILES: *N[C@@H]1CCNC[C@H]1O]

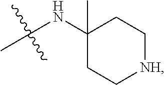 [SMILES: *NC1(C)CCNCC1]

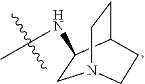 [SMILES: *N[C@@H]1CN2CCC1CC2]

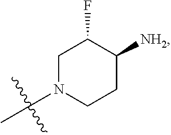 [SMILES: *N1CC[C@H](N)[C@@H](F)C1]

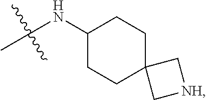 [SMILES: *NC1CCC2(CC1)CNC2]

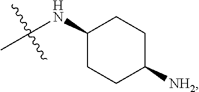 [SMILES: *N[C@H]1CC[C@@H](N)CC1]

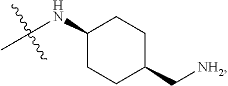 [SMILES: *N[C@H]1CC[C@@H](CN)CC1]

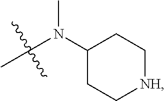 [SMILES: *N(C)C1CCNCC1]

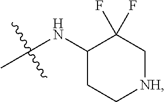 [SMILES: *NC1CCNCC1(F)F]

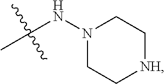 [SMILES: *NN1CCNCC1]

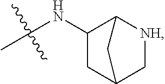 [SMILES: *NC1CC2CNC1C2]

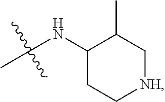 [SMILES: *NC1CCNCC1C]

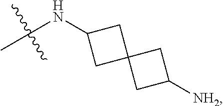 [SMILES: *NC1CC2(CC(N)C2)C1]

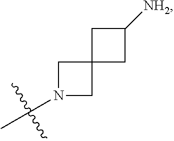 [SMILES: *N1CC2(CC(N)C2)C1]

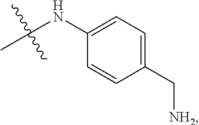 [SMILES: *Nc1ccc(CN)cc1]

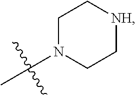 [SMILES: *N1CCNCC1]

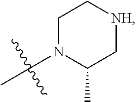 [SMILES: *N1CCNC[C@@H]1C]

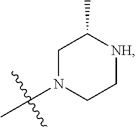 [SMILES: *N1CCN[C@@H](C)C1]

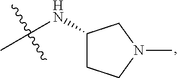 [SMILES: *N[C@H]1CCN(C)C1]

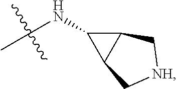 [SMILES: *N[C@H]1[C@@H]2CNC[C@@H]21]

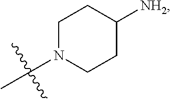 [SMILES: *N1CCC(N)CC1]

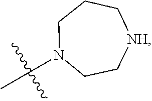 [SMILES: *N1CCCNCC1]

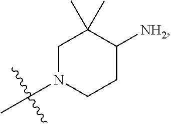 [SMILES: *N1CCC(N)C(C)(C)C1]

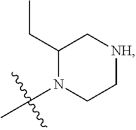 [SMILES: *N1CCNCC1CC]

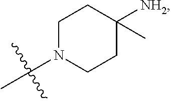 [SMILES: *N1CCC(C)(N)CC1]

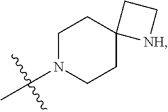 [SMILES: *N1CCC2(CCN2)CC1]

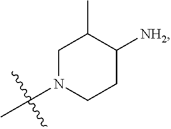 [SMILES: *N1CCC(N)C(C)C1]

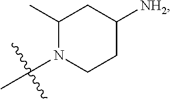 [SMILES: *N1CCC(N)CC1C]

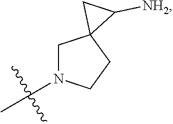 [SMILES: *N1CCC2(CC2N)C1]

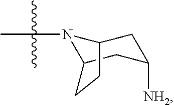 [SMILES: *N1C2CCC1CC(N)C2]

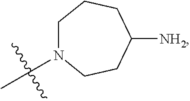 [SMILES: *N1CCCC(N)CC1]

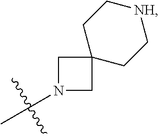 [SMILES: *N1CC2(CCNCC2)C1]

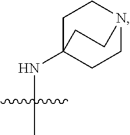 [SMILES: *NC12CCN(CC1)CC2]

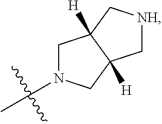 [SMILES: *N1C[C@H]2CNC[C@H]2C1]

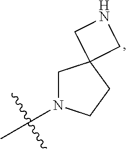 [SMILES: *N1CCC2(CNC2)C1]

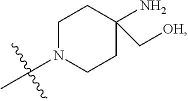 [SMILES: *N1CCC(N)(CO)CC1]

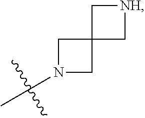 [SMILES: *N1CC2(CNC2)C1]

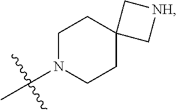 [SMILES: *N1CCC2(CC1)CNC2]

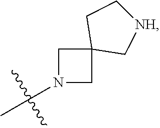 [SMILES: *N1CC2(CCNC2)C1]

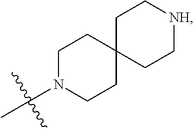 [SMILES: *N1CCC2(CCNCC2)CC1]

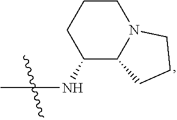 [SMILES: *N[C@@H]1CCCN2CCC[C@H]12]

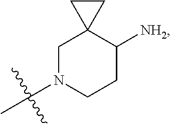 [SMILES: *N1CCC(N)C2(CC2)C1]

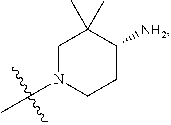 [SMILES: *N1CC[C@@H](N)C(C)(C)C1]

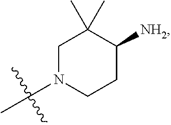 [SMILES: *N1CC[C@H](N)C(C)(C)C1]

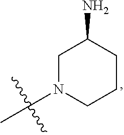 [SMILES: *N1CCC[C@H](N)C1]

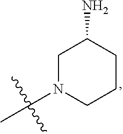 [SMILES: *N1CCC[C@@H](N)C1]

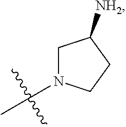 [SMILES: *N1CC[C@H](N)C1]

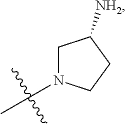 [SMILES: *N1CC[C@@H](N)C1]

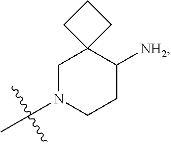 [SMILES: *N1CCC(N)C2(CCC2)C1]

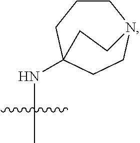 [SMILES: *NC12CCCN(CC1)CC2]

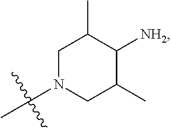 [SMILES: *N1CC(C)C(N)C(C)C1]

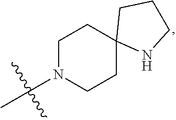 [SMILES: *N1CCC2(CCCN2)CC1]

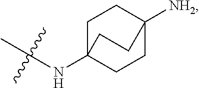 [SMILES: *NC12CCC(N)(CC1)CC2]

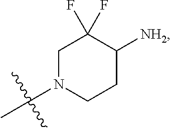 [SMILES: *N1CCC(N)C(F)(F)C1]

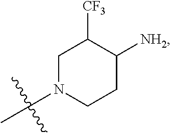 [SMILES: *N1CCC(N)C(C(F)(F)F)C1]

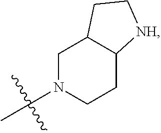 [SMILES: *N1CCC2NCCC2C1]

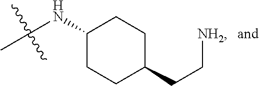 [SMILES: *N[C@H]1CC[C@H](CCN)CC1 ; "and"]

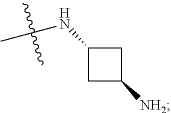 [SMILES: *N[C@H]1C[C@H](N)C1]

and when X is CH, Y is

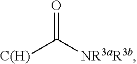

and Q is CH₂, then
R¹ is selected from (C₆ to C₁₀) aryl and (C₂ to C₉) heteroaryl, wherein
each of the said (C₆ to C₁₀) aryl and (C₂ to C₉) heteroaryl is optionally substituted with at least one R⁸ group;
R² is selected from the group consisting of halogen, methyl, ethyl, propyl, chloromethyl, chloroethyl, methylthiomethyl, and thiomethyl;
NR^{3a}R^{3b} is selected from the group consisting of the substituents of Table 5;
and when X is

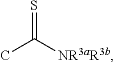

Y is CH₂, and Q is CH₂; or
X is CH, Y is

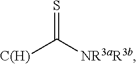

### and Q is CH₂; or

X is

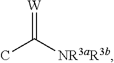

Y is CH₂, W is O or S, and Q is CR²³R²⁴ then
R¹ is selected from (C₆ to C₁₀) aryl and (C₂ to C₉) heteroaryl, wherein
each of the said (C₆ to C₁₀) aryl and (C₂ to C₉) heteroaryl is optionally substituted with at least one R⁸ group;
and when Q is CH₂, then
R² is selected from hydrogen, halogen, OH, nitro, CF₃, —NR^{6a}R^{6b}, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, —C(O)R⁷, —C(O)NR^{6a}R^{6b}, —S(O)ₘR⁷, —S(O)ₘNR^{6a}R^{6b}, —NR^{6a}S(O)ₘR⁷, —(CH₂)ₙC(O)OR⁷, —(CH₂)ₙC(O)N(R^{6a}R^{6b}), —(CH₂)ₙN(R^{6a}R^{6b}), —OC(O)R⁷, —NR^{6a}C(O)R⁷, and —NR^{6a}C(O)N(R^{6a}R^{6b}), wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁸ group;
and when Q is CR²³R²⁴ then R² is hydrogen;
NR^{3a}R^{3b} is selected from the group consisting of

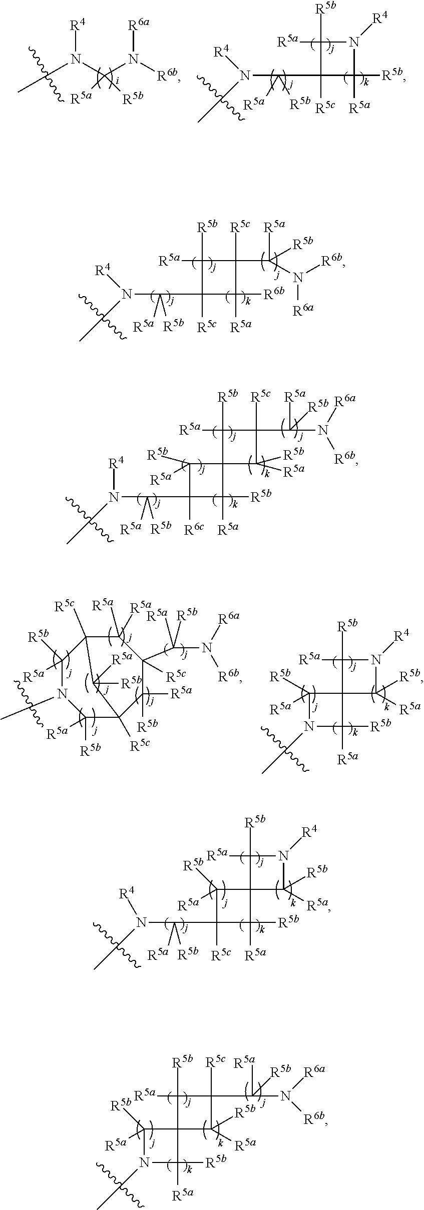

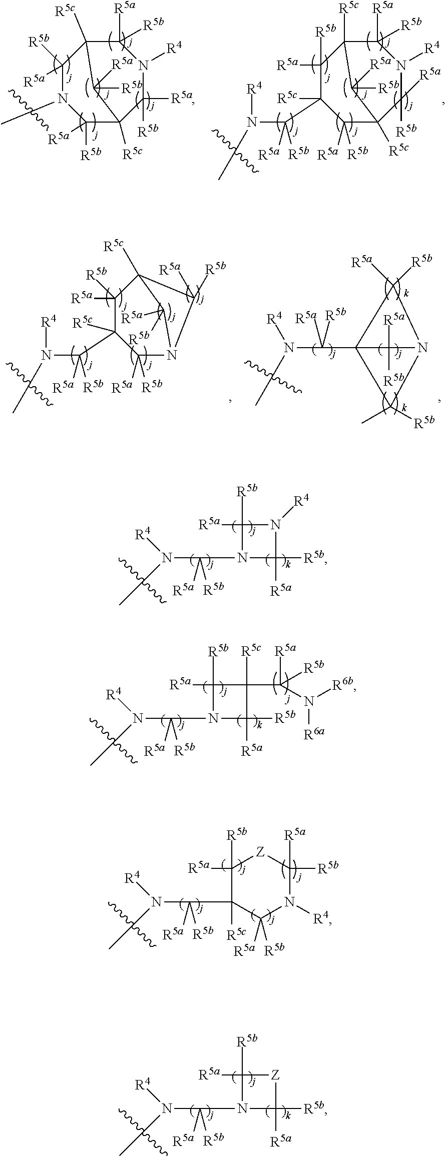

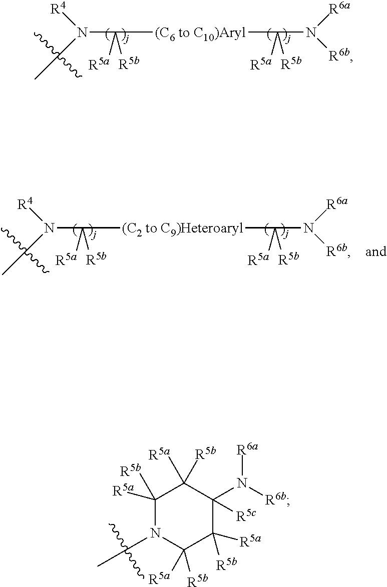

Z is selected from the group consisting of —O—, —S—, —S(O)—, and —S(O)₂—;
each R⁴ is independently selected from hydrogen, (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, (C₆ to C₁₀) arylene, and (C₂ to C₉) heteroarylene, wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, (C₆ to C₁₀) arylene, and (C₂ to C₉) heteroarylene is optionally substituted with at least one R⁸ group;
each of the R^{5a}, R^{5b}, and R^{5c} is independently selected from hydrogen, halogen, OH, nitro, CF₃, —NR^{6a}R^{6b}, (C₁ to C₁₀) alkyl, (C₂ to C₁₀) alkenyl, (C₂ to C₁₀) alkynyl, (C₆ to C₁₀) alkoxy, aryloxy, cyano, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, —C(O)R⁷, —C(O)NR^{6a}R^{6b}, —S(O)ₘR⁷, —S(O)ₘNR^{6a}R^{6b}, —NR^{6a}S(O)ₘR⁷, —(CH₂)ₙC(O)OR⁷, —(CH₂)ₙC(O)N(R^{6a}R^{6b}), —(CH₂)ₙN(R^{6a}R^{6b}), —OC(O)R⁷, —NR^{6a}C(O)R⁷, and —NR^{6a}C(O)N(R^{6a}R^{6b}), wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁸ group;
or R^{5a} and R^{5b} may be taken together with the carbon atom to which they are attached to form a (C₃ to C₁₀) cycloalkyl ring, wherein
the said (C₃ to C₁₀) cycloalkyl ring is optionally substituted with at least one R⁸ group;
each of the R^{6a} and R^{6b} is independently selected from hydrogen, (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, (C₆ to C₁₀) arylene, and (C₂ to C₉) heteroarylene, wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, (C₆ to C₁₀) arylene, and (C₂ to C₉) heteroarylene is optionally substituted with at least one R⁸ group,
or R^{6a} and R^{6b} may be taken together with the nitrogen atom to which they are attached to form a (C₂ to C₁₀) cycloheteroalkyl ring, wherein
said (C₂ to C₁₀) cycloheteroalkyl ring has 1 to 3 ring heteroatoms selected from the group consisting of N, O, and S, and wherein
the said (C₂ to C₁₀) cycloheteroalkyl ring is optionally substituted with at least one R⁸ group;
each of the R⁷ is independently selected from hydrogen, (C₁ to C₁₀) alkyl, (C₂ to C₁₀) alkenyl, (C₂ to C₁₀) alkynyl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl, wherein
   each of the said (C₁ to C₁₀) alkyl, (C₂ to C₁₀) alkenyl, (C₂ to C₁₀) alkynyl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁸ group;
each R⁸ is independently selected from hydrogen, halogen, OH, nitro, CF₃, —NR^{9a}R^{9b}, oxo, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, (C₆ to C₁₀) arylene, (C₂ to C₉) heteroarylene, (C₃ to C₁₀) cycloalkylene, (C₂ to C₁₀) cycloheteroalkylene, —C(O)R¹⁰, —C(O)NR^{9a}R^{9b}, —S(O)ₘR¹⁰, —S(O)ₘNR^{9a}R^{9b}, —NR^{9a}S(O)ₘR¹⁰, —(CH₂)ₙC(O)OR¹⁰, —(CH₂)ₙC(O)N(R^{9a}R^{9b}), —(CH₂)ₙN(R^{9a}R^{9b}), —OC(O)R¹⁵, —O(CH₂)ₙO—, —NR^{9a}C(O)R^{1′}, and —NR^{9a}C(O)N(R^{9a}R^{9b}), wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, (C₂ to C₉) heteroarylene, (C₃ to C₁₀) cycloalkylene, and (C₂ to C₁₀) cycloheteroalkylene is optionally substituted with at least one R¹¹ group;
each of the R^{9a} and R^{9b} is independently selected from hydrogen, (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, (C₆ to C₁₀) arylene, and (C₂ to C₉) heteroarylene, wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, and (C₂ to C₉) heteroarylene is optionally substituted with at least one R¹¹ group,
or R^{9a} and R^{9b} may be taken together with the nitrogen atom to which they are attached to form a (C₂ to C₁₀) cycloheteroalkyl ring, wherein
said (C₂ to C₁₀) cycloheteroalkyl ring has 1 to 3 ring heteroatoms selected from the group consisting of N, O, and S, and wherein
the said (C₂ to C₁₀) cycloheteroalkyl ring is optionally substituted with at least one R¹ group;
each R¹⁰ is independently selected from hydrogen, (C₁ to C₁₀) alkyl, (C₂ to C₁₀) alkenyl, (C₂ to C₁₀) alkynyl, (C₃ to C₁₀) cycloalkyl, (C₅ to C₁₀) cycloalkenyl, (C₂ to C₁₀) cycloheteroalkyl, (C₆ to C₁₀) aryl, and (C₂ to C₉) heteroaryl, wherein
each of the said (C₁ to C₁₀) alkyl, (C₂ to C₁₀) alkenyl, (C₂ to C₁₀) alkynyl, (C₃ to C₁₀) cycloalkyl, (C₅ to C₁₀) cycloalkenyl, (C₂ to C₁₀) cycloheteroalkyl, (C₆ to C₁₀) aryl, and (C₂ to C₉) heteroaryl is optionally substituted with at least one R¹¹ group;
each R¹¹ is independently selected from hydrogen, halogen, OH, nitro, CF₃, —NR^{12a}R^{12b}, oxo, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, (C₂ to C₉) heteroarylene, (C₃ to C₁₀) cycloalkylene, (C₂ to C₁₀) cycloheteroalkylene, —C(O)R¹⁸, —C(O)NR^{12a}R^{12b}, —S(O)ₘR¹³, —S(O)ₘNR^{12a}R^{12b}, —NR^{12a}S(O)ₘR¹³, —(CH₂)ₙC(O)OR¹³, —(CH₂)ₙC(O)N(R^{12a}R^{12b}), —(CH₂)ₙN(R^{12a}R^{12b}), —OC(O)R¹³, —NR^{12a}C(O)R¹³, and —NR^{12a}C(O)N(R^{12a}R^{12b}), wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₂ to C₉) heteroaryl, (C₆ to C₁₀) aryl, (C₆ to C₁₀) arylene, (C₂ to C₉) heteroarylene, (C₃ to C₁₀) cycloalkylene, and (C₂ to C₁₀) cycloheteroalkylene is optionally substituted with at least one R¹⁴ group;
each of the R^{12a} and R^{12b} is independently selected from hydrogen, (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, and (C₂ to C₉) heteroarylene, wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, and (C₂ to C₉) heteroarylene is optionally substituted with at least one R¹⁴ group,
or R^{12a} and R^{12b} may be taken together with the nitrogen atom to which they are attached to form a (C₂ to C₁₀) cycloheteroalkyl ring, wherein
said (C₂ to C₁₀) cycloheteroalkyl ring has 1 to 3 ring heteroatoms selected from the group consisting of N, O, and S, and wherein
the said (C₂ to C₁₀) cycloheteroalkyl ring is optionally substituted with at least one R¹⁴ group;
each R¹³ is independently selected from hydrogen, halogen, OH, nitro, CF₃, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₁₀) cycloheteroalkyl, (C₆ to C₁₀) aryl, and (C₂ to C₉) heteroaryl, wherein
each of the said (C₁ to C₁₀) alkyl, (C₂ to C₁₀) alkenyl, (C₂ to C₁₀) alkynyl, (C₃ to C₁₀) cycloalkyl, (C₅ to C₁₀) cycloalkenyl, (C₂ to C₁₀) cycloheteroalkyl, (C₂ to C₉) heteroaryl, and (C₆ to C₁₀) aryl is optionally substituted with at least one R¹⁴ group;
each R¹⁴ is independently selected from hydrogen, halogen, OH, nitro, CF₃, —NR^{15a}R^{15b}, oxo, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, (C₂ to C₉) heteroarylene, (C₃ to C₁₀) cycloalkylene, (C₂ to C₁₀) cycloheteroalkylene, —C(O)R¹⁶, —C(O)NR^{15a}R^{15b}, —S(O)ₘR¹⁶, —S(O)ₘNR^{15a}R^{15b}, —NR^{15a}S(O)ₘR¹⁶, —(CH₂)ₙC(O)OR¹⁶, —(CH₂)ₙC(O)N(R^{15a}R^{15b}), —(CH₂)ₙN(R^{15a}R^{15b}), —OC(O)R¹⁶, —NR^{15a}C(O)R¹⁶, and —NR^{15a}C(O)N(R^{15a}R^{15b}), wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, (C₂ to C₉) heteroarylene, (C₃ to C₁₀) cycloalkylene, and (C₂ to C₁₀) cycloheteroalkylene is optionally substituted with at least one R¹⁷ group;
each of the R^{15a} and R^{15b} is independently selected from hydrogen, (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, and (C₂ to C₉) heteroaryl, wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, and (C₂ to C₉) heteroaryl is optionally substituted with at least one R¹⁷ group,
or R^{15a} and R^{15b} may be taken together with the nitrogen atom to which they are attached to form a (C₂ to C₁₀) cycloheteroalkyl ring, wherein
said (C₂ to C₁₀) cycloheteroalkyl ring has 1 to 3 ring heteroatoms selected from the group consisting of N, O, and S, and wherein
the said (C₂ to C₁₀) cycloheteroalkyl ring is optionally substituted with at least one R¹⁷ group;
each R¹⁶ is independently selected from hydrogen, halogen, OH, nitro, CF₃, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₁₀) cycloheteroalkyl, (C₆ to C₁₀) aryl, and (C₂ to C₉) heteroaryl;
each R¹⁷ is independently selected from hydrogen, halogen, OH, nitro, CF₃, —NR^{18a}R^{18b}, oxo, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, (C₂ to C₉) heteroarylene, (C₃ to C₁₀) cycloalkylene, (C₂ to C₁₀) cycloheteroalkylene, —C(O)R¹⁹, —C(O)NR^{13a}R^{18b}, —S(O)ₘR¹⁹, —S(O)ₘNR^{18a}R^{18b}, —NR^{18a}S(O)ₘR¹⁹, —(CH₂)ₙC(O)OR¹⁹, —(CH₂)ₙC(O)N(R^{18a}R^{18b}), —(CH₂)ₙN(R^{18a}R^{18b}), —OC(O)R¹⁹, —NR^{18a}C(O)R¹⁹, and —NR^{18a}C(O)N(R^{18a}R^{18b}), wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, (C₂ to C₉) heteroarylene, (C₃ to C₁₀) cycloalkylene, and (C₂ to C₁₀) cycloheteroalkylene is optionally substituted with at least one R²⁰ group;
each of the R^{18a} and R^{18b} is independently selected from hydrogen, (C₁ to C₁₀) alkyl, (C₂ to C₁₀) alkenyl, (C₂ to C₁₀) alkynyl, (C₃ to C₁₀) cycloalkyl, (C₅ to C₁₀) cycloalkenyl, (C₂ to C₁₀) cycloheteroalkyl, (C₂ to C₉) heteroaryl, and (C₆ to C₁₀) aryl;
each R¹⁹ is independently selected from hydrogen, halogen, OH, nitro, CF₃, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl;
each R²⁰ is independently selected from hydrogen, halogen, OH, nitro, CF₃, —NR^{21a}R^{21b}, oxo, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, (C₂ to C₉) heteroarylene, (C₃ to C₁₀) cycloalkylene, (C₂ to C₁₀) cycloheteroalkylene, —C(O)R²², —C(O)NR^{21a}R^{21b}, —S(O)ₘR²², —S(O)ₘNR^{21a}R^{21b}, —NR^{21a}S(O)ₘR²², —(CH₂)ₙC(O)OR²², —(CH₂)ₙC(O)N(R^{21a}R^{21b}), —(CH₂)ₙN(R^{21a}R^{21b}), —OC(O)R²², —NR^{21a}C(O)R²², and —NR^{21a}C(O)N(R^{21a}R^{21b}) wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, (C₂ to C₉) heteroarylene, (C₃ to C₁₀) cycloalkylene, and (C₂ to C₁₀) cycloheteroalkylene is optionally substituted with at least one R²² group;
each of the R^{21a} and R^{21b} is independently selected from hydrogen, (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, and (C₂ to C₉) heteroaryl,
or R^{21a} and R^{21b} may be taken together with the nitrogen atom to which they are attached to form a (C₂ to C₁₀) cycloheteroalkyl ring, wherein
said (C₂ to C₁₀) cycloheteroalkyl ring has 1 to 3 ring heteroatoms selected from the group consisting of N, O, and S;
each R²² is independently selected from hydrogen, halogen, OH, nitro, CF₃, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl;
each R²³ is independently selected from hydrogen, halogen, OH, nitro, CF₃, —NR^{11a}R^{11b}, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cyclo-alkenyl, (C₂ to C₉) cycloheteroalkyl, (C₆ to C₁₀) aryl, (C₂ to C₉) heteroaryl, —C(O)R¹², —C(O)NR^{11a}R^{11b}, —S(O)ₘR¹², —S(O)ₘNR^{11a}R^{11b}, —NR^{11a}S(O)ₘR¹², —(CH₂)ₙC(O)OR¹², —(CH₂)ₙC(O)N(R^{11a}R^{11b}), —(CH₂)ₙN(R^{11a}R^{11b}), —OC(O)R¹², —NR^{11a}C(O)R¹², and —NR^{11a}C(O)N(R^{11a}R^{11b}) wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁸ group;
each R²⁴ is independently selected from halogen, OH, nitro, CF₃, —NR^{11a}R^{11b}, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cyclo-alkenyl, (C₂ to C₉) cycloheteroalkyl, (C₆ to C₁₀) aryl, (C₂ to C₉) heteroaryl, —C(O)R¹², —C(O)NR^{11a}R^{11b}, —S(O)ₘR¹², —S(O)ₘNR^{11a}R^{11b}, —NR^{11a}S(O)ₘR¹², —(CH₂)ₙC(O)OR¹², —(CH₂)ₙC(O)N(R^{11a}R^{11b}), —(CH₂)ₙN(R^{11a}R^{11b}), —OC(O)R¹², —NR^{11a}C(O)R¹², and —NR^{11a}C(O)N(R^{11a}R^{11b}) wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁸ group;
or R²³ and R²⁴ may be taken together with the carbon atom to which they are attached to form a (C₃ to C₁₀) cycloalkyl or (C₂ to C₉) cycloheteroalkyl ring, wherein
the said (C₃ to C₁₀) cycloalkyl or (C₂ to C₉) cycloheteroalkyl ring is optionally substituted with at least one R⁸ group;
or R²³ and R²⁴ may be taken together with the carbon atom to which they are attached to form a carbonyl or alkene, which is optionally substituted with at least one R⁸ group;
i is 2, 3, 4, 5, or 6;
j is 0, 1, 2, 3, 4, or 5;
k is 1, 2, 3, 4, or 5;
m is 0, 1 or 2;
n is 0, 1, 2, 3, or 4;
with the proviso that when R¹ is phenyl, R² is hydrogen, X is

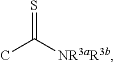

Y is CH₂, and Q is CH₂, then NR^{3a}R^{3b} is not

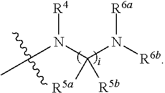

### DESCRIPTION OF THE DRAWINGS

None

### DETAILED DESCRIPTION OF THE INVENTION

The present invention relates to methods of inhibiting filoviruses (or any virus whose cell entry is mediated by filovirus glycoproteins) infection in humans, other mammals, or in cell culture, to treating filovirus infection, to methods of inhibiting the replication of filoviruses, to methods of reducing the amount of filoviruses in mammals, and to compositions that can be employed for such methods. These methods, applications, and compositions apply not only to Filoviridae viruses but also to any virus, whether naturally emerging or engineered, whose cell entry properties are determined by filovirus glycoproteins. The invention also comprises the compounds used in the treatment of filovirus infections.

In one embodiment, the method comprises administering to humans, other mammals, cell culture, or biological sample an effective amount of a compound represented by Structural Formulae I, II, III, IV, V, VI, VIa, VIb, VII, and VIII for treatment of Ebolavirus infection

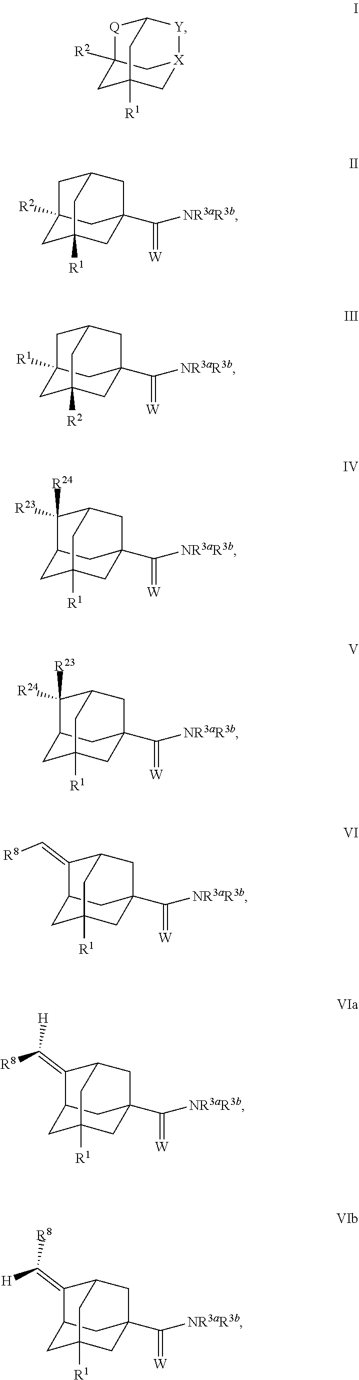

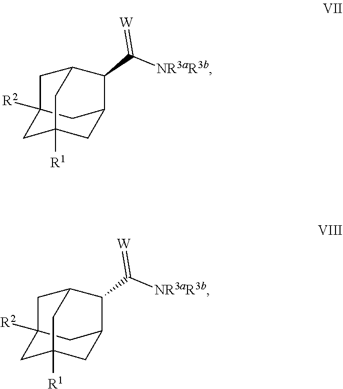

or a pharmaceutically acceptable salt, and a pharmaceutically acceptable carrier, diluent, or vehicle thereof, wherein:
X is

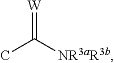

Y is CH₂, and Q is CH₂ or CR²³R²⁴; or
X is CH, Y is

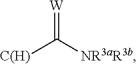

### and Q is CH₂;

W is selected from the group consisting of O and S;
and when X is

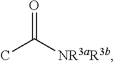

Y is CH₂, and Q is CH₂, then
R¹ is selected from (C₆ to C₁₀) aryl and (C₂ to C₉) heteroaryl, wherein
each of the said (C₆ to C₁₀) aryl and (C₂ to C₉) heteroaryl is optionally substituted with at least one R⁸ group;
R² is selected from the group consisting of

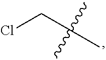 [SMILES: *CCl]

### Br, and

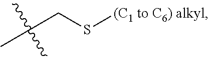

and
NR^{3a}R^{3b} is selected from the group consisting of the substituents of Table 5;
and when X is CH, Y is

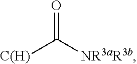

and Q is CH₂, then
R¹ is selected from (C₆ to C₁₀) aryl and (C₂ to C₉) heteroaryl, wherein
each of the said (C₆ to C₁₀) aryl and (C₂ to C₉) heteroaryl is optionally substituted with at least one R⁸ group;
R² is selected from the group consisting of halogen, methyl, ethyl, propyl, chloromethyl, chloroethyl, methylthiomethyl, and thiomethyl;
NR^{3a}R^{3b} is selected from the group consisting of the substituents of Table 5;
and when X is

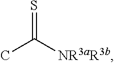

Y is CH₂, and Q is CH₂; or
X is CH, Y is

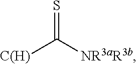

### and Q is CH₂; or

X is

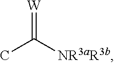

Y is CH₂, W is O or S, and Q is CR²³R²⁴ then
R¹ is selected from (C₆ to C₁₀) aryl and (C₂ to C₉) heteroaryl, wherein
each of the said (C₆ to C₁₀) aryl and (C₂ to C₉) heteroaryl is optionally substituted with at least one R⁸ group;
and when Q is CH₂, then
R² is selected from hydrogen, halogen, OH, nitro, CF₃, —NR^{6a}R^{6b}, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, —C(O)R⁷, —C(O)NR^{6a}R^{6b}, —S(O)ₘR⁷, —S(O)ₘNR^{6a}R^{8b}, —NR^{6a}S(O)ₘR⁷, —(CH₂)ₙC(O)OR⁷, —(CH₂)ₙC(O)N(R^{6a}R^{6b}), —(CH₂)ₙN(R^{6a}R^{6b}), —OC(O)R⁷, —NR^{6a}C(O)R⁷, and —NR^{6a}C(O)N(R^{6a}R^{6b}), wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁸ group;
and when Q is CR²³R²⁴ then R² is hydrogen;
NR^{3a}R^{3b} is selected from the group consisting of

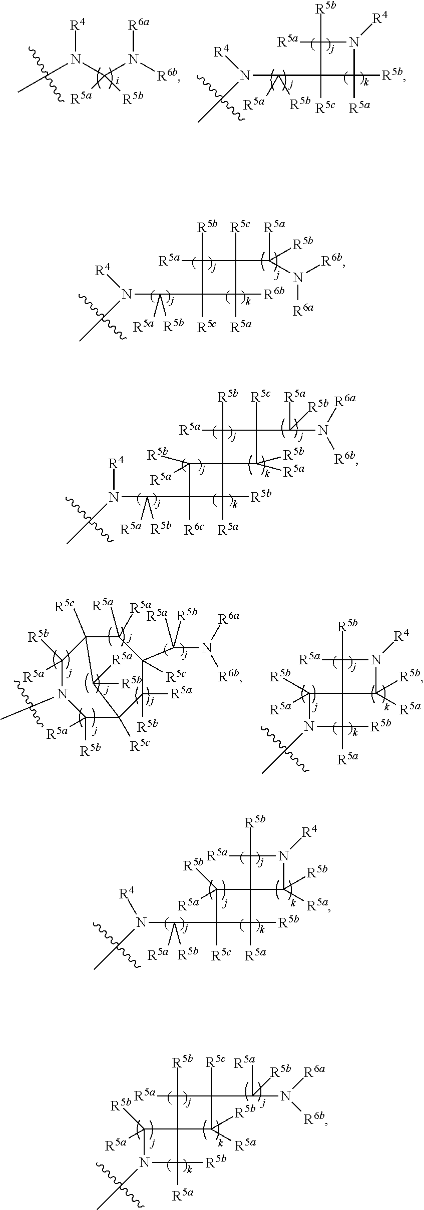

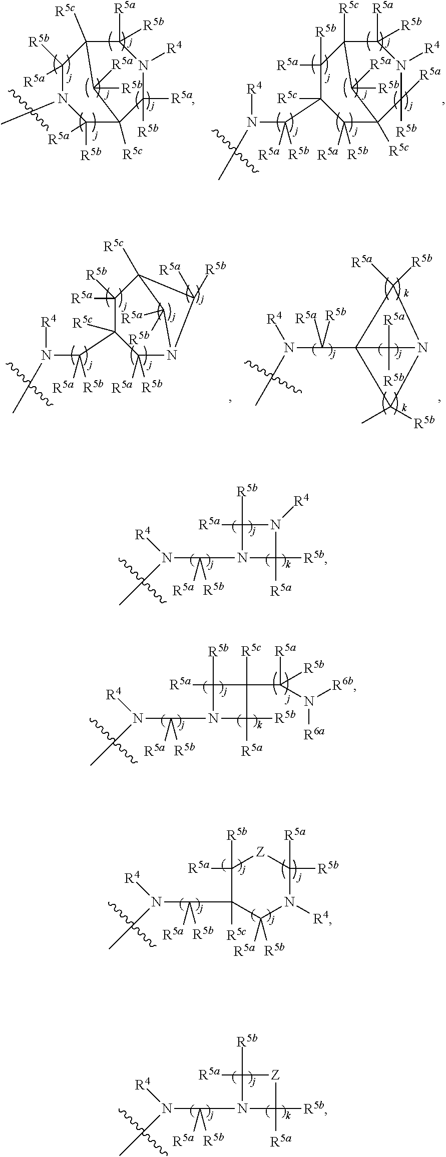

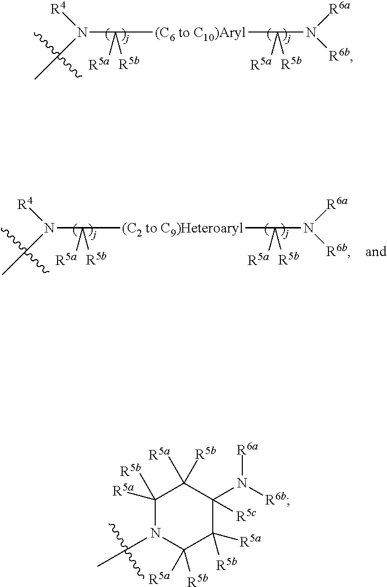

Z is selected from the group consisting of —O—, —S—, —S(O)—, and —S(O)₂—;
each R⁴ is independently selected from hydrogen, (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C to C₁₀) cycloalkenyl, (C₂ to C₉) cycloheteroalkyl, (C₆ to C₁₀) aryl, (C₂ to C₉) heteroaryl, (C₆ to C₁₀) arylene, and (C₂ to C₉) heteroarylene, wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, (C₆ to C₁₀) arylene, and (C₂ to C₉) heteroarylene is optionally substituted with at least one R⁸ group;
each of the R^{5a}, R^{5b}, and R^{5c} is independently selected from hydrogen, halogen, OH, nitro, CF₃, —NR^{6a}R^{6b}, (C₁ to C₁₀) alkyl, (C₁ to C₁₀) alkenyl, (C₁ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, —C(O)R⁷, —C(O)NR^{6a}R^{6b}, —S(O)ₘR⁷, —S(O)ₘNR^{6a}R^{6b}, —NR^{6a}S(O)ₘR⁷, —(CH₂)ₙC(O)OR⁷, —(CH₂)ₙC(O)N(R^{6a}R^{6b}), —(CH₂)ₙN(R^{6a}R^{6b}), —OC(O)R⁷, —NR^{6a}C(O)R⁷, and —NR^{6a}C(O)N(R^{6a}R^{6b}), wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁸ group;
or R^{5a} and R^{5b} may be taken together with the carbon atom to which they are attached to form a (C₃ to C₁₀) cycloalkyl ring, wherein
the said (C₃ to C₁₀) cycloalkyl ring is optionally substituted with at least one R⁸ group;
each of the R^{6a} and R^{6b} is independently selected from hydrogen, (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, (C₆ to C₁₀) arylene, and (C₂ to C₉) heteroarylene, wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, (C₆ to C₁₀) arylene, and (C₂ to C₉) heteroarylene is optionally substituted with at least one R⁸ group,
or R^{6a} and R^{6b} may be taken together with the nitrogen atom to which they are attached to form a (C₂ to C₁₀) cycloheteroalkyl ring, wherein
said (C₂ to C₁₀) cycloheteroalkyl ring has 1 to 3 ring heteroatoms selected from the group consisting of N, O, and S, and wherein
the said (C₂ to C₁₀) cycloheteroalkyl ring is optionally substituted with at least one R⁸ group;
each of the R⁷ is independently selected from hydrogen, (C₁ to C₁₀) alkyl, (C₂ to C₁₀) alkenyl, (C₂ to C₁₀) alkynyl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl, wherein
   each of the said (C₁ to C₁₀) alkyl, (C₂ to C₁₀) alkenyl, (C₂ to C₁₀) alkynyl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁸ group;
each R⁸ is independently selected from hydrogen, halogen, OH, nitro, CF₃, —NR^{9a}R^{9b}, oxo, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, (C₆ to C₁₀) arylene, (C₂ to C₉) heteroarylene, (C₃ to C₁₀) cycloalkylene, (C₂ to C₁₀) cycloheteroalkylene, —C(O)R¹⁰, —C(O)NR^{9a}R^{9b}, —S(O)ₘR¹⁰, —S(O)ₘNR^{9a}R^{9b}, —NR^{9a}S(O)ₘR¹⁰, —(CH₂)ₙC(O)OR¹⁰, —(CH₂)ₙC(O)N(R^{9a}R^{9b}), —(CH₂)ₙN(R^{9a}R^{9b}), —OC(O)R¹⁵, —O(CH₂)ₙO—, —NR^{9a}C(O)R^{1′}, and —NR^{9a}C(O)N(R^{9a}R^{9b}), wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, (C₂ to C₉) heteroarylene, (C₃ to C₁₀) cycloalkylene, and (C₂ to C₁₀) cycloheteroalkylene is optionally substituted with at least one R¹¹ group;
each of the R^{9a} and R^{9b} is independently selected from hydrogen, (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, (C₆ to C₁₀) arylene, and (C₂ to C₉) heteroarylene, wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, and (C₂ to C₉) heteroarylene is optionally substituted with at least one R¹¹ group,
or R^{9a} and R^{9b} may be taken together with the nitrogen atom to which they are attached to form a (C₂ to C₁₀) cycloheteroalkyl ring, wherein
said (C₂ to C₁₀) cycloheteroalkyl ring has 1 to 3 ring heteroatoms selected from the group consisting of N, O, and S, and wherein
the said (C₂ to C₁₀) cycloheteroalkyl ring is optionally substituted with at least one R¹ group;
each R¹⁰ is independently selected from hydrogen, (C₁ to C₁₀) alkyl, (C₂ to C₁₀) alkenyl, (C₂ to C₁₀) alkynyl, (C₃ to C₁₀) cycloalkyl, (C₅ to C₁₀) cycloalkenyl, (C₂ to C₁₀) cycloheteroalkyl, (C₆ to C₁₀) aryl, and (C₂ to C₉) heteroaryl, wherein
each of the said (C₁ to C₁₀) alkyl, (C₂ to C₁₀) alkenyl, (C₂ to C₁₀) alkynyl, (C₃ to C₁₀) cycloalkyl, (C₅ to C₁₀) cycloalkenyl, (C₂ to C₁₀) cycloheteroalkyl, (C₆ to C₁₀) aryl, and (C₂ to C₉) heteroaryl is optionally substituted with at least one R¹¹ group;
each R¹¹ is independently selected from hydrogen, halogen, OH, nitro, CF₃, —NR^{12a}R^{12b}, oxo, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, (C₂ to C₉) heteroarylene, (C₃ to C₁₀) cycloalkylene, (C₂ to C₁₀) cycloheteroalkylene, —C(O)R¹⁸, —C(O)NR^{12a}R^{12b}, —S(O)ₘR¹³, —S(O)ₘNR^{12a}R^{12b}, —NR^{12a}S(O)ₘR¹³, —(CH₂)ₙC(O)OR¹³, —(CH₂)ₙC(O)N(R^{12a}R^{12b}), —(CH₂)ₙN(R^{12a}R^{12b}), —OC(O)R¹³, —NR^{12a}C(O)R¹³, and —NR^{12a}C(O)N(R^{12a}R^{12b}), wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₂ to C₉) heteroaryl, (C₆ to C₁₀) aryl, (C₆ to C₁₀) arylene, (C₂ to C₉) heteroarylene, (C₃ to C₁₀) cycloalkylene, and (C₂ to C₁₀) cycloheteroalkylene is optionally substituted with at least one R¹⁴ group;
each of the R^{12a} and R^{12b} is independently selected from hydrogen, (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, and (C₂ to C₉) heteroarylene, wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, and (C₂ to C₉) heteroarylene is optionally substituted with at least one R¹⁴ group,
or R^{12a} and R^{12b} may be taken together with the nitrogen atom to which they are attached to form a (C₂ to C₁₀) cycloheteroalkyl ring, wherein
said (C₂ to C₁₀) cycloheteroalkyl ring has 1 to 3 ring heteroatoms selected from the group consisting of N, O, and S, and wherein
the said (C₂ to C₁₀) cycloheteroalkyl ring is optionally substituted with at least one R¹⁴ group;
each R¹³ is independently selected from hydrogen, halogen, OH, nitro, CF₃, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₁₀) cycloheteroalkyl, (C₆ to C₁₀) aryl, and (C₂ to C₉) heteroaryl, wherein
each of the said (C₁ to C₁₀) alkyl, (C₂ to C₁₀) alkenyl, (C₂ to C₁₀) alkynyl, (C₃ to C₁₀) cycloalkyl, (C₅ to C₁₀) cycloalkenyl, (C₂ to C₁₀) cycloheteroalkyl, (C₂ to C₉) heteroaryl, and (C₆ to C₁₀) aryl is optionally substituted with at least one R¹⁴ group;
each R¹⁴ is independently selected from hydrogen, halogen, OH, nitro, CF₃, —NR^{15a}R^{15b}, oxo, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, (C₂ to C₉) heteroarylene, (C₃ to C₁₀) cycloalkylene, (C₂ to C₁₀) cycloheteroalkylene, —C(O)R¹⁶, —C(O)NR^{15a}R^{15b}—S(O)ₘR¹⁶, —S(O)ₘNR^{15a}R^{15b}, —NR^{15a}S(O)ₘR¹⁶, —(CH₂)ₙC(O)OR¹⁶, —(CH₂)ₙC(O)N(R^{15a}R^{15b}), —(CH₂)ₙN(R^{15a}R^{15b}), —OC(O)R¹⁶, —NR^{15a}C(O)R¹⁶, and —NR^{15a}C(O)N(R^{15a}R^{15b}), wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, (C₂ to C₉) heteroarylene, (C₃ to C₁₀) cycloalkylene, and (C₂ to C₁₀) cycloheteroalkylene is optionally substituted with at least one R¹⁷ group;
each of the R^{15a} and R^{15b} is independently selected from hydrogen, (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, and (C₂ to C₉) heteroaryl, wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, and (C₂ to C₉) heteroaryl is optionally substituted with at least one R¹⁷ group,
or R^{15a} and R^{15b} may be taken together with the nitrogen atom to which they are attached to form a (C₂ to C₁₀) cycloheteroalkyl ring, wherein
said (C₂ to C₁₀) cycloheteroalkyl ring has 1 to 3 ring heteroatoms selected from the group consisting of N, O, and S, and wherein
the said (C₂ to C₁₀) cycloheteroalkyl ring is optionally substituted with at least one R¹⁷ group;
each R¹⁶ is independently selected from hydrogen, halogen, OH, nitro, CF₃, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₁₀) cycloheteroalkyl, (C₆ to C₁₀) aryl, and (C₂ to C₉) heteroaryl;
each R¹⁷ is independently selected from hydrogen, halogen, OH, nitro, CF₃, —NR^{18a}R^{18b}, oxo, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, (C₂ to C₉) heteroarylene, (C₃ to C₁₀) cycloalkylene, (C₂ to C₁₀) cycloheteroalkylene, —C(O)R¹⁹, —C(O)NR^{13a}R^{18b}, —S(O)ₘR¹⁹, —S(O)ₘNR^{13a}R^{18b}, —NR^{18a}S(O)ₘR¹⁹, —(CH₂)ₙC(O)OR¹⁹, —(CH₂)ₙC(O)N(R^{18a}R^{18b}), —(CH₂)ₙN(R^{18a}R^{18b}), —OC(O)R¹⁹, —NR^{18a}C(O)R¹⁹, and —NR^{18a}C(O)N(R^{18a}R^{18b}), wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, (C₂ to C₉) heteroarylene, (C₃ to C₁₀) cycloalkylene, and (C₂ to C₁₀) cycloheteroalkylene is optionally substituted with at least one R²⁰ group;
each of the R^{18a} and R^{18b} is independently selected from hydrogen, (C₁ to C₁₀) alkyl, (C₂ to C₁₀) alkenyl, (C₂ to C₁₀) alkynyl, (C₃ to C₁₀) cycloalkyl, (C₅ to C₁₀) cycloalkenyl, (C₂ to C₁₀) cycloheteroalkyl, (C₂ to C₉) heteroaryl, and (C₆ to C₁₀) aryl;
each R¹⁹ is independently selected from hydrogen, halogen, OH, nitro, CF₃, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl;
each R²⁰ is independently selected from hydrogen, halogen, OH, nitro, CF₃, —NR^{21a}R^{21b}, oxo, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, (C₂ to C₉) heteroarylene, (C₃ to C₁₀) cycloalkylene, (C₂ to C₁₀) cycloheteroalkylene, —C(O)R²², —C(O)NR^{21a}R^{21b}, —S(O)ₘR²², —S(O)ₘNR^{21a}R^{21b}, —NR^{21a}S(O)ₘR²², —(CH₂)ₙC(O)OR²², —(CH₂)ₙC(O)N(R^{21a}R^{21b}), —(CH₂)ₙN(R^{21a}R^{21b}), —OC(O)R²², —NR^{21a}C(O)R²², and —NR^{21a}C(O)N(R^{21a}R^{21b}), wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, (C₂ to C₉) heteroarylene, (C₃ to C₁₀) cycloalkylene, and (C₂ to C₁₀) cycloheteroalkylene is optionally substituted with at least one R²² group;
each of the R^{21a} and R^{21b} is independently selected from hydrogen, (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, and (C₂ to C₉) heteroaryl,
or R^{21a} and R^{21b} may be taken together with the nitrogen atom to which they are attached to form a (C₂ to C₁₀) cycloheteroalkyl ring, wherein
said (C₂ to C₁₀) cycloheteroalkyl ring has 1 to 3 ring heteroatoms selected from the group consisting of N, O, and S;
each R²² is independently selected from hydrogen, halogen, OH, nitro, CF₃, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl; each R²³ is independently selected from hydrogen, halogen, OH, nitro, CF₃, —NR^{11a}R^{11b}, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cyclo-alkenyl, (C₂ to C₉) cycloheteroalkyl, (C₆ to C₁₀) aryl, (C₂ to C₉) heteroaryl, —C(O)R¹², —C(O)NR^{11a}R^{11b}, —S(O)ₘR¹², —S(O)ₘNR^{11a}R^{11b}, —NR^{11a}S(O)ₘR¹², —(CH₂)ₙC(O)OR¹², —(CH₂)ₙC(O)N(R^{11a}R^{11b}), —(CH₂)ₙN(R^{11a}R^{11b}), —OC(O)R¹², —NR^{11a}C(O)R¹², and —NR^{11a}C(O)N(R^{11a}R^{11b}) wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁸ group;
each R²⁴ is independently selected from halogen, OH, nitro, CF₃, —NR^{11a}R^{11b}, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, —C(O)R¹², —C(O)NR^{11a}R^{11b}, —S(O)ₘR¹², —S(O)ₘNR^{11a}R^{11b}, —NR^{11a}S(O)ₘR¹², —(CH₂)ₙC(O)OR¹², —(CH₂)ₙC(O)N(R^{11a}R^{11b}), —(CH₂)ₙN(R^{11a}R^{11b}), —OC(O)R¹², —NR^{11a}C(O)R¹², and —NR^{11a}C(O)N(R^{11a}R^{11b}) wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁸ group;
or R²³ and R²⁴ may be taken together with the carbon atom to which they are attached to form a (C₃ to C₁₀) cycloalkyl or (C₂ to C₉) cycloheteroalkyl ring, wherein
the said (C₃ to C₁₀) cycloalkyl or (02 to 09) cycloheteroalkyl ring is optionally substituted with at least one R⁸ group;
or R²³ and R²⁴ may be taken together with the carbon atom to which they are attached to form a carbonyl or alkene, which is optionally substituted with at least one R⁸ group;
i is 2, 3, 4, 5, or 6;
j is 0, 1, 2, 3, 4, or 5;
k is 1, 2, 3, 4, or 5;
m is 0, 1 or 2;
n is 0, 1, 2, 3, or 4;
with the proviso that when R¹ is phenyl, R² is hydrogen, X is

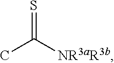

Y is CH₂, and Q is CH₂, then NR^{3a}R^{3b} is not

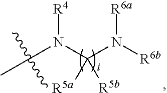

and with the proviso that the following compounds are excluded:

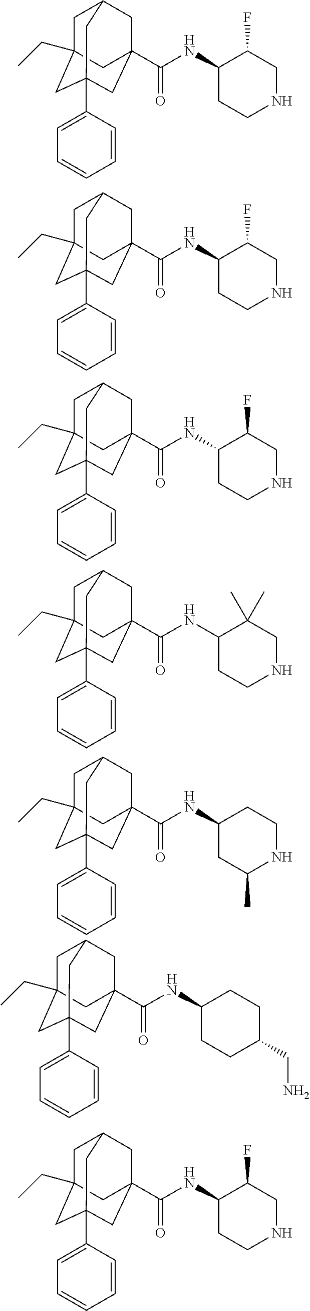

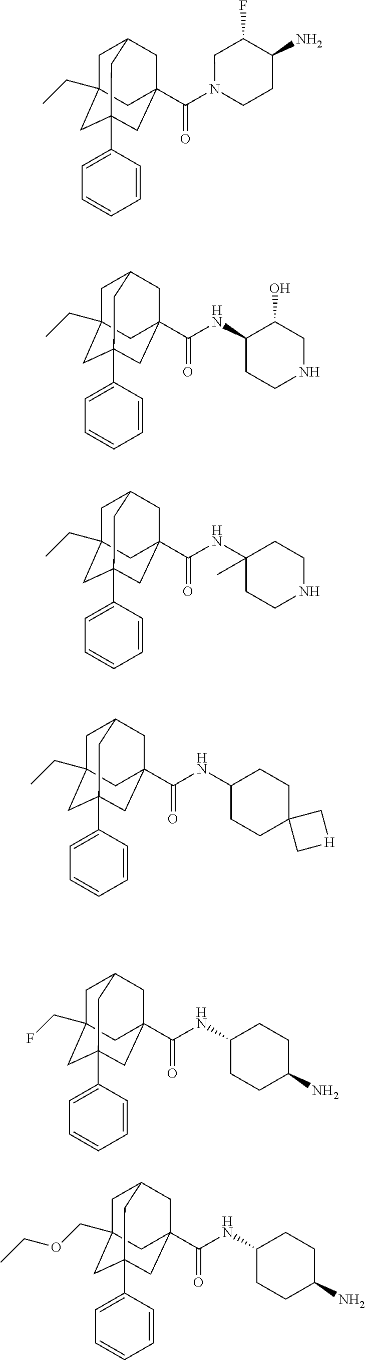

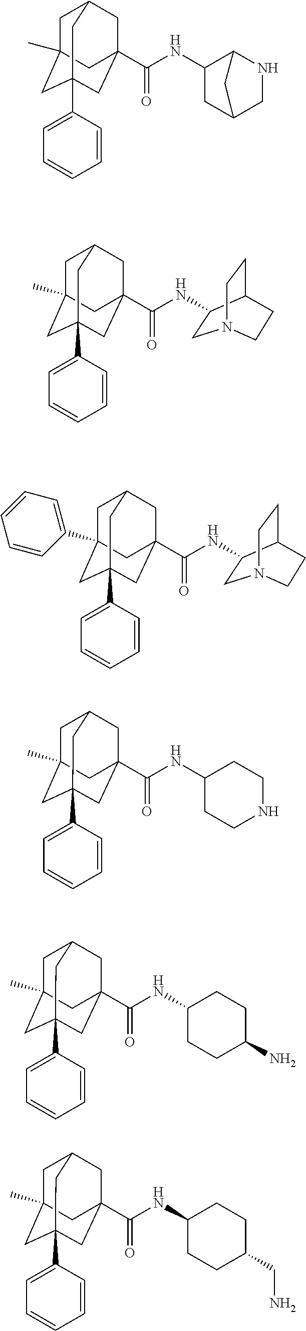

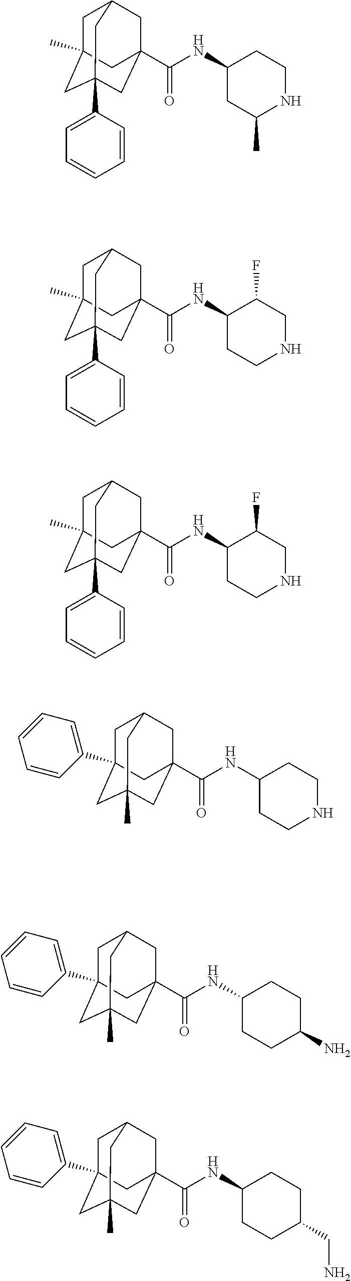 [SMILES: C[C@H]1C[C@@H](NC(=O)C23CC4C[C@@](C)(C2)C[C@](c2ccccc2)(C4)C3)CCN1 ; C[C@]12CC3CC(C(=O)N[C@@H]4CCNC[C@H]4F)(C1)C[C@@](c1ccccc1)(C3)C2 ; C[C@]12CC3CC(C(=O)N[C@@H]4CCNC[C@@H]4F)(C1)C[C@@](c1ccccc1)(C3)C2 ; C[C@@]12CC3CC(C(=O)NC4CCNCC4)(C1)C[C@](c1ccccc1)(C3)C2 ; C[C@@]12CC3CC(C(=O)N[C@H]4CC[C@H](N)CC4)(C1)C[C@](c1ccccc1)(C3)C2 ; C[C@@]12CC3CC(C(=O)N[C@H]4CC[C@H](CN)CC4)(C1)C[C@](c1ccccc1)(C3)C2]

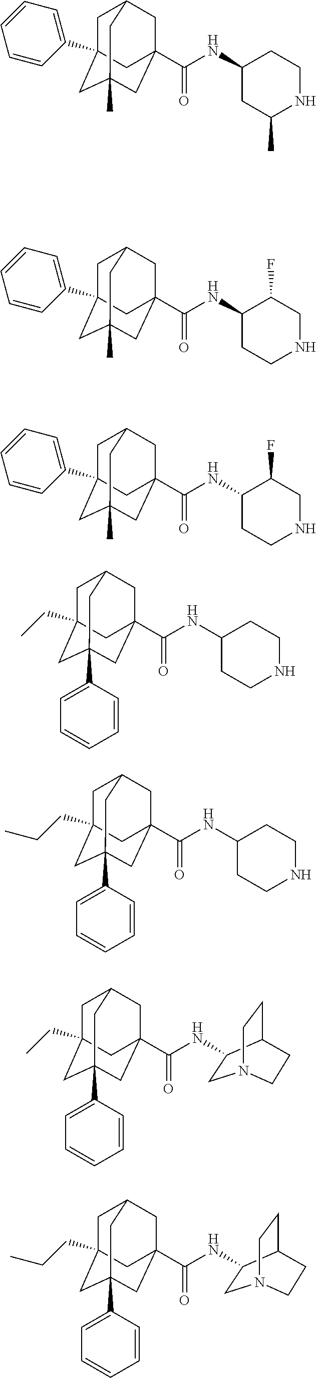

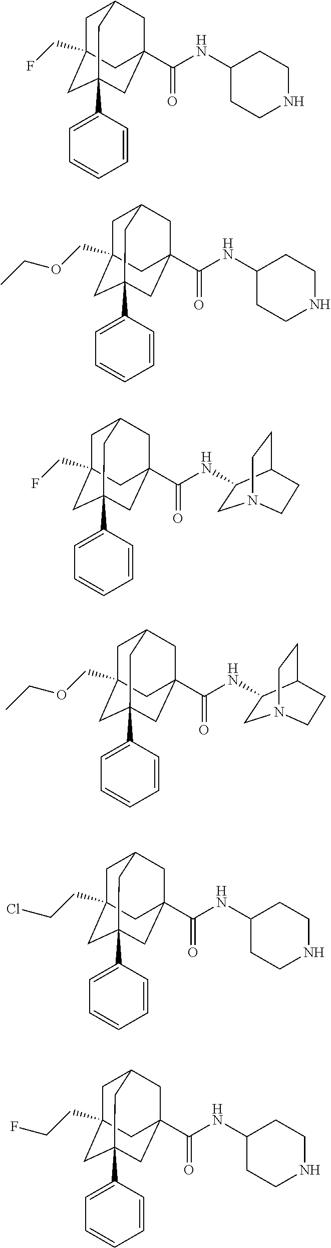

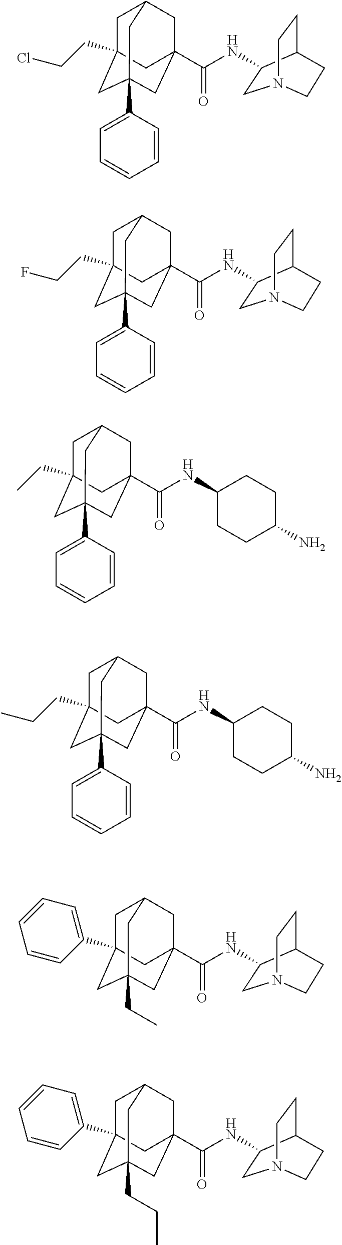

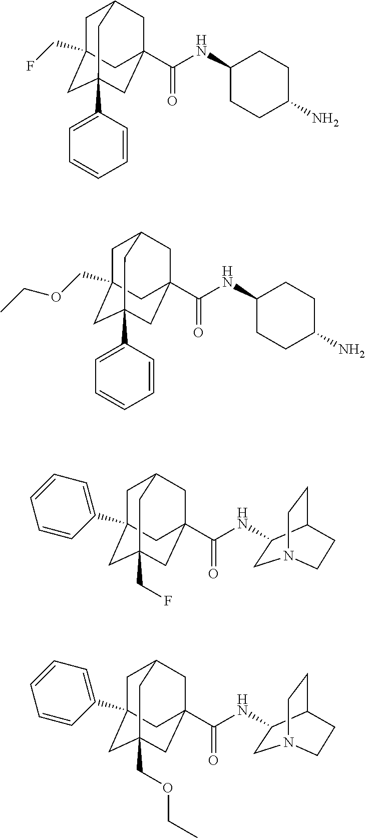 [SMILES: N[C@H]1CC[C@H](NC(=O)C23CC4C[C@@](CF)(C2)C[C@](c2ccccc2)(C4)C3)CC1 ; CCOC[C@]12CC3CC(C(=O)N[C@H]4CC[C@H](N)CC4)(C1)C[C@@](c1ccccc1)(C3)C2 ; O=C(N[C@H]1CN2CCC1CC2)C12CC3C[C@](CF)(C1)C[C@@](c1ccccc1)(C3)C2 ; CCOC[C@@]12CC3CC(C(=O)N[C@H]4CN5CCC4CC5)(C1)C[C@](c1ccccc1)(C3)C2]

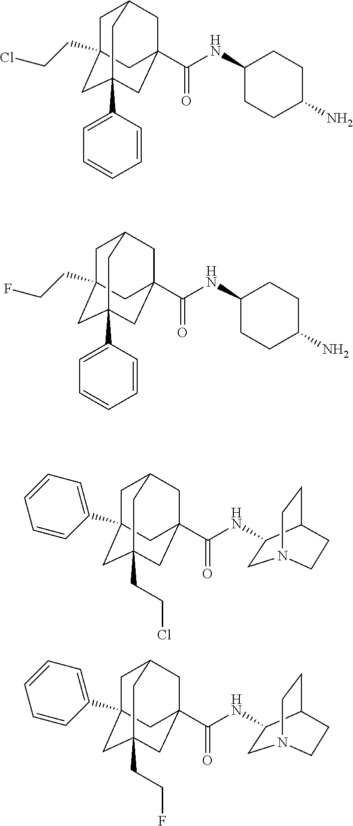

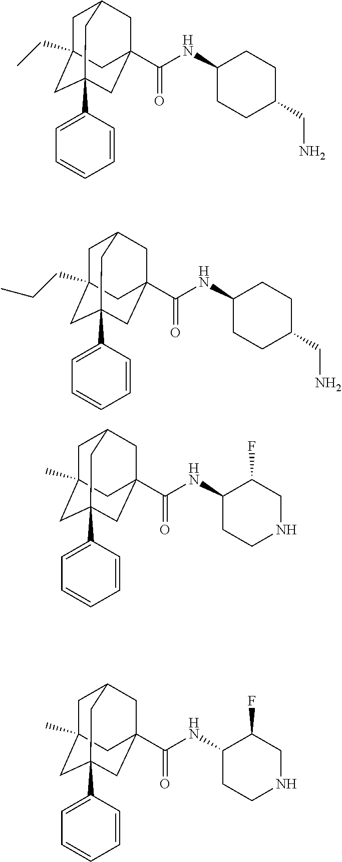

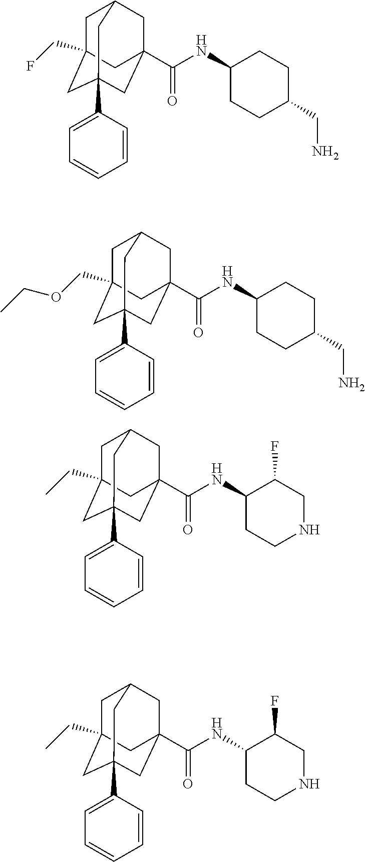

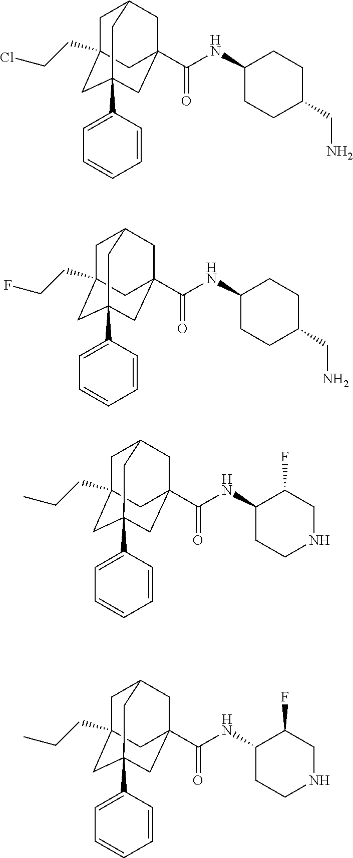 [SMILES: NC[C@H]1CC[C@H](NC(=O)C23CC4C[C@@](CCCl)(C2)C[C@](c2ccccc2)(C4)C3)CC1 ; NC[C@H]1CC[C@H](NC(=O)C23CC4C[C@@](CCF)(C2)C[C@](c2ccccc2)(C4)C3)CC1 ; CCC[C@]12CC3CC(C(=O)N[C@@H]4CCNC[C@H]4F)(C1)C[C@@](c1ccccc1)(C3)C2 ; CCC[C@]12CC3CC(C(=O)N[C@H]4CCNC[C@@H]4F)(C1)C[C@@](c1ccccc1)(C3)C2]

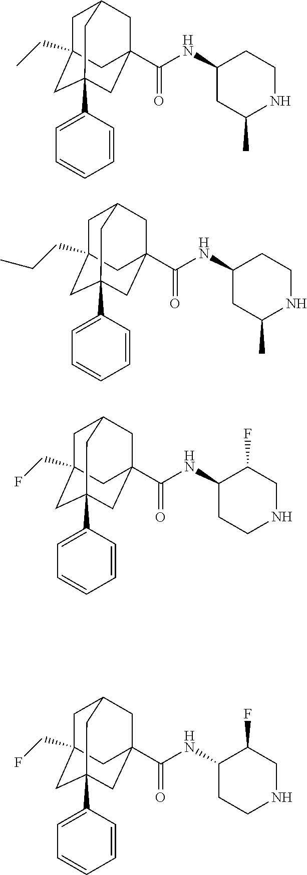

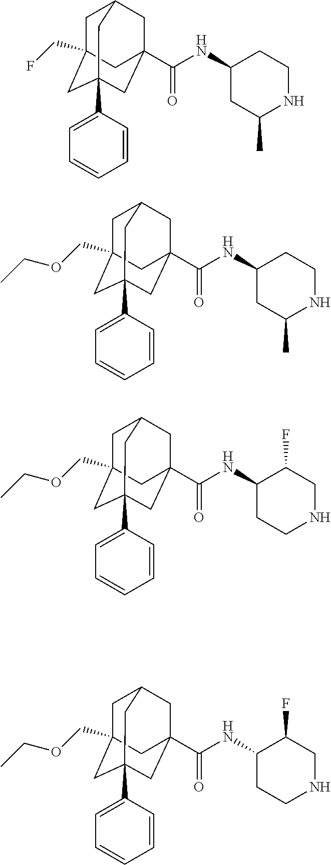

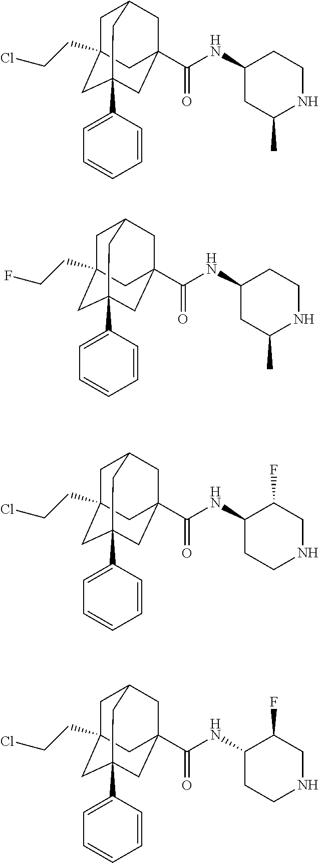

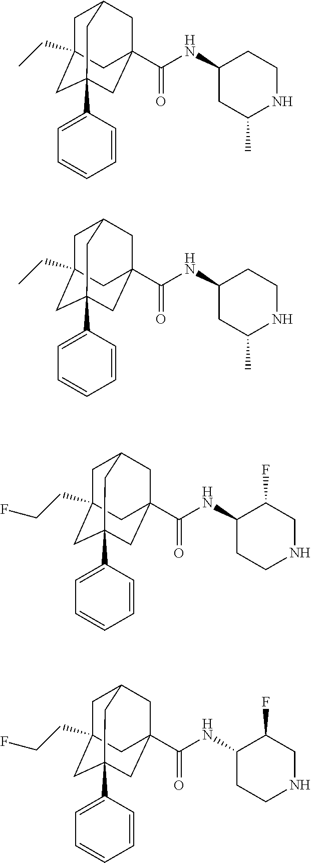

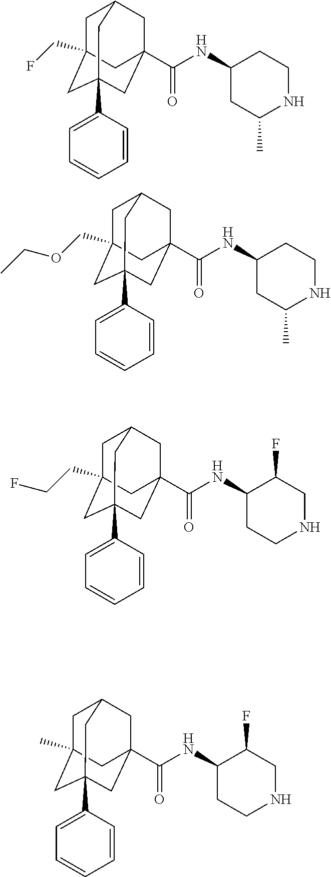

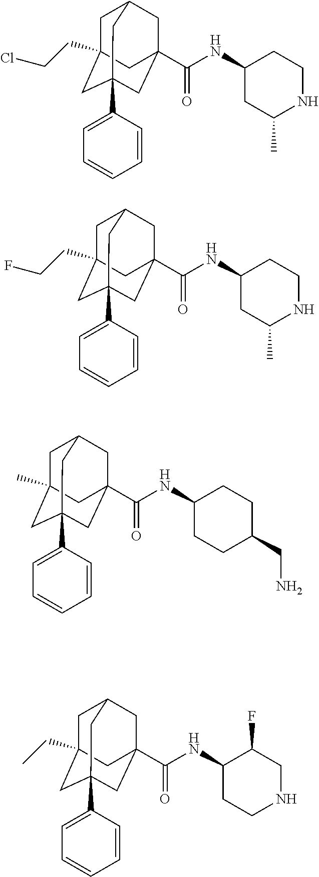

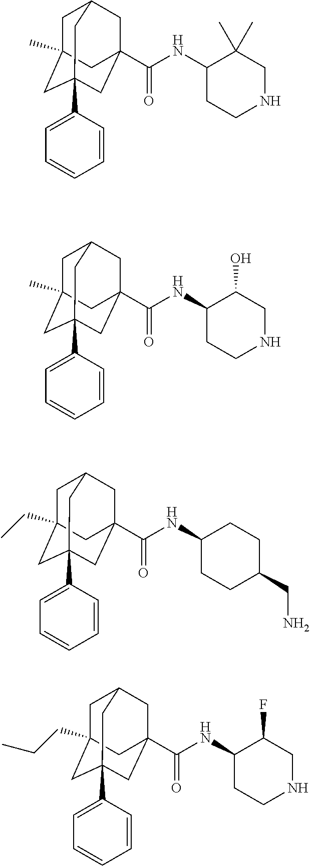 [SMILES: CC1(C)CNCCC1NC(=O)C12CC3C[C@@](C)(C1)C[C@](c1ccccc1)(C3)C2 ; C[C@]12CC3CC(C(=O)N[C@@H]4CCNC[C@H]4O)(C1)C[C@@](c1ccccc1)(C3)C2 ; CC[C@]12CC3CC(C(=O)N[C@H]4CC[C@@H](CN)CC4)(C1)C[C@@](c1ccccc1)(C3)C2 ; CCC[C@]12CC3CC(C(=O)N[C@@H]4CCNC[C@@H]4F)(C1)C[C@@](c1ccccc1)(C3)C2]

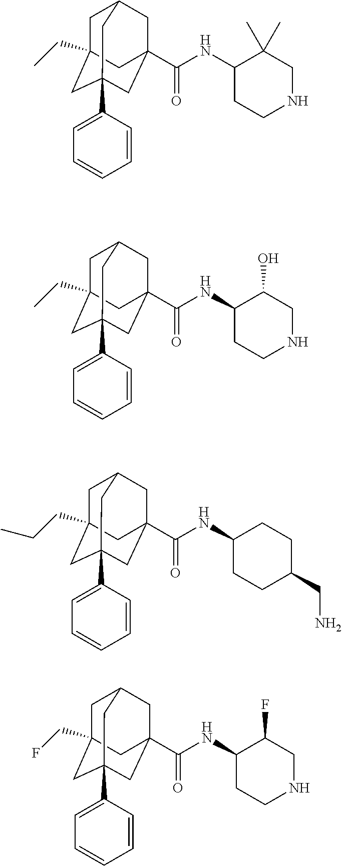

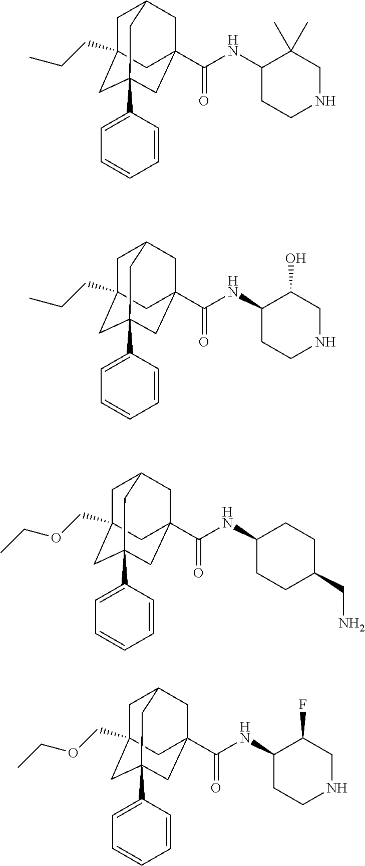 [SMILES: CCC[C@]12CC3CC(C(=O)NC4CCNCC4(C)C)(C1)C[C@@](c1ccccc1)(C3)C2 ; CCC[C@]12CC3CC(C(=O)N[C@@H]4CCNC[C@H]4O)(C1)C[C@@](c1ccccc1)(C3)C2 ; CCOC[C@]12CC3CC(C(=O)N[C@H]4CC[C@@H](CN)CC4)(C1)C[C@@](c1ccccc1)(C3)C2 ; CCOC[C@]12CC3CC(C(=O)N[C@@H]4CCNC[C@@H]4F)(C1)C[C@@](c1ccccc1)(C3)C2]

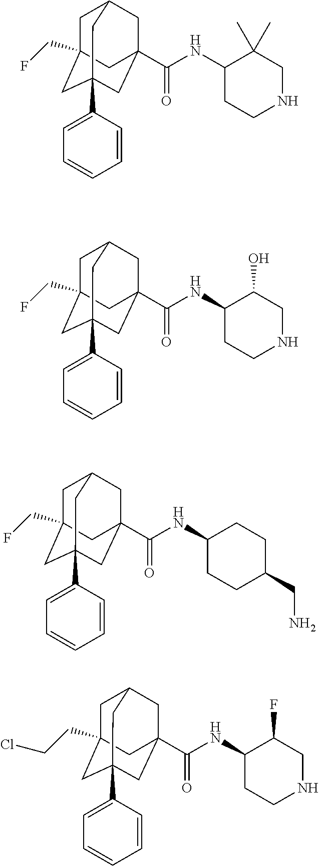 [SMILES: CC1(C)CNCCC1NC(=O)C12CC3C[C@@](CF)(C1)C[C@](c1ccccc1)(C3)C2 ; O=C(N[C@@H]1CCNC[C@H]1O)C12CC3C[C@@](CF)(C1)C[C@](c1ccccc1)(C3)C2 ; NC[C@H]1CC[C@@H](NC(=O)C23CC4C[C@@](CF)(C2)C[C@](c2ccccc2)(C4)C3)CC1 ; O=C(N[C@@H]1CCNC[C@@H]1F)C12CC3C[C@@](CCCl)(C1)C[C@](c1ccccc1)(C3)C2]

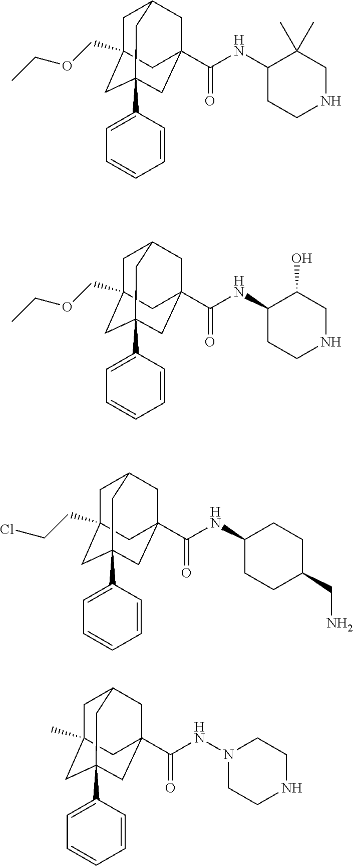

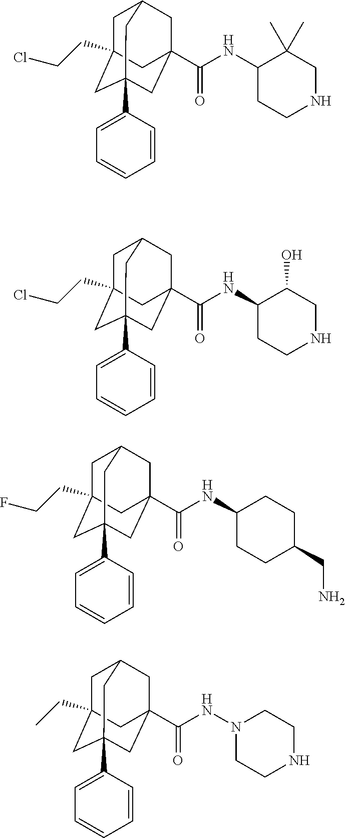

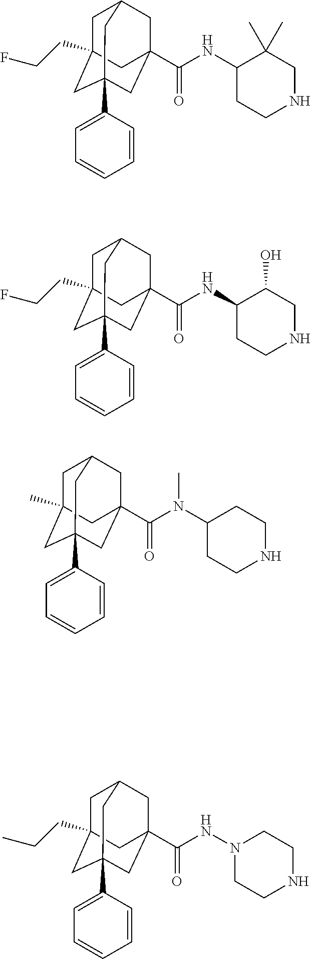

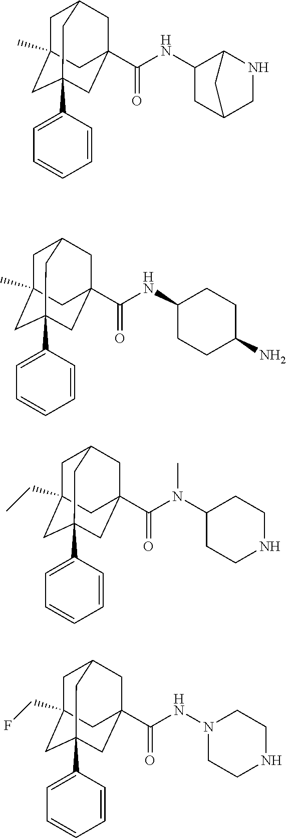

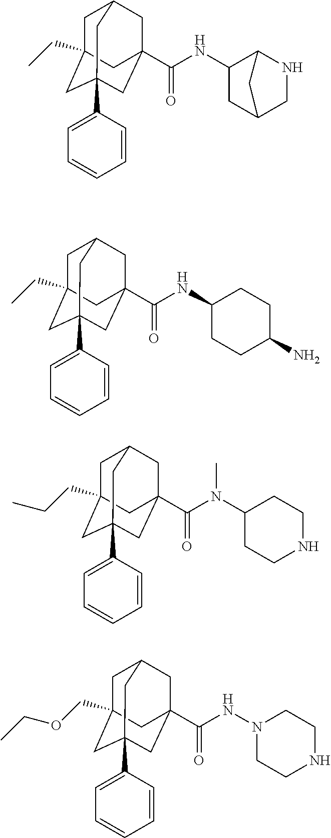 [SMILES: CC[C@]12CC3CC(C(=O)NC4CC5CNC4C5)(C1)C[C@@](c1ccccc1)(C3)C2 ; CC[C@]12CC3CC(C(=O)N[C@H]4CC[C@@H](N)CC4)(C1)C[C@@](c1ccccc1)(C3)C2 ; CCC[C@]12CC3CC(C(=O)N(C)C4CCNCC4)(C1)C[C@@](c1ccccc1)(C3)C2 ; CCOC[C@]12CC3CC(C(=O)NN4CCNCC4)(C1)C[C@@](c1ccccc1)(C3)C2]

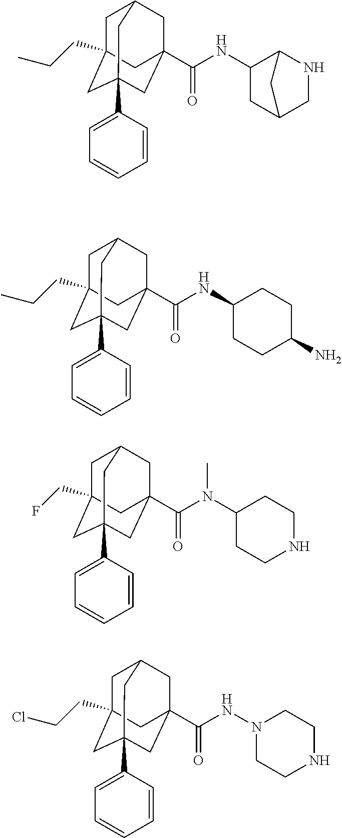

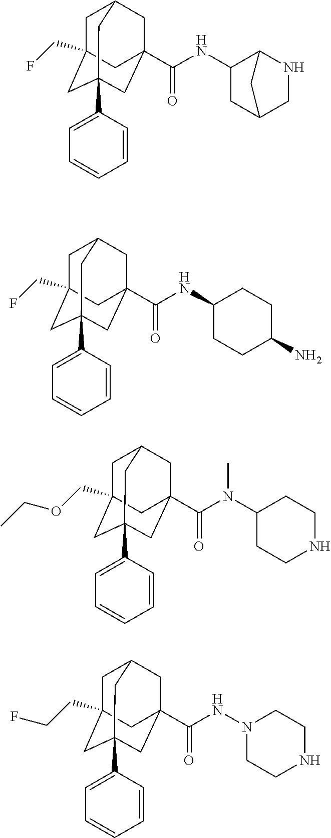

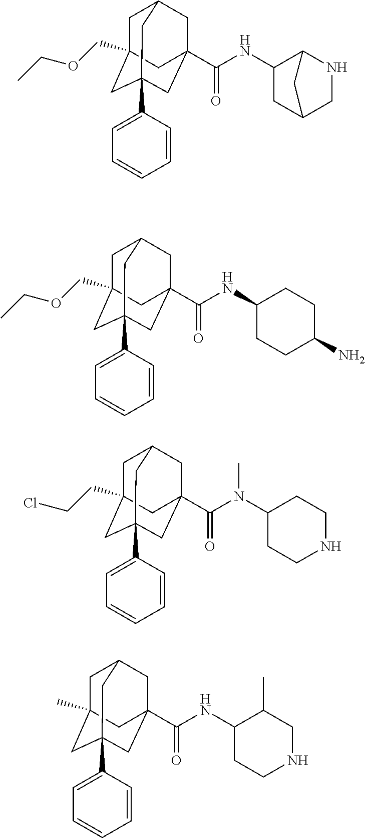

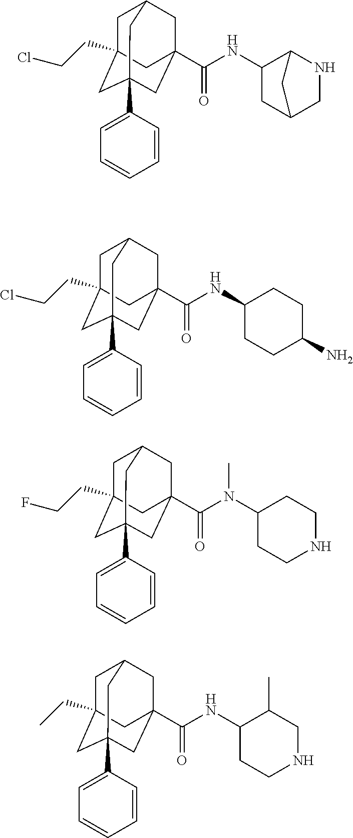

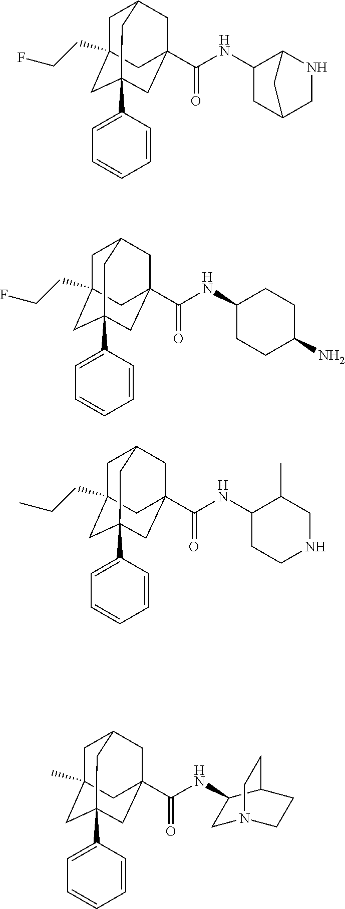

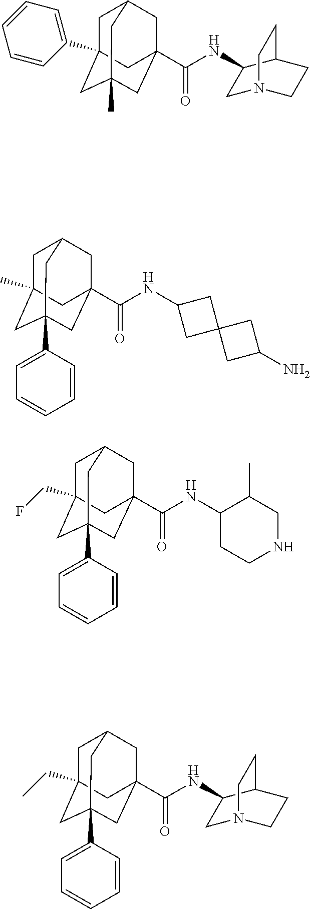 [SMILES: C[C@@]12CC3CC(C(=O)N[C@@H]4CN5CCC4CC5)(C1)C[C@](c1ccccc1)(C3)C2 ; C[C@]12CC3CC(C(=O)NC4CC5(CC(N)C5)C4)(C1)C[C@@](c1ccccc1)(C3)C2 ; CC1CNCCC1NC(=O)C12CC3C[C@@](CF)(C1)C[C@](c1ccccc1)(C3)C2 ; CC[C@]12CC3CC(C(=O)N[C@@H]4CN5CCC4CC5)(C1)C[C@@](c1ccccc1)(C3)C2]

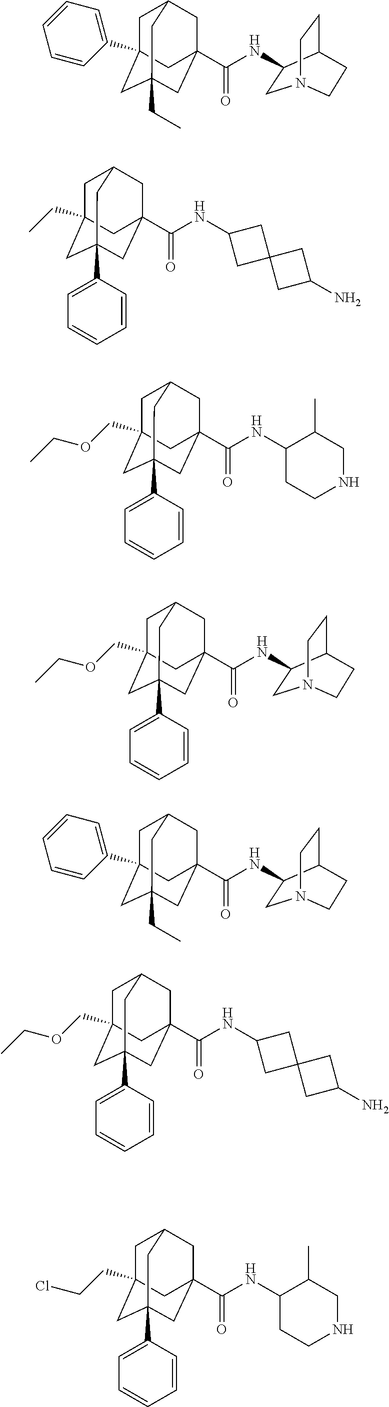

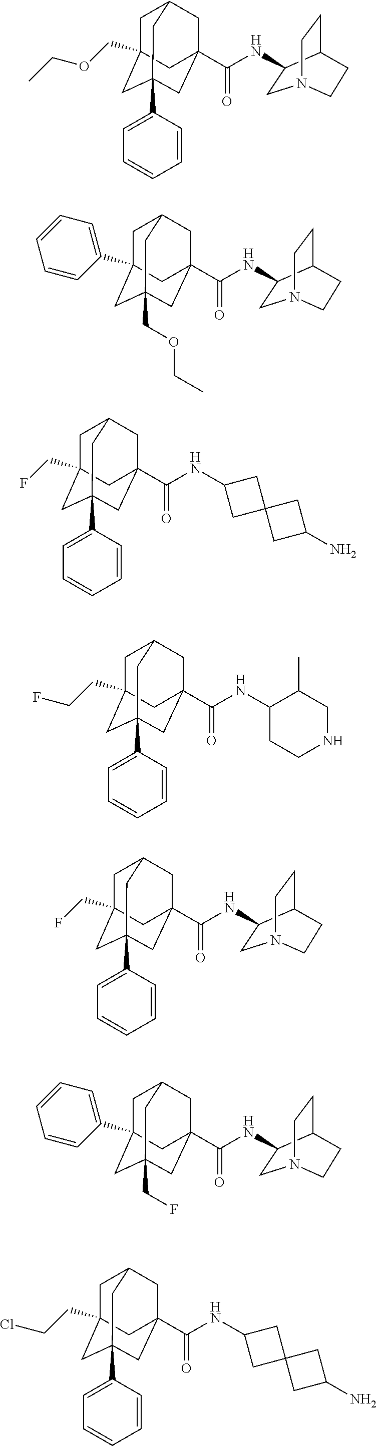

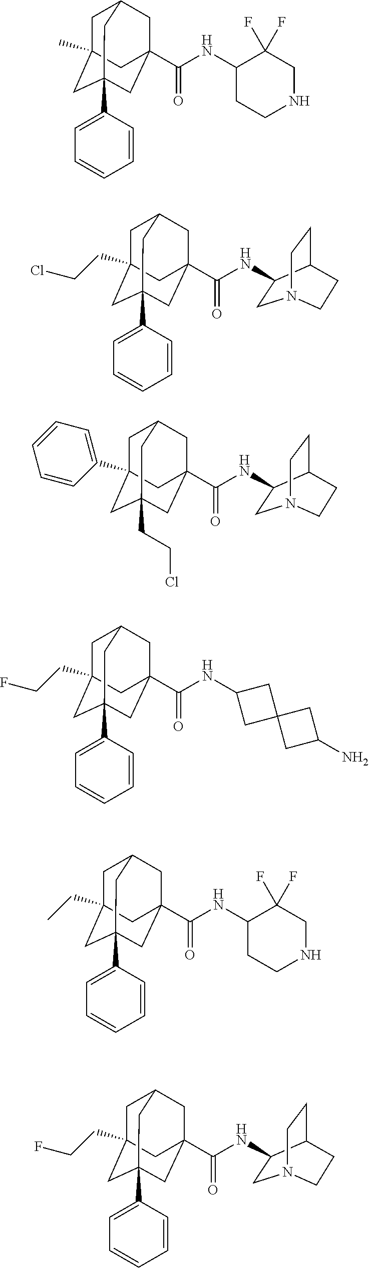

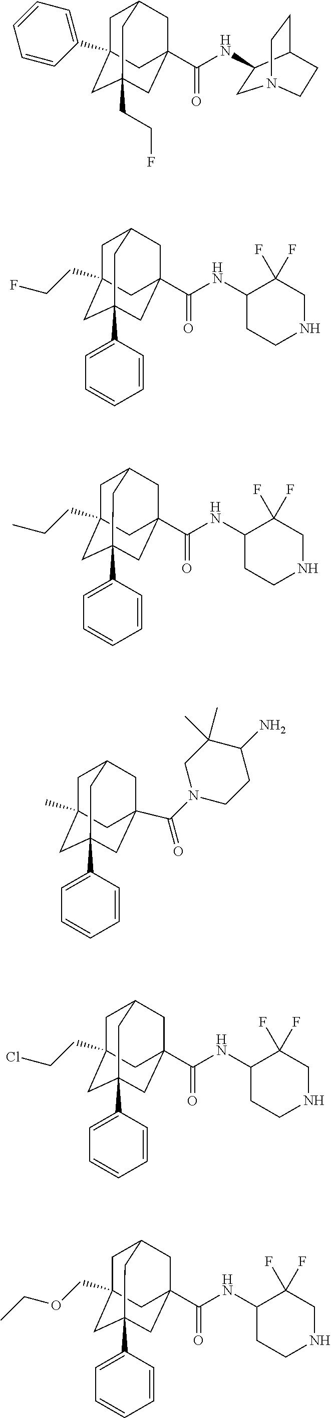 [SMILES: O=C(N[C@@H]1CN2CCC1CC2)C12CC3C[C@](CCF)(C1)C[C@@](c1ccccc1)(C3)C2 ; O=C(NC1CCNCC1(F)F)C12CC3C[C@@](CCF)(C1)C[C@](c1ccccc1)(C3)C2 ; CCC[C@]12CC3CC(C(=O)NC4CCNCC4(F)F)(C1)C[C@@](c1ccccc1)(C3)C2 ; CC1(C)CN(C(=O)C23CC4C[C@@](C)(C2)C[C@](c2ccccc2)(C4)C3)CCC1N ; O=C(NC1CCNCC1(F)F)C12CC3C[C@@](CCCl)(C1)C[C@](c1ccccc1)(C3)C2 ; CCOC[C@]12CC3CC(C(=O)NC4CCNCC4(F)F)(C1)C[C@@](c1ccccc1)(C3)C2]

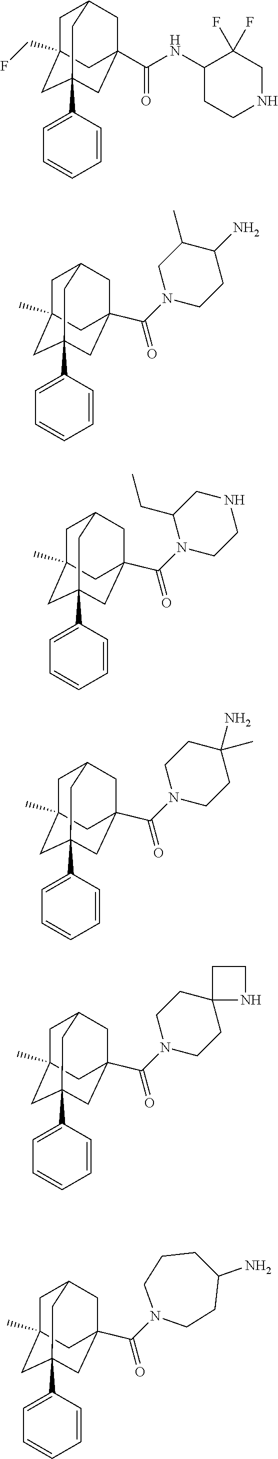

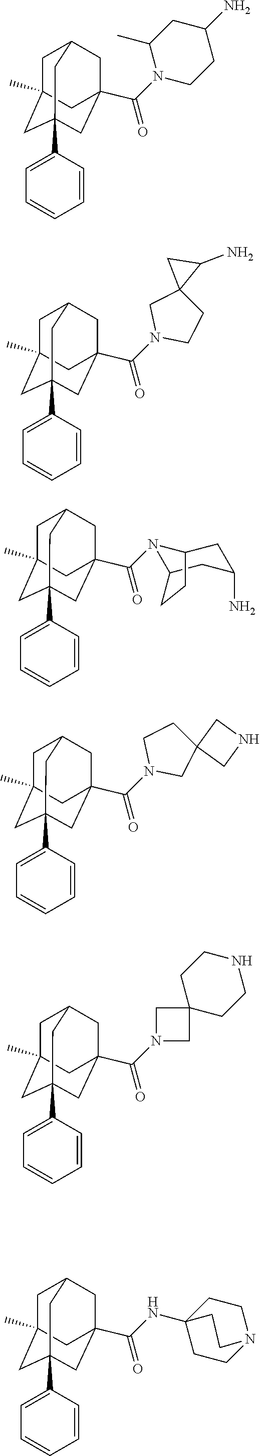

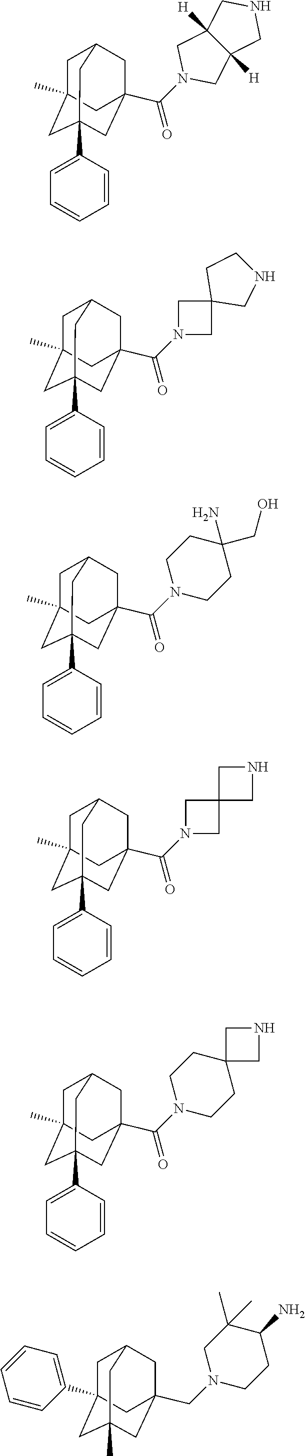 [SMILES: C[C@]12CC3CC(C(=O)N4C[C@H]5CNC[C@H]5C4)(C1)C[C@@](c1ccccc1)(C3)C2 ; C[C@]12CC3CC(C(=O)N4CC5(CCNC5)C4)(C1)C[C@@](c1ccccc1)(C3)C2 ; C[C@]12CC3CC(C(=O)N4CCC(N)(CO)CC4)(C1)C[C@@](c1ccccc1)(C3)C2 ; C[C@]12CC3CC(C(=O)N4CC5(CNC5)C4)(C1)C[C@@](c1ccccc1)(C3)C2 ; C[C@]12CC3CC(C(=O)N4CCC5(CC4)CNC5)(C1)C[C@@](c1ccccc1)(C3)C2 ; CC1(C)CN(CC23CC4C[C@](C)(C2)C[C@@](c2ccccc2)(C4)C3)CC[C@@H]1N]

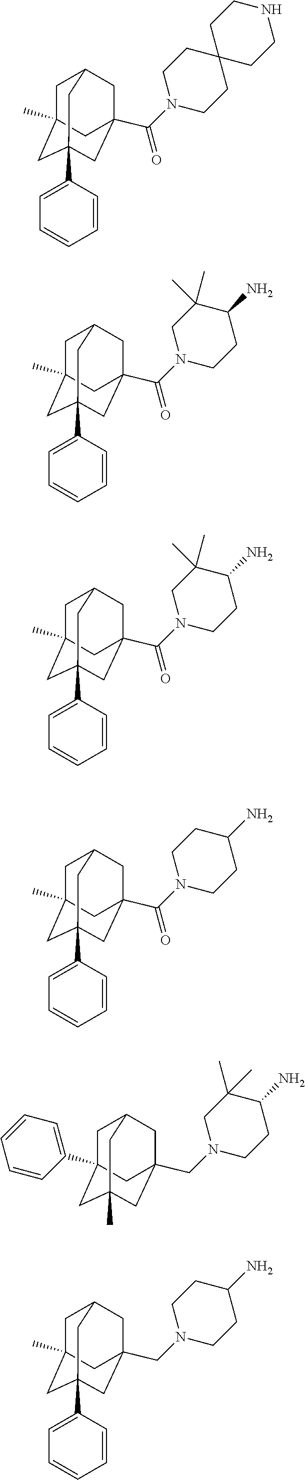 [SMILES: C[C@]12CC3CC(C(=O)N4CCC5(CCNCC5)CC4)(C1)C[C@@](c1ccccc1)(C3)C2 ; CC1(C)CN(C(=O)C23CC4C[C@@](C)(C2)C[C@](c2ccccc2)(C4)C3)CC[C@@H]1N ; CC1(C)CN(C(=O)C23CC4C[C@@](C)(C2)C[C@](c2ccccc2)(C4)C3)CC[C@H]1N ; C[C@]12CC3CC(C(=O)N4CCC(N)CC4)(C1)C[C@@](c1ccccc1)(C3)C2 ; CC1(C)CN(CC23CC4C[C@](C)(C2)C[C@@](c2ccccc2)(C4)C3)CC[C@H]1N ; C[C@]12CC3CC(CN4CCC(N)CC4)(C1)C[C@@](c1ccccc1)(C3)C2]

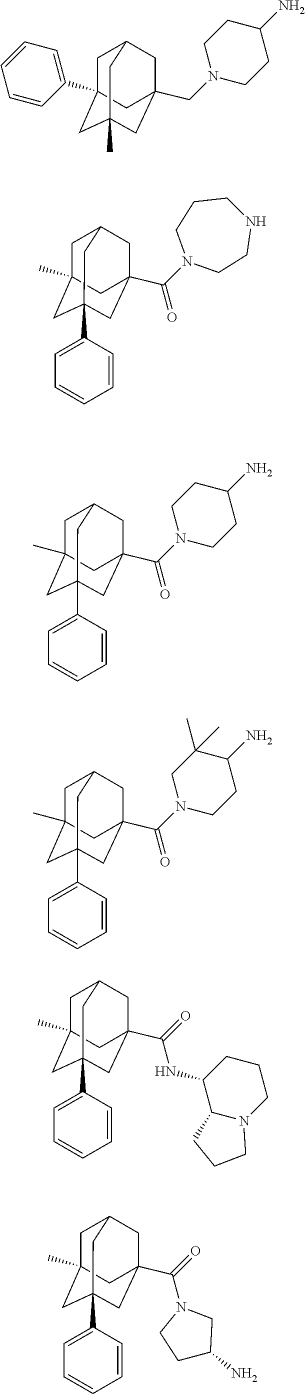

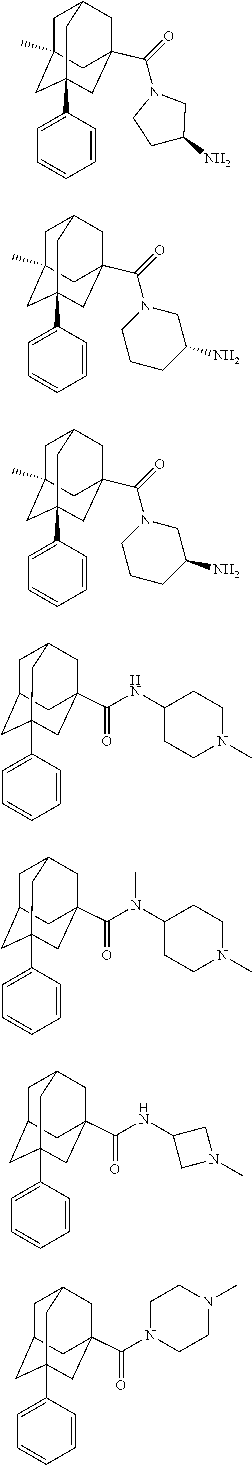

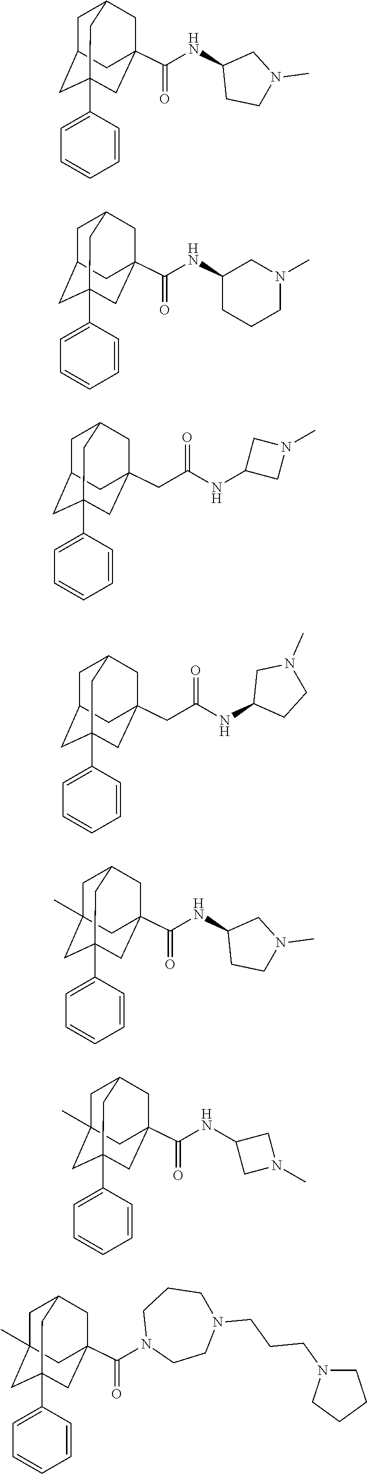

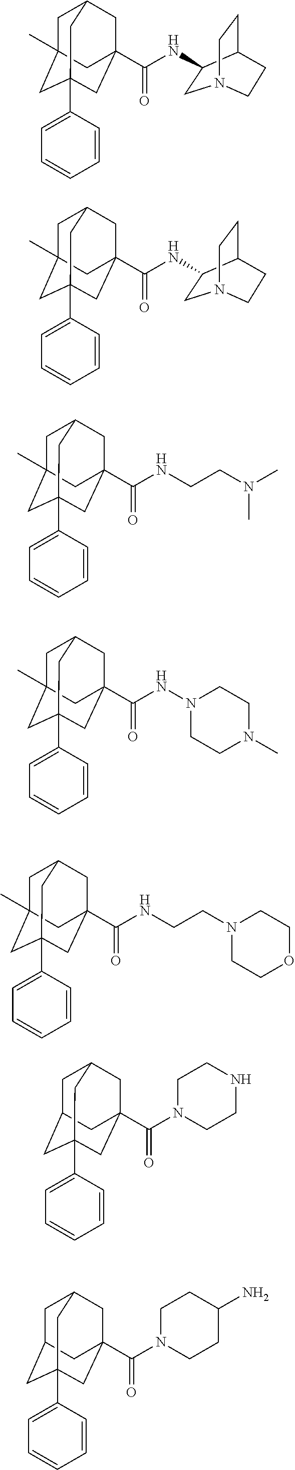

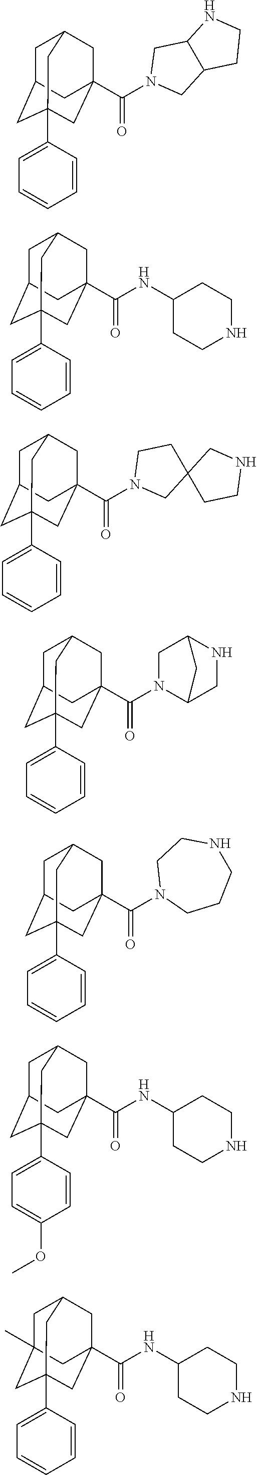

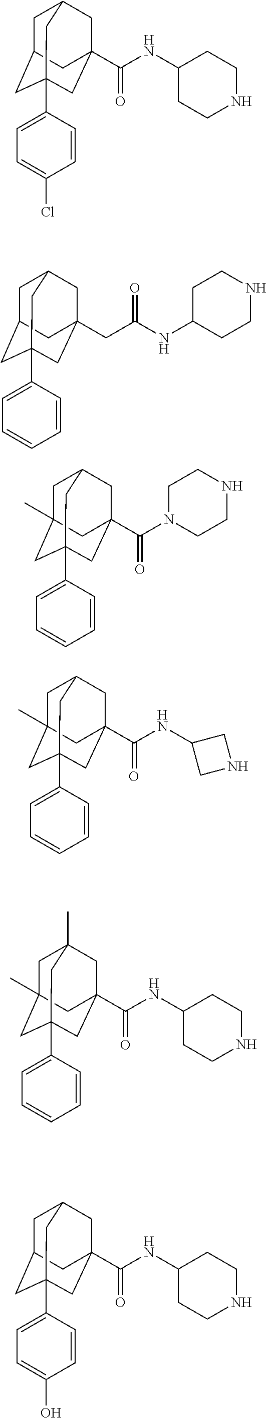

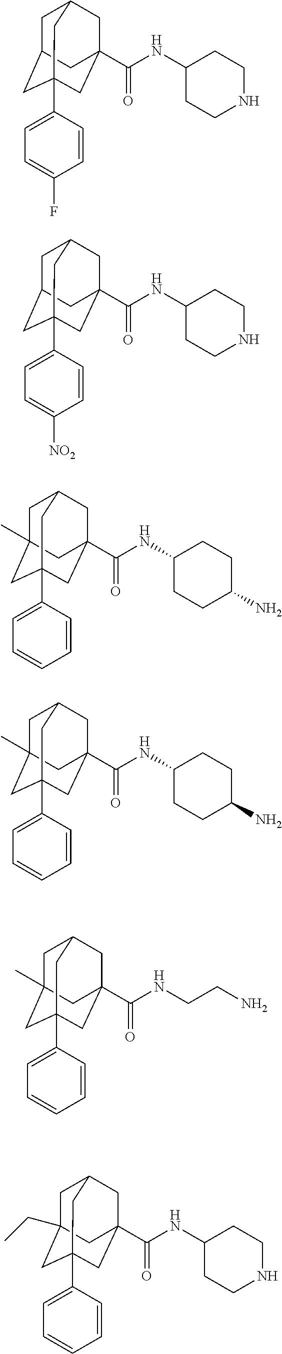

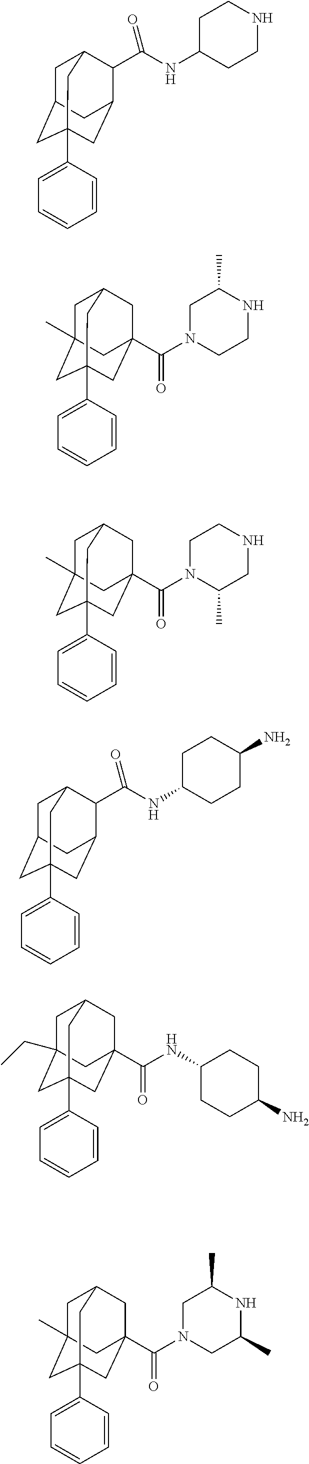

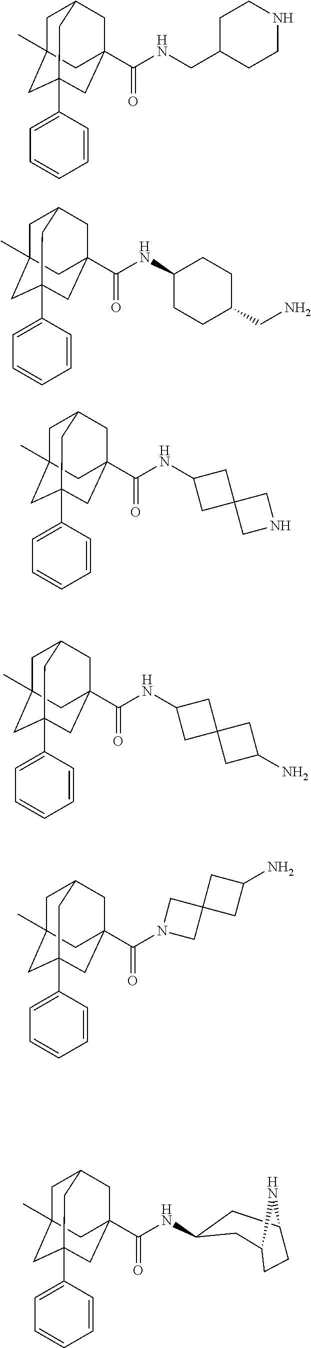

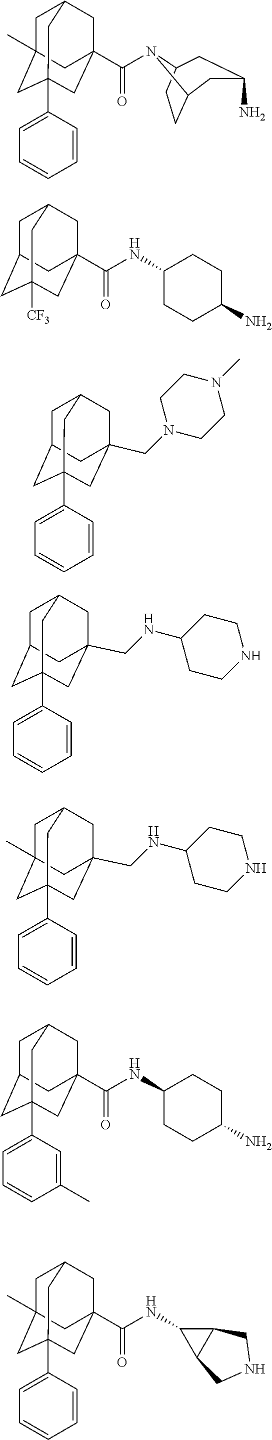

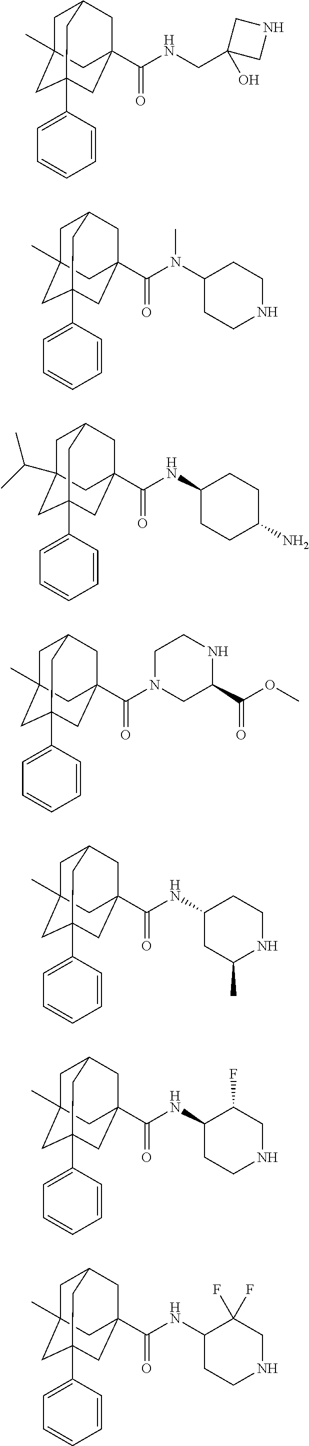

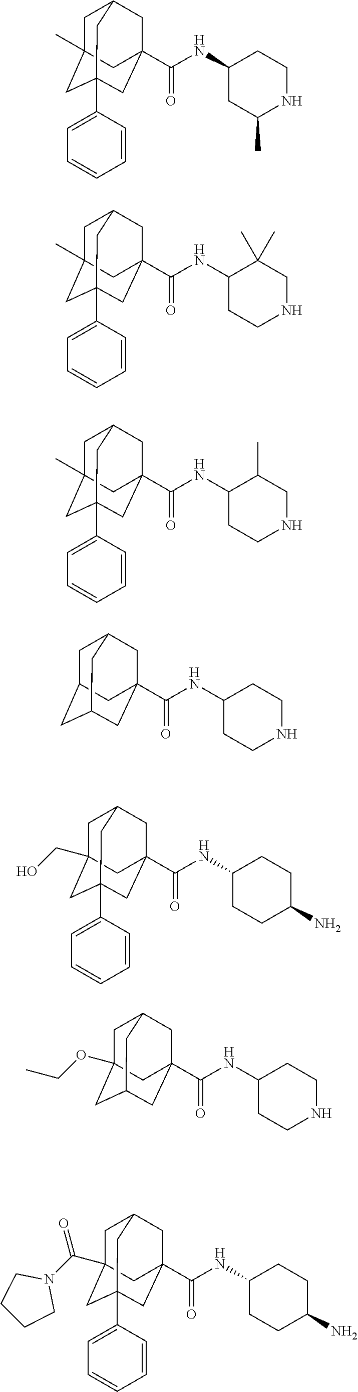

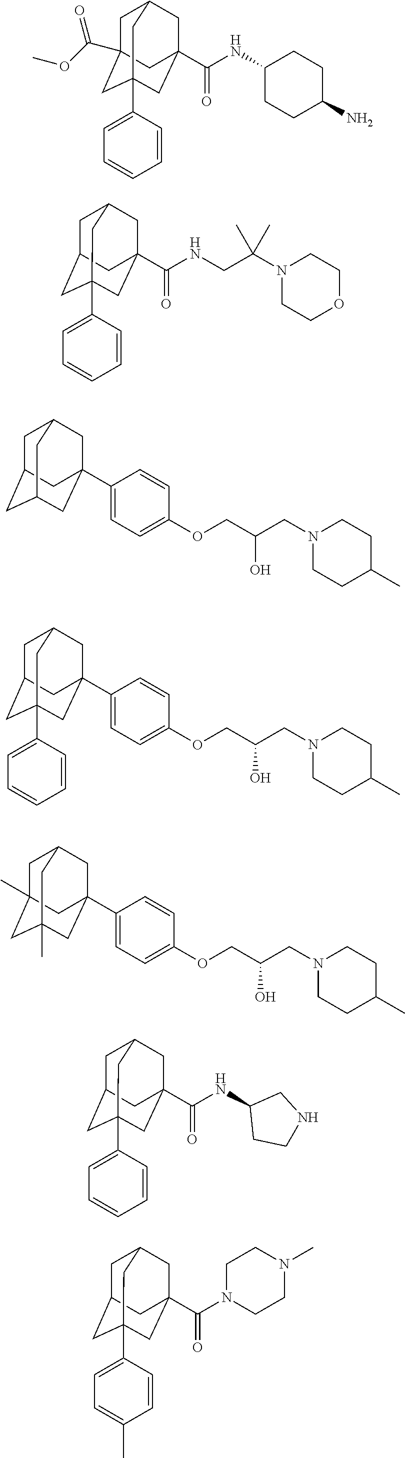

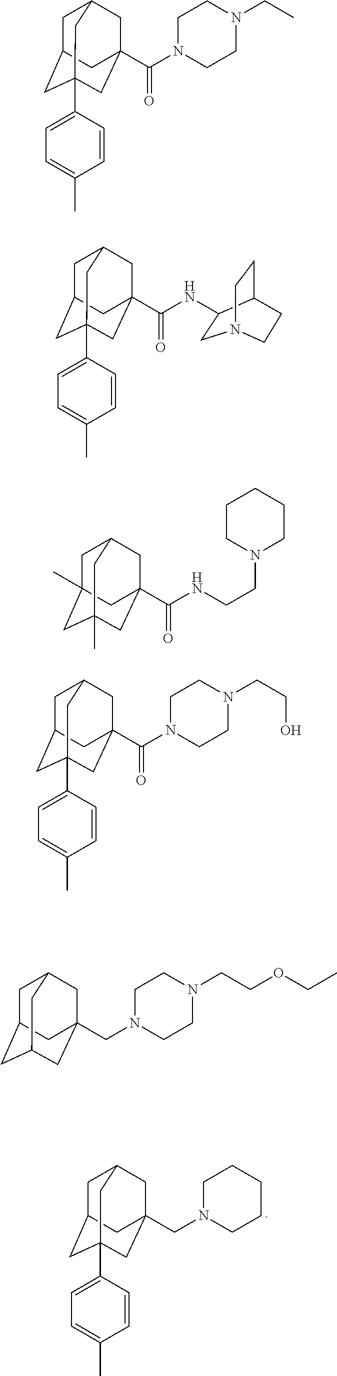

In another embodiment, the method comprises administering to humans, other mammals, cell culture, or biological sample a therapeutically effective amount of a compound represented by Structural Formula I for treatment of Ebolavirus infection,
or a pharmaceutically acceptable salt, and a pharmaceutically acceptable carrier, diluent, or vehicle thereof, wherein R¹, R², and NR^{3a}R^{3b} are defined as above and wherein
X is

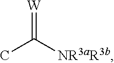

Y is CH₂, Q is CH₂ or CR²³R²⁴, and W is selected from O and S.

In another embodiment, the method comprises administering to humans, other mammals, cell culture, or biological sample a therapeutically effective amount of a compound represented by Structural Formula I for treatment of Ebolavirus infection,
or a pharmaceutically acceptable salt, and a pharmaceutically acceptable carrier, diluent, or vehicle thereof, wherein R¹, R², and NR^{3a}R^{3b} are defined as above and wherein
X is CH, Y is

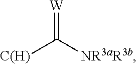

Q is CH₂, and W is selected from O and S.

In another embodiment, the method comprises administering to humans, other mammals, cell culture, or biological sample a therapeutically effective amount of an enantiomerically pure compound represented by Structural Formula II for treatment of Ebolavirus infection

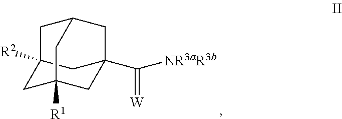

or a pharmaceutically acceptable salt, and a pharmaceutically acceptable carrier, diluent, or vehicle thereof, wherein R¹, R², NR^{3a}R^{3b} are defined as above, and W is selected from O and S.

In another embodiment, the method comprises administering to humans, other mammals, cell culture, or biological sample a therapeutically effective amount of an enantiomerically pure compound represented by Structural Formula III for treatment of Ebolavirus infection

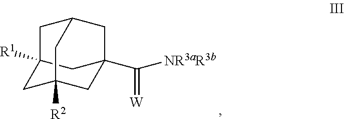

or a pharmaceutically acceptable salt, and a pharmaceutically acceptable carrier, diluent, or vehicle thereof, wherein R¹, R², NR^{3a}R^{3b} are defined as above, and W is selected from O and S.

In another embodiment, the method comprises administering to humans, other mammals, cell culture, or biological sample a therapeutically effective amount of a compound represented by Structural Formula IV for treatment of Ebolavirus infection

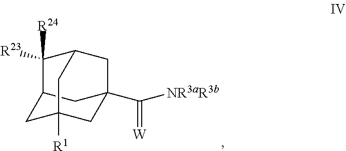

or a pharmaceutically acceptable salt, and a pharmaceutically acceptable carrier, diluent, or vehicle thereof, wherein R¹, R²³, R²⁴, NR^{3a}R^{3b} are defined as above, and W is selected from O and S.

In another embodiment, the method comprises administering to humans, other mammals, cell culture, or biological sample a therapeutically effective amount of a compound represented by Structural Formula V for treatment of Ebolavirus infection

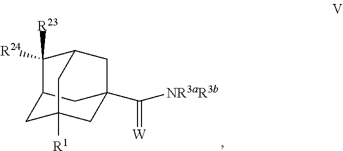

or a pharmaceutically acceptable salt, and a pharmaceutically acceptable carrier, diluent, or vehicle thereof, wherein R¹, R²³, R²⁴, NR^{3a}R^{3b} are defined as above, and W is selected from O and S.

In another embodiment, the method comprises administering to humans, other mammals, cell culture, or biological sample a therapeutically effective amount of a compound represented by Structural Formula VI for treatment of Ebolavirus infection

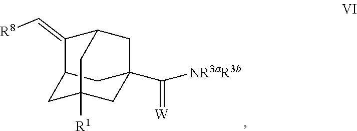

or a pharmaceutically acceptable salt, and a pharmaceutically acceptable carrier, diluent, or vehicle thereof, wherein R¹, R⁸, NR^{3a}R^{3b} are defined as above, and W is selected from O and S.

In another embodiment, the method comprises administering to humans, other mammals, cell culture, or biological sample a therapeutically effective amount of an enantiomerically pure compound represented by Structural Formula Via for treatment of Ebolavirus infection

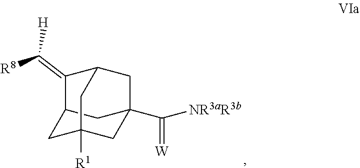

or a pharmaceutically acceptable salt, and a pharmaceutically acceptable carrier, diluent, or vehicle thereof, wherein R¹, R⁸, NR^{3a}R^{3b} are defined as above, and W is selected from O and S.

In another embodiment, the method comprises administering to humans, other mammals, cell culture, or biological sample a therapeutically effective amount of an enantiomerically pure compound represented by Structural Formula VIb for treatment of Ebolavirus infection

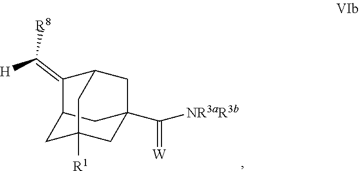

or a pharmaceutically acceptable salt, and a pharmaceutically acceptable carrier, diluent, or vehicle thereof, wherein R¹, R⁸, NR^{3a}R^{3b} are defined as above, and W is selected from O and S.

In another embodiment, the method comprises administering to humans, other mammals, cell culture, or biological sample a therapeutically effective amount of a compound represented by Structural Formula VII for treatment of Ebolavirus infection

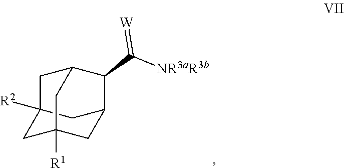

or a pharmaceutically acceptable salt, and a pharmaceutically acceptable carrier, diluent, or vehicle thereof, wherein R¹, R², NR^{3a}R^{3b} are defined as above, and W is selected from O and S.

In another embodiment, the method comprises administering to humans, other mammals, cell culture, or biological sample a therapeutically effective amount of a compound represented by Structural Formula VIII for treatment of Ebolavirus infection

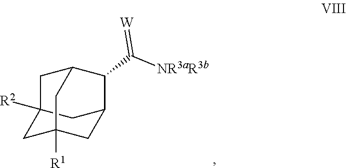

or a pharmaceutically acceptable salt, and a pharmaceutically acceptable carrier, diluent, or vehicle thereof, wherein R¹, R², NR^{3a}R^{3b} are defined as above, and W is selected from O and S.

In another embodiment, the method comprises administering to humans, other mammals, cell culture, or biological sample a therapeutically effective amount of a compound represented by Structural Formula I for treatment of Ebolavirus infection,
or a pharmaceutically acceptable salt, and a pharmaceutically acceptable carrier, diluent, or vehicle thereof, wherein R¹ and NR^{3a}R^{3b} are defined as above and wherein
X is

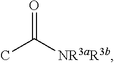

Y is CH₂, and Q is CH₂; and
R² is selected from the group consisting of

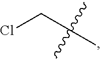

### Br,

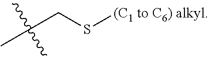

In another embodiment, the method comprises administering to humans, other mammals, cell culture, or biological sample a therapeutically effective amount of a compound represented by Structural Formula I for treatment of Ebolavirus infection,
or a pharmaceutically acceptable salt, and a pharmaceutically acceptable carrier, diluent, or vehicle thereof, wherein R¹, R², and NR^{3a}R^{3b} are defined as above and wherein
X is

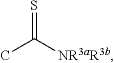

Y is CH₂, and Q is CH₂.

In another embodiment, the method comprises administering to humans, other mammals, cell culture, or biological sample a therapeutically effective amount of a compound represented by Structural Formula I for treatment of Ebolavirus infection,
or a pharmaceutically acceptable salt, and a pharmaceutically acceptable carrier, diluent, or vehicle thereof, wherein R¹ and NR^{3a}R^{3b} are defined as above and wherein
X is CH, Y is

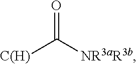

### and Q is CH₂; and

R² is selected from the group consisting of halogen, methyl, ethyl, propyl, chloromethyl, chloroethyl, methylthiomethyl, and thiomethyl.

In another embodiment, the method comprises administering to humans, other mammals, cell culture, or biological sample a therapeutically effective amount of a compound represented by Structural Formula I for treatment of Ebolavirus infection,
or a pharmaceutically acceptable salt, and a pharmaceutically acceptable carrier, diluent, or vehicle thereof, wherein R¹ and NR^{3a}R^{3b} are defined as above and wherein
X is CH, Y is

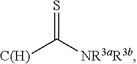

### and Q is CH₂; and

R² is selected from the group consisting of hydrogen, halogen, methyl, ethyl, propyl, chloromethyl, chloroethyl, methylthiomethyl, and thiomethyl.

In another embodiment, the method comprises administering to humans, other mammals, cell culture, or biological sample a therapeutically effective amount of a compound represented by Structural Formula I for treatment of Ebolavirus infection,
or a pharmaceutically acceptable salt, and a pharmaceutically acceptable carrier, diluent, or vehicle thereof, wherein R¹, R², R²³, R²⁴, and NR^{3a}R^{3b} are defined as above and wherein
X is

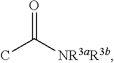

Y is CH₂, and Q is CR²³R²⁴.

In another embodiment, the method comprises administering to humans, other mammals, cell culture, or biological sample a therapeutically effective amount of a compound represented by Structural Formula I for treatment of Ebolavirus infection,
or a pharmaceutically acceptable salt, and a pharmaceutically acceptable carrier, diluent, or vehicle thereof, wherein R¹, R², R²³, R²⁴, and NR^{3a}R^{3b} are defined as above and wherein
X is

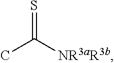

Y is CH₂, and Q is CR²³R²⁴

In another embodiment, the method comprises administering to humans, other mammals, cell culture, or biological sample a therapeutically effective amount of an enantiomerically pure compound represented by Structural Formula II for treatment of Ebolavirus infection,
or a pharmaceutically acceptable salt, and a pharmaceutically acceptable carrier, diluent, or vehicle thereof, wherein W is O, R¹ and NR^{3a}R^{3b} are defined as above and wherein
R² is selected from the group consisting of

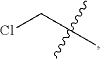

### Br,

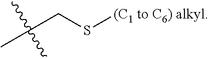

In another embodiment, the method comprises administering to humans, other mammals, cell culture, or biological sample a therapeutically effective amount of an enantiomerically pure compound represented by Structural Formula II for treatment of Ebolavirus infection,
or a pharmaceutically acceptable salt, and a pharmaceutically acceptable carrier, diluent, or vehicle thereof, wherein W is S and R¹, R², and NR^{3a}R^{3b} are defined as above.

In another embodiment, the method comprises administering to humans, other mammals, cell culture, or biological sample a therapeutically effective amount of an enantiomerically pure compound represented by Structural Formula III for treatment of Ebolavirus infection,
or a pharmaceutically acceptable salt, and a pharmaceutically acceptable carrier, diluent, or vehicle thereof, wherein W is O, R¹ and NR^{3a}R^{3b} are defined as above and wherein
R² is selected from the group consisting of

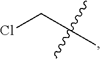

### Br,

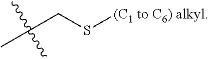

In another embodiment, the method comprises administering to humans, other mammals, cell culture, or biological sample a therapeutically effective amount of an enantiomerically pure compound represented by Structural Formula III for treatment of Ebolavirus infection,
or a pharmaceutically acceptable salt, and a pharmaceutically acceptable carrier, diluent, or vehicle thereof, wherein W is S and R¹, R², and NR^{3a}R^{3b} are defined as above.

In another embodiment, the method comprises administering to humans, other mammals, cell culture, or biological sample a therapeutically effective amount of a compound represented by Structural Formula IV for treatment of Ebolavirus infection,
or a pharmaceutically acceptable salt, and a pharmaceutically acceptable carrier, diluent, or vehicle thereof, wherein W is O and R¹, R²³, R²⁴, and NR^{3a}R^{3b} are defined as above.

In another embodiment, the method comprises administering to humans, other mammals, cell culture, or biological sample a therapeutically effective amount of a compound represented by Structural Formula IV for treatment of Ebolavirus infection,
or a pharmaceutically acceptable salt, and a pharmaceutically acceptable carrier, diluent, or vehicle thereof, wherein W is S and R¹, R²³, R²⁴, and NR^{3a}R^{3b} are defined as above.

In another embodiment, the method comprises administering to humans, other mammals, cell culture, or biological sample a therapeutically effective amount of a compound represented by Structural Formula V for treatment of Ebolavirus infection,
or a pharmaceutically acceptable salt, and a pharmaceutically acceptable carrier, diluent, or vehicle thereof, wherein W is O and R¹, R²³, R²⁴, and NR^{3a}R^{3b} are defined as above.

In another embodiment, the method comprises administering to humans, other mammals, cell culture, or biological sample a therapeutically effective amount of a compound represented by Structural Formula V for treatment of Ebolavirus infection,
or a pharmaceutically acceptable salt, and a pharmaceutically acceptable carrier, diluent, or vehicle thereof, wherein W is S and R¹, R²³, R²⁴, and NR^{3a}R^{3b} are defined as above.

In another embodiment, the method comprises administering to humans, other mammals, cell culture, or biological sample a therapeutically effective amount of a compound represented by Structural Formula VI for treatment of Ebolavirus infection,
or a pharmaceutically acceptable salt, and a pharmaceutically acceptable carrier, diluent, or vehicle thereof, wherein W is O and R¹, R⁸, and NR^{3a}R^{3b} are defined as above.

In another embodiment, the method comprises administering to humans, other mammals, cell culture, or biological sample a therapeutically effective amount of a compound represented by Structural Formula VI for treatment of Ebolavirus infection,
or a pharmaceutically acceptable salt, and a pharmaceutically acceptable carrier, diluent, or vehicle thereof, wherein W is S and R¹, R⁸, and NR^{3a}R^{3b} are defined as above.

In another embodiment, the method comprises administering to humans, other mammals, cell culture, or biological sample a therapeutically effective amount of an enantiomerically pure compound represented by Structural Formula Via for treatment of Ebolavirus infection,
or a pharmaceutically acceptable salt, and a pharmaceutically acceptable carrier, diluent, or vehicle thereof, wherein W is O and R¹, R⁸, and NR^{3a}R^{3b} are defined as above.

In another embodiment, the method comprises administering to humans, other mammals, cell culture, or biological sample a therapeutically effective amount of an enantiomerically pure compound represented by Structural Formula Via for treatment of Ebolavirus infection,
or a pharmaceutically acceptable salt, and a pharmaceutically acceptable carrier, diluent, or vehicle thereof, wherein W is S and R¹, R⁸, and NR^{3a}R^{3b} are defined as above.

In another embodiment, the method comprises administering to humans, other mammals, cell culture, or biological sample a therapeutically effective amount of an enantiomerically pure compound represented by Structural Formula VIb for treatment of Ebolavirus infection,
or a pharmaceutically acceptable salt, and a pharmaceutically acceptable carrier, diluent, or vehicle thereof, wherein W is O and R¹, R⁸, and NR^{3a}R^{3b} are defined as above.

In another embodiment, the method comprises administering to humans, other mammals, cell culture, or biological sample a therapeutically effective amount of an enantiomerically pure compound represented by Structural Formula VIb for treatment of Ebolavirus infection,
or a pharmaceutically acceptable salt, and a pharmaceutically acceptable carrier, diluent, or vehicle thereof, wherein W is S and R¹, R⁸, and NR^{3a}R^{3b} are defined as above.

In another embodiment, the method comprises administering to humans, other mammals, cell culture, or biological sample a therapeutically effective amount of a compound represented by Structural Formula VII for treatment of Ebolavirus infection,
or a pharmaceutically acceptable salt, and a pharmaceutically acceptable carrier, diluent, or vehicle thereof, wherein W is 0 and R¹ and NR^{3a}R^{3b} are defined as above and wherein
R² is selected from the group consisting of halogen, methyl, ethyl, propyl, chloromethyl, chloroethyl, methylthiomethyl, and thiomethyl.

In another embodiment, the method comprises administering to humans, other mammals, cell culture, or biological sample a therapeutically effective amount of a compound represented by Structural Formula VII for treatment of Ebolavirus infection,
or a pharmaceutically acceptable salt, and a pharmaceutically acceptable carrier, diluent, or vehicle thereof, wherein W is S and R¹ and NR^{3a}R^{3b} are defined as above and wherein
R² is selected from the group consisting of halogen, methyl, ethyl, propyl, chloromethyl, chloroethyl, methylthiomethyl, and thiomethyl.

In another embodiment, the method comprises administering to humans, other mammals, cell culture, or biological sample a therapeutically effective amount of a compound represented by Structural Formula VIII for treatment of Ebolavirus infection,
or a pharmaceutically acceptable salt, and a pharmaceutically acceptable carrier, diluent, or vehicle thereof, wherein W is O and R¹ and NR^{3a}R^{3b} are defined as above and wherein
R² is selected from the group consisting of halogen, methyl, ethyl, propyl, chloromethyl, chloroethyl, methylthiomethyl, and thiomethyl.

In another embodiment, the method comprises administering to humans, other mammals, cell culture, or biological sample a therapeutically effective amount of a compound represented by Structural Formula VIII for treatment of Ebolavirus infection,
or a pharmaceutically acceptable salt, and a pharmaceutically acceptable carrier, diluent, or vehicle thereof, wherein W is S and R¹ and NR^{3a}R^{3b} are defined as above and wherein
R² is selected from the group consisting of halogen, methyl, ethyl, propyl, chloromethyl, chloroethyl, methylthiomethyl, and thiomethyl.

In another embodiment, the method comprises administering to humans, other mammals, cell culture, or biological sample a therapeutically effective amount of a compound represented by Structural Formula I for treatment of Ebolavirus infection,
or a pharmaceutically acceptable salt, and a pharmaceutically acceptable carrier, diluent, or vehicle thereof, wherein NR^{3a}R^{3b} is defined as above and wherein
x is

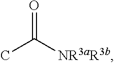

Y is CH₂, and Q is CH₂;
R¹ is phenyl; and
R² is selected from the group consisting of

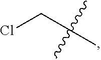

### Br,

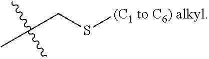

In another embodiment, the method comprises administering to humans, other mammals, cell culture, or biological sample a therapeutically effective amount of a compound represented by Structural Formula I for treatment of Ebolavirus infection,
or a pharmaceutically acceptable salt, and a pharmaceutically acceptable carrier, diluent, or vehicle thereof, wherein R² and NR^{3a}R^{3b} are defined as above and wherein
X is

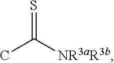

Y is CH₂, and Q is CH₂; and
R¹ is phenyl.

In another embodiment, the method comprises administering to humans, other mammals, cell culture, or biological sample a therapeutically effective amount of a compound represented by Structural Formula I for treatment of Ebolavirus infection,
or a pharmaceutically acceptable salt, and a pharmaceutically acceptable carrier, diluent, or vehicle thereof, wherein NR^{3a}R^{3b} is defined as above and wherein
X is CH, Y is

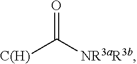

### and Q is CH₂;

R¹ is phenyl; and
R² is selected from the group consisting of halogen, methyl, ethyl, propyl, chloromethyl, chloroethyl, methylthiomethyl, and thiomethyl.

In another embodiment, the method comprises administering to humans, other mammals, cell culture, or biological sample a therapeutically effective amount of a compound represented by Structural Formula I for treatment of Ebolavirus infection,
or a pharmaceutically acceptable salt, and a pharmaceutically acceptable carrier, diluent, or vehicle thereof, wherein NR^{3a}R^{3b} is defined as above and wherein
X is CH, Y is

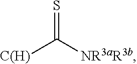

### and Q is CH₂;

R¹ is phenyl; and
R² is selected from the group consisting of hydrogen, halogen, methyl, ethyl, propyl, chloromethyl, chloroethyl, methylthiomethyl, and thiomethyl.

In another embodiment, the method comprises administering to humans, other mammals, cell culture, or biological sample a therapeutically effective amount of a compound represented by Structural Formula I for treatment of Ebolavirus infection,
or a pharmaceutically acceptable salt, and a pharmaceutically acceptable carrier, diluent, or vehicle thereof, wherein R², R²³, R²⁴, and NR^{3a}R^{3b} are defined as above and wherein
X is

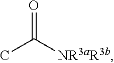

Y is CH₂, and Q is CR²³R²⁴; and
R¹ is phenyl.

In another embodiment, the method comprises administering to humans, other mammals, cell culture, or biological sample a therapeutically effective amount of a compound represented by Structural Formula I for treatment of Ebolavirus infection,
or a pharmaceutically acceptable salt, and a pharmaceutically acceptable carrier, diluent, or vehicle thereof, wherein R², R²³, R²⁴, and NR^{3a}R^{3b} are defined as above and wherein
X is

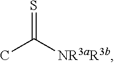

Y is CH₂, and Q is CR²³R²⁴; and
R¹ is phenyl.

In another embodiment, the method comprises administering to humans, other mammals, cell culture, or biological sample a therapeutically effective amount of an enantiomerically pure compound represented by Structural Formula II for treatment of Ebolavirus infection,
or a pharmaceutically acceptable salt, and a pharmaceutically acceptable carrier, diluent, or vehicle thereof, wherein W is O, R¹ is phenyl, NR^{3a}R^{3b} is defined as above and wherein
R² is selected from the group consisting of

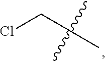 [SMILES: *CCl]

### Br,

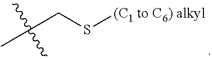

In another embodiment, the method comprises administering to humans, other mammals, cell culture, or biological sample a therapeutically effective amount of an enantiomerically pure compound represented by Structural Formula II for treatment of Ebolavirus infection,
or a pharmaceutically acceptable salt, and a pharmaceutically acceptable carrier, diluent, or vehicle thereof, wherein W is S, R¹ is phenyl, and R² and NR^{3a}R^{3b} are defined as above.

In another embodiment, the method comprises administering to humans, other mammals, cell culture, or biological sample a therapeutically effective amount of an enantiomerically pure compound represented by Structural Formula III for treatment of Ebolavirus infection,
or a pharmaceutically acceptable salt, and a pharmaceutically acceptable carrier, diluent, or vehicle thereof, wherein W is O, R¹ is phenyl, NR^{3a}R^{3b} is defined as above and wherein
R² is selected from the group consisting of

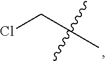 [SMILES: *CCl]

### Br, and

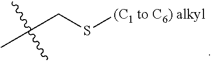

In another embodiment, the method comprises administering to humans, other mammals, cell culture, or biological sample a therapeutically effective amount of an enantiomerically pure compound represented by Structural Formula III for treatment of Ebolavirus infection,
or a pharmaceutically acceptable salt, and a pharmaceutically acceptable carrier, diluent, or vehicle thereof, wherein W is S, R¹ is phenyl, and R² and NR^{3a}R^{3b} are defined as above.

In another embodiment, the method comprises administering to humans, other mammals, cell culture, or biological sample a therapeutically effective amount of a compound represented by Structural Formula IV for treatment of Ebolavirus infection,
or a pharmaceutically acceptable salt, and a pharmaceutically acceptable carrier, diluent, or vehicle thereof, wherein W is O, R¹ is phenyl, and R²³, R²⁴, and NR^{3a}R^{3b} are defined as above.

In another embodiment, the method comprises administering to humans, other mammals, cell culture, or biological sample a therapeutically effective amount of a compound represented by Structural Formula IV for treatment of Ebolavirus infection,
or a pharmaceutically acceptable salt, and a pharmaceutically acceptable carrier, diluent, or vehicle thereof, wherein W is S, R¹ is phenyl, and R²³, R²⁴, and NR^{3a}R^{3b} are defined as above.

In another embodiment, the method comprises administering to humans, other mammals, cell culture, or biological sample a therapeutically effective amount of a compound represented by Structural Formula V for treatment of Ebolavirus infection,
or a pharmaceutically acceptable salt, and a pharmaceutically acceptable carrier, diluent, or vehicle thereof, wherein W is O, R¹ is phenyl, and R²³, R²⁴, and NR^{3a}R^{3b} are defined as above.

In another embodiment, the method comprises administering to humans, other mammals, cell culture, or biological sample a therapeutically effective amount of a compound represented by Structural Formula V for treatment of Ebolavirus infection,
or a pharmaceutically acceptable salt, and a pharmaceutically acceptable carrier, diluent, or vehicle thereof, wherein W is S, R¹ is phenyl, and R²³, R²⁴, and NR^{3a}R^{3b} are defined as above.

In another embodiment, the method comprises administering to humans, other mammals, cell culture, or biological sample a therapeutically effective amount of a compound represented by Structural Formula VI for treatment of Ebolavirus infection,
or a pharmaceutically acceptable salt, and a pharmaceutically acceptable carrier, diluent, or vehicle thereof, wherein W is O, R¹ is phenyl, and R⁸ and NR^{3a}R^{3b} are defined as above.

In another embodiment, the method comprises administering to humans, other mammals, cell culture, or biological sample a therapeutically effective amount of a compound represented by Structural Formula VI for treatment of Ebolavirus infection,
or a pharmaceutically acceptable salt, and a pharmaceutically acceptable carrier, diluent, or vehicle thereof, wherein W is O, R¹ is phenyl, NR^{3a}R^{3b} is defined as above and wherein
R⁸ is selected from the group consisting of methyl, ethyl, and propyl.

In another embodiment, the method comprises administering to humans, other mammals, cell culture, or biological sample a therapeutically effective amount of a compound represented by Structural Formula VI for treatment of Ebolavirus infection,
or a pharmaceutically acceptable salt, and a pharmaceutically acceptable carrier, diluent, or vehicle thereof, wherein W is S, R¹ is phenyl, and R⁸ and NR^{3a}R^{3b} are defined as above.

In another embodiment, the method comprises administering to humans, other mammals, cell culture, or biological sample a therapeutically effective amount of a compound represented by Structural Formula VI for treatment of Ebolavirus infection,
or a pharmaceutically acceptable salt, and a pharmaceutically acceptable carrier, diluent, or vehicle thereof, wherein W is S, R¹ is phenyl, NR^{3a}R^{3b} is defined as above and wherein
R⁸ is selected from the group consisting of methyl, ethyl, and propyl.

In another embodiment, the method comprises administering to humans, other mammals, cell culture, or biological sample a therapeutically effective amount of an enantiomerically pure compound represented by Structural Formula Via for treatment of Ebolavirus infection,
or a pharmaceutically acceptable salt, and a pharmaceutically acceptable carrier, diluent, or vehicle thereof, wherein W is O, R¹ is phenyl, and R⁸ and NR^{3a}R^{3b} are defined as above.

In another embodiment, the method comprises administering to humans, other mammals, cell culture, or biological sample a therapeutically effective amount of an enantiomerically pure compound represented by Structural Formula Via for treatment of Ebolavirus infection,
or a pharmaceutically acceptable salt, and a pharmaceutically acceptable carrier, diluent, or vehicle thereof, wherein W is O, R¹ is phenyl, NR^{3a}R^{3b} is defined as above and wherein
R⁸ is selected from the group consisting of methyl, ethyl, and propyl.

In another embodiment, the method comprises administering to humans, other mammals, cell culture, or biological sample a therapeutically effective amount of an enantiomerically pure compound represented by Structural Formula Via for treatment of Ebolavirus infection,
or a pharmaceutically acceptable salt, and a pharmaceutically acceptable carrier, diluent, or vehicle thereof, wherein W is S, R¹ is phenyl, and R⁸ and NR^{3a}R^{3b} are defined as above.

In another embodiment, the method comprises administering to humans, other mammals, cell culture, or biological sample a therapeutically effective amount of an enantiomerically pure compound represented by Structural Formula Via for treatment of Ebolavirus infection,
or a pharmaceutically acceptable salt, and a pharmaceutically acceptable carrier, diluent, or vehicle thereof, wherein W is S, R¹ is phenyl, NR^{3a}R^{3b} is defined as above and wherein
R⁸ is selected from the group consisting of methyl, ethyl, and propyl.

In another embodiment, the method comprises administering to humans, other mammals, cell culture, or biological sample a therapeutically effective amount of an enantiomerically pure compound represented by Structural Formula VIb for treatment of Ebolavirus infection,
or a pharmaceutically acceptable salt, and a pharmaceutically acceptable carrier, diluent, or vehicle thereof, wherein W is O, R¹ is phenyl, and R⁸ and NR^{3a}R^{3b} are defined as above.

In another embodiment, the method comprises administering to humans, other mammals, cell culture, or biological sample a therapeutically effective amount of an enantiomerically pure compound represented by Structural Formula VIb for treatment of Ebolavirus infection,
or a pharmaceutically acceptable salt, and a pharmaceutically acceptable carrier, diluent, or vehicle thereof, wherein W is O, R¹ is phenyl, NR^{3a}R^{3b} is defined as above and wherein
R⁸ is selected from the group consisting of methyl, ethyl, and propyl.

In another embodiment, the method comprises administering to humans, other mammals, cell culture, or biological sample a therapeutically effective amount of an enantiomerically pure compound represented by Structural Formula VIb for treatment of Ebolavirus infection,
or a pharmaceutically acceptable salt, and a pharmaceutically acceptable carrier, diluent, or vehicle thereof, wherein W is S, R¹ is phenyl, and R⁸ and NR^{3a}R^{3b} are defined as above.

In another embodiment, the method comprises administering to humans, other mammals, cell culture, or biological sample a therapeutically effective amount of an enantiomerically pure compound represented by Structural Formula VIb for treatment of Ebolavirus infection,
or a pharmaceutically acceptable salt, and a pharmaceutically acceptable carrier, diluent, or vehicle thereof, wherein W is S, R¹ is phenyl, NR^{3a}R^{3b} is defined as above and wherein
R⁸ is selected from the group consisting of methyl, ethyl, and propyl.

In another embodiment, the method comprises administering to humans, other mammals, cell culture, or biological sample a therapeutically effective amount of a compound represented by Structural Formula VII for treatment of Ebolavirus infection,
or a pharmaceutically acceptable salt, and a pharmaceutically acceptable carrier, diluent, or vehicle thereof, wherein W is O, R¹ is phenyl, NR^{3a}R^{3b} is defined as above and wherein
R² is selected from the group consisting of halogen, methyl, ethyl, propyl, chloromethyl, chloroethyl, methylthiomethyl, and thiomethyl.

In another embodiment, the method comprises administering to humans, other mammals, cell culture, or biological sample a therapeutically effective amount of a compound represented by Structural Formula VII for treatment of Ebolavirus infection,
or a pharmaceutically acceptable salt, and a pharmaceutically acceptable carrier, diluent, or vehicle thereof, wherein W is S, R¹ is phenyl, NR^{3a}R^{3b} is defined as above and wherein
R² is selected from the group consisting of hydrogen, halogen, methyl, ethyl, propyl, chloromethyl, chloroethyl, methylthiomethyl, and thiomethyl.

In another embodiment, the method comprises administering to humans, other mammals, cell culture, or biological sample a therapeutically effective amount of a compound represented by Structural Formula VIII for treatment of Ebolavirus infection,
or a pharmaceutically acceptable salt, and a pharmaceutically acceptable carrier, diluent, or vehicle thereof, wherein W is O, R¹ is phenyl, NR^{3a}R^{3b} is defined as above and wherein
R² is selected from the group consisting of halogen, methyl, ethyl, propyl, chloromethyl, chloroethyl, methylthiomethyl, and thiomethyl.

In another embodiment, the method comprises administering to humans, other mammals, cell culture, or biological sample a therapeutically effective amount of a compound represented by Structural Formula VIII for treatment of Ebolavirus infection,
or a pharmaceutically acceptable salt, and a pharmaceutically acceptable carrier, diluent, or vehicle thereof, wherein W is S, R¹ is phenyl, NR^{3a}R^{3b} is defined as above and wherein
R² is selected from the group consisting of hydrogen, halogen, methyl, ethyl, propyl, chloromethyl, chloroethyl, methylthiomethyl, and thiomethyl.

In another embodiment, the method comprises administering to humans, other mammals, cell culture, or biological sample a therapeutically effective amount of a compound represented by Structural Formula I for treatment of Ebolavirus infection,
or a pharmaceutically acceptable salt, and a pharmaceutically acceptable carrier, diluent, or vehicle thereof, wherein R¹ is defined as above and wherein
X is

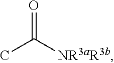

Y is CH₂, and Q is CH₂;
R² is selected from the group consisting of

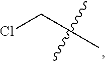 [SMILES: *CCl]

### Br,

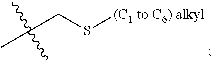

and
NR^{3a}R^{3b} is selected from the group consisting of the substituents of Table 5.

In another embodiment, the method comprises administering to humans, other mammals, cell culture, or biological sample a therapeutically effective amount of a compound represented by Structural Formula I for treatment of Ebolavirus infection,
or a pharmaceutically acceptable salt, and a pharmaceutically acceptable carrier, diluent, or vehicle thereof, wherein R¹, R², and NR^{3a}R^{3b} are defined as above and wherein
X is

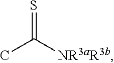

Y is CH₂, and Q is CH₂.

In another embodiment, the method comprises administering to humans, other mammals, cell culture, or biological sample a therapeutically effective amount of a compound represented by Structural Formula I for treatment of Ebolavirus infection,
or a pharmaceutically acceptable salt, and a pharmaceutically acceptable carrier, diluent, or vehicle thereof, wherein R¹ is defined as above and wherein
X is CH, Y is

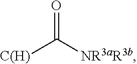

### and Q is CH₂;

R² is selected from the group consisting of halogen, methyl, ethyl, propyl, chloromethyl, chloroethyl, methylthiomethyl, and thiomethyl; and
NR^{3a}R^{3b} is selected from the group consisting of the substituents of Table 5.

In another embodiment, the method comprises administering to humans, other mammals, cell culture, or biological sample a therapeutically effective amount of a compound represented by Structural Formula I for treatment of Ebolavirus infection,
or a pharmaceutically acceptable salt, and a pharmaceutically acceptable carrier, diluent, or vehicle thereof, wherein R¹ is defined as above and wherein
X is CH, Y is

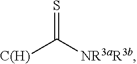

### and Q is CH₂;

R² is selected from the group consisting of hydrogen, halogen, methyl, ethyl, propyl, chloromethyl, chloroethyl, methylthiomethyl, and thiomethyl; and
NR^{3a}R^{3b} is selected from the group consisting of the substituents of Table 5.

In another embodiment, the method comprises administering to humans, other mammals, cell culture, or biological sample a therapeutically effective amount of a compound represented by Structural Formula I for treatment of Ebolavirus infection,
or a pharmaceutically acceptable salt, and a pharmaceutically acceptable carrier, diluent, or vehicle thereof, wherein R¹, R², R²³, and R²⁴ are defined as above and wherein
X is

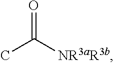

Y is CH₂, and Q is CR²³R²⁴; and
NR^{3a}R^{3b} is selected from the group consisting of the substituents of Table 5.

In another embodiment, the method comprises administering to humans, other mammals, cell culture, or biological sample a therapeutically effective amount of a compound represented by Structural Formula I for treatment of Ebolavirus infection,
or a pharmaceutically acceptable salt, and a pharmaceutically acceptable carrier, diluent, or vehicle thereof, wherein R¹, R², R²³, and R²⁴ are defined as above and wherein
X is

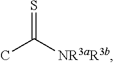

Y is CH₂, and Q is CR²³R²⁴; and
NR^{3a}R^{3b} is selected from the group consisting of the substituents of Table 5.

In another embodiment, the method comprises administering to humans, other mammals, cell culture, or biological sample a therapeutically effective amount of an enantiomerically pure compound represented by Structural Formula II for treatment of Ebolavirus infection,
or a pharmaceutically acceptable salt, and a pharmaceutically acceptable carrier, diluent, or vehicle thereof, wherein W is O, R¹ is defined as above and wherein
R² is selected from the group consisting of

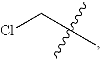

### Br,

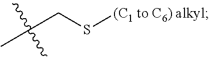

and
NR^{3a}R^{3b} is selected from the group consisting of the substituents of Table 5.

In another embodiment, the method comprises administering to humans, other mammals, cell culture, or biological sample a therapeutically effective amount of an enantiomerically pure compound represented by Structural Formula II for treatment of Ebolavirus infection,
or a pharmaceutically acceptable salt, and a pharmaceutically acceptable carrier, diluent, or vehicle thereof, wherein W is S and R¹, R², and NR^{3a}R^{3b} are defined as above.

In another embodiment, the method comprises administering to humans, other mammals, cell culture, or biological sample a therapeutically effective amount of an enantiomerically pure compound represented by Structural Formula III for treatment of Ebolavirus infection,
or a pharmaceutically acceptable salt, and a pharmaceutically acceptable carrier, diluent, or vehicle thereof, wherein W is O, R¹ is defined as above and wherein
R² is selected from the group consisting of

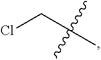

### Br,

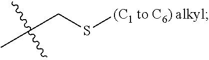

and
NR^{3a}R^{3b} is selected from the group consisting of the substituents of Table 5.

In another embodiment, the method comprises administering to humans, other mammals, cell culture, or biological sample a therapeutically effective amount of an enantiomerically pure compound represented by Structural Formula III for treatment of Ebolavirus infection,
or a pharmaceutically acceptable salt, and a pharmaceutically acceptable carrier, diluent, or vehicle thereof, wherein W is S and R¹, R², and NR^{3a}R^{3b} are defined as above.

In another embodiment, the method comprises administering to humans, other mammals, cell culture, or biological sample a therapeutically effective amount of a compound represented by Structural Formula IV for treatment of Ebolavirus infection,
or a pharmaceutically acceptable salt, and a pharmaceutically acceptable carrier, diluent, or vehicle thereof, wherein W is O and R¹, R²³, R²⁴, and NR^{3a}R^{3b} are defined as above.

In another embodiment, the method comprises administering to humans, other mammals, cell culture, or biological sample a therapeutically effective amount of a compound represented by Structural Formula IV for treatment of Ebolavirus infection,
or a pharmaceutically acceptable salt, and a pharmaceutically acceptable carrier, diluent, or vehicle thereof, wherein W is S and R¹, R²³, R²⁴, and NR^{3a}R^{3b} are defined as above.

In another embodiment, the method comprises administering to humans, other mammals, cell culture, or biological sample a therapeutically effective amount of a compound represented by Structural Formula V for treatment of Ebolavirus infection,
or a pharmaceutically acceptable salt, and a pharmaceutically acceptable carrier, diluent, or vehicle thereof, wherein W is O and R¹, R²³, R²⁴, and NR^{3a}R^{3b} are defined as above.

In another embodiment, the method comprises administering to humans, other mammals, cell culture, or biological sample a therapeutically effective amount of a compound represented by Structural Formula V for treatment of Ebolavirus infection,
or a pharmaceutically acceptable salt, and a pharmaceutically acceptable carrier, diluent, or vehicle thereof, wherein W is S and R¹, R²³, R²⁴, and NR^{3a}R^{3b} are defined as above.

In another embodiment, the method comprises administering to humans, other mammals, cell culture, or biological sample a therapeutically effective amount of a compound represented by Structural Formula VI for treatment of Ebolavirus infection,
or a pharmaceutically acceptable salt, and a pharmaceutically acceptable carrier, diluent, or vehicle thereof, wherein W is O and R¹, R⁸, and NR^{3a}R^{3b} are defined as above.

In another embodiment, the method comprises administering to humans, other mammals, cell culture, or biological sample a therapeutically effective amount of a compound represented by Structural Formula VI for treatment of Ebolavirus infection,
or a pharmaceutically acceptable salt, and a pharmaceutically acceptable carrier, diluent, or vehicle thereof, wherein W is S and R¹, R⁸, and NR^{3a}R^{3b} are defined as above.

In another embodiment, the method comprises administering to humans, other mammals, cell culture, or biological sample a therapeutically effective amount of an enantiomerically pure compound represented by Structural Formula Via for treatment of Ebolavirus infection,
or a pharmaceutically acceptable salt, and a pharmaceutically acceptable carrier, diluent, or vehicle thereof, wherein W is O and R¹, R⁸, and NR^{3a}R^{3b} are defined as above.

In another embodiment, the method comprises administering to humans, other mammals, cell culture, or biological sample a therapeutically effective amount of an enantiomerically pure compound represented by Structural Formula Via for treatment of Ebolavirus infection,
or a pharmaceutically acceptable salt, and a pharmaceutically acceptable carrier, diluent, or vehicle thereof, wherein W is S and R¹, R⁸, and NR^{3a}R^{3b} are defined as above.

In another embodiment, the method comprises administering to humans, other mammals, cell culture, or biological sample a therapeutically effective amount of an enantiomerically pure compound represented by Structural Formula VIb for treatment of Ebolavirus infection,
or a pharmaceutically acceptable salt, and a pharmaceutically acceptable carrier, diluent, or vehicle thereof, wherein W is O and R¹, R⁸, and NR^{3a}R^{3b} are defined as above.

In another embodiment, the method comprises administering to humans, other mammals, cell culture, or biological sample a therapeutically effective amount of an enantiomerically pure compound represented by Structural Formula VIb for treatment of Ebolavirus infection,
or a pharmaceutically acceptable salt, and a pharmaceutically acceptable carrier, diluent, or vehicle thereof, wherein W is S and R¹, R⁸, and NR^{3a}R^{3b} are defined as above.

In another embodiment, the method comprises administering to humans, other mammals, cell culture, or biological sample a therapeutically effective amount of a compound represented by Structural Formula VII for treatment of Ebolavirus infection,
or a pharmaceutically acceptable salt, and a pharmaceutically acceptable carrier, diluent, or vehicle thereof, wherein W is O, R¹ is defined above and wherein
R² is selected from the group consisting of halogen, methyl, ethyl, propyl, chloromethyl, chloroethyl, methylthiomethyl, and thiomethyl; and
NR^{3a}R^{3b} is selected from the group consisting of the substituents of Table 5.

In another embodiment, the method comprises administering to humans, other mammals, cell culture, or biological sample a therapeutically effective amount of a compound represented by Structural Formula VII for treatment of Ebolavirus infection,
or a pharmaceutically acceptable salt, and a pharmaceutically acceptable carrier, diluent, or vehicle thereof, wherein W is S, R¹ is defined above and wherein
R² is selected from the group consisting of hydrogen, halogen, methyl, ethyl, propyl, chloromethyl, chloroethyl, methylthiomethyl, and thiomethyl; and
NR^{3a}R^{3b} is selected from the group consisting of the substituents of Table 5.

In another embodiment, the method comprises administering to humans, other mammals, cell culture, or biological sample a therapeutically effective amount of a compound represented by Structural Formula VIII for treatment of Ebolavirus infection,
or a pharmaceutically acceptable salt, and a pharmaceutically acceptable carrier, diluent, or vehicle thereof, wherein W is O, R¹ is defined above and wherein
R² is selected from the group consisting of halogen, methyl, ethyl, propyl, chloromethyl, chloroethyl, methylthiomethyl, and thiomethyl; and
NR^{3a}R^{3b} is selected from the group consisting of the substituents of Table 5.

In another embodiment, the method comprises administering to humans, other mammals, cell culture, or biological sample a therapeutically effective amount of a compound represented by Structural Formula VIII for treatment of Ebolavirus infection,
or a pharmaceutically acceptable salt, and a pharmaceutically acceptable carrier, diluent, or vehicle thereof, wherein W is S, R¹ is defined above and wherein
R² is selected from the group consisting of hydrogen, halogen, methyl, ethyl, propyl, chloromethyl, chloroethyl, methylthiomethyl, and thiomethyl; and
NR^{3a}R^{3b} is selected from the group consisting of the substituents of Table 5.

In another embodiment, the method comprises administering to humans, other mammals, cell culture, or biological sample a therapeutically effective amount of an enantiomerically pure compound represented by Structural Formula II for treatment of Ebolavirus infection,
or a pharmaceutically acceptable salt, and a pharmaceutically acceptable carrier, diluent, or vehicle thereof, wherein W is S and R¹, R², and NR^{3a}R^{3b} are defined as above.

In another embodiment, the method comprises administering to humans, other mammals, cell culture, or biological sample a therapeutically effective amount of an enantiomerically pure compound represented by Structural Formula III for treatment of Ebolavirus infection,
or a pharmaceutically acceptable salt, and a pharmaceutically acceptable carrier, diluent, or vehicle thereof, wherein W is S and R¹, R², and NR^{3a}R^{3b} are defined as above.

In another embodiment, the method comprises administering to humans, other mammals, cell culture, or biological sample a therapeutically effective amount of a compound represented by Structural Formula IV for treatment of Ebolavirus infection,
or a pharmaceutically acceptable salt, and a pharmaceutically acceptable carrier, diluent, or vehicle thereof, wherein W is O or S, and R¹, R²³, R²⁴, and NR^{3a}R^{3b} are defined as above.

In another embodiment, the method comprises administering to humans, other mammals, cell culture, or biological sample a therapeutically effective amount of a compound represented by Structural Formula V for treatment of Ebolavirus infection,
or a pharmaceutically acceptable salt, and a pharmaceutically acceptable carrier, diluent, or vehicle thereof, wherein W is O or S, and R¹, R²³, R²⁴, and NR^{3a}R^{3b} are defined as above.

In another embodiment, the method comprises administering to humans, other mammals, cell culture, or biological sample a therapeutically effective amount of a compound represented by Structural Formula VI for treatment of Ebolavirus infection,
or a pharmaceutically acceptable salt, and a pharmaceutically acceptable carrier, diluent, or vehicle thereof, wherein W is O or S, and R¹, R⁸, and NR^{3a}R^{3b} are defined as above.

In another embodiment, the method comprises administering to humans, other mammals, cell culture, or biological sample a therapeutically effective amount of an enantiomerically pure compound represented by Structural Formula Via for treatment of Ebolavirus infection,
or a pharmaceutically acceptable salt, and a pharmaceutically acceptable carrier, diluent, or vehicle thereof, wherein W is O or S, and R¹, R⁸, and NR^{3a}R^{3b} are defined as above.

In another embodiment, the method comprises administering to humans, other mammals, cell culture, or biological sample a therapeutically effective amount of an enantiomerically pure compound represented by Structural Formula VIb for treatment of Ebolavirus infection,
or a pharmaceutically acceptable salt, and a pharmaceutically acceptable carrier, diluent, or vehicle thereof, wherein W is O or S, and R¹, R⁸, and NR^{3a}R^{3b} are defined as above.

In another embodiment, the method comprises administering to humans, other mammals, cell culture, or biological sample a therapeutically effective amount of a compound represented by Structural Formula VII for treatment of Ebolavirus infection,
or a pharmaceutically acceptable salt, and a pharmaceutically acceptable carrier, diluent, or vehicle thereof, wherein W is O or S, and R¹, R², and NR^{3a}R^{3b} are defined as above.

In another embodiment, the method comprises administering to humans, other mammals, cell culture, or biological sample a therapeutically effective amount of a compound represented by Structural Formula VIII for treatment of Ebolavirus infection,
or a pharmaceutically acceptable salt, and a pharmaceutically acceptable carrier, diluent, or vehicle thereof, wherein W is O or S, and R¹, R², and NR^{3a}R^{3b} are defined as above.

In another embodiment, the method comprises of including administering a therapeutic amount of a therapeutic agent selected from the group consisting of Ribavirin, viral RNA-dependent-RNA polymeras inhibitors including favipiravir, Triazavirin, Remdesivir (GS-5734), monoclonal antibody therapies including, ZMapp, REGN3470-3471-3479, mAb 114, vaccines including, cAd3-EBOZ, rVSV-ZEBOV, small interfering RNAs and microRNAs and immunomodulators.

In another embodiment, the method comprises the inhibiting of Ebolavirus glycoprotein.

In another embodiment, the method comprises administering to humans, other mammals, cell culture, or biological sample a therapeutically effective amount of compound selected from the group consisting of the compounds described as examples A1 to A7, B1 to B3, C1 to C3, D1, D2, E1 to E8, F1 to F5, G1 to G19, and H1 to H6 for treatment of Ebolavirus infection, or pharmaceutically acceptable salts, and a pharmaceutically acceptable carrier, diluent, or vehicle thereof, wherein R¹, R², X, Y, Q, W, and NR^{3a}R^{3b} are defined as above.

In one embodiment, the invention relates to compounds represented by Structural Formulae I, II, III, IV, V, VI, VIa, VIb, VII, and VIII

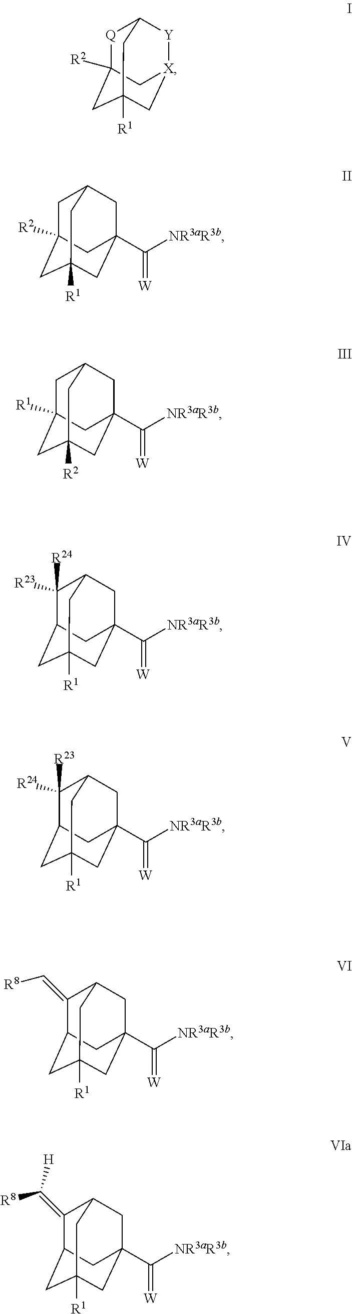

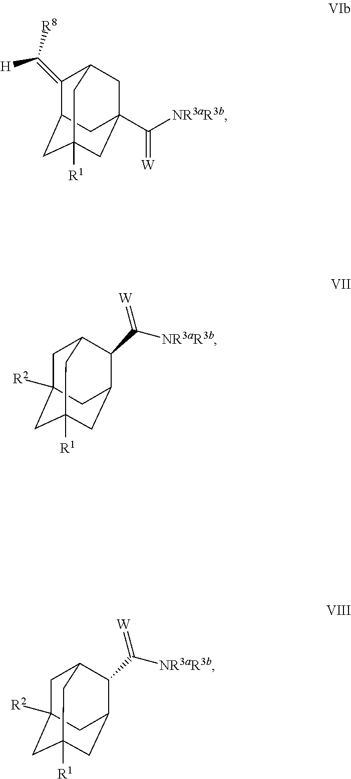

or a pharmaceutically acceptable salt, and a pharmaceutically acceptable carrier, diluent, or vehicle thereof described above.

In another embodiment, the invention relates to compounds, or pharmaceutically acceptable salts, and a pharmaceutically acceptable carrier, diluent, or vehicle thereof, selected from the group consisting of the compounds described as examples A1 to A7, B1 to B3, C1 to C3, D1, D2, E1 to E8, F1 to F5, G1 to G19, and H1 to H6.

In another embodiment, the invention can relate to compounds, or a pharmaceutically acceptable salt, and a pharmaceutically acceptable carrier, diluent, or vehicle thereof, selected from the group consisting of:

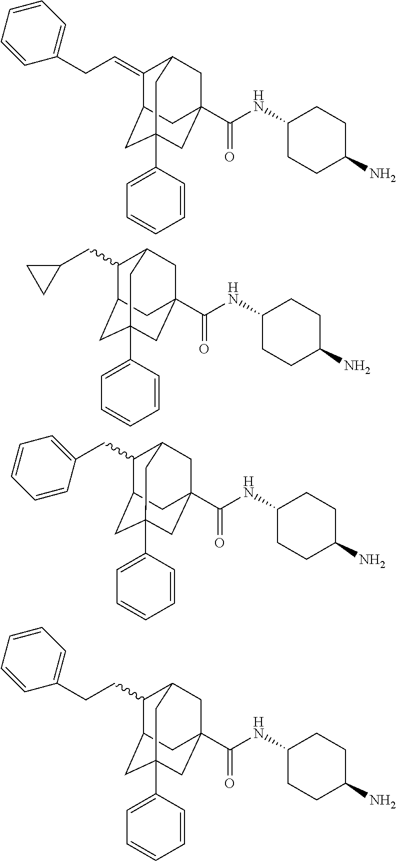

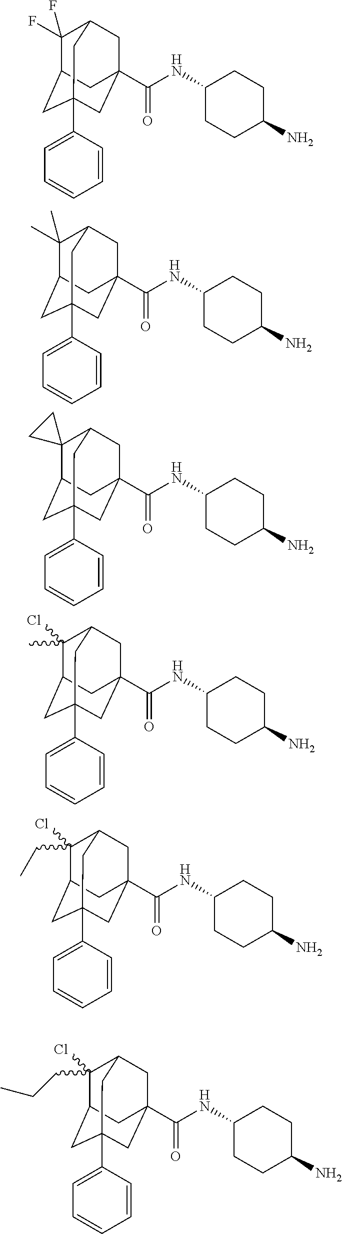

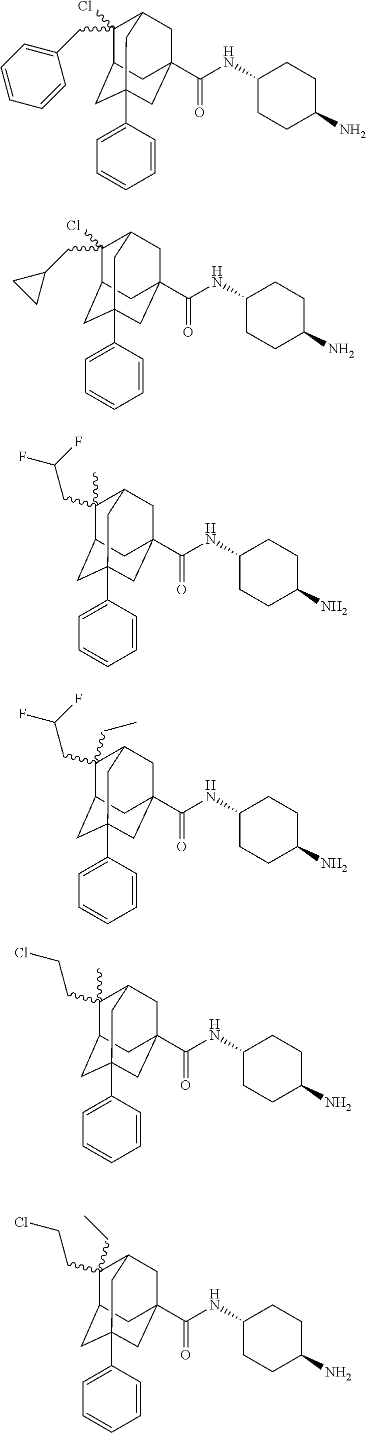

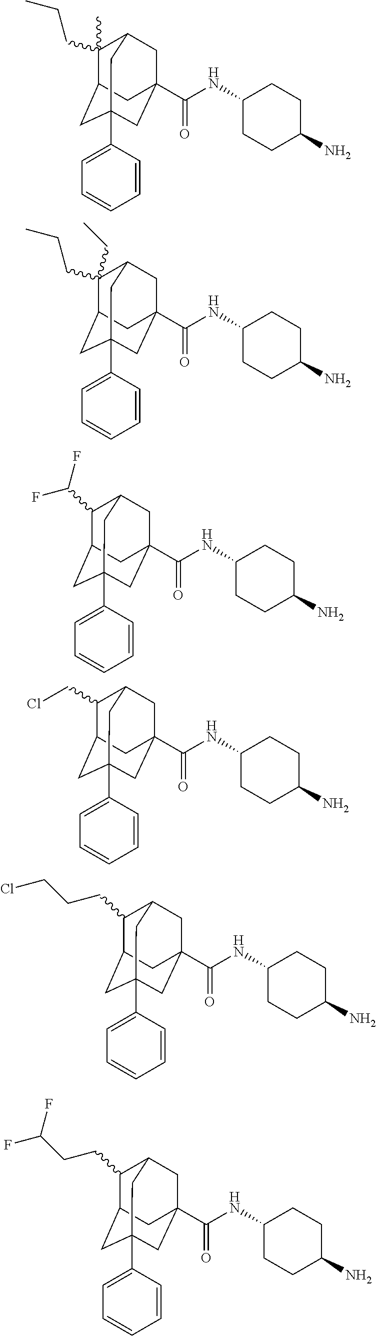

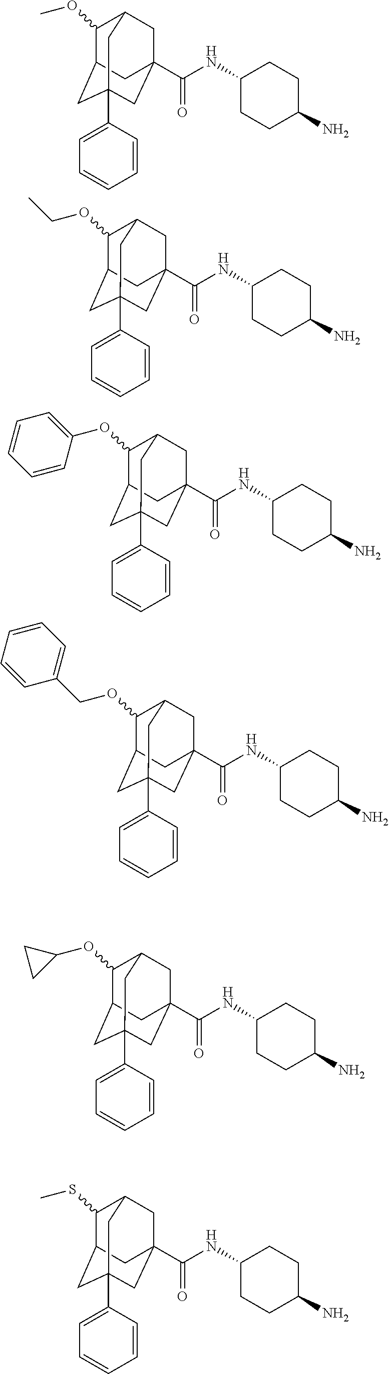

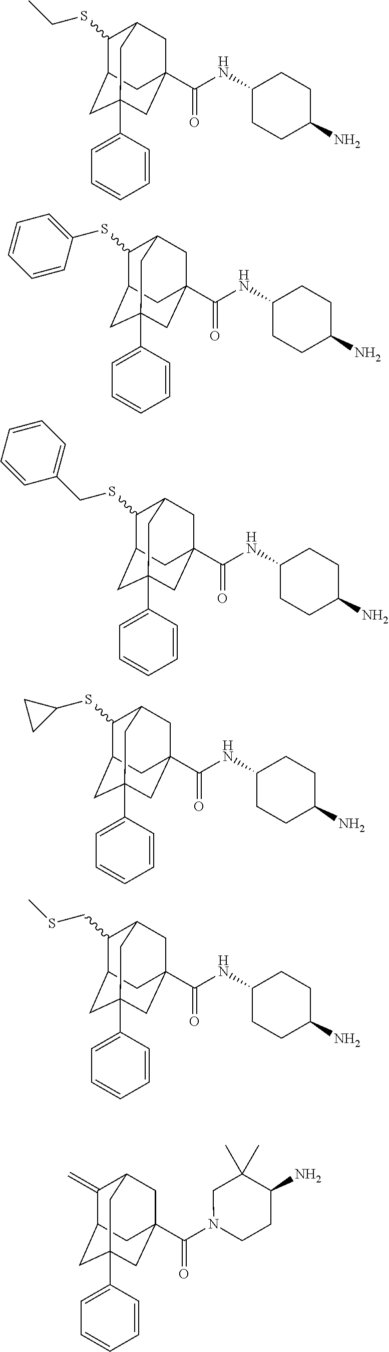

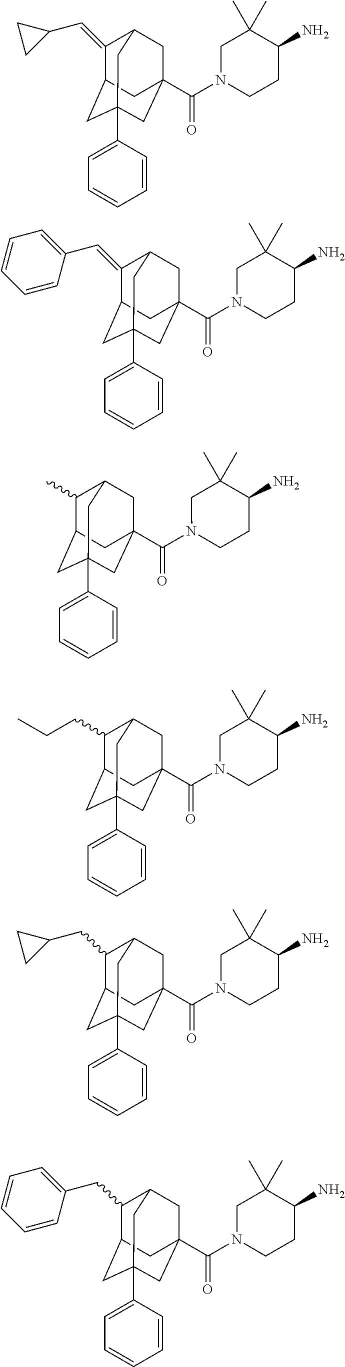

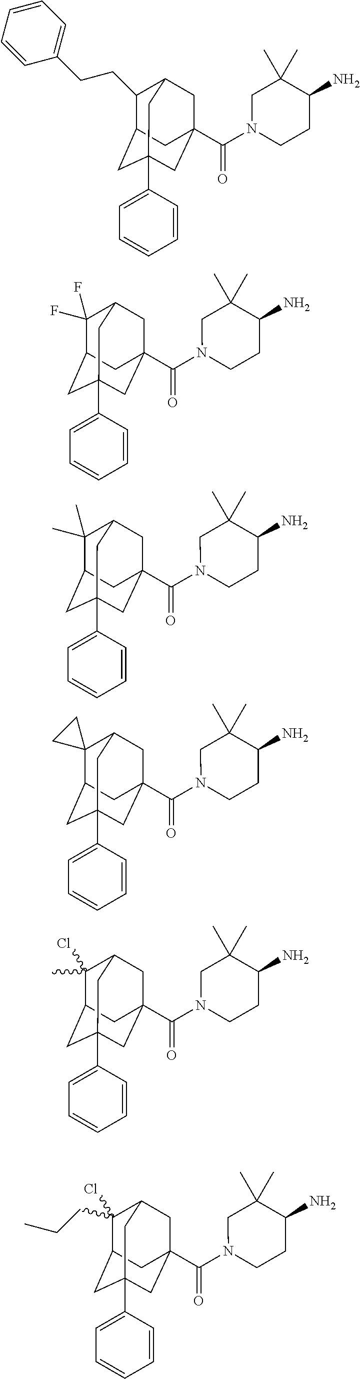

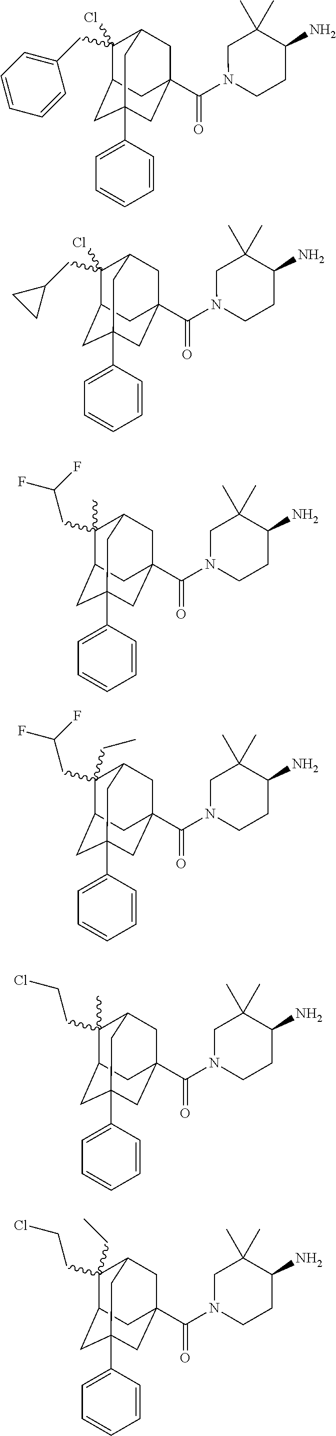

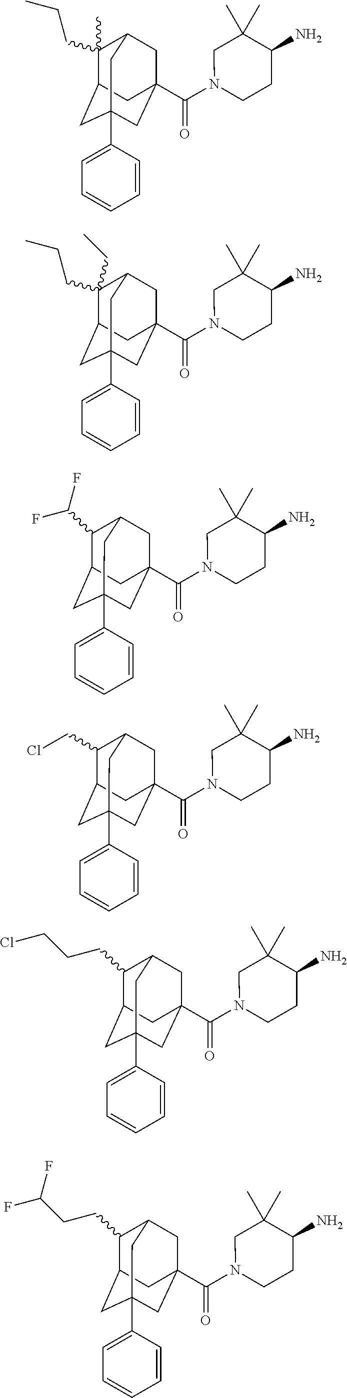

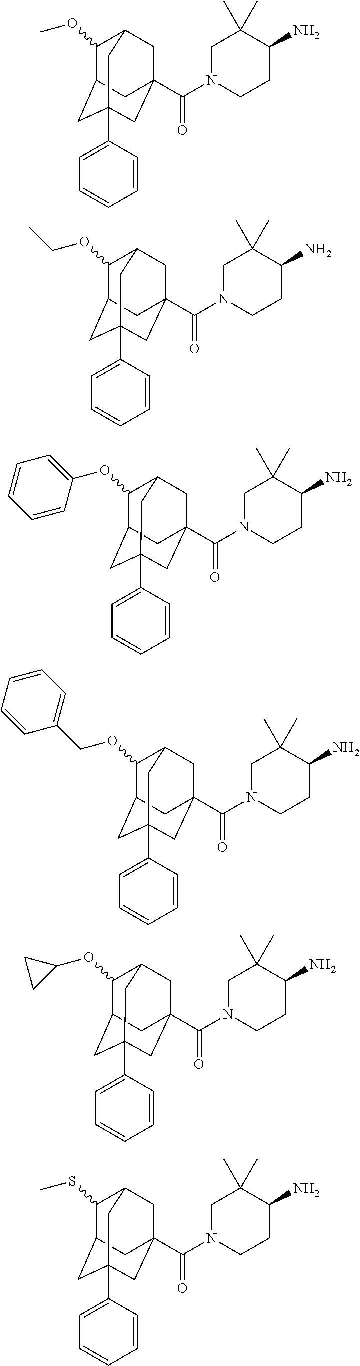

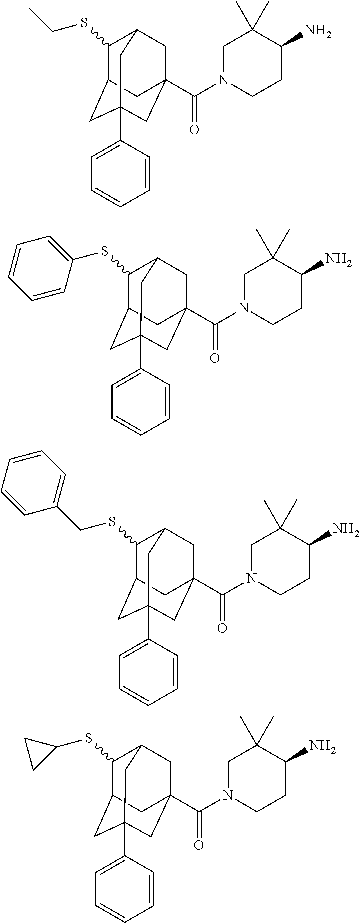

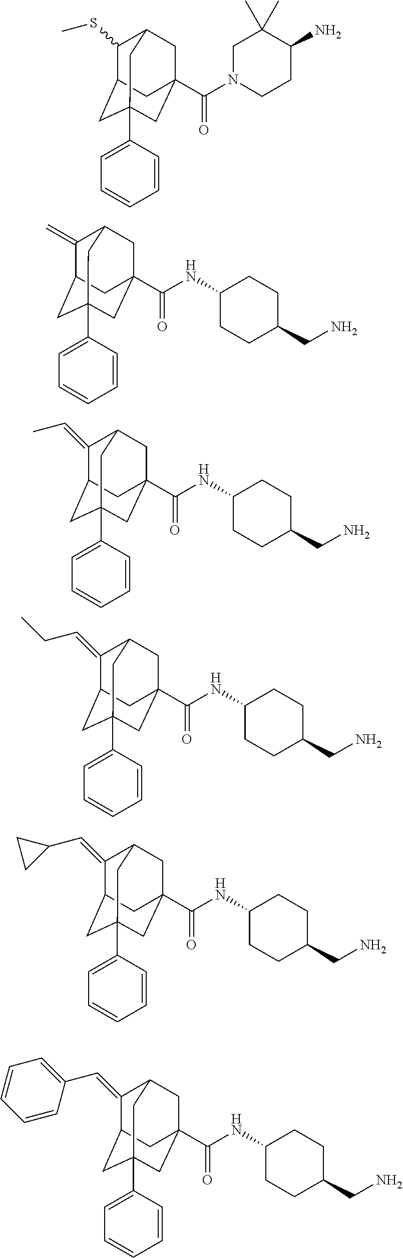

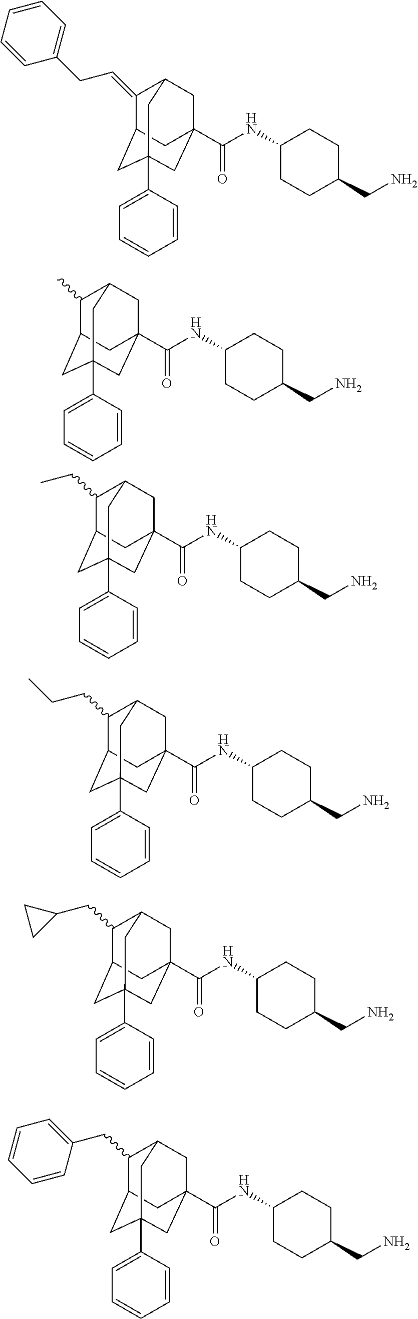

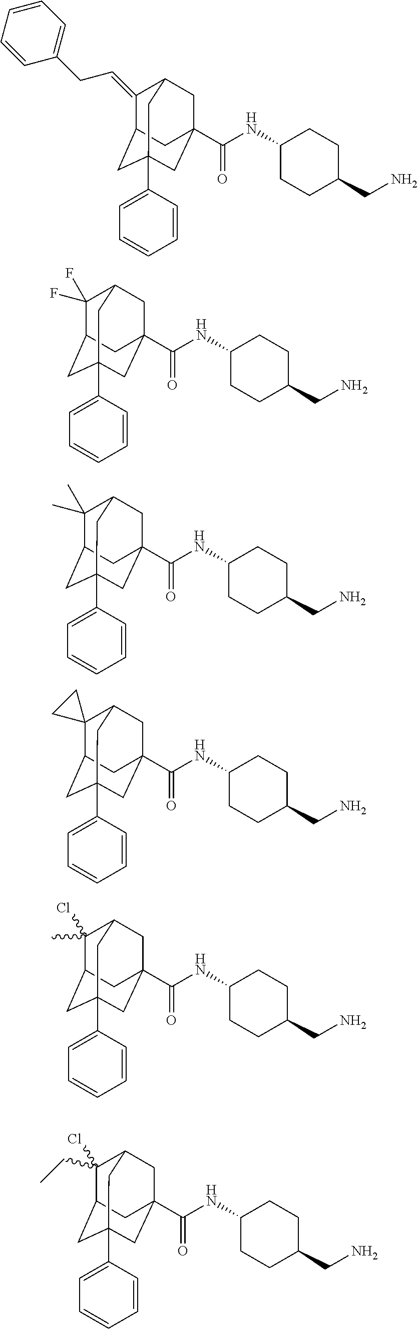

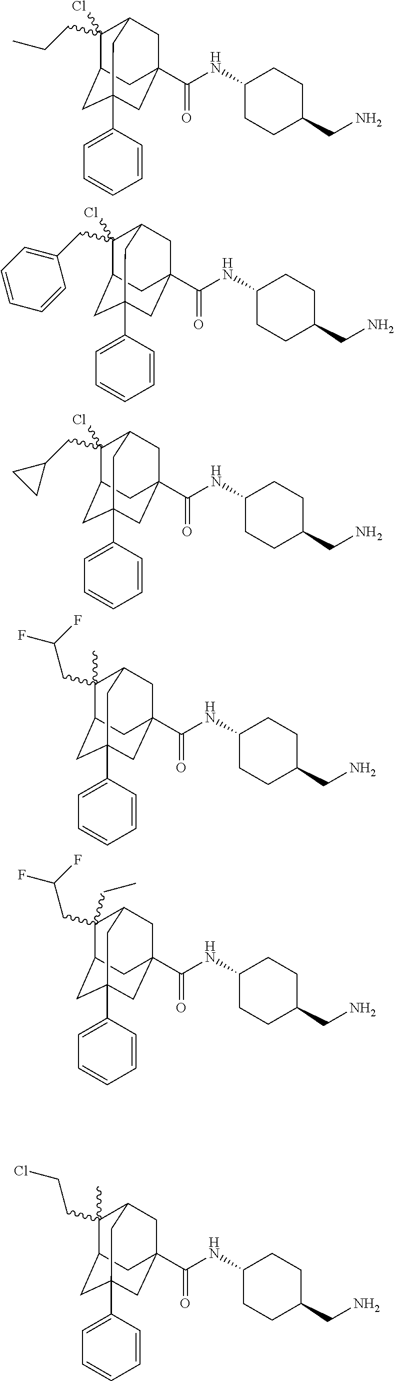

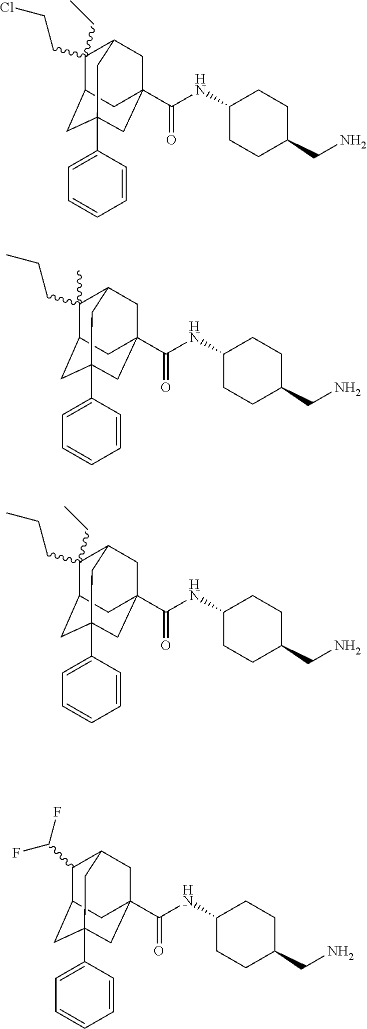

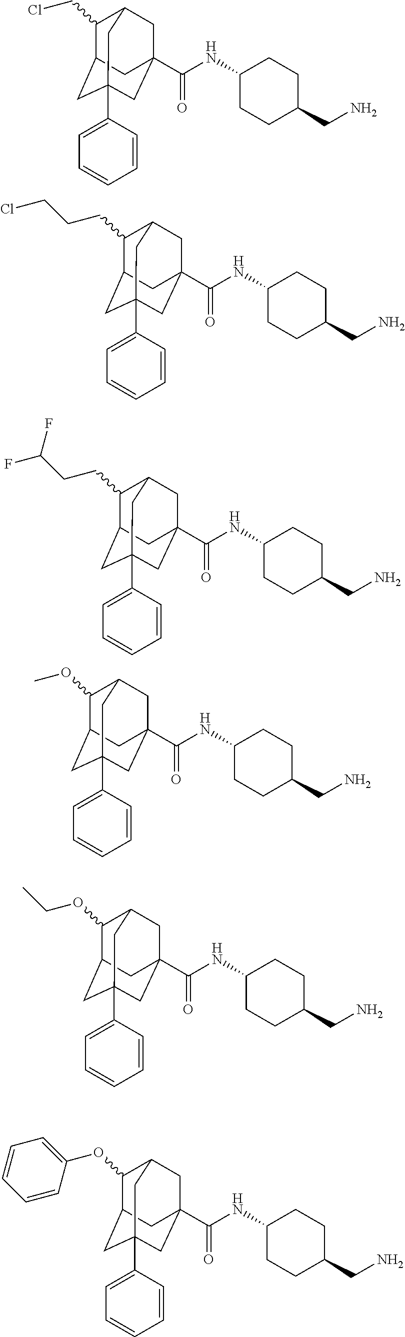

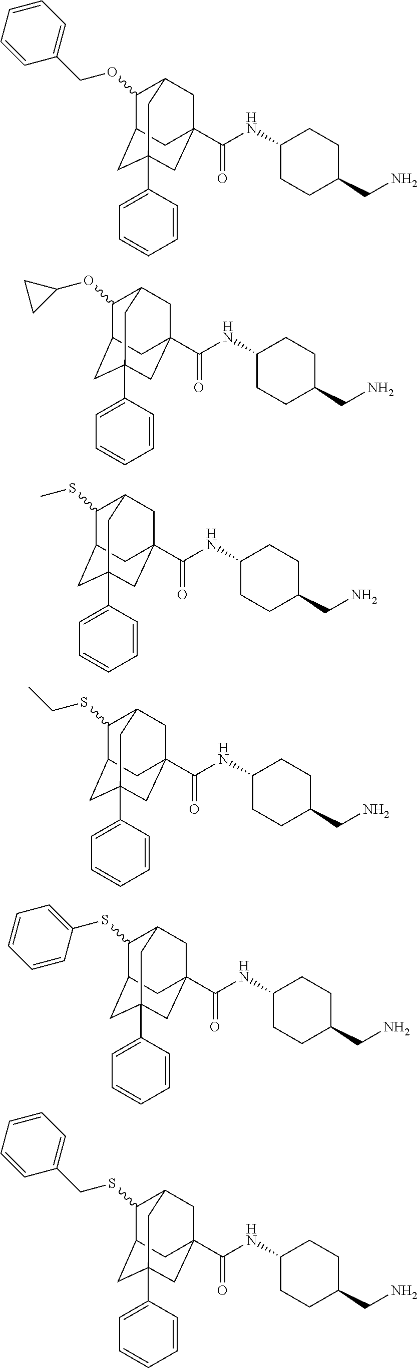

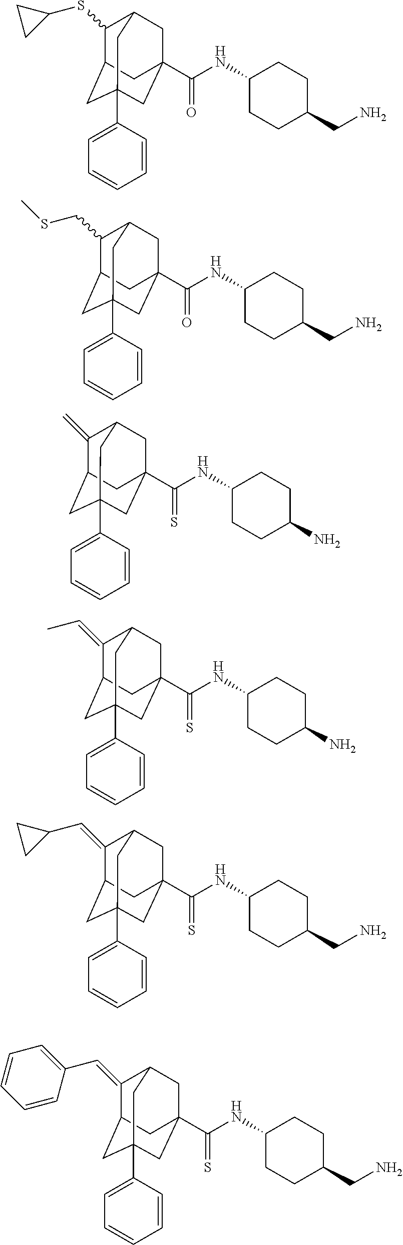

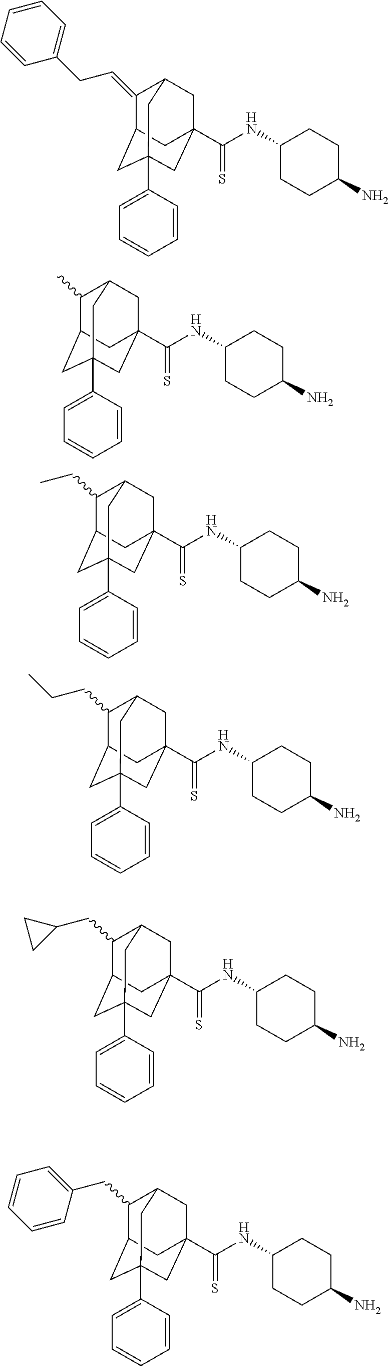

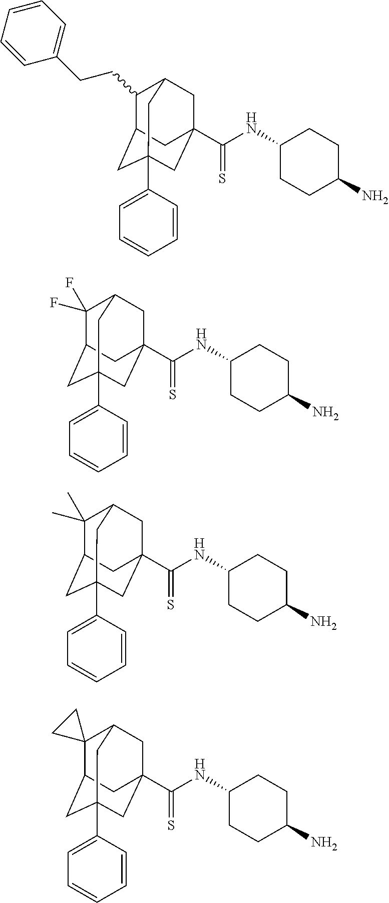

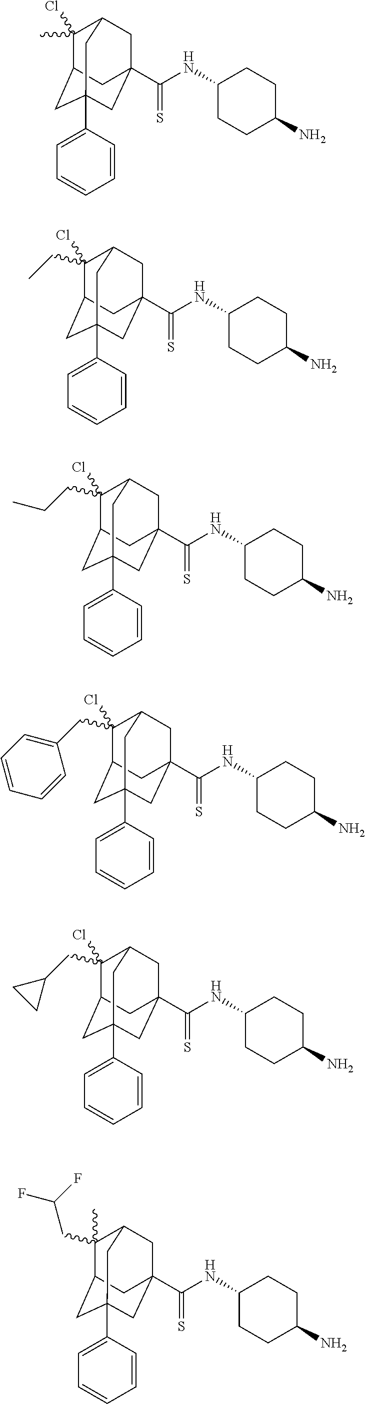

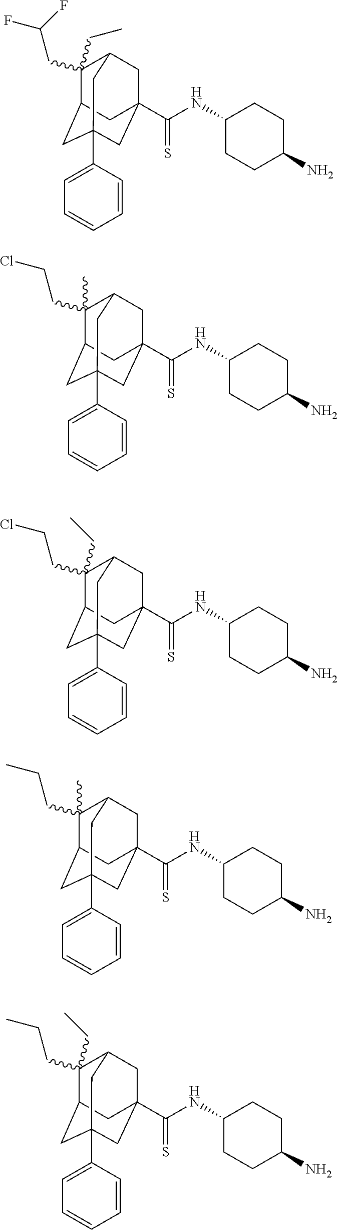

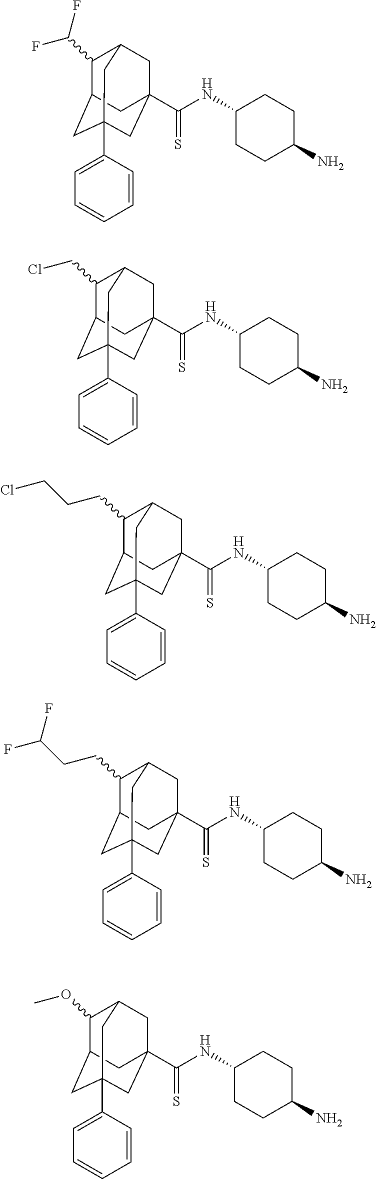 [SMILES: N[C@H]1CC[C@H](NC(=S)C23CC4CC(c5ccccc5)(CC(C2)C4C(F)F)C3)CC1 ; N[C@H]1CC[C@H](NC(=S)C23CC4CC(c5ccccc5)(CC(C2)C4CCl)C3)CC1 ; N[C@H]1CC[C@H](NC(=S)C23CC4CC(c5ccccc5)(CC(C2)C4CCCCl)C3)CC1 ; N[C@H]1CC[C@H](NC(=S)C23CC4CC(c5ccccc5)(CC(C2)C4CCC(F)F)C3)CC1 ; COC1C2CC3(C(=S)N[C@H]4CC[C@H](N)CC4)CC1CC(c1ccccc1)(C2)C3]

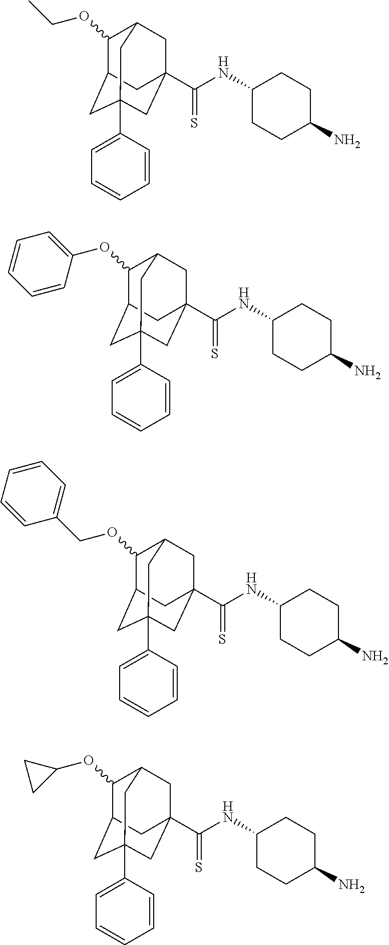 [SMILES: CCOC1C2CC3(C(=S)N[C@H]4CC[C@H](N)CC4)CC1CC(c1ccccc1)(C2)C3 ; N[C@H]1CC[C@H](NC(=S)C23CC4CC(c5ccccc5)(CC(C2)C4Oc2ccccc2)C3)CC1 ; N[C@H]1CC[C@H](NC(=S)C23CC4CC(c5ccccc5)(CC(C2)C4OCc2ccccc2)C3)CC1 ; N[C@H]1CC[C@H](NC(=S)C23CC4CC(c5ccccc5)(CC(C2)C4OC2CC2)C3)CC1]

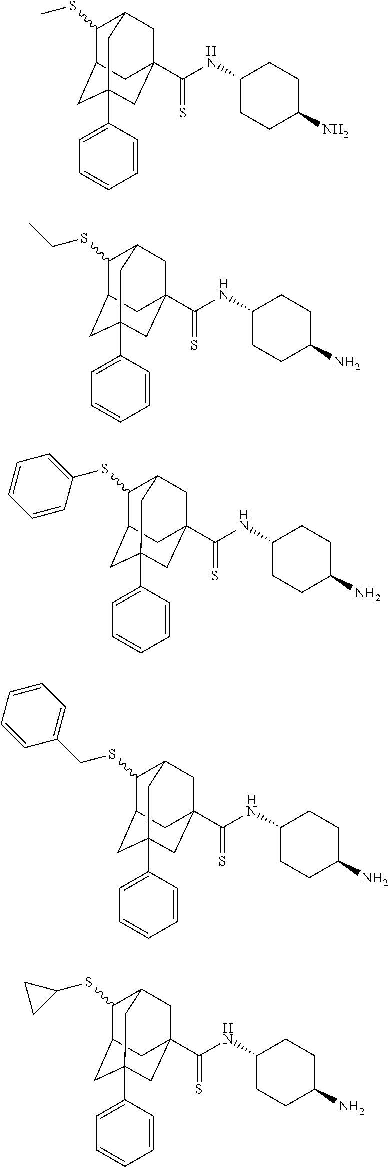 [SMILES: CSC1C2CC3(C(=S)N[C@H]4CC[C@H](N)CC4)CC1CC(c1ccccc1)(C2)C3 ; CCSC1C2CC3(C(=S)N[C@H]4CC[C@H](N)CC4)CC1CC(c1ccccc1)(C2)C3 ; N[C@H]1CC[C@H](NC(=S)C23CC4CC(c5ccccc5)(CC(C2)C4Sc2ccccc2)C3)CC1 ; N[C@H]1CC[C@H](NC(=S)C23CC4CC(c5ccccc5)(CC(C2)C4SCc2ccccc2)C3)CC1 ; N[C@H]1CC[C@H](NC(=S)C23CC4CC(c5ccccc5)(CC(C2)C4SC2CC2)C3)CC1]

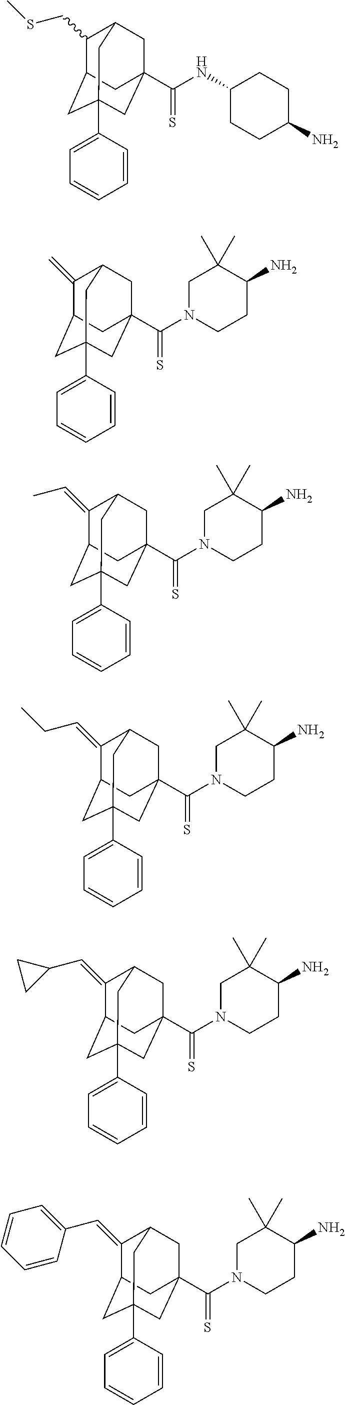 [SMILES: CSCC1C2CC3(C(=S)N[C@H]4CC[C@H](N)CC4)CC1CC(c1ccccc1)(C2)C3 ; C=C1C2CC3(C(=S)N4CC[C@H](N)C(C)(C)C4)CC1CC(c1ccccc1)(C2)C3 ; CC=C1C2CC3(C(=S)N4CC[C@H](N)C(C)(C)C4)CC1CC(c1ccccc1)(C2)C3 ; CCC=C1C2CC3(C(=S)N4CC[C@H](N)C(C)(C)C4)CC1CC(c1ccccc1)(C2)C3 ; CC1(C)CN(C(=S)C23CC4CC(c5ccccc5)(CC(C2)C4=CC2CC2)C3)CC[C@@H]1N ; CC1(C)CN(C(=S)C23CC4CC(c5ccccc5)(CC(C2)C4=Cc2ccccc2)C3)CC[C@@H]1N]

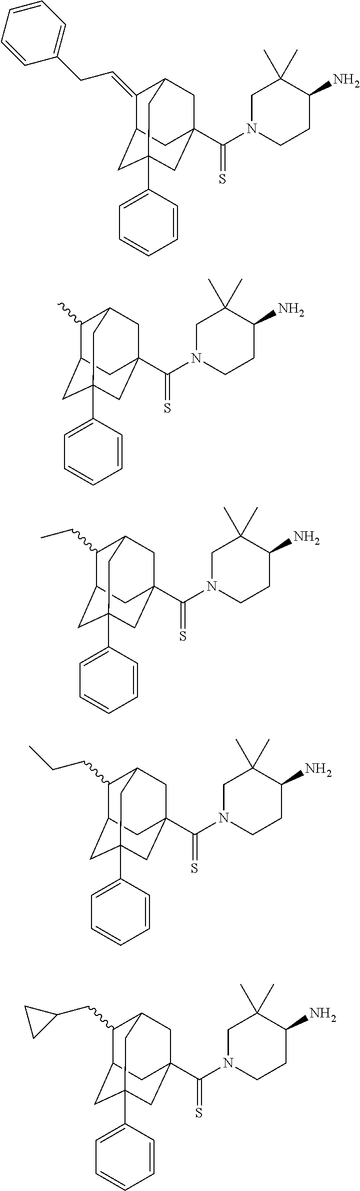 [SMILES: CC1(C)CN(C(=S)C23CC4CC(c5ccccc5)(CC(C2)C4=CCc2ccccc2)C3)CC[C@@H]1N ; CC1C2CC3(C(=S)N4CC[C@H](N)C(C)(C)C4)CC1CC(c1ccccc1)(C2)C3 ; CCC1C2CC3(C(=S)N4CC[C@H](N)C(C)(C)C4)CC1CC(c1ccccc1)(C2)C3 ; CCCC1C2CC3(C(=S)N4CC[C@H](N)C(C)(C)C4)CC1CC(c1ccccc1)(C2)C3 ; CC1(C)CN(C(=S)C23CC4CC(c5ccccc5)(CC(C2)C4CC2CC2)C3)CC[C@@H]1N]

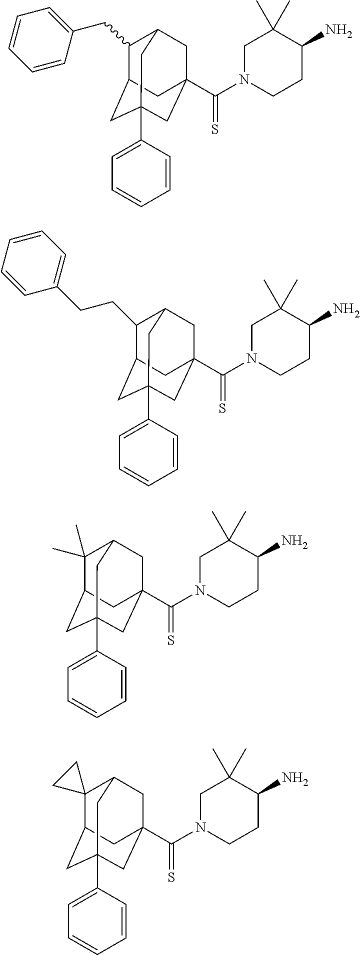 [SMILES: CC1(C)CN(C(=S)C23CC4CC(c5ccccc5)(CC(C2)C4Cc2ccccc2)C3)CC[C@@H]1N ; CC1(C)CN(C(=S)C23CC4CC(c5ccccc5)(CC(C2)C4CCc2ccccc2)C3)CC[C@@H]1N ; CC1(C)C2CC3(C(=S)N4CC[C@H](N)C(C)(C)C4)CC1CC(c1ccccc1)(C2)C3 ; CC1(C)CN(C(=S)C23CC4CC(c5ccccc5)(CC(C2)C42CC2)C3)CC[C@@H]1N]

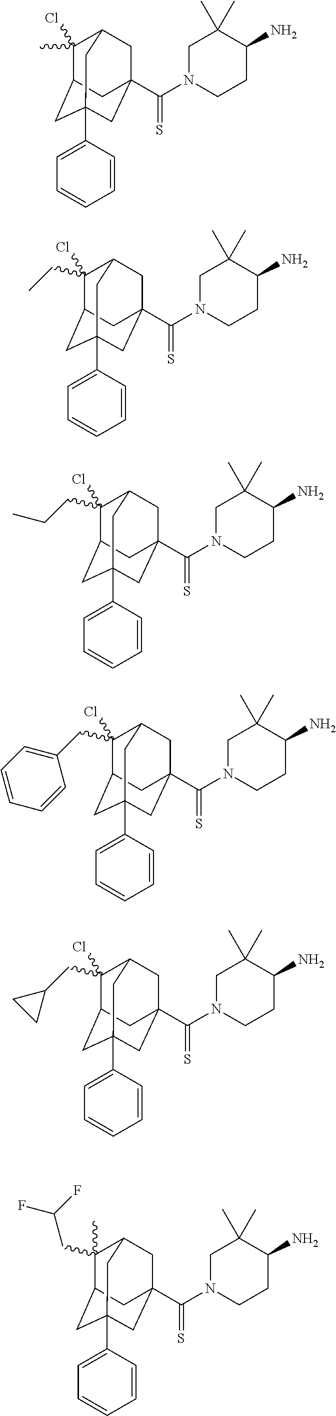

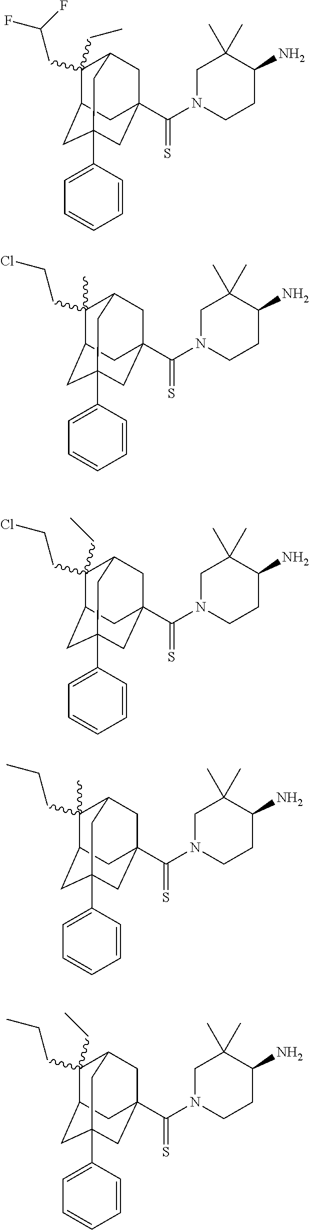

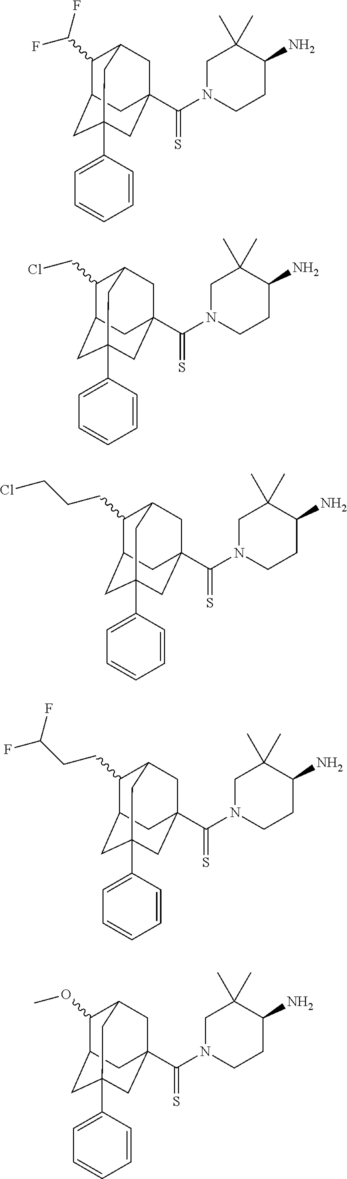 [SMILES: CC1(C)CN(C(=S)C23CC4CC(c5ccccc5)(CC(C2)C4C(F)F)C3)CC[C@@H]1N ; CC1(C)CN(C(=S)C23CC4CC(c5ccccc5)(CC(C2)C4CCl)C3)CC[C@@H]1N ; CC1(C)CN(C(=S)C23CC4CC(c5ccccc5)(CC(C2)C4CCCCl)C3)CC[C@@H]1N ; CC1(C)CN(C(=S)C23CC4CC(c5ccccc5)(CC(C2)C4CCC(F)F)C3)CC[C@@H]1N ; COC1C2CC3(C(=S)N4CC[C@H](N)C(C)(C)C4)CC1CC(c1ccccc1)(C2)C3]

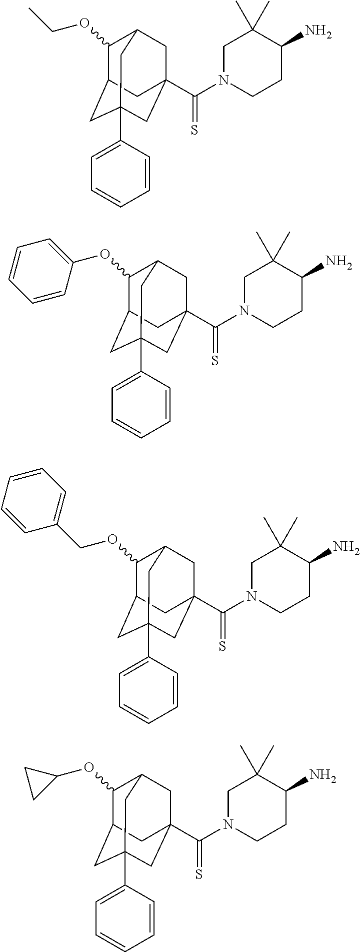 [SMILES: CCOC1C2CC3(C(=S)N4CC[C@H](N)C(C)(C)C4)CC1CC(c1ccccc1)(C2)C3 ; CC1(C)CN(C(=S)C23CC4CC(c5ccccc5)(CC(C2)C4Oc2ccccc2)C3)CC[C@@H]1N ; CC1(C)CN(C(=S)C23CC4CC(c5ccccc5)(CC(C2)C4OCc2ccccc2)C3)CC[C@@H]1N ; CC1(C)CN(C(=S)C23CC4CC(c5ccccc5)(CC(C2)C4OC2CC2)C3)CC[C@@H]1N]

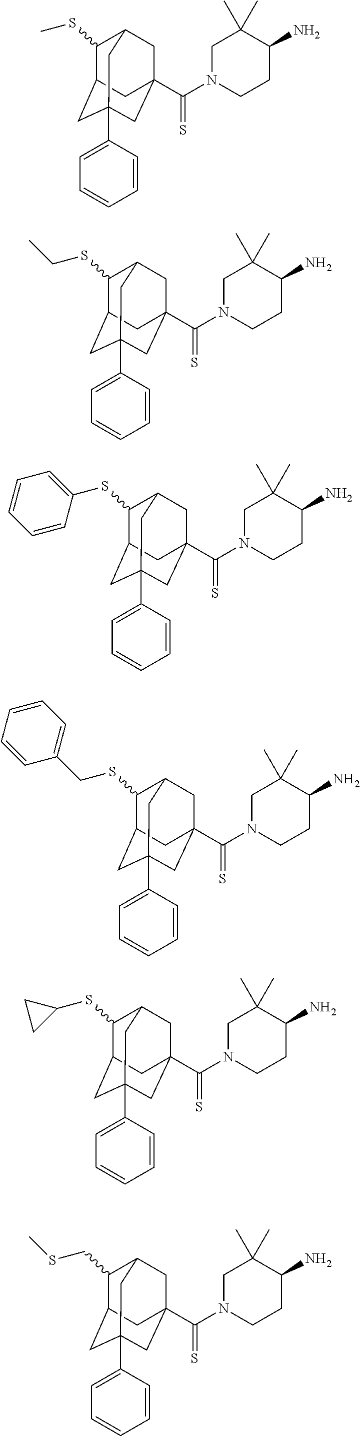 [SMILES: CSC1C2CC3(C(=S)N4CC[C@H](N)C(C)(C)C4)CC1CC(c1ccccc1)(C2)C3 ; CCSC1C2CC3(C(=S)N4CC[C@H](N)C(C)(C)C4)CC1CC(c1ccccc1)(C2)C3 ; CC1(C)CN(C(=S)C23CC4CC(c5ccccc5)(CC(C2)C4Sc2ccccc2)C3)CC[C@@H]1N ; CC1(C)CN(C(=S)C23CC4CC(c5ccccc5)(CC(C2)C4SCc2ccccc2)C3)CC[C@@H]1N ; CC1(C)CN(C(=S)C23CC4CC(c5ccccc5)(CC(C2)C4SC2CC2)C3)CC[C@@H]1N ; CSCC1C2CC3(C(=S)N4CC[C@H](N)C(C)(C)C4)CC1CC(c1ccccc1)(C2)C3]

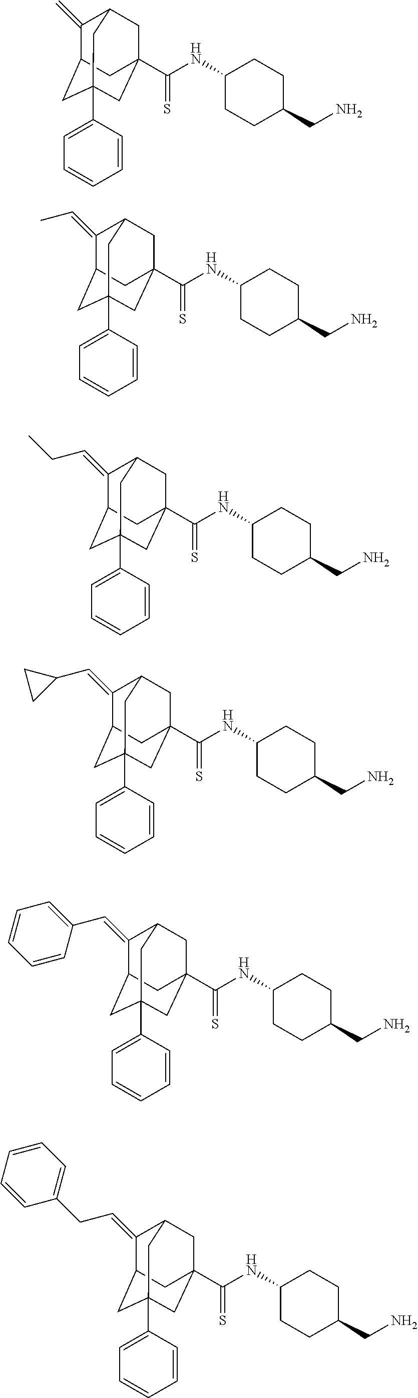

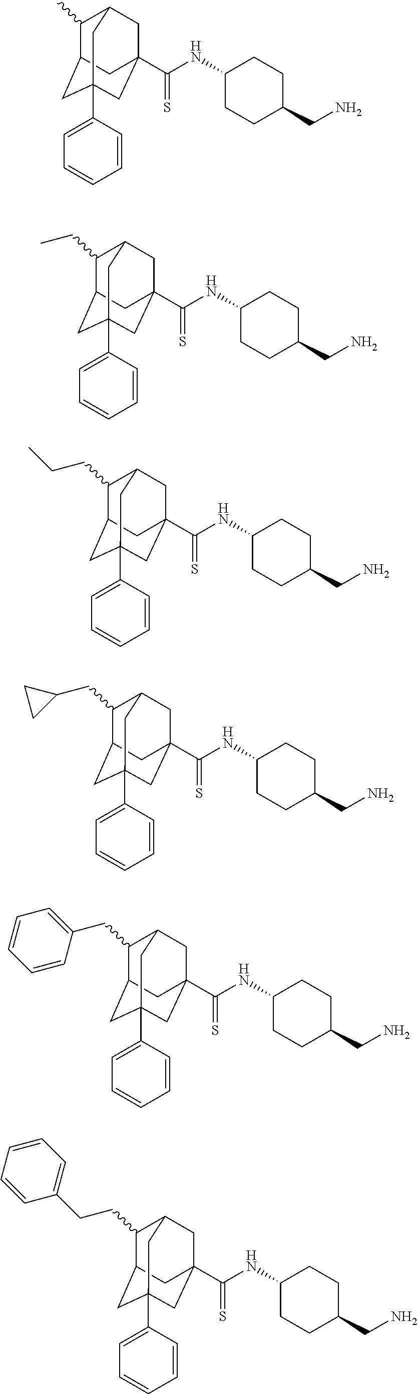 [SMILES: CC1C2CC3(C(=S)N[C@H]4CC[C@H](CN)CC4)CC1CC(c1ccccc1)(C2)C3 ; CCC1C2CC3(C(=S)N[C@H]4CC[C@H](CN)CC4)CC1CC(c1ccccc1)(C2)C3 ; CCCC1C2CC3(C(=S)N[C@H]4CC[C@H](CN)CC4)CC1CC(c1ccccc1)(C2)C3 ; NC[C@H]1CC[C@H](NC(=S)C23CC4CC(c5ccccc5)(CC(C2)C4CC2CC2)C3)CC1 ; NC[C@H]1CC[C@H](NC(=S)C23CC4CC(c5ccccc5)(CC(C2)C4Cc2ccccc2)C3)CC1 ; NC[C@H]1CC[C@H](NC(=S)C23CC4CC(c5ccccc5)(CC(C2)C4CCc2ccccc2)C3)CC1]

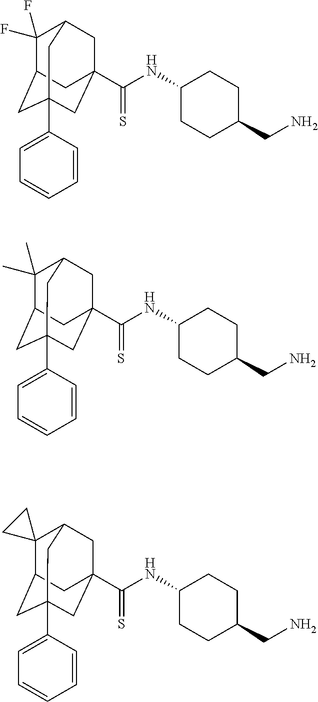 [SMILES: NC[C@H]1CC[C@H](NC(=S)C23CC4CC(c5ccccc5)(CC(C2)C4(F)F)C3)CC1 ; CC1(C)C2CC3(C(=S)N[C@H]4CC[C@H](CN)CC4)CC1CC(c1ccccc1)(C2)C3 ; NC[C@H]1CC[C@H](NC(=S)C23CC4CC(c5ccccc5)(CC(C2)C42CC2)C3)CC1]

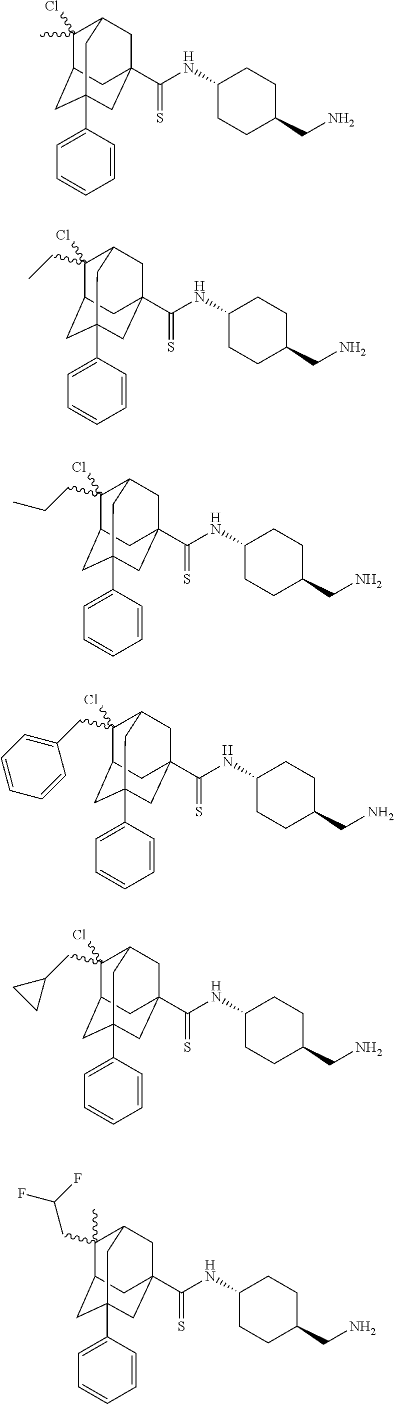

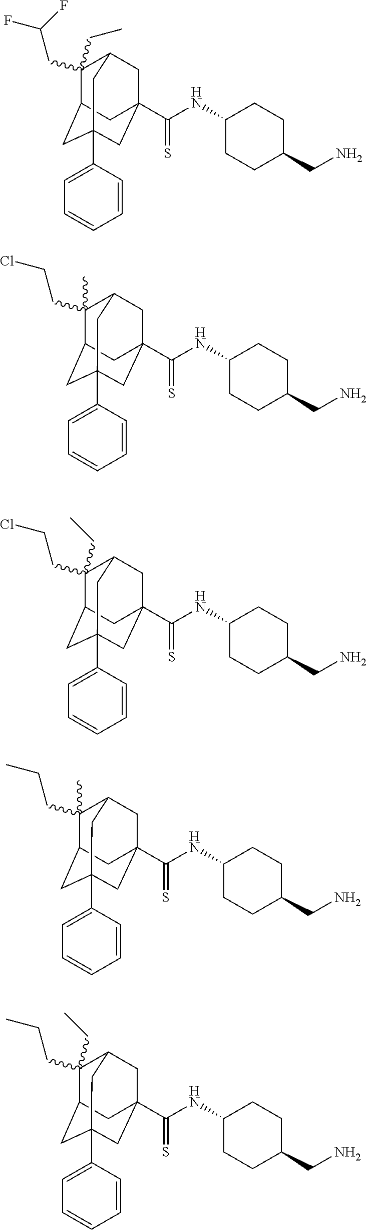

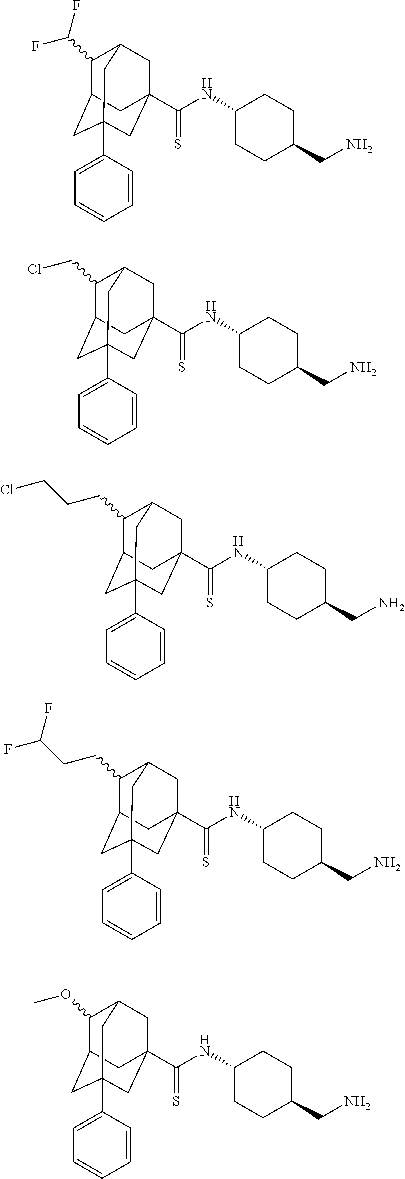 [SMILES: NC[C@H]1CC[C@H](NC(=S)C23CC4CC(c5ccccc5)(CC(C2)C4C(F)F)C3)CC1 ; NC[C@H]1CC[C@H](NC(=S)C23CC4CC(c5ccccc5)(CC(C2)C4CCl)C3)CC1 ; NC[C@H]1CC[C@H](NC(=S)C23CC4CC(c5ccccc5)(CC(C2)C4CCCCl)C3)CC1 ; NC[C@H]1CC[C@H](NC(=S)C23CC4CC(c5ccccc5)(CC(C2)C4CCC(F)F)C3)CC1 ; COC1C2CC3(C(=S)N[C@H]4CC[C@H](CN)CC4)CC1CC(c1ccccc1)(C2)C3]

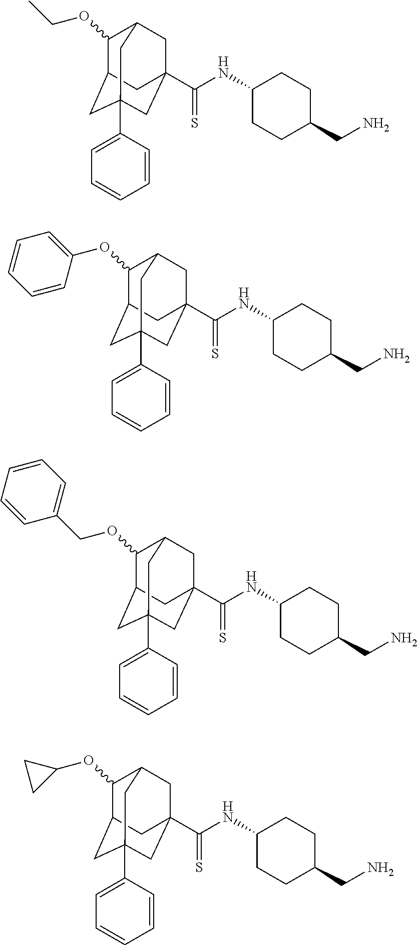 [SMILES: CCOC1C2CC3(C(=S)N[C@H]4CC[C@H](CN)CC4)CC1CC(c1ccccc1)(C2)C3 ; NC[C@H]1CC[C@H](NC(=S)C23CC4CC(c5ccccc5)(CC(C2)C4Oc2ccccc2)C3)CC1 ; NC[C@H]1CC[C@H](NC(=S)C23CC4CC(c5ccccc5)(CC(C2)C4OCc2ccccc2)C3)CC1 ; NC[C@H]1CC[C@H](NC(=S)C23CC4CC(c5ccccc5)(CC(C2)C4OC2CC2)C3)CC1]

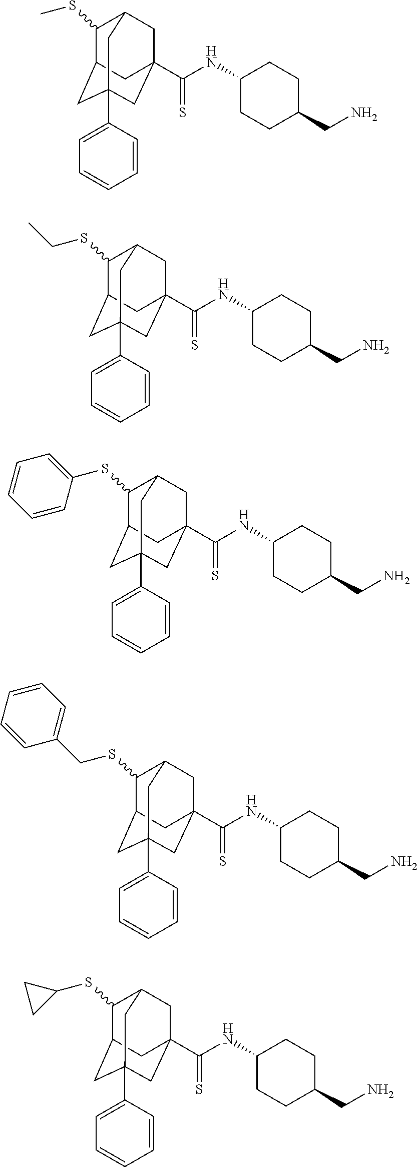 [SMILES: CSC1C2CC3(C(=S)N[C@H]4CC[C@H](CN)CC4)CC1CC(c1ccccc1)(C2)C3 ; CCSC1C2CC3(C(=S)N[C@H]4CC[C@H](CN)CC4)CC1CC(c1ccccc1)(C2)C3 ; NC[C@H]1CC[C@H](NC(=S)C23CC4CC(c5ccccc5)(CC(C2)C4Sc2ccccc2)C3)CC1 ; NC[C@H]1CC[C@H](NC(=S)C23CC4CC(c5ccccc5)(CC(C2)C4SCc2ccccc2)C3)CC1 ; NC[C@H]1CC[C@H](NC(=S)C23CC4CC(c5ccccc5)(CC(C2)C4SC2CC2)C3)CC1]

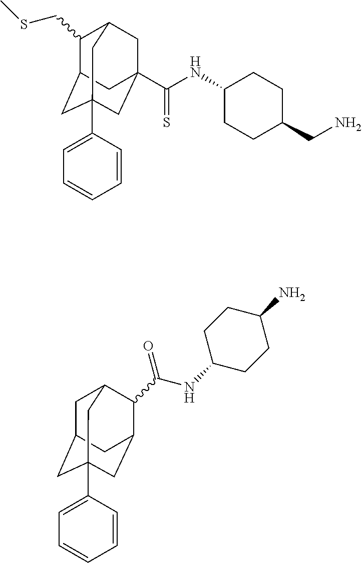 [SMILES: CSCC1C2CC3(C(=S)N[C@H]4CC[C@H](CN)CC4)CC1CC(c1ccccc1)(C2)C3 ; N[C@H]1CC[C@H](NC(=O)C2C3CC4CC2CC(c2ccccc2)(C4)C3)CC1]

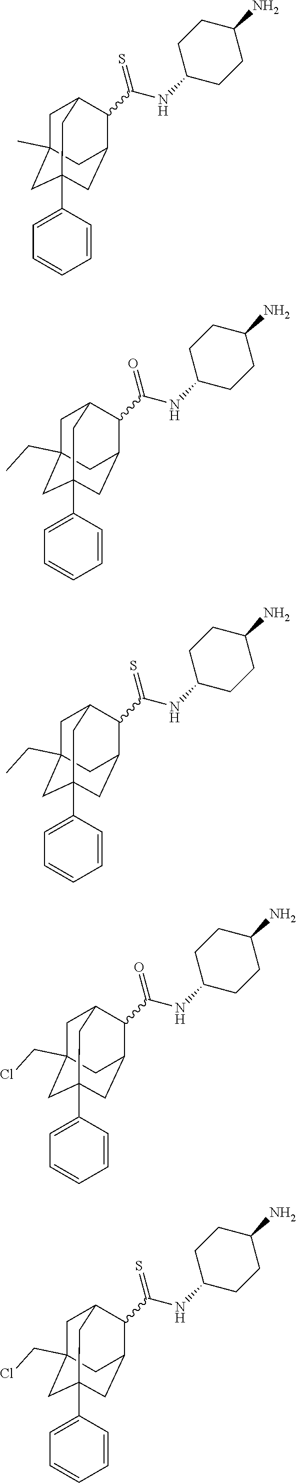 [SMILES: CC12CC3CC(c4ccccc4)(CC(C1)C3C(=S)N[C@H]1CC[C@H](N)CC1)C2 ; CCC12CC3CC(c4ccccc4)(CC(C1)C3C(=O)N[C@H]1CC[C@H](N)CC1)C2 ; CCC12CC3CC(c4ccccc4)(CC(C1)C3C(=S)N[C@H]1CC[C@H](N)CC1)C2 ; N[C@H]1CC[C@H](NC(=O)C2C3CC4(CCl)CC2CC(c2ccccc2)(C3)C4)CC1 ; N[C@H]1CC[C@H](NC(=S)C2C3CC4(CCl)CC2CC(c2ccccc2)(C3)C4)CC1]

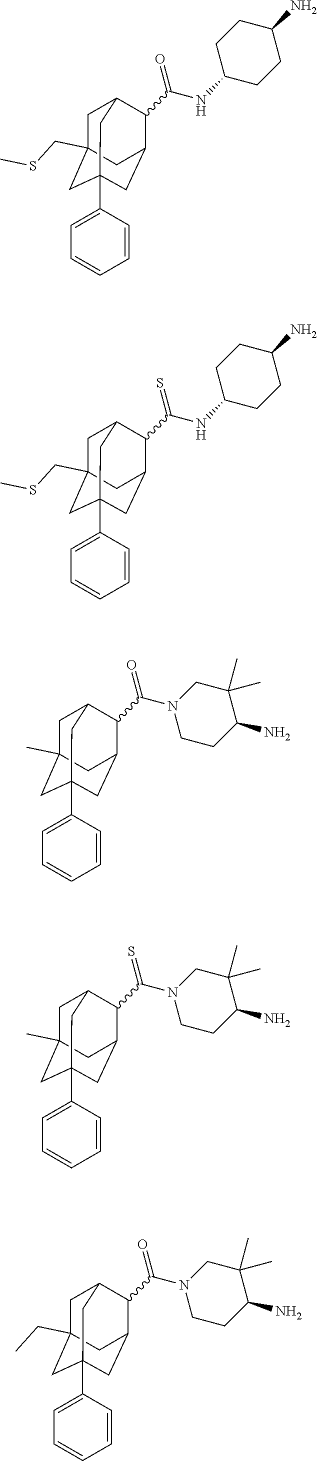 [SMILES: CSCC12CC3CC(c4ccccc4)(CC(C1)C3C(=O)N[C@H]1CC[C@H](N)CC1)C2 ; CSCC12CC3CC(c4ccccc4)(CC(C1)C3C(=S)N[C@H]1CC[C@H](N)CC1)C2 ; CC12CC3CC(c4ccccc4)(CC(C1)C3C(=O)N1CC[C@H](N)C(C)(C)C1)C2 ; CC12CC3CC(c4ccccc4)(CC(C1)C3C(=S)N1CC[C@H](N)C(C)(C)C1)C2 ; CCC12CC3CC(c4ccccc4)(CC(C1)C3C(=O)N1CC[C@H](N)C(C)(C)C1)C2]

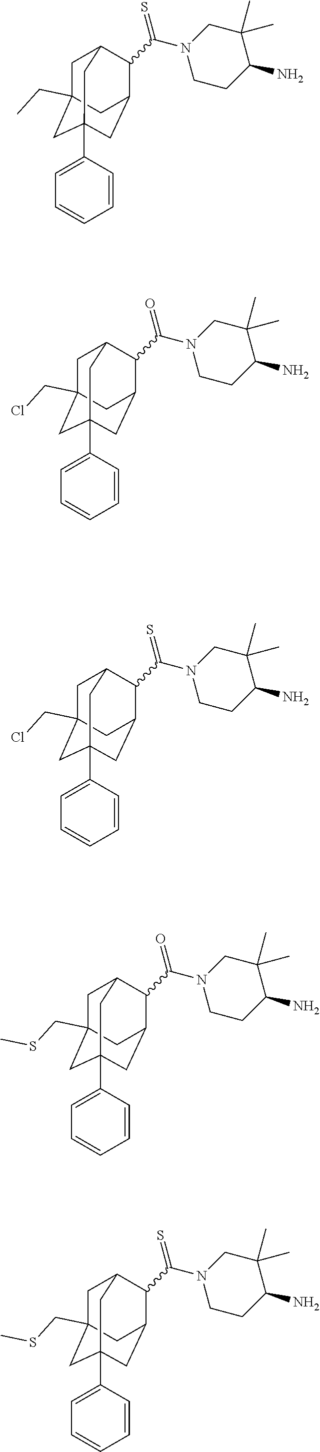 [SMILES: CCC12CC3CC(c4ccccc4)(CC(C1)C3C(=S)N1CC[C@H](N)C(C)(C)C1)C2 ; CC1(C)CN(C(=O)C2C3CC4(CCl)CC2CC(c2ccccc2)(C3)C4)CC[C@@H]1N ; CC1(C)CN(C(=S)C2C3CC4(CCl)CC2CC(c2ccccc2)(C3)C4)CC[C@@H]1N ; CSCC12CC3CC(c4ccccc4)(CC(C1)C3C(=O)N1CC[C@H](N)C(C)(C)C1)C2 ; CSCC12CC3CC(c4ccccc4)(CC(C1)C3C(=S)N1CC[C@H](N)C(C)(C)C1)C2]

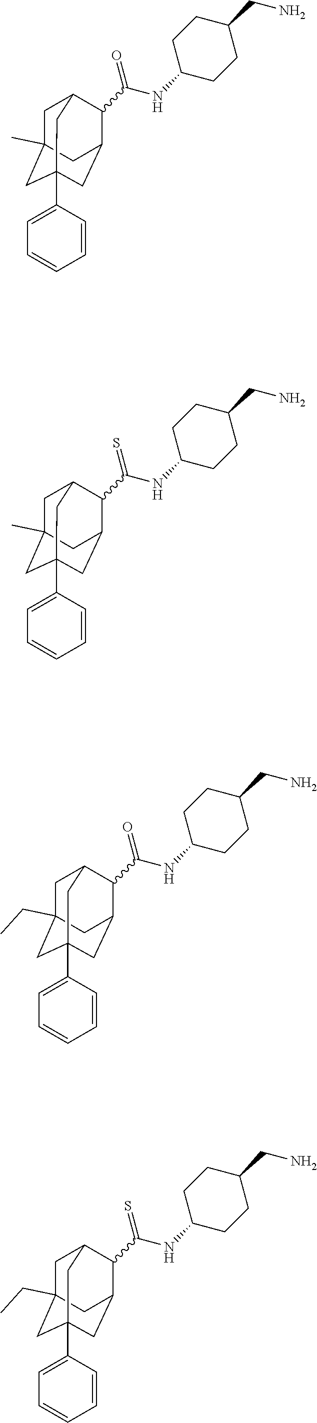 [SMILES: CC12CC3CC(c4ccccc4)(CC(C1)C3C(=O)N[C@H]1CC[C@H](CN)CC1)C2 ; CC12CC3CC(c4ccccc4)(CC(C1)C3C(=S)N[C@H]1CC[C@H](CN)CC1)C2 ; CCC12CC3CC(c4ccccc4)(CC(C1)C3C(=O)N[C@H]1CC[C@H](CN)CC1)C2 ; CCC12CC3CC(c4ccccc4)(CC(C1)C3C(=S)N[C@H]1CC[C@H](CN)CC1)C2]

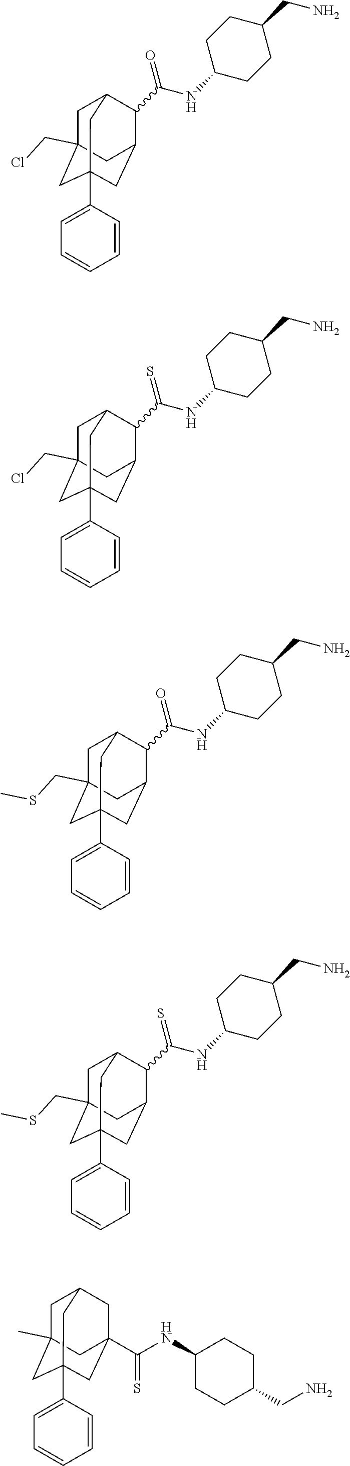 [SMILES: NC[C@H]1CC[C@H](NC(=O)C2C3CC4(CCl)CC2CC(c2ccccc2)(C3)C4)CC1 ; NC[C@H]1CC[C@H](NC(=S)C2C3CC4(CCl)CC2CC(c2ccccc2)(C3)C4)CC1 ; CSCC12CC3CC(c4ccccc4)(CC(C1)C3C(=O)N[C@H]1CC[C@H](CN)CC1)C2 ; CSCC12CC3CC(c4ccccc4)(CC(C1)C3C(=S)N[C@H]1CC[C@H](CN)CC1)C2 ; CC12CC3CC(C(=S)N[C@H]4CC[C@H](CN)CC4)(C1)CC(c1ccccc1)(C3)C2]

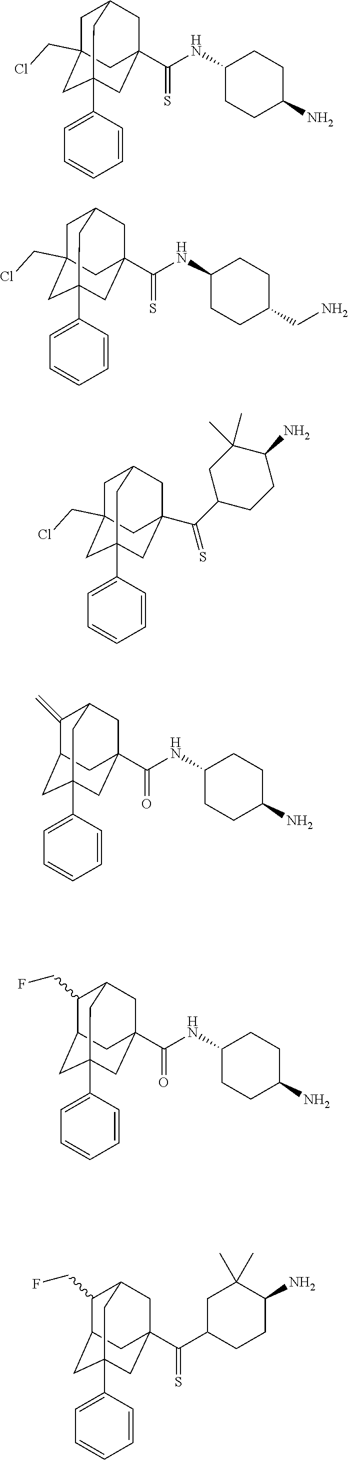

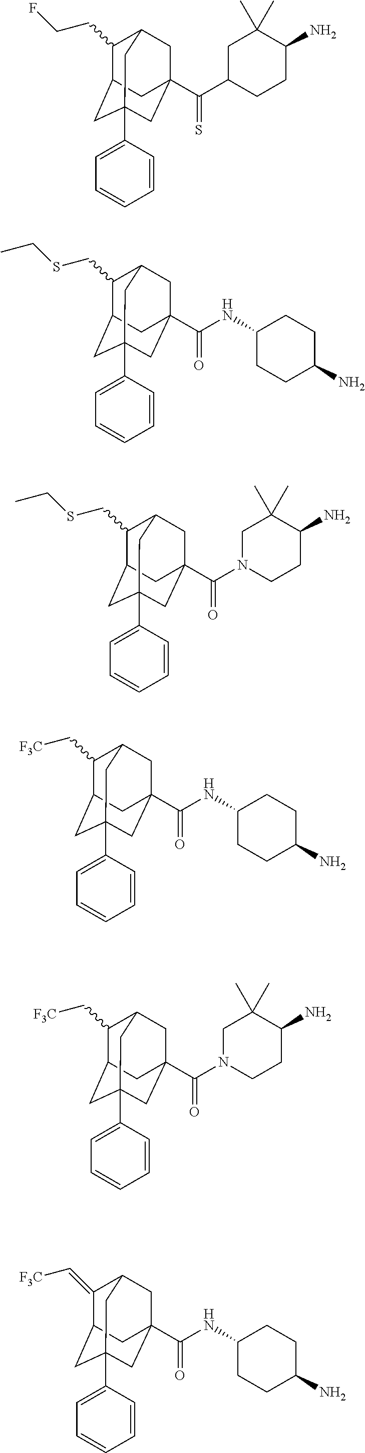 [SMILES: CC1(C)CC(C(=S)C23CC4CC(c5ccccc5)(CC(C2)C4CCF)C3)CC[C@@H]1N ; CCSCC1C2CC3(C(=O)N[C@H]4CC[C@H](N)CC4)CC1CC(c1ccccc1)(C2)C3 ; CCSCC1C2CC3(C(=O)N4CC[C@H](N)C(C)(C)C4)CC1CC(c1ccccc1)(C2)C3 ; N[C@H]1CC[C@H](NC(=O)C23CC4CC(c5ccccc5)(CC(C2)C4CC(F)(F)F)C3)CC1 ; CC1(C)CN(C(=O)C23CC4CC(c5ccccc5)(CC(C2)C4CC(F)(F)F)C3)CC[C@@H]1N ; N[C@H]1CC[C@H](NC(=O)C23CC4CC(c5ccccc5)(CC(C2)C4=CC(F)(F)F)C3)CC1]

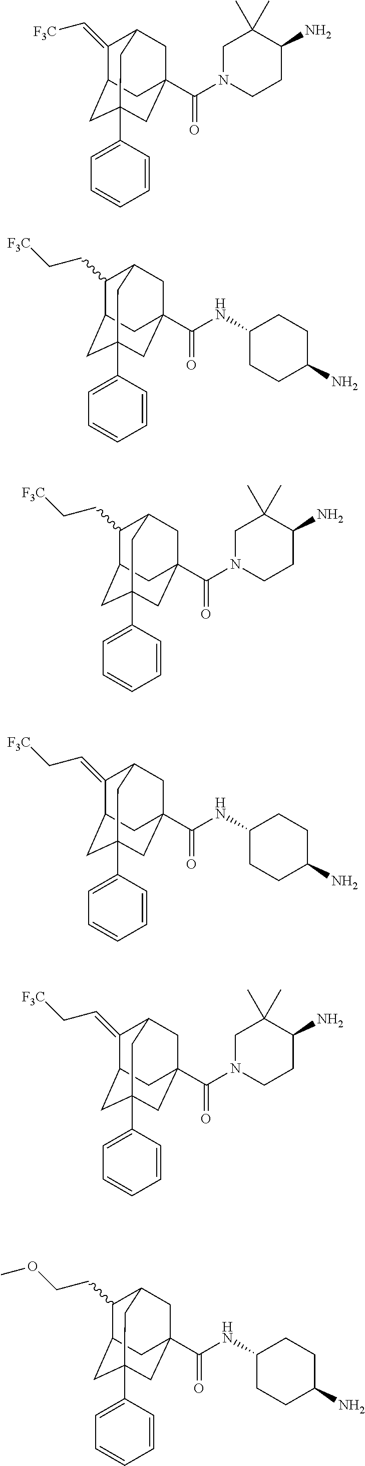 [SMILES: CC1(C)CN(C(=O)C23CC4CC(c5ccccc5)(CC(C2)C4=CC(F)(F)F)C3)CC[C@@H]1N ; N[C@H]1CC[C@H](NC(=O)C23CC4CC(c5ccccc5)(CC(C2)C4CCC(F)(F)F)C3)CC1 ; CC1(C)CN(C(=O)C23CC4CC(c5ccccc5)(CC(C2)C4CCC(F)(F)F)C3)CC[C@@H]1N ; N[C@H]1CC[C@H](NC(=O)C23CC4CC(c5ccccc5)(CC(C2)C4=CCC(F)(F)F)C3)CC1 ; CC1(C)CN(C(=O)C23CC4CC(c5ccccc5)(CC(C2)C4=CCC(F)(F)F)C3)CC[C@@H]1N ; COCCC1C2CC3(C(=O)N[C@H]4CC[C@H](N)CC4)CC1CC(c1ccccc1)(C2)C3]

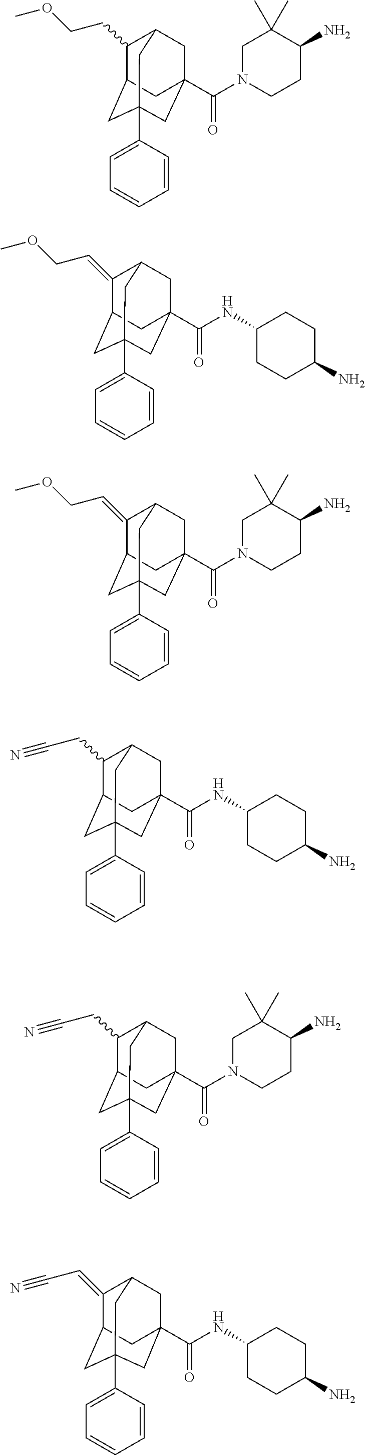 [SMILES: COCCC1C2CC3(C(=O)N4CC[C@H](N)C(C)(C)C4)CC1CC(c1ccccc1)(C2)C3 ; COCC=C1C2CC3(C(=O)N[C@H]4CC[C@H](N)CC4)CC1CC(c1ccccc1)(C2)C3 ; COCC=C1C2CC3(C(=O)N4CC[C@H](N)C(C)(C)C4)CC1CC(c1ccccc1)(C2)C3 ; N#CCC1C2CC3(C(=O)N[C@H]4CC[C@H](N)CC4)CC1CC(c1ccccc1)(C2)C3 ; CC1(C)CN(C(=O)C23CC4CC(c5ccccc5)(CC(C2)C4CC#N)C3)CC[C@@H]1N ; N#CC=C1C2CC3(C(=O)N[C@H]4CC[C@H](N)CC4)CC1CC(c1ccccc1)(C2)C3]

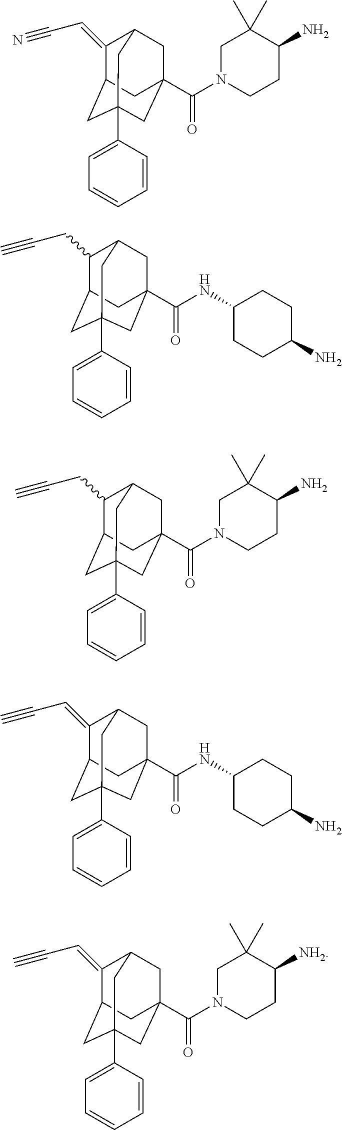

### Definitions

As used herein, the terms “comprising” and “including” are used in their open, non-limiting sense.

The terms “halo” and/or “halogen” refer to fluorine, chlorine, bromine or iodine.

The term “(C₁ to C₁₀) alkyl” refers to a saturated aliphatic hydrocarbon radical including straight chain and branched chain groups of 1 to 8 carbon atoms. Examples of (C₁ to C₁₀) alkyl groups include methyl, ethyl, propyl, 2-propyl, n-butyl, iso-butyl, tert-butyl, pentyl, and the like. The terms “Me” and “methyl,” as used herein, mean a —CH₃ group. The terms “Et” and “ethyl,” as used herein, mean a —C₂H₅ group.

The term “(C₂ to C₁₀) alkenyl”, as used herein, means an alkyl moiety comprising 2 to 10 carbons having at least one carbon-carbon double bond. The carbon-carbon double bond in such a group may be anywhere along the 2 to 10 carbon chain that will result in a stable compound. Such groups include both the E and Z isomers of said alkenyl moiety. Examples of such groups include, but are not limited to, ethene, propene, 1-butene, 2-butene, 1-pentene, 2-pentene, 1-hexene, 2-hexene, and 3-hexene. Examples of such groups include, but are not limited to, ethenyl, propenyl, butenyl, allyl, and pentenyl.

The term “allyl,” as used herein, means a —CH₂CH═CH₂ group.

As used herein, the term “(C₂ to C₁₀) alkynyl” means an alkyl moiety comprising from 2 to 8 carbon atoms and having at least one carbon-carbon triple bond. The carbon-carbon triple bond in such a group may be anywhere along the 2 to 10 carbon chain that will result in a stable compound. Examples of such groups include, but are not limited to, ethyne, propyne, 1-butyne, 2-butyne, 1-pentyne, 2-pentyne, 1-hexyne, 2-hexyne, and 3-hexyne.

The term “(C₁ to C₁₀) alkoxy”, as used herein, means an O-alkyl group wherein said alkyl group contains from 1 to 8 carbon atoms and is straight, branched, or cyclic. Examples of such groups include, but are not limited to, methoxy, ethoxy, n-propyloxy, iso-propyloxy, n-butoxy, iso-butoxy, tert-butoxy, cyclopentyloxy, and cyclohexyloxy.

The term “(C₆ to C₁₀) aryl”, as used herein, means a group derived from an aromatic hydrocarbon containing from 6 to 10 carbon atoms. Examples of such groups include, but are not limited to, phenyl or naphthyl. The terms “Ph” and “phenyl,” as used herein, mean a —C₆H₅ group. The term “benzyl,” as used herein, means a —CH₂C₆H₅ group.

The term “(C₆ to C₁₀) arylene” is art-regognized, and as used herein pertains to a bivalent moiety obtained by removing a hydrogen atom from a (C to C₁₀) aryl ring, as defined above.

“(C₂ to C₉) heteroaryl”, as used herein, means an aromatic heterocyclic group having a total of from 5 to 10 atoms in its ring, and containing from 2 to 9 carbon atoms and from one to four heteroatoms each independently selected from O, S and N, and with the proviso that the ring of said group does not contain two adjacent O atoms or two adjacent S atoms. The heterocyclic groups include benzo-fused ring systems. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The C₂ to C₉ heteroaryl groups may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached).

The term “(C₂ to C₁₀) heteroarylene” is art-recognized, and as used herein pertains to a bivalent moiety obtained by removing a hydrogen atom from a (C₆ to C₁₀) heteroaryl ring, as defined above.

The term “(C₂ to C₁₀) cycloheteroalkyl”, as used herein, means a non-aromatic, monocyclic, bicyclic, tricyclic, spirocyclic, or tetracyclic group having a total of from 4 to 13 atoms in its ring system, and containing from 2 to 10 carbon atoms and from 1 to 4 heteroatoms each independently selected from O, S and N, and with the proviso that the ring of said group does not contain two adjacent O atoms or two adjacent S atoms. Furthermore, such (C₂ to C₁₀) cycloheteroalkyl groups may contain an oxo substituent at any available atom that will result in a stable compound. For example, such a group may contain an oxo atom at an available carbon or nitrogen atom. Such a group may contain more than one oxo substituent if chemically feasible. In addition, it is to be understood that when such a (C₂ to C₁₀) cycloheteroalkyl group contains a sulfur atom, said sulfur atom may be oxidized with one or two oxygen atoms to afford either a sulfoxide or sulfone. An example of a 4 membered cycloheteroalkyl group is azetidinyl (derived from azetidine). An example of a 5 membered cycloheteroalkyl group is pyrrolidinyl. An example of a 6 membered cycloheteroalkyl group is piperidinyl. An example of a 9 membered cycloheteroalkyl group is indolinyl. An example of a 10 membered cycloheteroalkyl group is 4H-quinolizinyl. Further examples of such (C₂ to C₁₀) cycloheteroalkyl groups include, but are not limited to, tetrahydrofuranyl, dihydrofuranyl, tetrahydrothienyl, tetrahydropyranyl, dihydropyranyl, tetrahydroth iopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, quinolizinyl, 3-oxopiperazinyl, 4-methylpiperazinyl, 4-ethylpiperazinyl, and 1-oxo-2,8-diazaspiro[4.5]dec-8-yl. The (C₂ to C₁₀) heteroaryl groups may be C-attached or N-attached where such is possible. For instance, a group derived from piperazine may be piperazin-1-yl (N-attached) or piperazin-2-yl (C-attached).

The term “(C₂ to C₁₀) cycloheteroalkylene” is art-recognized, and as used herein pertains to a bidentate moiety obtained by removing a hydrogen atom from a (C₆ to C₁₀) cycloheteroalkyl ring, as defined above.

The term “(C₃ to C₁₀) cycloalkyl group” means a saturated, monocyclic, fused, spirocyclic, or polycyclic ring structure having a total of from 3 to 10 carbon 5 ring atoms. Examples of such groups include, but are not limited to, cyclopropyl, cyclobutyl, cyclopentyl, cyclopentenyl, cyclohexyl, cycloheptyl, and adamantyl.

The term “(C₃ to C₁₀) cycloalkylene” is art-recognized, and as used herein pertains to a bidentate moiety obtained by removing a hydrogen atom from a (C₃ to C₁₀) cycloalkyl ring, as defined above.

The term “spirocyclic” as used herein has its conventional meaning, that is, any compound containing two or more rings wherein two of the rings have one ring carbon in common. The rings of a spirocyclic compound, as herein defined, independently have 3 to 20 ring atoms. Preferably, they have 3 to 10 ring atoms. Non-limiting examples of a spirocyclic compound include spiro[3.3]heptane, spiro[3.4]octane, and spiro[4.5]decane.

The term “(C₅ to C) cycloalkenyl” means an unsaturated, monocyclic, fused, spirocyclic ring structures having a total of from 5 to 8 carbon ring atoms. Examples of such groups include, but not limited to, cyclopentenyl, cyclohexenyl.

The term “cyano” refers to a —C≡N group.

An “aldehyde” group refers to a carbonyl group, —C(O)R, where R is hydrogen.

An “alkoxy” group refers to both an —O-alkyl and an —O-cycloalkyl group, as defined herein.

An “alkoxycarbonyl” refers to a —C(O)OR.

An “alkylaminoalkyl” group refers to an -alkyl-NR-alkyl group.

An “alkylsulfonyl” group refer to a —SO₂ alkyl.

An “amino” group refers to an —NH₂ or an —NRR′ group.

An “aminoalkyl” group refers to an -alky-NRR′ group.

An “aminocarbonyl” refers to a —C(O)NRR′.

An “arylalkyl” group refers to -alkylaryl, where alkyl and aryl are defined herein.

An “aryloxy” group refers to both an —O-aryl and an —O-heteroaryl group, as defined herein.

An “aryloxycarbonyl” refers to —C(O)O-aryl.

An “arylsulfonyl” group refers to a —SO₂-aryl.

A “C-amido” group refers to a —C(O)NRR′ group.

A “carbonyl” group refers to a —C(O)R.

A “C-carboxyl” group refers to a —C(O)OR groups.

A “carboxylic acid” group refers to a C-carboxyl group in which R is hydrogen.

A “cyano” group refers to a —CN group.

A “dialkylaminoalkyl” group refers to an -(alkyl)N(alkyl)₂ group.

A “halo” or “halogen” group refers to fluorine, chlorine, bromine or iodine.

A “haloalkyl” group refers to an alkyl group substituted with one or more halogen atoms.

A “heteroalicycloxy” group refers to a heteroalicyclic-O group with heteroalicyclic as defined herein.

A “heteroaryloxyl” group refers to a heteroaryl-O— group with heteroaryl as defined herein.

A “hydroxy” group refers to an —OH group.

An “N-amido” group refers to a —R′C(O)NR group.

An “N-carbamyl” group refers to a —ROC(O)NR— group.

A “nitro” group refers to a —NO₂ group.

An “N-Sulfonamido” group refers to a —NR—S(O)₂R group.

An “N-thiocarbamyl” group refers to a ROC(S)NR′ group.

An “O-carbamyl” group refers to a —OC(O)NRR′ group.

An “O-carboxyl” group refers to a RC(O)O— group.

An “O-thiocarbamyl” group refers to a —OC(S)NRR′ group.

An “oxo” group refers to a carbonyl moiety such that alkyl substituted by oxo refers to a ketone group.

A “perfluoroalkyl group” refers to an alkyl group where all of the hydrogen atoms have been replaced with fluorine atoms.

A “phosphonyl” group refers to a —P(O)(OR)₂ group.

A “silyl” group refers to a —SiR₃ group.

An “S-sulfonamido” group refers to a —S(O)₂NR— group.

A “sulfinyl” group refers to a —S(O)R group.

A “sulfonyl” group refers to a —S(O)₂R group.

A “thiocarbonyl” group refers to a —C(═S)—R group.

A “trihalomethanecarbonyl” group refers to a Z₃CC(O)— group, where Z is halogen.

A “trihalomethanesulfonamido” group refers to a Z₃CS(O)₂NR— group, where Z is halogen.

A “trihalomethanesulfonyl” group refers to a Z₃CS(O)₂— group, where Z is halogen.

A “trihalomethyl” group refers to a —CZ₃ group.

A “C-carboxyl” group refers to a —C(O)OR groups.

The term “substituted,” means that the specified group or moiety bears one or more substituents.

The term “unsubstituted,” means that the specified group bears no substituents. The term “optionally substituted” means that the specified group is unsubstituted or substituted by one or more substituents. It is to be understood that in the compounds of the present invention when a group is said to be “unsubstituted,” or is “substituted” with fewer groups than would fill the valencies of all the atoms in the compound, the remaining valencies on such a group are filled by hydrogen. For example, if a C₆ aryl group, also called “phenyl” herein, is substituted with one additional substituent, one of ordinary skill in the art would understand that such a group has 4 open positions left on carbon atoms of the C₆ aryl ring (6 initial positions, minus one to which the remainder of the compound of the present invention is bonded, minus an additional substituent, to leave 4). In such cases, the remaining 4 carbon atoms are each bound to one hydrogen atom to fill their valencies. Similarly, if a C₆ aryl group in the present compounds is said to be “disubstituted,” one of ordinary skill in the art would understand it to mean that the C₆ aryl has 3 carbon atoms remaining that are unsubstituted. Those three unsubstituted carbon atoms are each bound to one hydrogen atom to fill their valencies.

The term “solvate,” is used to describe a molecular complex between compounds of the present invention and solvent molecules. Examples of solvates include, but are not limited to, compounds of the invention in combination with water, isopropanol, ethanol, methanol, dimethylsulfoxide (DMSO), ethyl acetate, acetic acid, ethanolamine, or mixtures thereof.

The term “hydrate” can be used when said solvent is water. It is specifically contemplated that in the present invention one solvent molecule can be associated with one molecule of the compounds of the present invention, such as a hydrate. Furthermore, it is specifically contemplated that in the present invention, more than one solvent molecule may be associated with one molecule of the compounds of the present invention, such as a dihydrate.

Additionally, it is specifically contemplated that in the present invention less than one solvent molecule may be associated with one molecule of the compounds of the present invention, such as a hemihydrate. Furthermore, solvates of the present invention are contemplated as solvates of compounds of the present invention that retain the biological effectiveness of the non-hydrate form of the compounds.

The term “pharmaceutically acceptable salt,” as used herein, means a salt of a compound of the present invention that retains the biological effectiveness of the free acids and bases of the specified derivative and that is not biologically or otherwise undesirable.

The term “pharmaceutically acceptable formulation”, as used herein, means a combination of a compound of the invention, or a salt or solvate thereof, and a carrier, diluent, and/or excipient(s) that are compatible with a compound of the present invention, and is not deleterious to the recipient thereof. Pharmaceutical formulations can be prepared by procedures known to those of ordinary skill in the art. For example, the compounds of the present invention can be formulated with common excipients, diluents, or carriers, and formed into tablets, capsules, and the like. Examples of excipients, diluents, and carriers that are suitable for such formulations include the following: fillers and extenders such as starch, sugars, mannitol, and silicic derivatives; binding agents such as carboxymethyl cellulose and other cellulose derivatives, alginates, gelatin, and polyvinyl pyrrolidone; moisturizing agents such as glycerol; disintegrating agents such as povidone, sodium starch glycolate, sodium carboxymethylcellulose, agar, calcium carbonate, and sodium bicarbonate; agents for retarding dissolution such as paraffin; resorption accelerators such as quaternary ammonium compounds; surface active agents such as cetyl alcohol, glycerol monostearate; adsorptive carriers such as kaolin and bentonite; and lubricants such as talc, calcium and magnesium stearate and solid polyethylene glycols. Final pharmaceutical forms may be pills, tablets, powders, lozenges, saches, cachets, or sterile packaged powders, and the like, depending on the type of excipient used. Additionally, it is specifically contemplated that pharmaceutically acceptable formulations of the present invention can contain more than one active ingredient. For example, such formulations may contain more than one compound according to the present invention.

The term “virus inhibiting amount” as used herein, refers to the amount of a compound of the present invention, or a salt or solvate thereof, required to inhibit the cell entry of an enveloped virus in vivo, such as in a mammal, or in vitro. The amount of such compounds required to cause such inhibition can be determined without undue experimentation using methods described herein and those known to those of ordinary skill in the art.

The terms “treat”, “treating”, and “treatment” with reference to enveloped virus infection, in mammals, particularly a human, include: (i) preventing the disease or condition from occurring in a subject which may be predisposed to the condition, such that the treatment constitutes prophylactic treatment for the pathologic condition; (ii) modulating or inhibiting the disease or condition, i.e., arresting its development; (iii) relieving the disease or condition, i.e., causing regression of the disease or condition; or (iv) relieving and/or alleviating the disease or condition or the symptoms resulting from the disease or condition.

The compositions are delivered in effective amounts. The term “effective amount” refers to the amount necessary or sufficient to realize a desired biologic effect and/or reduce the viral load. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is effective to treat the particular subject. In addition, based on testing, toxicity of the inhibitor is expected to be low. The effective amount for any particular application can vary depending on such factors as the disease or condition being treated, the particular inhibitor being administered, the size of the subject, or the severity of the disease or condition. One of ordinary skill in the art can empirically determine the effective amount of a particular inhibitor and/or other therapeutic agent without necessitating undue experimentation. It is preferred generally that a maximum dose be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug.

“Dose” and “dosage” are used interchangeably herein. For any compound described herein, the therapeutically effective amount can be initially determined from preliminary in vitro studies and/or animal models. A therapeutically effective dose can also be determined from human data for inhibitors that have been tested in humans and for compounds, which are known to exhibit similar pharmacological activities, such as other related active agents. The applied dose can be adjusted based on the relative bioavailability and potency of the administered compound. Adjusting the dose to achieve maximal efficacy based on the methods described above and other methods well-known in the art, is well within the capabilities of the ordinarily skilled artisan. In certain embodiments, the methods of the invention are useful for treating infection with enveloped viruses.

Unless indicated otherwise, all references herein to the inventive compounds include references to salts, solvates, and complexes thereof, including polymorphs, stereoisomers, tautomers, and isotopically labeled versions thereof. For example, compounds of the present invention can be pharmaceutically acceptable salts and/or pharmaceutically acceptable solvates.

The term “stereoisomers” refers to compounds that have identical chemical constitution, but differ with regard to the arrangement of their atoms or groups in space. In particular, the term “enantiomers” refers to two stereoisomers of a compound that are non-superimposable mirror images of one another.

A pure enantiomer can be contaminated with up to about 10% of the opposite enantiomer.

The terms “racemic” or “racemic mixture,” as used herein, refer to a 1:1 mixture of enantiomers of a particular compound. The term “diastereomers”, on the other hand, refers to the relationship between a pair of stereoisomers that comprise two or more asymmetric centers and are not mirror images of one another.

In accordance with a convention used in the art, the symbol is used in structural formulas herein to depict the bond that is the point of attachment of the moiety or substituent to the core or backbone structure. In accordance with another convention, in some structural formulae herein the carbon atoms and their bound hydrogen atoms are not explicitly depicted, e.g.,

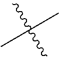

represents a methyl group,

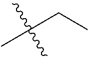 [SMILES: *CC]

represents an ethyl group,

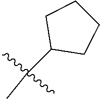 [SMILES: *C1CCCC1]

represents a cyclopentyl group, etc.

The compounds of the present invention may have asymmetric carbon atoms. The carbon-carbon bonds of the compounds of the present invention may be depicted herein using a solid line ( [symbol] ), a solid wedge ( [symbol] ), or a dotted wedge ( [symbol] ). The use of a solid line to depict bonds to asymmetric carbon atoms is meant to indicate that all possible stereoisomers (e.g. specific enantiomers, racemic mixtures, etc.) at that carbon atom are included. The use of either a solid or dotted wedge to depict bonds to asymmetric carbon atoms is meant to indicate that only the stereoisomer shown is meant to be included. It is possible that compounds of the invention may contain more than one asymmetric carbon atom. In those compounds, the use of a solid line to depict bonds to asymmetric carbon atoms is meant to indicate that all possible stereoisomers are meant to be included. For example, unless stated otherwise, it is intended that the compounds of the present invention can exist as enantiomers and diastereomers or as racemates and mixtures thereof. The use of a solid line to depict bonds to one or more asymmetric carbon atoms in a compound of the invention and the use of a solid or dotted wedge to depict bonds to other asymmetric carbon atoms in the same compound is meant to indicate that a mixture of diastereomers is present.

(“R”) unless otherwise defined, a substituent “R” may reside on any atom of the ring system, assuming replacement of a depicted, implied, or expressly defined hydrogen from one of the ring atoms, so long as a stable structure is formed.

Conventional techniques for the preparation/isolation of individual enantiomers include chiral synthesis from a suitable optically pure precursor or resolution of the racemate using, for example, chiral high performance liquid chromatography (HPLC). Alternatively, the racemate (or a racemic precursor) may be reacted with a suitable optically active compound, for example, an alcohol, or, in the case where the compound contains an acidic or basic moiety, an acid or base such as tartaric acid or 1-phenyl ethyl amine. The resulting diastereomeric mixture may be separated by chromatography and/or fractional crystallization and one or both of the diastereoisomers converted to the corresponding pure enantiomer(s) by means well known to one skilled in the art. Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture. Stereoisomeric conglomerates may be separated by conventional techniques known to those skilled in the art. See, e.g. “Stereochemistry of Organic Compounds” by E L Eliel (Wiley, New York, 1994), the disclosure of which is incorporated herein by reference in its entirety.

Where a compound of the invention contains an alkenyl or alkenylene group, geometric cis/trans (or Z/E) isomers are possible. Where the compound contains, for example, a keto or oxime group or an aromatic moiety, tautomeric isomerism (‘tautomerism’) can occur. Examples of tautomerism include keto and enol tautomers. A single compound may exhibit more than one type of isomerism. Included within the scope of the invention are all stereoisomers, geometric isomers and tautomeric forms of the inventive compounds, including compounds exhibiting more than one type of isomerism, and mixtures of one or more thereof. Cis/trans isomers may be separated by conventional techniques well known to those skilled in the art, for example, chromatography and fractional crystallization.

The compounds of the present invention may be administered as prodrugs. Thus certain derivatives of compounds of Structural Formulae I, II, III, IV, V, VI, VIa, VIb, VII, and VIII which may have little or no pharmacological activity themselves can, when administered to a mammal, be converted into a compound of Structural Formulae I, II, III, IV, V, VI, VIa, VIb, VII, and VIII having the desired activity, for example, by hydrolytic cleavage. Such derivatives are referred to as “prodrugs”. Prodrugs can, for example, be produced by replacing appropriate functionalities present in the compounds of Structural Formulae I, II, III, IV, V, VI, VIa, VIb, VII, and VIII with certain moieties known to those skilled in the art. See, e.g. “Pro-drugs as Novel Delivery Systems”, Vol. 14, ACS Symposium Series (T Higuchi and W Stella) and “Bioreversible Carriers in Drug Design”, Pergamon Press, 1987 (ed. E B Roche, American Pharmaceutical Association), the disclosures of which are incorporated herein by reference in their entireties. Some examples of such prodrugs include: an ester moiety in the place of a carboxylic acid functional group; an ether moiety or an amide moiety in place of an alcohol functional group; and an amide moiety in place of a primary or secondary amino functional group. Further examples of replacement groups are known to those of skill in the art. See, e.g. “Design of Prodrugs” by H Bundgaard (Elsevier, 1985), the disclosure of which is incorporated herein by reference in its entirety. It is also possible that certain compounds of Structural Formulae I, II, III, IV, V, VI, VIa, VIb, VII, and VIII may themselves act as prodrugs of other compounds of Structural Formulae I, II, III, IV, V, VI, VIa, VIb, VII, and VIII.

Salts of the present invention can be prepared according to methods known to those of skill in the art. Examples of salts include, but are not limited to, acetate, acrylate, benzenesulfonate, benzoate (such as chlorobenzoate, methylbenzoate, dinitrobenzoate, hydroxybenzoate, and methoxybenzoate), bicarbonate, bisulfate, bisulfite, bitartrate, borate, bromide, butyne-1,4-dioate, calcium edetate, camsylate, carbonate, chloride, caproate, caprylate, clavulanate, citrate, decanoate, dihydrochloride, dihydrogenphosphate, edetate, edislyate, estolate, esylate, ethylsuccinate, formate, fumarate, gluceptate, gluconate, glutamate, glycollate, glycollylarsanilate, heptanoate, hexyne-1,6-dioate, hexylresorcinate, hydrabamine, hydrobromide, hydrochloride, γ-hydroxybutyrate, iodide, isobutyrate, isothionate, lactate, lactobionate, laurate, malate, maleate, malonate, mandelate, mesylate, metaphosphate, methanesulfonate, methylsulfate, monohydrogenphosphate, mucate, napsylate, naphthalene-1-sulfonate, naphthalene-2-sulfonate, nitrate, oleate, oxalate, pamoate (embonate), palmitate, pantothenate, phenylacetates, phenylbutyrate, phenylpropionate, phthalate, phospate/diphosphate, polygalacturonate, propanesulfonate, propionate, propiolate, pyrophosphate, pyrosulfate, salicylate, stearate, subacetate, suberate, succinate, sulfate, sulfonate, sulfite, tannate, tartrate, teoclate, tosylate, triethiodode, and valerate salts.

The compounds of the present invention that are basic in nature are capable of forming a wide variety of different salts with various inorganic and organic acids. Although such salts must be pharmaceutically acceptable for administration to animals or humans, it is often desirable in practice to initially isolate the compound of the present invention from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention can be prepared by treating the base compound with a substantially equivalent amount of the selected mineral or organic acid in an aqueous solvent medium or in a suitable organic solvent, such as methanol or ethanol. Upon evaporation of the solvent, the desired solid salt is obtained. The desired acid salt can also be precipitated from a solution of the free base in an organic solvent by adding an appropriate mineral or organic acid to the solution.

Those compounds of the present invention that are acidic in nature are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the acidic compounds of the present invention. Such non-toxic base salts include those derived from such pharmacologically acceptable cations as sodium, potassium, calcium, and magnesium, etc. These salts can be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

If the inventive compound is a base, the desired salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the inventive compound is an acid, the desired salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

In the case of agents that are solids, it is understood by those skilled in the art that the inventive compounds, agents and salts may exist in different crystal or polymorphic forms, all of which are intended to be within the scope of the present invention and specified formulas.

The invention also includes isotopically-labeled compounds of the invention, wherein one or more atoms is replaced by an atom having the same atomic number, but an atomic mass or mass number different from the atomic mass or mass number usually found in nature. Examples of isotopes suitable for inclusion in the compounds of the invention include isotopes of hydrogen, such as ²H and ³H, carbon, such as 11, ¹³C and ¹⁴C, chlorine, such as ³⁶Cl, fluorine, such as ¹⁸F, iodine, such as ¹²³I and 1251, nitrogen, such as ¹³N and ¹⁵N, oxygen, such as ¹⁵O, ¹⁷O and ¹⁸O, phosphorus, such as ³²P, and sulfur, such as ³⁵S.

Certain isotopically-labeled compounds of the invention, for example, those incorporating a radioactive isotope, are useful in drug and/or substrate tissue distribution studies. The radioactive isotopes tritium, ³H, and carbon-14, ¹⁴C, are particularly useful for this purpose in view of their ease of incorporation and ready means of detection. Substitution with heavier isotopes such as deuterium, ²H, may afford certain therapeutic advantages resulting from greater metabolic stability, for example, ²H increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of the invention can generally be prepared by conventional techniques known to those skilled in the art or by processes analogous to those described herein, using an appropriate isotopically-labeled reagent in place of the non-labeled reagent otherwise employed.

The compounds of the present invention may be formulated into pharmaceutical compositions as described below in any pharmaceutical form recognizable to the skilled artisan as being suitable. Pharmaceutical compositions of the invention comprise a therapeutically effective amount of at least one compound of the present invention and an inert, pharmaceutically acceptable carrier or diluent.

To treat or prevent diseases or conditions mediated in part or whole by enveloped virus infection, a pharmaceutical composition of the invention is administered in a suitable formulation prepared by combining a therapeutically effective amount (i.e., an enveloped virus GP- or host cell partner-modulating, regulating, or inhibiting amount effective to achieve therapeutic efficacy) of at least one compound of the present invention (as an active ingredient) with one or more pharmaceutically suitable carriers, which may be selected, for example, from diluents, excipients and auxiliaries that facilitate processing of the active compounds into the final pharmaceutical preparations.

The pharmaceutical carriers employed may be either solid or liquid. Exemplary solid carriers are lactose, sucrose, talc, gelatin, agar, pectin, acacia, magnesium stearate, stearic acid and the like. Exemplary liquid carriers are syrup, peanut oil, olive oil, water and the like. Similarly, the inventive compositions may include time-delay or time-release material known in the art, such as glyceryl monostearate or glyceryl distearate alone or with a wax, ethylcellulose, hydroxypropylmethylcellulose, methylmethacrylate or the like. Further additives or excipients may be added to achieve the desired formulation properties. For example, a bioavailability enhancer, such as Labrasol, Gelucire or the like, or formulator, such as CMC (carboxy-methylcellulose), PG (propyleneglycol), or PEG (polyethyleneglycol), may be added. Gelucire®, a semi-solid vehicle that protects active ingredients from light, moisture and oxidation, may be added, e.g., when preparing a capsule formulation.

If a solid carrier is used, the preparation can be tableted, placed in a hard gelatin capsule in powder or pellet form, or formed into a troche or lozenge. The amount of solid carrier may vary, but generally, will be from about 25 mg to about 1 g. If a liquid carrier is used, the preparation may be in the form of syrup, emulsion, soft gelatin capsule, sterile injectable solution or suspension in an ampoule or vial or non-aqueous liquid suspension. If a semi-solid carrier is used, the preparation may be in the form of hard and soft gelatin capsule formulations. The inventive compositions are prepared in unit-dosage form appropriate for the mode of administration, e.g. parenteral or oral administration.

To obtain a stable water-soluble dose form, a salt of a compound of the present invention may be dissolved in an aqueous solution of an organic or inorganic acid, such as a 0.3 M solution of succinic acid or citric acid. If a soluble salt form is not available, the agent may be dissolved in a suitable co-solvent or combinations of co-solvents. Examples of suitable co-solvents include alcohol, propylene glycol, polyethylene glycol 300, polysorbate 80, glycerin and the like in concentrations ranging from 0 to 60% of the total volume. In an exemplary embodiment, a compound of the present invention is dissolved in DMSO and diluted with water. The composition may also be in the form of a solution of a salt form of the active ingredient in an appropriate aqueous vehicle such as water or isotonic saline or dextrose solution.

Proper formulation is dependent upon the route of administration selected. For injection, the agents of the compounds of the present invention may be formulated into aqueous solutions, preferably in physiologically compatible buffers such as Hanks solution, Ringer's solution, or physiological saline buffer.

For transmucosal administration, penetrants appropriate to the barrier to be permeated are used in the formulation. Such penetrants are generally known in the art.

For oral administration, the compounds can be formulated by combining the active compounds with pharmaceutically acceptable carriers known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethylcellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures.

Dyestuffs or pigments may be added to the tablets or dragee coatings for identification or to characterize different combinations of active agents.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration intranasally or by inhalation, the compounds for use according to the present invention may be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount.

Capsules and cartridges of gelatin for use in an inhaler or insufflator and the like may be formulated containing a powder mix of the compound and a suitable powder base such as lactose or starch.

The compounds may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Alternatively, the active ingredient may be in powder form for constitution with a suitable vehicle, e.g. sterile pyrogen-free water, before use.

In addition to the formulations described above, the compounds of the present invention may also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt. A pharmaceutical carrier for hydrophobic compounds is a cosolvent system comprising benzyl alcohol, a non-polar surfactant, a water-miscible organic polymer, and an aqueous phase. The co-solvent system may be a VPD co-solvent system. VPD is a solution of 3% w/v benzyl alcohol, 8% w/v of the non-polar surfactant polysorbate 80, and 65% w/v polyethylene glycol 300, made up to volume in absolute ethanol. The VPD co-solvent system (VPD: 5W) contains VPD diluted 1:1 with a 5% dextrose in water solution. This co-solvent system dissolves hydrophobic compounds well, and itself produces low toxicity upon systemic administration. The proportions of a cosolvent system may be suitably varied without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity due to the toxic nature of DMSO. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. These carriers and excipients may provide marked improvement in the bioavailability of poorly soluble drugs. Examples of such carriers or excipients include calcium carbonate, calcium phosphate, sugars, starches, cellulose derivatives, gelatin, and polymers such as polyethylene glycols. Furthermore, additives or excipients such as Gelucire®, Capryol®, Labrafil®, Labrasol®, Lauroglycol®, Plurol®, Peceol®, Transcutol® and the like may be used.

Further, the pharmaceutical composition may be incorporated into a skin patch for delivery of the drug directly onto the skin.

It will be appreciated that the actual dosages of the agents of this invention will vary according to the particular agent being used, the particular composition formulated, the mode of administration, and the particular site, host, and disease being treated. Those skilled in the art using conventional dosage determination tests in view of the experimental data for a given compound may ascertain optimal dosages for a given set of conditions. For oral administration, an exemplary daily dose generally employed will be from about 0.001 to about 1000 mg/kg of body weight, with courses of treatment repeated at appropriate intervals.

Furthermore, the pharmaceutically acceptable formulations of the present invention may contain a compound of the present invention, or a salt or solvate thereof, in an amount of about 10 mg to about 2000 mg, or from about 10 mg to about 1500 mg, or from about 10 mg to about 1000 mg, or from about 10 mg to about 750 mg, or from about 10 mg to about 500 mg, or from about 25 mg to about 500 mg, or from about 50 to about 500 mg, or from about 100 mg to about 500 mg.

Additionally, the pharmaceutically acceptable formulations of the present invention may contain a compound of the present invention, or a salt or solvate thereof, in an amount from about 0.5 w/w % to about 95 w/w %, or from about 1 w/w % to about 95 w/w %, or from about 1 w/w % to about 75 w/w %, or from about 5 w/w % to about 75 w/w %, or from about 10 w/w % to about 75 w/w %, or from about 10 w/w % to about 50 w/w %.

The compounds of the present invention, or salts or solvates thereof, may be administered to a mammal, such as a human, suffering from a condition or disease mediated by an enveloped virus, either alone or as part of a pharmaceutically acceptable formulation, once a day, twice a day, three times a day, four times a day, or even more frequently.

The compounds of the present invention, or salts or solvates thereof, may be administered to humans or mammals suffering from a condition or disease mediated by a filovirus, arenavirus, or other enveloped virus in combination with at least one other agent used for treatment, alone or as part of a pharmaceutically acceptable formulation, once a day, twice a day, three times a day, four times a day, or even more frequently.

Those of ordinary skill in the art will understand that with respect to the compounds of the present invention, the particular pharmaceutical formulation, the dosage, and the number of doses given per day to humans or mammals requiring such treatment, are all choices within the knowledge of one of ordinary skill in the art and can be determined without undue experimentation.

### Combination Therapy

Compounds of Structural Formulae I, II, III, IV, V, VI, VIa, VIb, VII, and VIII of the invention may be combined with other therapeutic agents. The inhibitor and other therapeutic agent may be administered simultaneously or sequentially. When the other therapeutic agents are administered simultaneously, they can be administered in the same or separate formulations, but are administered at the same time. The other therapeutic agents are administered sequentially with one another and with the inhibitors, when the administration of the other therapeutic agents and the inhibitors is temporally separated. The separation in time between the administration of these compounds may be a matter of minutes or it may be longer. Other therapeutic agents include but are not limited to anti-viral vaccines and anti-viral agents. In some instanses the inhibitors are administered with multiple therapeutic agents, i.e., 2, 3, 4 or even more different anti-viral agents.

An anti-viral vaccine is a formulation composed of one or more viral antigens and one or more adjuvants. The viral antigens include proteins or fragments thereof as well as whole killed virus. Adjuvants are well known to those of skill in the art.

Antiviral agents are compounds, which prevent infection of cells by viruses or replication of the virus within the cell. There are many fewer antiviral drugs than antibacterial drugs because viruses are more dependent on host cell factors than bacteria. There are several stages within the process of viral infection, which can be blocked or inhibited by antiviral agents. These stages include, attachment of the virus to the host cell (immunoglobulin or binding peptides), membrane penetration inhibitors, e.g. T-20, uncoating of the virus (e.g. amantadine), synthesis or translation of viral mRNA (e.g. interferon), replication of viral RNA or DNA (e.g. nucleotide analogues), maturation of new virus proteins (e.g. protease inhibitors), and budding and release of the virus.

Nucleotide analogues are synthetic compounds which are similar to nucleotides, but which have an incomplete or abnormal deoxyribose or ribose group. Once the nucleotide analogues are in the cell, they are phosphorylated, producing the triphosphate formed which competes with normal nucleotides for incorporation into the viral DNA or RNA. Once the triphosphate form of the nucleotide analogue is incorporated into the growing nucleic acid chain, it causes irreversible association with the viral polymerase and thus chain termination. Nucleotide analogues include, but are not limited to, acyclovir (used for the treatment of herpes simplex virus and varicella-zoster virus), gancyclovir (useful for the treatment of cytomegalovirus), idoxuridine, ribavirin (useful for the treatment of respiratory syncitial virus), dideoxyinosine, dideoxycytidine, zidovudine (azidothymidine), imiquimod, resimiquimod, favipiravir, BCX4430, and GS-5374 or their analogues.

The interferons are cytokines which are secreted by virus-infected cells as well as immune cells. The interferons function by binding to specific receptors on cells adjacent to the infected cells, causing the change in the cell which protects it from infection by the virus. α- and β-interferon also induce the expression of Class I and Class II MHC molecules on the surface of infected cells, resulting in increased antigen presentation for host immune cell recognition. α- and β-interferons are available as recombinant proteins and have been used for the treatment of chronic hepatitis B and C infection. At the dosages that are effective for anti-viral therapy, interferons may have severe side effects such as fever, malaise and weight loss.

Anti-viral agents, which may be useful in combination with Structural Formulae I, II, III, IV, V, VI, VIa, VIb, VII, and VIII of the invention, include but are not limited to immunoglobulins, amantadine, interferons, nucleotide analogues, small interfering RNAs (siRNAs) and other protease inhibitors (other than the papain-like cysteine protease inhibitors—although combinations of papain-like cysteine protease inhibitors are also useful). Specific examples of anti-viral agents include but are not limited to Acemannan; Acyclovir; Acyclovir Sodium; Adefovir; Alovudine; Alvircept Sudotox; Amantadine Hydrochloride; Aranotin; Arildone; Atevirdine Mesylate; AVI-7537: Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Favipiravir; Fiacitabine; Fialuridine; Fosarilate; Fosfonet; Fosfonet Sodium; Ganciclovir; Ganciclovir Sodium; Idoxuridine; Kethoxal; Lamivudinc; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; TKM Ebola; Triazavirin; Trifluridine; Valacyclovir Hydrochloride; Vidarabine; Vidarabine Phosphate; Vidarabine Sodium Phosphate; Viroxime; Zalcitabine; Zidovudine; Zinviroxime; and ZMapp.

Immunoglobulin therapy is used for the prevention of viral infection. Immunoglobulin therapy for viral infections is different than bacterial infections, because rather than being antigen-specific, the immunoglobulin therapy functions by binding to extracellular virions and preventing them from attaching to and entering cells which are susceptible to the viral infection. The therapy is useful for the prevention of viral infection for the period of time that the antibodies are present in the host. In general, there are two types of immunoglobulin therapies, normal immunoglobulin therapy and hyper-immunoglobulin therapy. Normal immune globulin therapy utilizes an antibody product which is prepared from the serum of normal blood donors and pooled. This pooled product contains low titers of antibody to a wide range of human viruses, such as hepatitis A, parvovirus, enterovirus (especially in neonates). Hyper-immune globulin therapy utilizes antibodies which are prepared from the serum of individuals who have high titers of an antibody to a particular virus. Those antibodies are then used against a specific virus. Another type of immunoglobulin therapy is active immunization. This involves the administration of antibodies or antibody fragments to viral surface proteins.

In the following Preparations and Examples, “Ac” means acetyl, “Me” means methyl, “Et” means ethyl, “Ph” means phenyl, “Py” means pyridine, “BOC”, “Boc” or “boc” means N-tert-butoxycarbonyl, “Ns” means 2-Nitrophenylsulfonyl, “CMMP” means (cyanomethylene) trimethyl phosphorane”, DCM” (CH₂Cl₂) means dichloromethane or methylene chloride, “DCE” means dichloroethane or ethylene chloride, “DIAD” means diisopropylazadicarboxylate, “DIPEA” or “DIEA” means diisopropyl ethyl amine, “DMA” means N,N-dimethylacetamide, “DMAP” means 4-dimethylaminopyridine, “DME” means 1,2-dimethoxyethane, “DMF” means N,N-dimethyl formamide, “DMSO” means dimethylsulfoxide, “DPPA” means diphenylphosphorylazide, “DPPP” means 1,3-bis(diphenylphosphino)propane, “EDCl” means 3-(ethyliminomethyleneamino)-N,N-dimethylpropan-1-amine, “EtOAc” means ethyl acetate, “HATU” means 1-[Bis(dimethylamino)methylene]-1H-1,2,3-triazolo[4,5-b]pyridinium 3-oxid hexafluorophosphate, “HOAt” means 1-hydroxy-7 azabenzotriazole, “HOAc” means acetic acid, “IPA” means isopropyl alcohol, “LDA” means lithium diisopropylamide, “NMP” means 1-methyl 2-pyrrolidinone, “TEA” means triethyl amine, “TFA” means trifluoroacetic acid, “TOSMIC” means toluenesulfonylmethyl isocyanide, “MgSO₄” means magnesium sulphate, “NaHMDS” or “NHMDS” means sodium hexamethyldisilazide, “Na₂SO₄” means sodium sulphate, “MeOH” means methanol, “Et20” means diethyl ether, “EtOH” means ethanol, “H₂O” means water, “HCl” means hydrochloric acid, “POCl₃” means phosphorus oxychloride, “SOCl₂” means thionylchloride, “K₂CO₃” means potassium carbonate, “THF” means tetrahydrofuran, “DBU” means 1,8-diazabicyclo[5.4.0]undec-7-ene, “LAH” means lithium aluminium hydride, “LiHMDS” or “LHMDS” means lithium hexamethyldisilazide, “TBABr” means tetra butyl ammonium bromide, “TBME” or “MTBE” means tert-butyl methyl ether, “TMS” means trimethylsilyl, “PMHS” means polymethylhydrosiloxane, “MCPBA” means 3-chloroperoxy benzoic acid, “N” means Normal, “M” means molar, “mL” means millilitre, “mmol” means millimoles, “pmol” means micromoles, “eq.” means equivalent, “° C.” means degrees Celsius, “Pa” means pascals, “Xanthphos” means 4,5-bis(diphenylphosphino)-9,9-dimethylxanthene, “r.t.” means room temperature.

### Methods of Preparation.

Compounds of the present invention may be prepared using the reaction routes and synthetic schemes described below, employing the techniques available in the art using starting materials that are readily available. The preparation of certain embodiments of the present invention is described in detail in the following examples, but those of ordinary skill in the art will recognize that the preparations described may be readily adapted to prepare other embodiments of the present invention. For example, the synthesis of non-exemplified compounds according to the invention may be performed by modifications apparent to those skilled in the art, e.g. by appropriately protecting interfering groups, by changing to other suitable reagents known in the art, or by making routine modifications of reaction conditions. Alternatively, other reactions referred to herein or known in the art will be recognized as having adaptability for preparing other compounds of the invention.

In one general synthetic process, compounds of the Structural Formula I wherein Y is CH₂, X is

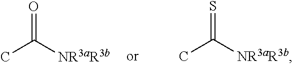

and Q is CH₂ or CR²³R²⁴, represented by Formulae I-a and I-b can be prepared according to Scheme 1. Carboxylic acid 1-1 can be reacted with amine NHR^{3a}R^{3b} in the presence of a coupling reagent such as EDCl or HATU and a base such as DIEA or triethylamine in a solvent such as DMF or dichloroethane to provide the desired product of Formula I-a. Alternatively, carboxylic acid 1-1 can react with SOCl₂ to form acid chloride 1-2 which can react with amine NHR^{3a}R^{3b} in presence of a base such as DIEA or triethylamine in a solvent such as DMF or dichloroethane to form the desired product of Formula I-a. This amide can react with Lawesson's reagent in a solvent such as tetrahydrofuran to form the desired product of Structural Formula I-b.

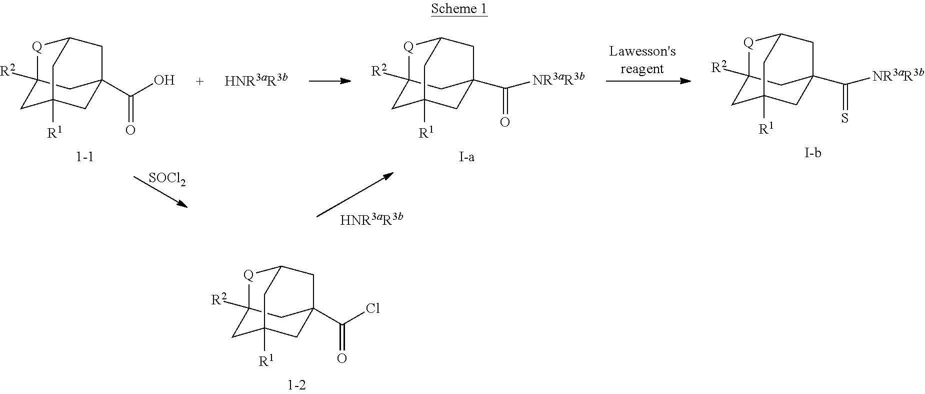

In another general synthetic process, compounds of the Structural Formula I wherein X is CH, Y is

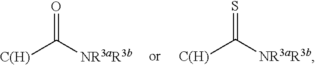

and Q is CH₂, represented by Formulae I-c and I-d can be prepared according to Scheme 2 by reacting carboxylic acid 2-1 with amine NHR^{3a}R^{3b} in the presence of a coupling reagent such as EDCl or HATU and a base such as DIEA or triethylamine in a solvent such as DMF or dichloroethane to provide the desired product of Formula I-c. Alternatively, carboxylic acid 2-1 can react with SOCl₂ to form acid chloride 2-2 which can react with amine NHR^{3a}R^{3b} in presence of a base such as DIEA or triethylamine in a solvent such as DMF or dichloroethane to form the desired product of Formula I-c. This amide can react with Lawesson's reagent in a solvent such as tetrahydrofuran to form the desired compound of Structural Formula I-d.

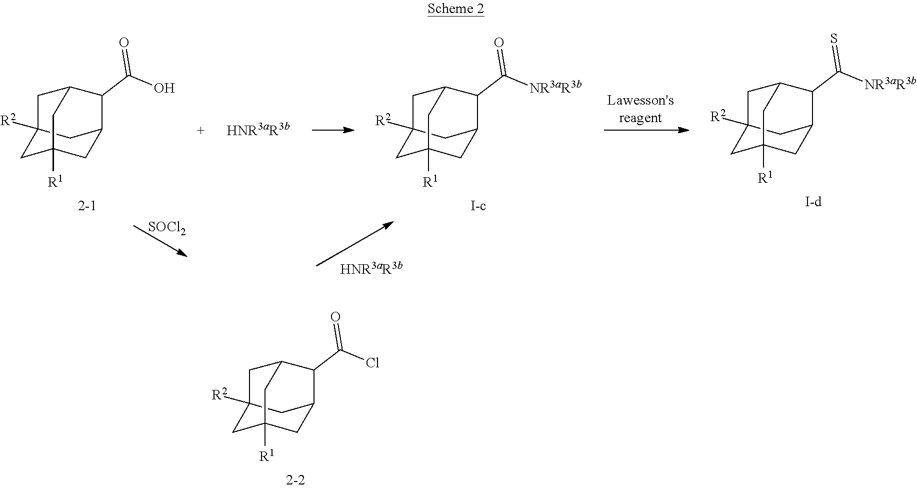

In another general synthetic process, compounds of the Structural Formula II wherein W is O or S, represented by Formulae II-a and II-b can be prepared according to Scheme 3. Enantiomerically pure carboxylic acid 3-1 can be reacted with amine NHR^{3a}R^{3b} in the presence of a coupling reagent such as EDCl or HATU and a base such as DIEA or triethylamine in a solvent such as DMF or dichloroethane to provide the desired product of Formula II-a. Alternatively, enantiomerically pure carboxylic acid 3-1 can react with SOCl₂ to form acid chloride 3-2 which can react with amine NHR^{3a}R^{3b} in presence of a base such as DIEA or triethylamine in a solvent such as DMF or dichloroethane to form the desired product of Formula II-a. This amide can react with Lawesson's reagent in a solvent such as tetrahydrofuran to form the desired product of Structural Formula II-b.

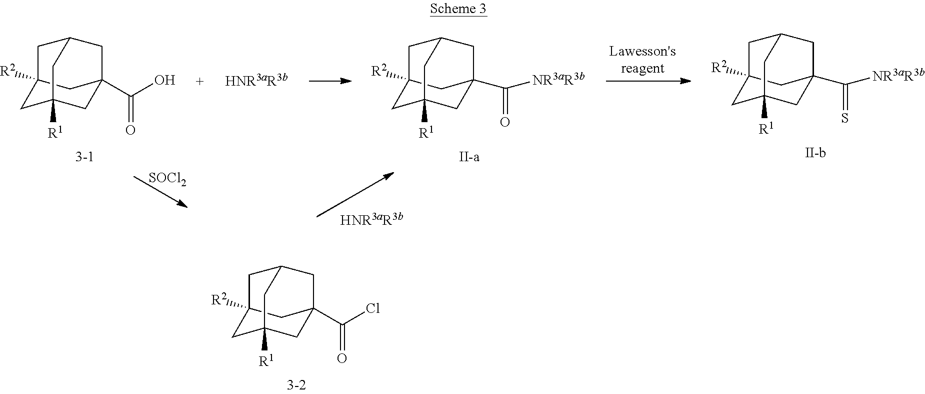

In another general synthetic process, compounds of the Structural Formula III wherein W is O or S, represented by Formulae III-a and III-b can be prepared according to Scheme 4. Enantiomerically pure carboxylic acid 4-1 can be reacted with amine NHR^{3a}R^{3b} in the presence of a coupling reagent such as EDCl or HATU and a base such as DIEA or triethylamine in a solvent such as DMF or dichloroethane to provide the desired product of Formula III-a. Alternatively, enantiomerically pure carboxylic acid 4-1 can react with SOCl₂ to form acid chloride 4-2 which can react with amine NHR^{3a}R^{3b} in presence of a base such as DIEA or triethylamine in a solvent such as DMF or dichloroethane to form the desired product of Formula III-a. This amide can react with Lawesson's reagent in a solvent such as tetrahydrofuran to form the desired product of Structural Formula III-b.

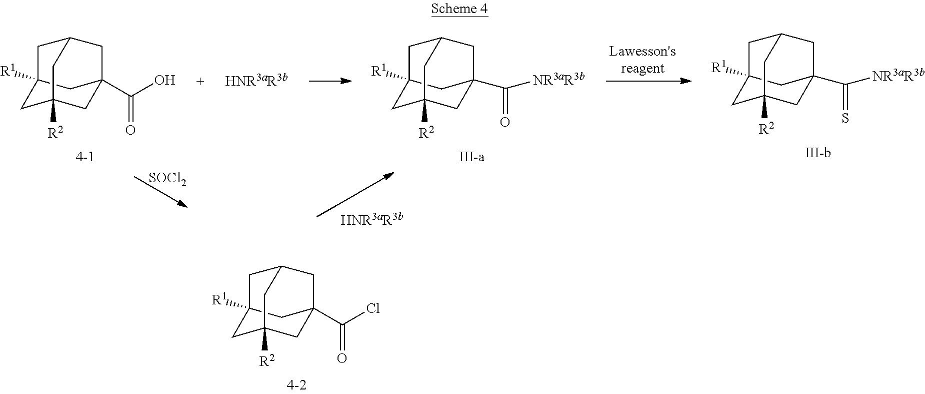

In another general synthetic process, compounds of the Structural Formula IV wherein W is O or S, represented by Formulae IV-a and IV-b can be prepared according to Scheme 5 by reacting carboxylic acid 5-1 with amine NHR^{3a}R^{3b} in the presence of a coupling reagent such as EDCl or HATU and a base such as DIEA or triethylamine in a solvent such as DMF or dichloroethane to provide the desired compound of Structural Formula IV-a. Alternatively, carboxylic acid 5-1 can react with SOCl₂ to form acid chloride 5-2 which can react with amine NHR^{3a}R^{3b} in presence of a base such as DIEA or triethylamine in a solvent such as DMF or dichloroethane to form the desired compound of Structural Formula IV-a. This amide can react with Lawesson's reagent in a solvent such as tetrahydrofuran to form the desired compound of Structural Formula IV-b.

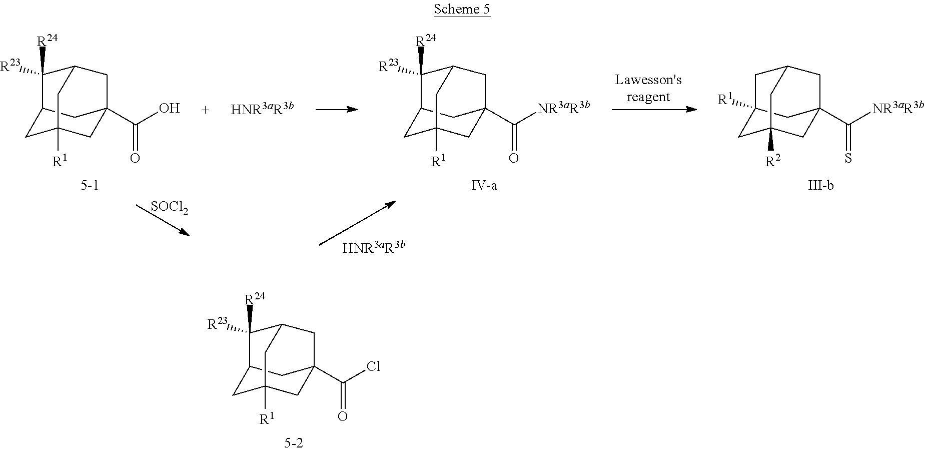

In another general synthetic process, compounds of the Structural Formula V wherein W is O or S, represented by Formulae V-a and V-b can be prepared according to Scheme 6. Carboxylic acid 6-1 can be reacted with amine NHR^{3a}R^{3b} in the presence of a coupling reagent such as EDCl or HATU and a base such as DIEA or triethylamine in a solvent such as DMF or dichloroethane to provide the desired product of Formula V-a. Alternatively, carboxylic acid 6-1 can react with SOCl₂ to form acid chloride 6-2 which can react with amine NHR^{3a}R^{3b} in presence of a base such as DIEA or triethylamine in a solvent such as DMF or dichloroethane to form the desired product of Formula V-a. This amide can react with Lawesson's reagent in a solvent such as tetrahydrofuran to form the desired product of Structural Formula V-b.

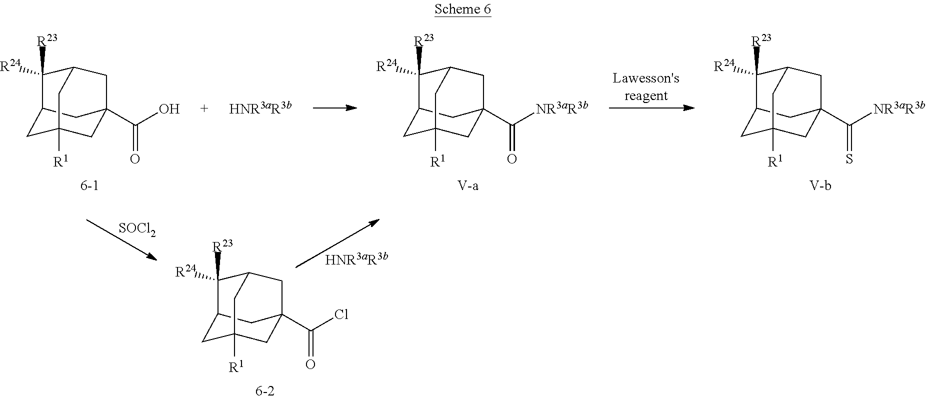

In another general synthetic process, compounds of the Structural Formula VI wherein W is O or S, represented by Formulae VI-a and VI-b can be prepared according to Scheme 7. Carboxylic acid 7-1 can be reacted with amine NHR^{3a}R^{3b} in the presence of a coupling reagent such as EDCl or HATU and a base such as DIEA or triethylamine in a solvent such as DMF or dichloroethane to provide the desired product of Formula VI-a. Alternatively, carboxylic acid 7-1 can react with SOCl₂ to form acid chloride 7-2 which can react with amine NHR^{3a}R^{3b} in presence of a base such as DIEA or triethylamine in a solvent such as DMF or dichloroethane to form the desired product of Formula VI-a. This amide can react with Lawesson's reagent in a solvent such as tetrahydrofuran to form the desired product of Structural Formula VI-b.

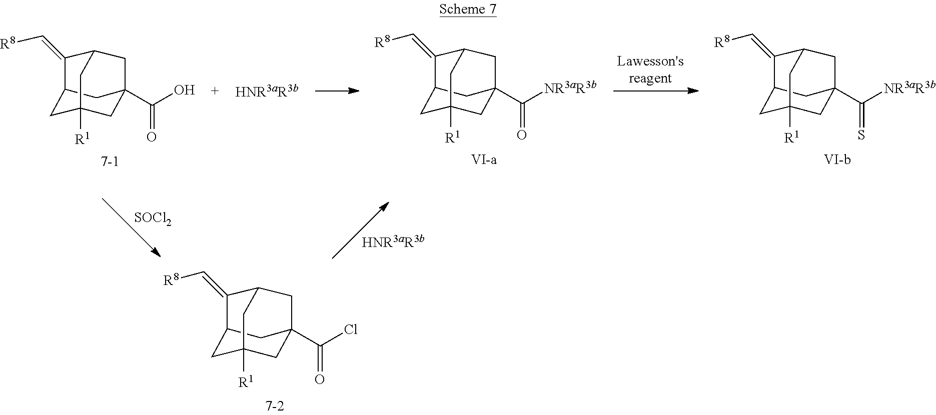

In another general synthetic process, compounds of the Structural Formula Via wherein W is O or S, represented by Formulae VIa-a and VIa-b can be prepared according to Scheme 8. Enantiomerically pure carboxylic acid 8-1 can be reacted with amine NHR^{3a}R^{3b} in the presence of a coupling reagent such as EDCl or HATU and a base such as DIEA or triethylamine in a solvent such as DMF or dichloroethane to provide the desired product of Formula VIa-a. Alternatively, enantiomerically pure carboxylic acid 8-1 can react with SOCl₂ to form acid chloride 8-2 which can react with amine NHR^{3a}R^{3b} in presence of a base such as DIEA or triethylamine in a solvent such as DMF or dichloroethane to form the desired product of Formula Via-a. This amide can react with Lawesson's reagent in a solvent such as tetrahydrofuran to form the desired product of Structural Formula Via-b.

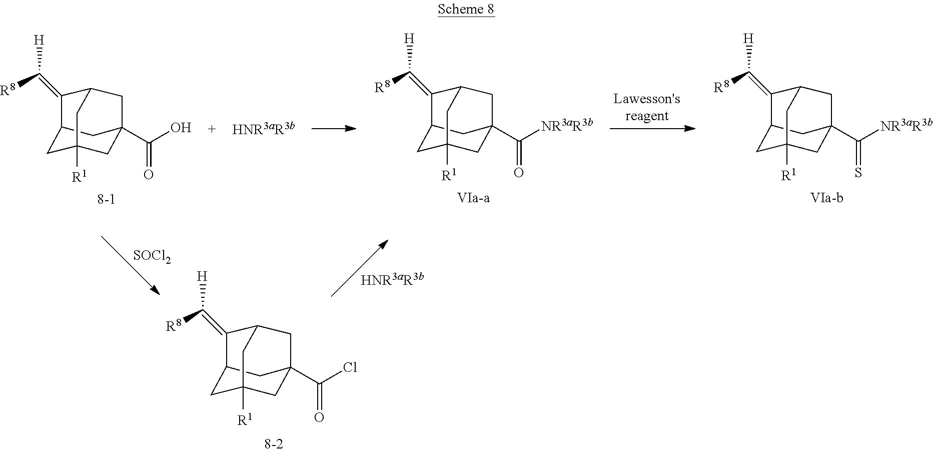

In another general synthetic process, compounds of the Structural Formula VIb wherein W is O or S, represented by Formulae VIb-a and VIb-b can be prepared according to Scheme 9. Enantiomerically pure carboxylic acid 9-1 can be reacted with amine NHR^{3a}R^{3b} in the presence of a coupling reagent such as EDCl or HATU and a base such as DIEA or triethylamine in a solvent such as DMF or dichloroethane to provide the desired product of Formula VIb-a. Alternatively, enantiomerically pure carboxylic acid 9-1 can react with SOCl₂ to form acid chloride 9-2 which can react with amine NHR^{3a}R^{3b} in presence of a base such as DIEA or triethylamine in a solvent such as DMF or dichloroethane to form the desired product of Formula VIb-a. This amide can react with Lawesson's reagent in a solvent such as tetrahydrofuran to form the desired product of Structural Formula VIb-b.

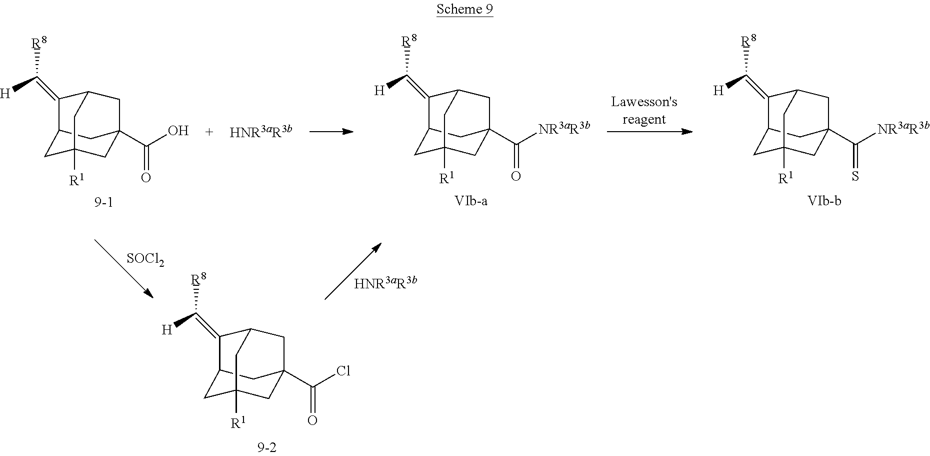

In another general synthetic process, compounds of the Structural Formula VII wherein W is O or S, represented by Formulae VII-a and VII-b can be prepared according to Scheme 10. Carboxylic acid 10-1 can be reacted with amine NHR^{3a}R^{3b} in the presence of a coupling reagent such as EDCl or HATU and a base such as DIEA or triethylamine in a solvent such as DMF or dichloroethane to provide the desired product of Formula VII-a. Alternatively, carboxylic acid 10-1 can react with SOCl₂ to form acid chloride 10-2 which can react with amine NHR^{3a}R^{3b} in presence of a base such as DIEA or triethylamine in a solvent such as DMF or dichloroethane to form the desired product of Formula VII-a. This amide can react with Lawesson's reagent in a solvent such as tetrahydrofuran to form the desired product of Structural Formula VII-b.

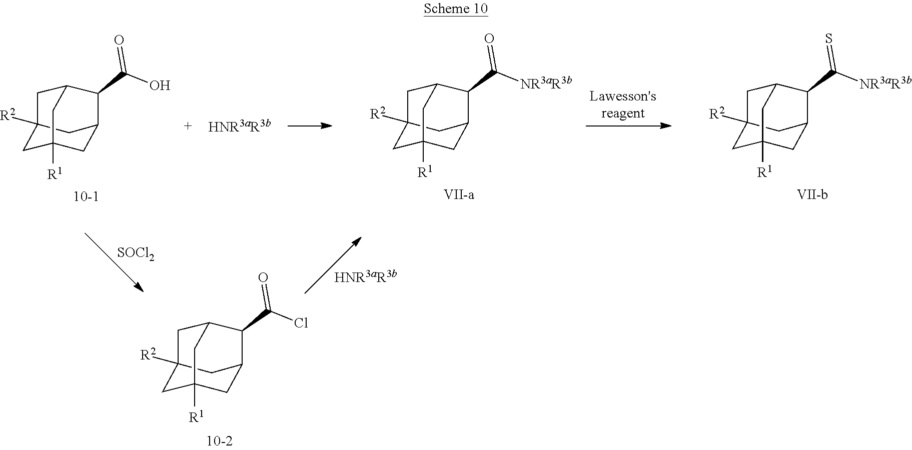

In another general synthetic process, compounds of the Structural Formula VIII wherein W is O or S, represented by Formulae VIII-a and VIII-b can be prepared according to Scheme 11. Carboxylic acid 11-1 can be reacted with amine NHR^{3a}R^{3b} in the presence of a coupling reagent such as EDCl or HATU and a base such as DIEA or triethylamine in a solvent such as DMF or dichloroethane to provide the desired product of Formula VIII-a. Alternatively, carboxylic acid 11-1 can react with SOCl₂ to form acid chloride 11-2 which can react with amine NHR^{3a}R^{3b} in presence of a base such as DIEA or triethylamine in a solvent such as DMF or dichloroethane to form the desired product of Formula VIII-a. This amide can react with Lawesson's reagent in a solvent such as tetrahydrofuran to form the desired product of Structural Formula VIII-b.

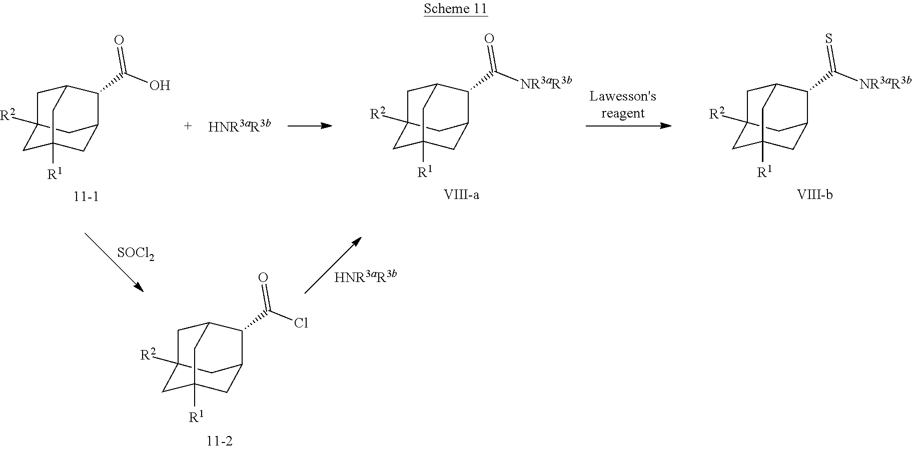

Scheme 12 depicts synthesis of adamantane carboxylic acids useful in preparation of compounds of the invention as described in Schemes 1, 5, and 6. Bridgehead hydroxylation of adamantane acid 12 using an oxidizing agent such as potassium permanganate in the presence of a base such as potassium hydroxide in a solvent such as water followed by esterification and subsequent reaction of corresponding hydroxy adamantane with benzene in the presence of triflic acid can afford compound 13. Reaction of compound 13 with fluorinating reagent such as diethylaminosulfur trifluoride (DAST) in a solvent such as dichloromethane followed by ester hydrolysis using a base such as LiOH in a solvent such as aqueous methanol or THE can provide adamantane carboxylic acid 14. Reduction of ketone in compound 13 using a reducing agent such as NaBH₄ and subsequent treatment of corresponding alcohol with Tf₂O in the presence of a base such as N,N-diisopropylethylamine in a solvent such as dichloromethane can provide triflate 15. The reaction of triflate 15 with R″SNa or R″ONa (R″=alkyl, cycloalkyl, or aryl) formed from thiols R″SH or alcohols R″OH in the presence of a base such as NaH in a solvent such as DMF or THE followed by ester hydrolysis can afford carboxylic acids 16 and 17, respectively.

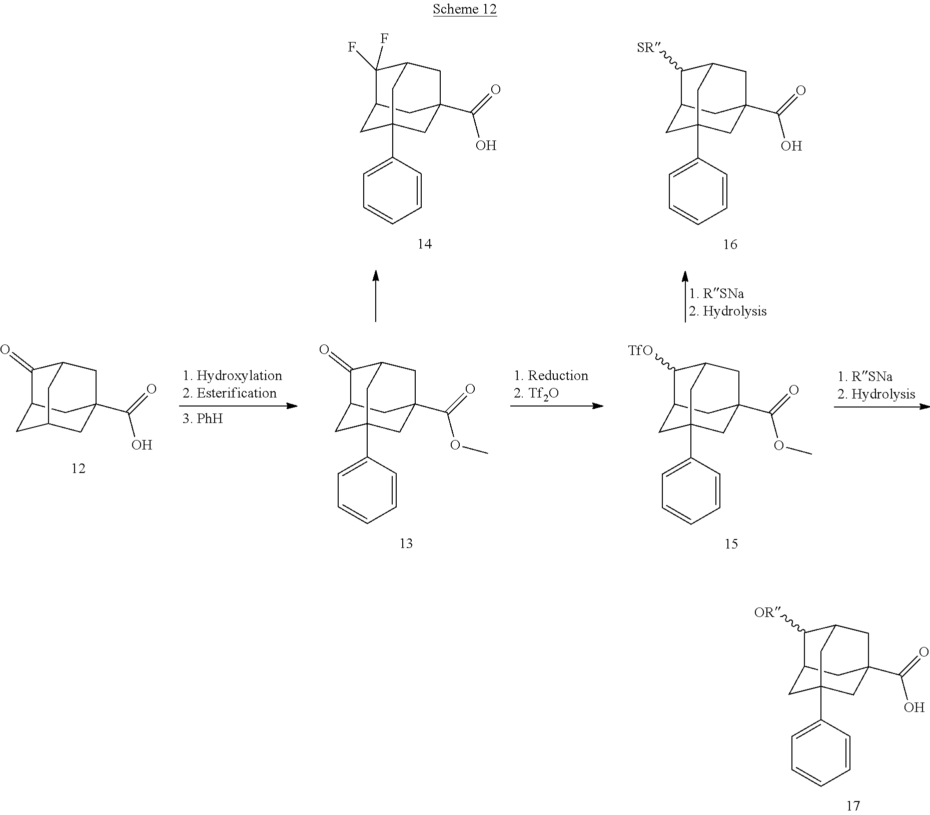

Scheme 13 depicts synthesis of adamantane carboxylic acids useful in preparation of compounds of the invention as described in Schemes 1, 5, and 6. Wittig reaction of ketone 13 with the ylide generated from a phosphonium salt 18 (X=Cl, Br, or I, and n=0 or 1) in the presence of a base such as lithium bis(trimethylsilyl)amide in a solvent such as diethyl ether or THE followed by deprotection of 1,3-dioxolane to aldehyde using catalytic amount of acid such as p-toluenesullfonic acid in a solvent such as acetone can afford compound 19. Reduction of the aldehyde and alkene using a reducing agent such as hydrogen gas in the presence of a catalyst such as palladium on carbon in a solvent such as methanol or ethanol followed by deoxy-chlorination of alcohol using trivalent phosphorous compound such as Ph₃P and an electrophilic halogen-containing agent such as n-Bu₄NI in a solvent such as dichloroethane and subsequent ester hydrolysis can provide carboxylic acid 21. Reaction of compound 19 with fluorinating reagent such as diethylaminosulfur trifluoride (DAST) in a solvent such as dichloromethane followed by reduction of the alkene using a reducing agent such as hydrogen gas in the presence of a catalyst such as palladium on carbon in a solvent such as methanol or ethanol, and subsequent ester hydrolysis using a base such as LiOH in a solvent such as aqueous methanol or THE can provide adamantane carboxylic acid 20. Reaction of compound 19 (n=0) with lithium dialkylcuprate R″₂CuLi (R′″=alkyl) in the presence of Me₃SiCl in a solvent such as THE followed by hydrolysis of the resultant silyl enol ether under acidic conditions can provide compound 23. Reaction of compound 23 with fluorinating reagent such as diethylaminosulfur trifluoride (DAST) in a solvent such as dichloromethane followed by ester hydrolysis using a base such as LiOH in a solvent such as aqueous methanol or THE can provide adamantane carboxylic acid 22. Reduction of the aldehyde in compound 23 using a reducing agent such as hydrogen gas in the presence of a catalyst such as palladium on carbon in a solvent such as methanol or ethanol followed by deoxy-chlorination of alcohol using trivalent phosphorous compound such as Ph₃P and an electrophilic halogen-containing agent such as n-Bu₄NI in a solvent such as dichloroethane can provide ester 24. Hydrolysis of ester 24 using a base such as LiOH in a solvent such as aqueous methanol or THE can provide adamantane carboxylic acid 25. Reduction of chloroethyl group in 24 to ethyl using a reducing agent such as Zn dust in a solvent such as acetic acid or hydrogen gas in the presence of a catalyst such as palladium on carbon in a solvent such as methanol or ethanol followed by ester hydrolysis can afford adamantane carboxylic acid 26.

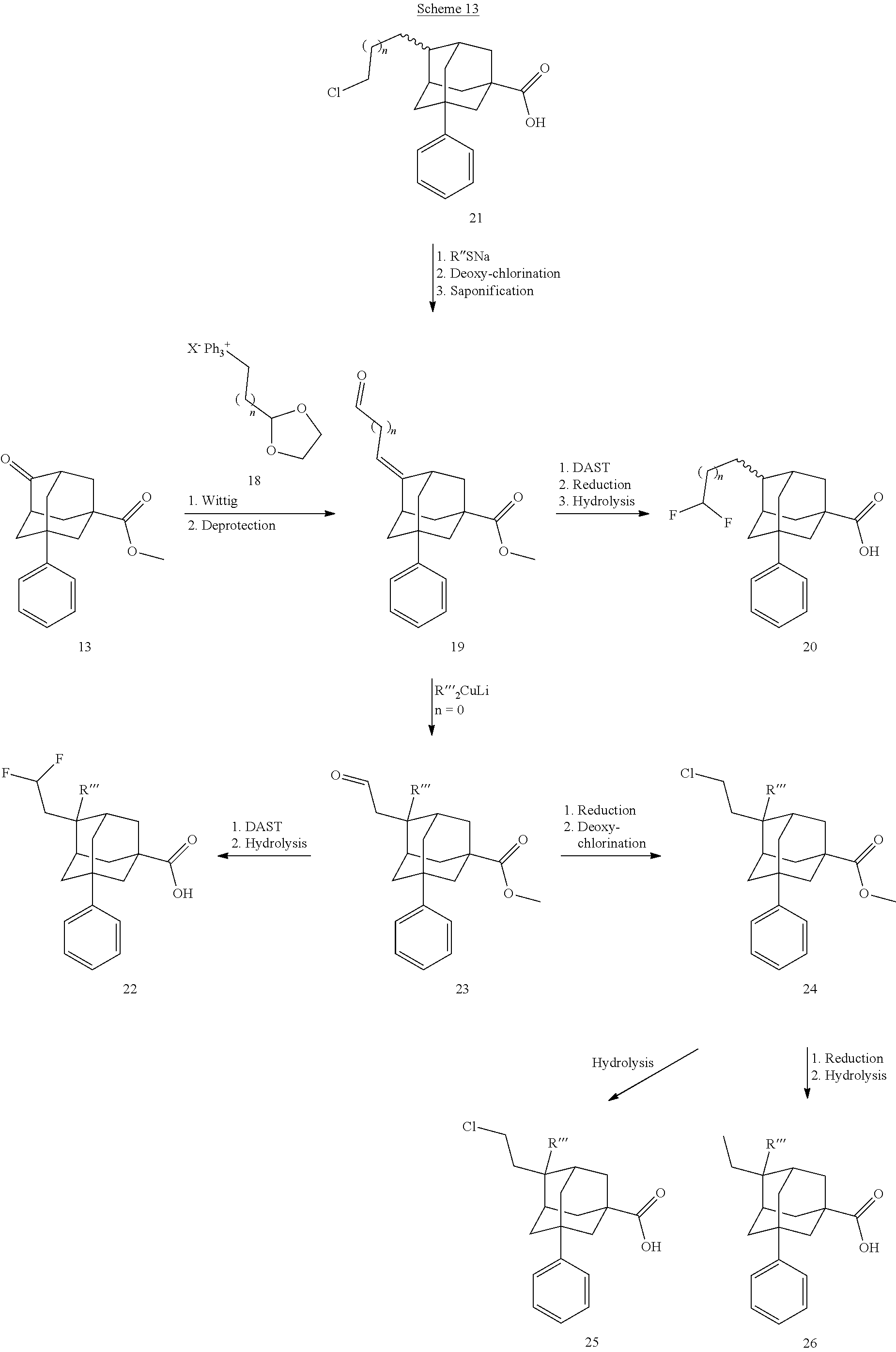

Scheme 14 depicts synthesis of adamantane carboxylic acids useful in preparation of compounds of the invention as described in Schemes 1, 5, and 6. Wittig reaction of ketone 13 with the ylide generated from a phosphonium salt 27 (X=Cl, Br, or I) in the presence of a base such as lithium bis(trimethylsilyl)amide in a solvent such as diethyl ether or THE followed by hydrolysis of the resultant methyl enol ether under acidic conditions, for example, using trifluoroacetic acid in a solvent such as dichloromethane can afford compound 28. Reduction of aldehyde in compound 28 using a reducing agent such as sodium borohydride in a solvent such as methanol or ethanol followed by deoxy-chlorination of alcohol using trivalent phosphorous compound such as Ph₃P and an electrophilic halogen-containing agent such as n-Bu₄NI in a solvent such as dichloroethane, and subsequent ester hydrolysis can provide adamantane carboxylic acid 29. Reaction of compound 28 with fluorinating reagent such as diethylaminosulfur trifluoride (DAST) in a solvent such as dichloromethane followed by ester hydrolysis using a base such as LiOH in a solvent such as aqueous methanol or THE can provide adamantane carboxylic acid 34. Wittig reaction of ketone 13 with the ylide generated from a phosphonium salt 30 (X=Cl, Br, or I) in the presence of a base such as lithium bis(trimethylsilyl)amide in a solvent such as diethyl ether or THE followed by Simmons-Smith reaction of formed alkene with diiodomethane in the presence of metallic zinc and copper (Zn/Cu) in a solvent such as dichloromethane can afford ester 32. Hydrolysis of ester 32 using a base such as LiOH in a solvent such as aqueous methanol or THE can provide adamantane carboxylic acid 33. Hydrogenation of cyclopropane in compound 32 using hydrogen gas in the presence of a catalyst such as palladium on carbon in a solvent such as methanol or ethanol followed by ester hydrolysis can provide adamantane carboxylic acid 31.

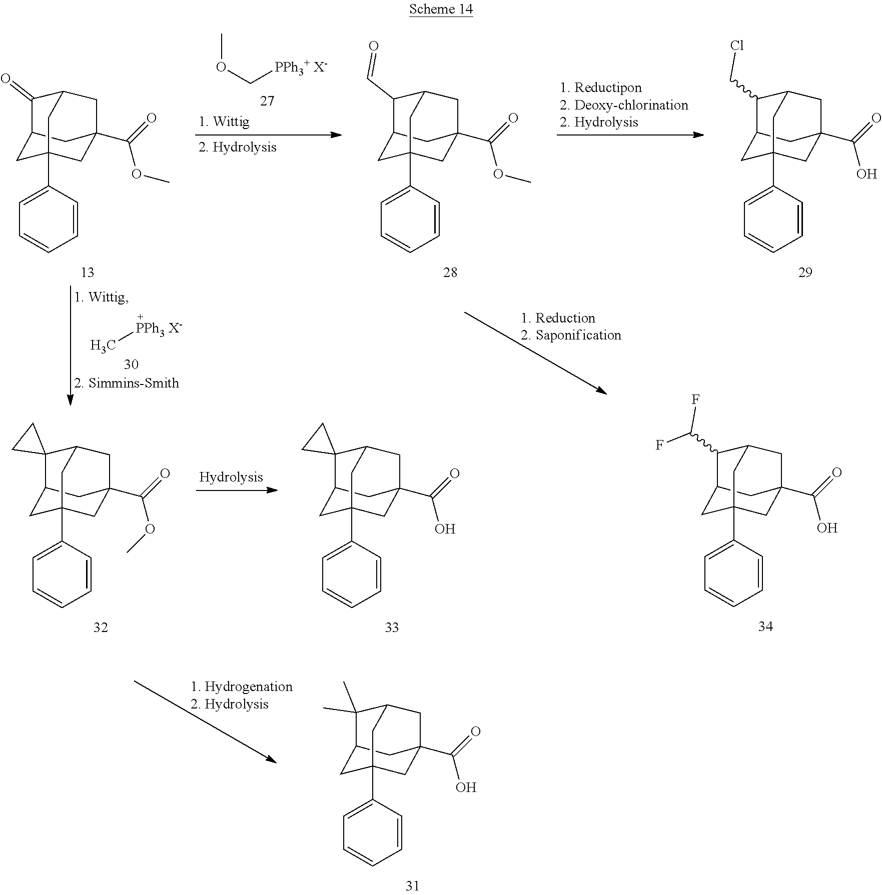

Scheme 15 depicts synthesis of adamantane carboxylic acid 35 useful in preparation of compounds of the invention as described in Schemes 2, 10, and 11. Van Leusen reaction of ketone 13 with tosylmethyl isocyabide (TosMIC) in the presence of a base such as potassium tert-butoxide in a mixture of solvents such as dimethoxyethane and ethanol followed by nitrile hydrolysis to amide, for example using hydrobromic acid solution in glacial acetic acid, and further hydrolysis of amide under acidic conditions can provide adamantane carboxylic acid 35.

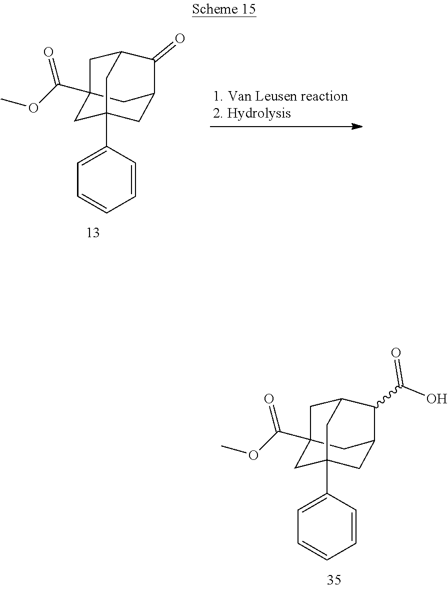 [SMILES: COC(=O)C12CC3CC(c4ccccc4)(CC(C1)C3=O)C2 ; COC(=O)C12CC3CC(c4ccccc4)(CC(C1)C3C(=O)O)C2]

Mixtures of isomers in Schemes 1, 2, 7, and 12-15 can be separated using well-known HPLC or crystallization techniques.

### Preparation of Intermediates

### (1 S,3R,5R,7S)-3-methyl-5-phenyladamantane-1-carboxylic acid and (1R,3S,5S,7R)-3-methyl-5-phenyladamantane-1-carboxylic acid

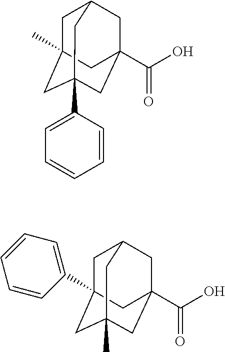 [SMILES: C[C@]12CC3CC(C(=O)O)(C1)C[C@@](c1ccccc1)(C3)C2 ; C[C@@]12CC3CC(C(=O)O)(C1)C[C@](c1ccccc1)(C3)C2]

The title compounds were obtained by chiral separation of racemic 3-methyl-5-phenyladamantane-1-carboxylic acid (commercially available from Enamine, product number EN300-54568) on a prep. Agilent 1200 (Chiralpak AS 20×250 mm, 10 um; mobile phase: n-hexane-2-propanol-TFA, 97-3-0; flow rate: 13 mL/min, injection: 40 mg). Each enantiomer was separately converted to the corresponding methyl ester whose optical rotation was compared with published data [Aoyama, M; Hara, S. Synthesis of optically active fluoroadamantanederivatives having different substituents on the tert-carbons and its use as non-racemizable source for new optically active adamantane derivatives. Tetrahedron 2013, 69, 10357-10360; Plewe, M. et al. PCT patent application, publication number PCT/US2018/041715, 11 Jul. 2018; WO 2019/018185, 24 Jan. 2019]. The above paper and patent application are herein incorporated by reference in their entirety for all purposes.

### rac-3-(methoxycarbonyl)-5-phenyladamantane-1-carboxylic acid

### Step 1: 1,3-dimethyl 5-phenyladamantane-1,3-dicarboxylate

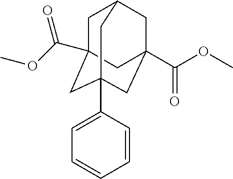

The title compound was prepared from 1,3-dimethyl 5-hydroxyadamantane-1,3-dicarboxylate and benzene following the procedure described in PCT patent application, publication number PCT/US2018/041715, 11 Jul. 2018; WO 2019/018185, 24 Jan. 2019 herein incorporated by reference in its entirety for all purposes. ¹H NMR (500 MHz, CDCl₃) δ 7.37-7.30 (m, 4H), 7.20 (t, 1H), 3.67 (s, 6H), 2.38-2.35 (m, 1H), 2.07 (br. s, 2H), 2.05-1.99 (m, 4H), 1.92-1.85 (m, 6H).

### Step 2: rac-3-(methoxycarbonyl)-5-phenyladamantane-1-carboxylic acid

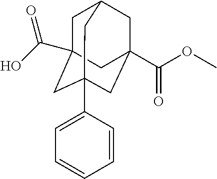 [SMILES: COC(=O)C12CC3CC(C(=O)O)(C1)CC(c1ccccc1)(C3)C2]

The title compound was prepared by reacting 1,3-dimethyl 5-phenyladamantane-1,3-dicarboxylate with 1 eq. NaOH following the procedure described in PCT patent application, publication number PCT/US2018/041715, 11 Jul. 2018; WO 2019/018185, 24 Jan. 2019 herein incorporated by reference in its entirety for all purposes.

### rac-3-bromo-5-phenyladamantane-1-carboxylic acid

### Step 1: methyl rac-3-bromo-5-phenyladamantane-1-carboxylate

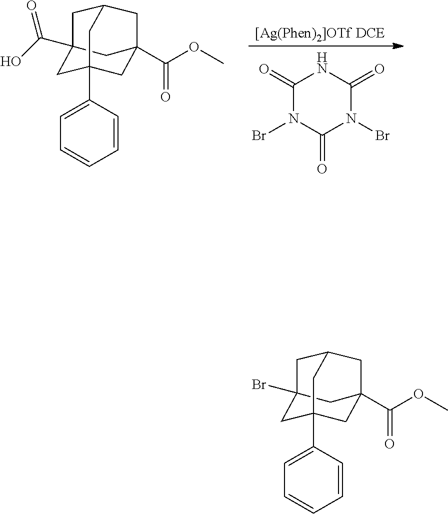

To a solution of rac-3-(methoxycarbonyl)-5-phenyladamantane-1-carboxylic acid (0.5 g, 1.6 mmol) in 1,2-dichloroethane (30 mL) is added dibromoisocyanuric acid (0.454 g, 1.6 mmol) and the complex [Ag(Phen)₂]OTf (0.098 g, 0.16 mmol). The mixture is degassed by bubbling nitrogen for 3 minutes, and then stoppered under a positive pressure of nitrogen. The reaction is stirred overnight at 60° C., at which time the solution is filtered through celite and the filtrate added directly to a SiO₂ column and eluted (hexanes:ethyl acetate 8:2) to give 110 mg of the title compound as clear oil, which was used in the next step.

### Step 2: rac-3-bromo-5-phenyladamantane-1-carboxylic acid

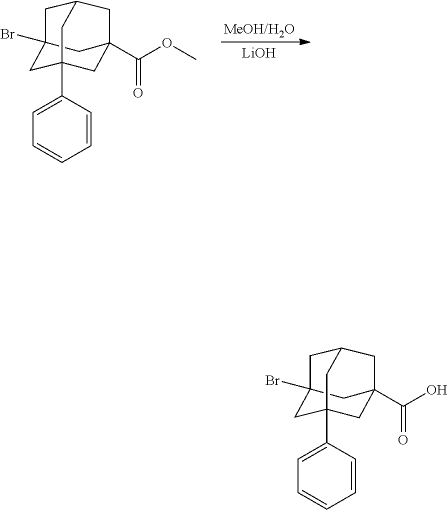

To a solution of methyl rac-3-bromo-5-phenyladamantane-1-carboxylate (110 mg) in 1 mL of methanol is added 0.5 mL of water, and 30 mg of LiOH. The mixture is stirred overnight, at which time it is diluted with 1 M aq. HCl, and extracted 3× with ethyl acetate. The organic layer is dried over Na₂SO₄ and evaporated to give the title compound (97 mg) as a white solid, which is used without further purification. LC/MS m/z: 333.30 (M−H, ⁷⁹Br)⁻, 335.32 (M−H, ⁸¹Br)⁻

### 5-Phenyladamantane-2-carboxylic acid

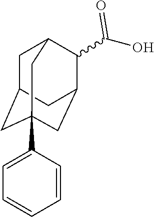

The title compound was prepared following the procedure described in PCT patent application, publication number PCT/US2017/013560, 13 Jan. 2017; WO 2017/127306, 27 Jul. 2017 herein incorporated by reference in its entirety for all purposes. The absolute cis/trans configuration of the single carboxylic acid product is unknown. ¹³C NMR (125 MHz, CDCl₃) δ: 180.35, 150.64, 128.45, 125.96, 125.01, 49.04, 48.76, 43.76, 43.33, 39.16, 37.43, 35.83, 32.96, 30.40, 28.41.

### methyl 6-oxo-3-phenyladamantane-1-carboxylate

### Step 1: 3-hydroxy-6-oxoadamantane-1-carboxylic acid

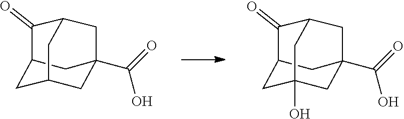 [SMILES: O=C1C2CC3CC1CC(C(=O)O)(C3)C2 ; O=C1C2CC3(O)CC1CC(C(=O)O)(C2)C3]

To a solution of 200 mL water and 7.22 g (129 mmol, 1 Eq) of KOH is added 25 g (129 mmol, 1 Eq) of 6-oxoadamantane-1-carboxylic acid. KMnO₄ (101.8 g, 5 Eq) is then added portionwise with strong stirring. After addition, the mixture is stirred vigorously at 60° C. for 36 hours, at which time the solution has turned from bright purple to black. The mixture is cooled to room temperature, filtered through celite, and the filter cake washed with water. The murky filtrate was clarified by the addition of a small amount of sodium bisulfite, resulting in a clear yellow solution. Ice is added to the filtrate and concentrated HCl is added slowly via addition funnel until the pH=3 (32 mL HCl). The resulting solution is stirred for 10 minutes, at which time the white precipitate, consisting of pure starting material, is filtered. The filtrate is then saturated with solid sodium chloride and extracted 3 times with 10% methanol in ethyl acetate. The organic extracts are dried over sodium sulfate, and evaporated to give 10.58 g of white solid, which consists of a mixture of unreacted starting material and the title compound. LC/MS m/z: 209.3 [M−H]⁻, 419.2 [2M−H]⁻

### Step 2: methyl 3-hydroxy-6-oxoadamantane-1-carboxylate

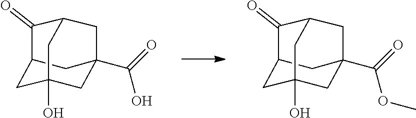 [SMILES: O=C1C2CC3(O)CC1CC(C(=O)O)(C2)C3 ; COC(=O)C12CC3CC(O)(CC(C1)C3=O)C2]

Crude product from the previous step (13.38 g, 59.7 mmol) is dissolved in DMF (20 mL), and potassium carbonate (16.49 g, 119.5 mmol) is added in one portion. The mixture is stirred at 40° C. for 30 minutes, and methyl iodide (5.61 mL, 90 mmol) is added slowly. The mixture is stirred overnight at 40° C., then filtered through celite and the filter cake washed with ethyl acetate. The filtrate is evaporated to remove ethyl acetate, and the resulting DMF solution is added directly to a flash chromatography column equilibrated with 6/4 hexanes/ethyl acetate. The compound is eluted with the same 6/4 mixture. The compound co-elutes with a small amount of DMF, which is removed by repeated azeotropic distillation with toluene, giving 3.82 g of the title compound as a white solid. 1H NMR (500 MHz, CDCl3) δ 3.70 (s, 3H), 2.64 (br: s, 2H), 2.17 (br:s. 1H), 2.15 (s, 3H), 2.08 (t, 4H), 1.91 (dd, 2H), 1.78 (br:s, 1H)

### Step 3: methyl 6-oxo-3-phenyladamantane-1-carboxylate

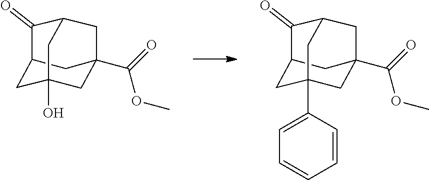 [SMILES: COC(=O)C12CC3CC(O)(CC(C1)C3=O)C2 ; COC(=O)C12CC3CC(c4ccccc4)(CC(C1)C3=O)C2]

To a solution of methyl 3-hydroxy-6-oxoadamantane-1-carboxylate (2.75 g, 12.28 mmol) in benzene (20 mL) is added triflic acid (1.08 mL, 12.28 mmol) dropwise. The resulting solution is stirred under nitrogen at 85° C. for 12 hours, then cooled to room temperature. The benzene solution is added directly to a flash chromatography column equilibrated with 8/2 hexanes/ethyl acetate and eluted with the same mixture to give 560 mg of the title compound as a yellow oil that slowly solidifies. 1H NMR (500 MHz, CDCl3) 7.38-7.33 (m, 4H), 7.26-7.23 (m, 1H), 3.71 (s, 3H), 2.72 (br:s, 2H), 2.34 (s, 2H), 2.28-2.24 (m, 6H), 2.22-2.18 (m, 3H).

### 6-methylene-3-phenyladamantane-1-carboxylic acid

### Step 1: methyl 6-methylene-3-phenyladamantane-1-carboxylate

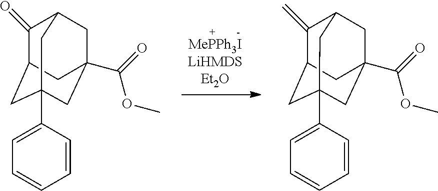

To a solution of methyltriphenylphosphonium iodide (125 mg, 0.31 mmol) in diethyl ether (3 mL) at 0° C. under nitrogen is added 1 M LiHMDS solution (0.31 mmol, 0.31 mL) dropwise. The orange mixture is stirred for 1 hour warming to room temperature, at which point a solution of methyl 6-oxo-3-phenyladamantane-1-carboxylate (80 mg, 0.28 mmol) in diethyl ether (1 mL) is added dropwise. The resulting solution is stirred at 50° C. overnight, then filtered through celite and the filter cake washed with ethyl acetate. The filtrate is washed once with water and brine, dried over sodium sulfate, and evaporated. The crude product is purified via flash chromatography with 9/1 hexane/ethyl acetate eluent, to give 70 mg of the title compound as a clear oil. 1H NMR (500 MHz, CDCl3) 7.37-7.31 (m, 4H), 7.20 (t, 1H), 4.65 (s, 2H), 3.68 (s, 3H), 2.75 (s, 2H), 2.16 (s, 2H), 2.01 (br:s. 4H), 1.99 (d, 4H),

### Step 2: 6-methylene-3-phenyladamantane-1-carboxylic acid

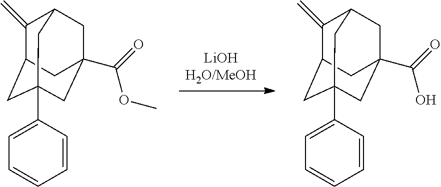

70 mg of the starting material is dissolved in methanol (2 mL), and 0.5 mL water is added. 50 mg of LiOH is then added in one portion, and the mixture stirred overnight. The solution is then evaporated and the residue dissolved in 1M HCl and extracted 3 times with ethyl acetate. The organic extracts are evaporated to give 58 mg of the title compound as a white solid, which is not purified or analyzed further.

It is interesting to note that compounds of Structural Formula IX (wherein R⁸ is not hydrogen) are racemic mixtures, which may be separated by chiral HPLC or other conventional methods of separating enantiomers [Walborsky, H. M.; Gawronska, K., and Gawronski, J. K. Synthesis and Chiroptical Properties of [symbol] -Substituted Rigid and Conformationally Flexible Systems Having 1,3-Diene and α,β-Unsaturated Carbonyl Chromophores. The Planar Diene Rule. J. Am. Chem. Soc. (1987) 109:6719-6726]. The above paper is herein incorporated by reference in its entirety for all purposes.

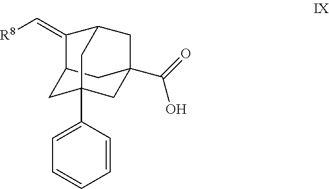

### 6-ethylidene-3-phenyladamantane-1-carboxylic acid

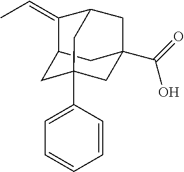 [SMILES: CC=C1C2CC3(C(=O)O)CC1CC(c1ccccc1)(C2)C3]

The title compound was prepared from methyl 6-oxo-3-phenyladamantane-1-carboxylate and ethyltriphenylphosphonium iodide in the same manner as described above for 6-methylene-3-phenyladamantane-1-carboxylic acid.

### 3-phenyl-6-propylideneadamantane-1-carboxylic acid

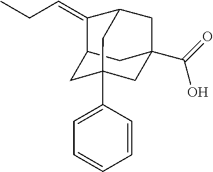 [SMILES: CCC=C1C2CC3(C(=O)O)CC1CC(c1ccccc1)(C2)C3]

The title compound was prepared from methyl 6-oxo-3-phenyladamantane-1-carboxylate and propyltriphenylphosphonium iodide in the same manner as described above for 6-methylene-3-phenyladamantane-1-carboxylic acid.

### 6-(cyclopropylmethylene)-3-phenyladamantane-1-carboxylic acid

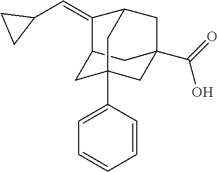 [SMILES: O=C(O)C12CC3CC(c4ccccc4)(CC(C1)C3=CC1CC1)C2]

The title compound was prepared from methyl 6-oxo-3-phenyladamantane-1-carboxylate and (cyclopropylmethyl)triphenylphosphonium bromide in the same manner as described above for 6-methylene-3-phenyladamantane-1-carboxylic acid.

### 6-(benzylidene)-3-phenyladamantane-1-carboxylic acid

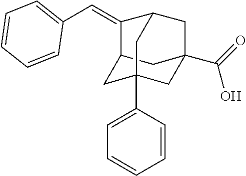 [SMILES: O=C(O)C12CC3CC(c4ccccc4)(CC(C1)C3=Cc1ccccc1)C2]

The title compound was prepared from methyl 6-oxo-3-phenyladamantane-1-carboxylate and benzyltriphenylphosphonium bromide in the same manner as described above for 6-methylene-3-phenyladamantane-1-carboxylic acid.

### 6-(3-methylbutylidene)-3-phenyladamantane-1-carboxylic acid

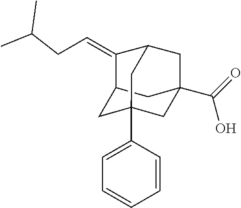 [SMILES: CC(C)CC=C1C2CC3(C(=O)O)CC1CC(c1ccccc1)(C2)C3]

The title compound was prepared from methyl 6-oxo-3-phenyladamantane-1-carboxylate and (3-methylbutyl)triphenylphosphonium bromide in the same manner as described above for 6-methylene-3-phenyladamantane-1-carboxylic acid.

### 6-(3-methylbut-2-en-1-ylidene)-3-phenyladamantane-1-carboxylic acid

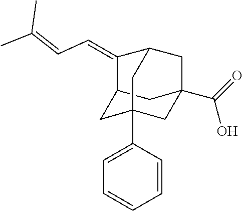 [SMILES: CC(C)=CC=C1C2CC3(C(=O)O)CC1CC(c1ccccc1)(C2)C3]

The title compound was prepared from methyl 6-oxo-3-phenyladamantane-1-carboxylate and (3,3-dimethylallyl)triphenylphosphonium bromide in the same manner as described above for 6-methylene-3-phenyladamantane-1-carboxylic acid.

### 6-butylidene-3-phenyladamantane-1-carboxylic acid

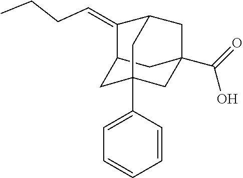 [SMILES: CCCC=C1C2CC3(C(=O)O)CC1CC(c1ccccc1)(C2)C3]

The title compound was prepared from methyl 6-oxo-3-phenyladamantane-1-carboxylate and butyltriphenylphosphonium bromide in the same manner as described above for 6-methylene-3-phenyladamantane-1-carboxylic acid.

### 6-allylidene-3-phenyladamantane-1-carboxylic acid

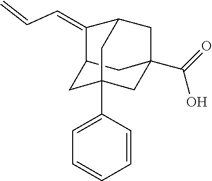 [SMILES: C=CC=C1C2CC3(C(=O)O)CC1CC(c1ccccc1)(C2)C3]

The title compound was prepared from methyl 6-oxo-3-phenyladamantane-1-carboxylate and allyltriphenylphosphonium bromide in the same manner as described above for 6-methylene-3-phenyladamantane-1-carboxylic acid.

### 3-phenyl-6-(2-phenylethylidene)adamantane-1-carboxylic acid

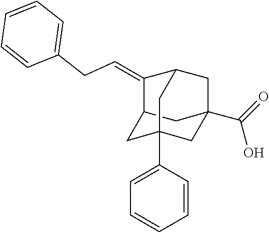 [SMILES: O=C(O)C12CC3CC(c4ccccc4)(CC(C1)C3=CCc1ccccc1)C2]

The title compound was prepared from methyl 6-oxo-3-phenyladamantane-1-carboxylate and phenethyltriphenylphosphonium bromide in the same manner as described above for 6-methylene-3-phenyladamantane-1-carboxylic acid.

### methyl 6-(2-fluoroethyl)-3-phenyladamantane-1-carboxylate

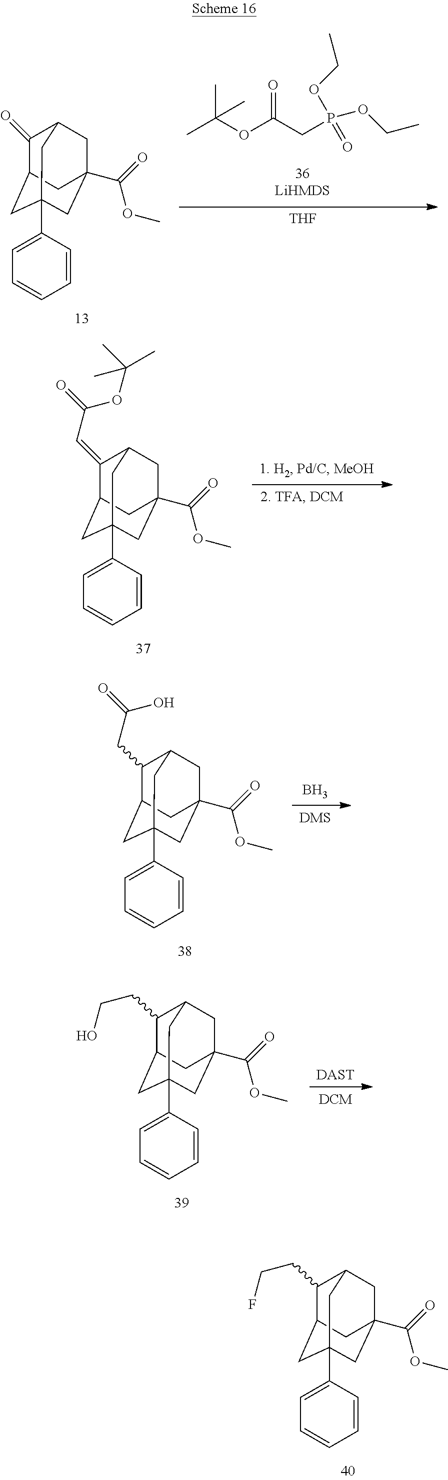

The title compound was custom synthesized for us from methyl 6-oxo-3-phenyladamantane-1-carboxylate using the synthetic route outlined in Scheme 16. The reaction of methyl 6-oxo-3-phenyladamantane-1-carboxylate 13 with tert-butyl 2-(diethoxyphosphoryl)acetate 36 in the presence of LiHMDS followed by alkene hydrogenation and tert-butyl group cleavage under acidic conditions afforded acid 38. The reduction of acid 38 with BH₃ provided alcohol 39. Treatment of alcohol 39 with DAST afforded compound methyl 6-(2-fluoroethyl)-3-phenyladamantane-1-carboxylate 40. LC/MS m/z: 317.2 (M+H)⁺

### 6-(2-fluoroethyl)-3-phenyladamantane-1-carboxylic acid

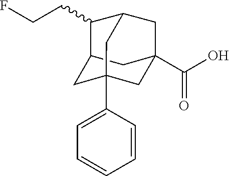 [SMILES: O=C(O)C12CC3CC(c4ccccc4)(CC(C1)C3CCF)C2]

The title compound was synthesized from methyl 6-(2-fluoroethyl)-3-phenyladamantane-1-carboxylate in the same manner as described above for 6-methylene-3-phenyladamantane-1-carboxylic acid.

### methyl 6-(2-chloroethyl)-3-phenyladamantane-1-carboxylate

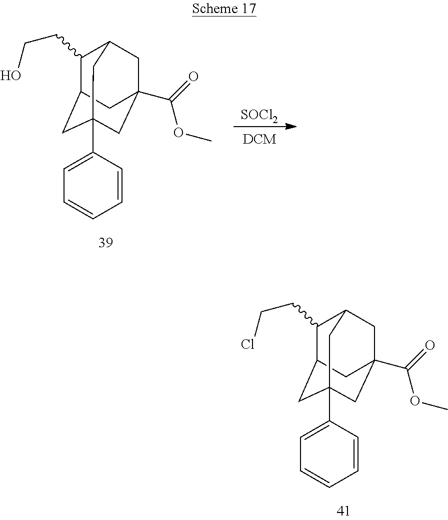

The title compound was custom synthesized for us by treating methyl 6-(2-hydroxyethyl)-3-phenyladamantane-1-carboxylate with SOCl₂ in DCM as outlined in Scheme 17. LC/MS m/z: 333.2 (M+H)⁺

### 6-(2-chloroethyl)-3-phenyladamantane-1-carboxylic acid

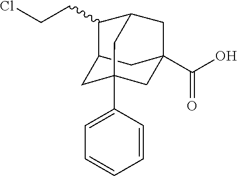 [SMILES: O=C(O)C12CC3CC(c4ccccc4)(CC(C1)C3CCCl)C2]

The title compound was synthesized from methyl 6-(2-chloroethyl)-3-phenyladamantane-1-carboxylate in the same manner as described above for 6-methylene-3-phenyladamantane-1-carboxylic acid.

### methyl 6-(2,2-difluoroethyl)-3-phenyladamantane-1-carboxylate

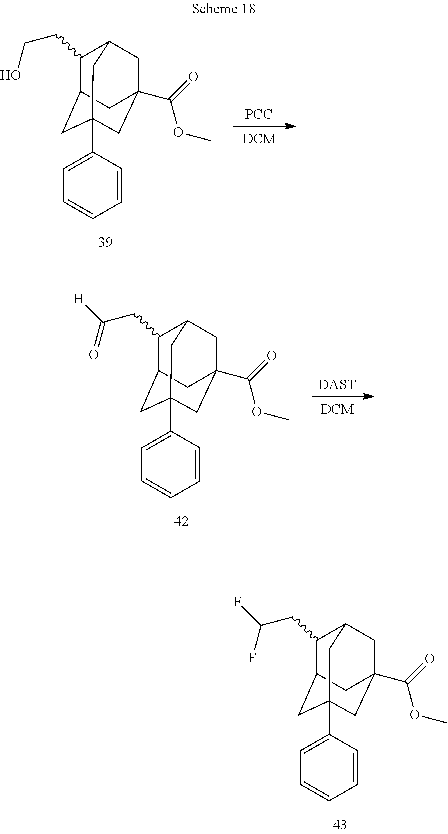

The title compound was custom synthesized for us from methyl 6-(2-hydroxyethyl)-3-phenyladamantane-1-carboxylate using the synthetic route outlined in Scheme 18. Oxidation of methyl 6-(2-hydroxyethyl)-3-phenyladamantane-1-carboxylate 39 to aldehyde 42 using pyridinium chlorochromate (PCC) in DCM followed by the reaction of aldehyde 42 with diethylaminosulfur trifluoride (DAST) afforded methyl 6-(2,2-difluoroethyl)-3-phenyladamantane-1-carboxylate 43. LC/MS m/z: 335.4 (M+H)⁺

### 6-(2,2-difluoroethyl)-3-phenyladamantane-1-carboxylic acid

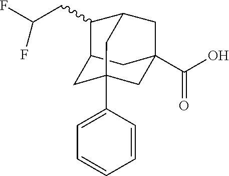 [SMILES: O=C(O)C12CC3CC(c4ccccc4)(CC(C1)C3CC(F)F)C2]

The title compound was synthesized from methyl 6-(2,2-difluoroethyl)-3-phenyladamantane-1-carboxylate in the same manner as described above for 6-methylene-3-phenyladamantane-1-carboxylic acid.

### EXAMPLES

### Example A1: ((S)-4-amino-3,3-dimethylpiperidin-1-yl)((1 S,3R,5R,7S)-3-methyl-5-phenyladamantan-1-yl)methanethione

### Step 1: tert-butyl ((S)-3,3-dimethyl-1-((1 S,3R,5R,7S)-3-methyl-5-phenyladamantane-1-carbonyl)piperidin-4-yl)carbamate

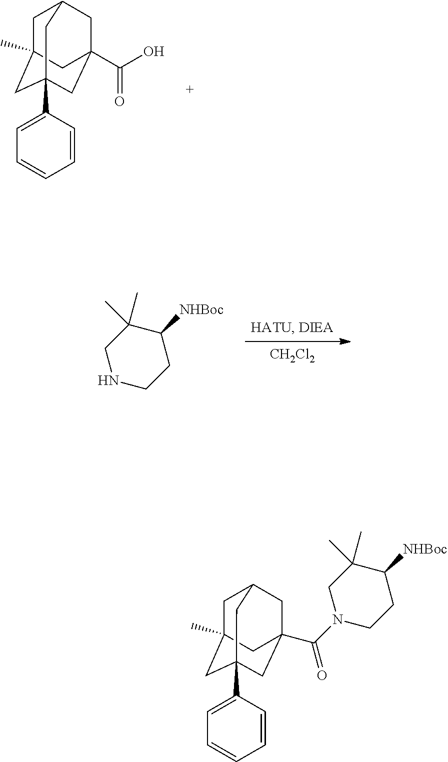 [SMILES: C[C@]12CC3CC(C(=O)O)(C1)C[C@@](c1ccccc1)(C3)C2 ; CC(C)(C)OC(=O)N[C@H]1CCNCC1(C)C ; "CH2Cl2" ; "HATU, DIEA" ; CC(C)(C)OC(=O)N[C@H]1CCN(C(=O)C23CC4C[C@@](C)(C2)C[C@](c2ccccc2)(C4)C3)CC1(C)C]

To a solution of (1 S,3R,5R,7S)-3-methyl-5-phenyladamantane-1-carboxylic acid (0.100 g, 0.37 mmol) in 2 mL of anhydrous CH₂Cl₂ was added HATU (0.182 g, 0.48 mmol) followed by N,N-diisopropylethylamine (0.16 mL, 0.92 mmol). The resulting reaction mixture was stirred at room temperature for 0.5 h, then tert-butyl (S)-(3,3-dimethylpiperidin-4-yl)carbamate (0.084 g, 0.37 mmol) was added. The reaction mixture was stirred at room temperature for 12 h to complete the reaction. Washed with water, dried (Na₂SO₄), filtered and evaporation of solvent under vacuum gave crude residue, which was purified by SiO₂ column chromatography to give the title compound. Yield: 0.144 g (82%). LC/MS m/z: 497.2 (M+H)⁺

### Step 2: ((S)-4-amino-3,3-dimethylpiperidin-1-yl)((1 S,3R,5R,7S)-3-methyl-5-phenyladamantan-1-yl)methanethione

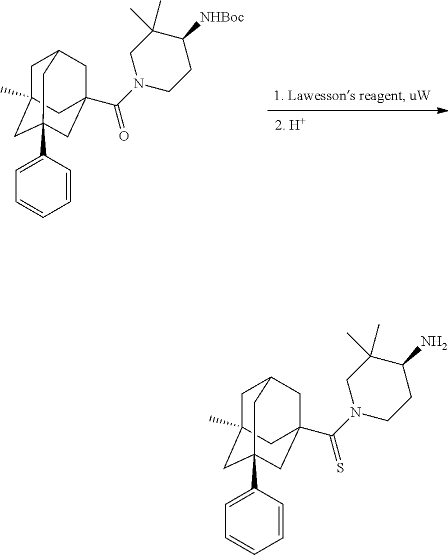

To a solution of tert-butyl ((S)-3,3-dimethyl-1-((1S,3R,5R,7S)-3-methyl-5-phenyladamantane-1-carbonyl)piperidin-4-yl)carbamate (0.012 g, 0.025 mmol) in 0.2 mL of THE was added Lawesson's reagent (0.010 g, 0.025 mmol) and the resulting reaction mixture was microwaved at 100° C. for 1 h. Solvent was removed under vacuum, then crude residue was dissolved in DCM (0.4 mL), and TFA (0.2 mL) was added. Stirred at room temperature for 2 h, solvent was evaporated and crude residue was purified using preparative HPLC to give the title compound. Yield: 0.007 g (70%). LC/MS m/z: 397.40 (M+H)⁺

Examples A2 and A4-A7 were prepared in the same manner as described above for ((S)-4-amino-3,3-dimethylpiperidin-1-yl)((1S,3R,5R,7S)-3-methyl-5-phenyladamantan-1-yl)methanethione (example A1) using (1 S,3R,5R,7S)-3-methyl-5-phenyladamantane-1-carboxylic acid and the appropriate amine as starting materials.

Examples B1 and B2 were prepared in the same manner as described above for ((S)-4-amino-3,3-dimethylpiperidin-1-yl)((1 S,3R,5R,7S)-3-methyl-5-phenyladamantan-1-yl)methanethione (example A1) using (1R,3S,5S,7R)-3-methyl-5-phenyladamantane-1-carboxylic acid and the appropriate amine as starting materials.

Examples C and C2 were prepared in the same manner as described above for ((S)-4-amino-3,3-dimethylpiperidin-1-yl)((l S,3R,5R,7S)-3-methyl-5-phenyladamantan-1-yl)methanethione (example A1) using racemic 3-methyl-5-phenyladamantane-1-carboxylic acid and the appropriate amine as starting materials.

Examples A3, B3 and C3 were prepared from the appropriate carboxylic acid and (3R)-1-azabicyclo [2.2.2]octan-3-amine in the same manner as described above for ((S)-4-amino-3,3-dimethylpiperidin-1-yl)((1S,3R,5R,7S)-3-methyl-5-phenyladamantan-1-yl)methanethione (example A1) omitting cleavage of the Boc-group under acidic conditions.

| Ex. | Starting materials | Product/Name | Analytical Data |
|---|---|---|---|
| B1 | tert-butyl (S)-(3,3- dimethylpiperidin- 4-yl)carbamate (1R,3S,5S,7R)-3- methyl-5- phenyladamantane- 1-carboxylic acid | 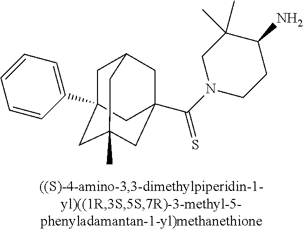 [SMILES: CC1(C)CN(C(=S)C23CC4C[C@](C)(C2)C[C@@](c2ccccc2)(C4)C3)CC[C@@H]1N] | LC/MS m/z: 397.40 (M + H)⁺ |
| C1 | tert-butyl (S)-(3,3- dimethylpiperidin- 4-yl)carbamate rac-3-methyl-5- phenyladamantane- 1-carboxylic acid | 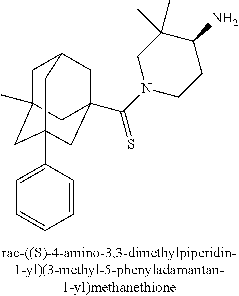 [SMILES: CC12CC3CC(C(=S)N4CC[C@H](N)C(C)(C)C4)(C1)CC(c1ccccc1)(C3)C2] | LC/MS m/z: 397.40 (M + H)⁺ |
| A2 | trans-tert-butyl N- (4- aminocyclohexyl) carbamate (1S,3R,5R,7S)-3- methyl-5- phenyladamantane- 1-carboxylic acid | 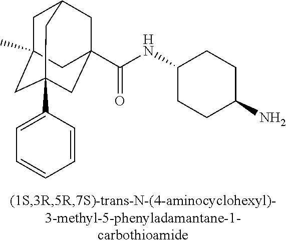 | LC/MS m/z: 383.37 (M + H)⁺ |
| B2 | trans-tert-butyl N- (4- aminocyclohexyl) carbamate (1R,3S,5S,7R)-3- methyl-5- phenyladamantane- 1-carboxylic acid | 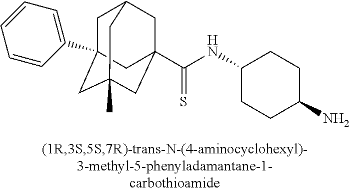 [SMILES: C[C@@]12CC3CC(C(=S)N[C@H]4CC[C@H](N)CC4)(C1)C[C@](c1ccccc1)(C3)C2] | LC/MS m/z: 383.34 (M + H)⁺ |
| C2 | trans-tert-butyl N- (4- aminocyclohexyl) carbamate rac-3-methyl-5- phenyladamantane- 1-carboxylic acid | 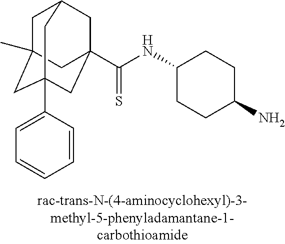 [SMILES: CC12CC3CC(C(=S)N[C@H]4CC[C@H](N)CC4)(C1)CC(c1ccccc1)(C3)C2] | LC/MS m/z: 383.34 (M + H)⁺ |
| A3 | (3R)-1-azabicyclo [2.2.2]octan-3- amine (1S,3R,5R,7S)-3- methyl-5- phenyladamantane- 1-carboxylic acid | 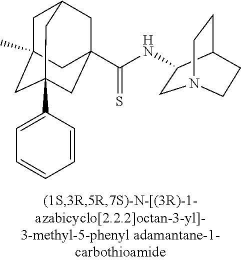 [SMILES: C[C@]12CC3CC(C(=S)N[C@H]4CN5CCC4CC5)(C1)C[C@@](c1ccccc1)(C3)C2] | LC/MS m/z: 395.38 (M + H)⁺ |
| B3 | (3R)-1-azabicyclo [2.2.2]octan-3- amine (1R,3S,5S,7R)-3- methyl-5- phenyladamantane- 1-carboxylic acid | 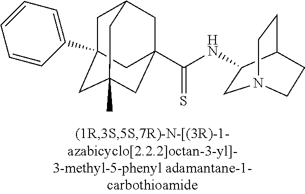 [SMILES: C[C@@]12CC3CC(C(=S)N[C@H]4CN5CCC4CC5)(C1)C[C@](c1ccccc1)(C3)C2] | LC/MS m/z: 395.39 (M + H)⁺ |
| C3 | (3R)-1-azabicyclo [2.2.2]octan-3- amine rac-3-methyl-5- phenyladamantane- 1-carboxylic acid | 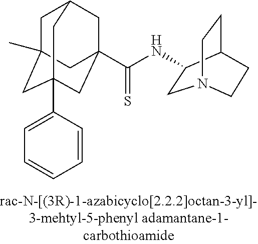 [SMILES: CC12CC3CC(C(=S)N[C@H]4CN5CCC4CC5)(C1)CC(c1ccccc1)(C3)C2] | LC/MS m/z: 395.39 (M + H)⁺ |
| A4 | tert-butyl piperidin- 4-ylcarbamate (1S,3R,5R,7S)-3- methyl-5- phenyladamantane- 1-carboxylic acid | 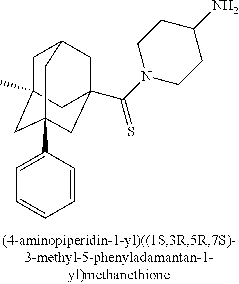 [SMILES: C[C@]12CC3CC(C(=S)N4CCC(N)CC4)(C1)C[C@@](c1ccccc1)(C3)C2] | LC/MS m/z: 369.32 (M + H)⁺ |
| A5 | cis-tert-butyl N-(4- aminocyclohexyl) carbamate (1S,3R,5R,7S)-3- methyl-5- phenyladamantane- 1-carboxylic acid | 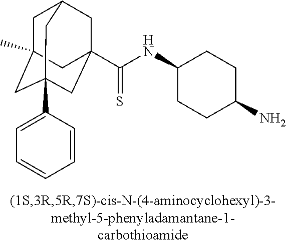 [SMILES: C[C@]12CC3CC(C(=S)N[C@H]4CC[C@@H](N)CC4)(C1)C[C@@](c1ccccc1)(C3)C2] | LC/MS m/z: 383.36 (M + H)⁺ |
| A6 | tert-butyl 4- aminopiperidine-1- carboxylate (1S,3R,5R,7S)-3- methyl-5- phenyladamantane- 1-carboxylic acid | 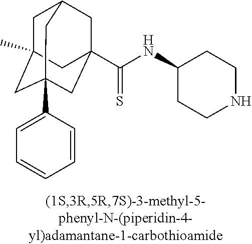 | LC/MS m/z: 369.29 (M + H)⁺ |
| A7 | trans-tert-butyl N- [(4- aminocyclohexyl) methyl]carbamate (1S,3R,5R,7S)-3- methyl-5- phenyladamantane- 1-carboxylic acid | 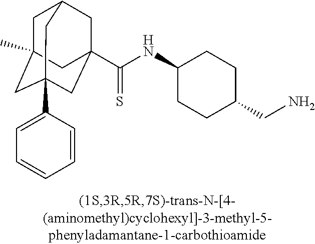 [SMILES: C[C@]12CC3CC(C(=S)N[C@H]4CC[C@H](CN)CC4)(C1)C[C@@](c1ccccc1)(C3)C2] | LC/MS m/z: 397.32 (M + H)⁺ |

### Example D1: trans-N-(4-aminocyclohexyl)-5-phenyladamantane-2-carbothioamide

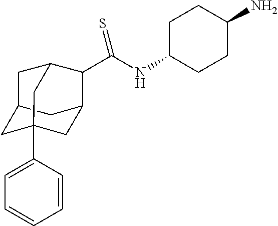

The title compound was prepared from 5-phenyladamantane-2-carboxylic acid and trans-tert-butyl N-(4-aminocyclohexyl)carbamate using the procedure described above for ((S)-4-amino-3,3-dimethylpiperidin-1-yl)((1 S,3R,5R,7S)-3-methyl-5-phenyladamantan-1-yl)methanethione (example A1). LC/MS m/z: 369.39 (M+H)⁺

### Example D2: trans-N-(4-aminocyclohexyl)-3-phenyl-6-propylideneadamantane-1-carbothioamide

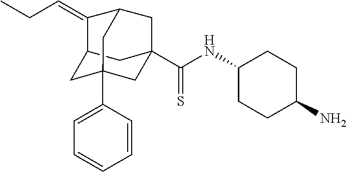

The title compound was prepared from 3-phenyl-6-propylideneadamantane-1-carboxylic acid and trans-tert-butyl N-(4-aminocyclohexyl)carbamate using the procedure described above for ((S)-4-amino-3,3-dimethylpiperidin-1-yl)((1 S,3R,5R,7S)-3-methyl-5-phenyladamantan-1-yl)methanethione (example A1). LC/MS m/z: 409.41 (M+H)⁺, 450.37 (M+H+CH₃CN)⁺

### Example E1: rac-trans-N-(4-aminocyclohexyl)-3-(chloromethyl)-5-phenyladamantane-1-carboxamide hydrochloride

### Step 1: rac-methyl-3-trans-4-((tert-butoxycarbonyl)amino)cyclohexyl)carbamoyl)-5-phenyladamantane-1-carboxylate

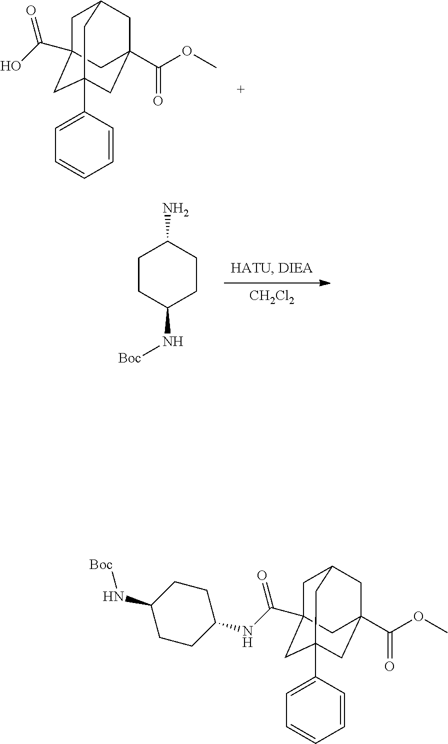 [SMILES: COC(=O)C12CC3CC(C(=O)O)(C1)CC(c1ccccc1)(C3)C2 ; CC(C)(C)OC(=O)N[C@H]1CC[C@H](N)CC1 ; "CH2Cl2" ; "HATU, DIEA" ; COC(=O)C12CC3CC(C(=O)N[C@H]4CC[C@H](NC(=O)OC(C)(C)C)CC4)(C1)CC(c1ccccc1)(C3)C2]

The title compound was prepared from rac-3-(methoxycarbonyl)-5-phenyladamantane-1-carboxylic acid and trans-tert-butyl N-(4-aminocyclohexyl)carbamate using the procedure described above for tert-butyl ((S)-3,3-dimethyl-1-((1S,3R,5R,7S)-3-methyl-5-phenyladamantane-1-carbonyl)piperidin-4-yl)carbamate. LC/MS m/z: 511.37 (M+H)⁺, 552.53 (M+H+CH₃CN)⁺

### Step 2: tert-butyl trans-4-rac-3-(hydroxymethyl)-5-phenyladamantane-1-carboxamido)cyclohexyl)carbamate

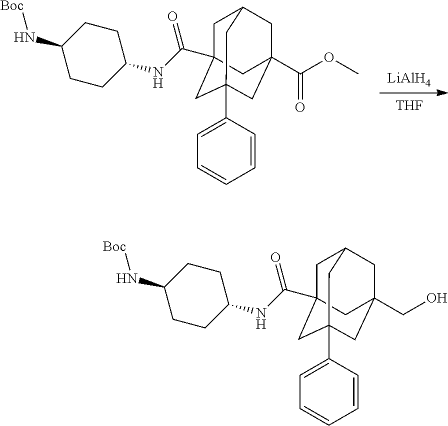 [SMILES: COC(=O)C12CC3CC(C(=O)N[C@H]4CC[C@H](NC(=O)OC(C)(C)C)CC4)(C1)CC(c1ccccc1)(C3)C2 ; "LiAlH4" ; CC(C)(C)OC(=O)N[C@H]1CC[C@H](NC(=O)C23CC4CC(CO)(C2)CC(c2ccccc2)(C4)C3)CC1 ; "THF"]

To a flame dried flask under N₂ is added lithium aluminum hydride (0.026 g, 0.69 mmol) and THE (5 mL). The mixture is cooled to 0° C., and a solution of rac-methyl-3-trans-4-((tert-butoxycarbonyl)amino)cyclohexyl)carbamoyl)-5-phenyladamantane-1-carboxylate (0.32 g, 0.63 mmol) in THE (1 mL) is added dropwise. After the addition, the mixture is stirred for one more hour at 0° C., at which time it is quenched with 2 mL of a saturated sodium sulfate solution. The mixture is stirred an additional half hour, at which time it is filtered through celite and the filtrate evaporated to give the title compound (0.310 g) as a white solid, which is used without further purification. LC/MS m/z: 483.43 (M+H)⁺, 524.48 (M+H+CH₃CN)⁺

### Step 3: rac-trans-N-(4-aminocyclohexyl)-3-(chloromethyl)-5-phenyladamantane-1-carboxamide hydrochloride

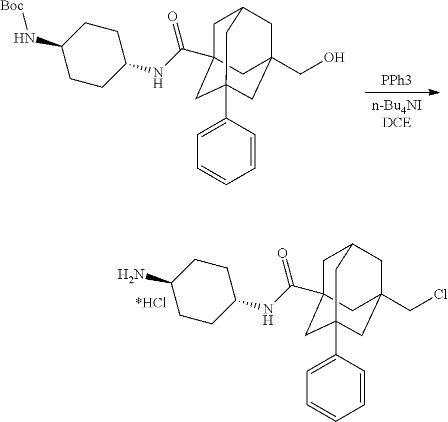 [SMILES: CC(C)(C)OC(=O)N[C@H]1CC[C@H](NC(=O)C23CC4CC(CO)(C2)CC(c2ccccc2)(C4)C3)CC1 ; "PPh3" ; "n-Bu4NIDCE" ; N[C@H]1CC[C@H](NC(=O)C23CC4CC(CCl)(C2)CC(c2ccccc2)(C4)C3)CC1]

To a microwave reactor vial is added tert-butyl trans-4-rac-3-(hydroxymethyl)-5-phenyladamantane-1-carboxamido)cyclohexyl)carbamate (0.03 g, 0.06 mmol), triphenylphosphine (0.019 g, 0.072 mmol), tetra n-butylammonium iodide (0.027 g, 0.072 mmol), and 1,2-dichloroethane (2 mL). The mixture is degassed by bubbling nitrogen for 3 minutes, and the vial is capped tightly and heated to 120° C. for 1 hour in a microwave reactor. The mixture is then evaporated, the residue dissolved in methanol, and purified via prep-HPLC. The purified product was then dissolved in 1 mL methanol containing 1.2 equivalents of HCl, and the solution evaporated to dryness in vacuo, to give the 8 mg of the title compound as a white hydrochloride salt. LC/MS m/z: 401.34 (M+H, ³⁵Cl)⁺, 403.42 (M+H, 37Cl)⁺

### Example E2: rac-((S)-4-amino-3,3-dimethylpiperidin-1-yl)(3-(chloromethyl)-5-phenyladamantan-1-yl)methanone

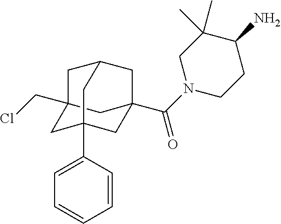 [SMILES: CC1(C)CN(C(=O)C23CC4CC(CCl)(C2)CC(c2ccccc2)(C4)C3)CC[C@@H]1N]

The title compound was prepared from rac-3-(methoxycarbonyl)-5-phenyladamantane-1-carboxylic acid and tert-butyl (S)-(3,3-dimethylpiperidin-4-yl)carbamate in the same manner as described above for rac-trans-N-(4-aminocyclohexyl)-3-(chloromethyl)-5-phenyladamantane-1-carboxamide hydrochloride (example E1, steps 1-3). LC/MS m/z: 415.33 (M+H, ³⁵Cl)⁺, 417.31 (M+H, ³⁷Cl)⁺

### Example E3: rac-trans-N-(4-aminocyclohexyl)-3-bromo-5-phenyladamantane-1-carboxamide

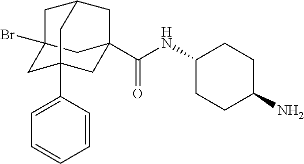 [SMILES: N[C@H]1CC[C@H](NC(=O)C23CC4CC(Br)(C2)CC(c2ccccc2)(C4)C3)CC1]

The title compound was prepared from rac-3-bromo-5-phenyladamantane-1-carboxylic acid and trans-tert-butyl N-(4-aminocyclohexyl)carbamate using the procedure described above for tert-butyl ((S)-3,3-dimethyl-1-((1 S,3R,5R,7S)-3-methyl-5-phenyladamantane-1-carbonyl)piperidin-4-yl)carbamate, followed by cleavage of the Boc-group under acidic conditions. LC/MS m/z: 431.25 (M+H, ⁷⁹Br)⁻, 433.27 (M+H, ⁸¹Br)⁻

### Example E4: (4-amino-3,3-difluoropiperidin-1-yl)((1 S,3R,5R,7S)-3-methyl-5-phenyladamantan-1-yl)methanone

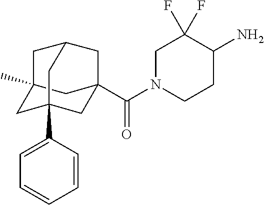 [SMILES: C[C@]12CC3CC(C(=O)N4CCC(N)C(F)(F)C4)(C1)C[C@@](c1ccccc1)(C3)C2]

The title compound was prepared from (1 S,3R,5R,7S)-3-methyl-5-phenyladamantane-1-carboxylic acid and tert-butyl (3,3-difluoropiperidin-4-yl)carbamate using the procedure described above for tert-butyl ((S)-3,3-dimethyl-1-((1S,3R,5R,7S)-3-methyl-5-phenyladamantane-1-carbonyl)piperidin-4-yl)carbamate, followed by cleavage of the Boc-group under acidic conditions. LC/MS m/z: 389.37 [M+H]⁺

### Example E5: (4-amino-3-(trifluoromethyl)piperidin-1-yl)((1S,3R,5R,7S)-3-methyl-5-phenyladamantan-1-yl)methanone

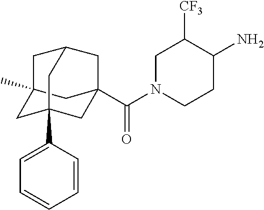 [SMILES: C[C@]12CC3CC(C(=O)N4CCC(N)C(C(F)(F)F)C4)(C1)C[C@@](c1ccccc1)(C3)C2]

The title compound was prepared from (1 S,3R,5R,7S)-3-methyl-5-phenyladamantane-1-carboxylic acid and tert-butyl (3-(trifluoromethyl)piperidin-4-yl)carbamate using the procedure described above for tert-butyl ((S)-3,3-dimethyl-1-((1 S,3R,5R,7S)-3-methyl-5-phenyladamantane-1-carbonyl)piperidin-4-yl)carbamate, followed by cleavage of the Boc-group under acidic conditions. LC/MS m/z: 421.34 [M+H]³⁰

### Example E6: rac-trans-N-(4-aminocyclohexyl)-3-((methylthio)methyl)-5-phenyladamantane-1-carboxamide

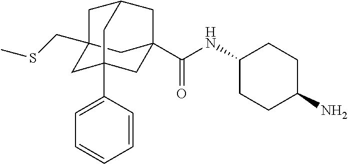 [SMILES: CSCC12CC3CC(C(=O)N[C@H]4CC[C@H](N)CC4)(C1)CC(c1ccccc1)(C3)C2]

### Step 1: tert-butyl ((trans)-4-(rac)-3-((methylthio)methyl)-5-phenyladamantane-1-carboxamido)cyclohexyl)carbamate

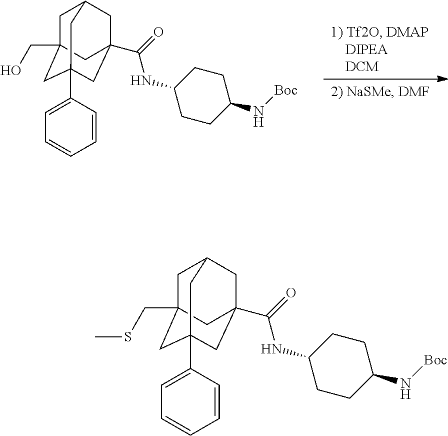

To a solution of tert-butyl trans-4-rac-3-(hydroxymethyl)-5-phenyladamantane-1-carboxamido)cyclohexyl)carbamate (100 mg, 0.21 mmol) in dry DCM (2 mL) at 0° C. is added 4-dimethylaminopyridine (2 mg, catalytic), and diisopropylethylamine (45 uL, 0.25 mmol). Triflic anhydride (42 uL, 0.25 mmol) is added dropwise as a solution in 1 mL DCM and the resulting solution is allowed to warm to room temperature over 2 hours, at which time the solution is washed once with saturated aqueous sodium bicarbonate and the organic layer evaporated. The crude triflate is then dissolved in DMF and sodium methanethiolate (30 mg, 0.42 mmol) is added and the mixture heated to 90° C. overnight. The solution is then diluted with ethyl acetate, washed with water and brine, and evaporated to afford the crude product, which is purified by preparative-HPLC to afford the title compound (28 mg) as a clear oil. LC/MS m/z: 513.33 (M+H)⁺

### Step 2: (rac)-N-((trans)-4-aminocyclohexyl)-3-((methylthio)methyl)-5-phenyladamantane-1-carboxamide

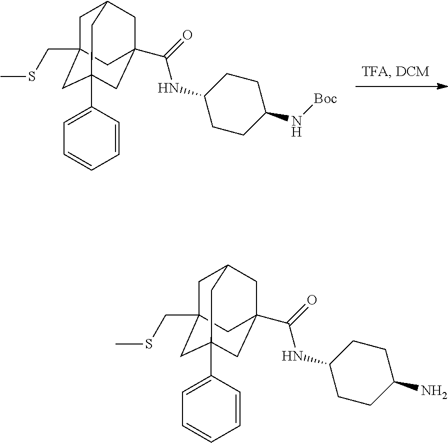

The title compound was prepared using the same Boc-cleavage conditions described for the synthesis of ((S)-4-amino-3,3-dimethylpiperidin-1-yl)((1 S,3R,5R,7S)-3-methyl-5-phenyladamantan-1-yl)methanethione above. LC/MS m/z: 413.34

### Example E7: rac-trans-N-(4-aminocyclohexyl)-3-phenyl-5-((phenylthio)methyl)adamantane-1-carboxamide

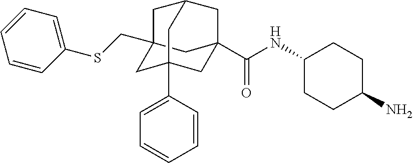 [SMILES: N[C@H]1CC[C@H](NC(=O)C23CC4CC(CSc5ccccc5)(C2)CC(c2ccccc2)(C4)C3)CC1]

The title compound was prepared in the same manner as described above for rac-trans-N-(4-aminocyclohexyl)-3-((methylthio)methyl)-5-phenyladamantane-1-carboxamide (Example E6). LC/MS m/z: 475.43 (M+H)⁺

### Example E8: rac-((S)-4-amino-3,3-dimethylpiperidin-1-yl)(3-phenyl-5-((phenylthio)methyl)adamantan-1-yl)methanone

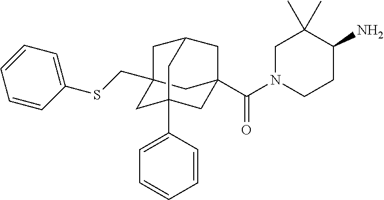 [SMILES: CC1(C)CN(C(=O)C23CC4CC(CSc5ccccc5)(C2)CC(c2ccccc2)(C4)C3)CC[C@@H]1N]

The title compound was prepared in the same manner as described above for rac-trans-N-(4-aminocyclohexyl)-3-((methylthio)methyl)-5-phenyladamantane-1-carboxamide (Example E6). LC/MS m/z: 489.37 (M+H)⁺

Examples F1 to F5 are mixtures of stereoisomers, which were not resolved and were tested as mixtures of isomers.

### Example F1: trans-N-(4-aminocyclohexyl)-6-methyl-3-phenyladamantane-1-carboxamide

### Step 1: tert-butyl (trans-4-(6-methylene-3-phenyladamantane-1-carboxamido)cyclohexyl)carbamate

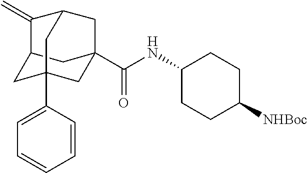 [SMILES: C=C1C2CC3(C(=O)N[C@H]4CC[C@H](NC(=O)OC(C)(C)C)CC4)CC1CC(c1ccccc1)(C2)C3]

The title compound was prepared from 6-methylene-3-phenyladamantane-1-carboxylic acid and trans-tert-butyl N-(4-aminocyclohexyl)carbamate in the same manner as described above for tert-butyl ((S)-3,3-dimethyl-1-((1 S,3R,5R,7S)-3-methyl-5-phenyladamantane-1-carbonyl)piperidin-4-yl)carbamate. LC/MS m/z: 465.48 [M+H]⁺, 506.37 [2M+H]⁺

### Step 2: trans-N-(4-aminocyclohexyl)-6-methyl-3-phenyladamantane-1-carboxamide

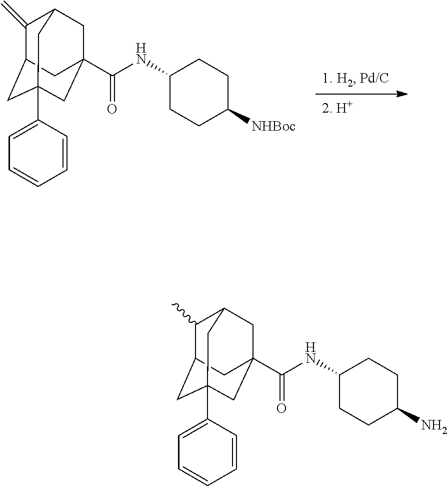 [SMILES: C=C1C2CC3(C(=O)N[C@H]4CC[C@H](NC(=O)OC(C)(C)C)CC4)CC1CC(c1ccccc1)(C2)C3 ; "2. H+" ; "1. H2, Pd/C" ; CC1C2CC3(C(=O)N[C@H]4CC[C@H](N)CC4)CC1CC(c1ccccc1)(C2)C3]

To a nitrogen flushed flask is added 10% Pd/C (20 mg) and a solution of the starting material (40 mg) in methanol (3 mL). The flask is then flushed with hydrogen and stirred under a hydrogen atmosphere overnight. The mixture is then filtered through celite and washed with methanol. Evaporation of the filtrate gives 36 mg of the boc-amine, which is dissolved in DCM (2 mL) and TFA (1 mL) is added. After stirring for 3 hours the solution is evaporated and the residue purified by prep-HPLC, giving 23 mg of the title compound as a white solid. LC/MS m/z: 367.38 [M+H]⁺, 408.35 [M+H+CH₃CN]⁺

Examples F2-F5 were prepared in the same manner as described above for trans-N-(4-aminocyclohexyl)-6-methyl-3-phenyladamantane-1-carboxamide (Example F1, steps 1 and 2) using appropriate carboxylic acid and amine as starting materials.

| Ex. | Starting materials | Product/Name | Analytical Data |
|---|---|---|---|
| F2 | trans-tert-butyl N-(4- aminocyclohexyl) carbamate 6-ethylidene-3- phenyladamantane-1- carboxylic acid | 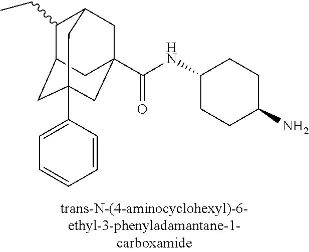 [SMILES: CCC1C2CC3(C(=O)N[C@H]4CC[C@H](N)CC4)CC1CC(c1ccccc1)(C2)C3] | LC/MS m/z: 381.44 [M + H]⁺ |
| F3 | trans-tert-butyl N-(4- aminocyclohexyl) carbamate 3-phenyl-6- propylideneadamantane- 1-carboxylic acid | 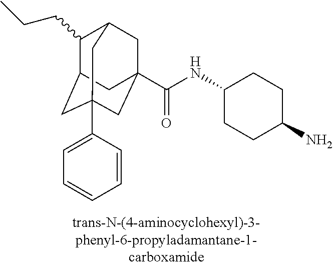 [SMILES: CCCC1C2CC3(C(=O)N[C@H]4CC[C@H](N)CC4)CC1CC(c1ccccc1)(C2)C3] | LC/MS m/z: 395.45 [M + H]⁺, 436.36 [M + H + CH₃CN]⁺ |
| F4 | trans-tert-butyl N-(4- aminocyclohexyl) carbamate 6- (cyclopropylmethylene)- 3-phenyladamantane- 1-carboxylic acid | 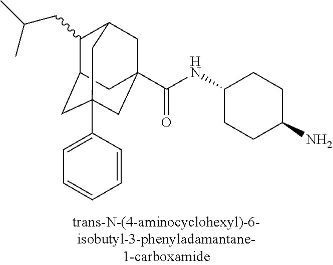 [SMILES: CC(C)CC1C2CC3(C(=O)N[C@H]4CC[C@H](N)CC4)CC1CC(c1ccccc1)(C2)C3] | LC/MS m/z: 409.46 [M + H]⁺, 450.47 [M + H + CH₃CN]⁺ |
| F5 | tert-butyl (S)-(3,3- dimethylpiperidin-4- yl)carbamate 6-ethylidene-3- phenyladamantane-1- carboxylic acid | 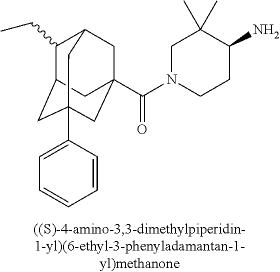 [SMILES: CCC1C2CC3(C(=O)N4CC[C@H](N)C(C)(C)C4)CC1CC(c1ccccc1)(C2)C3] | LC/MS m/z: 395.41 [M + H]⁺ ¹H NMR (300 MHz, DMSO-d₆) HCl salt: d 8.02 (brs, 3H), 7.38- 7.40 (m, 2H), 7.30-7.33 (m, 2H), 7.18 (t, 1H), 4.31-4.40 (m, 1H), 4.02-4.09 (m, 1H), 3.03-3.11 (m, 2H), 2.54 (d, 1H), 1.94-2.03 (m, 7H), 1.78-1.89 (m, 3H), 1.70- 1.77 (m, 1H), 1.55-1.60 (m, 1H), 1.40-1.49 (m, 2H), 1.04 (d, 1H), 1.01 (s, 3H), 0.87-0.91 (m, 3H), 0.78 (s, 3H). |

Examples G to G6 and G8 are racemic mixtures and Examples G7 and G9-G12 are mixtures of diastereomers, which were not resolved and were tested as mixtures of isomers.

Examples G1-G12 were prepared in the same manner as described above for tert-butyl ((S)-3,3-dimethyl-1-((1 S,3R,5R,7S)-3-methyl-5-phenyladamantane-1-carbonyl)piperidin-4-yl)carbamate using appropriate carboxylic acid and amine as starting materials, followed by cleavage of the Boc-group under acidic conditions.

| Ex. | Starting materials | Product/Name | Analytical Data |
|---|---|---|---|
| G1 | trans-tert-butyl N-(4- aminocyclohexyl) carbamate 6-ethylidene-3- phenyladamantane-1- carboxylic acid | 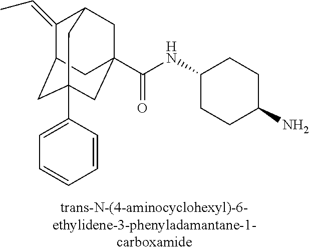 [SMILES: CC=C1C2CC3(C(=O)N[C@H]4CC[C@H](N)CC4)CC1CC(c1ccccc1)(C2)C3] | LC/MS m/z: 379.41 [M + H]⁺ |
| G2 | trans-tert-butyl N-(4- aminocyclohexyl) carbamate 3-phenyl-6- propylideneadamantane- 1-carboxylic acid | 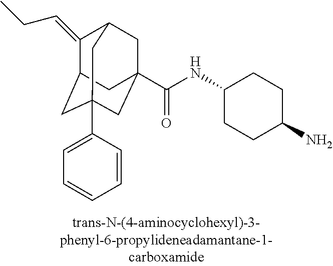 [SMILES: CCC=C1C2CC3(C(=O)N[C@H]4CC[C@H](N)CC4)CC1CC(c1ccccc1)(C2)C3] | LC/MS m/z: 393.43 [M + H]⁺, 434.36 [M + H + CH₃CN]⁺ |
| G3 | trans-tert-butyl N-(4- aminocyclohexyl) carbamate 6- (cyclopropylmethylene)- 3-phenyladamantane- 1-carboxylic acid | 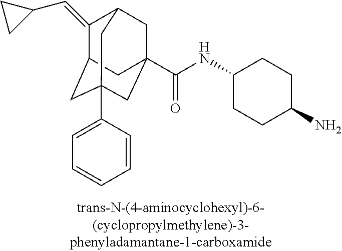 [SMILES: N[C@H]1CC[C@H](NC(=O)C23CC4CC(c5ccccc5)(CC(C2)C4=CC2CC2)C3)CC1] | LC/MS m/z: 405.43 [M + H]⁺, 446.39 [M + H + CH₃CN]⁺ |
| G4 | trans-tert-butyl N-(4- aminocyclohexyl) carbamate 6-(benzylidene)-3- phenyladamantane-1- carboxylic acid | 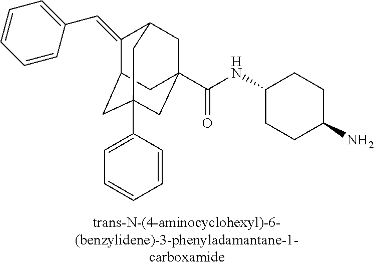 [SMILES: N[C@H]1CC[C@H](NC(=O)C23CC4CC(c5ccccc5)(CC(C2)C4=Cc2ccccc2)C3)CC1] | LC/MS m/z: 441.37 [M + H]⁺ |
| G5 | tert-butyl piperidin-4- ylcarbamate 3-phenyl-6- propylideneadamantane- 1-carboxylic acid | 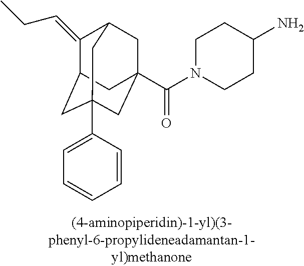 [SMILES: CCC=C1C2CC3(C(=O)N4CCC(N)CC4)CC1CC(c1ccccc1)(C2)C3] | LC/MS m/z: 379.41 [M + H]⁺ |
| G6 | trans-tert-butyl N-(4- aminocyclohexyl) carbamate 6-(3-methylbutylidene)- 3-phenyladamantane- 1-carboxylic acid | 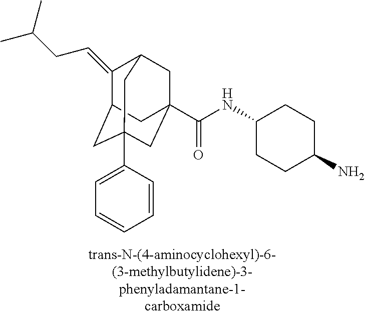 [SMILES: CC(C)CC=C1C2CC3(C(=O)N[C@H]4CC[C@H](N)CC4)CC1CC(c1ccccc1)(C2)C3] | LC/MS m/z: 421.38 [M + H]⁺ |
| G7 | tert-butyl (S)-(3,3- dimethylpiperidin-4- yl)carbamate 3-phenyl-6- propylideneadamantane- 1-carboxylic acid | 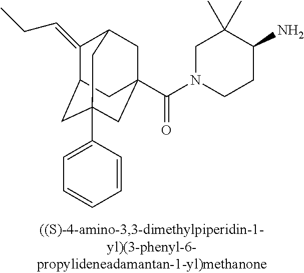 [SMILES: CCC=C1C2CC3(C(=O)N4CC[C@H](N)C(C)(C)C4)CC1CC(c1ccccc1)(C2)C3] | LC/MS m/z: 407.43 [M + H]⁺ |
| G8 | trans-tert-butyl N-(4- aminocyclohexyl) carbamate 6-(3-methylbut-2-en-1- ylidene)-3- phenyladamantane-1- carboxylic acid | 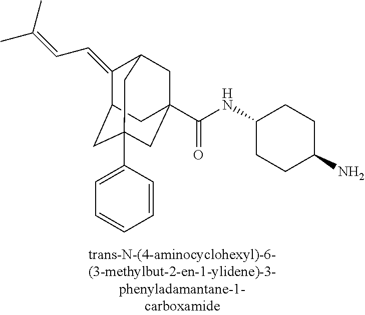 | LC/MS m/z: 460.58 [M + H + CH₃CN]⁺ |
| G9 | tert-butyl (S)-(3,3- dimethylpiperidin-4- yl)carbamate 6-butylidene-3- phenyladamantane-1- carboxylic acid | 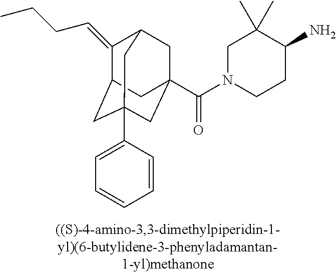 [SMILES: CCCC=C1C2CC3(C(=O)N4CC[C@H](N)C(C)(C)C4)CC1CC(c1ccccc1)(C2)C3] | LC/MS m/z: 421.44 [M + H]⁺ |
| G10 | tert-butyl (S)-(3,3- dimethylpiperidin-4- yl)carbamate 6-allylidene-3- phenyladamantane-1- carboxylic acid | 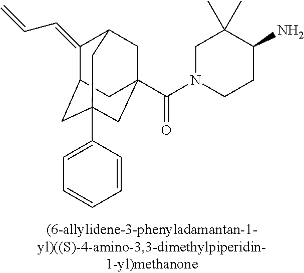 [SMILES: C=CC=C1C2CC3(C(=O)N4CC[C@H](N)C(C)(C)C4)CC1CC(c1ccccc1)(C2)C3] | LC/MS m/z: 405.35 [M + H]⁺ |
| G11 | tert-butyl (S)-(3,3- dimethylpiperidin-4- yl) carbamate 6-ethylidene-3- phenyladamantane-1- carboxylic acid | 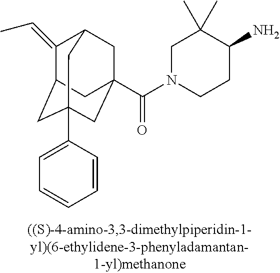 [SMILES: CC=C1C2CC3(C(=O)N4CC[C@H](N)C(C)(C)C4)CC1CC(c1ccccc1)(C2)C3] | LC/MS m/z: 393.45 [M + H]⁺ |
| G12 | tert-butyl (S)-(3,3- dimethylpiperidin-4- yl)carbamate 3-phenyl-6-(2- phenylethylidene)adam antane-1-carboxylic acid | 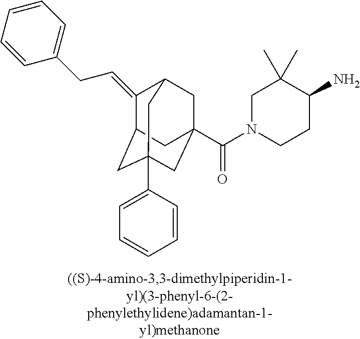 [SMILES: CC1(C)CN(C(=O)C23CC4CC(c5ccccc5)(CC(C2)C4=CCc2ccccc2)C3)CC[C@@H]1N] | LC/MS m/z: 469.40 [M + H]⁺ |
| G13 | tert-butyl (4- methylpiperidin-4- yl)carbamate 3-phenyl-6- propylideneadamantane- 1-carboxylic acid | 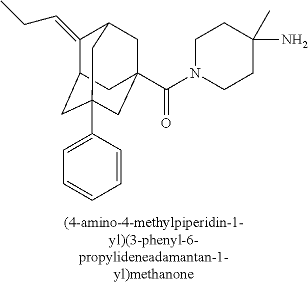 [SMILES: CCC=C1C2CC3(C(=O)N4CCC(C)(N)CC4)CC1CC(c1ccccc1)(C2)C3] | LC/MS m/z: 393.3 [M + H]⁺ |
| G14 | (S)-tert-butyl piperidin- 3-ylcarbamate 3-phenyl-6- propylideneadamantane- 1-carboxylic acid | 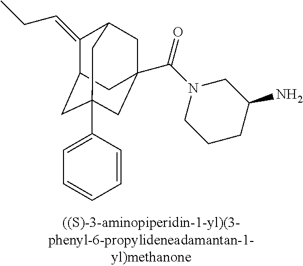 [SMILES: CCC=C1C2CC3(C(=O)N4CCC[C@H](N)C4)CC1CC(c1ccccc1)(C2)C3] | LC/MS m/z: 379.3 [M + H]⁺ |
| G15 | (R)-tert-butyl piperidin- 3-ylcarbamate 3-phenyl-6- propylideneadamantane- 1-carboxylic acid | 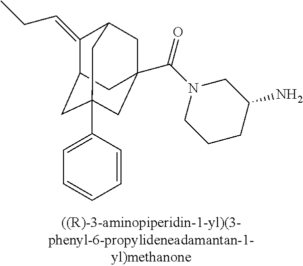 [SMILES: CCC=C1C2CC3(C(=O)N4CCC[C@@H](N)C4)CC1CC(c1ccccc1)(C2)C3] | LC/MS m/z: 379.3 [M + H]⁺ |
| G16 | trans-tert-butyl N-[(4- aminocyclohexyl)methyl] carbamate 3-phenyl-6- propylideneadamantane- 1-carboxylic acid | 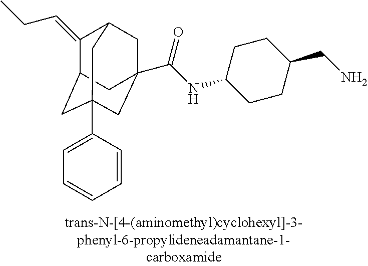 [SMILES: CCC=C1C2CC3(C(=O)N[C@H]4CC[C@H](CN)CC4)CC1CC(c1ccccc1)(C2)C3] | LC/MS m/z: 407.3 [M + H]⁺ |
| G17 | tert-butyl 2,7- diazaspiro[3.5]nonane- 2-carboxylate 3-phenyl-6- propylideneadamantane- 1-carboxylic acid | 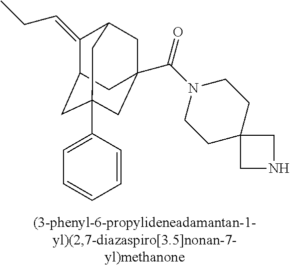 [SMILES: CCC=C1C2CC3(C(=O)N4CCC5(CC4)CNC5)CC1CC(c1ccccc1)(C2)C3] | LC/MS m/z: 405.3 [M + H]⁺ |
| G18 | trans-tert-butyl N-[(3- aminocyclobutyl)methyl] carbamate 3-phenyl-6- propylideneadamantane- 1-carboxylic acid | 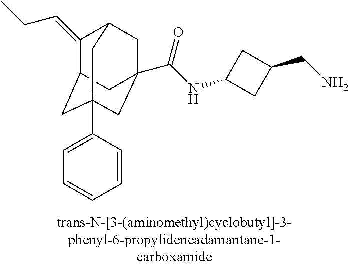 [SMILES: CCC=C1C2CC3(C(=O)N[C@H]4C[C@H](CN)C4)CC1CC(c1ccccc1)(C2)C3] | LC/MS m/z: 379.3 [M + H]⁺ |
| G19 | tert-butyl (2-(piperidin- 4-yl)ethyl)carbamate 3-phenyl-6- propylideneadamantane- 1-carboxylic acid | 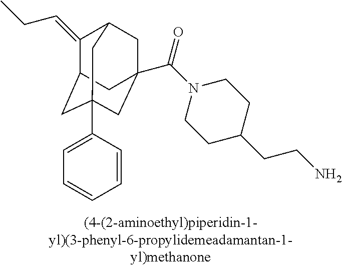 [SMILES: CCC=C1C2CC3(C(=O)N4CCC(CCN)CC4)CC1CC(c1ccccc1)(C2)C3] | LC/MS m/z: 407.3 [M + H]⁺ |

### Example H1: trans-N-(4-aminocyclohexyl)-6-hydroxy-6-methyl-3-phenyladamantane-1-carboxamide (Unknown Isomer)

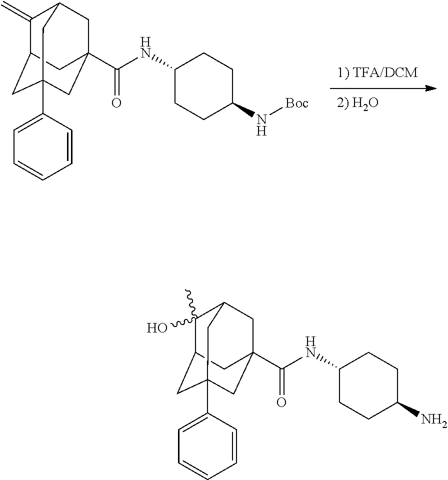

20 mg of starting material is dissolved in 1 mL DCM and 0.5 mL TFA is added. The mixture is stirred overnight, at which time LC/MS indicates the starting material was converted to the TFA ester. The volatiles were evaporated and the residue purified by prep-HPLC using water/acetonitrile as eluents. Two products are isolated during prep-hplc purification and LC/MS shows these products to be the two isomers of the title compound, hydrolysis products of the TFA ester. LC/MS m/z: 383.38 [M+H]⁺, 429.35 [M+H+CH₃CN]³⁰

### Example H2: trans-N-(4-aminocyclohexyl)-6-hydroxy-6-methyl-3-phenyladamantane-1-carboxamide (Unknown Isomer)

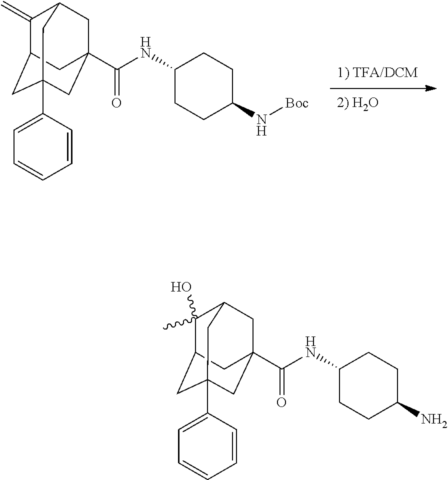

The second isomer from the procedure above was isolated during the same prep-HPLC run as the previous compound. LC/MS m/z: 383.38 [M+H]⁺, 429.35 [M+H+CH₃CN]⁺

### Example H3: ((S)-4-amino-3,3-dimethylpiperidin-1-yl)(6-chloro-6-ethyl-3-phenyladamantan-1-yl)methanone

### Step 1: tert-butyl ((S)-1-(6-ethylidene-3-phenyladamantane-1-carbonyl)-3,3-dimethylpiperidin-4-yl)carbamate

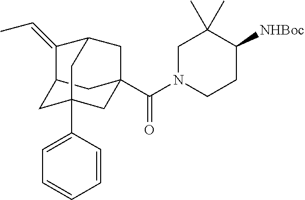 [SMILES: CC=C1C2CC3(C(=O)N4CC[C@H](NC(=O)OC(C)(C)C)C(C)(C)C4)CC1CC(c1ccccc1)(C2)C3]

The title compound was prepared from 6-ethylidene-3-phenyladamantane-1-carboxylic acid and tert-butyl (S)-(3,3-dimethylpiperidin-4-yl)carbamate in the same manner as described above for tert-butyl ((S)-3,3-dimethyl-1-((1 S,3R,5R,7S)-3-methyl-5-phenyladamantane-1-carbonyl)piperidin-4-yl)carbamate. LC/MS m/z: 493.48 [M+H]³⁰

### Step 2: ((S)-4-amino-3,3-dimethylpiperidin-1-yl)(6-chloro-6-ethyl-3-phenyladamantan-1-yl)methanone

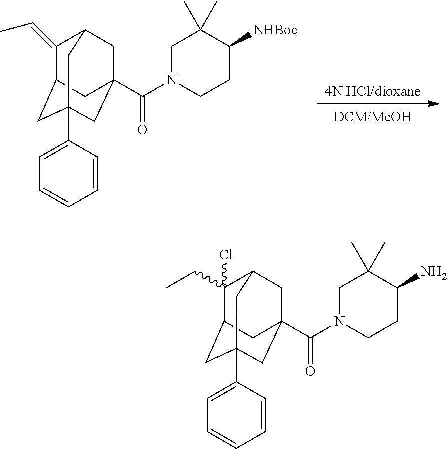

To a solution of tert-butyl ((S)-1-(6-ethylidene-3-phenyladamantane-1-carbonyl)-3,3-dimethylpiperidin-4-yl) carbamate (0.098 g, 0.2 mmol) in DCM (4 mL) was added methanol (0.4) followed by 4N HCl/1,4-dioxane (0.2 mL). Resulting reaction mixture was stirred at RT overnight. Solvent was evaporated under vacuum to give inseparable mixture of title compound (77%) along with ((S)-4-amino-3,3-dimethylpiperidin-1-yl) (6-ethylidene-3-phenyladamantan-1-yl) methanone (23%). Yield: 0.056 g. LC/MS m/z: 429.4 [M]⁺

### Example H4: trans-N-(4-aminocyclohexyl)-6-(2-fluoroethyl)-3-phenyladamantane-1-carboxamide

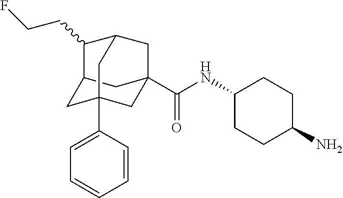 [SMILES: N[C@H]1CC[C@H](NC(=O)C23CC4CC(c5ccccc5)(CC(C2)C4CCF)C3)CC1]

The title compound was prepared from 6-(2-fluoroethyl)-3-phenyladamantane-1-carboxylic acid and trans-tert-butyl N-(4-aminocyclohexyl)carbamate in the same manner as described above for tert-butyl ((S)-3,3-dimethyl-1-((1 S,3R,5R,7S)-3-methyl-5-phenyladamantane-1-carbonyl)piperidin-4-yl)carbamate (Example A1, step 1), followed by cleavage of the Boc-group under acidic conditions. LC/MS m/z: 399.3 [M+H]⁺

### Example H5: trans-N-(4-aminocyclohexyl)-6-(2-chloroethyl)-3-phenyladamantane-1-carboxamide

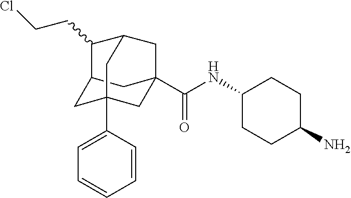 [SMILES: N[C@H]1CC[C@H](NC(=O)C23CC4CC(c5ccccc5)(CC(C2)C4CCCl)C3)CC1]

The title compound was prepared from 6-(2-chloroethyl)-3-phenyladamantane-1-carboxylic acid and trans-tert-butyl N-(4-aminocyclohexyl)carbamate in the same manner as described above for tert-butyl ((S)-3,3-dimethyl-1-((1 S,3R,5R,7S)-3-methyl-5-phenyladamantane-1-carbonyl)piperidin-4-yl)carbamate (Example A1, step 1), followed by cleavage of the Boc-group under acidic conditions. LC/MS m/z: 415.3 [M+H]⁺

### Example H-6: trans-N-(4-aminocyclohexyl)-6-(2,2-difluoroethyl)-3-phenyladamantane-1-carboxamide

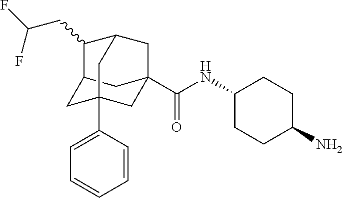 [SMILES: N[C@H]1CC[C@H](NC(=O)C23CC4CC(c5ccccc5)(CC(C2)C4CC(F)F)C3)CC1]

The title compound was prepared from 6-(2,2-difluoroethyl)-3-phenyladamantane-1-carboxylic acid and trans-tert-butyl N-(4-aminocyclohexyl)carba mate in the same manner as described above for tert-butyl ((S)-3,3-dimethyl-1-((1 S,3R,5R,7S)-3-methyl-5-phenyladamantane-1-carbonyl)piperidin-4-yl)carbamate (Example A1, step 1), followed by cleavage of the Boc-group under acidic conditions. LC/MS m/z: 417.3 [M+H]⁺

Following schemes 1-15 and procedures above using the appropriate starting materials and applying modifications apparent to those skilled in the art, e.g. by making routine modifications of reaction conditions, the following examples can be made:

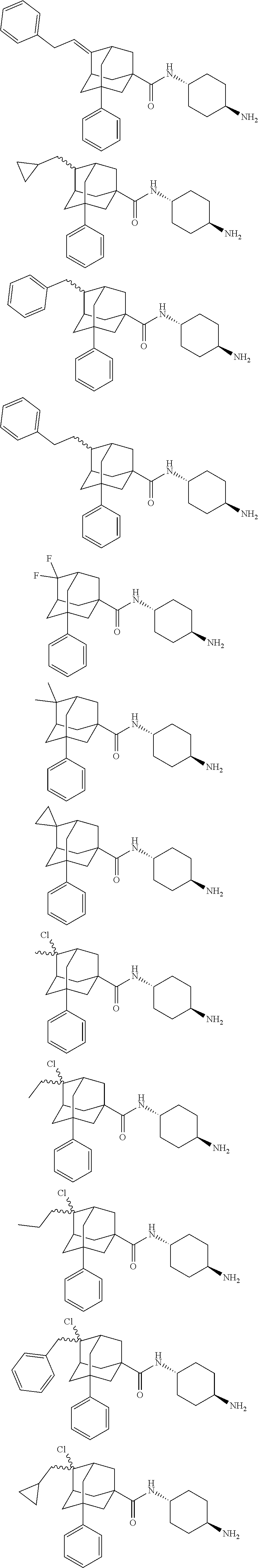

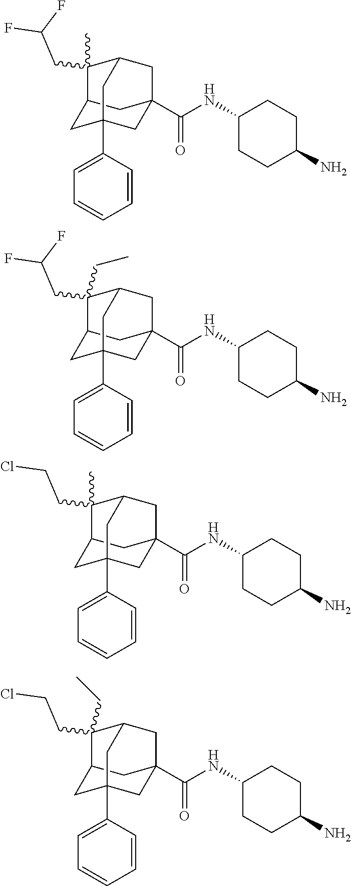

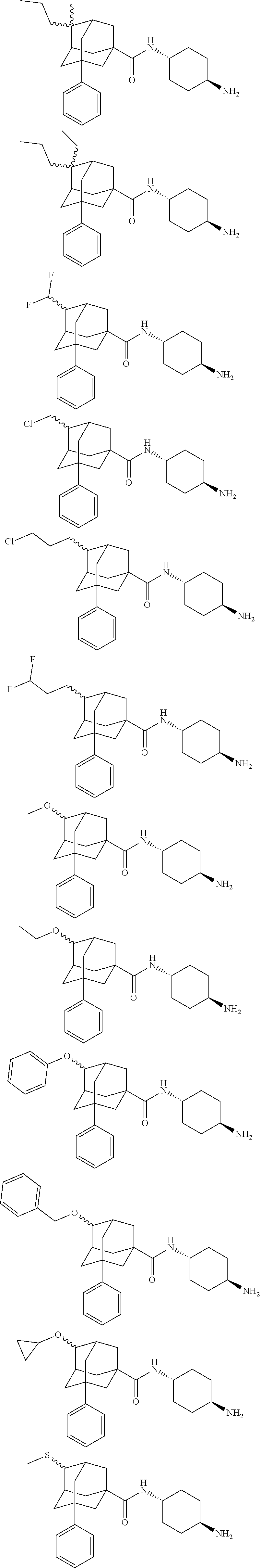

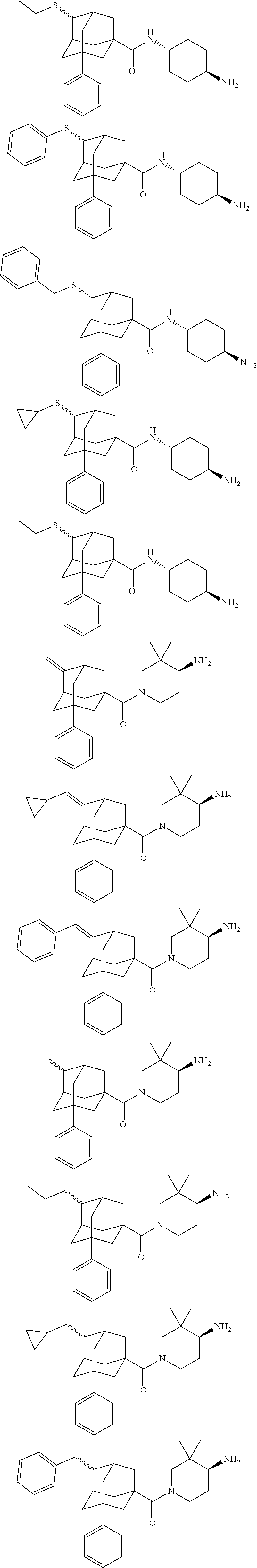

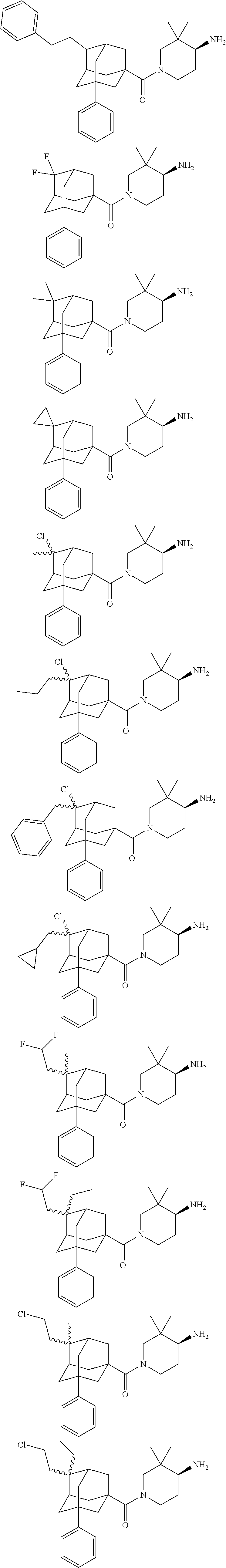

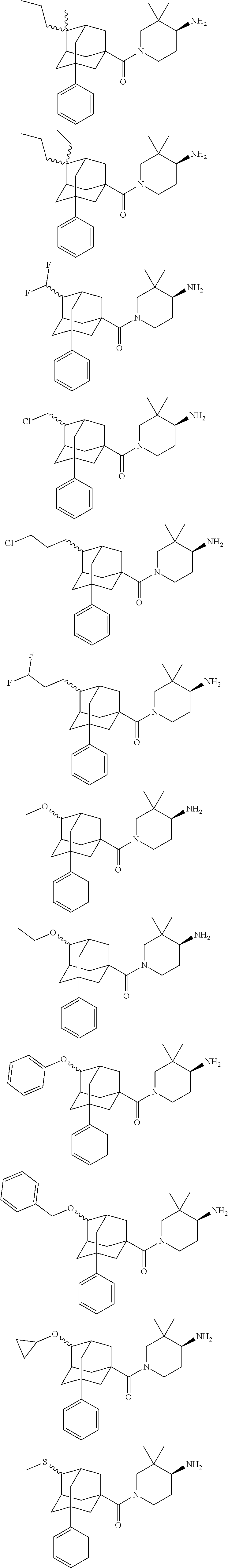

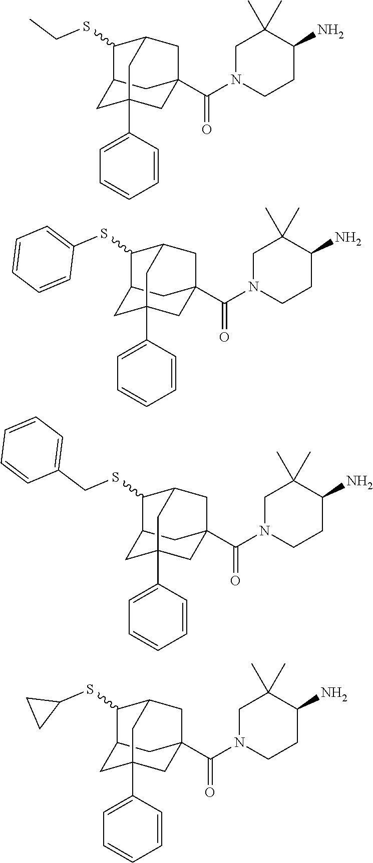

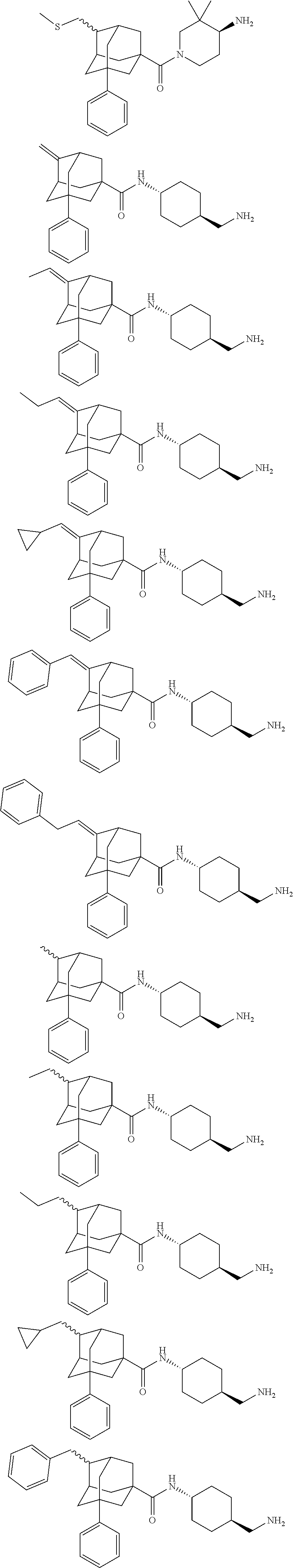

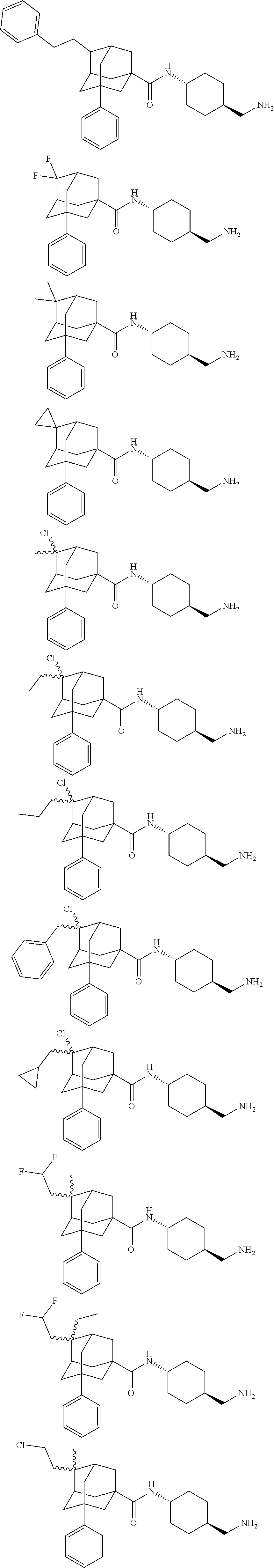

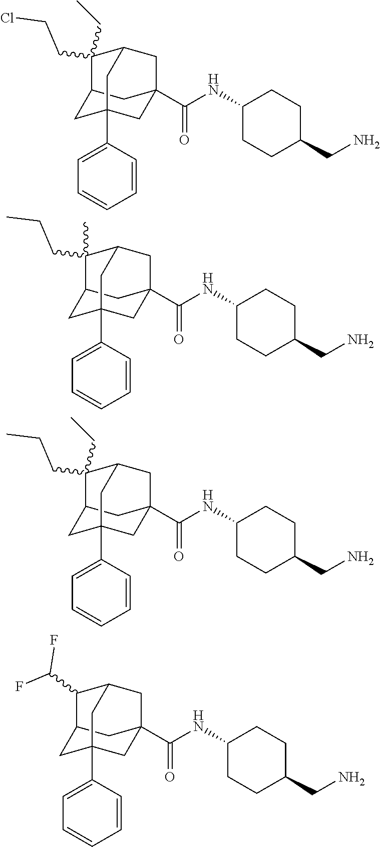

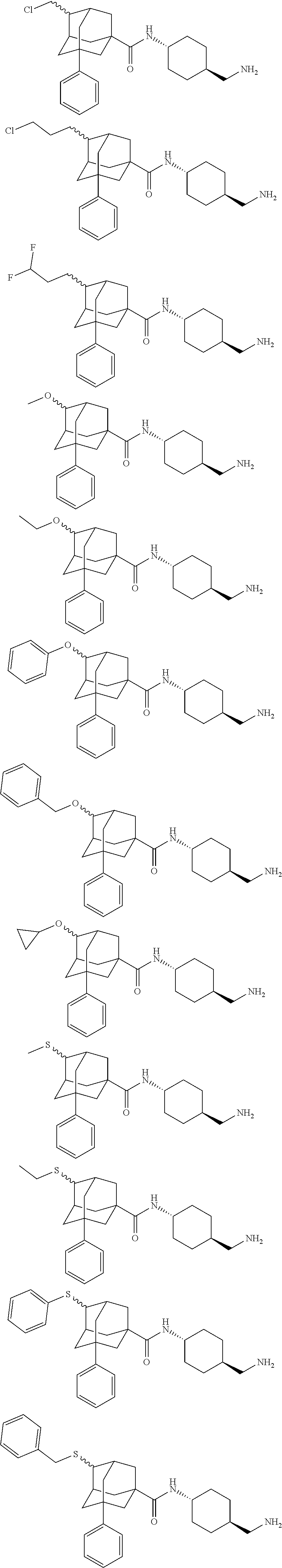

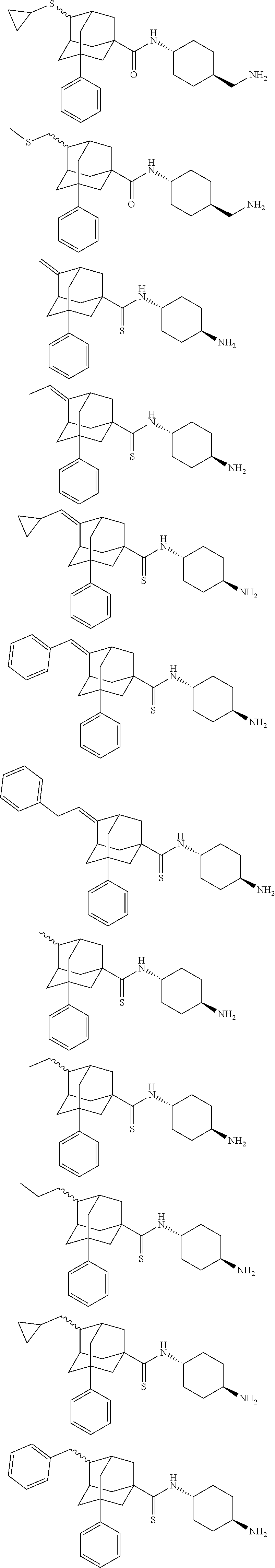

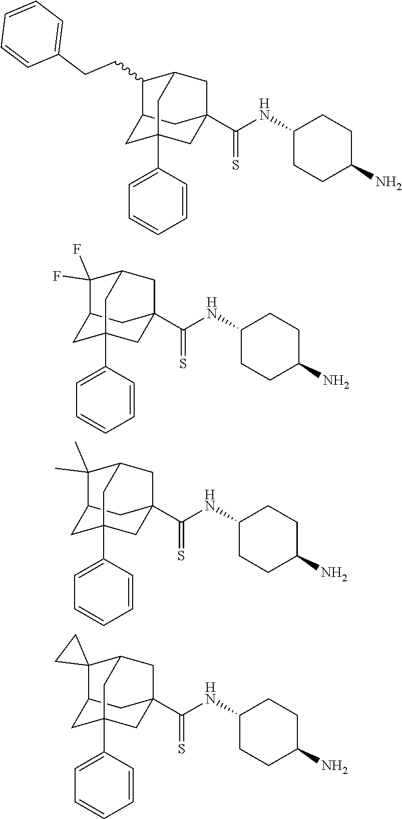

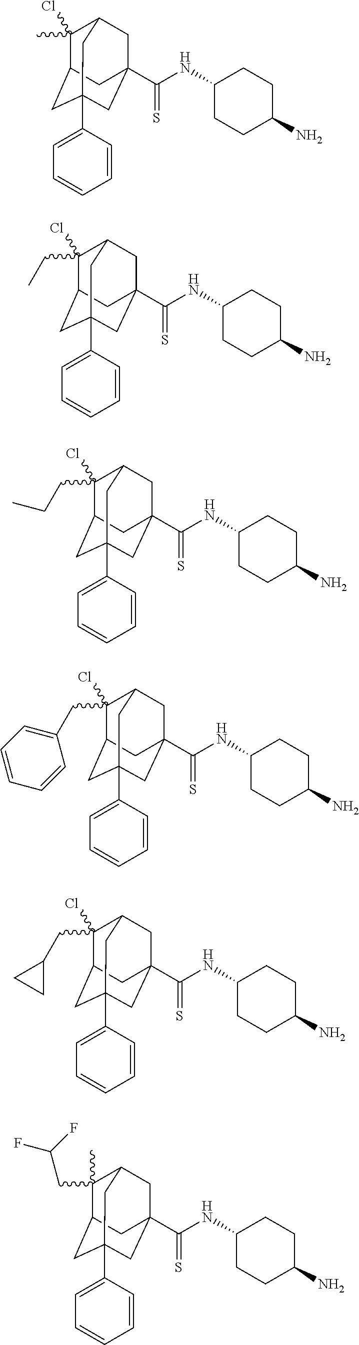

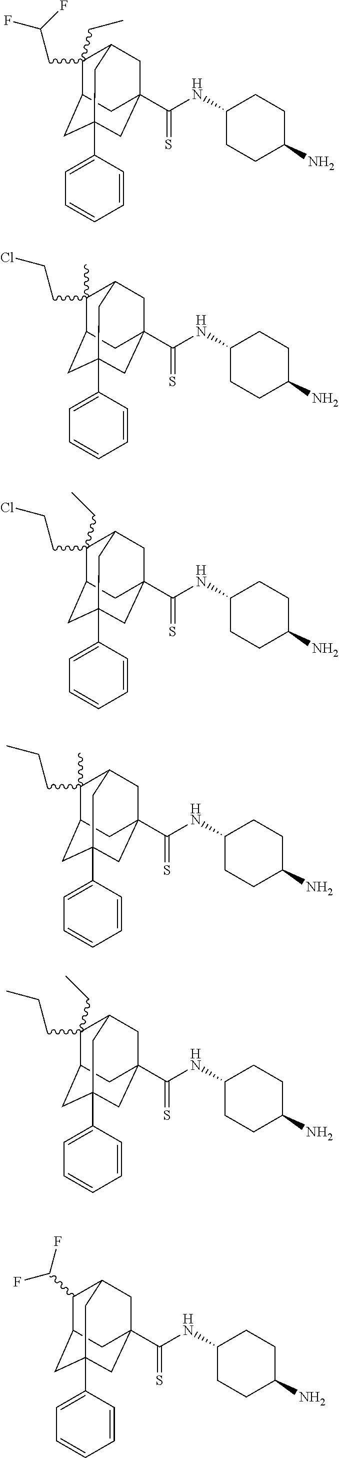

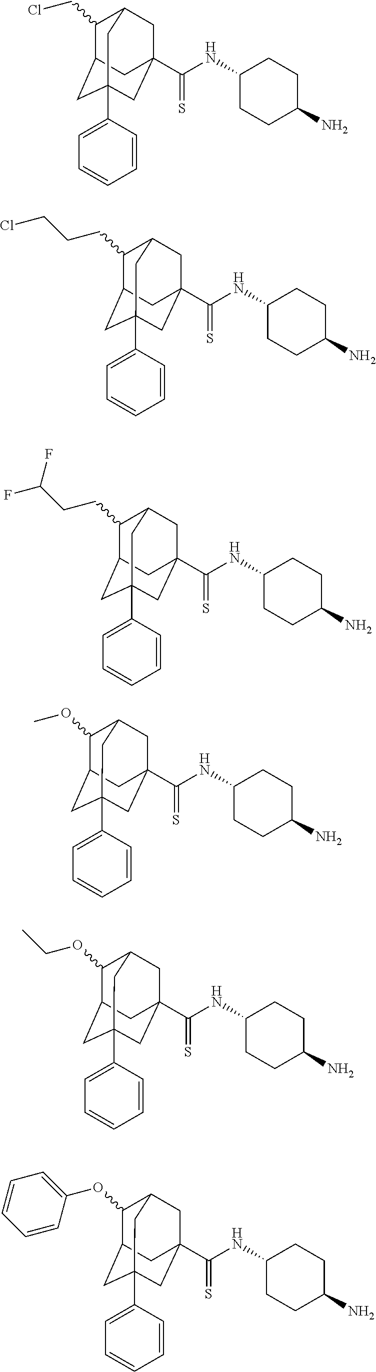

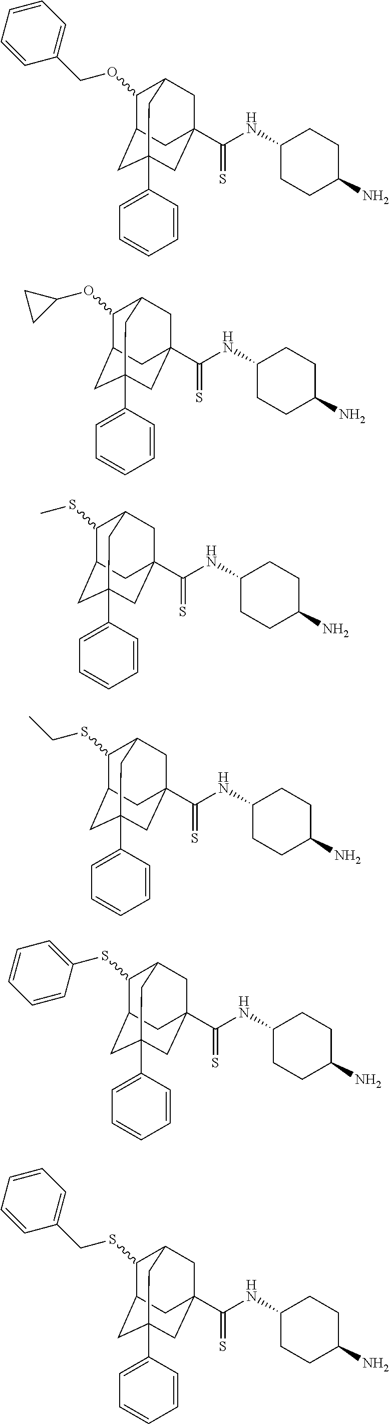

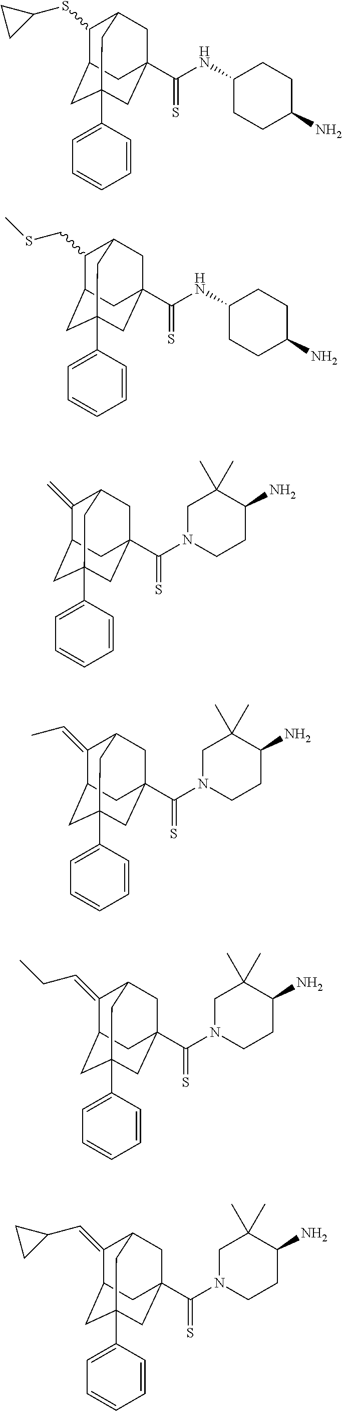 [SMILES: N[C@H]1CC[C@H](NC(=S)C23CC4CC(c5ccccc5)(CC(C2)C4SC2CC2)C3)CC1 ; CSCC1C2CC3(C(=S)N[C@H]4CC[C@H](N)CC4)CC1CC(c1ccccc1)(C2)C3 ; C=C1C2CC3(C(=S)N4CC[C@H](N)C(C)(C)C4)CC1CC(c1ccccc1)(C2)C3 ; CC=C1C2CC3(C(=S)N4CC[C@H](N)C(C)(C)C4)CC1CC(c1ccccc1)(C2)C3 ; CCC=C1C2CC3(C(=S)N4CC[C@H](N)C(C)(C)C4)CC1CC(c1ccccc1)(C2)C3 ; CC1(C)CN(C(=S)C23CC4CC(c5ccccc5)(CC(C2)C4=CC2CC2)C3)CC[C@@H]1N]

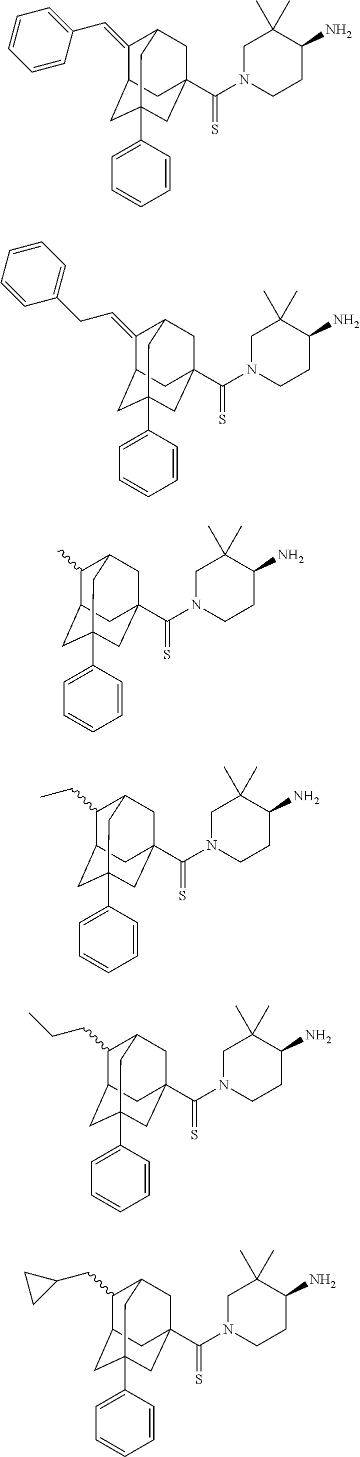 [SMILES: CC1(C)CN(C(=S)C23CC4CC(c5ccccc5)(CC(C2)C4=Cc2ccccc2)C3)CC[C@@H]1N ; CC1(C)CN(C(=S)C23CC4CC(c5ccccc5)(CC(C2)C4=CCc2ccccc2)C3)CC[C@@H]1N ; CC1C2CC3(C(=S)N4CC[C@H](N)C(C)(C)C4)CC1CC(c1ccccc1)(C2)C3 ; CCC1C2CC3(C(=S)N4CC[C@H](N)C(C)(C)C4)CC1CC(c1ccccc1)(C2)C3 ; CCCC1C2CC3(C(=S)N4CC[C@H](N)C(C)(C)C4)CC1CC(c1ccccc1)(C2)C3 ; CC1(C)CN(C(=S)C23CC4CC(c5ccccc5)(CC(C2)C4CC2CC2)C3)CC[C@@H]1N]

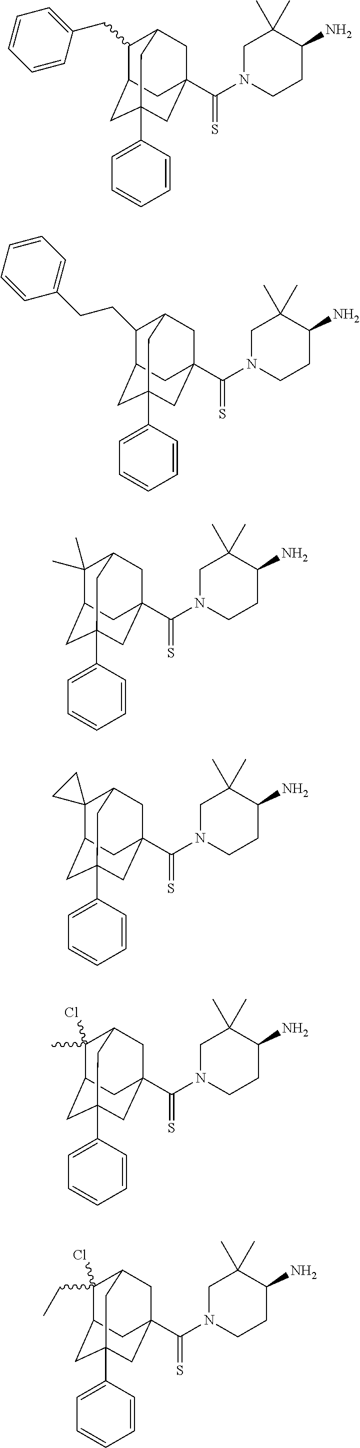

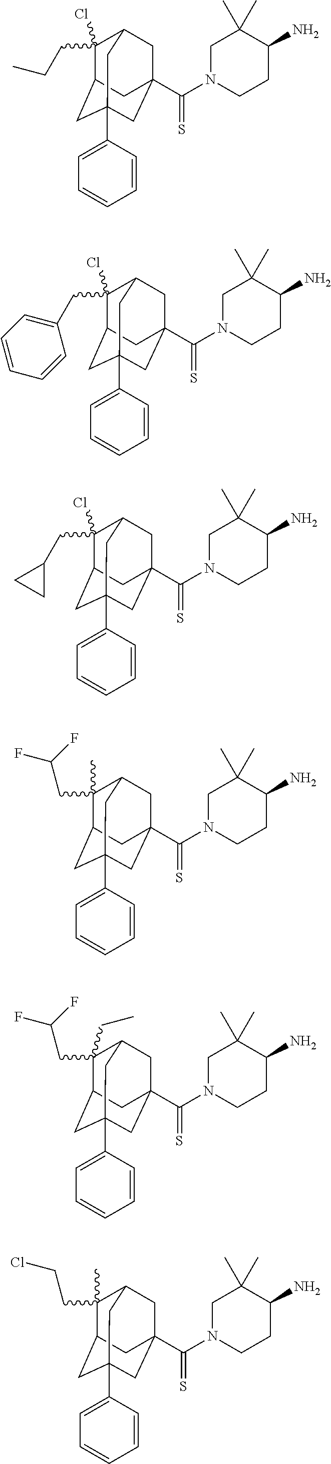

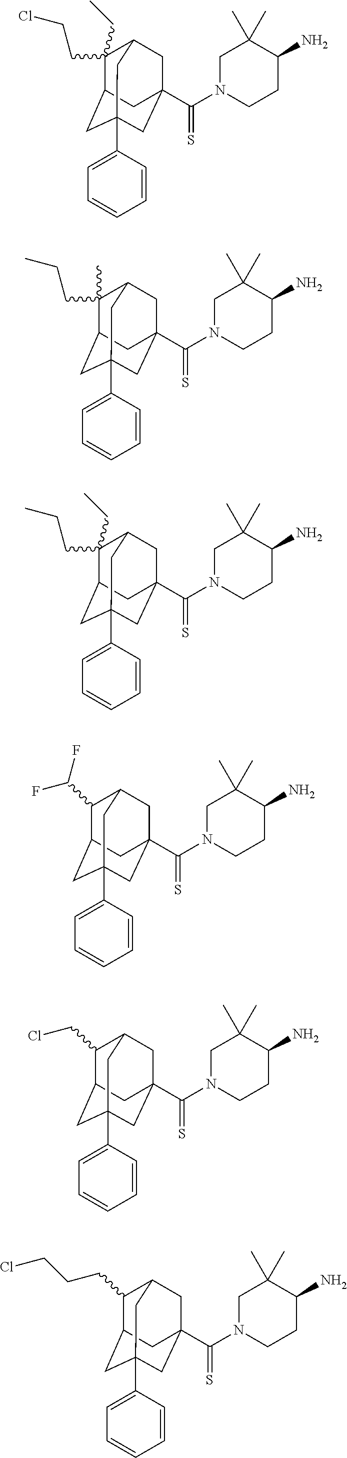

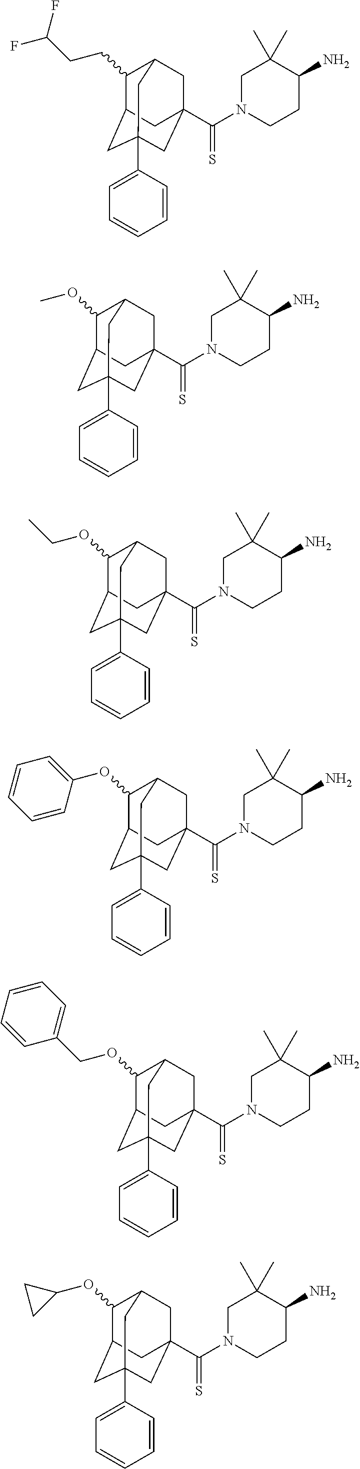 [SMILES: CC1(C)CN(C(=S)C23CC4CC(c5ccccc5)(CC(C2)C4CCC(F)F)C3)CC[C@@H]1N ; COC1C2CC3(C(=S)N4CC[C@H](N)C(C)(C)C4)CC1CC(c1ccccc1)(C2)C3 ; CCOC1C2CC3(C(=S)N4CC[C@H](N)C(C)(C)C4)CC1CC(c1ccccc1)(C2)C3 ; CC1(C)CN(C(=S)C23CC4CC(c5ccccc5)(CC(C2)C4Oc2ccccc2)C3)CC[C@@H]1N ; CC1(C)CN(C(=S)C23CC4CC(c5ccccc5)(CC(C2)C4OCc2ccccc2)C3)CC[C@@H]1N ; CC1(C)CN(C(=S)C23CC4CC(c5ccccc5)(CC(C2)C4OC2CC2)C3)CC[C@@H]1N]

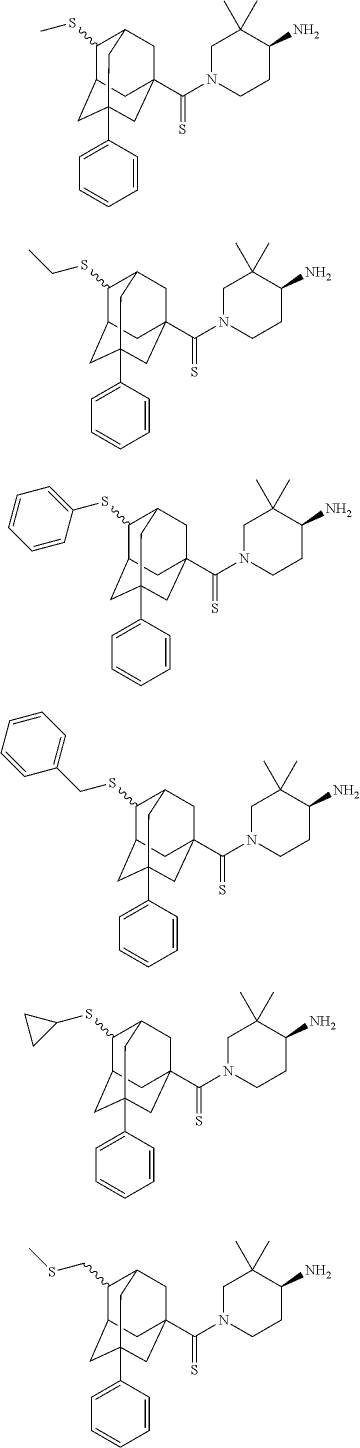 [SMILES: CSC1C2CC3(C(=S)N4CC[C@H](N)C(C)(C)C4)CC1CC(c1ccccc1)(C2)C3 ; CCSC1C2CC3(C(=S)N4CC[C@H](N)C(C)(C)C4)CC1CC(c1ccccc1)(C2)C3 ; CC1(C)CN(C(=S)C23CC4CC(c5ccccc5)(CC(C2)C4Sc2ccccc2)C3)CC[C@@H]1N ; CC1(C)CN(C(=S)C23CC4CC(c5ccccc5)(CC(C2)C4SCc2ccccc2)C3)CC[C@@H]1N ; CC1(C)CN(C(=S)C23CC4CC(c5ccccc5)(CC(C2)C4SC2CC2)C3)CC[C@@H]1N ; CSCC1C2CC3(C(=S)N4CC[C@H](N)C(C)(C)C4)CC1CC(c1ccccc1)(C2)C3]

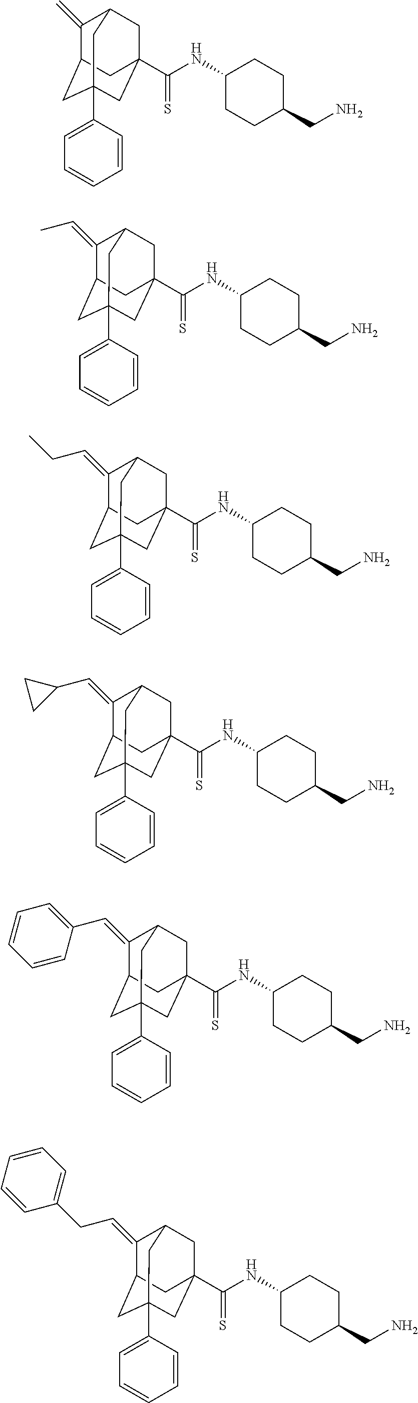

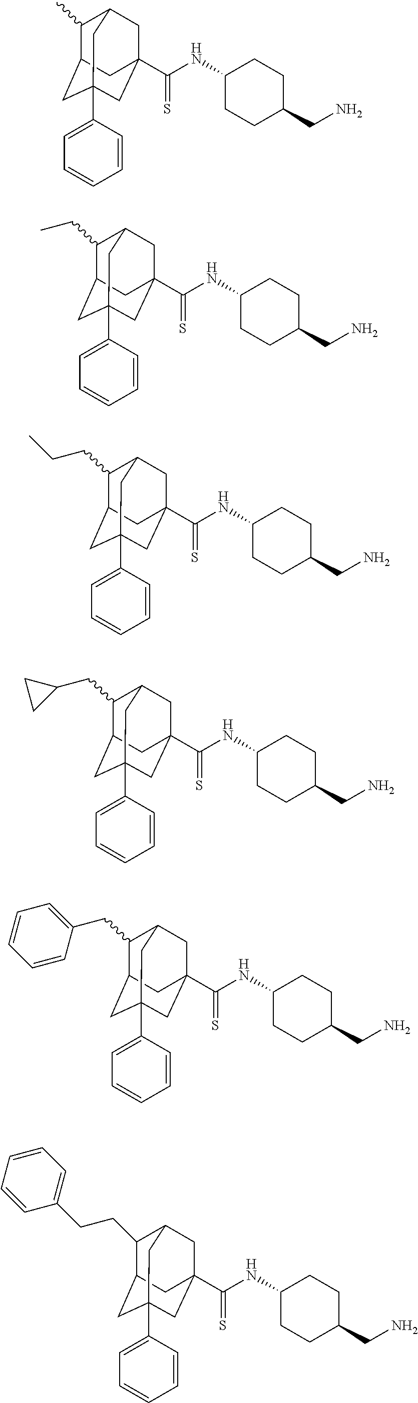

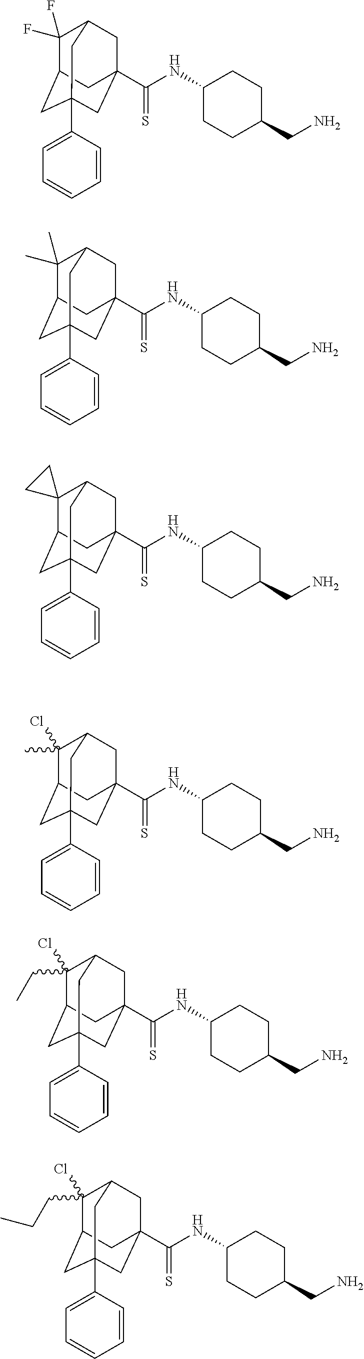

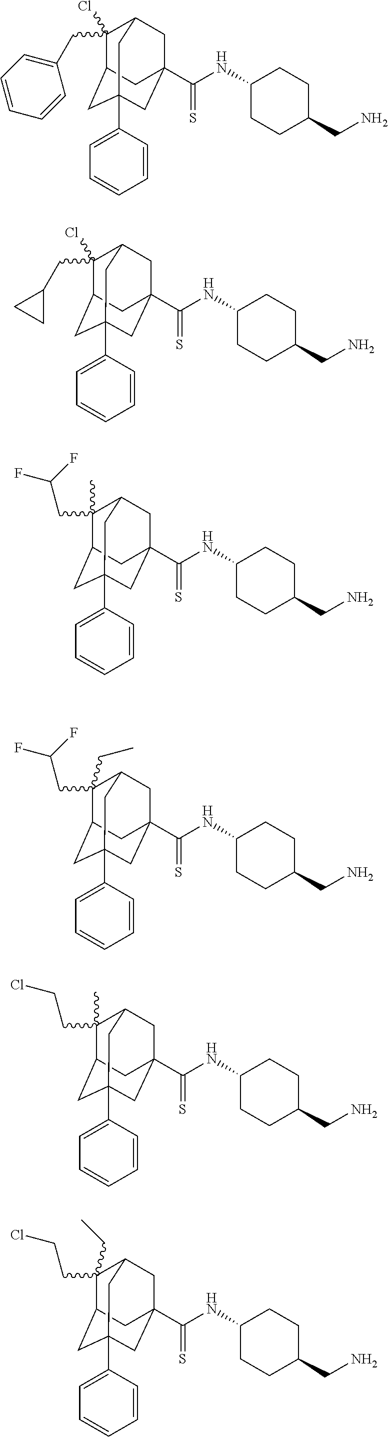

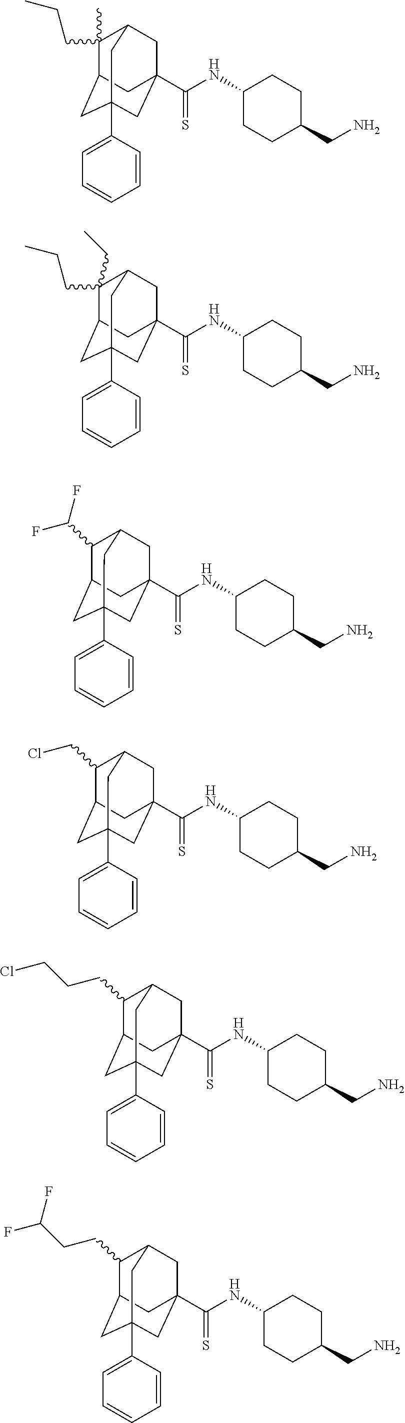

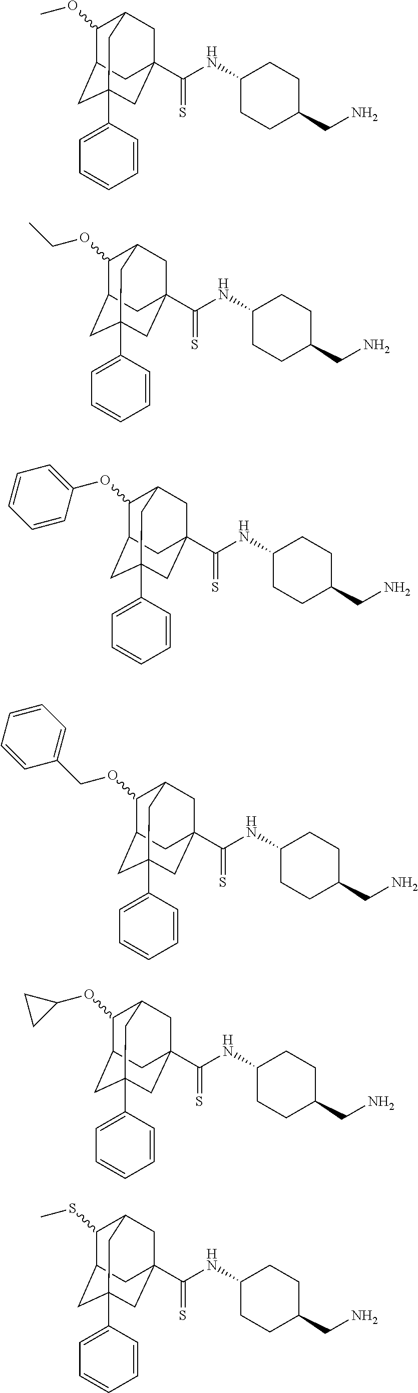

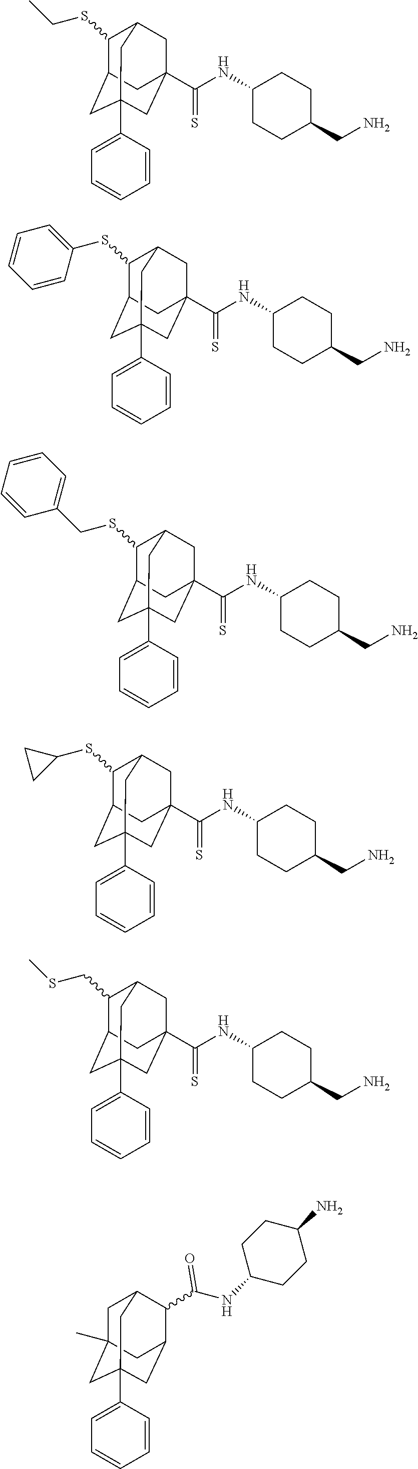

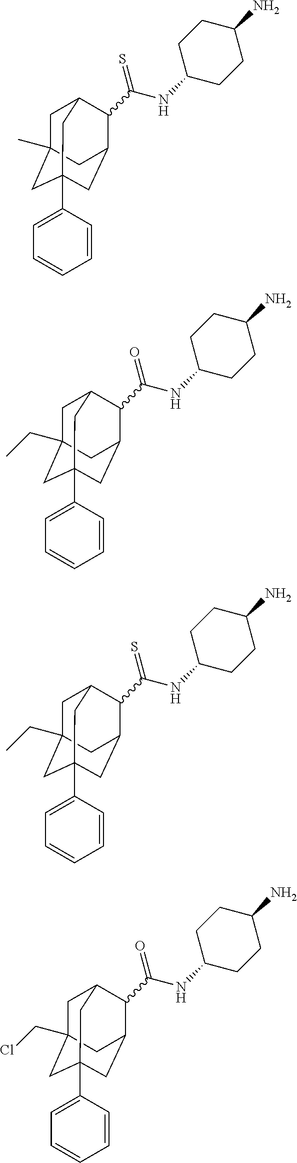 [SMILES: CC12CC3CC(c4ccccc4)(CC(C1)C3C(=S)N[C@H]1CC[C@H](N)CC1)C2 ; CCC12CC3CC(c4ccccc4)(CC(C1)C3C(=O)N[C@H]1CC[C@H](N)CC1)C2 ; CCC12CC3CC(c4ccccc4)(CC(C1)C3C(=S)N[C@H]1CC[C@H](N)CC1)C2 ; N[C@H]1CC[C@H](NC(=O)C2C3CC4(CCl)CC2CC(c2ccccc2)(C3)C4)CC1]

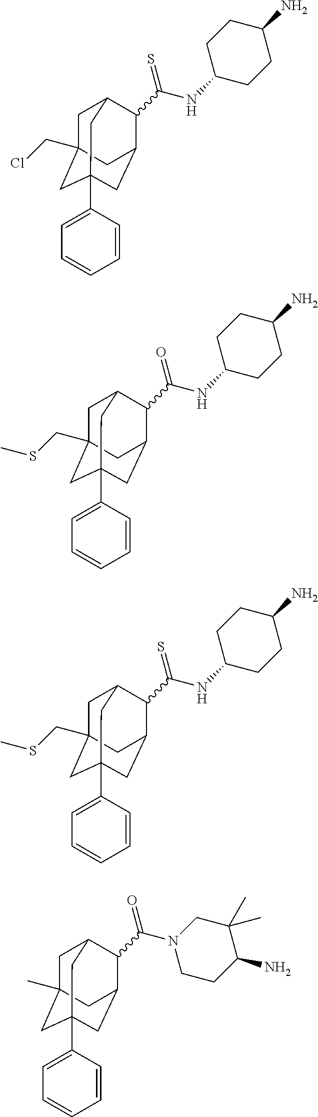 [SMILES: N[C@H]1CC[C@H](NC(=S)C2C3CC4(CCl)CC2CC(c2ccccc2)(C3)C4)CC1 ; CSCC12CC3CC(c4ccccc4)(CC(C1)C3C(=O)N[C@H]1CC[C@H](N)CC1)C2 ; CSCC12CC3CC(c4ccccc4)(CC(C1)C3C(=S)N[C@H]1CC[C@H](N)CC1)C2 ; CC12CC3CC(c4ccccc4)(CC(C1)C3C(=O)N1CC[C@H](N)C(C)(C)C1)C2]

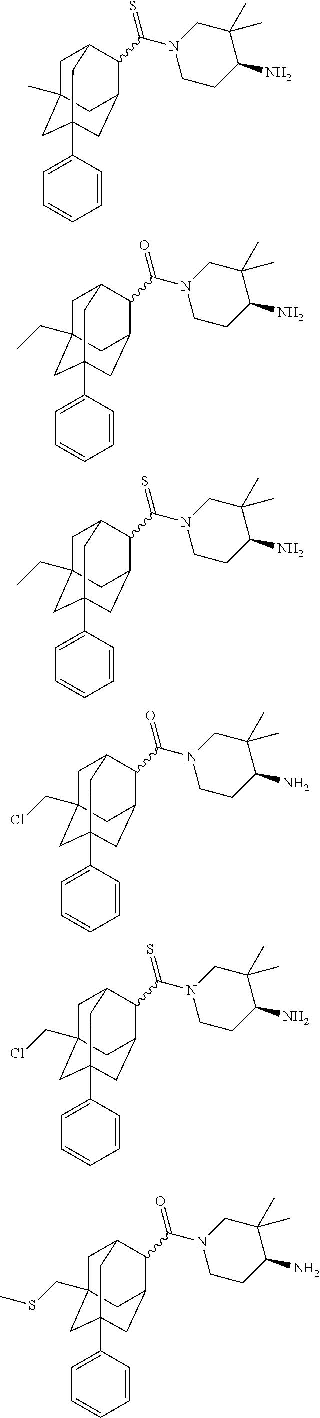

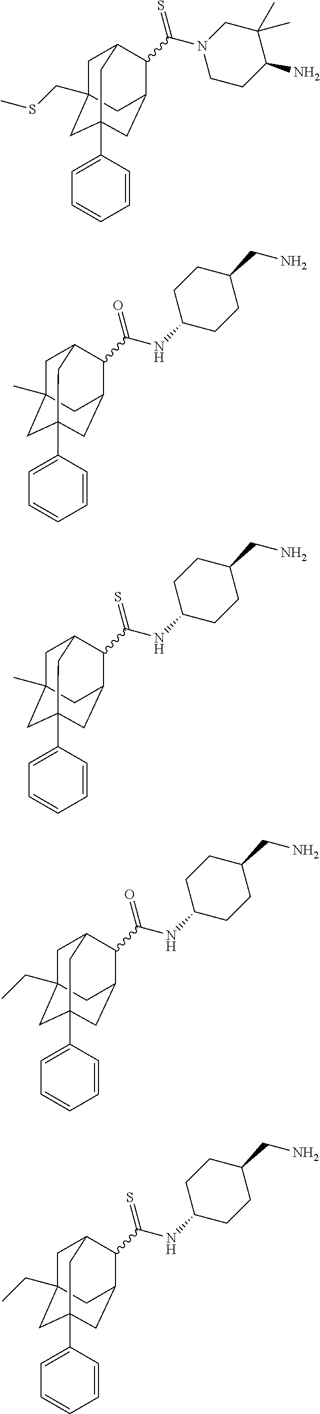 [SMILES: CSCC12CC3CC(c4ccccc4)(CC(C1)C3C(=S)N1CC[C@H](N)C(C)(C)C1)C2 ; CC12CC3CC(c4ccccc4)(CC(C1)C3C(=O)N[C@H]1CC[C@H](CN)CC1)C2 ; CC12CC3CC(c4ccccc4)(CC(C1)C3C(=S)N[C@H]1CC[C@H](CN)CC1)C2 ; CCC12CC3CC(c4ccccc4)(CC(C1)C3C(=O)N[C@H]1CC[C@H](CN)CC1)C2 ; CCC12CC3CC(c4ccccc4)(CC(C1)C3C(=S)N[C@H]1CC[C@H](CN)CC1)C2]

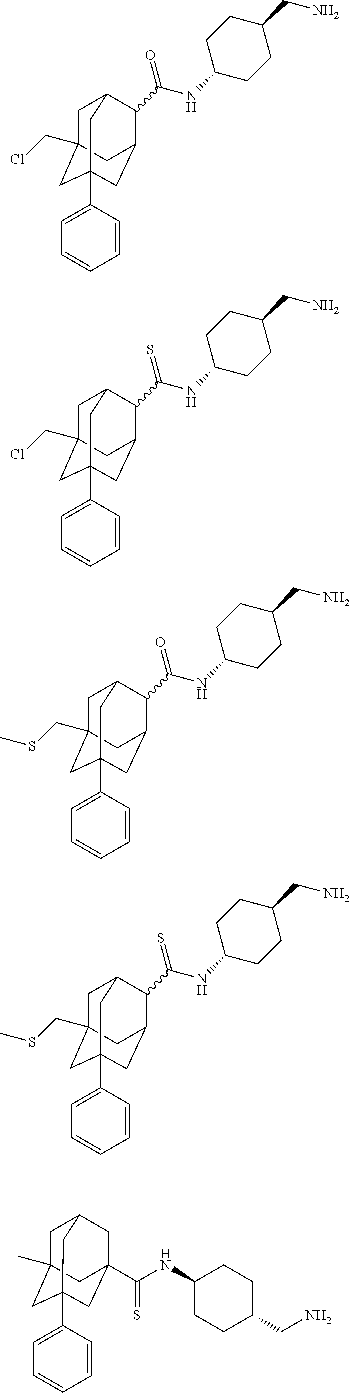 [SMILES: NC[C@H]1CC[C@H](NC(=O)C2C3CC4(CCl)CC2CC(c2ccccc2)(C3)C4)CC1 ; NC[C@H]1CC[C@H](NC(=S)C2C3CC4(CCl)CC2CC(c2ccccc2)(C3)C4)CC1 ; CSCC12CC3CC(c4ccccc4)(CC(C1)C3C(=O)N[C@H]1CC[C@H](CN)CC1)C2 ; CSCC12CC3CC(c4ccccc4)(CC(C1)C3C(=S)N[C@H]1CC[C@H](CN)CC1)C2 ; CC12CC3CC(C(=S)N[C@H]4CC[C@H](CN)CC4)(C1)CC(c1ccccc1)(C3)C2]

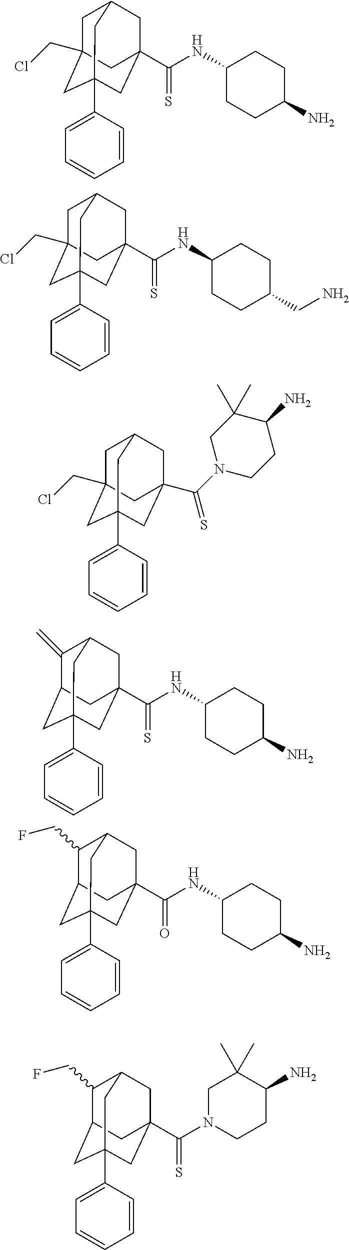

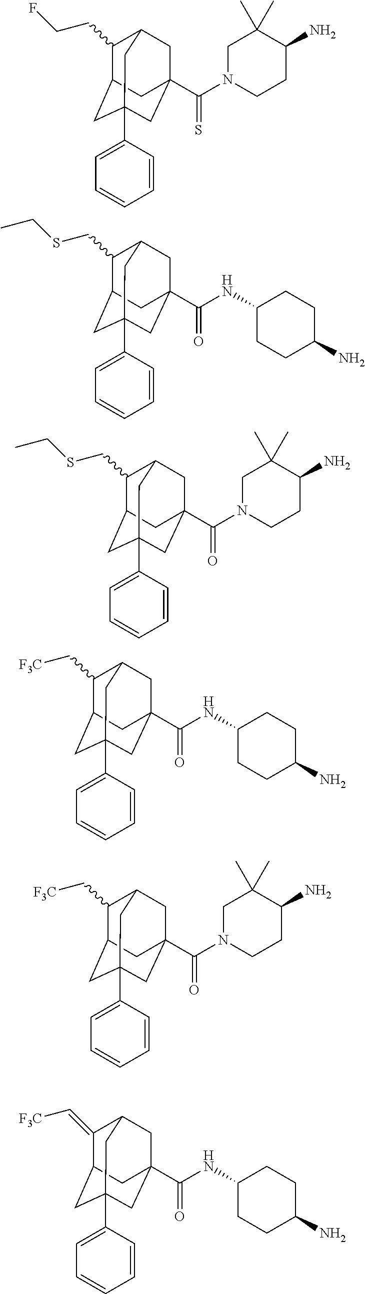

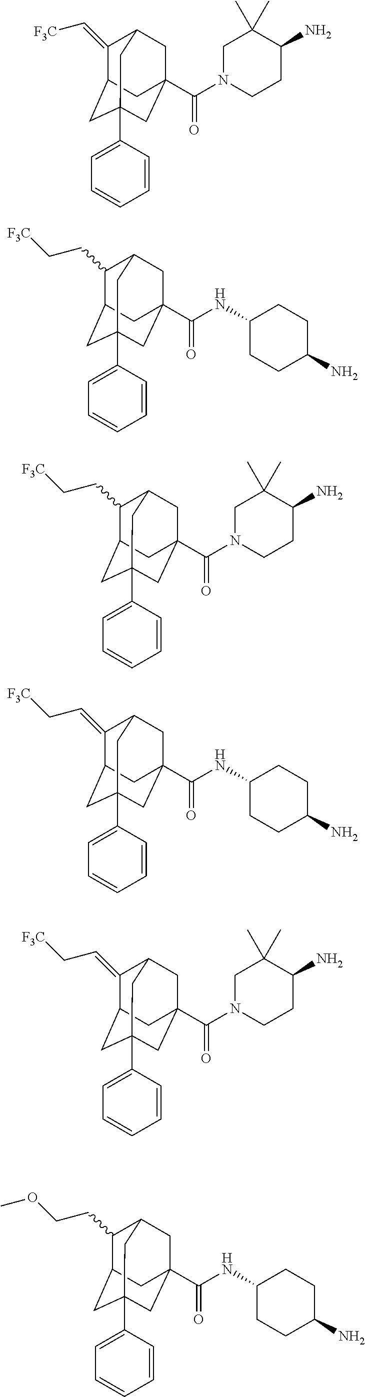 [SMILES: CC1(C)CN(C(=O)C23CC4CC(c5ccccc5)(CC(C2)C4=CC(F)(F)F)C3)CC[C@@H]1N ; N[C@H]1CC[C@H](NC(=O)C23CC4CC(c5ccccc5)(CC(C2)C4CCC(F)(F)F)C3)CC1 ; CC1(C)CN(C(=O)C23CC4CC(c5ccccc5)(CC(C2)C4CCC(F)(F)F)C3)CC[C@@H]1N ; N[C@H]1CC[C@H](NC(=O)C23CC4CC(c5ccccc5)(CC(C2)C4=CCC(F)(F)F)C3)CC1 ; CC1(C)CN(C(=O)C23CC4CC(c5ccccc5)(CC(C2)C4=CCC(F)(F)F)C3)CC[C@@H]1N ; COCCC1C2CC3(C(=O)N[C@H]4CC[C@H](N)CC4)CC1CC(c1ccccc1)(C2)C3]

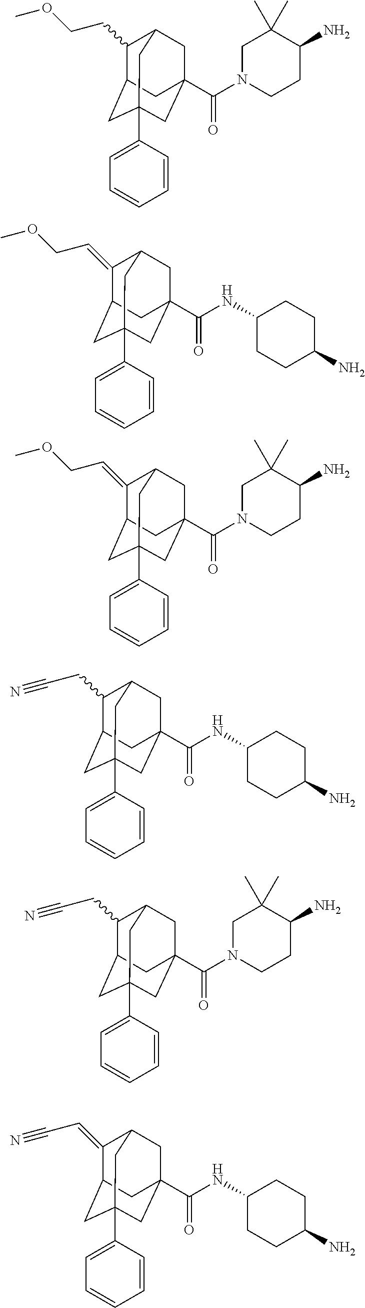

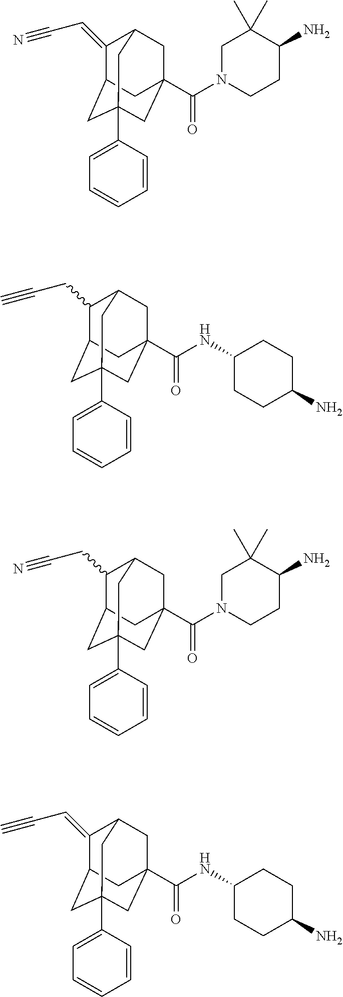 [SMILES: CC1(C)CN(C(=O)C23CC4CC(c5ccccc5)(CC(C2)C4=CC#N)C3)CC[C@@H]1N ; C#CCC1C2CC3(C(=O)N[C@H]4CC[C@H](N)CC4)CC1CC(c1ccccc1)(C2)C3 ; CC1(C)CN(C(=O)C23CC4CC(c5ccccc5)(CC(C2)C4CC#N)C3)CC[C@@H]1N ; C#CC=C1C2CC3(C(=O)N[C@H]4CC[C@H](N)CC4)CC1CC(c1ccccc1)(C2)C3]

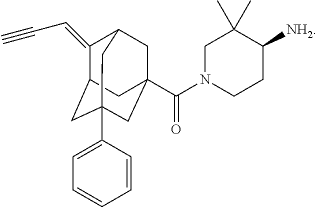 [SMILES: C#CC=C1C2CC3(C(=O)N4CC[C@H](N)C(C)(C)C4)CC1CC(c1ccccc1)(C2)C3]

In some embodiments, the invention provides for methods of treating infection by members of the Filoviridae family, which includes without limitation Ebolavirus, Marburgvirus, Cuevavirus, or any newly emerging filovirus genera. Five species of Ebolavirus have been identified: Zaire (EBOV), Bundibugyo (BDBV), Tai Forest (TAFV), Sudan (SUDV), and Reston (RESTV). Two species of Marburgvirus have been identified: (MARV) and Ravn (RAVV). One species of Cuervavirus has currently been identified: Lloviu virus (LLOV).

In some embodiments, the compounds of the invention can selectively inhibit Ebolavirus infection. Infection by Ebolavirus in humans leads to Ebola Hemorrhagic Fever (EHF), the clinical manifestations of which are severe and/or fatal. The incubation period varies between four and sixteen days. The initial symptoms are generally a severe frontal and temporal headache, generalized aches and pains, malaise, and by the second day the victim will often have a fever. Later symptoms include watery diarrhea, abdominal pain, nausea, vomiting, a dry sore throat, and anorexia. By day seven of the symptoms, the patient will often have a maculopapular (small slightly raised spots) rash. At the same time the person may develop thrombocytopenia and hemorrhagic manifestations, particularly in the gastrointestinal tract, and the lungs, but it can occur from any orifice, mucous membrane or skin site. Ebolavirus infections may cause lesions in almost every organ, although the liver and spleen are the most noticeably affected. Both are darkened and enlarged with signs of necrosis. The cause of death (>75% in most outbreaks) is normally shock, associated with fluid and blood loss into the tissues. The hemorrhagic and connective tissue complications of the disease are not well understood, but may be related to onset of disseminated intra-vascular coagulation. Infectious virus may linger in some tissues of some infected individuals for weeks and months after the intial infection.

In some embodiments, the compounds of the invention may inhibit infection by any virus, whether native or engineered, whose cell entry process is mediated by filovirus or hybrid filovirus glycoproteins.

### Exemplary Kits

The invention also includes kits. The kit has a container housing an inhibitor of the invention and optionally additional containers with other therapeutics such as antiviral agents or viral vaccines. The kit also includes instructions for administering the component(s) to a subject who has or is at risk of having an enveloped viral infection.

In some aspects of the invention, the kit can include a pharmaceutical preparation vial, a pharmaceutical preparation diluent vial, and inhibitor. The vial containing the diluent for the pharmaceutical preparation is optional. The diluent vial contains a diluent such as physiological saline for diluting what could be a concentrated solution or lyophilized powder of inhibitor. The instructions can include instructions for mixing a particular amount of the diluent with a particular amount of the concentrated pharmaceutical preparation, whereby a final formulation for injection or infusion is prepared. The instructions may include instructions for use in an oral formulation, inhaler, intravenous injection or any other device useful according to the invention. The instructions can include instructions for treating a patient with an effective amount of inhibitor. It also will be understood that the containers containing the preparations, whether the container is a bottle, a vial with a septum, an ampoule with a septum, an infusion bag, and the like, can contain indicia such as conventional markings which change color when the preparation has been autoclaved or otherwise sterilized.

### Protocol a for Pseudotype Inhibitory Testinq of Compounds.

Utilizing a VSV pseudotype system we previously screened a library collection of small molecule compounds [Cote, M.; Misasi, J.; Ren, T.; Bruchez, A.; Lee, K.; Filone, C. M.; Hensley, L.; Li, Q.; Ory, D.; Chandran, K.; Cunningham, J. Small molecule inhibitors reveal Niemann-Pick C1 is essential for Ebola virus infection, Nature (2011) 477: 344-348; Chandran, K.; Sullivan, N. J.; Felbor, U.; Whelan, S. P.; Cunningham, J. M. Endosomal proteolysis of the Ebola virus glycoprotein is necessary for infection, Science 2005 308:1643-1645] to discover adamantane carboxamides (PCT patent application, publication number PCT/US2017/013560, 13 Jan. 2017; WO 2017/127306, 27 Jul. 2017) that selectively inhibit viruses expressing filovirus glycoproteins and not viruses expressing glycoproteins from other viral families. The above papers and patent application are herein incorporated by reference in their entirety for all purposes. Compounds of the current invention were discovered through the use of similar pseudotyped viruses. Pseudotyped VSV viruses expressing the full-length VSV glycoprotein, as well as all pseudotyped VSV viruses expressing the other viral glycoproteins, were generated in cultured HEK-293T cells (ATCC CRL-3216). HEK cells were grown in 10 cm dishes in DMEM supplemented with 10% FBS, 1× Pen-Strep, sodium pyruvate, non-essential amino acids and L-glutamine. When cells reached approximately 80% confluency, they were transfected with a mixture of 15 μg of the pCAGGS plasmid encoding one of the desired glycoproteins, including native VSV or mucin-deleted EBOV [Genbank: AAB81004] or mucin-deleted BDBV [Genbank: AGL73453], or a full length EBOV [Genbank: AAB81004], SUDV [Genbank: YP_138523.1] or MARV [Genbank: AAC40460] glycoprotein construct, and 45 μl of PEI (polyethylenimine) transfection reagent. The cells were incubated with the solution for 5 hours at 37° C. at 5% CO₂. The cells were then washed and the mixture replaced with supplemented DMEM and incubated at 37° C. at 5% CO₂ for approximately 16-18 hours. Subsequently cells were infected with approximately 50 μl of VSV parent pseudotype virus lacking VSV glycoprotein and containing the gene for luciferase. The cells were infected for 1 hour, then washed 1× with PBS and incubated in supplemented media. 24 hours post-infection, supernatant was collected, aliquoted and stored at −80° C. For VSV-Luciferase pseudotypes, one aliquot was thawed and tested in a serial dilution for luminescence activity in Vero cells as described in the Luciferase assay protocol (below). Each of the viral supernatants generated was diluted (from 1:100 to 1:2000) to give similar luminescence signal/background values of ≥200 and stored at −80° C. as aliquots for later use. Vero cells (ATCC: CCL-81) were grown in clear 384 well plates (3000 cells/well) in DMEM media with 10% FBS, 1× Pen-Strep, sodium pyruvate, non-essential amino acids and L-glutamine. After incubating overnight at 37° C. and 5% CO₂, cells were treated with compounds at desired concentrations and pseudotyped virus in assay media. Assay media consisted of 50% Opti-MEM, 50% DMEM, with 1% FBS, Pen-Strep, sodium pyruvate, non-essential amino acids and L-glutamine. Final DMSO concentration in the compound testing wells was kept≤1% and control wells were treated with assay media and 1% DMSO. Cells were incubated for 24 hours at 37° C. and 5% CO₂. The compound-virus mixture was aspirated off the cells 24 hours post-infection and washed 1× with PBS. Cells were lysed using 20 μl of lysis buffer from a Luciferase kit diluted according to manufacturer's (Thermo Scientific) instructions. After incubating for approximately 20 minutes, 5 μl of cell lysate was transferred to an opaque white plate and mixed with 12.5 μl of Coelenterazine diluted in buffer. This mixture was incubated at room temperature for 10 minutes on a plate shaker, and then the luminescence was read using a plate reader (Beckman Coulter DTX 880 multimode detector with an emission of 535 nm) Luminescence signals were obtained for compound containing and control wells to determine % activity (inhibition of luciferase signal) for each compound. The 50% effective (EC₅₀, virus-inhibitory) concentrations were calculated using non-linear regression analysis on GraphPad PRISM software (version 9.02).

We previously determined (PCT patent application, publication number PCT/US2017/013560, 13 Jan. 2017; WO 2017/127306, 27 Jul. 2017) that adamantyl carboxamides do not inhibit native VSV (expressing the native glycoprotein) and believe that adamantyl thioamides would follow the same trend due to structural similarities between these two series of compounds. The above patent application is herein incorporated by reference in its entirety for all purposes. Compounds were tested in the pseduotyped assays in dose-response experiments to determine EC₅₀ values (concentration at half-maximal inhibition) and those exhibiting an EC₅₀≥10-fold below the concentration of half-maximal cell death (CC₅₀), as determined in parallel cytotoxicity assays, were thereby identified as filovirus cell entry inhibitors. Compounds exhibiting activity against one or more pseudotyped filoviruses without comparable cytotoxicity (or VSV activity), indicates they are of potential therapeutic interest to treat filovirus infection. For the cytotoxicity asays compounds were serially diluted and added to Vero cells (6000 cells/well) with final DMSO concentration maintained at 1% in growth media consisting of DMEM with 2% FBS. The plates were incubated at 37° C. for 5 days, and then dead cells were removed by washing with Phosphate buffered saline (PBS). Cells were stained with neutral red vital dye for 1 hour and then de-stained with a solution of 50% ethanol/1% acetic acid solution. Absorbance was read at 540 nm and 690 nm on a Spectramax Plus 384 spectrophotometer. Data were analyzed as (540 nm-690 nm) and then compared to untreated controls to obtain % cell viability. CC₅₀s were calculated using non-linear regression analysis on GraphPad PRISM software (version 9.02).

### Microsomal Assays

In addition to the ability of compounds to inhibit live filoviruses in vitro, compounds must also have certain drug-like properties for them to be used to inhibit filoviruses and provide methods of treatment for filovirus infection in mammals in vivo. Such compounds may exhibit drug-like properties including but not limited to chemical stability against metabolic degradation by liver microsomal CYP p450 enzymes, cell permeability and oral bioavailability (if the drug is to delivered orally) and lack of inhibition of the hERG ion channel, which is associated with cardiac safety [Kerns, E. H. Li, D. Drug-like Properties: Concepts, Structure Design and Methods from ADME to Toxicity Optimization, (2008) Academic Press, Burlington MA]. The above publication is herein incorporated by reference in its entirety for all purposes. To characterize drug-like properties of the chemical series example compounds were evaluated for metabolic stability in human, mouse, guinea pig, monkey, rat, and dog liver microsome assays (Table 11). Compounds exhibiting>60% remaining of parent indicate attractive chemical stability. The demonstration of good microsomal stability in human and nonhuman species facilitates the ability to test and optimize compounds in preclinical animal studies. To reduce or prevent serious/life-threatening conditions caused by exposure to lethal or permanently disabling toxic agents where human efficacy trials are not feasible or ethical (such as filovirus infection) the FDA has provided an approach to test and approve drugs using the Animal Efficacy Rule; whereby the FDA can rely on evidence from animal studies to provide substantial evidence of product effectiveness. In the absence of an epidemic filovirus outbreak in humans with a sufficiently large patient population efficacy data for new methods of treatment for filovirus infection may only be obtained from relevant animal models (e.g., mouse and monkey efficacy studies). Thus, the translation of drug like-properties from one species to another significantly facilitates the testing and development of filovirus inhibitor compounds.

A reaction premixture was set up, containing 1 uM compound of interest, 1 mg/mL liver microsomes of desired species, 2.1 mM MgCl₂ and 0.1 M sodium phosphate buffer, pH 7.4. This premixture was incubated at 37° C. for 15 minutes with gentle agitation to allow the compound to be completely dissolved in the mixture. Then freshly made NADPH solution in 0.1 M sodium phosphate buffer was added at a concentration of 2 mM to start the reaction. A ‘Time 0’ sample (30 uL) was taken out immediately after addition of NADPH and added to 140 uL cold acetonitrile containing 1 uM of pre-decided internal standard. The rest of the reaction mixture was incubated at 37° C. for the remaining time period. Test compounds were left in the reaction mixture for 60 minutes before ‘Time 60’ sample was added to acetonitrile with internal standard. The control compound (Verapamil for human, monkey and dog LM, Lidocaine for Guinea pig LM, and diphenhydramine for rat and mouse LM) was incubated in reaction mixture for 15 minutes before ‘Time 15’ samples were collected and added to cold acetonitrile with internal standard. The samples were then spun in a centrifuge for 10 minutes at 4000 rpm, supernatant was collected and mixed with equal parts distilled water. These were then analyzed on a Varian 500-MS.

TABLE 6
Pseudotyped Virus Activity. Example compounds and
their observed inhibitory activities are shown as
EC₅₀ values for pseudotyped EBOV, BDBV, SUDV, and MARV and
CC₅₀ for cytotoxicity; nd: not determined.

| | EC₅₀ (uM) Pseudotype Assays | | | | Cytotox (uM) |
|---|---|---|---|---|---|
| Ex. | EBOV | BDBV | SUDV | MARV | CC₅₀ |
| A1 | 0.061 | 0.11 | 0.043 | >1 | 3.8 |
| B1 | 0.74 | 0.40 | 0.26 | nd | nd |
| C1 | 0.06 | 0.085 | 0.035 | nd | nd |
| A2 | 0.041 | 0.065 | 0.056 | >1 | 5.1 |
| B2 | 0.09 | 0.16 | 0.26 | nd | 3.72 |
| C2 | 0.06 | 0.085 | 0.07 | nd | nd |
| A3 | 0.067 | 0.29 | 0.08 | >1 | 1.77 |
| B3 | 0.66 | 0.31 | 0.36 | >1 | nd |
| C3 | 0.17 | 0.24 | 0.1 | nd | nd |
| A4 | 0.085 | 0.06 | 0.04 | nd | 4.09 |
| A5 | 0.12 | 0.19 | 0.125 | nd | nd |
| A6 | 0.064 | 0.085 | 0.08 | nd | 4.72 |
| A7 | 0.075 | 0.09 | 0.095 | nd | 2.86 |
| D1 | 0.21 | 0.30 | 0.29 | nd | nd |
| D2 | 0.051 | nd | 0.072 | nd | 1.78 |
| E1 | 0.034 | 0.12 | 0.56 | >1 | 13.8 |
| E2 | 0.057 | 0.35 | 0.165 | >1 | 36.9 |
| E3 | 0.061 | 0.19 | 0.34 | nd | nd |
| E6 | 0.05 | 0.6 | 0.75 | >1 | 34.6 |
| E7 | 0.056 | 1.21 | >1 | >1 | 4.75 |
| E8 | 0.145 | >1 | >1 | >1 | nd |
| F1 | 0.027 | 0.065 | 0.21 | >3 | 13.34 |
| F2 | 0.011 | 0.03 | 0.27 | 3.1 | 12.63 |
| F3 | 0.005 | 0.08 | 0.39 | 1.6 | 4.65 |
| F4 | 0.016 | 0.3 | 0.86 | 1.38 | 5.8 |
| F5 | 0.046 | nd | 0.1 | nd | 12.27 |
| G1 | 0.01 | 0.11 | 0.56 | >3 | 14.88 |
| G2 | 0.005 | 0.1 | 0.18 | 1.85 | 3.79 |
| G3 | 0.008 | 0.28 | 0.87 | 1.81 | 17.9 |
| G4 | >1 | >1 | >1 | nd | nd |
| G5 | 0.069 | nd | 0.12 | nd | 6.85 |
| G6 | 0.043 | nd | >1 | nd | 7.59 |
| G7 | 0.016 | nd | 0.037 | nd | 6.24 |
| G8 | 0.2 | nd | >1 | nd | 24.01 |
| G9 | 0.021 | nd | 0.11 | nd | 2.58 |
| G10 | >1 | nd | >1 | nd | nd |
| G11 | 0.016 | nd | 0.03 | nd | 9.86 |
| G12 | 0.07 | nd | 0.21 | nd | 5.62 |
| G13 | 0.051 | nd | nd | nd | 32.49 |
| G14 | 0.104 | nd | nd | nd | 3.99 |
| G15 | 0.122 | nd | nd | nd | 8.29 |
| G16 | 0.006 | nd | nd | nd | 9.44 |
| G17 | 0.092 | nd | nd | nd | nd |
| G18 | 0.002 | nd | 0.1 | nd | 7.69 |
| G19 | 0.028 | nd | 0.096 | nd | 7.37 |
| H1 | >1 | nd | >1 | nd | nd |
| H2 | >1 | nd | >1 | nd | nd |
| H3 | 0.063 | nd | 0.17 | nd | 14.69 |
| H4 | 0.014 | nd | 0.16 | nd | 25.14 |
| H5 | 0.01 | nd | 0.27 | nd | 7.265 |
| H6 | 0.02 | nd | 0.18 | nd | 22.19 |

CC₅₀ in Vero cells (120 h)

As shown in Table 6, compounds of the invention exhibited inhibition of pseudotyped viruses expressing Ebolavirus glycoproteins well below that of cytotoxicity. Thioamides prepared from enantiomerically pure (1 S,3R,5R,7S)-3-methyl-5-phenyl adamantane-1-carboxylic acid (examples A1 to A3) were surprisingly and significantly more potent than the opposite enantiomer prepared from (1R,3S,5S,7R)-3-methyl-5-phenyl adamantane-1-carboxylic acid (examples B1 to B33) with eudistic ratios ranging from 2 to 12 for Ebola virus (EBOV) and 5 to 7 for Sudan virus (SUDB). Unexpectedly, a number of tested compounds of the invention did not exhibit inhibition of pseudotyped virus expressing Marburgvirus glycoprotein. These unexpected results provide strong support for the development of adamantane carboxamides and thioamides for the treatment of Ebolavirus infection.

### Protocol B—Native Ebola and Sudan Immuno-Fluorescence Staining Assays

A high-throughput, high-content imaging based phenotypic screening assay was used to identify activity against EBOV (strain kikwit) and SUDV (strain Gulu). HeLa cells were maintained and propagated according to manufacturer's (ATCC) recommendations. Cells were plated (seeding density 2000) in 384-well imaging plates and incubated for 20-24 hours prior to treatment with the compound. For EC50 and CC50 determination, the HP-D300 digital dispenser (Hewlett Packard) or the Janus robotic liquid handling system (Perkin Elmer) was used to generate 8-point dose response with a 3-fold step dilution. Each dose was dispensed in triplicate, with final DMSO concentration at 0.5%. At least one positive control compound was selected for use as an internal reference inhibitor on each plate in the dose response assays.

Two hours after treatment, assay plates were transferred to biosafety level (BSL)-4. Cells in assay plates were infected at a multiplicity of infection (MOI), selected based on optimization data, to achieve 60-90% infection rate in control wells at the assay endpoint. Following virus inoculation, assay plates were incubated at 37° C. with 5% CO2 for 20-48 hours (depending on the virus used). Cells were then fixed in 10% buffered formalin for at least 48 hours before immunostaining.

Inactivated plates were transferred to the BSL-2 lab for immunostaining. Assay wells were incubated with permeabilization/blocking buffer containing 3% BSA/0.1% Trition/PBS for 1 hour. Assay wells were then stained for 1 hour with a primary antibody against the virus tested, diluted 1,000-fold in blocking buffer. Following incubation, the primary antibody was removed and the cells washed 3 times with 1×PBS. Cells were subsequently incubated for 1 hour with DyLight-488-conjugated goat anti-mouse IgG (Thermo Fisher) diluted 1,000-fold in blocking buffer. Cells were stained with Hoechst3332 (Thermo Fisher) for nuclei detection and CellMask Deep Red (Thermo Fisher) for optimal detection of cytoplasm for at least 30 min before image acquisition.

Images were acquired on the Opera confocal imaging instrument (Perkin Elmer) using 10× Air objective and five fields typically acquired per well. Signal from virus staining was detected by CCD cameras at 488 nm emission wavelength, nuclei staining—at 400 nm and cytoplasm staining—at 640 nm.

Image analysis was performed simultaneously with image acquisition using PE Acapella algorithms. The percentage (%) of infected cells was calculated by the Acapella algorithm for each well directly. Dose response curve analysis (to determine EC50 values) was performed using GeneData Screener software applying Levenberg-Marquardt algorithm (LMA) for curve-fitting strategy.

TABLE 7
Inhibition of Native Ebola (Kikwit) and Sudan (Gulu) Viruses. Example
compounds and their observed inhibitory activities and selectivity
indices (SI) in an Immuno-fluorescence staining assay.

| | | EBOV-Kikwit | | SUDV-Gulu | |
|---|---|---|---|---|---|
| | Assay | EC₅₀ | SI₅₀ | EC₅₀ | SI₅₀ |
| Example | Format | (uM) | (CC₅₀/EC₅₀) | (uM) | (CC₅₀/EC₅₀) |
| G1 | Immunostaining | 0.12 | >212.23 | 0.042 | >447.44 |
| F2 | Immunostaining | 0.11 | 90.33 | 0.039 | 291.26 |

Some Ebola entry inhibitor compounds identified from pseudotype virus assays were tested for efficacy against wild-type Ebola and Sudan viruses in the immuno-fluorescence staining assay (Table 7). The SI₅₀ selectivity index (=CC₅₀/EC₅₀) is typically used to determine whether a compound is exhibiting true antiviral inhibitory and SI₅₀ values>10 are accepted as confirmation of inhibitory activity against the virus, rather than artifactual activity reflecting cellular cytotoxicity. Both compounds exhibited potent EC₅₀ values of 110-120 nM for EBOV and 39-42 nM for SUDV. SI₅₀ values were >90.33 for EBOV and >291.26 for SUDV clearly indicating compound efficacy was due to antiviral activity and not cytotoxic effects. The results shown in Tables 6 and 7 confirm the activity of the compounds against Ebolaviruses including the replicative EBOV and SUDV and also strongly validates the approach of identifying and prioritizing bona fide Ebolavirus inhibitors through the utilization of pseudotyped virus assays.

### Protocol C—Native Ebola Plaque and Viral Yield Reduction Assays.

Biosafety Safety Level 2 (BSL2) pseudotyped viruses expressing filovirus GPs were used (above) as surrogates to facilitiate the identification of inhibitors of wild-type Biosafety safety level 4 (BSL4) filoviruses, which may only be studied in highly specialized containment facilities. To confirm activity against native BSL4 Ebola virus example compounds were tested against EBOV and SUDV in plaque forming assay format under stringent BSL4 testing requirements. In the plaque assay format confluent or near confluent (Vero) cell culture monolayers in 12-well disposable cell culture plates are prepared. Cells are maintained in MEM or DMEM supplemented with 10% FBS. For antiviral assays the same medium is used but with FBS reduced to 2% or less and supplemented with 1% penicillin/streptomycin. The test compound is prepared at four log₁₀ final concentrations in 2×MEM or 2×DMEM. The virus only and cytotoxicity (compound only) controls are run in parallel with each tested compound. Further, a known active drug (favipiravir) is tested as a positive control drug with each test run. Test compounds and positive controls are tested in biological triplicates. The assay is initiated by first removing growth media from the 12-well plates of cells, and infecting cells with 0.01 MOI of virus or about 50 to 100 plaque forming units (pfu). Cells are incubated for 60 min: 100 μl inoculum/well, at 37° C., 5% CO₂ with constant gentle rocking. Virus inoculum is removed, cells washed and overlaid with either 1% agarose or 1% methylcellulose diluted 1:1 with 2×MEM and supplemented with 2% FBS and 1% penicillin/streptomycin and supplemented with the corresponding drug concentration. Cells are incubated at 37° C. with 5% CO₂ for 10 days. The overlay is removed and plates stained with 0.05% crystal violet in 10% buffered formalin for approximately twenty minutes at room temperature. The plates are washed, dried and the number of plaques counted. The number of plaques in each set of compound dilution is converted to a percentage relative to the untreated virus control. The 50% effective (EC₅₀ virus-inhibitory) concentration is calculated by linear regression analysis. The cytotoxicity assay (In vitro Toxicology Assay Kit, Neutral red based; Sigma) is being performed in parallel in 96-well plates following the manufacturer's instructions. Briefly, growth medium is removed from confluent cell monolayers and replaced with fresh medium (total of 100 μL) containing the test compound with the concentrations as indicated for the primary assay. Control wells contain medium with the positive control or medium devoid of compound. A total of up to five replicates are performed for each condition. Plates are incubated for 3, 5, or 10 days at 37° C. with 5% CO₂. The plates are stained with 0.033% neutral red for approximately two hours at 37° C. in a 5% CO₂ incubator. The neutral red medium is removed by complete aspiration, and the cells rinsed 1× with phosphate buffered solution (PBS) to remove residual dye. The PBS is completely removed and the incorporated neutral red eluted with 1% acetic acid/50% ethanol for at least 30 minutes. Neutral red dye penetrates into living cells: the more intense the red color, the larger the number of viable cells present in the wells. The dye content in each well is quantified using a 96-well spectrophotometer at 540 nm wavelength and 690 nm wavelength (background reading). The 50% cytotoxic (CC₅₀, cell-inhibitory) concentrations are then calculated by linear regression analysis. The quotient of CC₅₀ divided by EC50 gives the selectivity index (SI₅₀) value.

TABLE 8
Inhibition of Native Ebola (Mayinga) Virus. Example compounds
and their observed inhibitory activities and selectivity
indices (SI) in a viral yield reduction (VYR) assay.

| | | WT EBOV-Mayinga | | | |
|---|---|---|---|---|---|
| | Assay | EC₅₀ | EC₉₀ | SI₅₀ | SI₉₀ |
| Compd. | format | (uM) | (uM) | (CC₅₀/EC₅₀) | (CC₅₀/EC₉₀) |
| G1 | VYR | ~0.008 | ~0.009 | >~3229 | >~2659 |
| F2 | VYR | 0.014 | 0.048 | 706.84 | 209.99 |
| G2 | VYR | 0.0004 | 0.075 | nd | nd |
| F3 | VYR | ~0.008 | ~0.01 | nd | nd |

TABLE 9
Inhibition of replicative Ebola (Kikwit) and Sudan (Gulu)
Viruses. Example compound A2 and its observed inhibitory activities
(EC₉₀) and selectivity indices (SI₉₀) in replicative EBOV
and SUDV viral yield reduction (VYR) assays.

| | EBOV-Kikwit VYR Assay | | SUDV-Gulu VYR Assay | |
|---|---|---|---|---|
| Example | EC₉₀ (uM) | SI₉₀ (uM) | EC₉₀ (uM) | SI₉₀ (uM) |
| A2 | 1.85 | 2.32 | 0.13 | 33.08 |

TABLE 10
Inhibition of Native Ebola (Zaire) and Sudan (Gulu) Viruses.
Example compounds and their observed inhibitory activities
and selectivity indices (SI) in a plaque assay.

| | | EBOV-Zaire | | SUDV-Gulu | |
|---|---|---|---|---|---|
| | Assay | EC₅₀ | SI₅₀ | EC₅₀ | SI₅₀ |
| Example | Format | (uM) | (CC₅₀/EC₅₀) | (uM) | (CC₅₀/EC₅₀) |
| D2 | Plaque | 2.6 | 3.3 | 0.11 | 78 |
| G7 | Plaque | 0.81 | 11 | 0.11 | 79 |
| G9 | Plaque | 0.98 | 9 | 0.73 | 12 |
| G11 | Plaque | 0.96 | 39 | 0.93 | 41 |

A further set of Ebola entry inhibitors identified from pseudotype virus cell assays were tested for efficacy against wild-type Ebola and Sudan species either by plaque or virus yield reduction (VYR) assays (Tables 8-10), according to the protocols discussed above.

TABLE 11
Multi-species Microsomal Stability. Percent parent compound
remaining at 60 minutes in liver microsomes

| Ex. | Human | Mouse | Monkey | Guinea Pig | Dog | Rat |
|---|---|---|---|---|---|---|
| A1 | 72 | nd | 19 | 32 | nd | nd |
| A2 | 100 | 3 | 85.5 | 100 | 95 | 86 |
| D2 | 100 | nd | 100 | 100 | nd | nd |
| E1 | 89 | 69 | 72 | 100 | nd | nd |
| E3 | 97.5 | nd | 91 | 96 | nd | nd |
| F1 | 91 | 83 | 92 | 79 | 82 | 99 |
| F2 | 85 | 48 | 100 | 68 | 80 | 86.5 |
| F3 | 91 | 26 | 100 | 48 | nd | nd |
| G1 | 100 | 17 | 70 | 70 | 56 | 100 |
| G2 | 96 | 17 | 92 | 85 | nd | nd |
| G5 | 100 | nd | 6 | 22 | nd | nd |
| G6 | 100 | nd | 100 | 100 | nd | nd |
| G7 | 100 | nd | 28 | 100 | nd | nd |
| G16 | 68 | 2 | nd | 59.5 | nd | nd |
| H4 | 81 | 39 | nd | 80.5 | nd | nd |
| H5 | 85 | 35 | nd | 80.5 | nd | nd |

In summary, example compounds of the invention exhibit potencies of low nanomolar EC₅₀ activity against pseudotyped viruses expressing a range of Ebolavirus glycoproteins and low nanomolar to sub uM EC₅₀ activities against native Ebola and Sudan viruses with selectivity indices that confirm them as bona fide Ebolavirus inhibitors. In addition, initial drug-like property characterization of example compounds indicates attractive microsome stability in human, mouse, monkey, rat, dog, and guinea pig (potential efficacy and toxicology animal models). These data indicate that the compounds of the invention may provide attractive therapeutics and method of treatment for Ebolavirus infection.

All publications and patents mentioned herein are hereby incorporated by reference in their entirety as if each individual publication or patent was specifically and individually indicated to be incorporated by reference. In case of conflict, the present application, including any definitions herein, will control.

What is claimed is:

1. A method of treating infections associated with viruses of the Ebolavirus enveloped virus, or any virus expressing filovirus glycoproteins to mediate cell entry comprising administration of a therapeutically effective amount of a compound of Structural Formula I 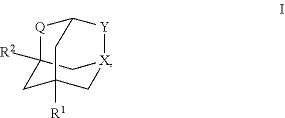
or a pharmaceutically acceptable salt, and a pharmaceutically acceptable carrier, diluent, or vehicle thereof, wherein:
X is 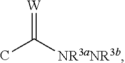
Y is CH₂, and Q is CH₂ or CR²³R²⁴; or
X is CH, Y is 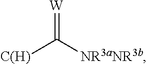
and Q is CH₂;
W is selected from the group consisting of O and S;
and when X is 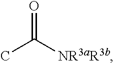
Y is CH₂, and Q is CH₂, then
R¹ is selected from (C₆ to C₁₀) aryl and (C₂ to C₉) heteroaryl, wherein
each of the said (C₆ to C₁₀) aryl and (C₂ to C₉) heteroaryl is optionally substituted with at least one R⁸ group;
R² is selected from the group consisting of 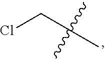 [SMILES: *CCl]
Br, and 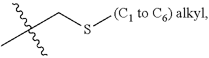
and
NR^{3a}R^{3b} is selected from the group consisting of 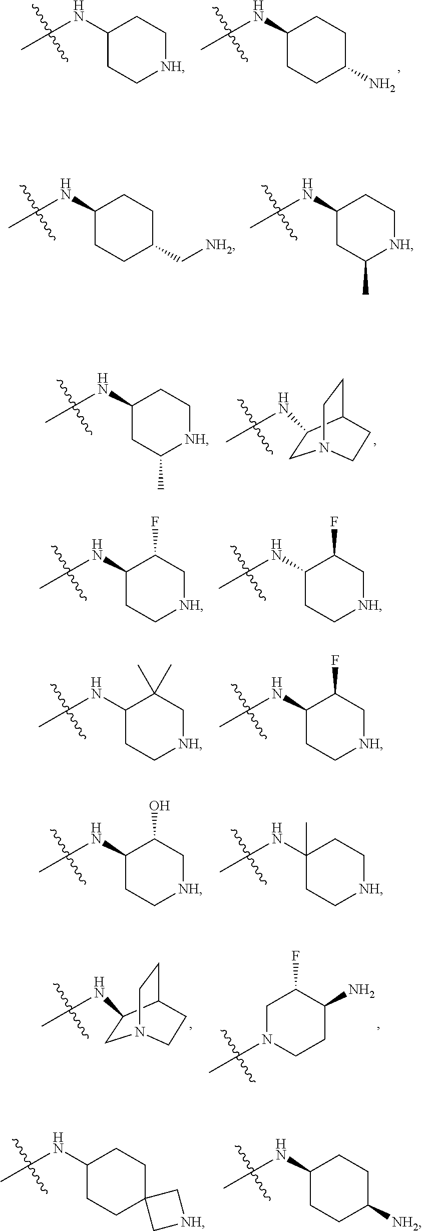 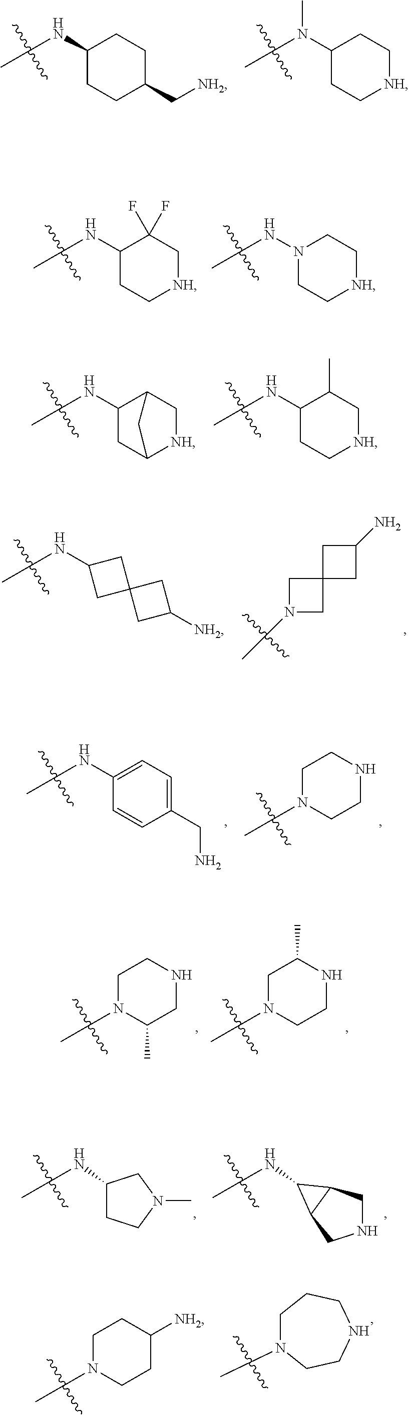 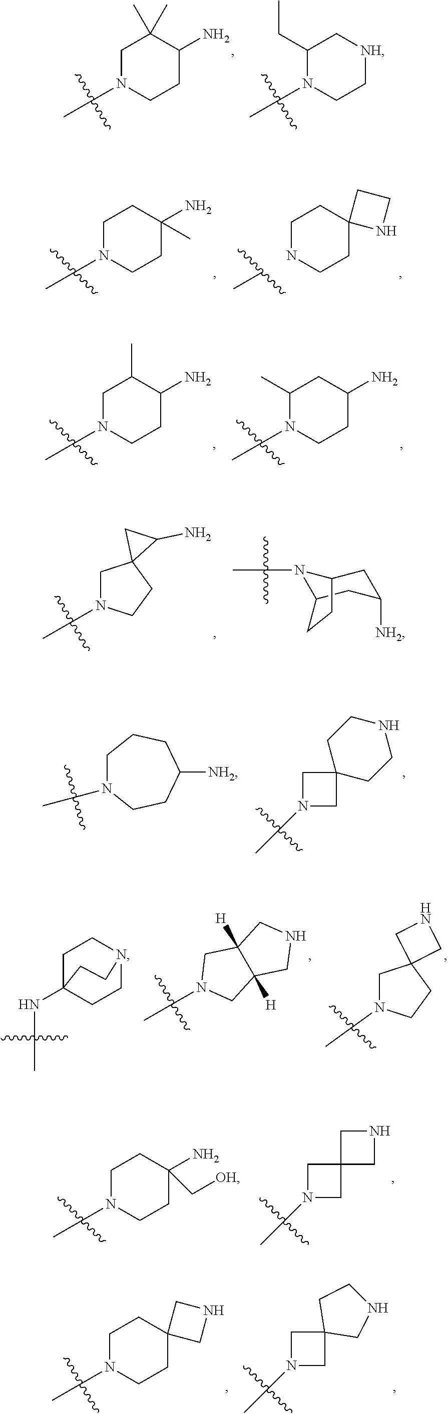 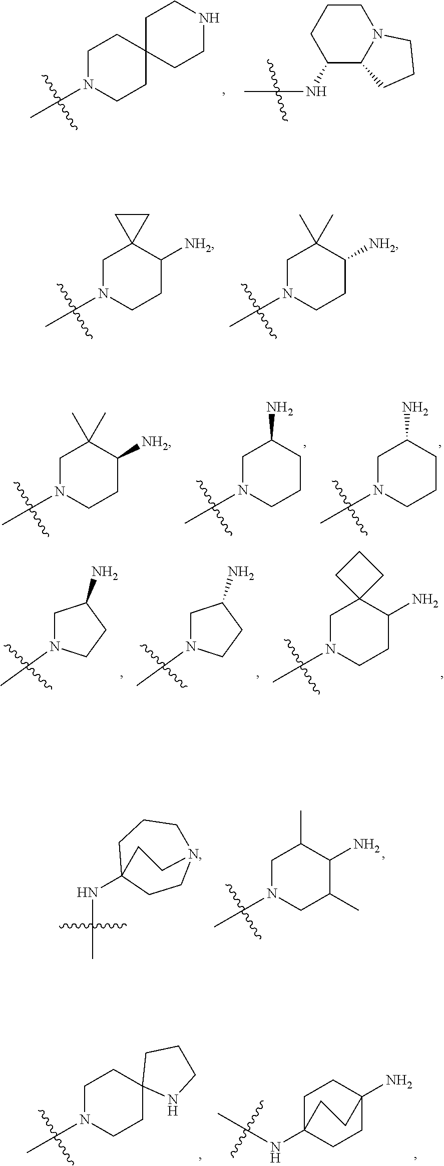 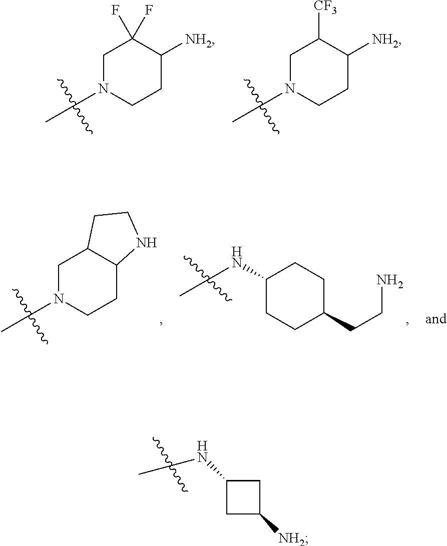 [SMILES: *N1CCC(N)C(F)(F)C1 ; *N1CCC(N)C(C(F)(F)F)C1 ; *N1CCC2NCCC2C1 ; *N[C@H]1CC[C@H](CCN)CC1 ; *N[C@H]1C[C@H](N)C1]
and when X is CH, Y is 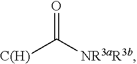
and Q is CH₂, then
R¹ is selected from (C₆ to C₁₀) aryl and (C₂ to C₉) heteroaryl, wherein
each of the said (C₆ to C₁₀) aryl and (C₂ to C₉) heteroaryl is optionally substituted with at least one R⁸ group;
R² is selected from the group consisting of halogen, methyl, ethyl, propyl, chloromethyl, chloroethyl, methylthiomethyl, and thiomethyl;
NR^{3a}R^{3b} is selected from the group consisting of 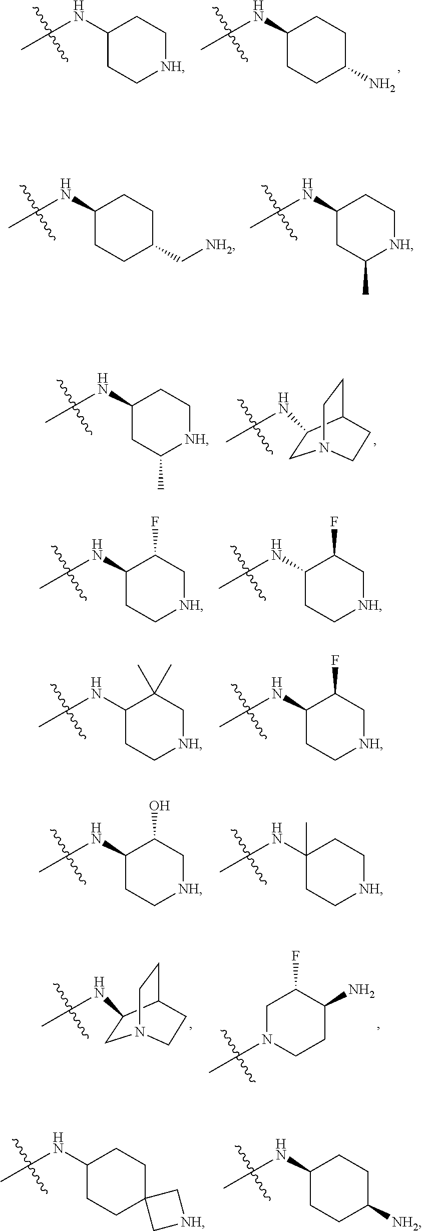 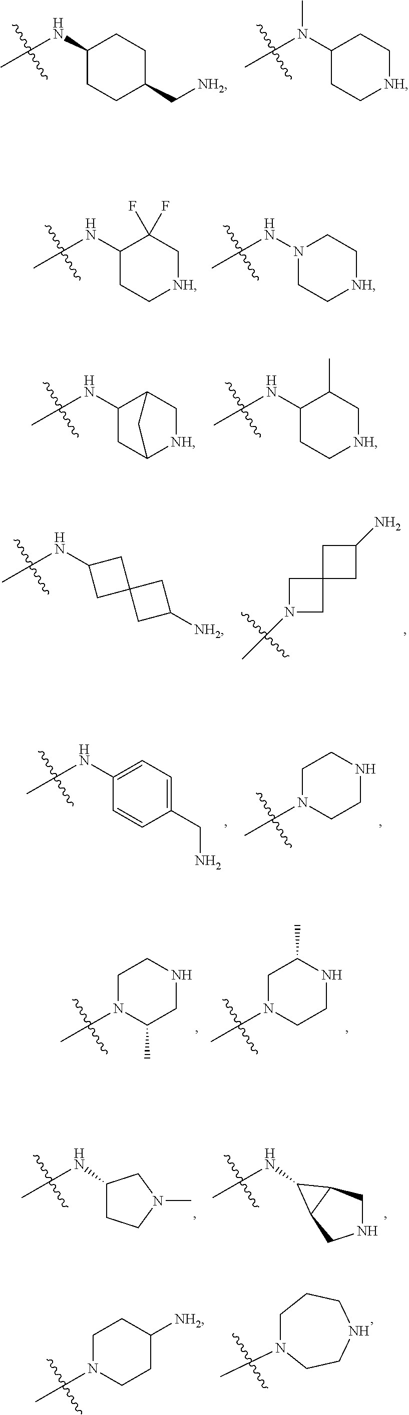 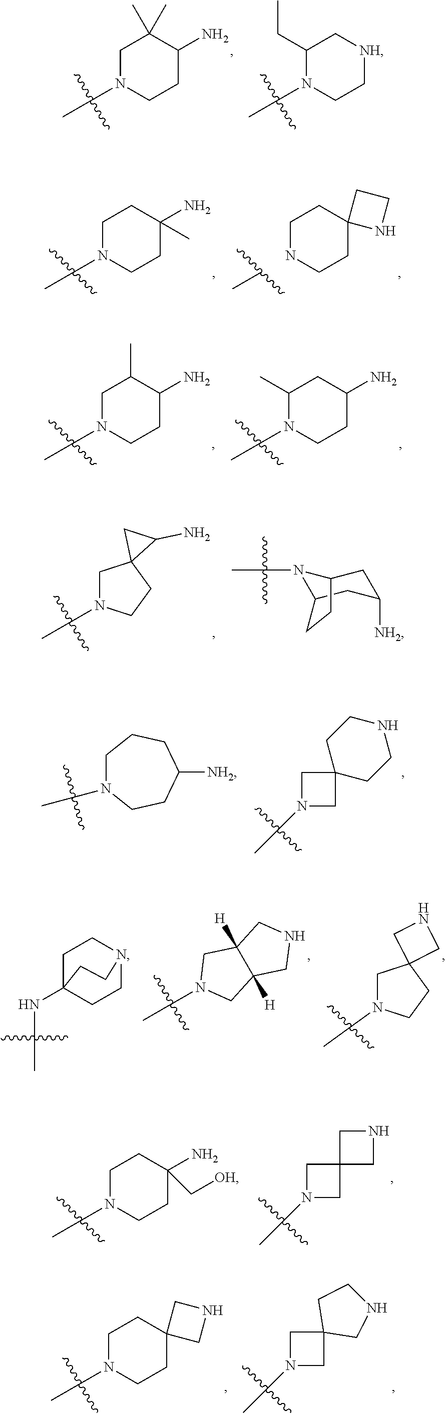 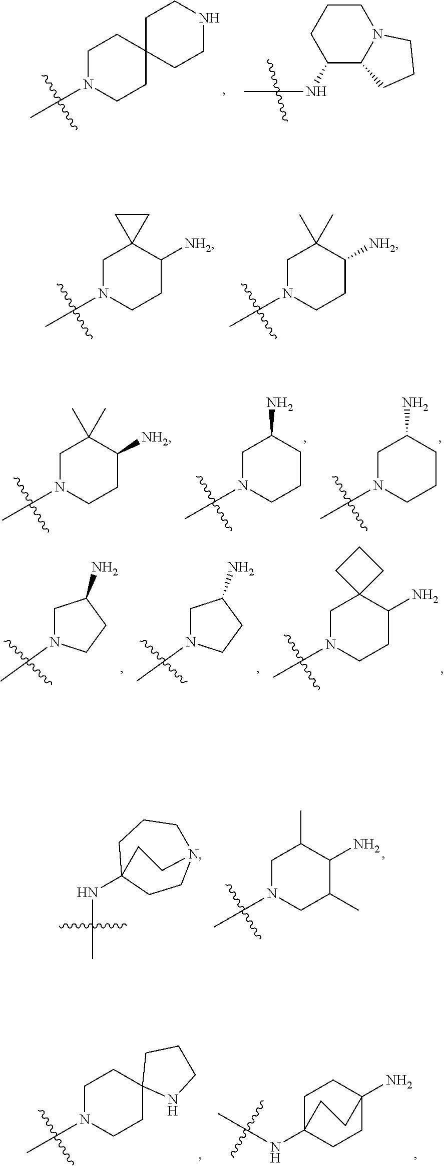 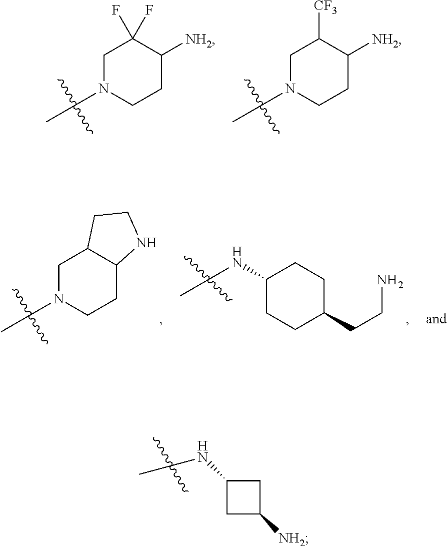 [SMILES: *N1CCC(N)C(F)(F)C1 ; *N1CCC(N)C(C(F)(F)F)C1 ; *N1CCC2NCCC2C1 ; *N[C@H]1CC[C@H](CCN)CC1 ; *N[C@H]1C[C@H](N)C1]
and when X is 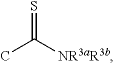
Y is CH₂, and Q is CH₂; or
X is CH, Y is 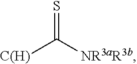
and Q is CH₂; or
X is 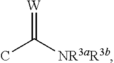
Y is CH₂, W is O or S, and Q is CR²³R²⁴, then
R¹ is selected from (C₆ to C₁₀) aryl and (C₂ to C₉) heteroaryl, wherein
each of the said (C₆ to C₁₀) aryl and (C₂ to C₉) heteroaryl is optionally substituted with at least one R⁸ group;
and when Q is CH₂, then
R² is selected from hydrogen, halogen, OH, nitro, CF₃, —NR^{6a}R^{6b}, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, —C(O)R⁷, —C(O)NR^{6a}R^{6b}, —S(O)ₘR⁷, —S(O)ₘNR^{6a}R^{6b}, —NR^{6a}S(O)ₘR⁷, —(CH₂)ₙC(O)OR⁷, —(CH₂)ₙC(O)N(R^{6a}R^{6b}), —(CH₂)ₙN(R^{6a}R^{6b}), —OC(O)R⁷, —NR^{6a}C(O)R⁷, and —NR^{6a}C(O)N(R^{6a}R^{6b}), wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁸ group;
and when Q is CR²³R²⁴ then R² is hydrogen;
NR^{3a}R^{3b} is selected from the group consisting of 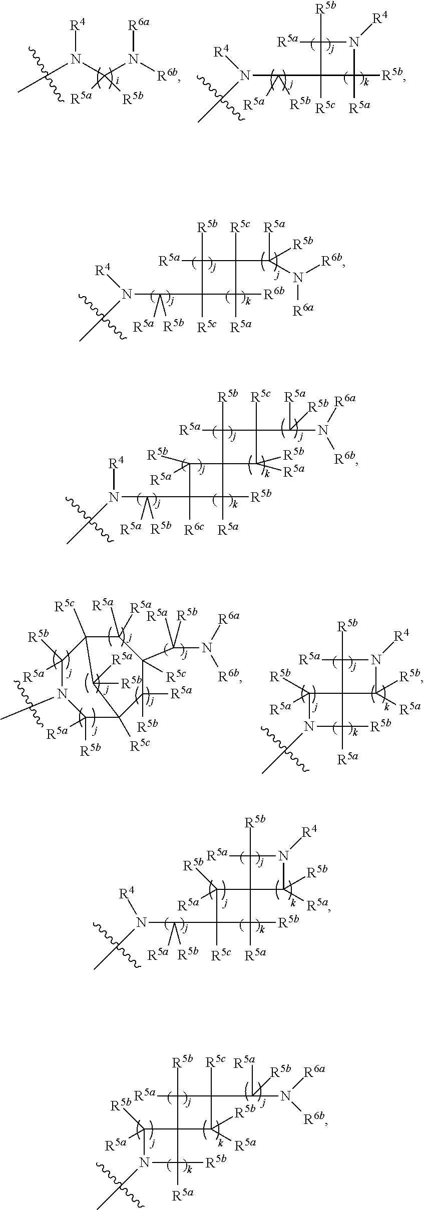 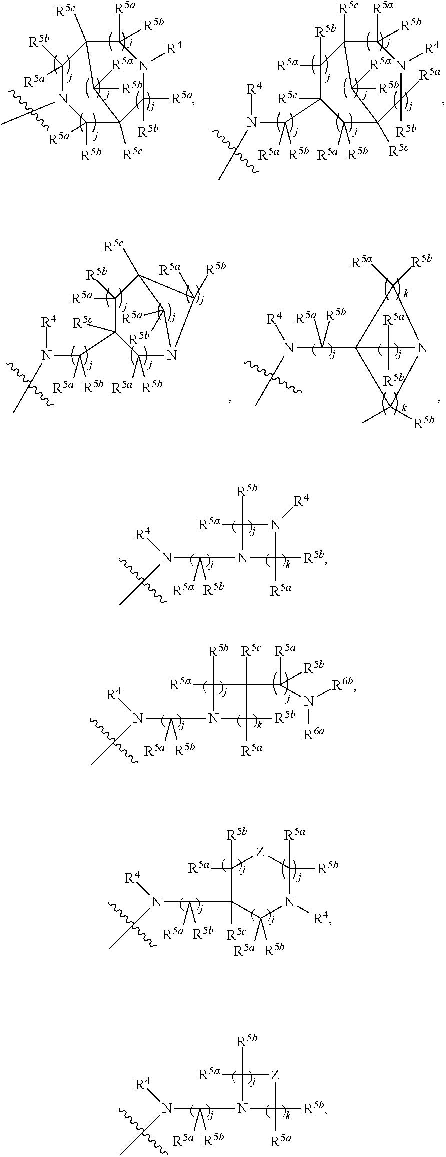 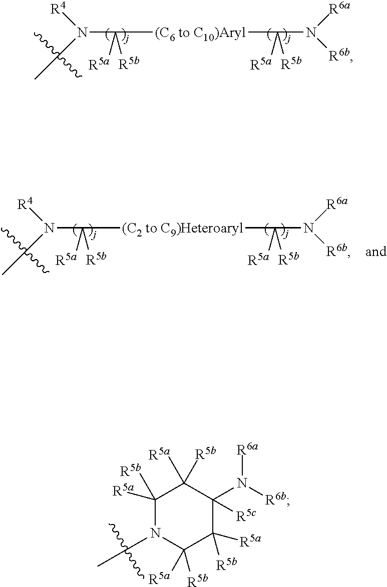
Z is selected from the group consisting of —O—, —S—, —S(O)—, and —S(O)₂—;
each R⁴ is independently selected from hydrogen, (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C to C₁₀) cycloalkenyl, (C₂ to C₉) cycloheteroalkyl, (C₆ to C₁₀) aryl, (C₂ to C₉) heteroaryl, (C₆ to C₁₀) arylene, and (C₂ to C₉) heteroarylene, wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C to C₁₀) cycloalkenyl, (C₂ to C₉) cycloheteroalkyl, (C₆ to C₁₀) aryl, (C₂ to C₉) heteroaryl, (C₆ to C₁₀) arylene, and (C₂ to C₉) heteroarylene is optionally substituted with at least one R⁸ group;
each of the R^{5a}, R^{5b}, and R^{5c} is independently selected from hydrogen, halogen, OH, nitro, CF₃, —NR^{6a}R^{6b}, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, —C(O)R⁷, —C(O)NR^{6a}R^{6b}, —S(O)ₘR⁷, —S(O)ₘNR^{6a}R^{6b}, —NR^{6a}S(O)ₘR⁷, —(CH₂)ₙC(O)OR⁷, —(CH₂)ₙC(O)N(R^{6a}R^{6b}), —(CH₂)ₙN(R^{6a}R^{6b}), —OC(O)R⁷, —NR^{6a}C(O)R⁷, and —NR^{6a}C(O)N(R^{6a}R^{6b}), wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁸ group;
or R^{5a} and R^{5b} may be taken together with the carbon atom to which they are attached to form a (C₃ to C₁₀) cycloalkyl ring, wherein
the said (C₃ to C₁₀) cycloalkyl ring is optionally substituted with at least one R⁸ group;
each of the R^{6a} and R^{6b} is independently selected from hydrogen, (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, (C₆ to C₁₀) arylene, and (C₂ to C₉) heteroarylene, wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, (C₆ to C₁₀) arylene, and (C₂ to C₉) heteroarylene is optionally substituted with at least one R⁸ group,
or R^{6a} and R^{6b} may be taken together with the nitrogen atom to which they are attached to form a (C₂ to C₁₀) cycloheteroalkyl ring, wherein
said (C₂ to C₁₀) cycloheteroalkyl ring has 1 to 3 ring heteroatoms selected from the group consisting of N, O, and S, and wherein
the said (C₂ to C₁₀) cycloheteroalkyl ring is optionally substituted with at least one R⁸ group;
each of the R⁷ is independently selected from hydrogen, (C₁ to C₁₀) alkyl, (C₂ to C₁₀) alkenyl, (C₂ to C₁₀) alkynyl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl, wherein
each of the said (C₁ to C₁₀) alkyl, (C₂ to C₁₀) alkenyl, (C₂ to C₁₀) alkynyl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁸ group;
each R⁸ is independently selected from hydrogen, halogen, OH, nitro, CF₃, —NR^{9a}R^{9b}, oxo, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, (C₆ to C₁₀) arylene, (C₂ to C₉) heteroarylene, (C₃ to C₁₀) cycloalkylene, (C₂ to C₁₀) cycloheteroalkylene, —C(O)R¹⁰, —C(O)NR^{9a}R^{9b}, —S(O)ₘR¹⁰, —S(O)ₘNR^{9a}R^{9b}, —NR^{9a}S(O)ₘR¹⁰, —(CH₂)ₙC(O)OR¹⁰, —(CH₂)ₙC(O)N(R^{9a}R^{9b}), —(CH₂)ₙN(R^{9a}R^{9b}), —OC(O)R¹⁵, —O(CH₂)ₙO—, —NR^{9a}C(O)R¹⁰, and —NR^{9a}C(O)N(R^{9a}R^{9b}), wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, (C₂ to C₉) heteroarylene, (C₃ to C₁₀) cycloalkylene, and (C₂ to C₁₀) cycloheteroalkylene is optionally substituted with at least one R¹¹ group;
each of the R^{9a} and R^{9b} is independently selected from hydrogen, (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, (C₆ to C₁₀) arylene, and (C₂ to C₉) heteroarylene, wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, and (C₂ to C₉) heteroarylene is optionally substituted with at least one R¹¹ group,
or R^{9a} and R^{9b} may be taken together with the nitrogen atom to which they are attached to form a (C₂ to C₁₀) cycloheteroalkyl ring, wherein
said (C₂ to C₁₀) cycloheteroalkyl ring has 1 to 3 ring heteroatoms selected from the group consisting of N, O, and S, and wherein
the said (C₂ to C₁₀) cycloheteroalkyl ring is optionally substituted with at least one R¹¹ group;
each R¹⁰ is independently selected from hydrogen, (C₁ to C₁₀) alkyl, (C₂ to C₁₀) alkenyl, (C₂ to C₁₀) alkynyl, (C₃ to C₁₀) cycloalkyl, (C₅ to C₁₀) cycloalkenyl, (C₂ to C₁₀) cycloheteroalkyl, (C₆ to C₁₀) aryl, and (C₂ to C₉) heteroaryl, wherein
each of the said (C₁ to C₁₀) alkyl, (C₂ to C₁₀) alkenyl, (C₂ to C₁₀) alkynyl, (C₃ to C₁₀) cycloalkyl, (C₅ to C₁₀) cycloalkenyl, (C₂ to C₁₀) cycloheteroalkyl, (C₆ to C₁₀) aryl, and (C₂ to C₉) heteroaryl is optionally substituted with at least one R¹¹ group;
each R¹¹ is independently selected from hydrogen, halogen, OH, nitro, CF₃, —NR^{12a}R^{12b}, oxo, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, (C₂ to C₉) heteroarylene, (C₃ to C₁₀) cycloalkylene, (C₂ to C₁₀) cycloheteroalkylene, —C(O)R¹⁸, —C(O)NR^{12a}R^{12b}, —S(O)ₘR¹³, —S(O)ₘNR^{12a}R^{12b}, —NR^{12a}S(O)ₘR¹³, —(CH₂)ₙC(O)OR¹³, —(CH₂)ₙC(O)N(R^{12a}R^{12b}), —(CH₂)ₙN(R^{12a}R^{12b}), —OC(O)R¹³, —NR^{12a}C(O)R¹³, and —NR^{12a}C(O)N(R^{12a}R^{12b}) wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₂ to C₉) heteroaryl, (C₆ to C₁₀) aryl, (C₆ to C₁₀) arylene, (C₂ to C₉) heteroarylene, (C₃ to C₁₀) cycloalkylene, and (C₂ to C₁₀) cycloheteroalkylene is optionally substituted with at least one R¹⁴ group;
each of the R^{12a} and R^{12b} is independently selected from hydrogen, (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, and (C₂ to C₉) heteroarylene, wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, and (C₂ to C₉) heteroarylene is optionally substituted with at least one R¹⁴ group,
or R^{12a} and R^{12b} may be taken together with the nitrogen atom to which they are attached to form a (C₂ to C₁₀) cycloheteroalkyl ring, wherein
said (C₂ to C₁₀) cycloheteroalkyl ring has 1 to 3 ring heteroatoms selected from the group consisting of N, O, and S, and wherein
the said (C₂ to C₁₀) cycloheteroalkyl ring is optionally substituted with at least one R¹⁴ group;
each R¹³ is independently selected from hydrogen, halogen, OH, nitro, CF₃, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C to C₁₀) cycloalkenyl, (C₂ to C₁₀) cycloheteroalkyl, (C₆ to C₁₀) aryl, and (C₂ to C₉) heteroaryl, wherein
each of the said (C₁ to C₁₀) alkyl, (C₂ to C₁₀) alkenyl, (C₂ to C₁₀) alkynyl, (C₃ to C₁₀) cycloalkyl, (C₅ to C₁₀) cycloalkenyl, (C₂ to C₁₀) cycloheteroalkyl, (C₂ to C₉) heteroaryl, and (C₆ to C₁₀) aryl is optionally substituted with at least one R¹⁴ group;
each R¹⁴ is independently selected from hydrogen, halogen, OH, nitro, CF₃, —NR^{15a}R^{15b}, oxo, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, (C₂ to C₉) heteroarylene, (C₃ to C₁₀) cycloalkylene, (C₂ to C₁₀) cycloheteroalkylene, —C(O)R¹⁶, —C(O)NR^{15a}R^{15b}, —S(O)ₘR¹⁶, —S(O)ₘNR^{15a}R^{15b}, —NR^{15a}S(O)ₘR¹⁶, —(CH₂)ₙC(O)OR¹⁶, —(CH₂)ₙC(O)N(R^{15a}R^{15b}), —(CH₂)ₙN(R^{15a}R^{15b}), —OC(O)R¹⁶, —NR^{15a}C(O)R¹⁶, and —NR^{15a}C(O)N(R^{15a}R^{15b}) wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, (C₂ to C₉) heteroarylene, (C₃ to C₁₀) cycloalkylene, and (C₂ to C₁₀) cycloheteroalkylene is optionally substituted with at least one R¹⁷ group;
each of the R^{15a} and R^{15b} is independently selected from hydrogen, (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, and (C₂ to C₉) heteroaryl, wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, and (C₂ to C₉) heteroaryl is optionally substituted with at least one R¹⁷ group,
or R^{15a} and R^{15b} may be taken together with the nitrogen atom to which they are attached to form a (C₂ to C₁₀) cycloheteroalkyl ring, wherein
said (C₂ to C₁₀) cycloheteroalkyl ring has 1 to 3 ring heteroatoms selected from the group consisting of N, O, and S, and wherein
the said (C₂ to C₁₀) cycloheteroalkyl ring is optionally substituted with at least one R¹⁷ group;
each R¹⁶ is independently selected from hydrogen, halogen, OH, nitro, CF₃, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₁₀) cycloheteroalkyl, (C₆ to C₁₀) aryl, and (C₂ to C₉) heteroaryl;
each R¹⁷ is independently selected from hydrogen, halogen, OH, nitro, CF₃, —NR^{18a}R^{18b}, oxo, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, (C₂ to C₉) heteroarylene, (C₃ to C₁₀) cycloalkylene, (C₂ to C₁₀) cycloheteroalkylene, —C(O)R¹⁹, —C(O)NR^{18a}R^{18b}, —S(O)ₘR¹⁹, —S(O)ₘNR^{18a}R^{18b}, —NR^{18a}S(O)ₘR¹⁹, —(CH₂)ₙC(O)OR¹⁹, —(CH₂)ₙC(O)N(R^{18a}R^{18b}), —(CH₂)ₙN(R^{18a}R^{18b}), —OC(O)R¹⁹, —NR^{18a}C(O)R¹⁹, and —NR^{18a}C(O)N(R^{18a}R^{18b}) wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C to C₁₀) arylene, (C₂ to C₉) heteroarylene, (C₃ to C₁₀) cycloalkylene, and (C₂ to C₁₀) cycloheteroalkylene is optionally substituted with at least one R²⁰ group;
each of the R^{18a} and R^{18b} is independently selected from hydrogen, (C₁ to C₁₀) alkyl, (C₂ to C₁₀) alkenyl, (C₂ to C₁₀) alkynyl, (C₃ to C₁₀) cycloalkyl, (C₅ to C₁₀) cycloalkenyl, (C₂ to C₁₀) cycloheteroalkyl, (C₂ to C₉) heteroaryl, and (C to C₁₀) aryl;
each R¹⁹ is independently selected from hydrogen, halogen, OH, nitro, CF₃, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C to C₁₀) cycloalkenyl, (C₂ to C₁₀) cycloheteroalkyl, (Ceto C₁₀) aryl, (C₂ to C₉) heteroaryl;
each R²⁰ is independently selected from hydrogen, halogen, OH, nitro, CF₃, —NR^{21a}R^{21b}, oxo, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, (C₂ to C₉) heteroarylene, (C₃ to C₁₀) cycloalkylene, (C₂ to C₁₀) cycloheteroalkylene, —C(O)R²², —C(O)NR^{21a}R^{21b}, —S(O)ₘR²², —S(O)ₘNR^{21a}R^{21b}, —NR^{21a}S(O)ₘR²², —(CH₂)ₙC(O)OR²², —(CH₂)ₙC(O)N(R^{21a}R^{21b}), —(CH₂)ₙN(R^{21a}R^{21b}), —OC(O)R²², —NR^{21a}C(O)R²², and —NR^{21a}C(O)N(R^{21a}R^{21b}) wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, (C₂ to C₉) heteroarylene, (C₃ to C₁₀) cycloalkylene, and (C₂ to C₁₀) cycloheteroalkylene is optionally substituted with at least one R²² group;
each of the R^{21a} and R^{21b} is independently selected from hydrogen, (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₂ to C₁₀) cycloalkenyl, (C₂ to C₁₀) cycloheteroalkyl, (C₆ to C₁₀) aryl, and (C₂ to C₉) heteroaryl,
or R^{21a} and R^{21b} may be taken together with the nitrogen atom to which they are attached to form a (C₂ to C₁₀) cycloheteroalkyl ring, wherein
said (C₂ to C₁₀) cycloheteroalkyl ring has 1 to 3 ring heteroatoms selected from the group consisting of N, O, and S;
each R²² is independently selected from hydrogen, halogen, OH, nitro, CF₃, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₁₀) cycloheteroalkyl, (Ceto C₁₀) aryl, (C₂ to C₉) heteroaryl;
each R²³ is independently selected from hydrogen, halogen, OH, nitro, CF₃, —NR^{11a}R^{11b}, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cyclo-alkenyl, (C₂ to C₉) cycloheteroalkyl, (C₆ to C₁₀) aryl, (C₂ to C₉) heteroaryl, —C(O)R¹², —C(O)NR^{11a}R^{11b}, —S(O)ₘR¹², —S(O)ₘNR^{11a}R^{11b}, —NR^{11a}S(O)ₘR¹², —(CH₂)ₙC(O)OR¹², —(CH₂)ₙC(O)N(R^{11a}R^{11b}), —(CH₂)ₙN(R^{11a}R^{11b}), —OC(O)R¹², —NR^{11a}C(O)R¹², and —NR^{11a}C(O)N(R^{11a}R^{11b}) wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₉) cycloheteroalkyl, (C₅ to C₁₀) aryl, and (C₂ to C₉) heteroaryl is optionally substituted with at least one R⁸ group;
each R²⁴ is independently selected from halogen, OH, nitro, CF₃, —NR^{11a}R^{11b}, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cyclo-alkenyl, (C₂ to C₉) cycloheteroalkyl, (C₆ to C₁₀) aryl, (C₂ to C₉) heteroaryl, —C(O)R¹², —C(O)NR^{11a}R^{11b}, —S(O)ₘR¹², —S(O)ₘNR^{11a}R^{11b}, —NR^{11a}S(O)ₘR¹², —(CH₂)ₙC(O)OR¹², —(CH₂)ₙC(O)N(R^{11a}R^{11b}), —(CH₂)ₙN(R^{11a}R^{11b}), —OC(O)R¹², —NR^{11a}C(O)R¹², and —NR^{11a}C(O)N(R^{11a}R^{11b}), wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁸ group;
or R²³ and R²⁴ may be taken together with the carbon atom to which they are attached to form a (C₃ to C₁₀) cycloalkyl or (C₂ to C₉) cycloheteroalkyl ring, wherein
the said (C₃ to C₁₀) cycloalkyl or (C₂ to C₉) cycloheteroalkyl ring is optionally substituted with at least one R⁸ group;
or R²³ and R²⁴ may be taken together with the carbon atom to which they are attached to form a carbonyl or alkene, which is optionally substituted with at least one R⁸ group;
i is 2, 3, 4, 5, or 6;
j is 0, 1, 2, 3, 4, or 5;
k is 1, 2, 3, 4, or 5;
m is 0, 1 or 2;
n is 0, 1, 2, 3, or 4;
with the proviso that when R¹ is phenyl, R² is hydrogen, X is 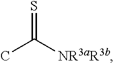
Y is CH₂, and Q is CH₂, then NR^{3a}R^{3b} is not 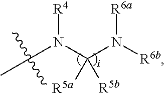
and with the proviso that the following compounds are excluded:

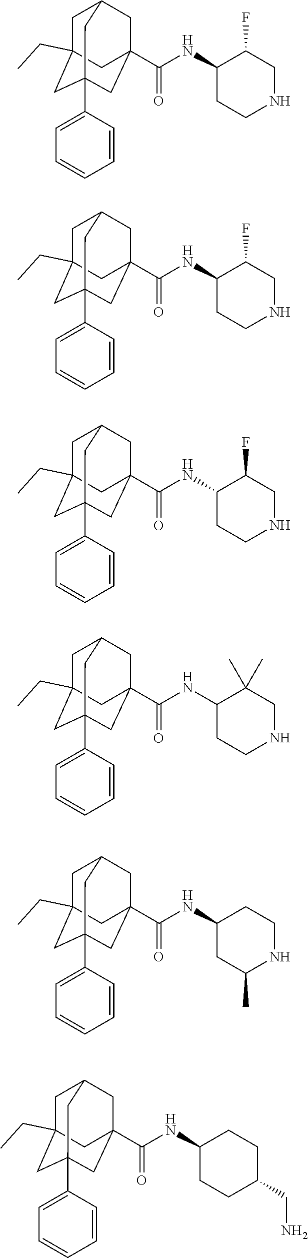

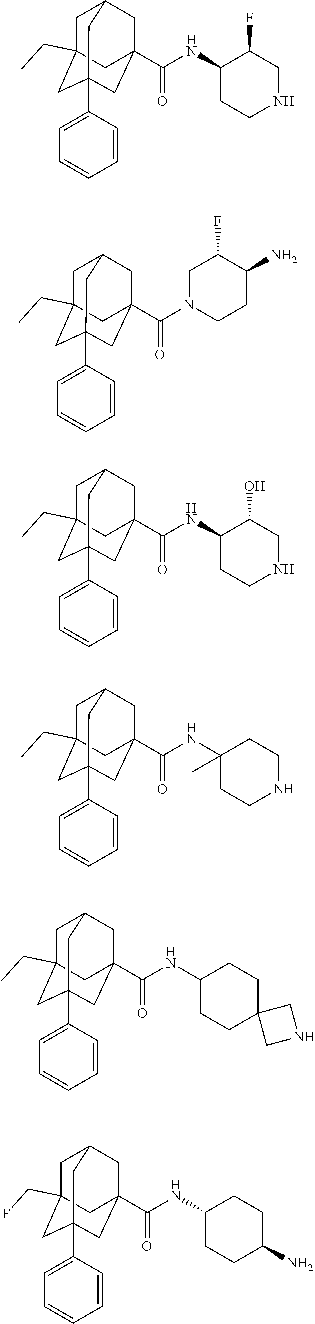

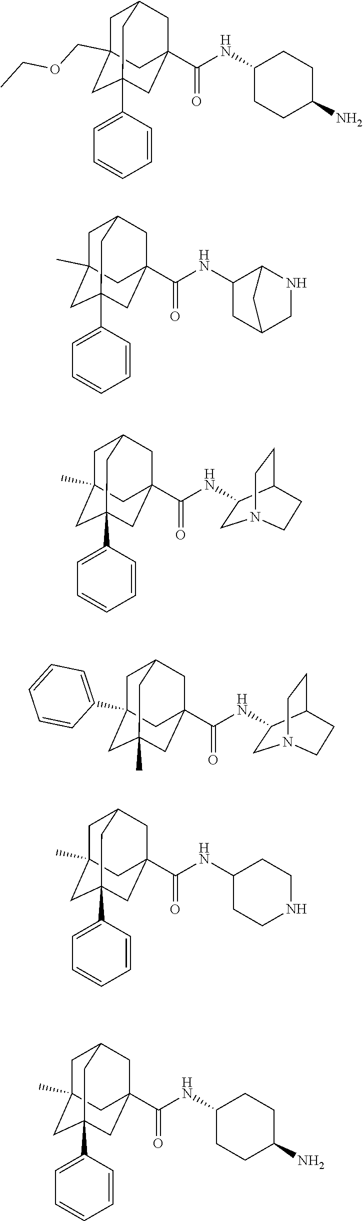

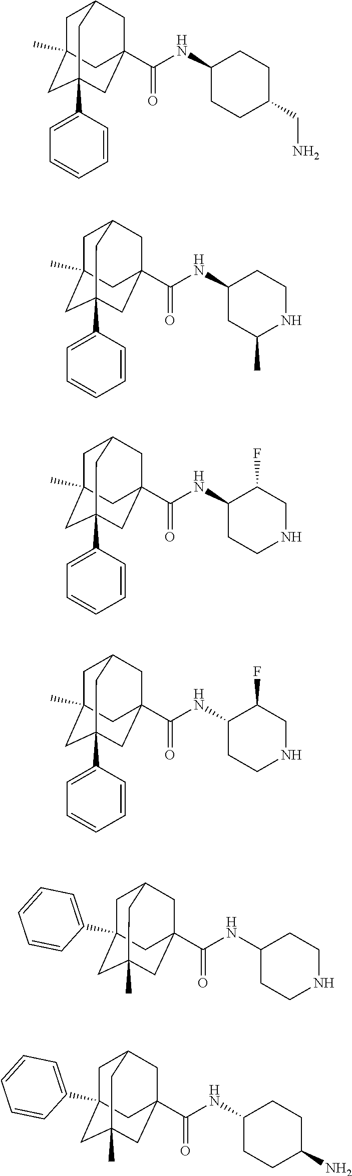

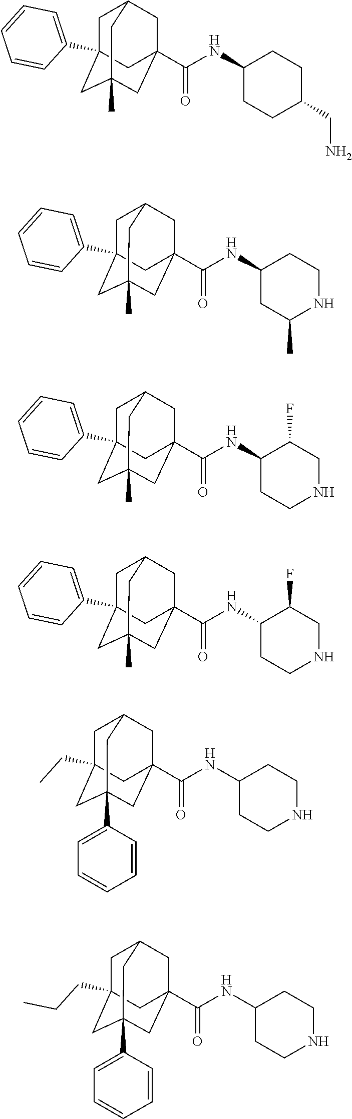 [SMILES: C[C@@]12CC3CC(C(=O)N[C@H]4CC[C@H](CN)CC4)(C1)C[C@](c1ccccc1)(C3)C2 ; C[C@H]1C[C@@H](NC(=O)C23CC4C[C@](C)(C2)C[C@@](c2ccccc2)(C4)C3)CCN1 ; C[C@@]12CC3CC(C(=O)N[C@@H]4CCNC[C@H]4F)(C1)C[C@](c1ccccc1)(C3)C2 ; C[C@@]12CC3CC(C(=O)N[C@H]4CCNC[C@@H]4F)(C1)C[C@](c1ccccc1)(C3)C2 ; CC[C@]12CC3CC(C(=O)NC4CCNCC4)(C1)C[C@@](c1ccccc1)(C3)C2 ; CCC[C@]12CC3CC(C(=O)NC4CCNCC4)(C1)C[C@@](c1ccccc1)(C3)C2]

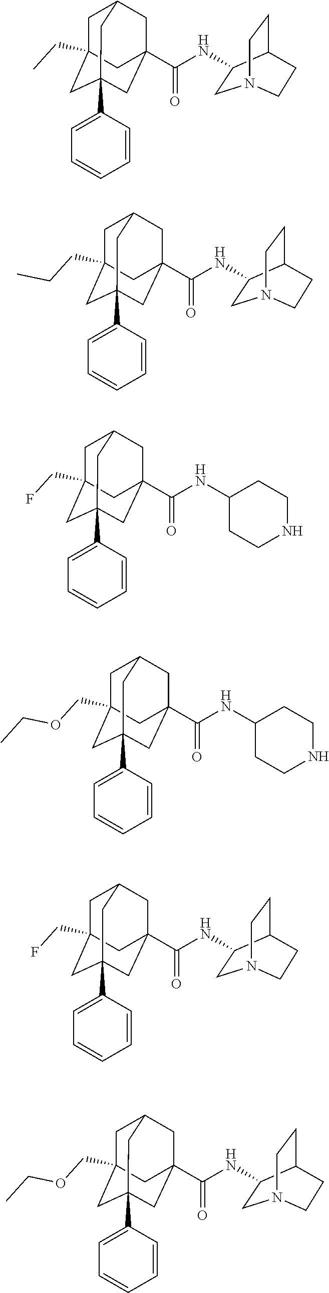

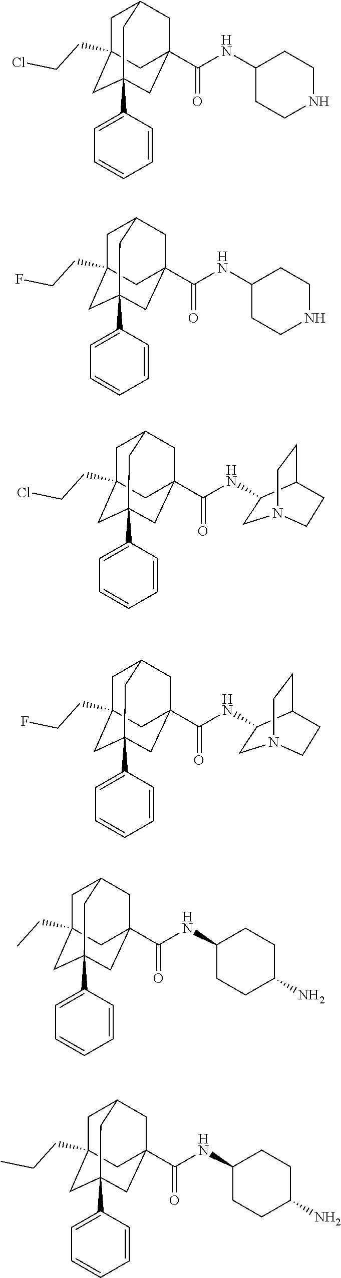

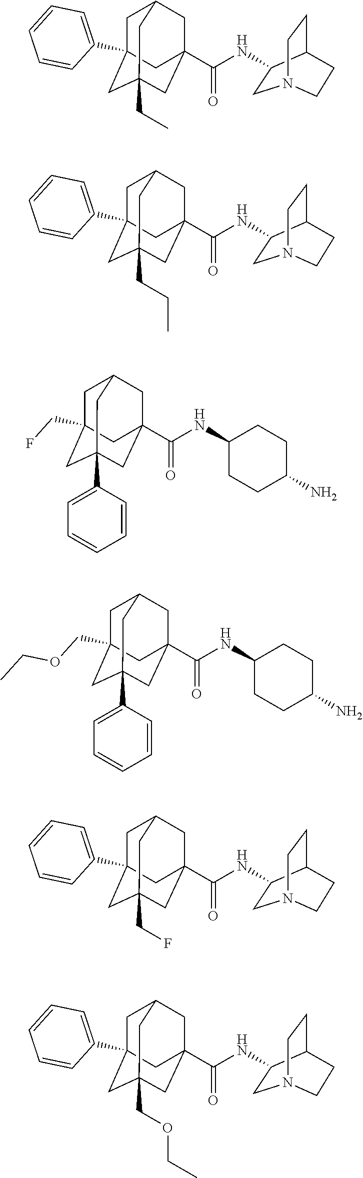

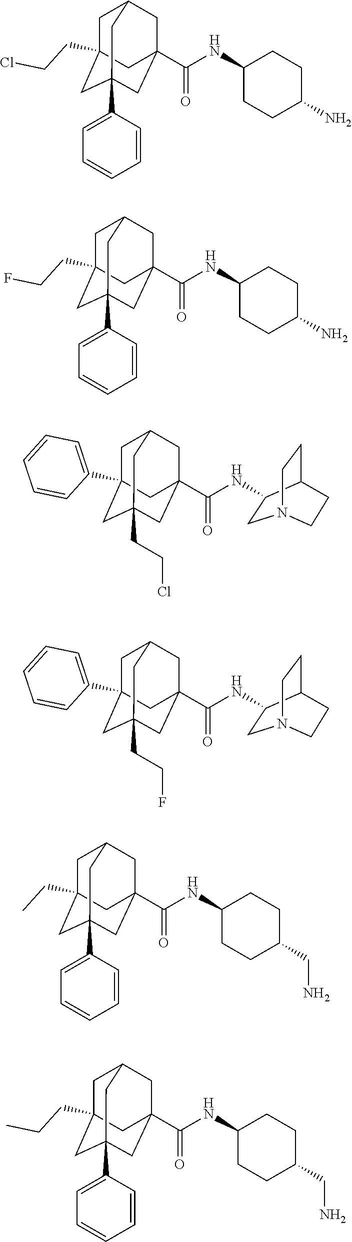

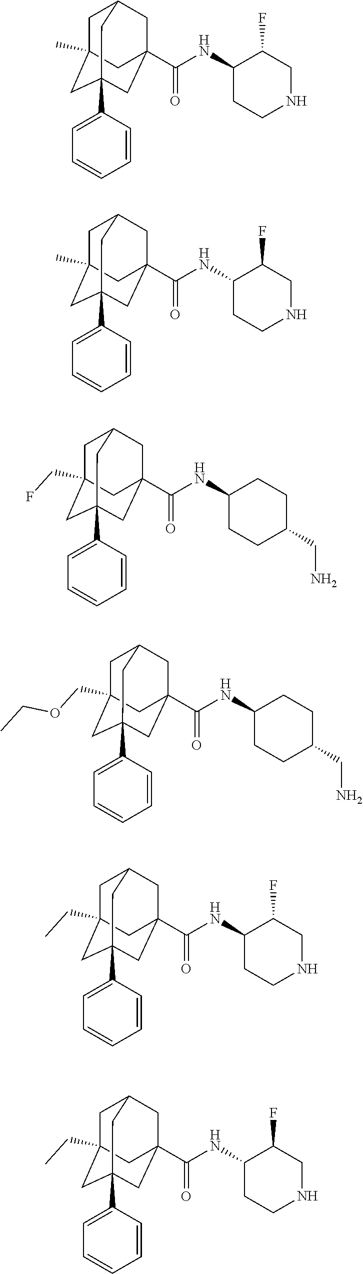

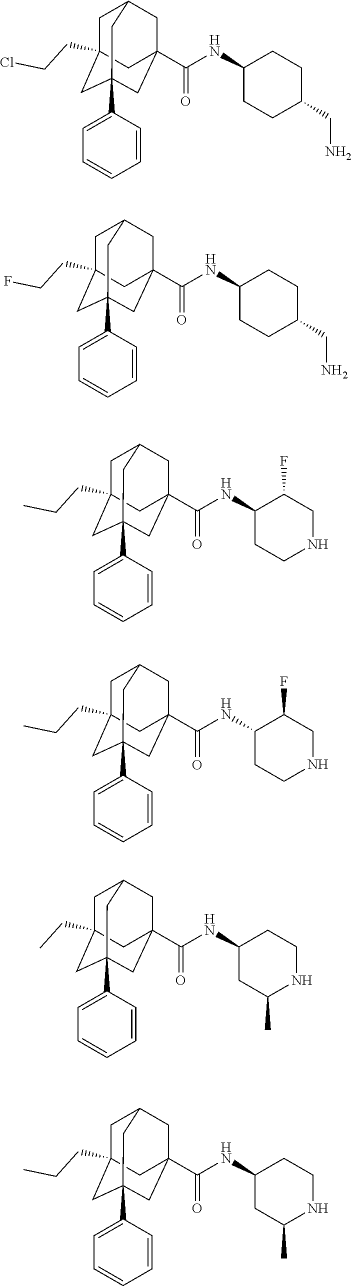

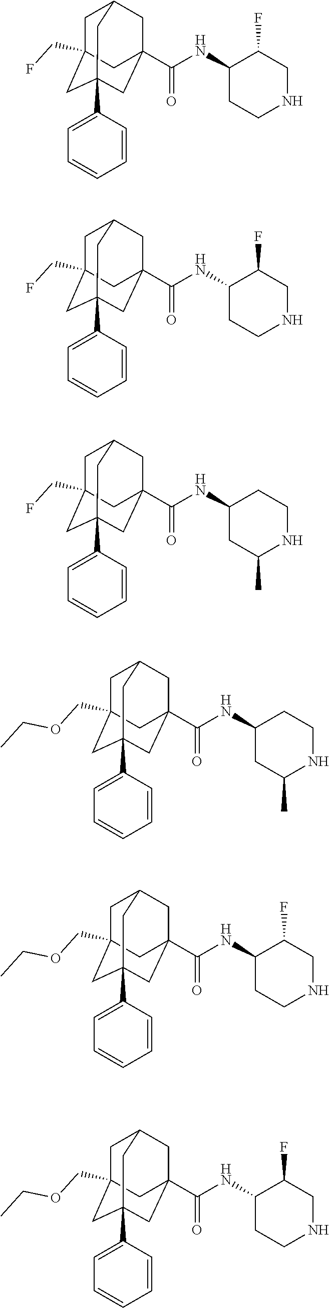

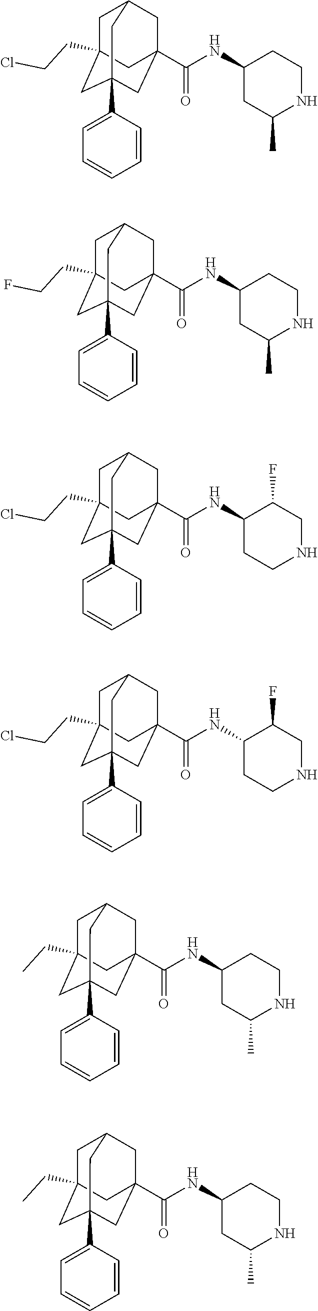 [SMILES: C[C@H]1C[C@@H](NC(=O)C23CC4C[C@@](CCCl)(C2)C[C@](c2ccccc2)(C4)C3)CCN1 ; C[C@H]1C[C@@H](NC(=O)C23CC4C[C@@](CCF)(C2)C[C@](c2ccccc2)(C4)C3)CCN1 ; O=C(N[C@@H]1CCNC[C@H]1F)C12CC3C[C@@](CCCl)(C1)C[C@](c1ccccc1)(C3)C2 ; O=C(N[C@H]1CCNC[C@@H]1F)C12CC3C[C@@](CCCl)(C1)C[C@](c1ccccc1)(C3)C2 ; CC[C@]12CC3CC(C(=O)N[C@H]4CCN[C@H](C)C4)(C1)C[C@@](c1ccccc1)(C3)C2 ; CC[C@]12CC3CC(C(=O)N[C@H]4CCN[C@H](C)C4)(C1)C[C@@](c1ccccc1)(C3)C2]

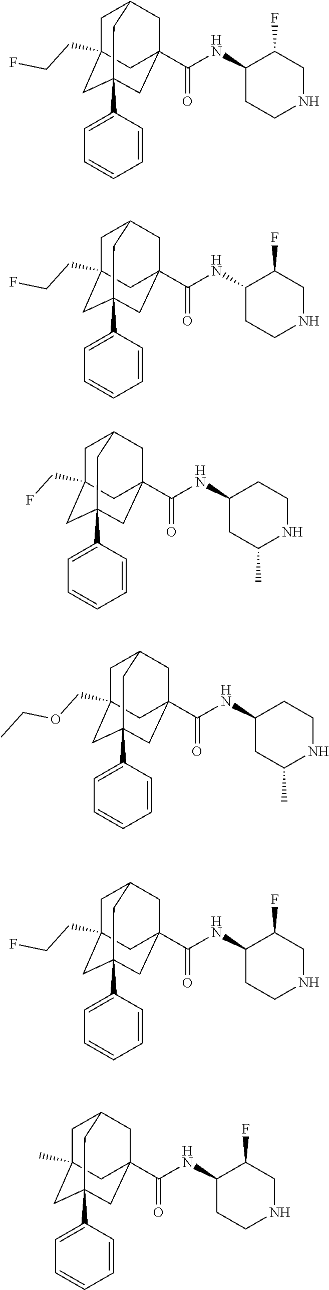

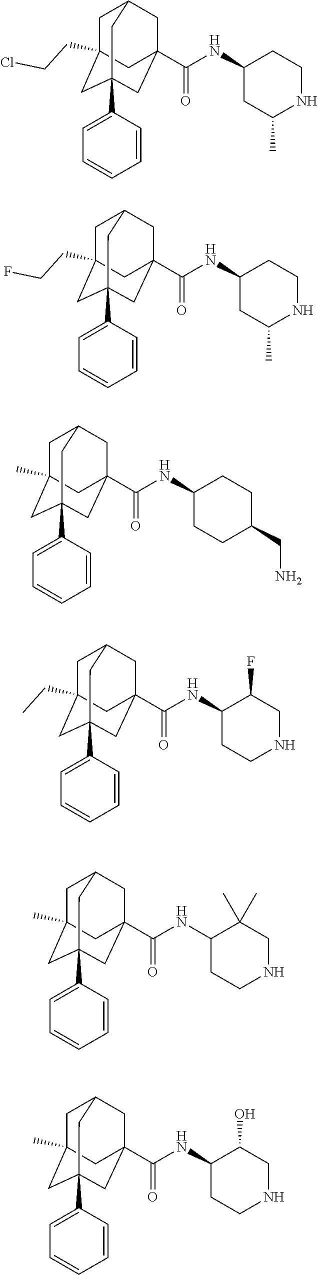

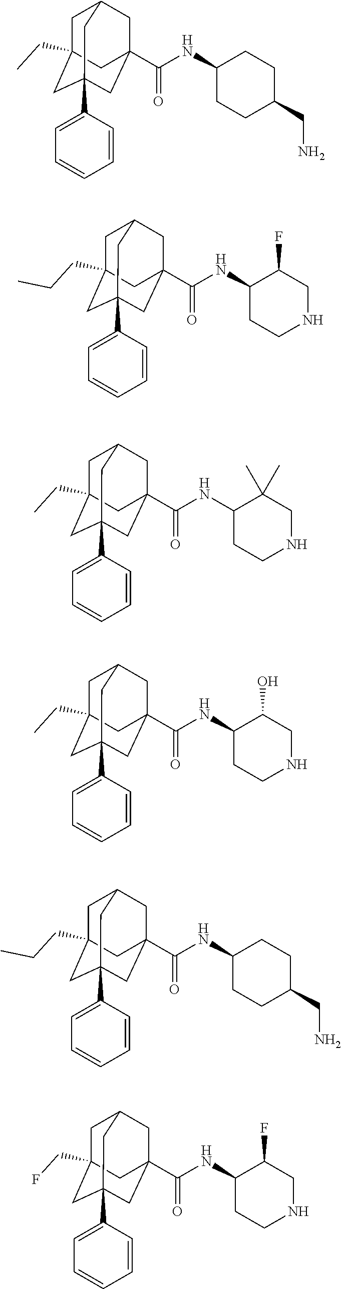

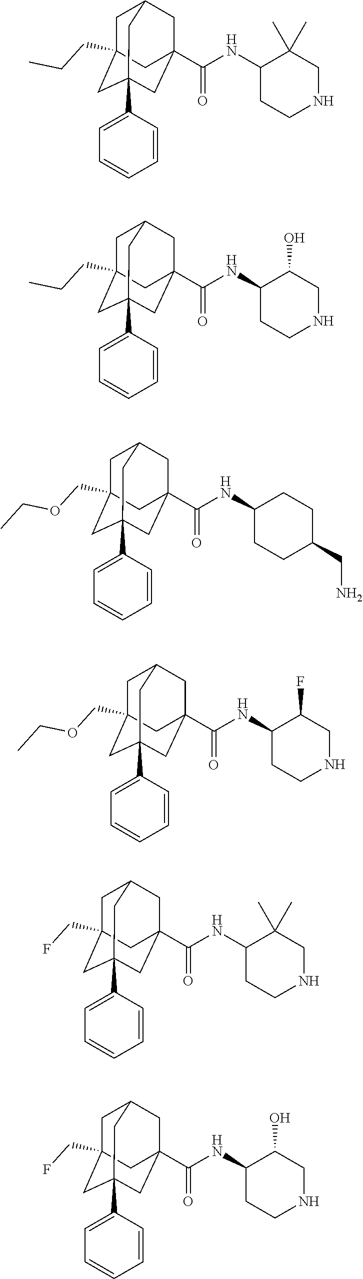

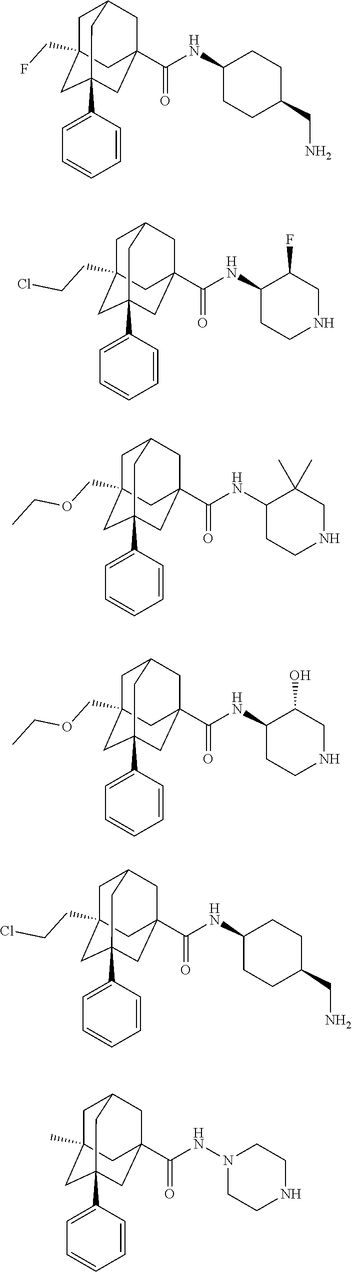

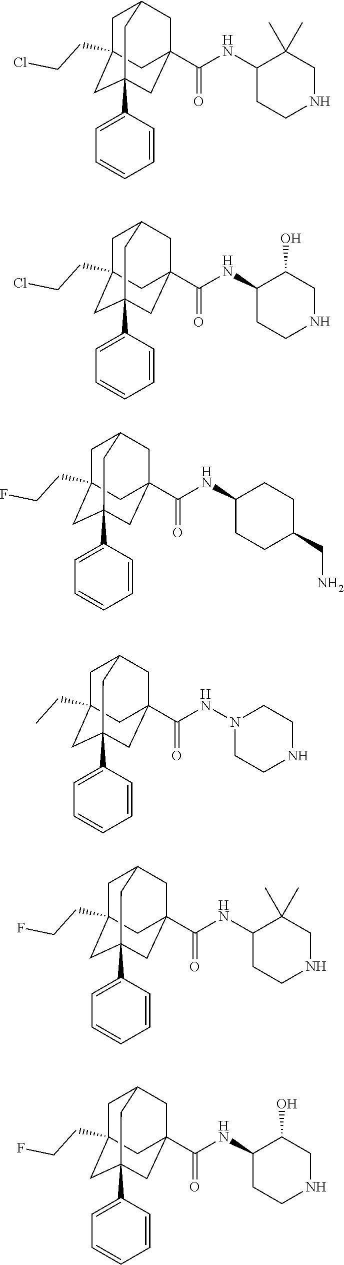

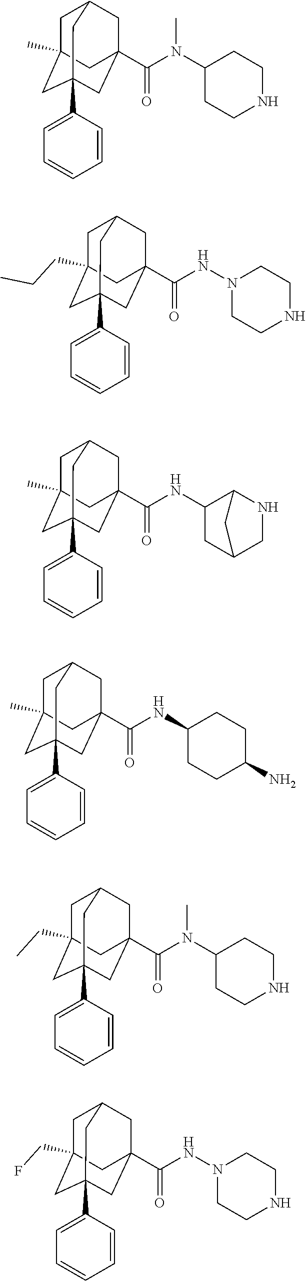

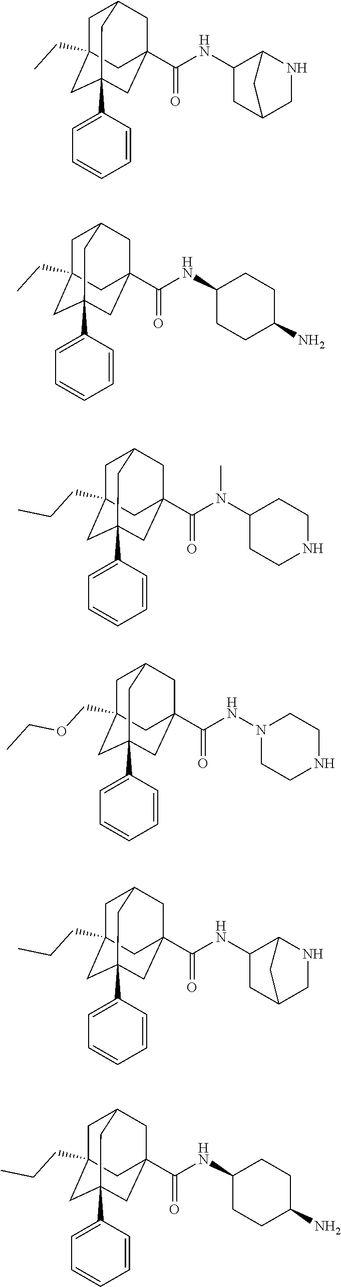

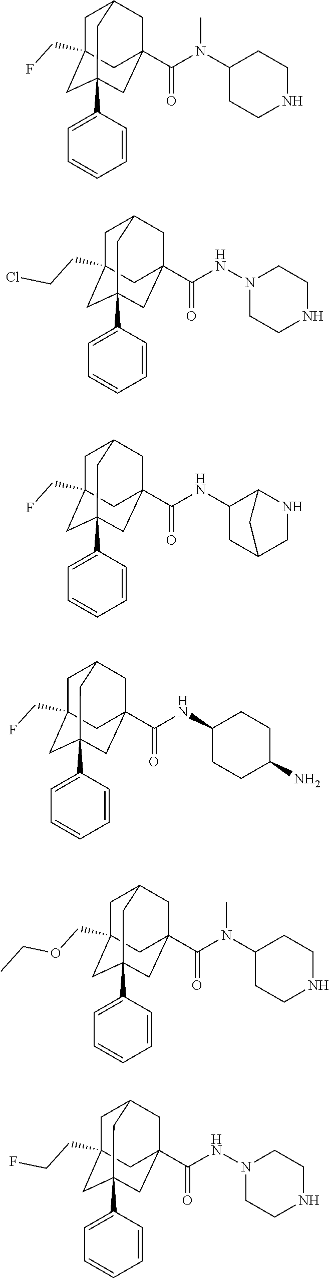

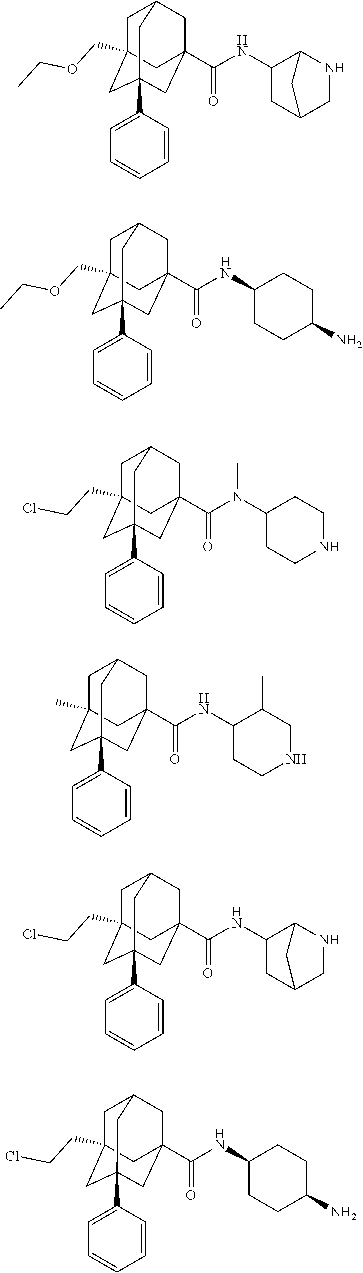

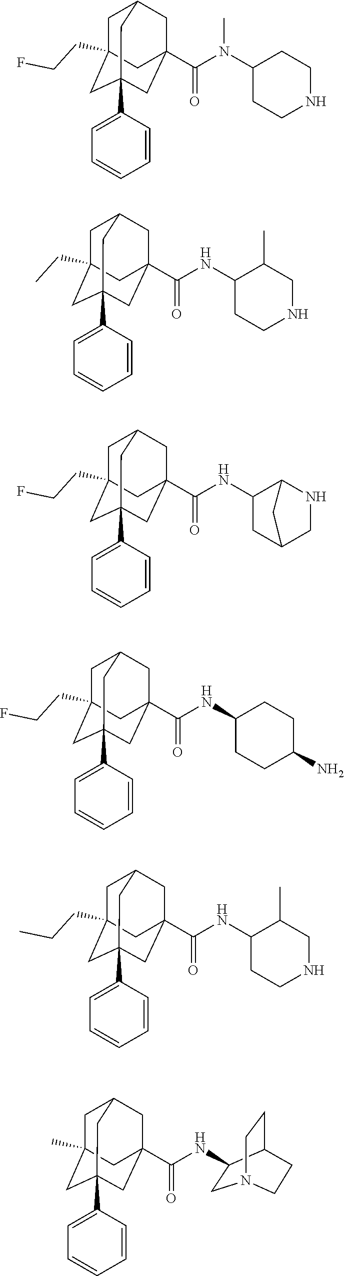

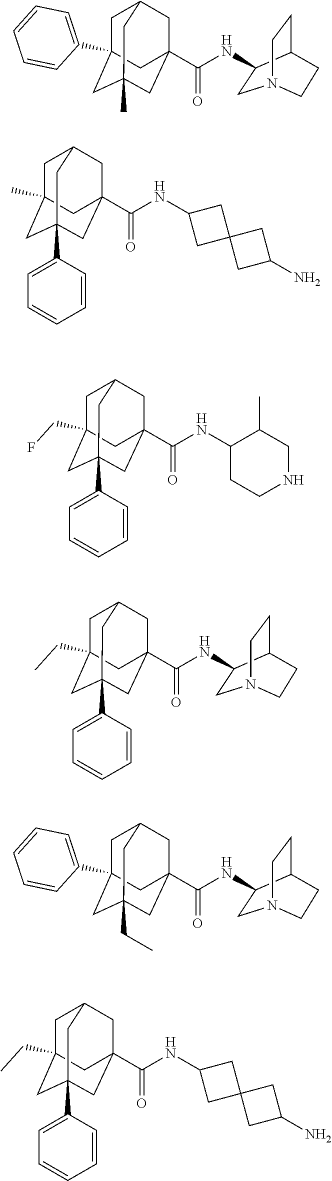 [SMILES: C[C@@]12CC3CC(C(=O)N[C@@H]4CN5CCC4CC5)(C1)C[C@](c1ccccc1)(C3)C2 ; C[C@]12CC3CC(C(=O)NC4CC5(CC(N)C5)C4)(C1)C[C@@](c1ccccc1)(C3)C2 ; CC1CNCCC1NC(=O)C12CC3C[C@@](CF)(C1)C[C@](c1ccccc1)(C3)C2 ; CC[C@]12CC3CC(C(=O)N[C@@H]4CN5CCC4CC5)(C1)C[C@@](c1ccccc1)(C3)C2 ; CC[C@@]12CC3CC(C(=O)N[C@@H]4CN5CCC4CC5)(C1)C[C@](c1ccccc1)(C3)C2 ; CC[C@]12CC3CC(C(=O)NC4CC5(CC(N)C5)C4)(C1)C[C@@](c1ccccc1)(C3)C2]

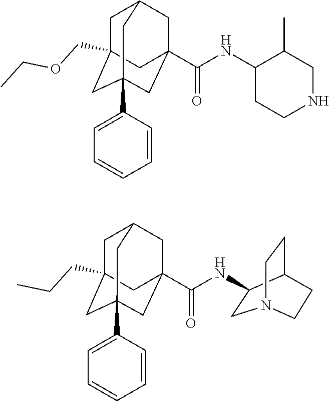

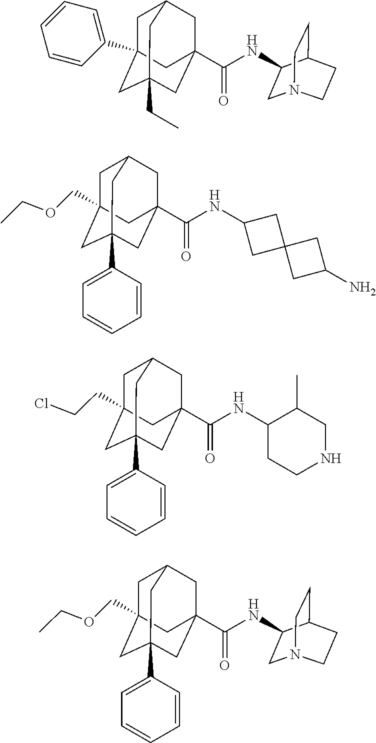

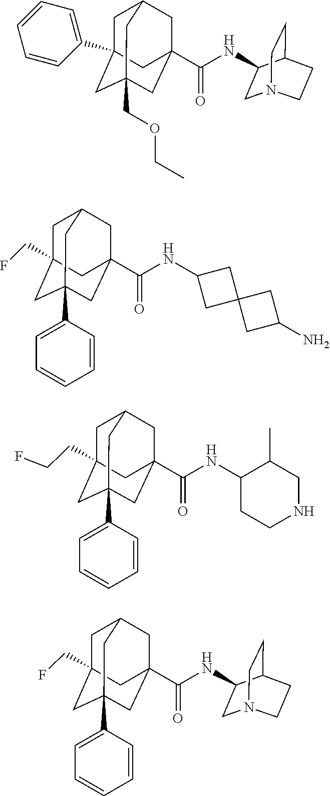

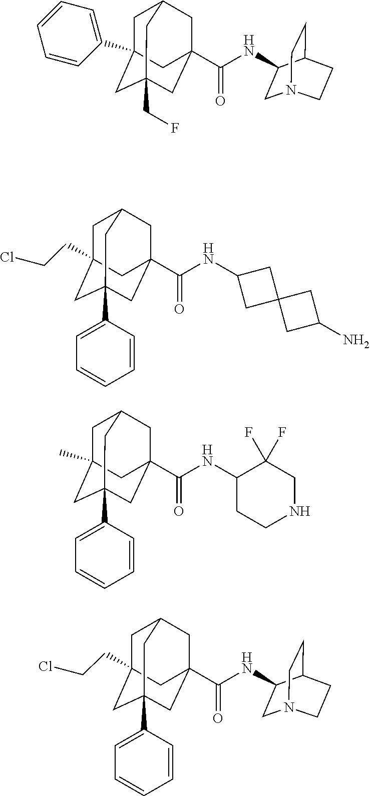

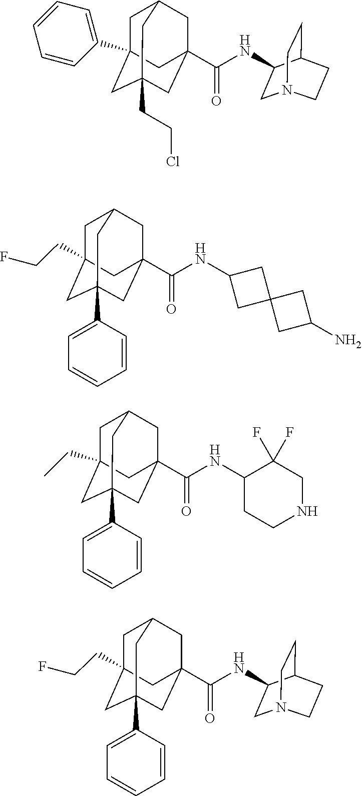

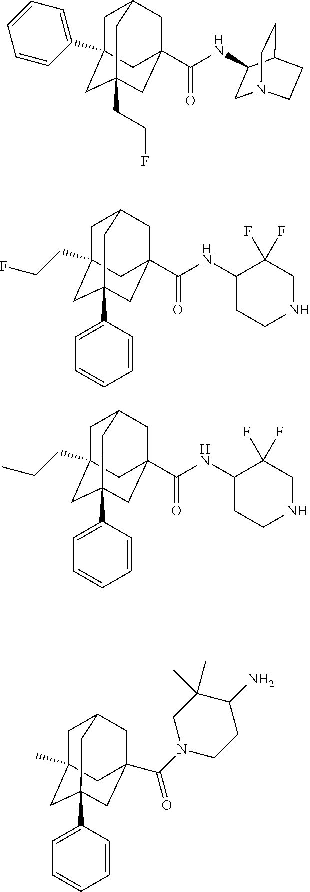

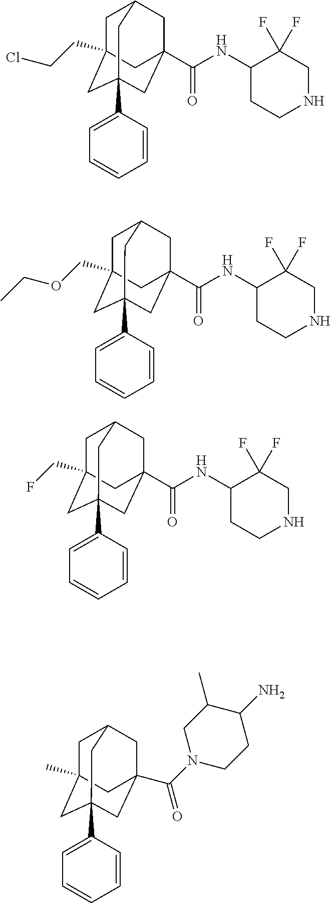

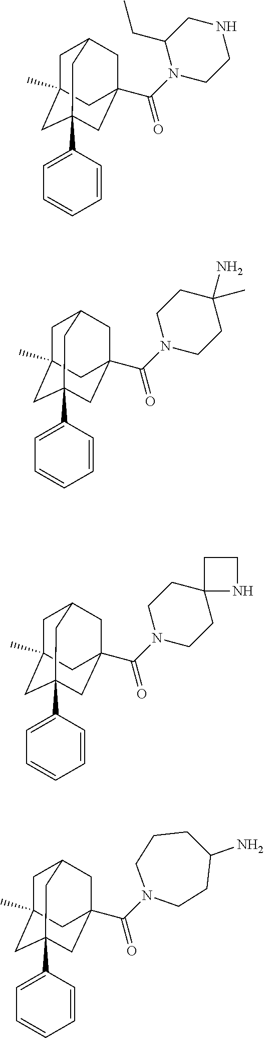

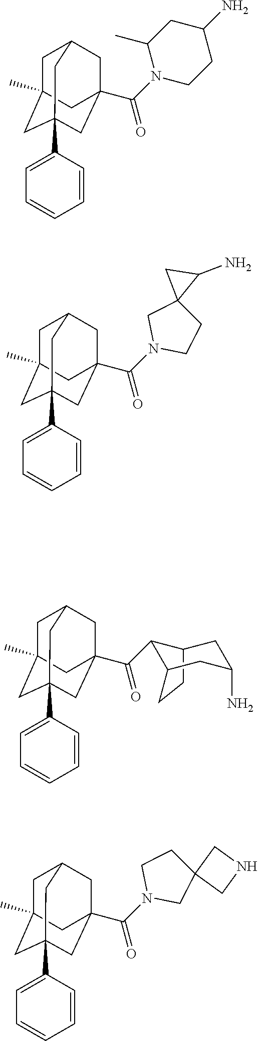 [SMILES: CC1CC(N)CCN1C(=O)C12CC3C[C@@](C)(C1)C[C@](c1ccccc1)(C3)C2 ; C[C@]12CC3CC(C(=O)N4CCC5(CC5N)C4)(C1)C[C@@](c1ccccc1)(C3)C2 ; C[C@]12CC3CC(C(=O)C4C5CCC4CC(N)C5)(C1)C[C@@](c1ccccc1)(C3)C2 ; C[C@]12CC3CC(C(=O)N4CCC5(CNC5)C4)(C1)C[C@@](c1ccccc1)(C3)C2]

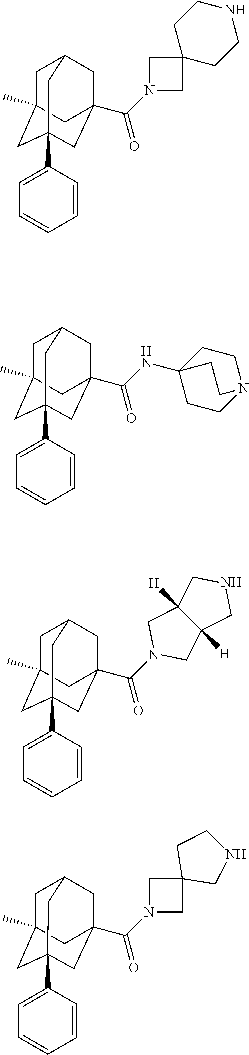 [SMILES: C[C@]12CC3CC(C(=O)N4CC5(CCNCC5)C4)(C1)C[C@@](c1ccccc1)(C3)C2 ; C[C@]12CC3CC(C(=O)NC45CCN(CC4)CC5)(C1)C[C@@](c1ccccc1)(C3)C2 ; C[C@]12CC3CC(C(=O)N4C[C@H]5CNC[C@H]5C4)(C1)C[C@@](c1ccccc1)(C3)C2 ; C[C@]12CC3CC(C(=O)N4CC5(CCNC5)C4)(C1)C[C@@](c1ccccc1)(C3)C2]

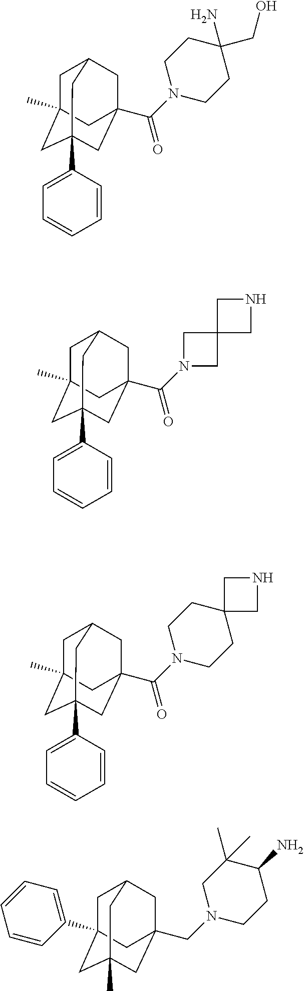 [SMILES: C[C@]12CC3CC(C(=O)N4CCC(N)(CO)CC4)(C1)C[C@@](c1ccccc1)(C3)C2 ; C[C@]12CC3CC(C(=O)N4CC5(CNC5)C4)(C1)C[C@@](c1ccccc1)(C3)C2 ; C[C@]12CC3CC(C(=O)N4CCC5(CC4)CNC5)(C1)C[C@@](c1ccccc1)(C3)C2 ; CC1(C)CN(CC23CC4C[C@](C)(C2)C[C@@](c2ccccc2)(C4)C3)CC[C@@H]1N]

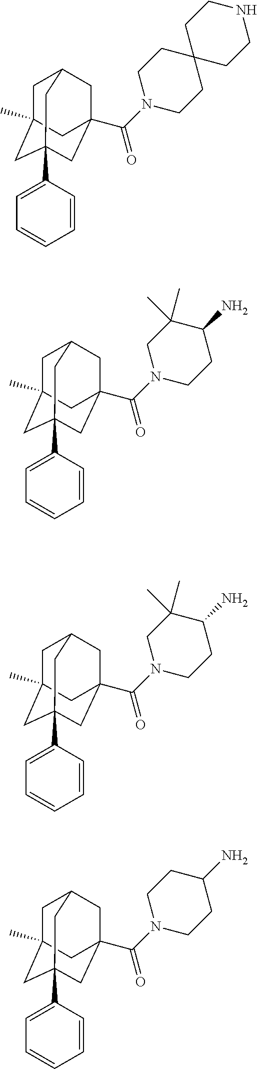 [SMILES: C[C@]12CC3CC(C(=O)N4CCC5(CCNCC5)CC4)(C1)C[C@@](c1ccccc1)(C3)C2 ; CC1(C)CN(C(=O)C23CC4C[C@@](C)(C2)C[C@](c2ccccc2)(C4)C3)CC[C@@H]1N ; CC1(C)CN(C(=O)C23CC4C[C@@](C)(C2)C[C@](c2ccccc2)(C4)C3)CC[C@H]1N ; C[C@]12CC3CC(C(=O)N4CCC(N)CC4)(C1)C[C@@](c1ccccc1)(C3)C2]

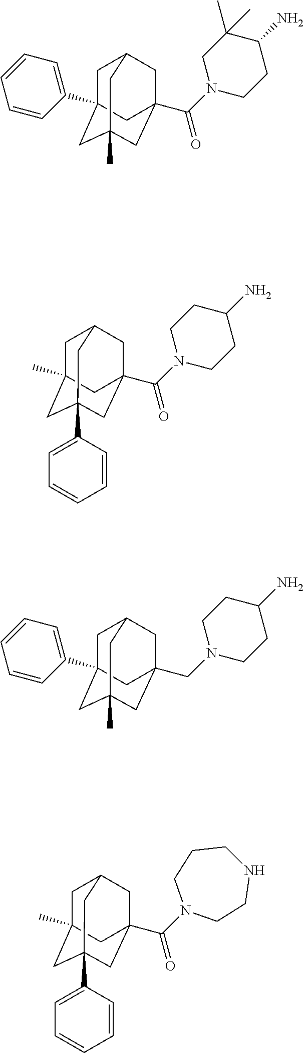 [SMILES: CC1(C)CN(C(=O)C23CC4C[C@](C)(C2)C[C@@](c2ccccc2)(C4)C3)CC[C@H]1N ; C[C@]12CC3CC(C(=O)N4CCC(N)CC4)(C1)C[C@@](c1ccccc1)(C3)C2 ; C[C@@]12CC3CC(CN4CCC(N)CC4)(C1)C[C@](c1ccccc1)(C3)C2 ; C[C@]12CC3CC(C(=O)N4CCCNCC4)(C1)C[C@@](c1ccccc1)(C3)C2]

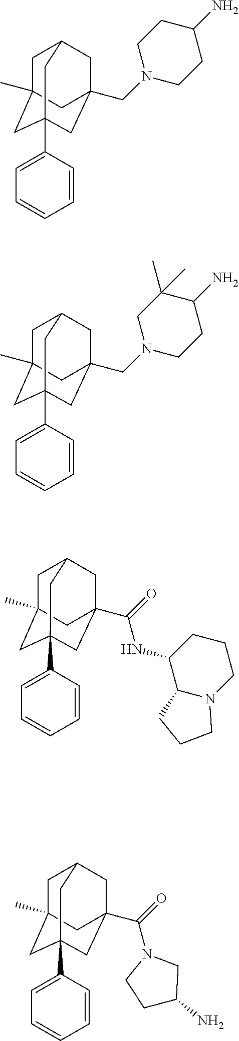 [SMILES: CC12CC3CC(CN4CCC(N)CC4)(C1)CC(c1ccccc1)(C3)C2 ; CC12CC3CC(CN4CCC(N)C(C)(C)C4)(C1)CC(c1ccccc1)(C3)C2 ; C[C@]12CC3CC(C(=O)N[C@@H]4CCCN5CCC[C@H]45)(C1)C[C@@](c1ccccc1)(C3)C2 ; C[C@]12CC3CC(C(=O)N4CC[C@@H](N)C4)(C1)C[C@@](c1ccccc1)(C3)C2]

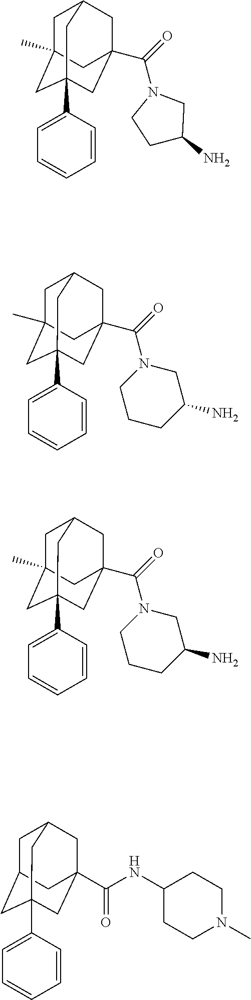 [SMILES: C[C@]12CC3CC(C(=O)N4CC[C@H](N)C4)(C1)C[C@@](c1ccccc1)(C3)C2 ; CC12CC3CC(C(=O)N4CCC[C@@H](N)C4)(C1)C[C@@](c1ccccc1)(C3)C2 ; C[C@]12CC3CC(C(=O)N4CCC[C@H](N)C4)(C1)C[C@@](c1ccccc1)(C3)C2 ; CN1CCC(NC(=O)C23CC4CC(C2)CC(c2ccccc2)(C4)C3)CC1]

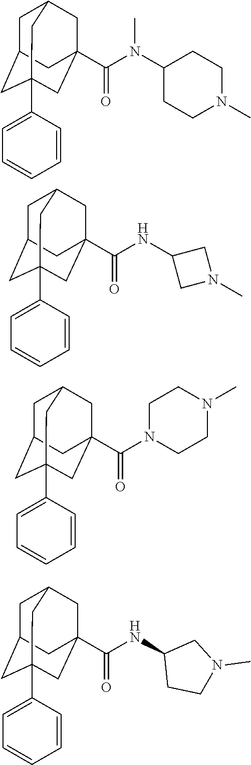

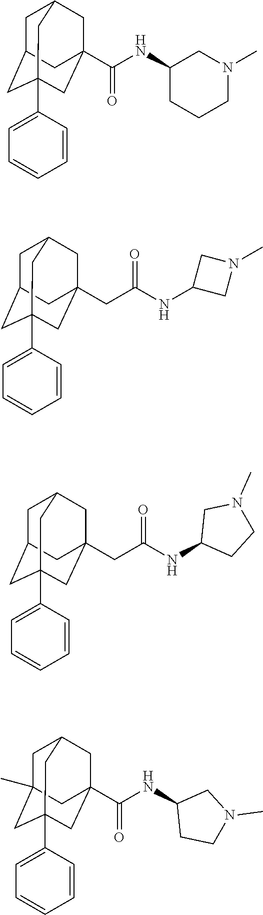

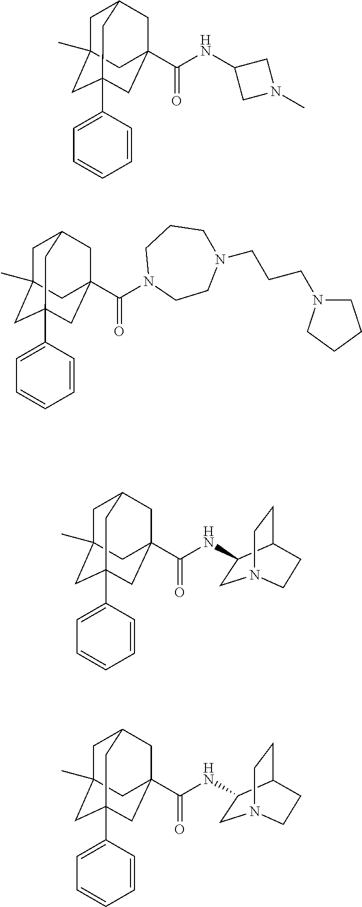

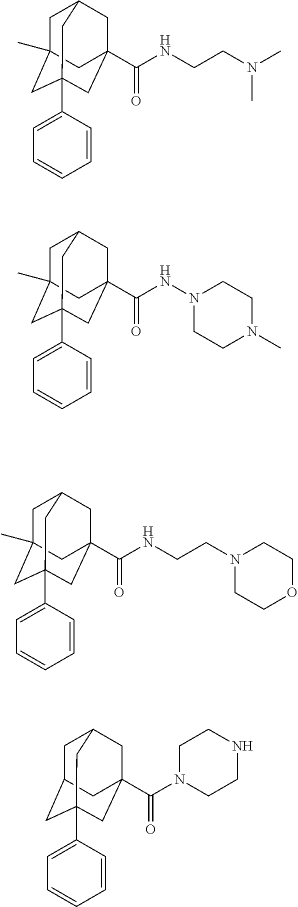

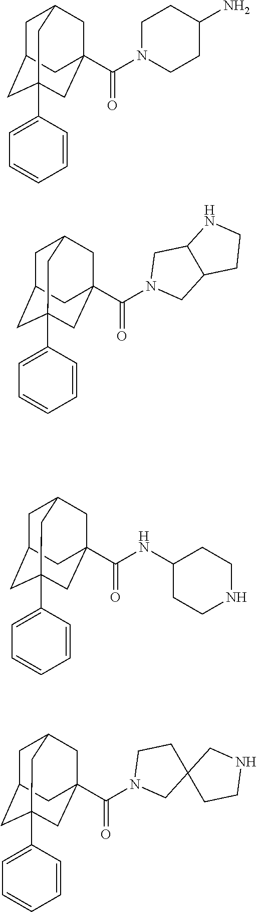 [SMILES: NC1CCN(C(=O)C23CC4CC(C2)CC(c2ccccc2)(C4)C3)CC1 ; O=C(N1CC2CCNC2C1)C12CC3CC(C1)CC(c1ccccc1)(C3)C2 ; O=C(NC1CCNCC1)C12CC3CC(C1)CC(c1ccccc1)(C3)C2 ; O=C(N1CCC2(CCNC2)C1)C12CC3CC(C1)CC(c1ccccc1)(C3)C2]

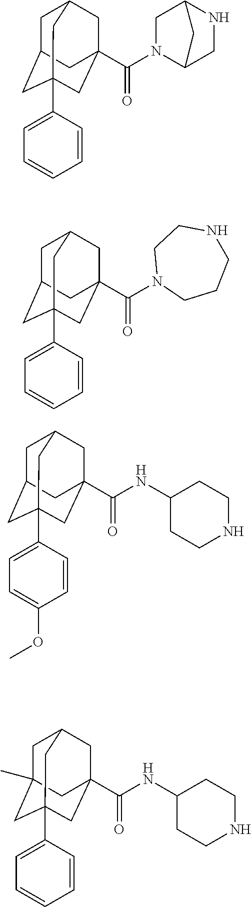

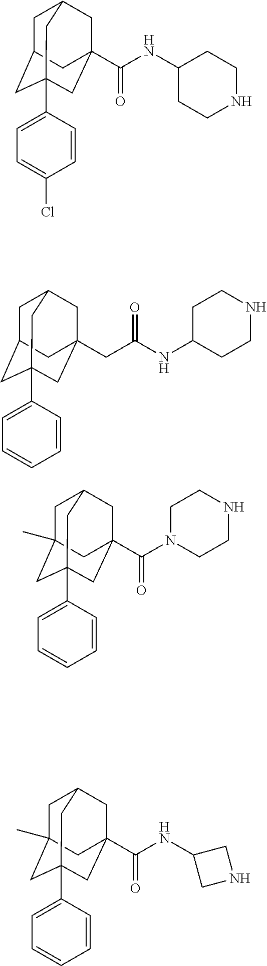

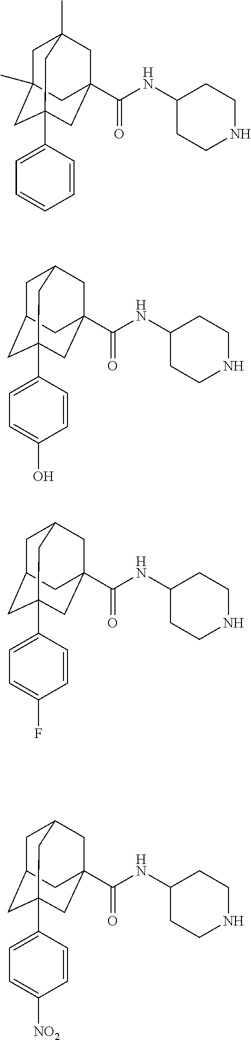 [SMILES: CC12CC3(C)CC(C(=O)NC4CCNCC4)(C1)CC(c1ccccc1)(C2)C3 ; O=C(NC1CCNCC1)C12CC3CC(C1)CC(c1ccc(O)cc1)(C3)C2 ; O=C(NC1CCNCC1)C12CC3CC(C1)CC(c1ccc(F)cc1)(C3)C2 ; O=C(NC1CCNCC1)C12CC3CC(C1)CC(c1ccc([N+](=O)[O-])cc1)(C3)C2]

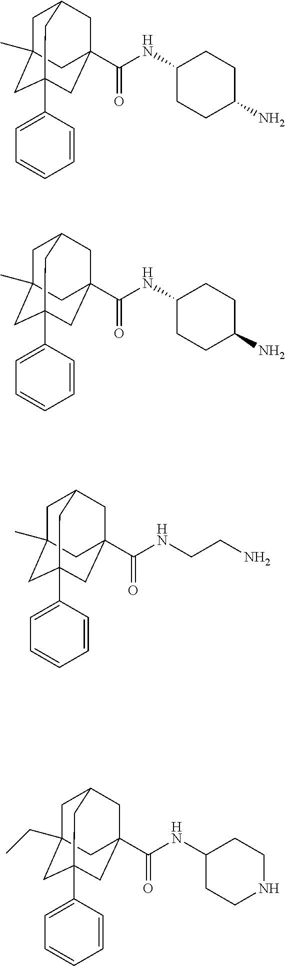 [SMILES: CC12CC3CC(C(=O)N[C@H]4CC[C@@H](N)CC4)(C1)CC(c1ccccc1)(C3)C2 ; CC12CC3CC(C(=O)N[C@H]4CC[C@H](N)CC4)(C1)CC(c1ccccc1)(C3)C2 ; CC12CC3CC(C(=O)NCCN)(C1)CC(c1ccccc1)(C3)C2 ; CCC12CC3CC(C(=O)NC4CCNCC4)(C1)CC(c1ccccc1)(C3)C2]

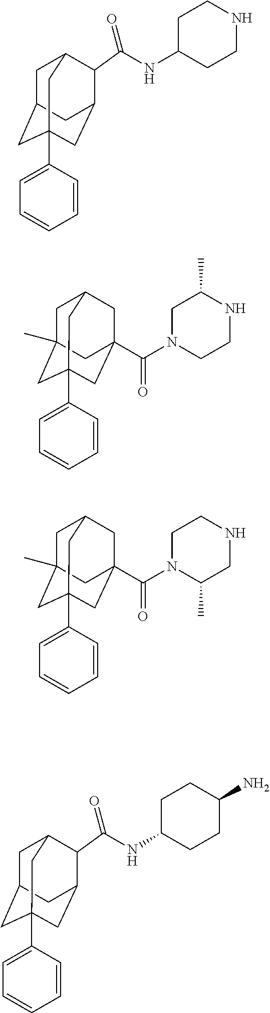 [SMILES: O=C(NC1CCNCC1)C1C2CC3CC1CC(c1ccccc1)(C3)C2 ; C[C@H]1CN(C(=O)C23CC4CC(C)(C2)CC(c2ccccc2)(C4)C3)CCN1 ; C[C@H]1CNCCN1C(=O)C12CC3CC(C)(C1)CC(c1ccccc1)(C3)C2 ; N[C@H]1CC[C@H](NC(=O)C2C3CC4CC2CC(c2ccccc2)(C4)C3)CC1]

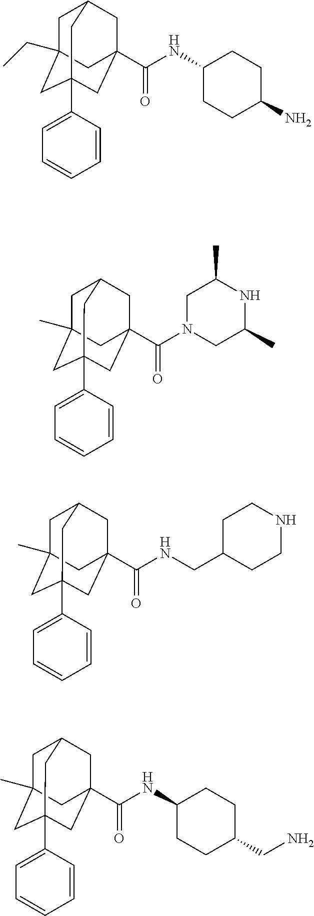

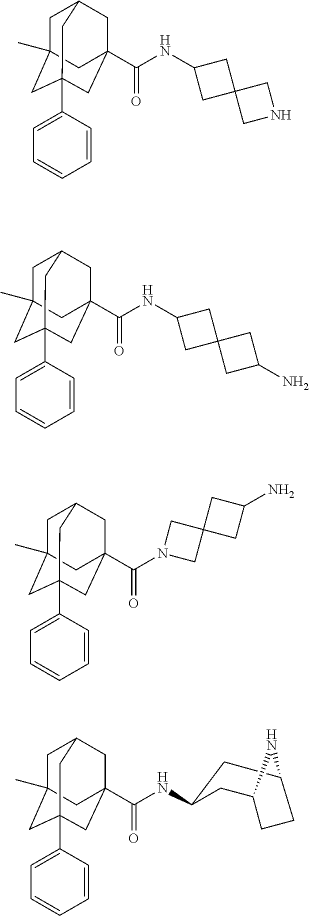

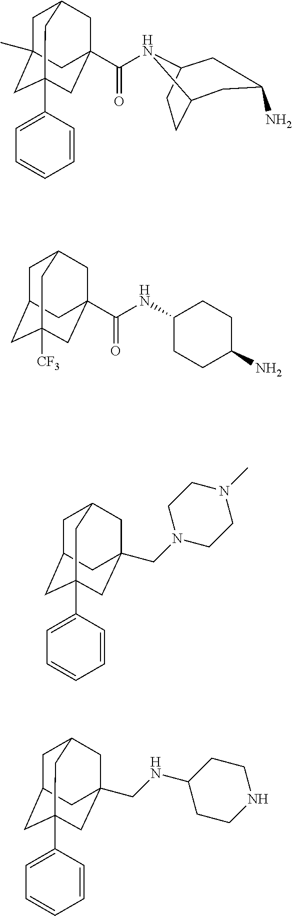

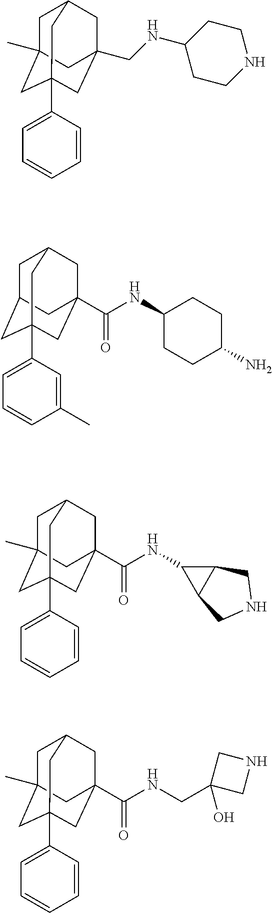

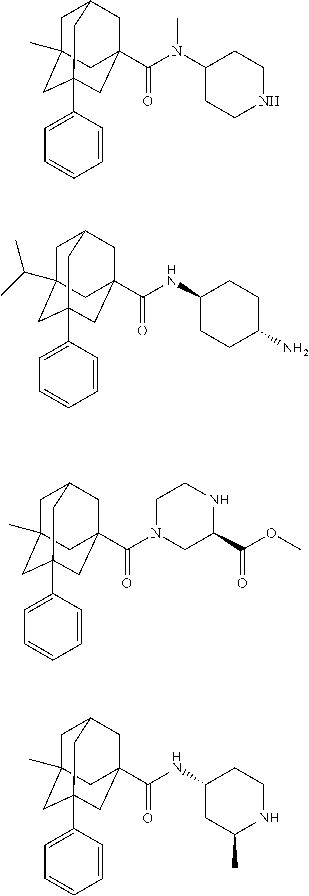

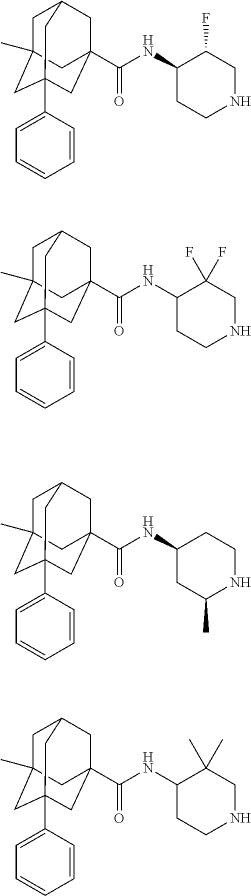 [SMILES: CC12CC3CC(C(=O)N[C@@H]4CCNC[C@H]4F)(C1)CC(c1ccccc1)(C3)C2 ; CC12CC3CC(C(=O)NC4CCNCC4(F)F)(C1)CC(c1ccccc1)(C3)C2 ; C[C@H]1C[C@@H](NC(=O)C23CC4CC(C)(C2)CC(c2ccccc2)(C4)C3)CCN1 ; CC12CC3CC(C(=O)NC4CCNCC4(C)C)(C1)CC(c1ccccc1)(C3)C2]

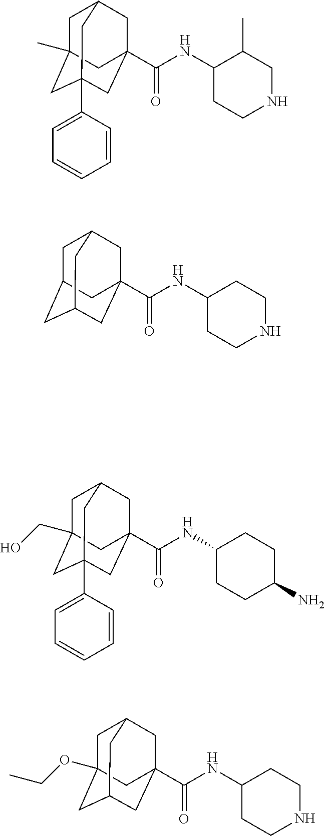 [SMILES: CC1CNCCC1NC(=O)C12CC3CC(C)(C1)CC(c1ccccc1)(C3)C2 ; O=C(NC1CCNCC1)C12CC3CC(CC(C3)C1)C2 ; N[C@H]1CC[C@H](NC(=O)C23CC4CC(CO)(C2)CC(c2ccccc2)(C4)C3)CC1 ; CCOC12CC3CC(C1)CC(C(=O)NC1CCNCC1)(C3)C2]

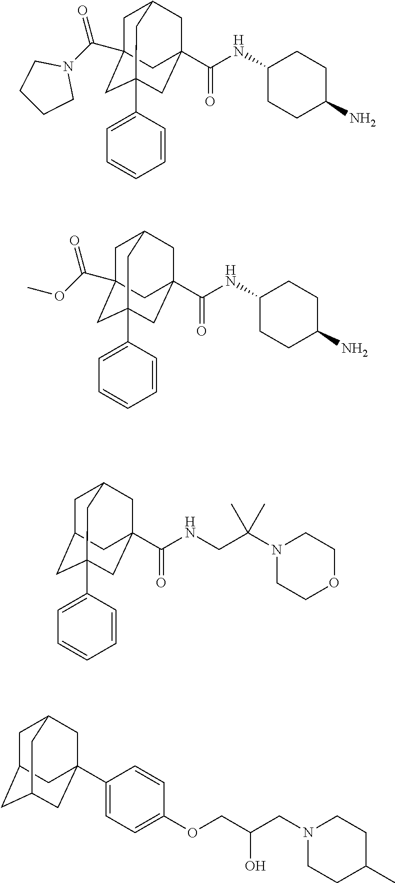 [SMILES: N[C@H]1CC[C@H](NC(=O)C23CC4CC(C(=O)N5CCCC5)(C2)CC(c2ccccc2)(C4)C3)CC1 ; COC(=O)C12CC3CC(C(=O)N[C@H]4CC[C@H](N)CC4)(C1)CC(c1ccccc1)(C3)C2 ; CC(C)(CNC(=O)C12CC3CC(C1)CC(c1ccccc1)(C3)C2)N1CCOCC1 ; CC1CCN(CC(O)COc2ccc(C34CC5CC(CC(C5)C3)C4)cc2)CC1]

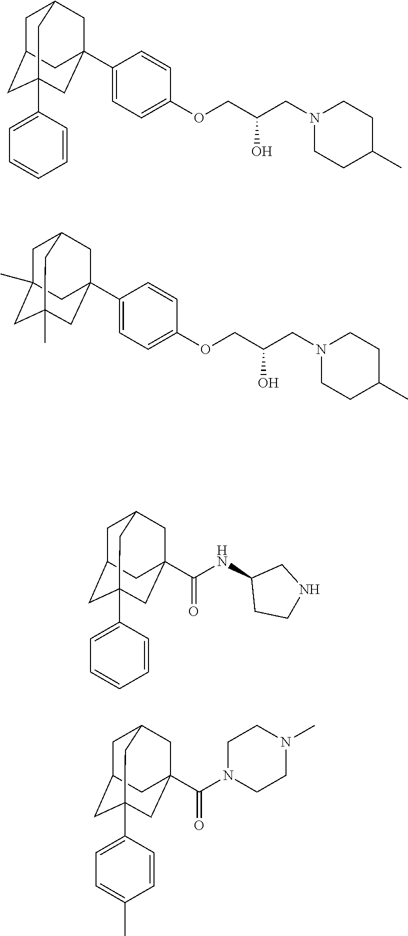

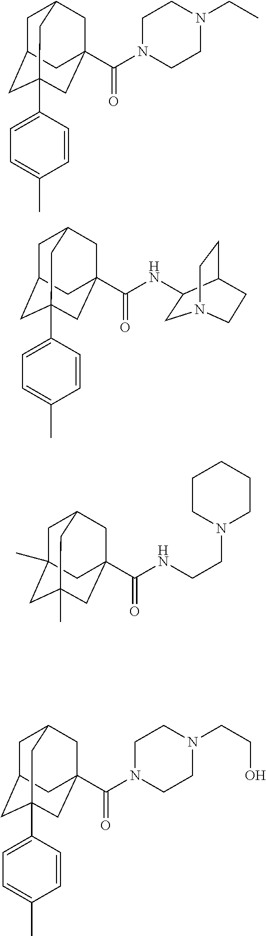

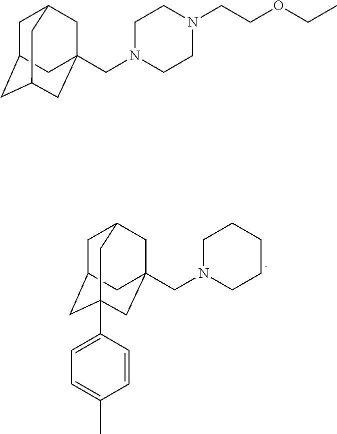 [SMILES: CCOCCN1CCN(CC23CC4CC(CC(C4)C2)C3)CC1 ; Cc1ccc(C23CC4CC(CC(CN5CCCCC5)(C4)C2)C3)cc1]

2. The method of claim 1, wherein X is 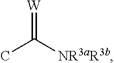
Y is CH₂, W is selected from O and S, and Q is CH₂.

3. The method of claim 2, wherein:
W is O;
R¹ is phenyl;
NR^{3a}R^{3b} is selected from the group consisting of

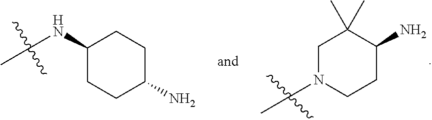

4. The method of claim 2, wherein:
W is S;
R¹ is phenyl;
R² is selected from the group consisting of halogen, methyl, ethyl, propyl, chloromethyl, chloroethyl, methylthiomethyl, and thiomethyl; and wherein
NR^{3a}R^{3b} is selected from the group consisting of

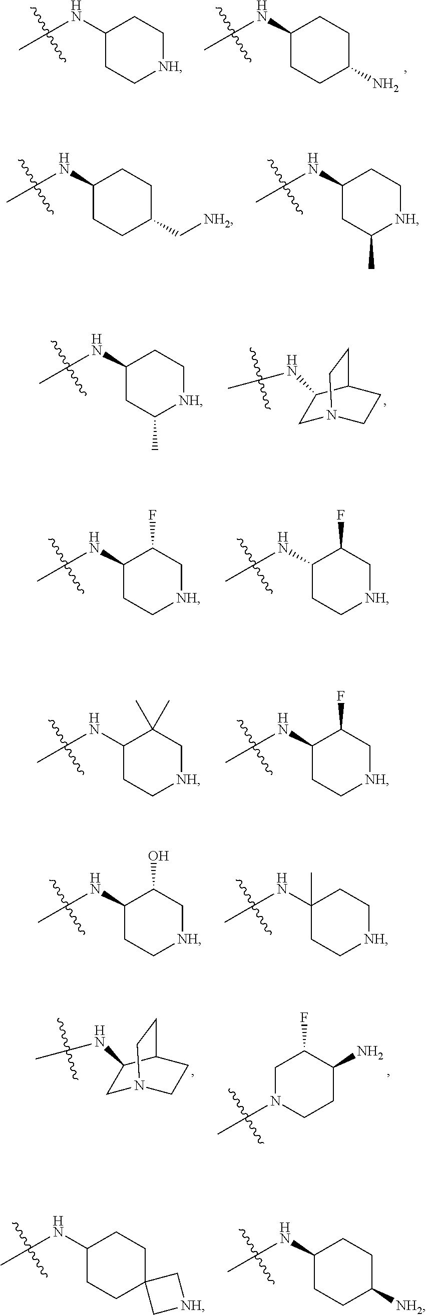

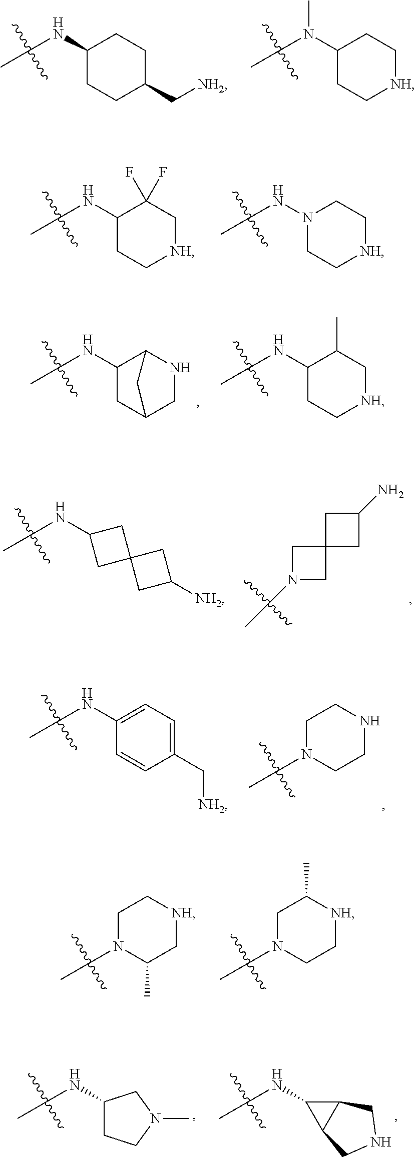

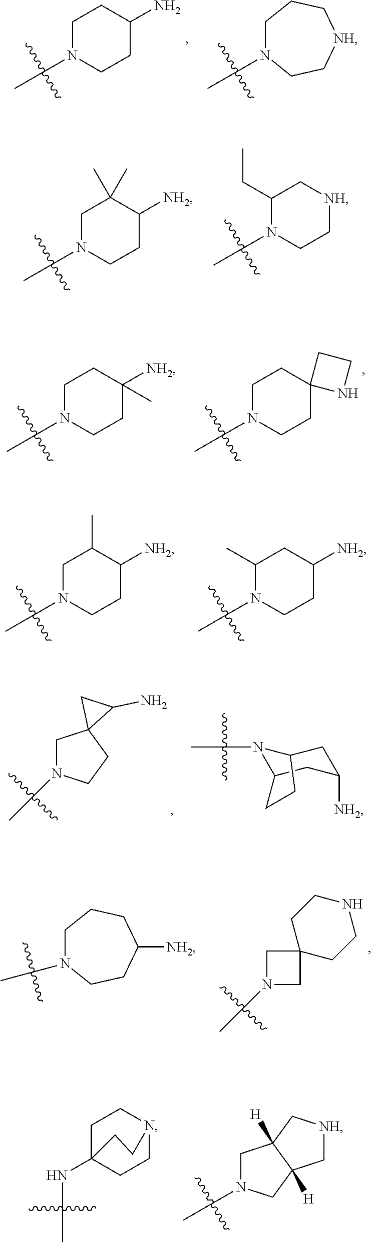

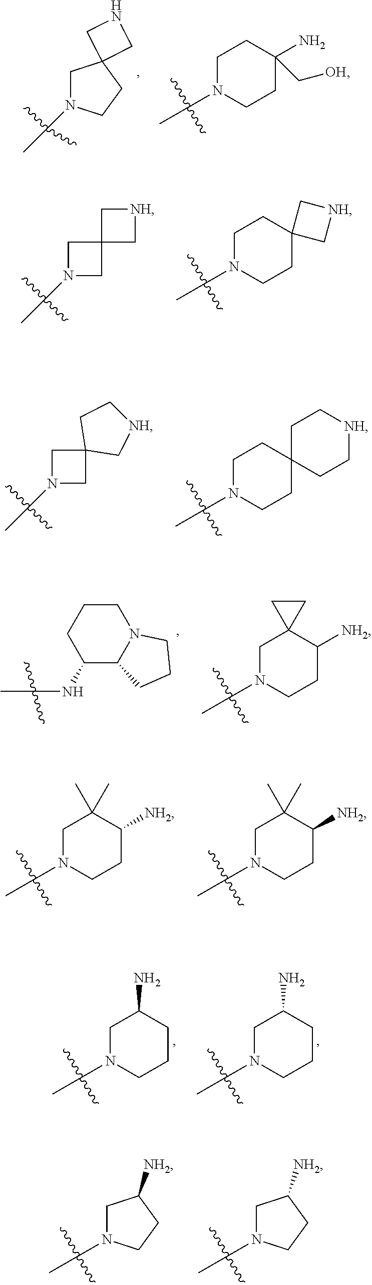

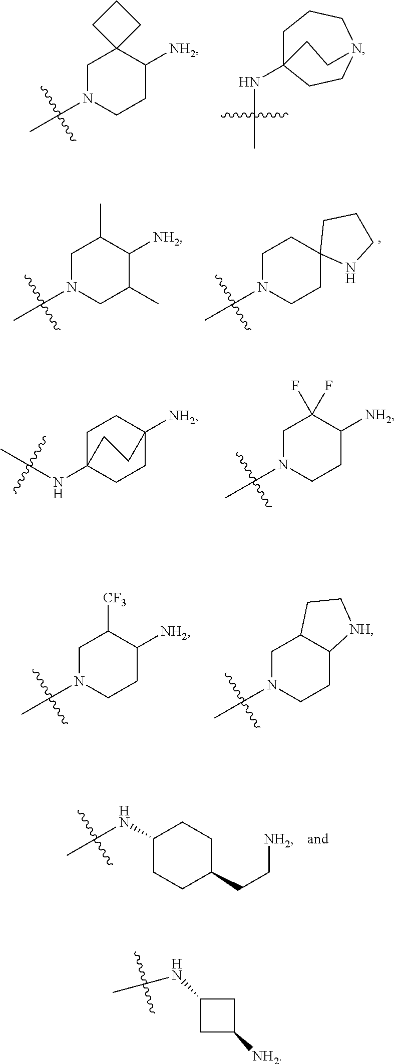

5. The method of claim 5, wherein X is 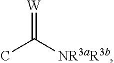
Y is CH₁, W is selected from O and S, and Q is CR²³R²⁴.

6. The method of claim 5, wherein:
R¹ is phenyl;
NR^{3a}R^{3b} is selected from the group consisting of 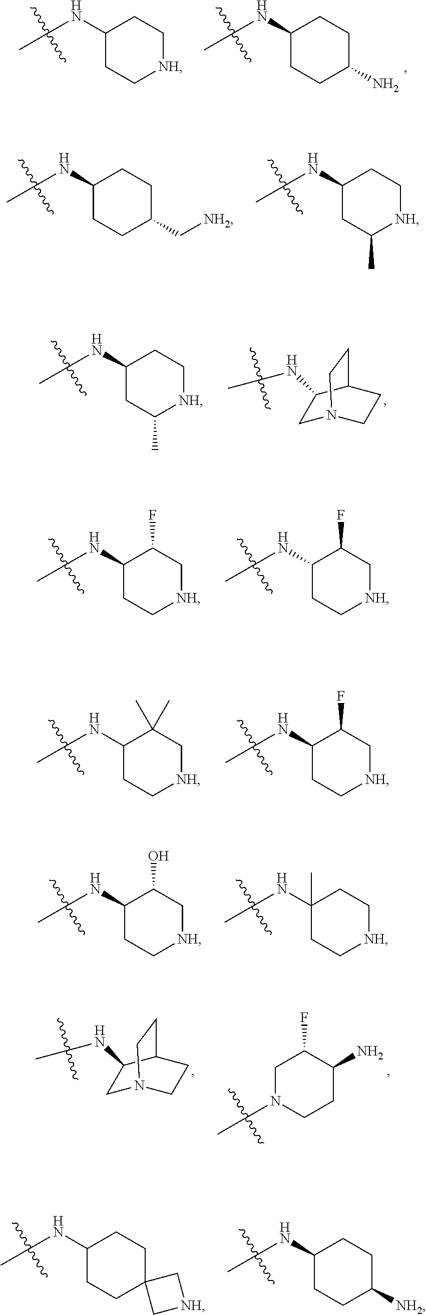 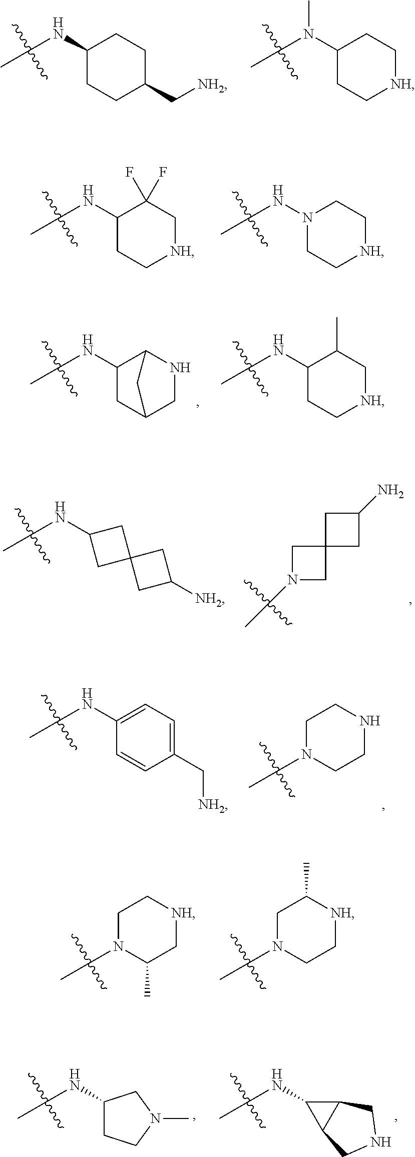 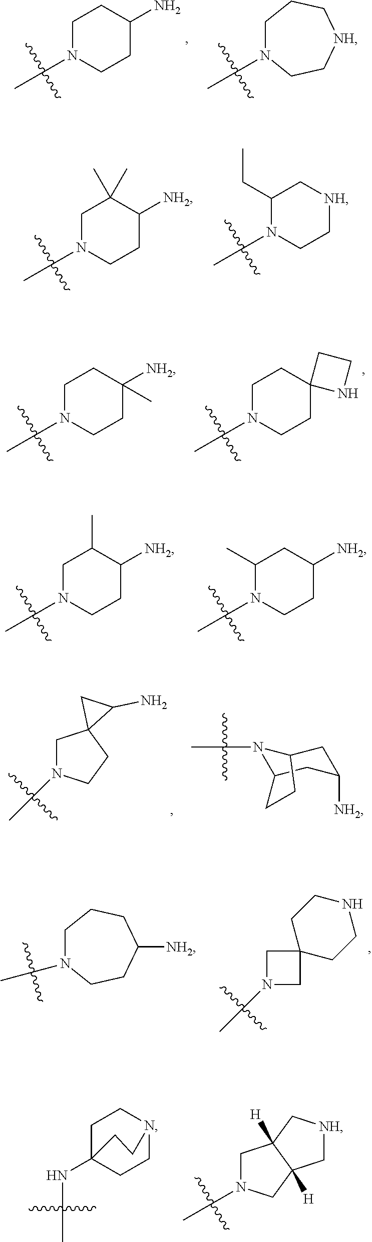 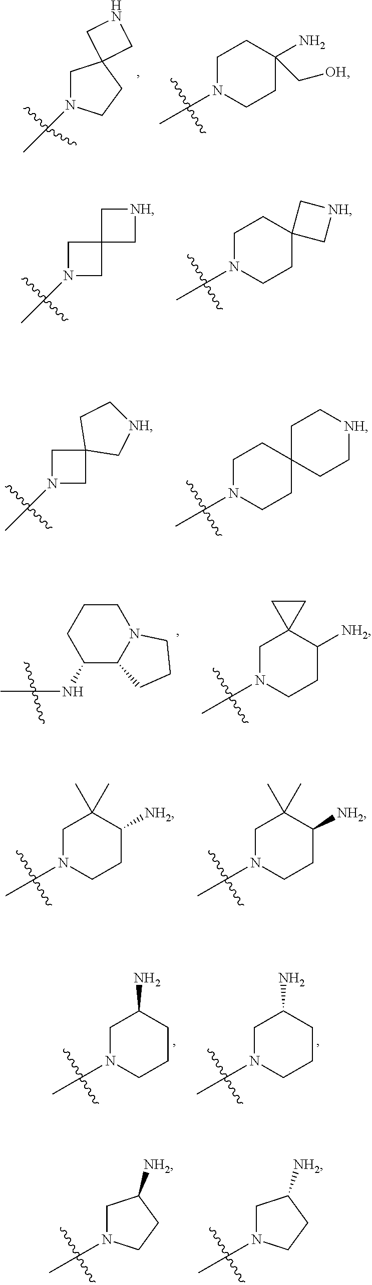 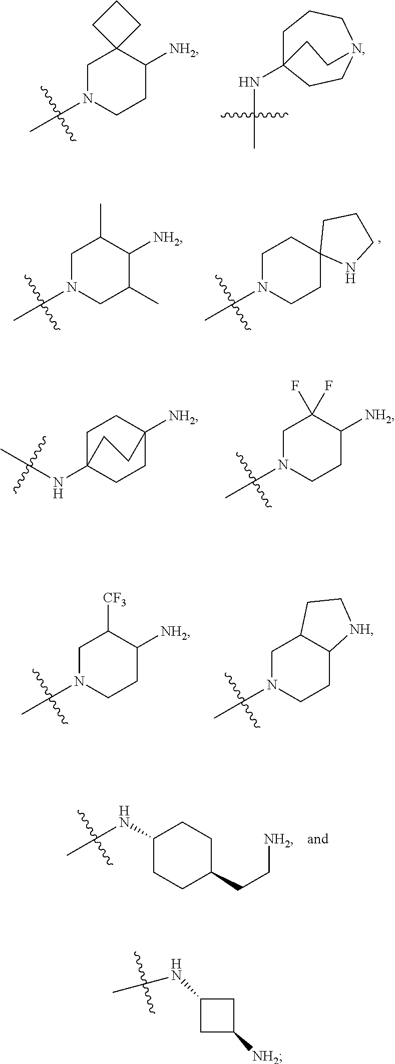
and
wherein
R²³ is selected from the group consisting of hydrogen, halogen, methyl, ethyl, propyl, butyl, benzyl, phenethyl, methoxy, ethoxy, phenoxy, benzyloxy, chloromethyl, fluoromethyl, difluoromethyl, methylthiomethyl, etylthiomethyl, 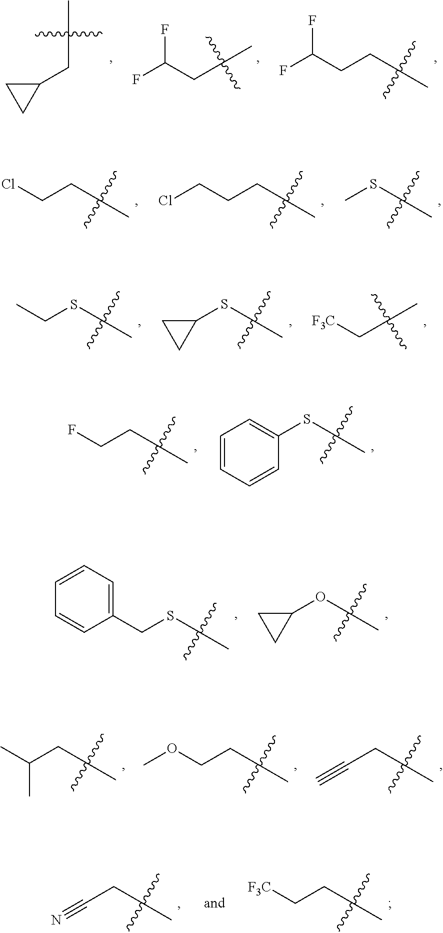
and R²⁴ is selected from the group consisting of halogen, methyl, ethyl, propyl, butyl, benzyl, phenethyl, methoxy, ethoxy, phenoxy, benzyloxy, chloromethyl, fluoromethyl, difluoromethyl, methylthiomethyl, etylthiomethyl, 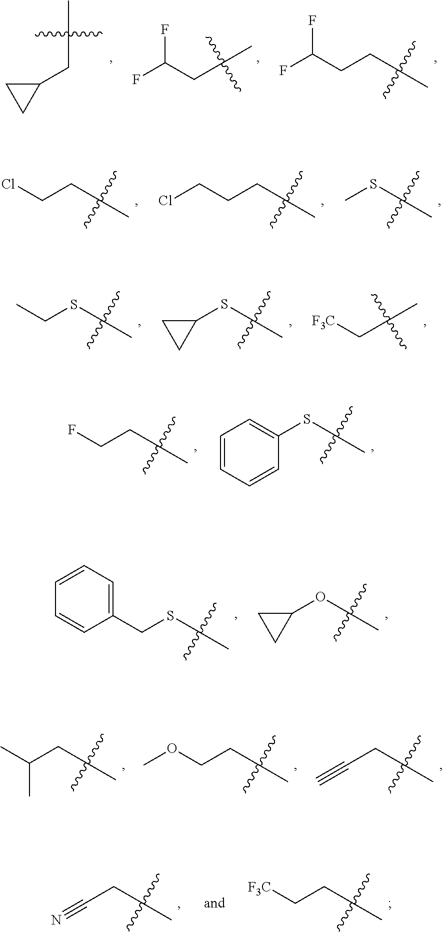
or R²³ and R²⁴ may be taken together with the carbon atom to which they are attached to form a cyclopropyl, cyclobutyl, or cyclopentyl;
or R²³ and R²⁴ may be taken together with the carbon atom to which they are attached to form a carbonyl,

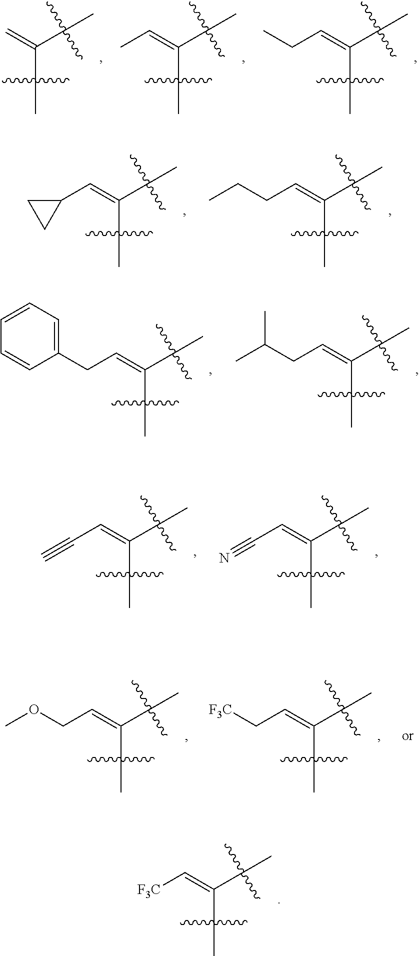

7. The method of claim 1, wherein X is CH, Y is 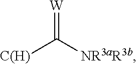
W is selected from O and S, and Q is CH₂.

8. The method of claim 7, wherein:
W is S or O;
R¹ is phenyl;
R² is selected from the group consisting of halogen, methyl, ethyl, propyl, chloromethyl, chloroethyl, methylthiomethyl, and thiomethyl; and wherein
NR^{3a}R^{3b} is selected from the group consisting of

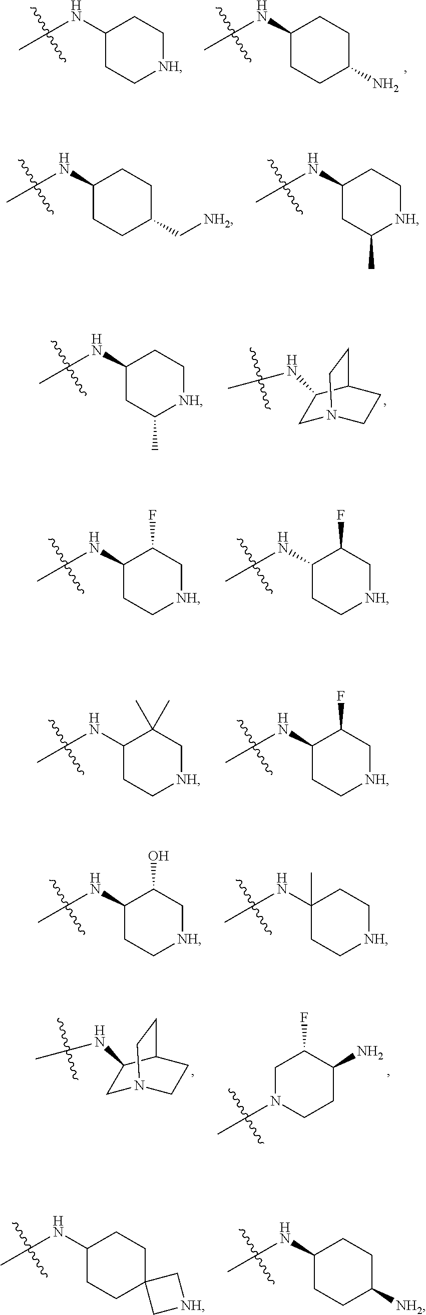

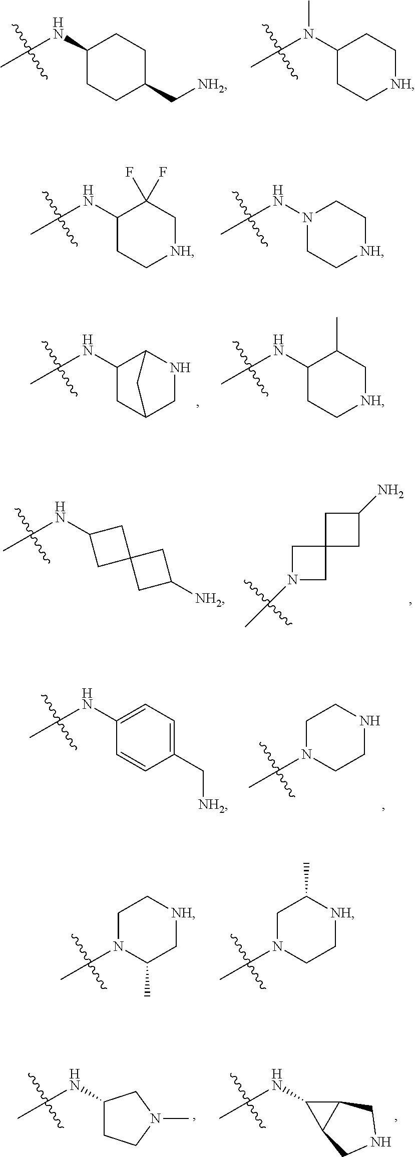

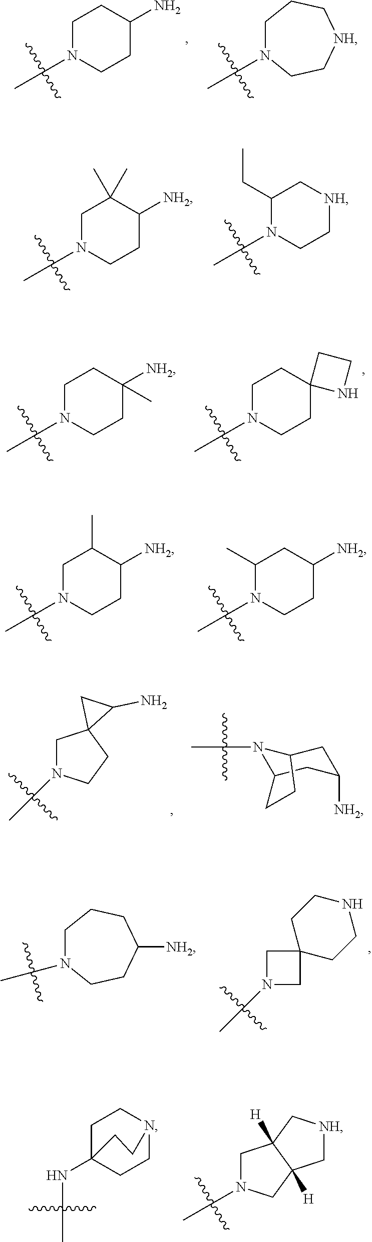

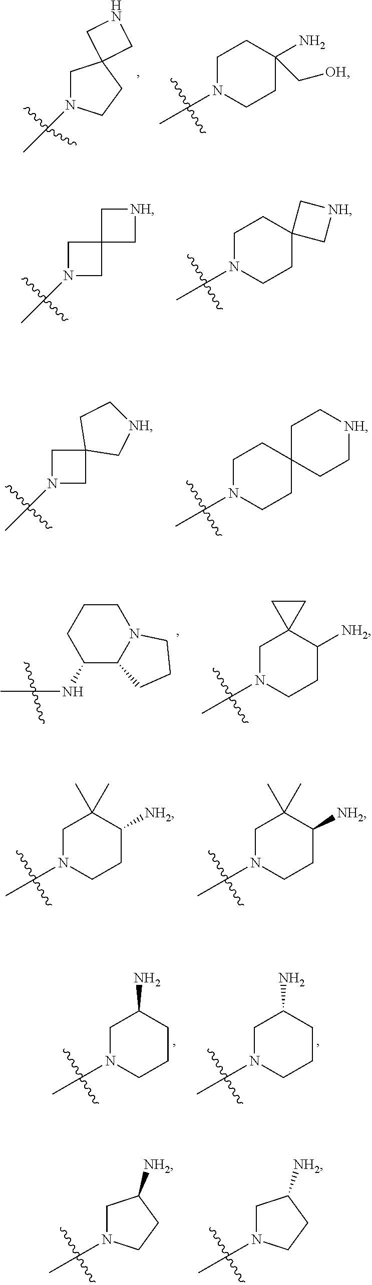

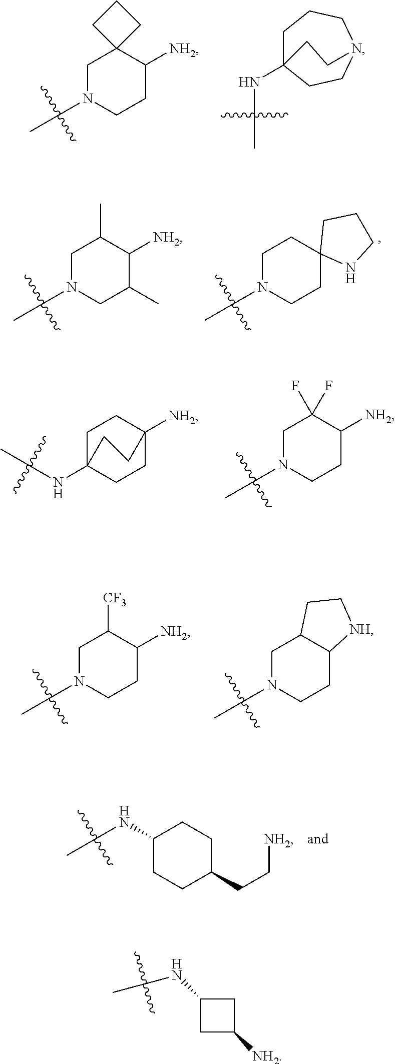

9. The method of claim 1, including administering a therapeutic amount of a therapeutic agent selected from the group consisting of Ribavirin, viral RNA-dependent-RNA polymerase inhibitors including favipiravir, Triazavirin, Remdesivir (GS-5734), monoclonal antibody therapies including, ZMapp, REGN3470-3471-3479, mAb 114, vaccines including, cAd3-EBOZ, rVSV-ZEBOV, small interfering RNAs and microRNAs and immunomodulators.

10. The method of claim 1, wherein Structural Formula I is represented by the following enantiomerically pure compounds of Structural Formulae II and III 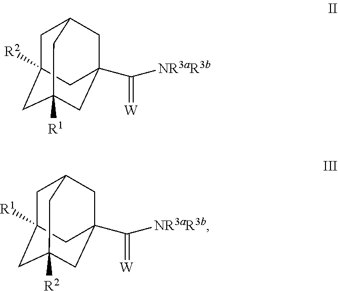
or a pharmaceutically acceptable salt, and a pharmaceutically acceptable carrier, diluent, or vehicle thereof.

11. The method of claim 10, wherein:
W is O;
R¹ is phenyl;
NR^{3a}R^{3b} is selected from the group consisting of

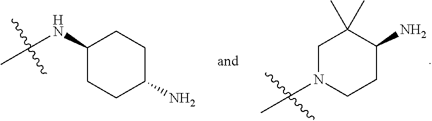

12. The method of claim 10, wherein:
W is S;
R¹ is phenyl;
R² is selected from the group consisting of halogen, methyl, ethyl, propyl, chloromethyl, chloroethyl, methylthiomethyl, and thiomethyl; and wherein
NR^{3a}R^{3b} is selected from the group consisting of

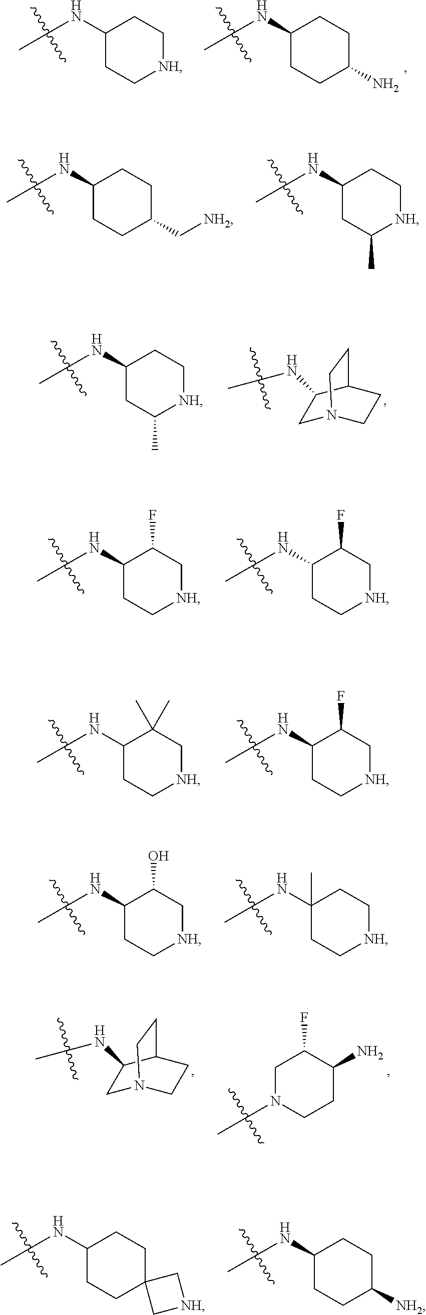

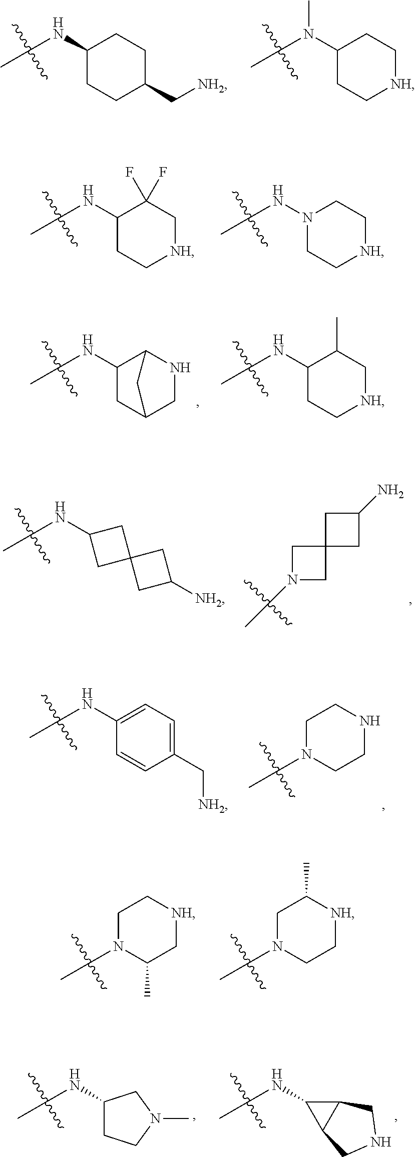

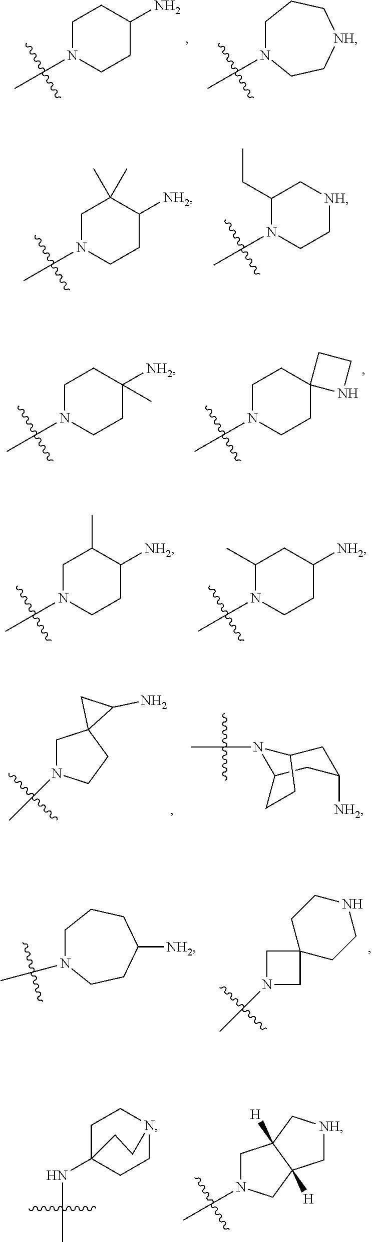

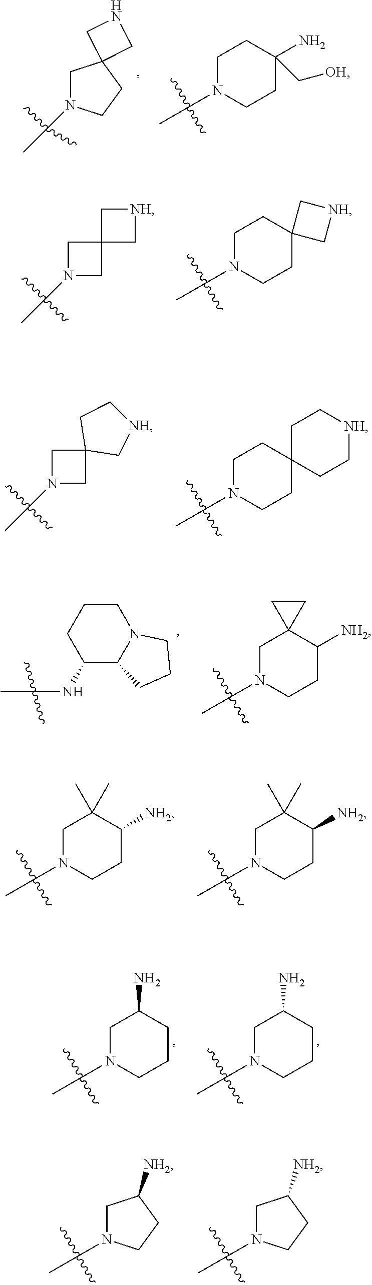

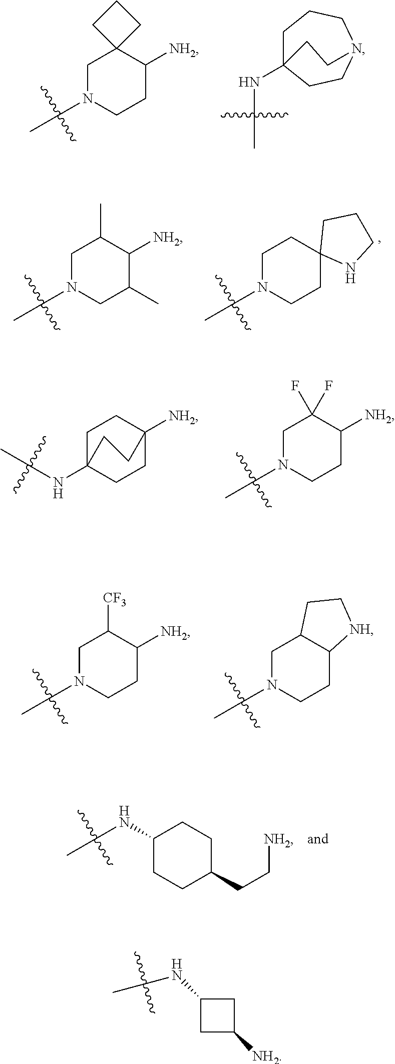

13. The method of claim 10, including administering a therapeutic amount of a therapeutic agent selected from the group consisting of Ribavirin, viral RNA-dependent-RNA polymerase inhibitors including favipiravir, Triazavirin, Remdesivir (GS-5734), monoclonal antibody therapies including, ZMapp, REGN3470-3471-3479, mAb 114, vaccines including, cAd3-EBOZ, rVSV-ZEBOV, small interfering RNAs and microRNAs and immunomodulators.

14. The method of claim 1, wherein Structural Formula I is represented by the following compounds of Structural Formulae IV, V, and VI 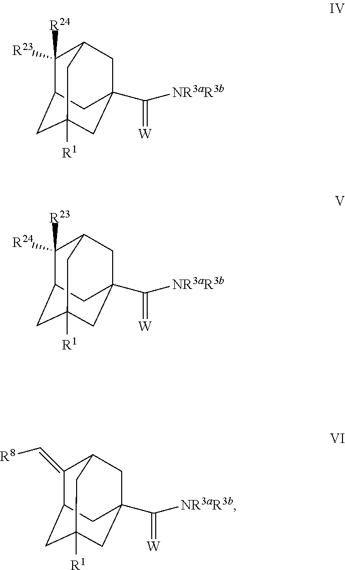
or a pharmaceutically acceptable salt, and a pharmaceutically acceptable carrier, diluent, or vehicle thereof.

15. The method of claim 14, wherein:
W is O or S;
R¹ is phenyl;
NR^{3a}R^{3b} is selected from the group consisting of 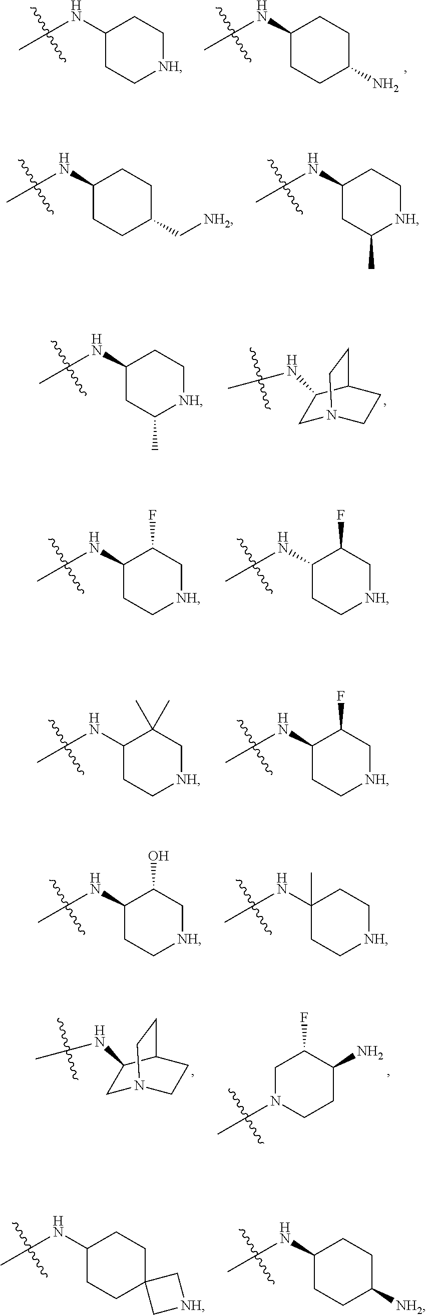 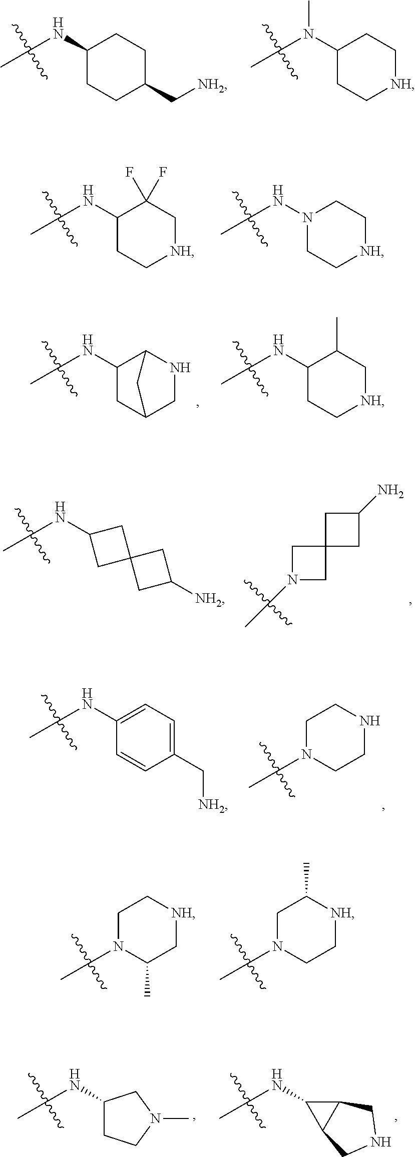 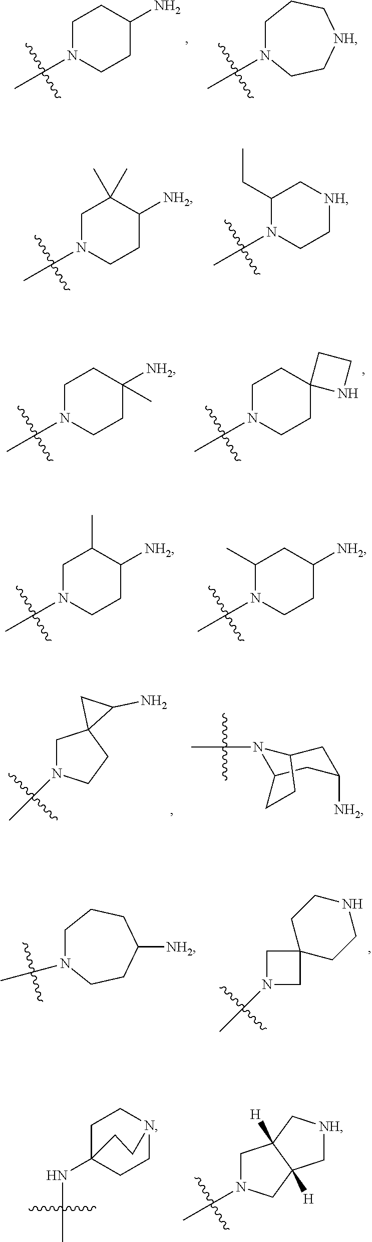 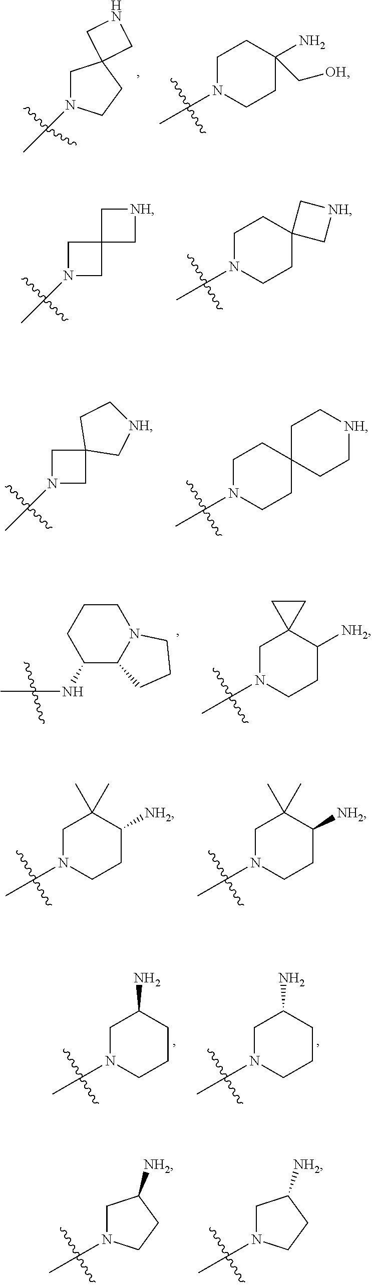 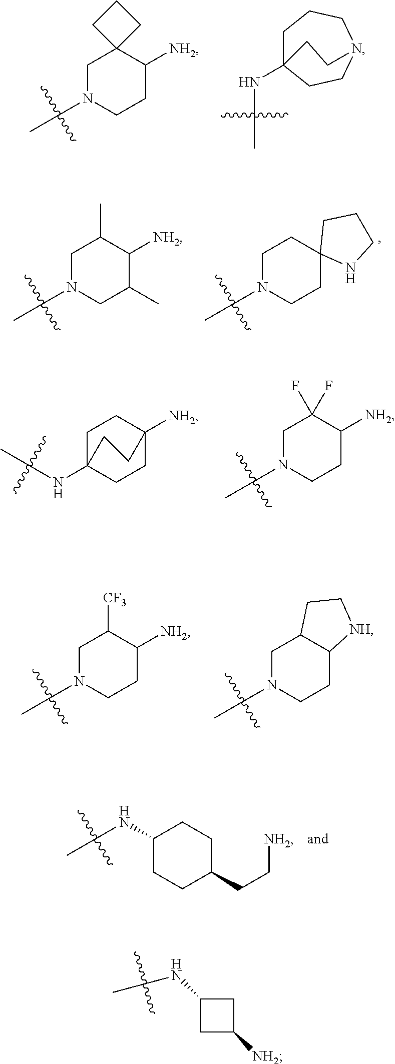
and
wherein
R²³ is selected from the group consisting of hydrogen, halogen, methyl, ethyl, propyl, butyl, benzyl, phenethyl, methoxy, ethoxy, phenoxy, benzyloxy, chloromethyl, fluoromethyl, difluoromethyl, methylthiomethyl, etylthiomethyl, 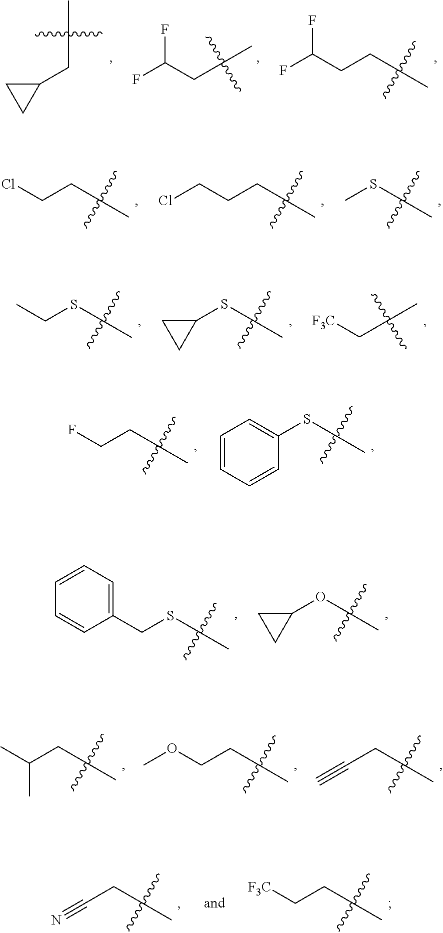
and R²⁴ is selected from the group consisting of halogen, methyl, ethyl, propyl, butyl, benzyl, phenethyl, methoxy, ethoxy, phenoxy, benzyloxy, chloromethyl, fluoromethyl, difluoromethyl, methylthiomethyl, etylthiomethyl, 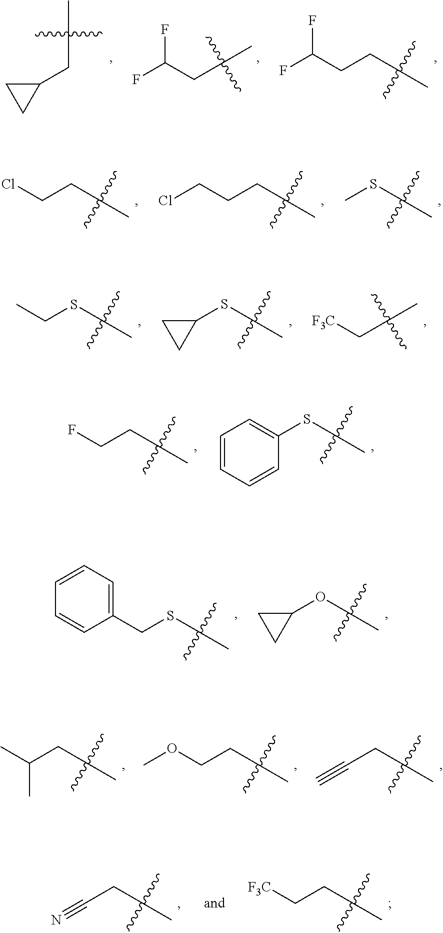
or R²³ and R²⁴ may be taken together with the carbon atom to which they are attached to cyclopropyl, cyclobutyl, or cyclopentyl;
and wherein R⁸ is selected from the group consisting of hydrogen, methyl, ethyl, propyl, cyclopropyl, benzyl, cyano, ethynyl, methoxymethyl, trifluoromethyl, trifluoroethyl, and

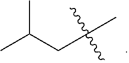 [SMILES: *CC(C)C]

16. The method of claim 14, wherein Structural Formula VI is represented by the following enantiomerically pure compounds of Structural Formulae VIa and VIb 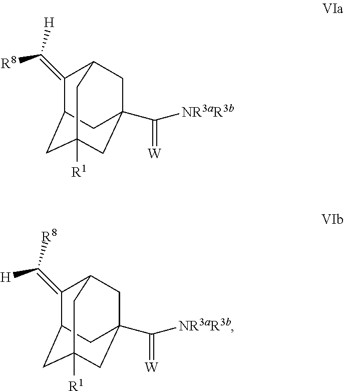
or a pharmaceutically acceptable salt, and a pharmaceutically acceptable carrier, diluent, or vehicle thereof, wherein:
R⁸ is selected from the group consisting of methyl, ethyl, propyl, cyclopropyl, benzyl, cyano, ethynyl, methoxymethyl, trifluoromethyl, trifluoroethyl, and

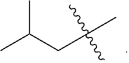 [SMILES: *CC(C)C]

17. The method of claim 14, including administering a therapeutic amount of a therapeutic agent selected from the group consisting of Ribavirin, viral RNA-dependent-RNA polymerase inhibitors including favipiravir, Triazavirin, Remdesivir (GS-5734), monoclonal antibody therapies including, ZMapp, REGN3470-3471-3479, mAb 114, vaccines including, cAd3-EBOZ, rVSV-ZEBOV, small interfering RNAs and microRNAs and immunomodulators.

18. The method of claim 1, wherein Structural Formula I is represented by the following compounds of Structural Formulae VII and VIII 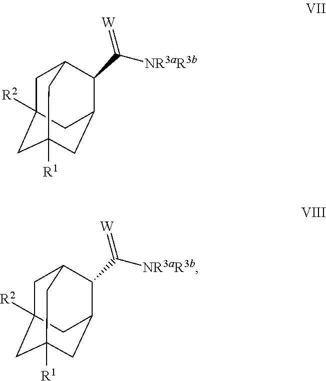
or a pharmaceutically acceptable salt, and a pharmaceutically acceptable carrier, diluent, or vehicle thereof.

19. The method of claim 18, wherein:
W is O or S;
R¹ is phenyl; and wherein
NR^{3a}R^{3b} is selected from the group consisting of

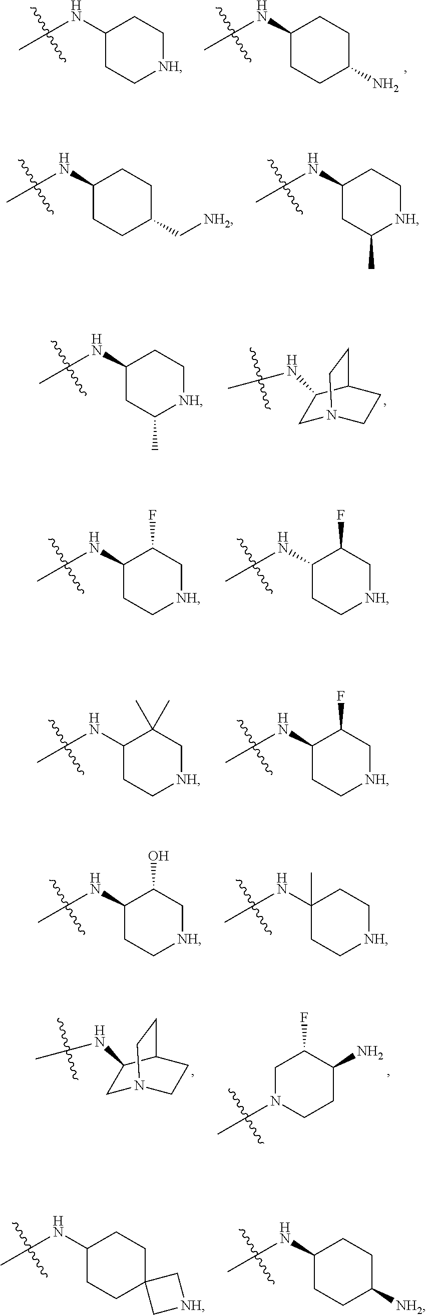

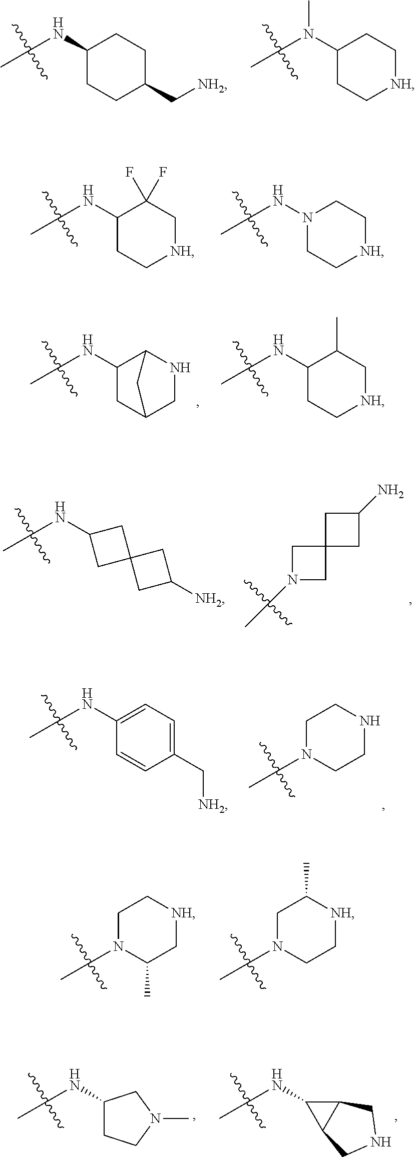

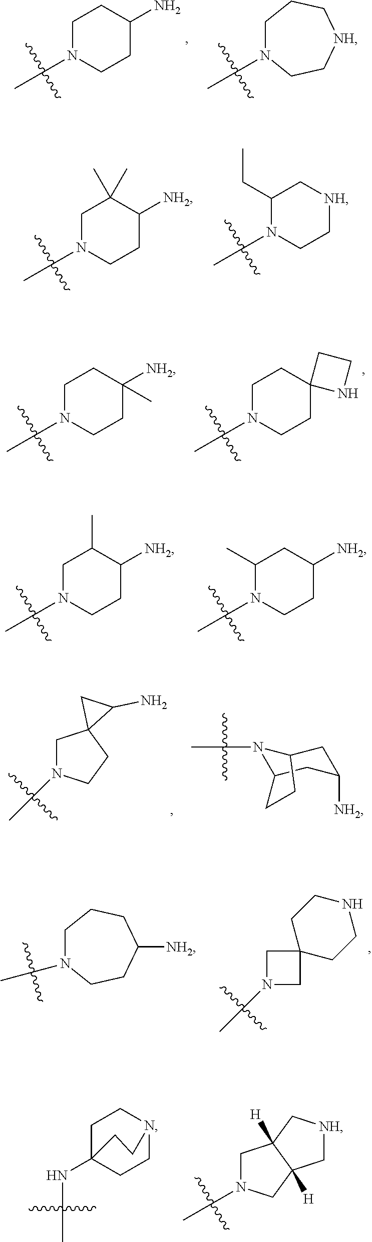

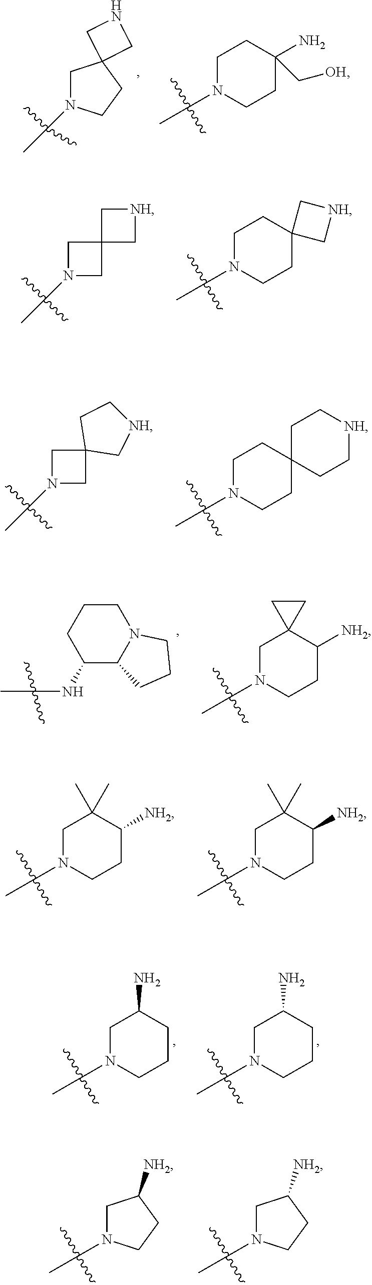

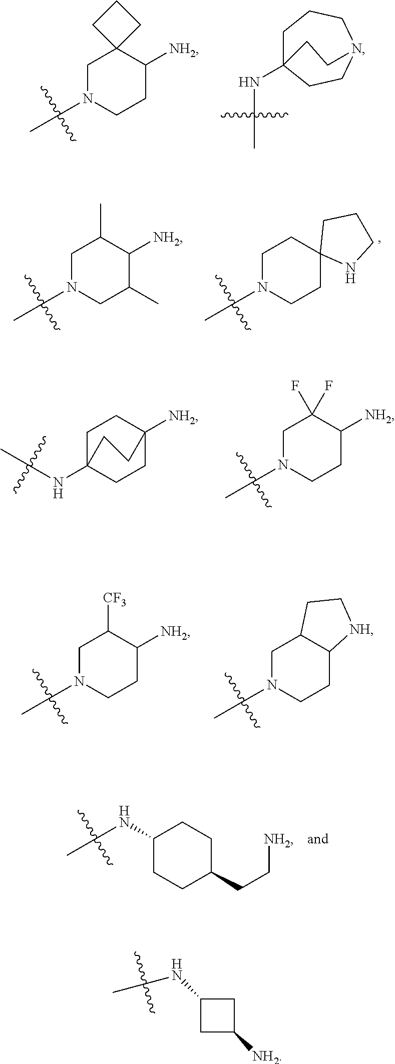

20. A method of treating infections associated with viruses of the Ebolavirus enveloped virus, or any virus expressing filovirus glycoproteins to mediate cell entry comprising administration of a therapeutically effective amount of a compound or a pharmaceutically acceptable salt, and a pharmaceutically acceptable carrier, diluent, or vehicle thereof, selected from the group consisting of:

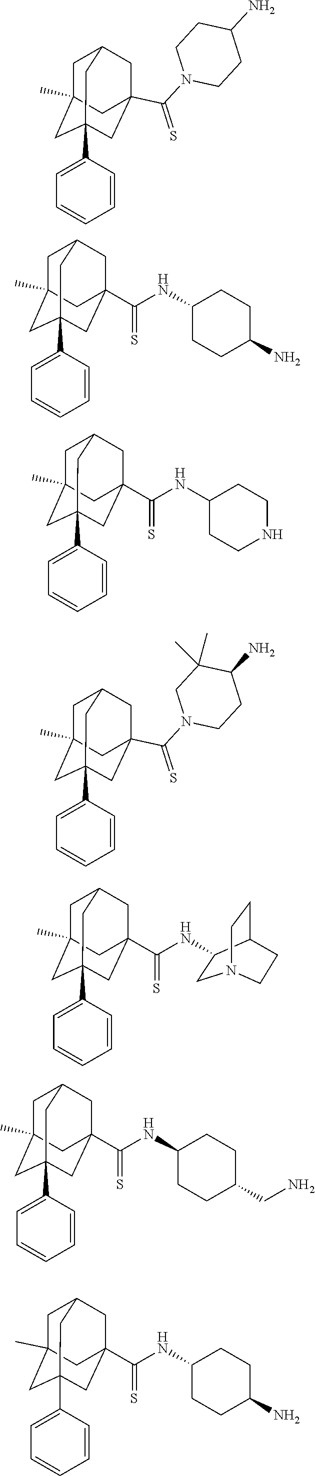

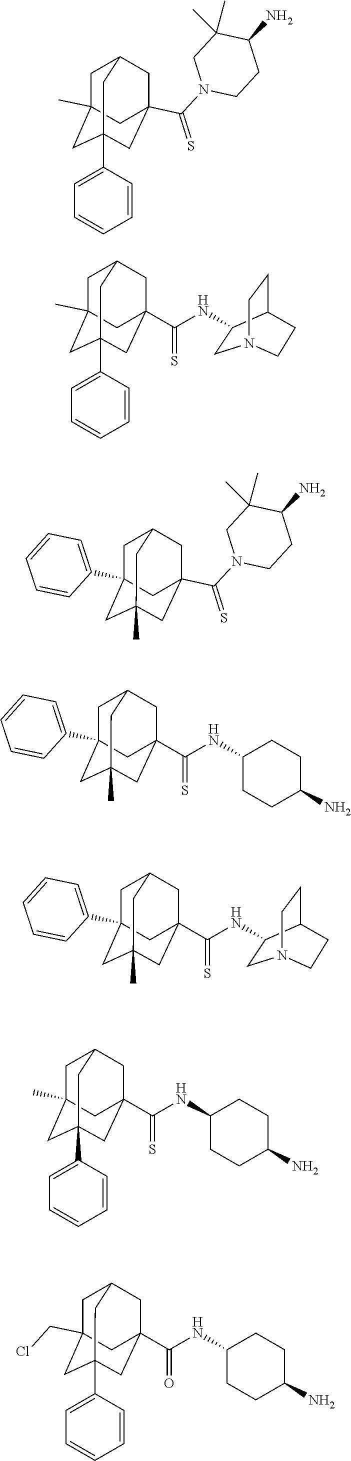

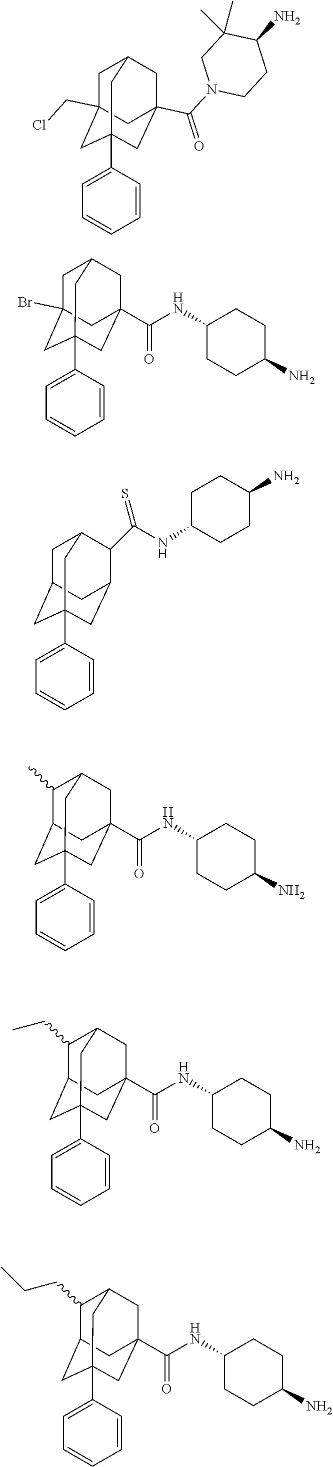

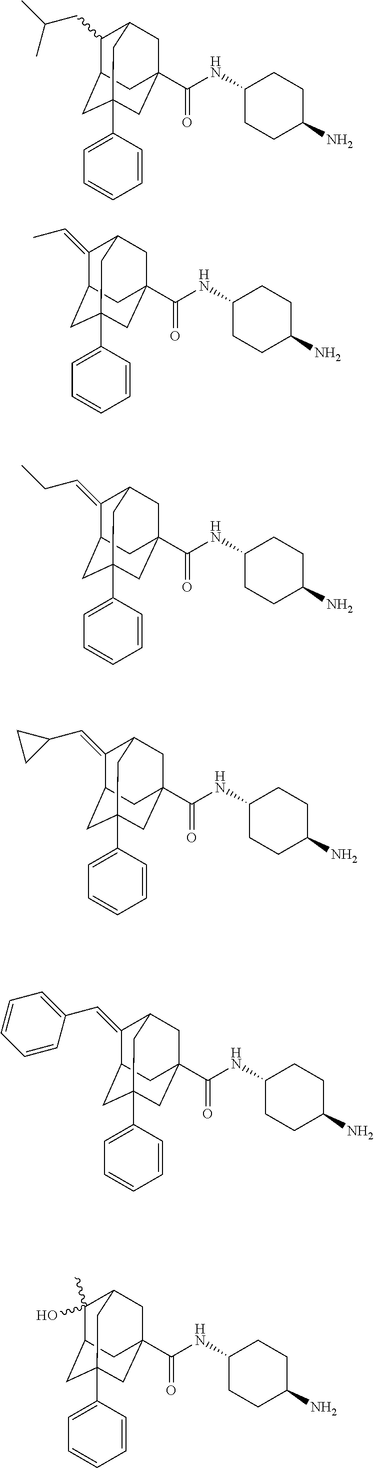

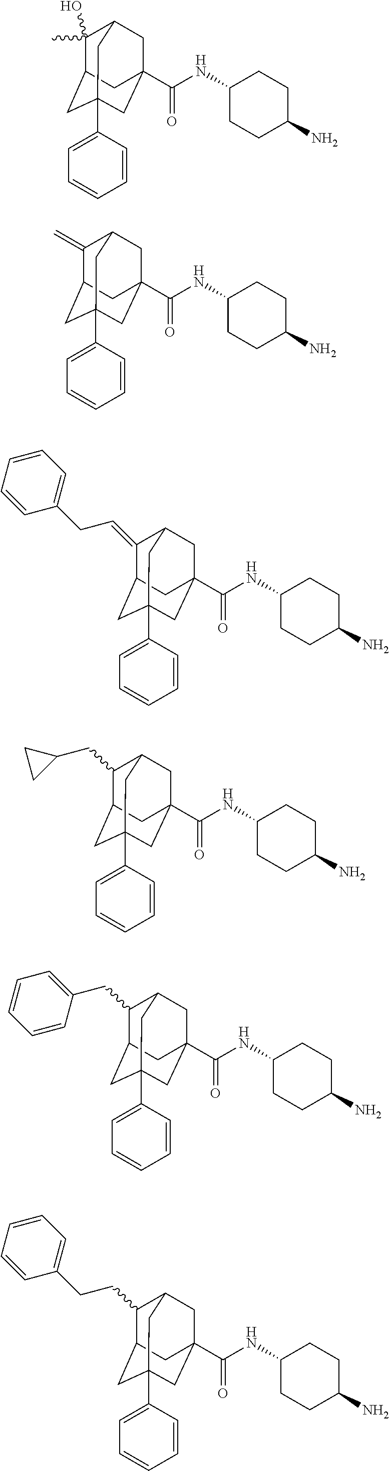

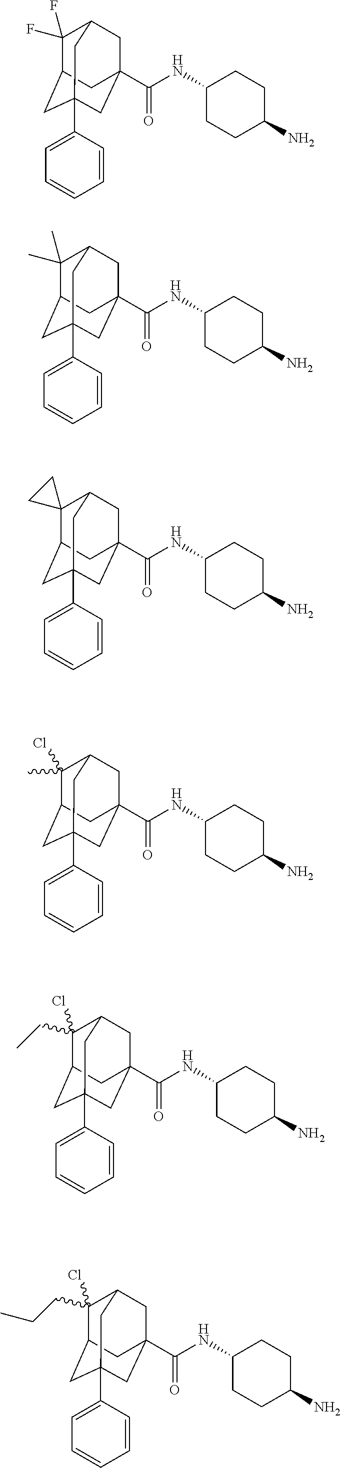

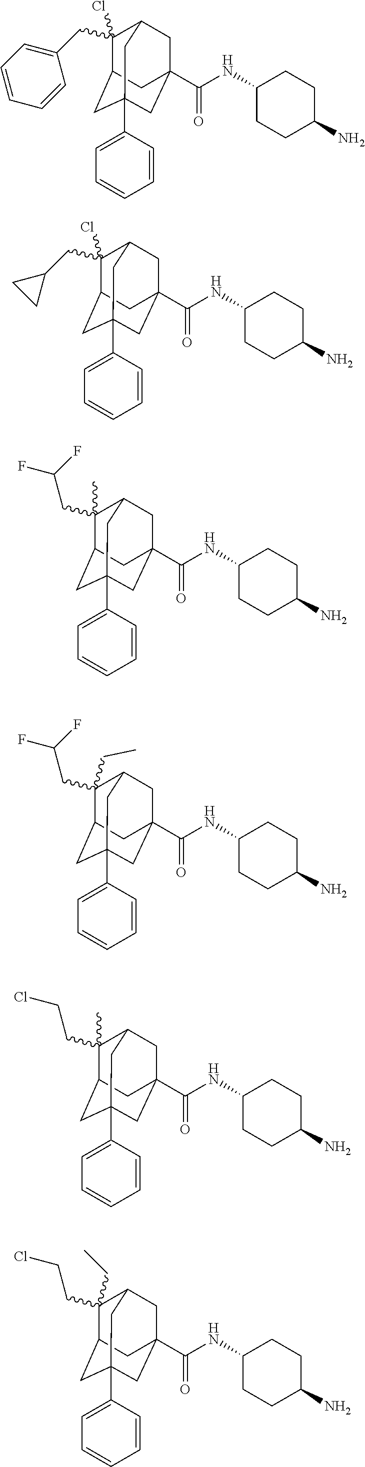

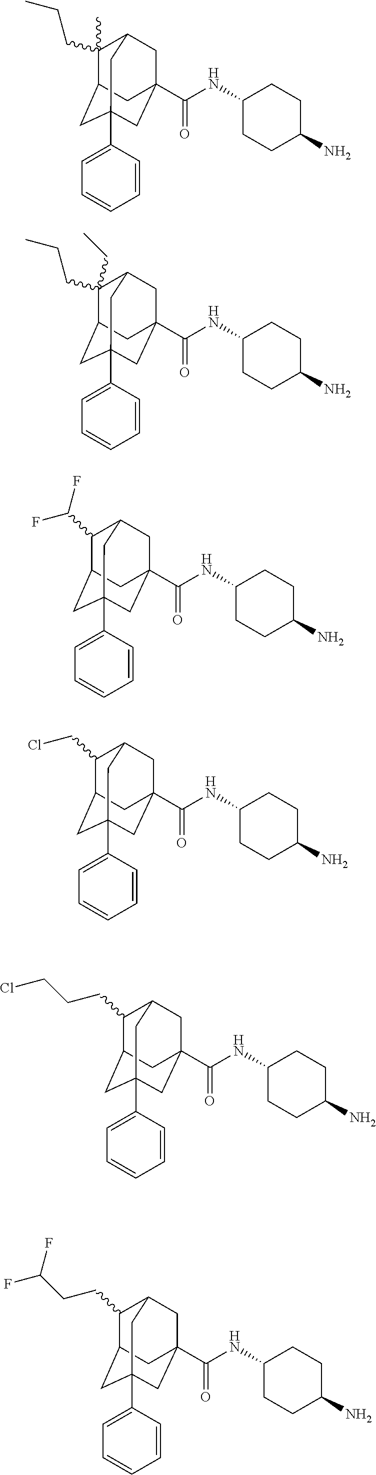

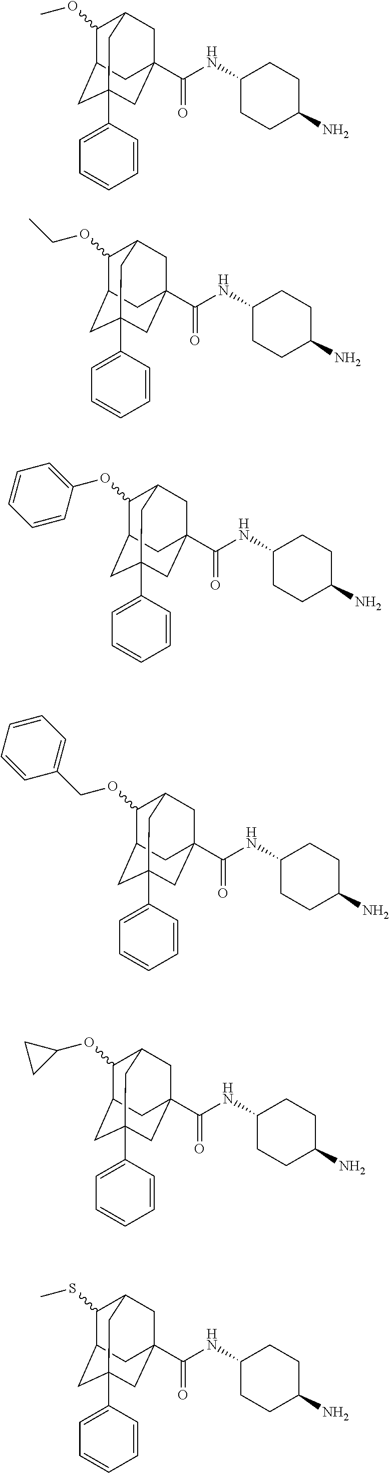 [SMILES: COC1C2CC3(C(=O)N[C@H]4CC[C@H](N)CC4)CC1CC(c1ccccc1)(C2)C3 ; CCOC1C2CC3(C(=O)N[C@H]4CC[C@H](N)CC4)CC1CC(c1ccccc1)(C2)C3 ; N[C@H]1CC[C@H](NC(=O)C23CC4CC(c5ccccc5)(CC(C2)C4Oc2ccccc2)C3)CC1 ; N[C@H]1CC[C@H](NC(=O)C23CC4CC(c5ccccc5)(CC(C2)C4OCc2ccccc2)C3)CC1 ; N[C@H]1CC[C@H](NC(=O)C23CC4CC(c5ccccc5)(CC(C2)C4OC2CC2)C3)CC1 ; CSC1C2CC3(C(=O)N[C@H]4CC[C@H](N)CC4)CC1CC(c1ccccc1)(C2)C3]

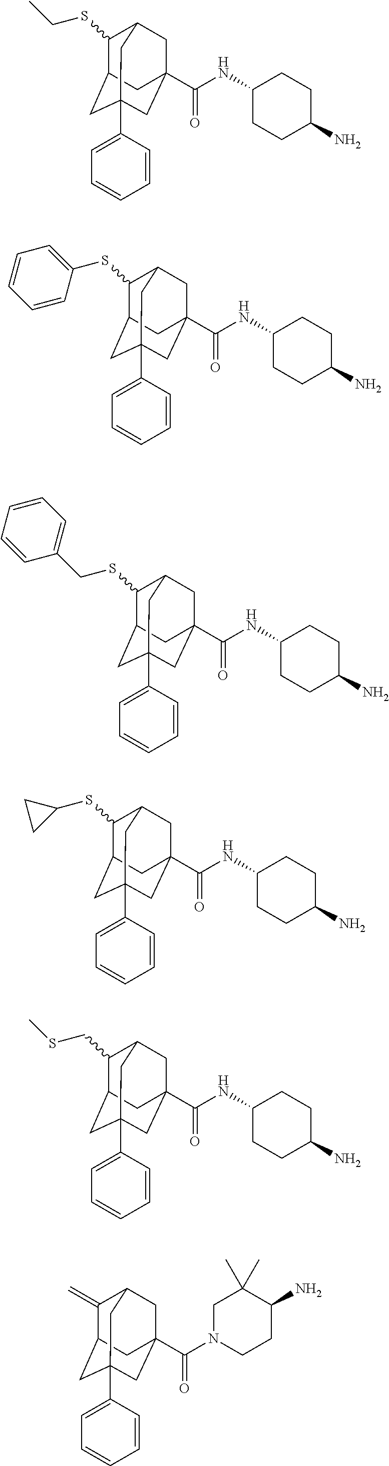 [SMILES: CCSC1C2CC3(C(=O)N[C@H]4CC[C@H](N)CC4)CC1CC(c1ccccc1)(C2)C3 ; N[C@H]1CC[C@H](NC(=O)C23CC4CC(c5ccccc5)(CC(C2)C4Sc2ccccc2)C3)CC1 ; N[C@H]1CC[C@H](NC(=O)C23CC4CC(c5ccccc5)(CC(C2)C4SCc2ccccc2)C3)CC1 ; N[C@H]1CC[C@H](NC(=O)C23CC4CC(c5ccccc5)(CC(C2)C4SC2CC2)C3)CC1 ; CSCC1C2CC3(C(=O)N[C@H]4CC[C@H](N)CC4)CC1CC(c1ccccc1)(C2)C3 ; C=C1C2CC3(C(=O)N4CC[C@H](N)C(C)(C)C4)CC1CC(c1ccccc1)(C2)C3]

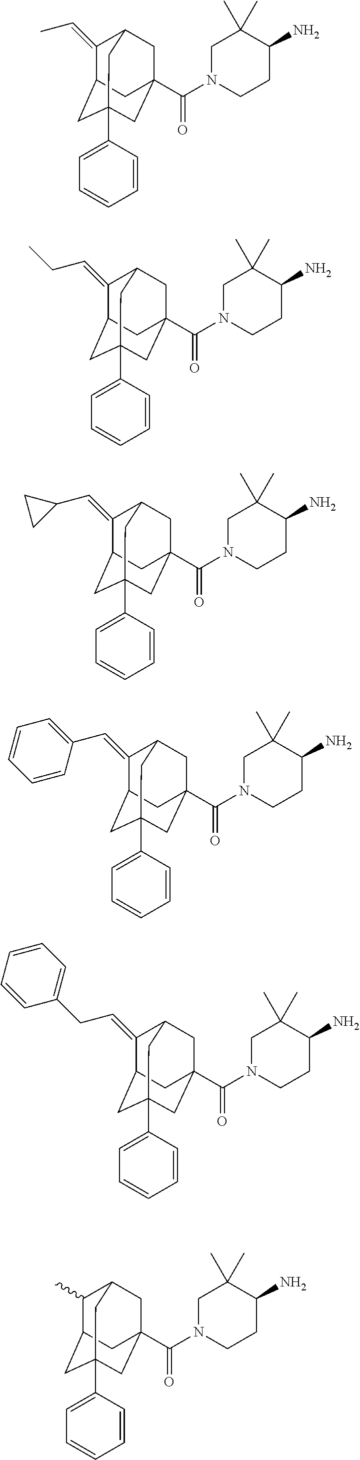 [SMILES: CC=C1C2CC3(C(=O)N4CC[C@H](N)C(C)(C)C4)CC1CC(c1ccccc1)(C2)C3 ; CCC=C1C2CC3(C(=O)N4CC[C@H](N)C(C)(C)C4)CC1CC(c1ccccc1)(C2)C3 ; CC1(C)CN(C(=O)C23CC4CC(c5ccccc5)(CC(C2)C4=CC2CC2)C3)CC[C@@H]1N ; CC1(C)CN(C(=O)C23CC4CC(c5ccccc5)(CC(C2)C4=Cc2ccccc2)C3)CC[C@@H]1N ; CC1(C)CN(C(=O)C23CC4CC(c5ccccc5)(CC(C2)C4=CCc2ccccc2)C3)CC[C@@H]1N ; CC1C2CC3(C(=O)N4CC[C@H](N)C(C)(C)C4)CC1CC(c1ccccc1)(C2)C3]

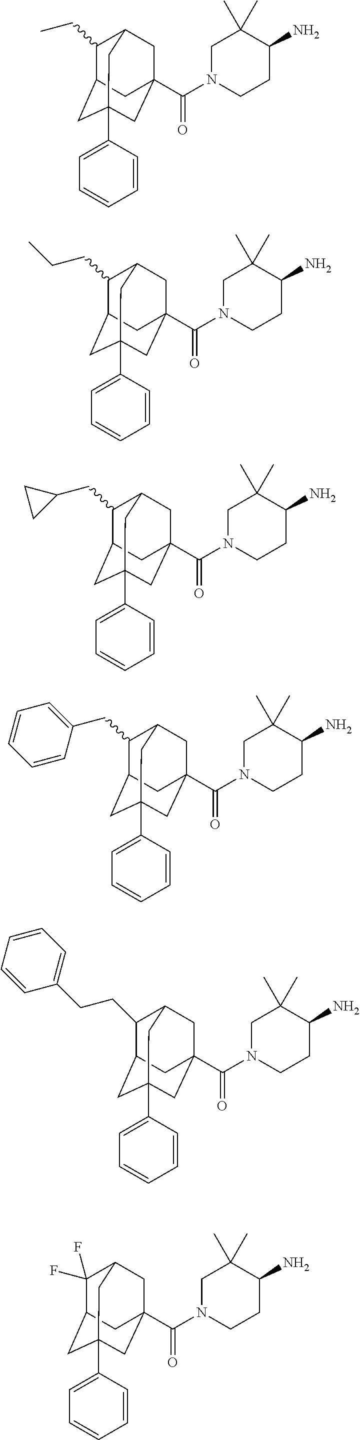 [SMILES: CCC1C2CC3(C(=O)N4CC[C@H](N)C(C)(C)C4)CC1CC(c1ccccc1)(C2)C3 ; CCCC1C2CC3(C(=O)N4CC[C@H](N)C(C)(C)C4)CC1CC(c1ccccc1)(C2)C3 ; CC1(C)CN(C(=O)C23CC4CC(c5ccccc5)(CC(C2)C4CC2CC2)C3)CC[C@@H]1N ; CC1(C)CN(C(=O)C23CC4CC(c5ccccc5)(CC(C2)C4Cc2ccccc2)C3)CC[C@@H]1N ; CC1(C)CN(C(=O)C23CC4CC(c5ccccc5)(CC(C2)C4CCc2ccccc2)C3)CC[C@@H]1N ; CC1(C)CN(C(=O)C23CC4CC(c5ccccc5)(CC(C2)C4(F)F)C3)CC[C@@H]1N]

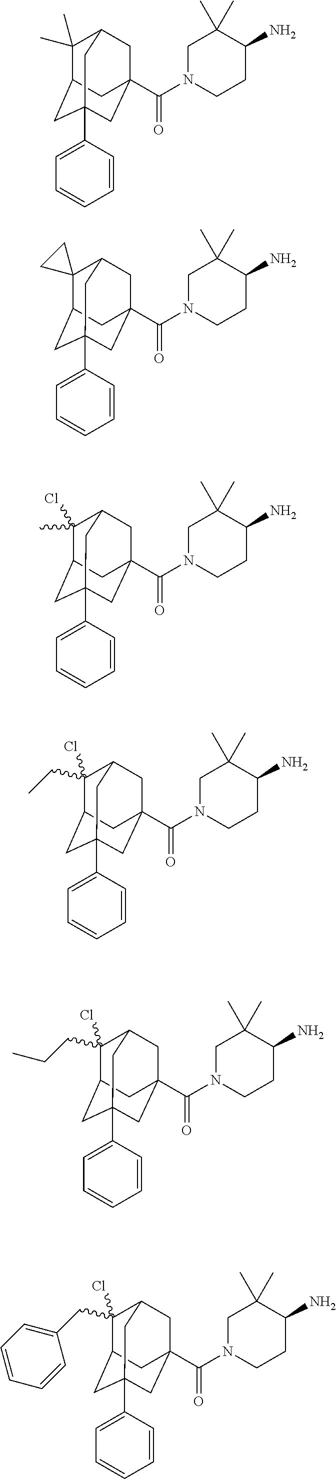

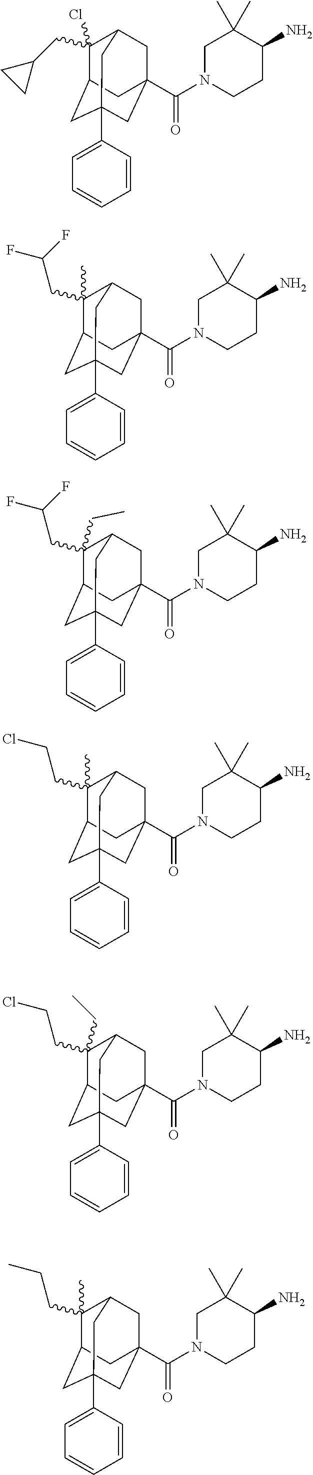

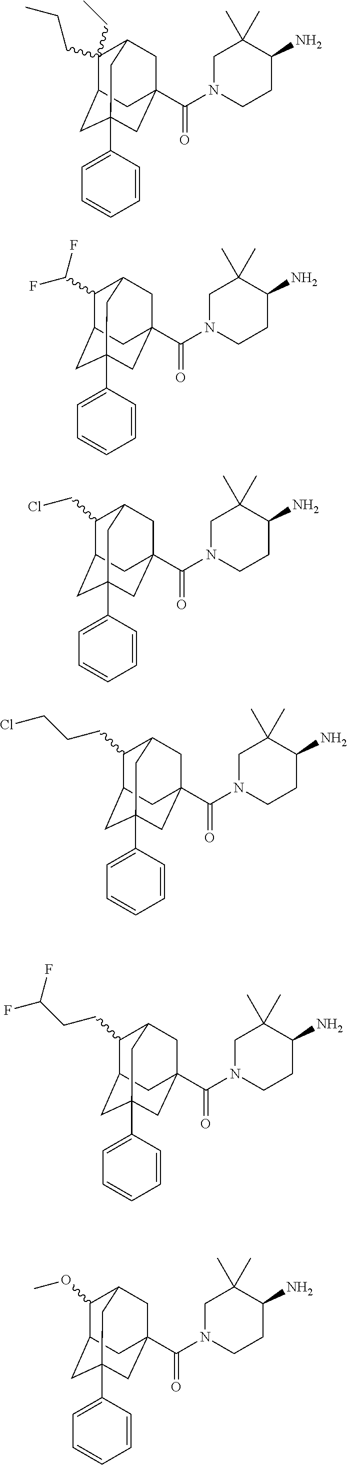

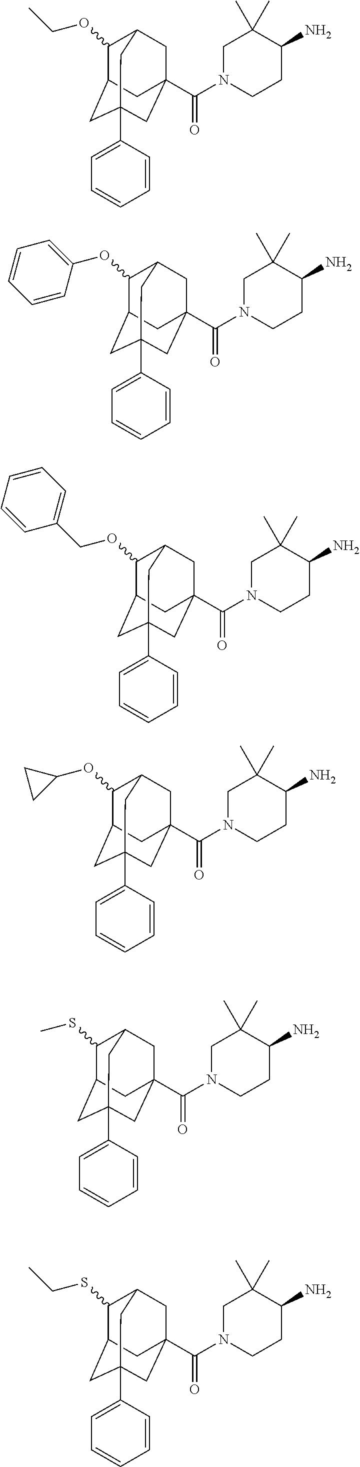 [SMILES: CCOC1C2CC3(C(=O)N4CC[C@H](N)C(C)(C)C4)CC1CC(c1ccccc1)(C2)C3 ; CC1(C)CN(C(=O)C23CC4CC(c5ccccc5)(CC(C2)C4Oc2ccccc2)C3)CC[C@@H]1N ; CC1(C)CN(C(=O)C23CC4CC(c5ccccc5)(CC(C2)C4OCc2ccccc2)C3)CC[C@@H]1N ; CC1(C)CN(C(=O)C23CC4CC(c5ccccc5)(CC(C2)C4OC2CC2)C3)CC[C@@H]1N ; CSC1C2CC3(C(=O)N4CC[C@H](N)C(C)(C)C4)CC1CC(c1ccccc1)(C2)C3 ; CCSC1C2CC3(C(=O)N4CC[C@H](N)C(C)(C)C4)CC1CC(c1ccccc1)(C2)C3]

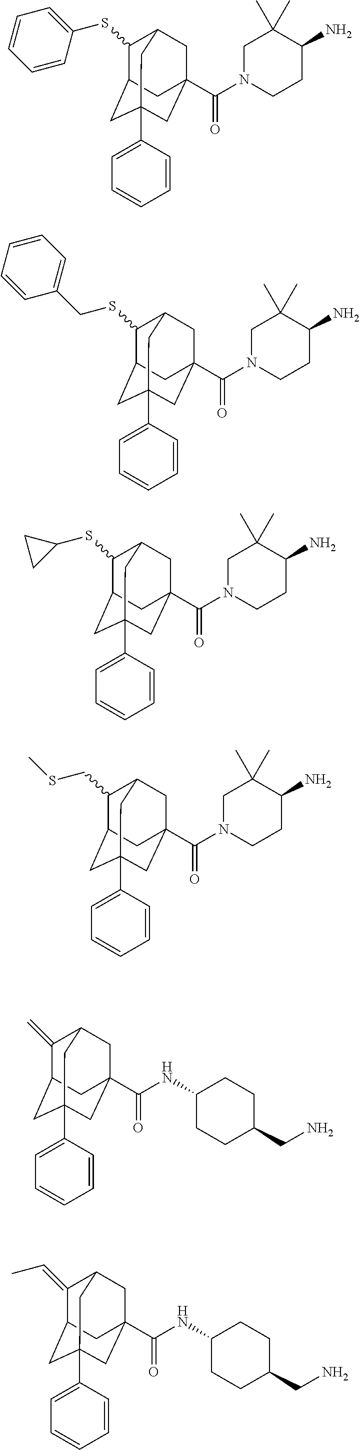 [SMILES: CC1(C)CN(C(=O)C23CC4CC(c5ccccc5)(CC(C2)C4Sc2ccccc2)C3)CC[C@@H]1N ; CC1(C)CN(C(=O)C23CC4CC(c5ccccc5)(CC(C2)C4SCc2ccccc2)C3)CC[C@@H]1N ; CC1(C)CN(C(=O)C23CC4CC(c5ccccc5)(CC(C2)C4SC2CC2)C3)CC[C@@H]1N ; CSCC1C2CC3(C(=O)N4CC[C@H](N)C(C)(C)C4)CC1CC(c1ccccc1)(C2)C3 ; C=C1C2CC3(C(=O)N[C@H]4CC[C@H](CN)CC4)CC1CC(c1ccccc1)(C2)C3 ; CC=C1C2CC3(C(=O)N[C@H]4CC[C@H](CN)CC4)CC1CC(c1ccccc1)(C2)C3]

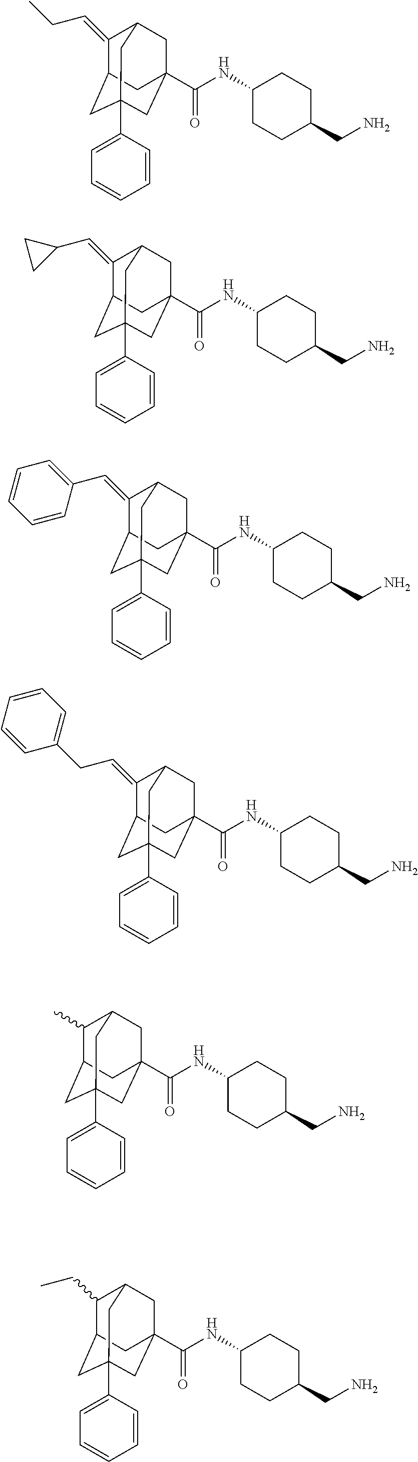 [SMILES: CCC=C1C2CC3(C(=O)N[C@H]4CC[C@H](CN)CC4)CC1CC(c1ccccc1)(C2)C3 ; NC[C@H]1CC[C@H](NC(=O)C23CC4CC(c5ccccc5)(CC(C2)C4=CC2CC2)C3)CC1 ; NC[C@H]1CC[C@H](NC(=O)C23CC4CC(c5ccccc5)(CC(C2)C4=Cc2ccccc2)C3)CC1 ; NC[C@H]1CC[C@H](NC(=O)C23CC4CC(c5ccccc5)(CC(C2)C4=CCc2ccccc2)C3)CC1 ; CC1C2CC3(C(=O)N[C@H]4CC[C@H](CN)CC4)CC1CC(c1ccccc1)(C2)C3 ; CCC1C2CC3(C(=O)N[C@H]4CC[C@H](CN)CC4)CC1CC(c1ccccc1)(C2)C3]

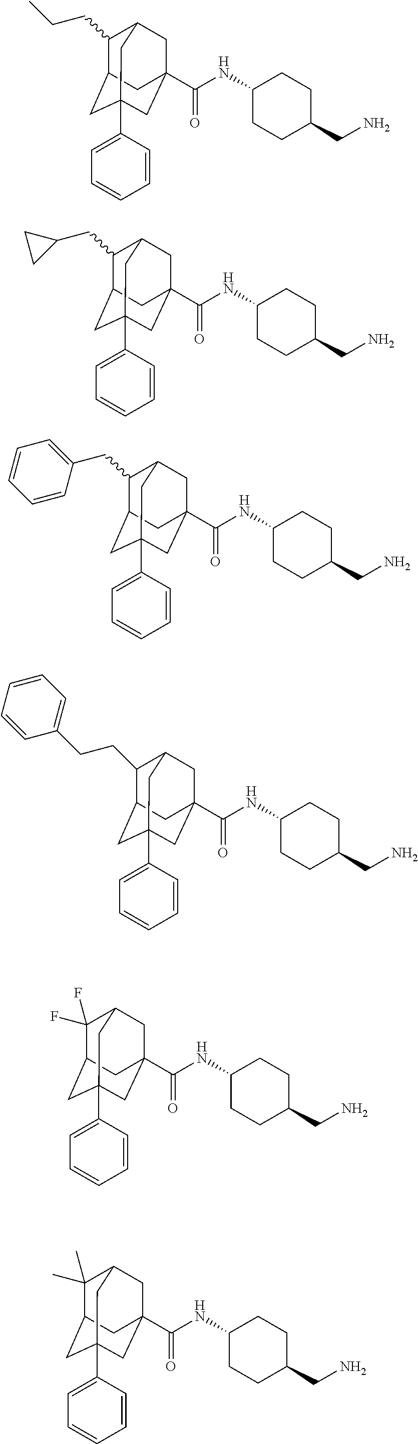

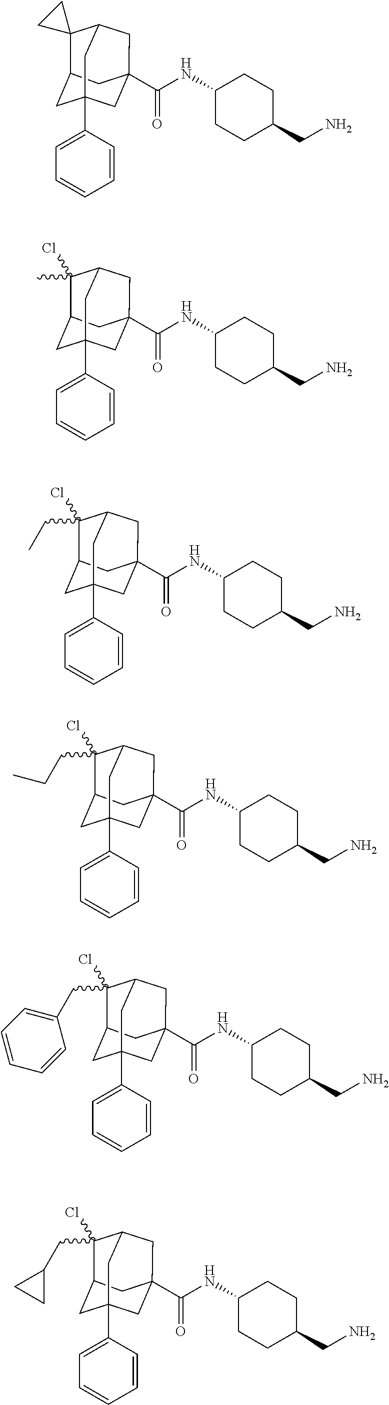

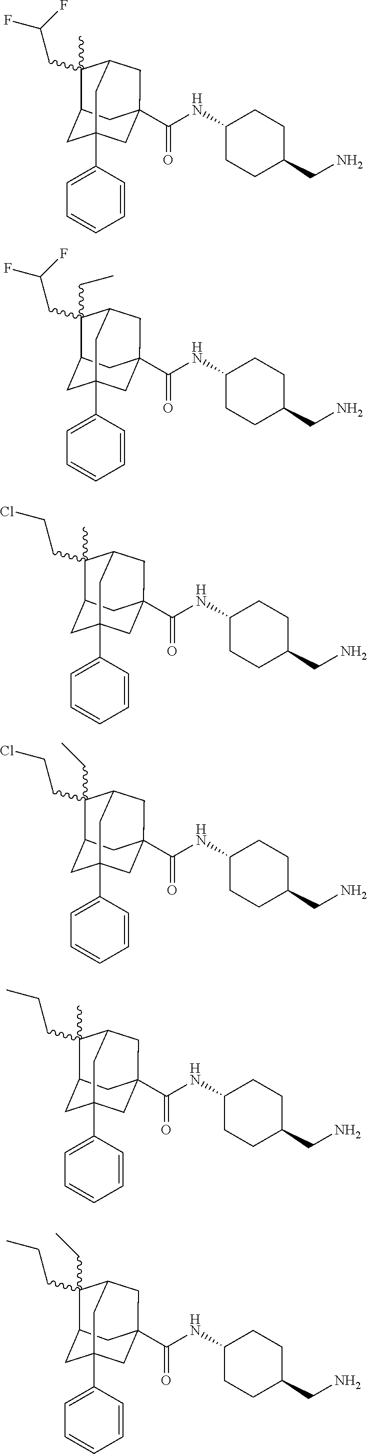

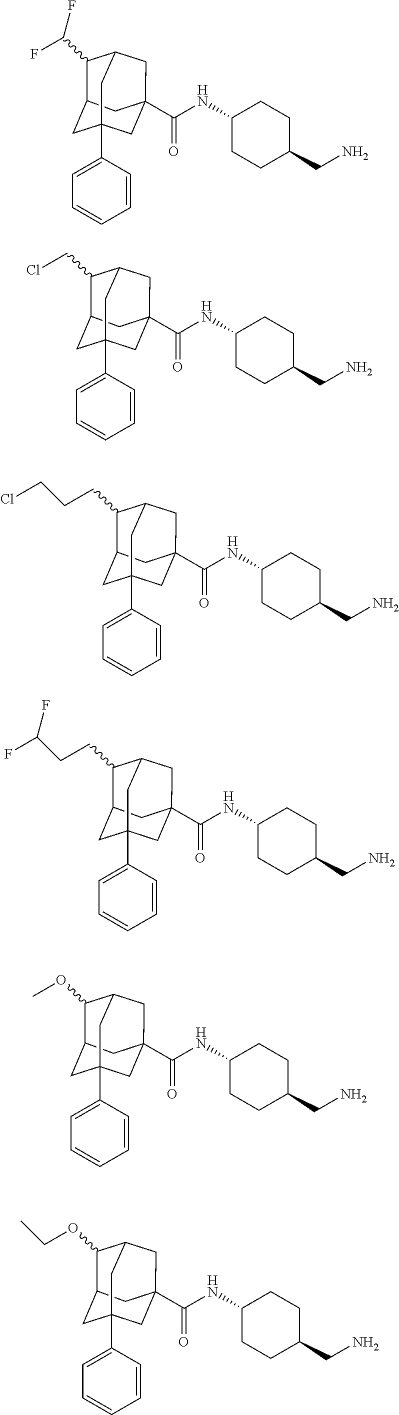

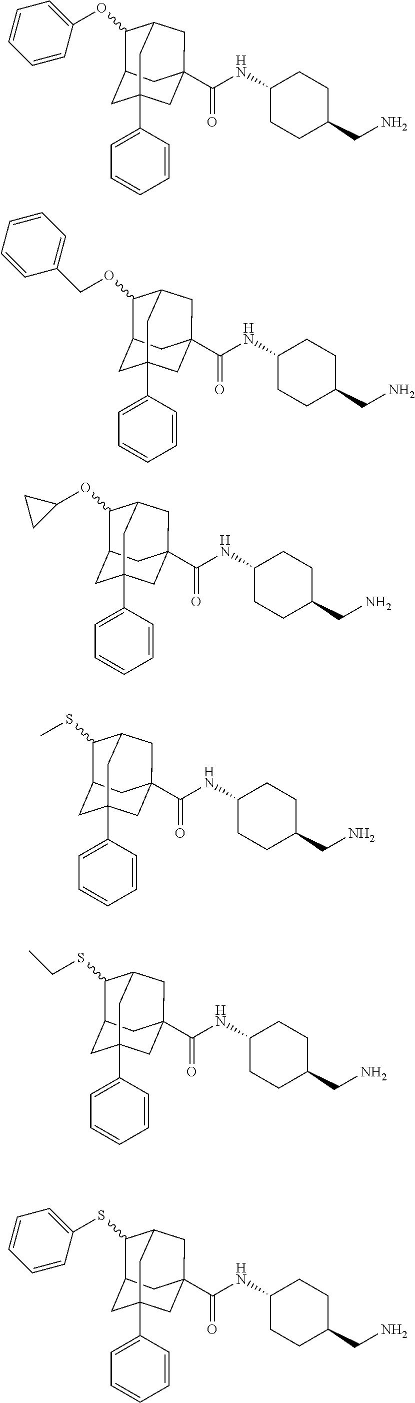

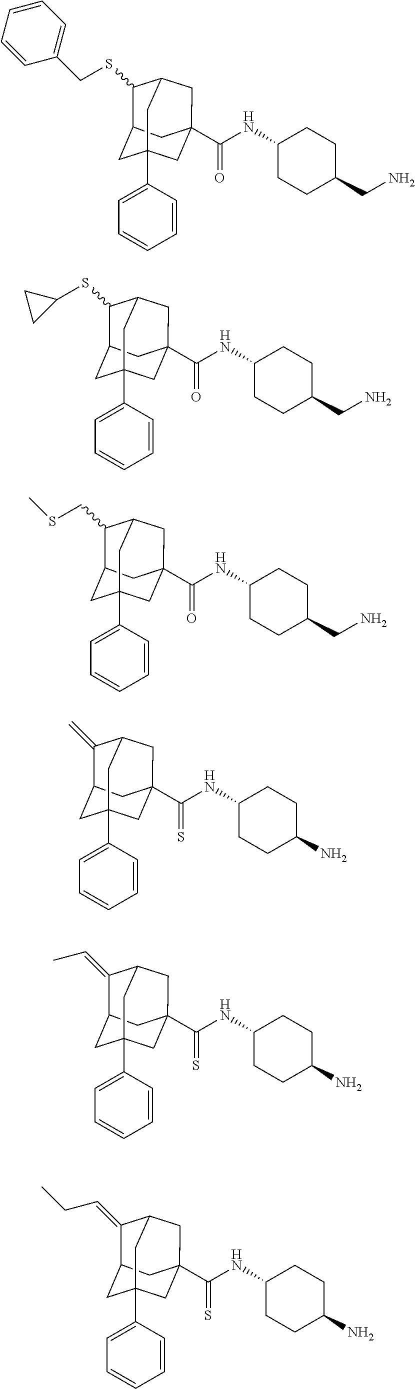

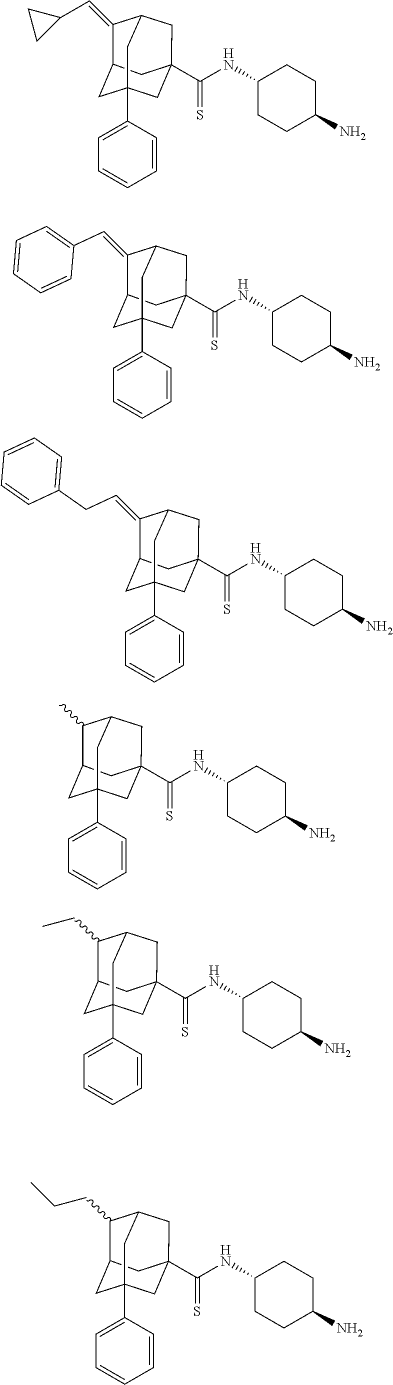

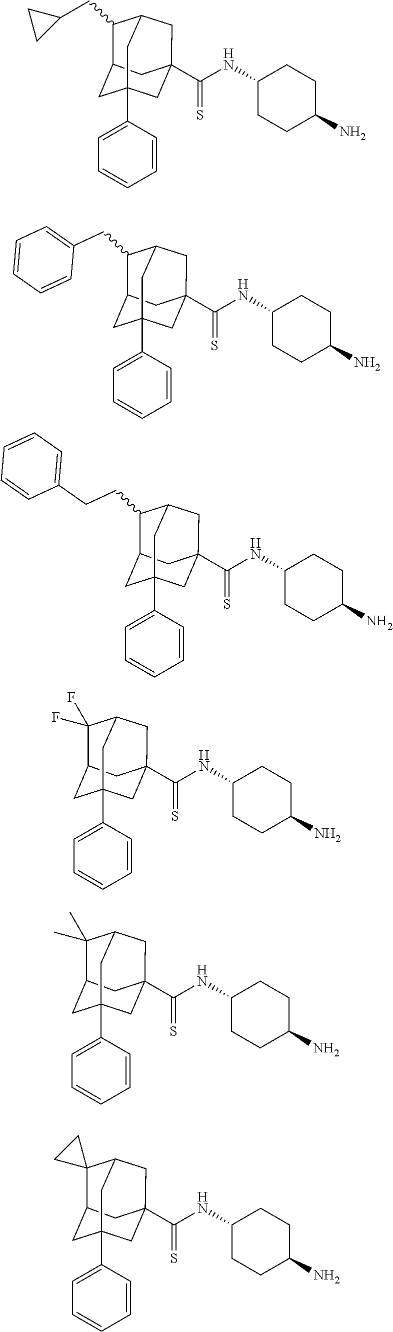

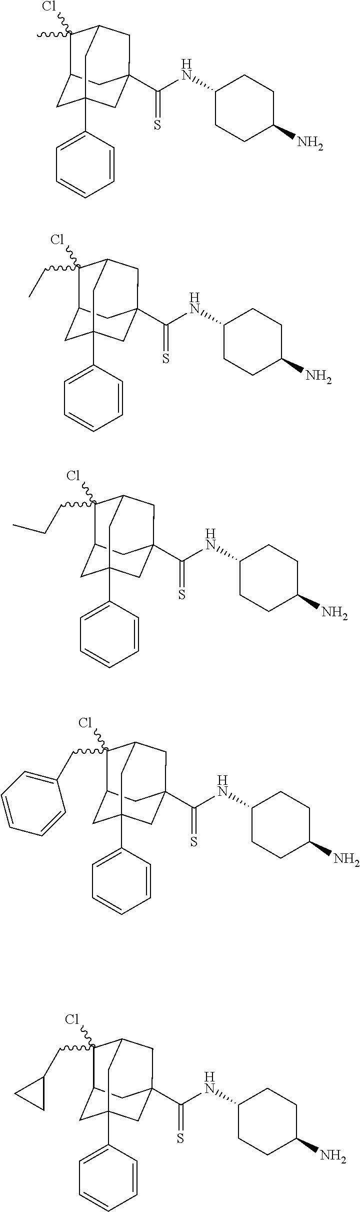

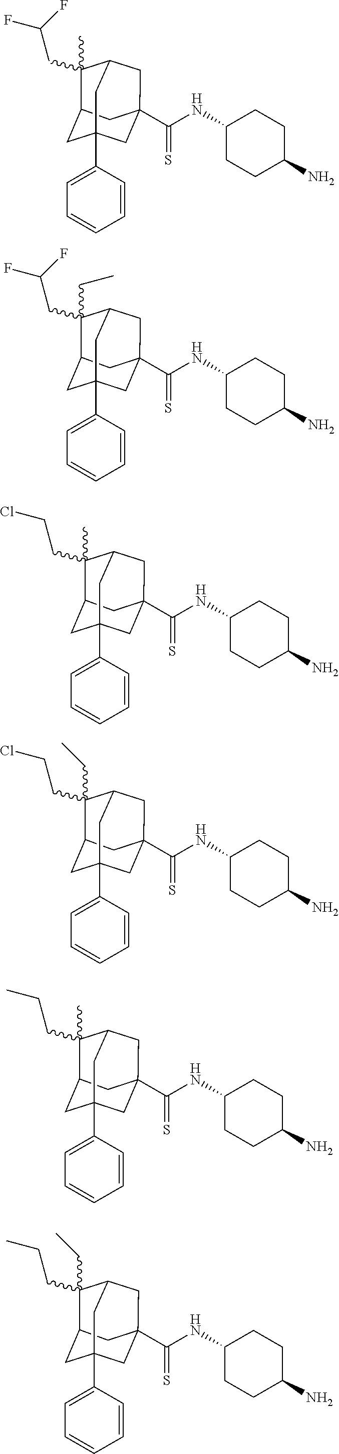

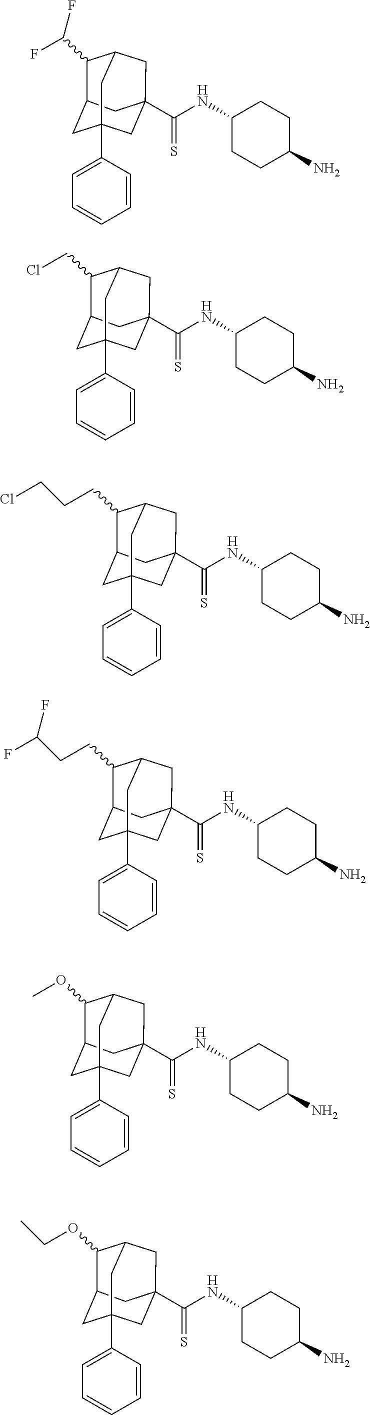

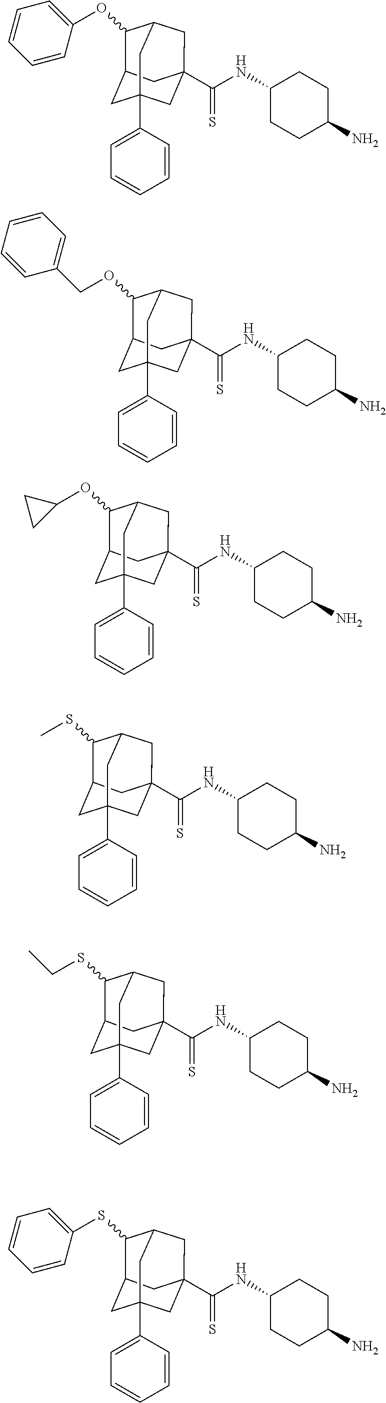

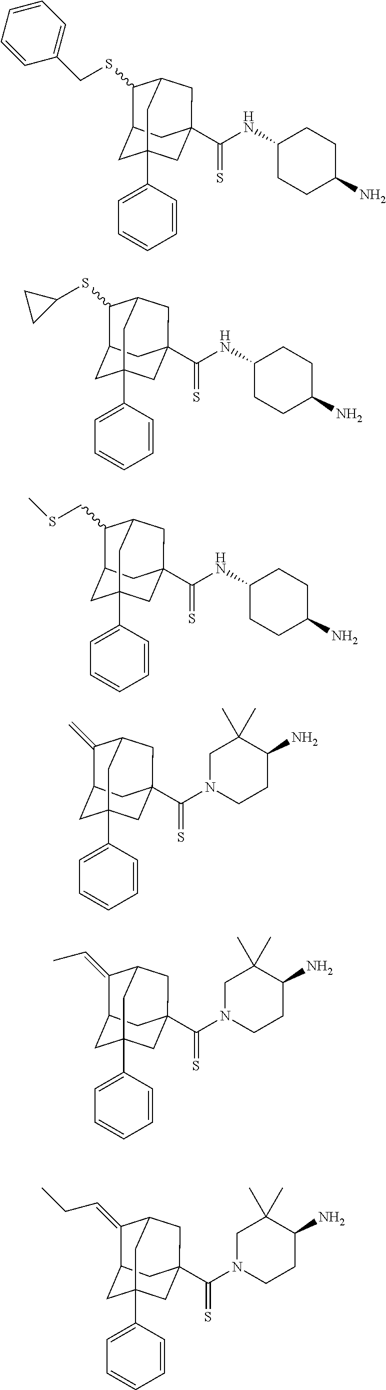

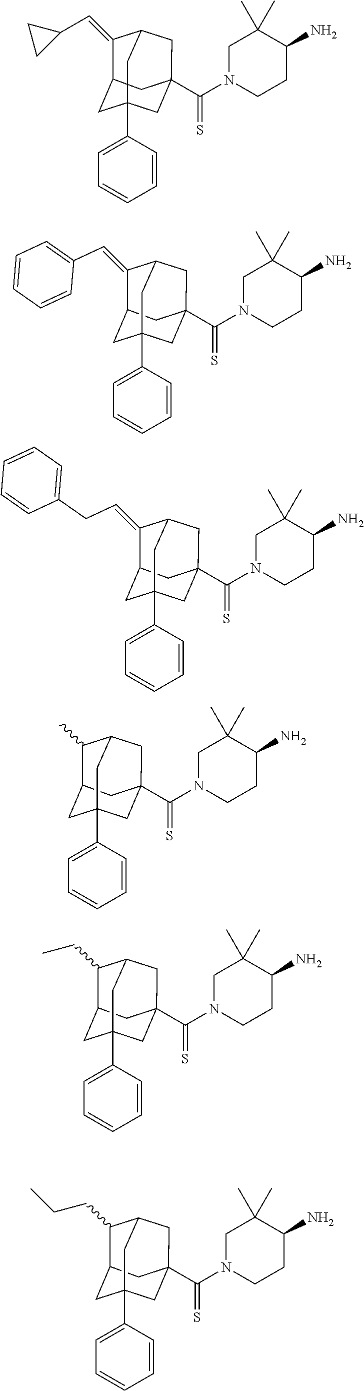

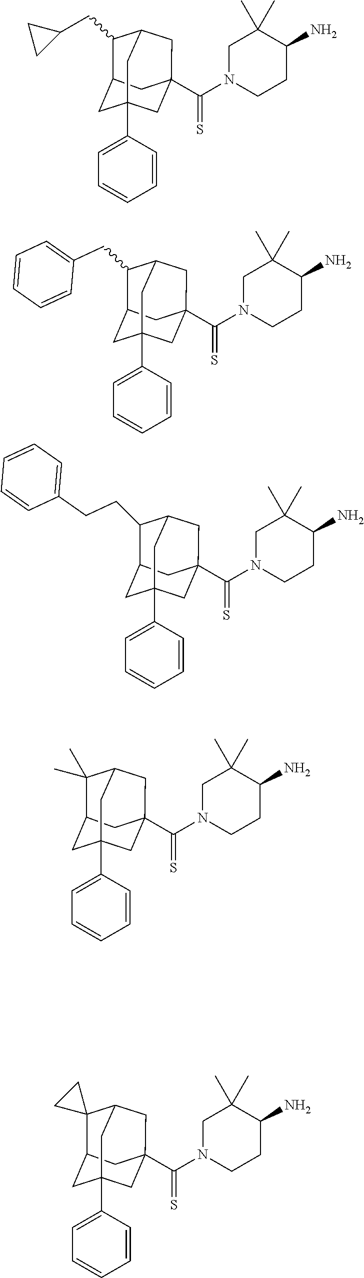 [SMILES: CC1(C)CN(C(=S)C23CC4CC(c5ccccc5)(CC(C2)C4CC2CC2)C3)CC[C@@H]1N ; CC1(C)CN(C(=S)C23CC4CC(c5ccccc5)(CC(C2)C4Cc2ccccc2)C3)CC[C@@H]1N ; CC1(C)CN(C(=S)C23CC4CC(c5ccccc5)(CC(C2)C4CCc2ccccc2)C3)CC[C@@H]1N ; CC1(C)C2CC3(C(=S)N4CC[C@H](N)C(C)(C)C4)CC1CC(c1ccccc1)(C2)C3 ; CC1(C)CN(C(=S)C23CC4CC(c5ccccc5)(CC(C2)C42CC2)C3)CC[C@@H]1N]

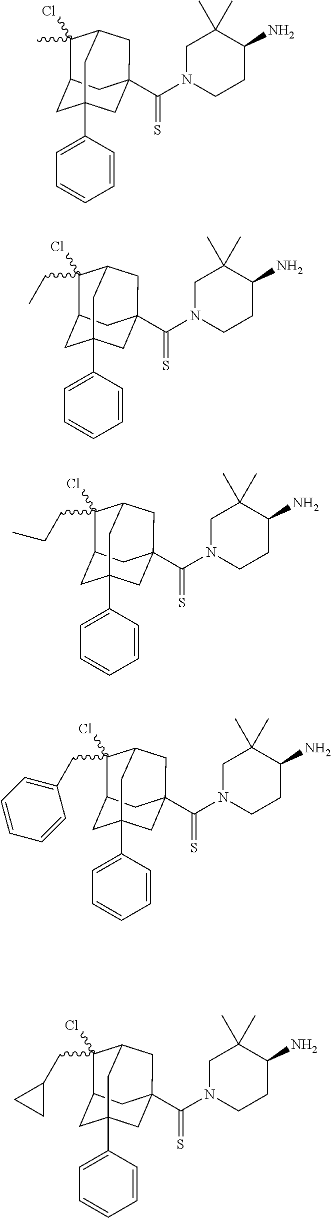

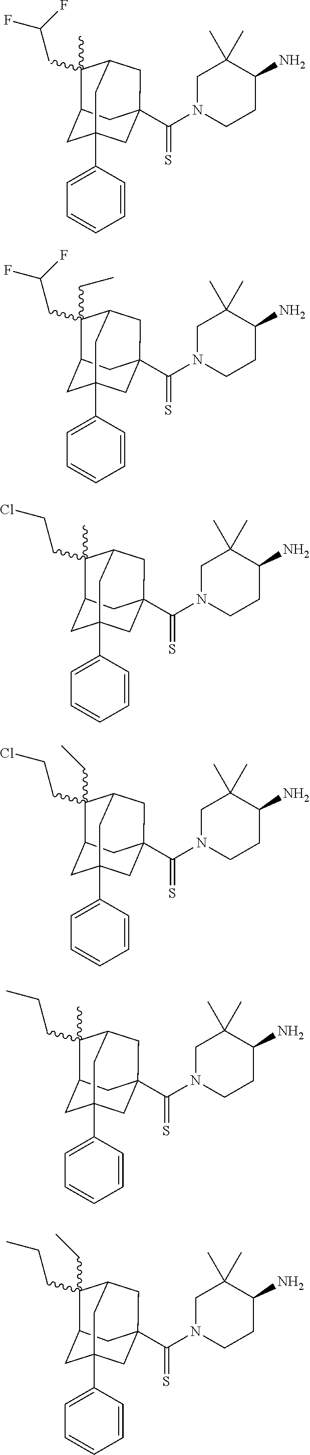

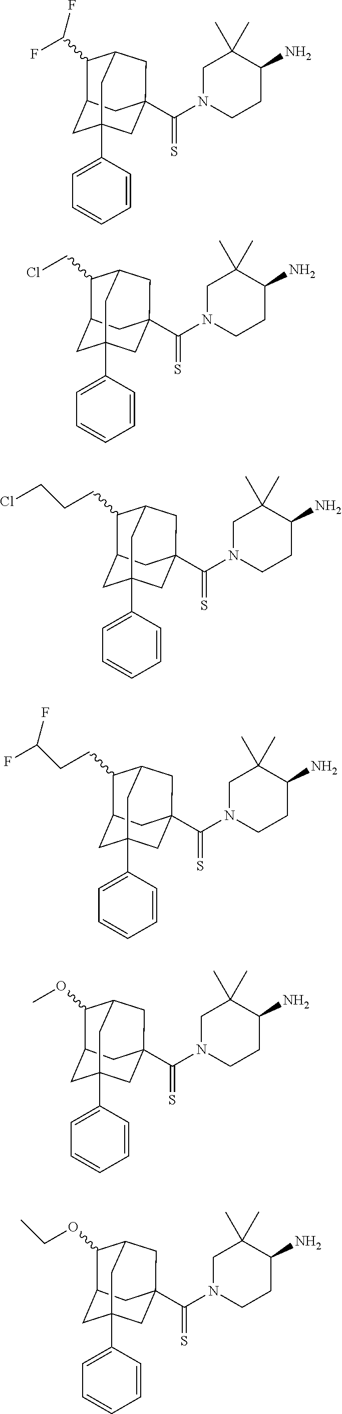

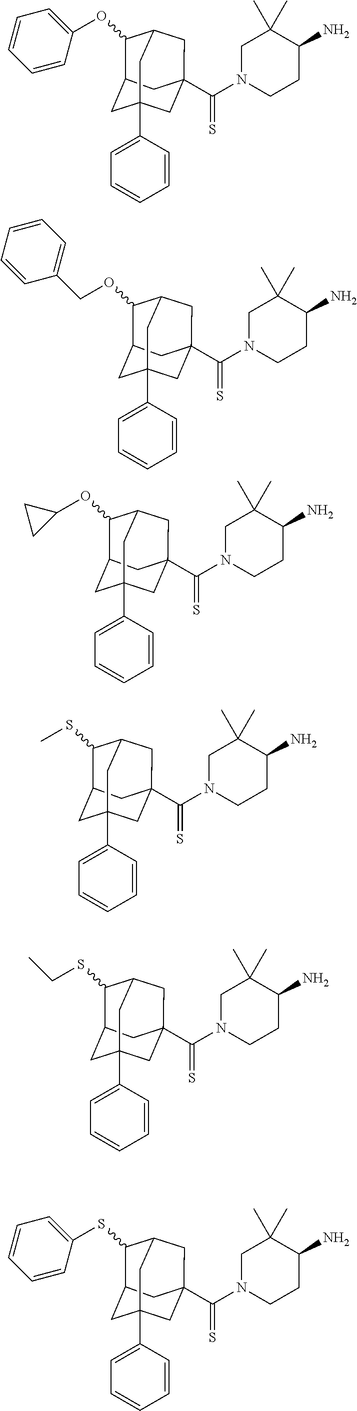

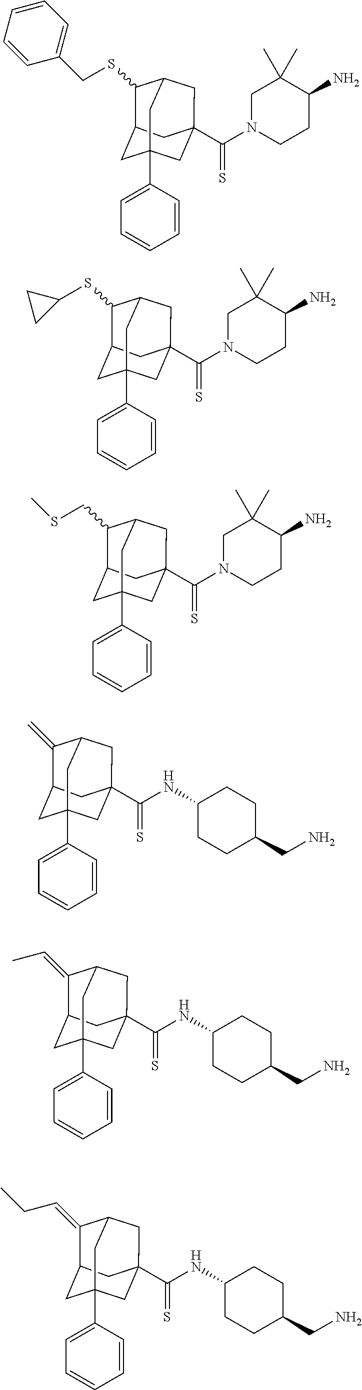 [SMILES: CC1(C)CN(C(=S)C23CC4CC(c5ccccc5)(CC(C2)C4SCc2ccccc2)C3)CC[C@@H]1N ; CC1(C)CN(C(=S)C23CC4CC(c5ccccc5)(CC(C2)C4SC2CC2)C3)CC[C@@H]1N ; CSCC1C2CC3(C(=S)N4CC[C@H](N)C(C)(C)C4)CC1CC(c1ccccc1)(C2)C3 ; C=C1C2CC3(C(=S)N[C@H]4CC[C@H](CN)CC4)CC1CC(c1ccccc1)(C2)C3 ; CC=C1C2CC3(C(=S)N[C@H]4CC[C@H](CN)CC4)CC1CC(c1ccccc1)(C2)C3 ; CCC=C1C2CC3(C(=S)N[C@H]4CC[C@H](CN)CC4)CC1CC(c1ccccc1)(C2)C3]

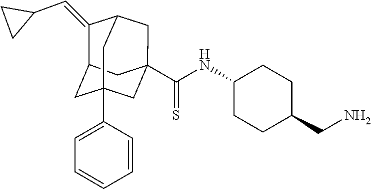 [SMILES: NC[C@H]1CC[C@H](NC(=S)C23CC4CC(c5ccccc5)(CC(C2)C4=CC2CC2)C3)CC1]

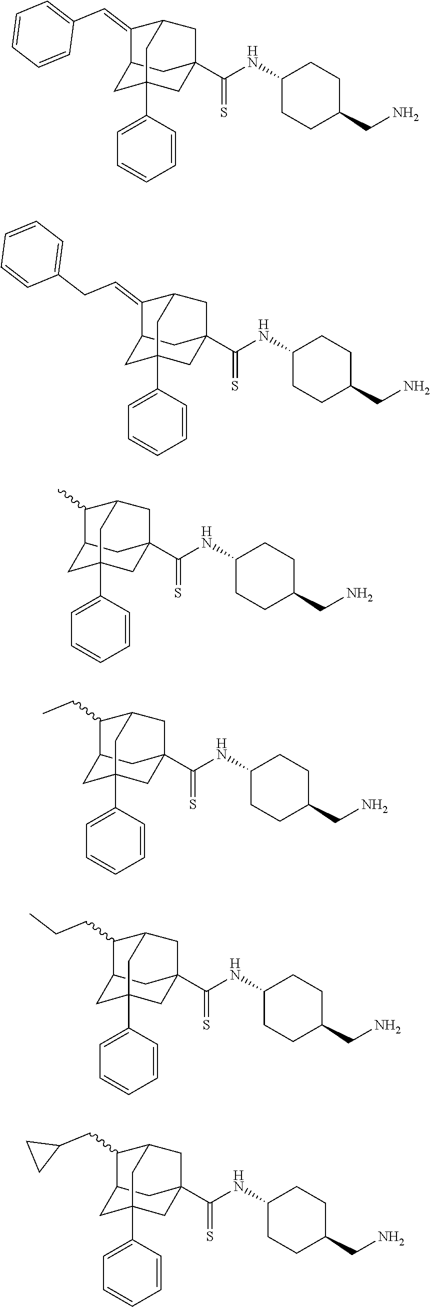 [SMILES: NC[C@H]1CC[C@H](NC(=S)C23CC4CC(c5ccccc5)(CC(C2)C4=Cc2ccccc2)C3)CC1 ; NC[C@H]1CC[C@H](NC(=S)C23CC4CC(c5ccccc5)(CC(C2)C4=CCc2ccccc2)C3)CC1 ; CC1C2CC3(C(=S)N[C@H]4CC[C@H](CN)CC4)CC1CC(c1ccccc1)(C2)C3 ; CCC1C2CC3(C(=S)N[C@H]4CC[C@H](CN)CC4)CC1CC(c1ccccc1)(C2)C3 ; CCCC1C2CC3(C(=S)N[C@H]4CC[C@H](CN)CC4)CC1CC(c1ccccc1)(C2)C3 ; NC[C@H]1CC[C@H](NC(=S)C23CC4CC(c5ccccc5)(CC(C2)C4CC2CC2)C3)CC1]

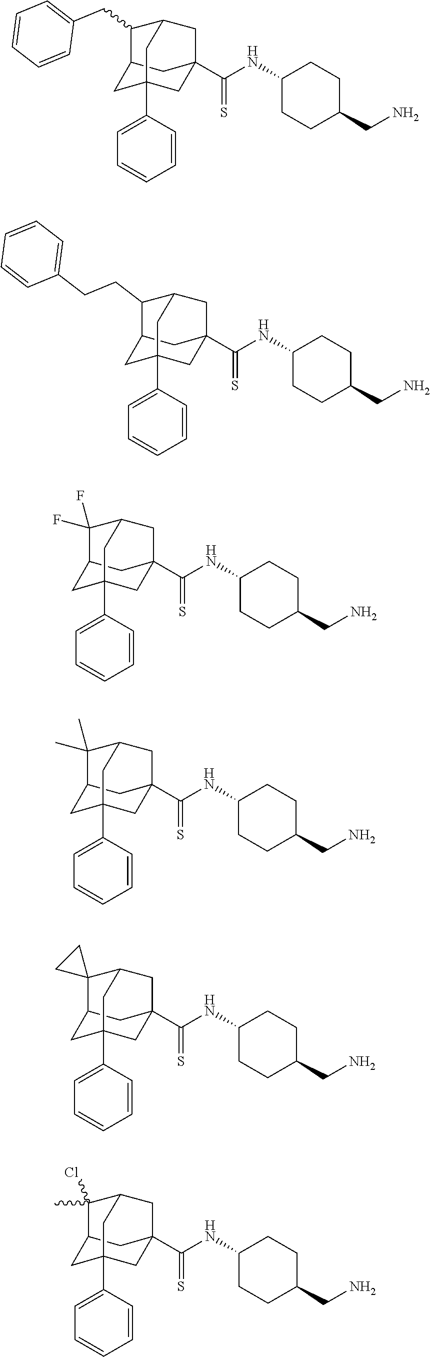

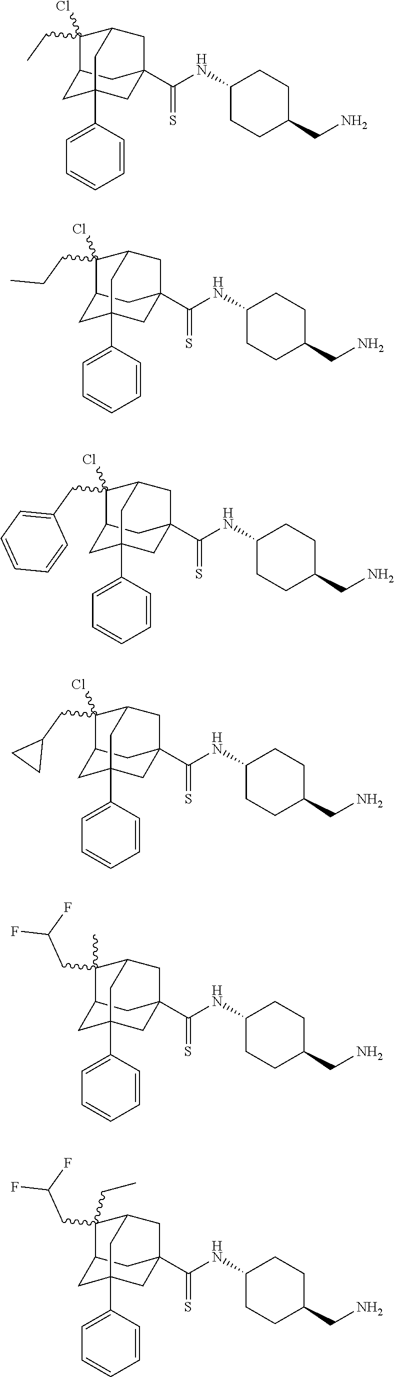

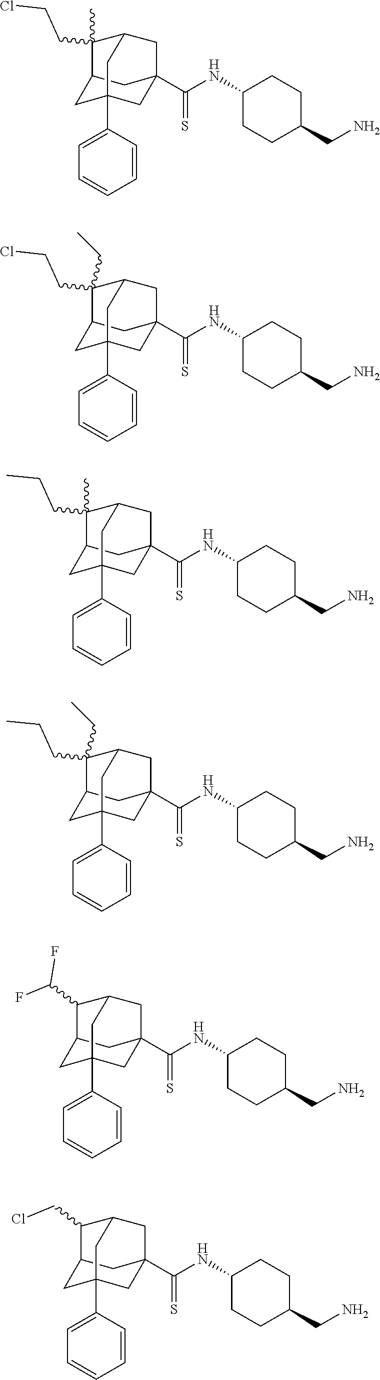

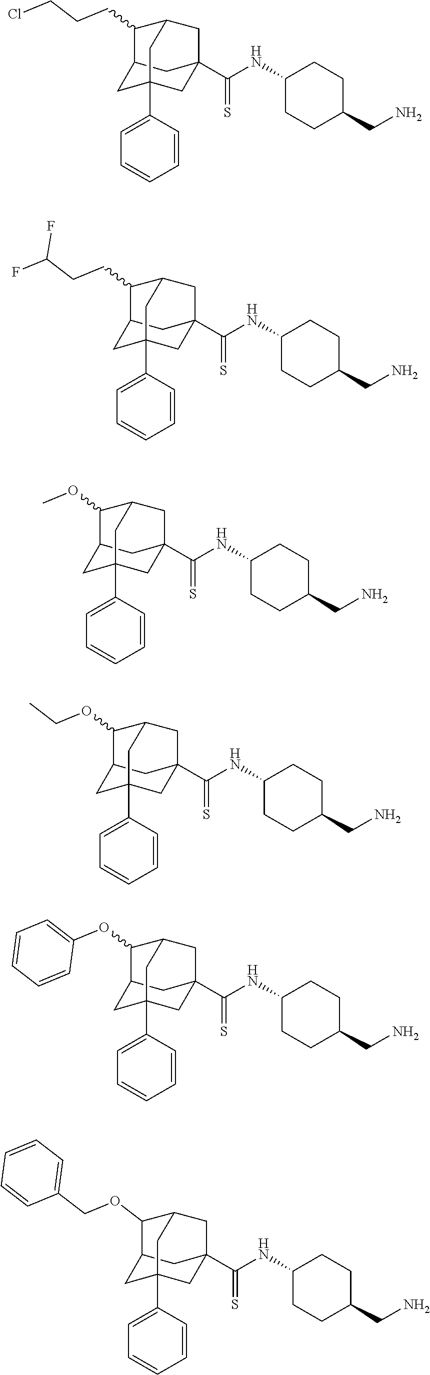 [SMILES: NC[C@H]1CC[C@H](NC(=S)C23CC4CC(c5ccccc5)(CC(C2)C4CCCCl)C3)CC1 ; NC[C@H]1CC[C@H](NC(=S)C23CC4CC(c5ccccc5)(CC(C2)C4CCC(F)F)C3)CC1 ; COC1C2CC3(C(=S)N[C@H]4CC[C@H](CN)CC4)CC1CC(c1ccccc1)(C2)C3 ; CCOC1C2CC3(C(=S)N[C@H]4CC[C@H](CN)CC4)CC1CC(c1ccccc1)(C2)C3 ; NC[C@H]1CC[C@H](NC(=S)C23CC4CC(c5ccccc5)(CC(C2)C4Oc2ccccc2)C3)CC1 ; NC[C@H]1CC[C@H](NC(=S)C23CC4CC(c5ccccc5)(CC(C2)C4OCc2ccccc2)C3)CC1]

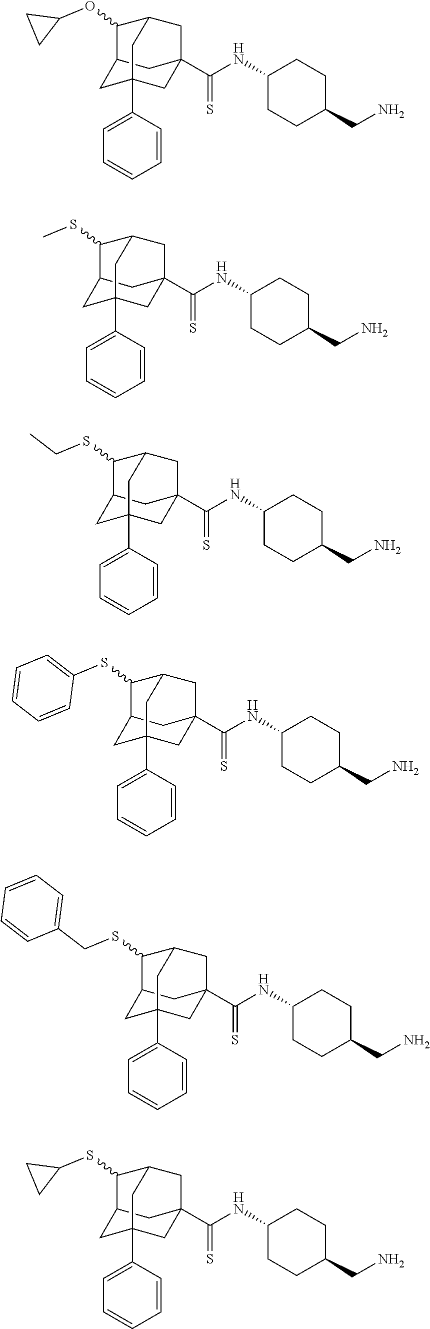 [SMILES: NC[C@H]1CC[C@H](NC(=S)C23CC4CC(c5ccccc5)(CC(C2)C4OC2CC2)C3)CC1 ; CSC1C2CC3(C(=S)N[C@H]4CC[C@H](CN)CC4)CC1CC(c1ccccc1)(C2)C3 ; CCSC1C2CC3(C(=S)N[C@H]4CC[C@H](CN)CC4)CC1CC(c1ccccc1)(C2)C3 ; NC[C@H]1CC[C@H](NC(=S)C23CC4CC(c5ccccc5)(CC(C2)C4Sc2ccccc2)C3)CC1 ; NC[C@H]1CC[C@H](NC(=S)C23CC4CC(c5ccccc5)(CC(C2)C4SCc2ccccc2)C3)CC1 ; NC[C@H]1CC[C@H](NC(=S)C23CC4CC(c5ccccc5)(CC(C2)C4SC2CC2)C3)CC1]

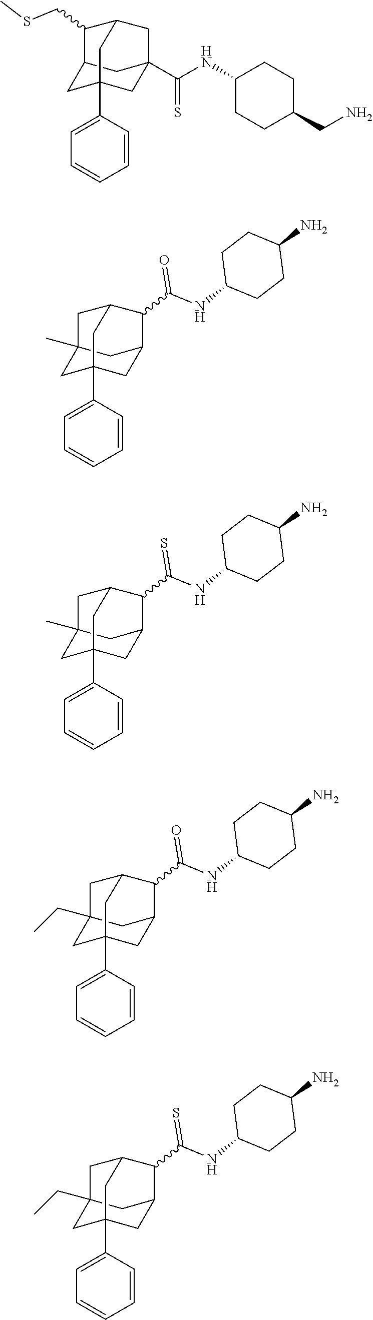 [SMILES: CSCC1C2CC3(C(=S)N[C@H]4CC[C@H](CN)CC4)CC1CC(c1ccccc1)(C2)C3 ; CC12CC3CC(c4ccccc4)(CC(C1)C3C(=O)N[C@H]1CC[C@H](N)CC1)C2 ; CC12CC3CC(c4ccccc4)(CC(C1)C3C(=S)N[C@H]1CC[C@H](N)CC1)C2 ; CCC12CC3CC(c4ccccc4)(CC(C1)C3C(=O)N[C@H]1CC[C@H](N)CC1)C2 ; CCC12CC3CC(c4ccccc4)(CC(C1)C3C(=S)N[C@H]1CC[C@H](N)CC1)C2]

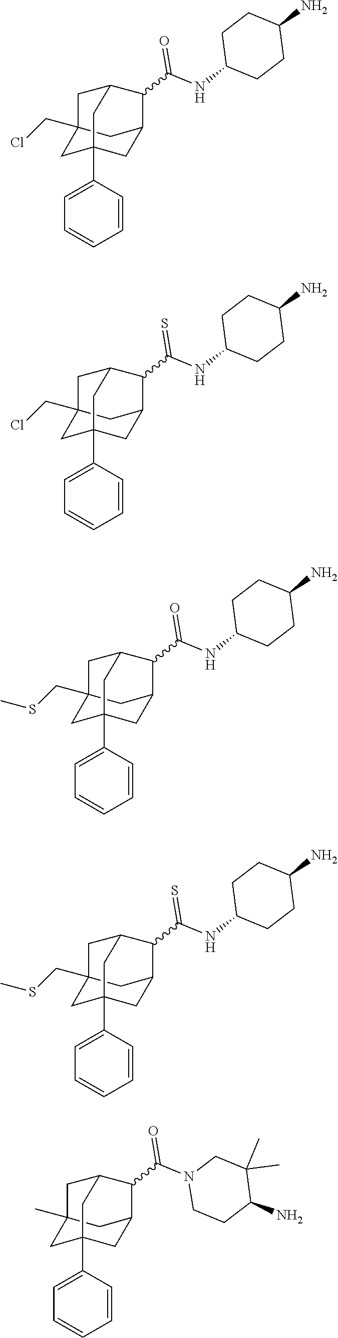 [SMILES: N[C@H]1CC[C@H](NC(=O)C2C3CC4(CCl)CC2CC(c2ccccc2)(C3)C4)CC1 ; N[C@H]1CC[C@H](NC(=S)C2C3CC4(CCl)CC2CC(c2ccccc2)(C3)C4)CC1 ; CSCC12CC3CC(c4ccccc4)(CC(C1)C3C(=O)N[C@H]1CC[C@H](N)CC1)C2 ; CSCC12CC3CC(c4ccccc4)(CC(C1)C3C(=S)N[C@H]1CC[C@H](N)CC1)C2 ; CC12CC3CC(c4ccccc4)(CC(C1)C3C(=O)N1CC[C@H](N)C(C)(C)C1)C2]

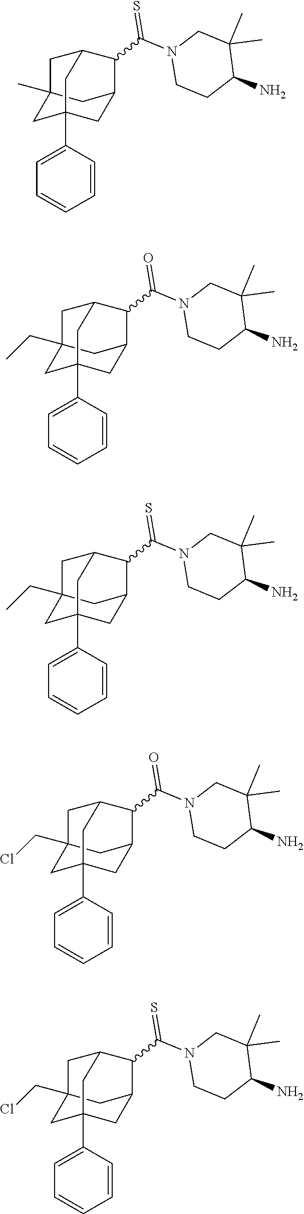 [SMILES: CC12CC3CC(c4ccccc4)(CC(C1)C3C(=S)N1CC[C@H](N)C(C)(C)C1)C2 ; CCC12CC3CC(c4ccccc4)(CC(C1)C3C(=O)N1CC[C@H](N)C(C)(C)C1)C2 ; CCC12CC3CC(c4ccccc4)(CC(C1)C3C(=S)N1CC[C@H](N)C(C)(C)C1)C2 ; CC1(C)CN(C(=O)C2C3CC4(CCl)CC2CC(c2ccccc2)(C3)C4)CC[C@@H]1N ; CC1(C)CN(C(=S)C2C3CC4(CCl)CC2CC(c2ccccc2)(C3)C4)CC[C@@H]1N]

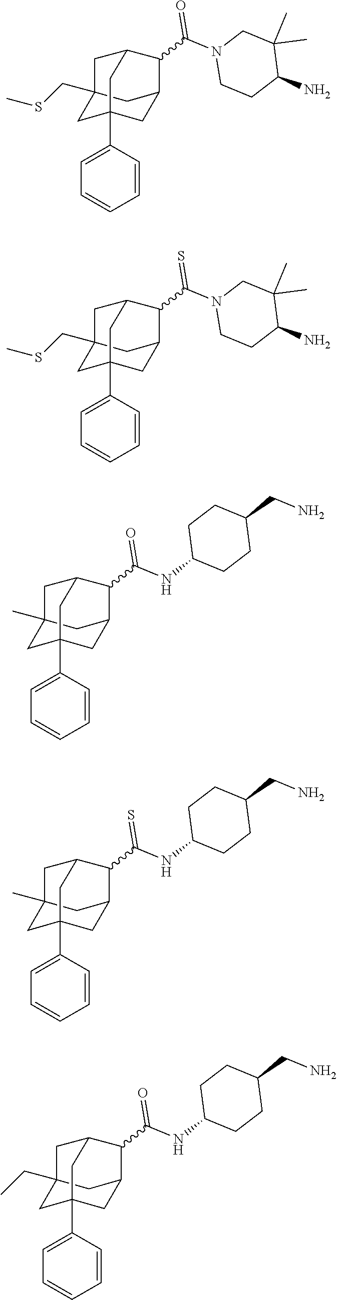 [SMILES: CSCC12CC3CC(c4ccccc4)(CC(C1)C3C(=O)N1CC[C@H](N)C(C)(C)C1)C2 ; CSCC12CC3CC(c4ccccc4)(CC(C1)C3C(=S)N1CC[C@H](N)C(C)(C)C1)C2 ; CC12CC3CC(c4ccccc4)(CC(C1)C3C(=O)N[C@H]1CC[C@H](CN)CC1)C2 ; CC12CC3CC(c4ccccc4)(CC(C1)C3C(=S)N[C@H]1CC[C@H](CN)CC1)C2 ; CCC12CC3CC(c4ccccc4)(CC(C1)C3C(=O)N[C@H]1CC[C@H](CN)CC1)C2]

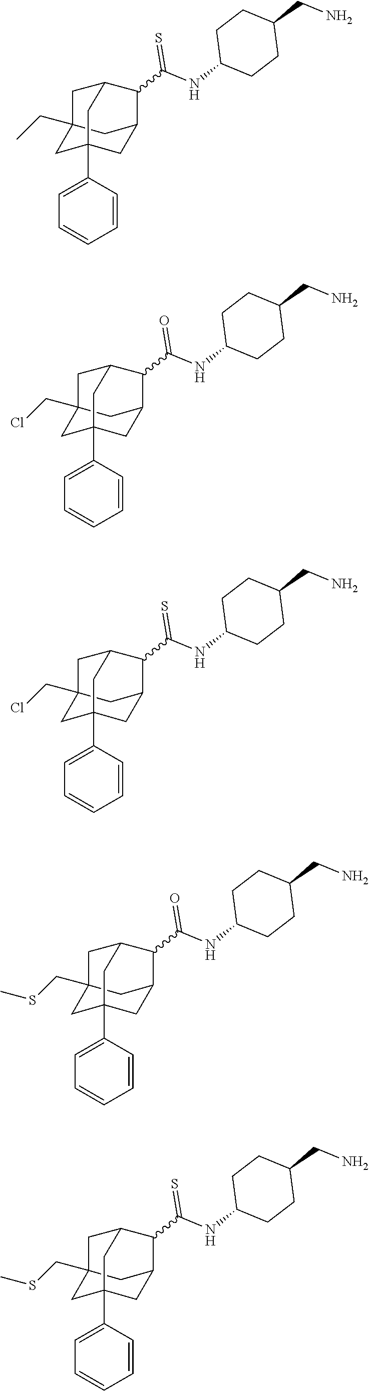 [SMILES: CCC12CC3CC(c4ccccc4)(CC(C1)C3C(=S)N[C@H]1CC[C@H](CN)CC1)C2 ; NC[C@H]1CC[C@H](NC(=O)C2C3CC4(CCl)CC2CC(c2ccccc2)(C3)C4)CC1 ; NC[C@H]1CC[C@H](NC(=S)C2C3CC4(CCl)CC2CC(c2ccccc2)(C3)C4)CC1 ; CSCC12CC3CC(c4ccccc4)(CC(C1)C3C(=O)N[C@H]1CC[C@H](CN)CC1)C2 ; CSCC12CC3CC(c4ccccc4)(CC(C1)C3C(=S)N[C@H]1CC[C@H](CN)CC1)C2]

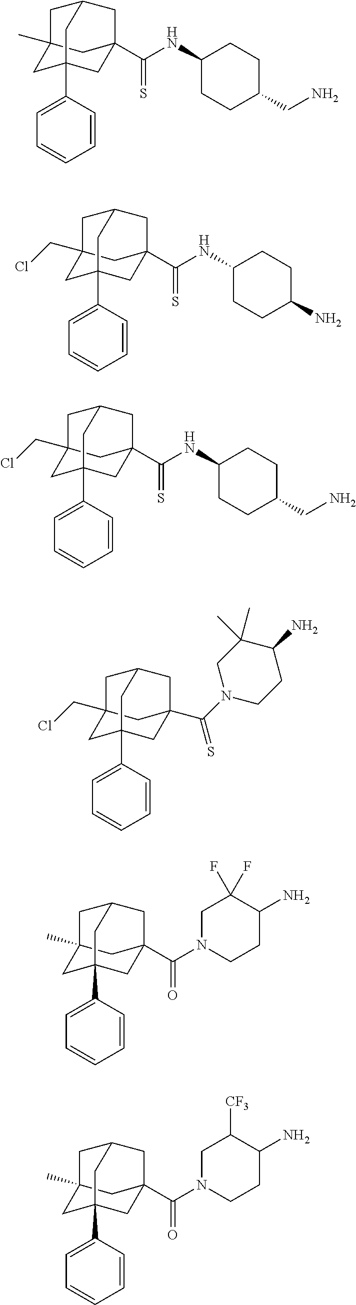

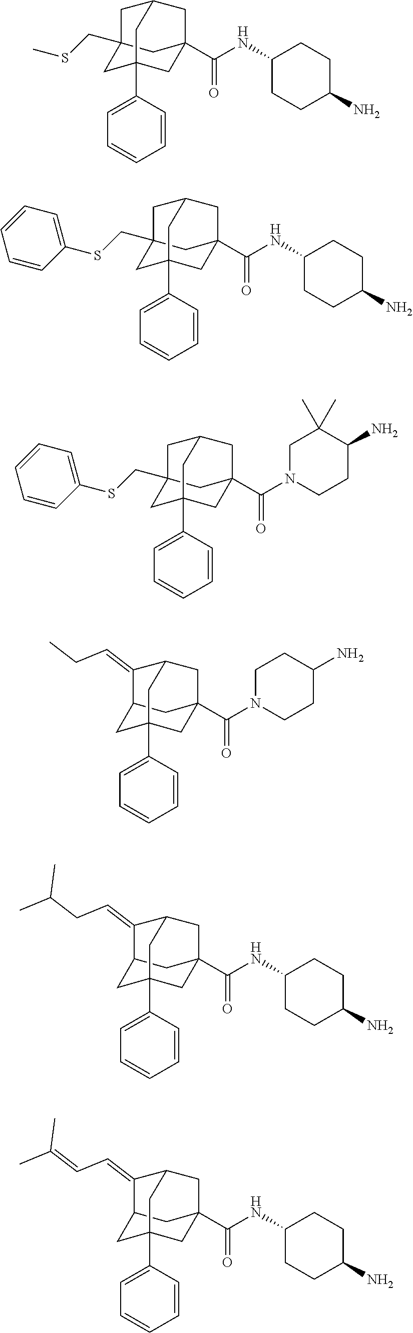 [SMILES: CSCC12CC3CC(C(=O)N[C@H]4CC[C@H](N)CC4)(C1)CC(c1ccccc1)(C3)C2 ; N[C@H]1CC[C@H](NC(=O)C23CC4CC(CSc5ccccc5)(C2)CC(c2ccccc2)(C4)C3)CC1 ; CC1(C)CN(C(=O)C23CC4CC(CSc5ccccc5)(C2)CC(c2ccccc2)(C4)C3)CC[C@@H]1N ; CCC=C1C2CC3(C(=O)N4CCC(N)CC4)CC1CC(c1ccccc1)(C2)C3 ; CC(C)CC=C1C2CC3(C(=O)N[C@H]4CC[C@H](N)CC4)CC1CC(c1ccccc1)(C2)C3 ; CC(C)=CC=C1C2CC3(C(=O)N[C@H]4CC[C@H](N)CC4)CC1CC(c1ccccc1)(C2)C3]

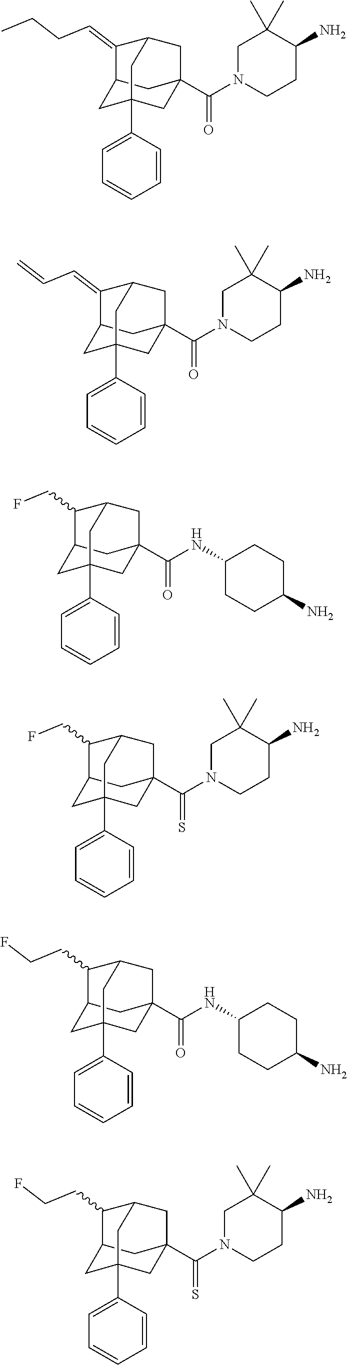 [SMILES: CCCC=C1C2CC3(C(=O)N4CC[C@H](N)C(C)(C)C4)CC1CC(c1ccccc1)(C2)C3 ; C=CC=C1C2CC3(C(=O)N4CC[C@H](N)C(C)(C)C4)CC1CC(c1ccccc1)(C2)C3 ; N[C@H]1CC[C@H](NC(=O)C23CC4CC(c5ccccc5)(CC(C2)C4CF)C3)CC1 ; CC1(C)CN(C(=S)C23CC4CC(c5ccccc5)(CC(C2)C4CF)C3)CC[C@@H]1N ; N[C@H]1CC[C@H](NC(=O)C23CC4CC(c5ccccc5)(CC(C2)C4CCF)C3)CC1 ; CC1(C)CN(C(=S)C23CC4CC(c5ccccc5)(CC(C2)C4CCF)C3)CC[C@@H]1N]

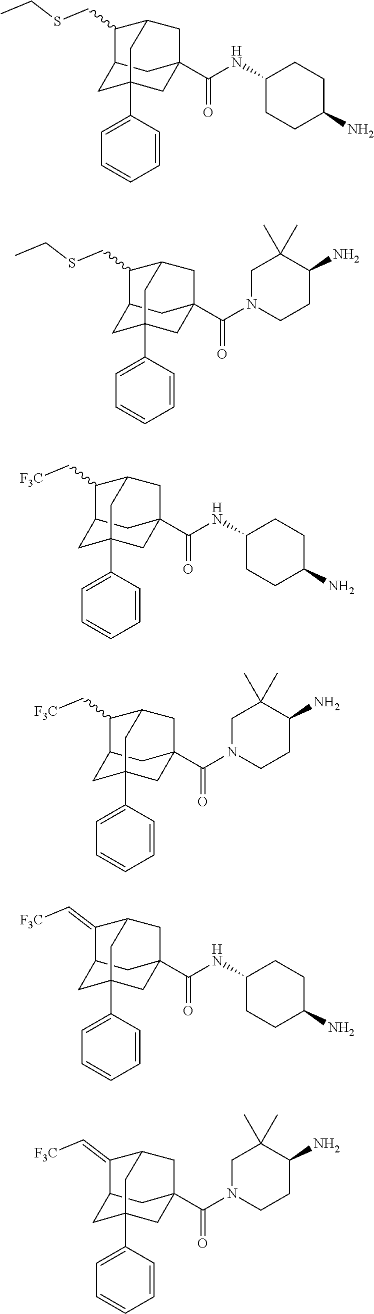 [SMILES: CCSCC1C2CC3(C(=O)N[C@H]4CC[C@H](N)CC4)CC1CC(c1ccccc1)(C2)C3 ; CCSCC1C2CC3(C(=O)N4CC[C@H](N)C(C)(C)C4)CC1CC(c1ccccc1)(C2)C3 ; N[C@H]1CC[C@H](NC(=O)C23CC4CC(c5ccccc5)(CC(C2)C4CC(F)(F)F)C3)CC1 ; CC1(C)CN(C(=O)C23CC4CC(c5ccccc5)(CC(C2)C4CC(F)(F)F)C3)CC[C@@H]1N ; N[C@H]1CC[C@H](NC(=O)C23CC4CC(c5ccccc5)(CC(C2)C4=CC(F)(F)F)C3)CC1 ; CC1(C)CN(C(=O)C23CC4CC(c5ccccc5)(CC(C2)C4=CC(F)(F)F)C3)CC[C@@H]1N]

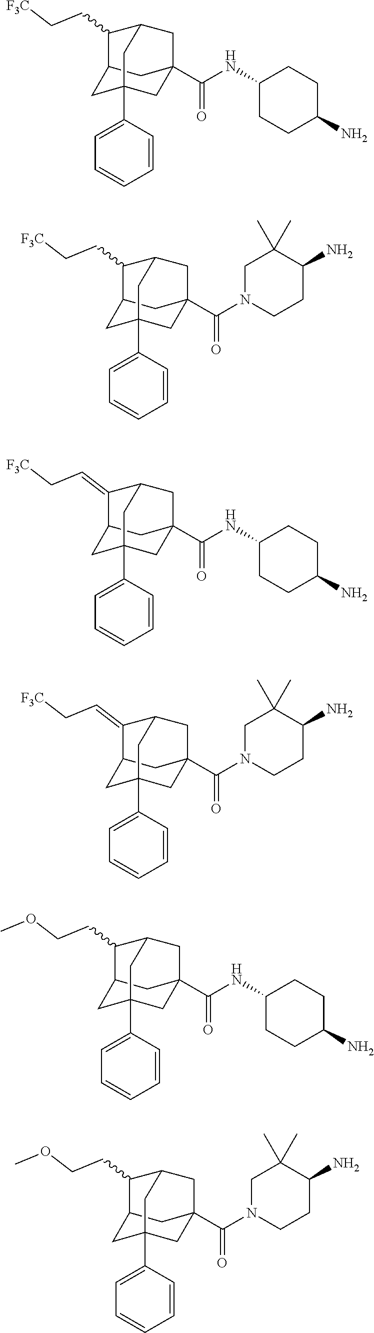 [SMILES: N[C@H]1CC[C@H](NC(=O)C23CC4CC(c5ccccc5)(CC(C2)C4CCC(F)(F)F)C3)CC1 ; CC1(C)CN(C(=O)C23CC4CC(c5ccccc5)(CC(C2)C4CCC(F)(F)F)C3)CC[C@@H]1N ; N[C@H]1CC[C@H](NC(=O)C23CC4CC(c5ccccc5)(CC(C2)C4=CCC(F)(F)F)C3)CC1 ; CC1(C)CN(C(=O)C23CC4CC(c5ccccc5)(CC(C2)C4=CCC(F)(F)F)C3)CC[C@@H]1N ; COCCC1C2CC3(C(=O)N[C@H]4CC[C@H](N)CC4)CC1CC(c1ccccc1)(C2)C3 ; COCCC1C2CC3(C(=O)N4CC[C@H](N)C(C)(C)C4)CC1CC(c1ccccc1)(C2)C3]

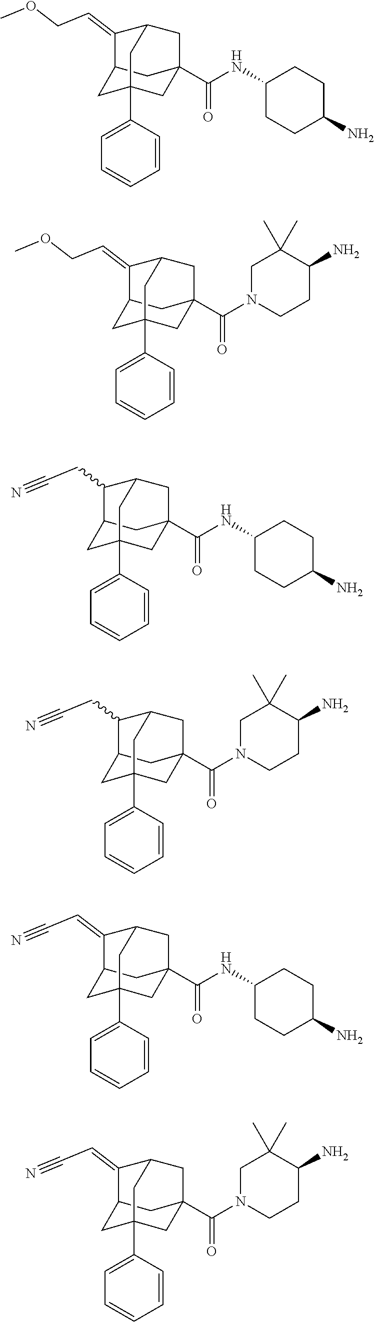 [SMILES: COCC=C1C2CC3(C(=O)N[C@H]4CC[C@H](N)CC4)CC1CC(c1ccccc1)(C2)C3 ; COCC=C1C2CC3(C(=O)N4CC[C@H](N)C(C)(C)C4)CC1CC(c1ccccc1)(C2)C3 ; N#CCC1C2CC3(C(=O)N[C@H]4CC[C@H](N)CC4)CC1CC(c1ccccc1)(C2)C3 ; CC1(C)CN(C(=O)C23CC4CC(c5ccccc5)(CC(C2)C4CC#N)C3)CC[C@@H]1N ; N#CC=C1C2CC3(C(=O)N[C@H]4CC[C@H](N)CC4)CC1CC(c1ccccc1)(C2)C3 ; CC1(C)CN(C(=O)C23CC4CC(c5ccccc5)(CC(C2)C4=CC#N)C3)CC[C@@H]1N]

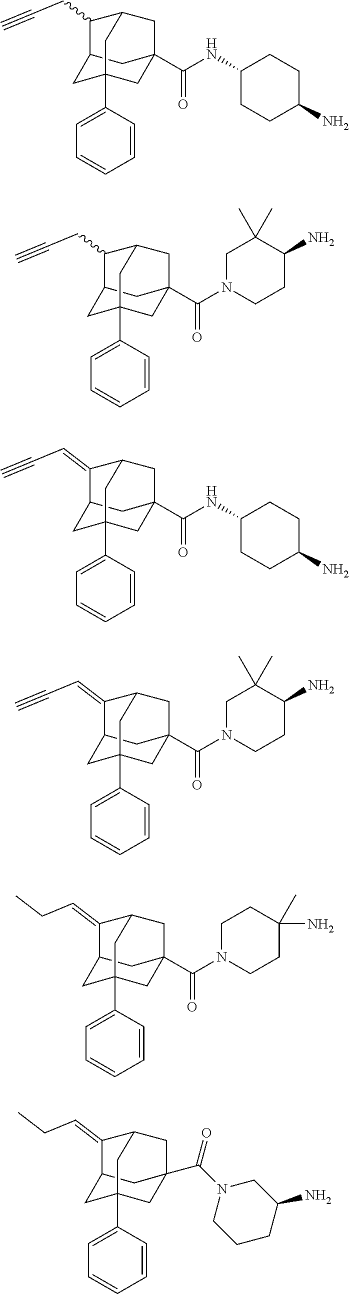 [SMILES: C#CCC1C2CC3(C(=O)N[C@H]4CC[C@H](N)CC4)CC1CC(c1ccccc1)(C2)C3 ; C#CCC1C2CC3(C(=O)N4CC[C@H](N)C(C)(C)C4)CC1CC(c1ccccc1)(C2)C3 ; C#CC=C1C2CC3(C(=O)N[C@H]4CC[C@H](N)CC4)CC1CC(c1ccccc1)(C2)C3 ; C#CC=C1C2CC3(C(=O)N4CC[C@H](N)C(C)(C)C4)CC1CC(c1ccccc1)(C2)C3 ; CCC=C1C2CC3(C(=O)N4CCC(C)(N)CC4)CC1CC(c1ccccc1)(C2)C3 ; CCC=C1C2CC3(C(=O)N4CCC[C@H](N)C4)CC1CC(c1ccccc1)(C2)C3]

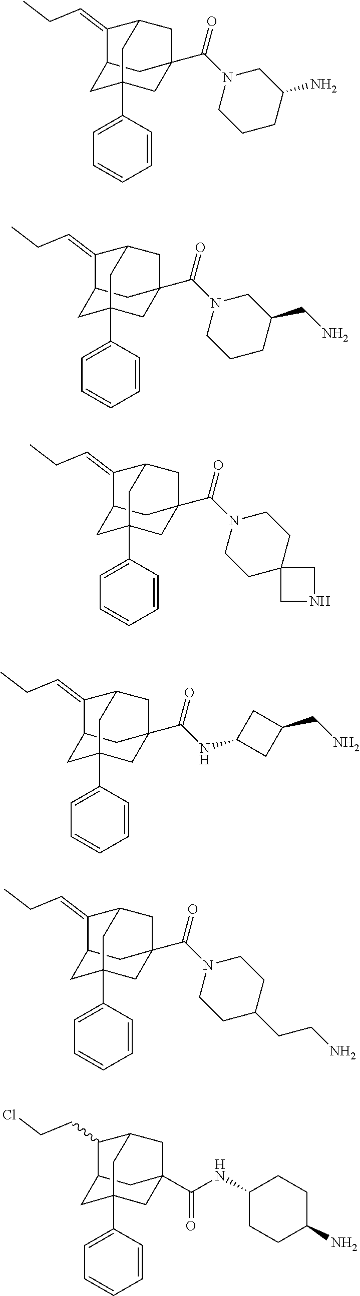 [SMILES: CCC=C1C2CC3(C(=O)N4CCC[C@@H](N)C4)CC1CC(c1ccccc1)(C2)C3 ; CCC=C1C2CC3(C(=O)N4CCC[C@H](CN)C4)CC1CC(c1ccccc1)(C2)C3 ; CCC=C1C2CC3(C(=O)N4CCC5(CC4)CNC5)CC1CC(c1ccccc1)(C2)C3 ; CCC=C1C2CC3(C(=O)N[C@H]4C[C@H](CN)C4)CC1CC(c1ccccc1)(C2)C3 ; CCC=C1C2CC3(C(=O)N4CCC(CCN)CC4)CC1CC(c1ccccc1)(C2)C3 ; N[C@H]1CC[C@H](NC(=O)C23CC4CC(c5ccccc5)(CC(C2)C4CCCl)C3)CC1]

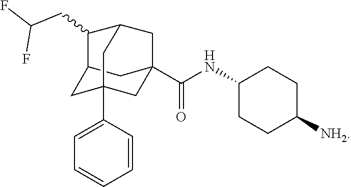 [SMILES: N[C@H]1CC[C@H](NC(=O)C23CC4CC(c5ccccc5)(CC(C2)C4CC(F)F)C3)CC1]

21. The method of claim 20, wherein the compound is selected from the group consisting of:

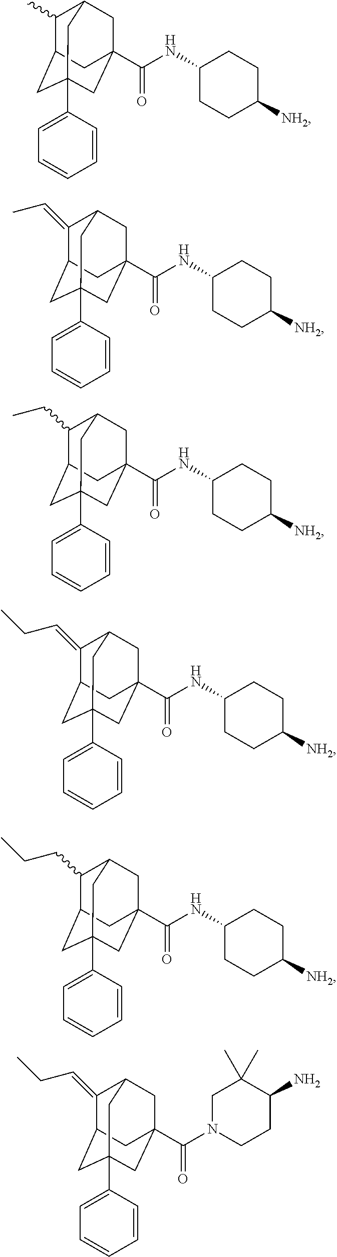

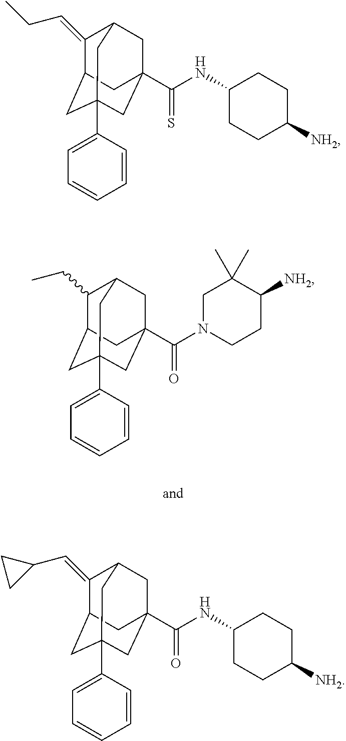 [SMILES: CCC=C1C2CC3(C(=S)N[C@H]4CC[C@H](N)CC4)CC1CC(c1ccccc1)(C2)C3 ; CCC1C2CC3(C(=O)N4CC[C@H](N)C(C)(C)C4)CC1CC(c1ccccc1)(C2)C3 ; N[C@H]1CC[C@H](NC(=O)C23CC4CC(c5ccccc5)(CC(C2)C4=CC2CC2)C3)CC1]

22. The method of claim 20, including administering a therapeutic amount of a therapeutic agent selected from the group consisting of Ribavirin, viral RNA-dependent-RNA polymerase inhibitors including favipiravir, Triazavirin, Remdesivir (GS-5734), monoclonal antibody therapies including, ZMapp, REGN3470-3471-3479, mAb 114, vaccines including, cAd3-EBOZ, rVSV-ZEBOV, small interfering RNAs and microRNAs and immunomodulators.

23. A compound represented by Structural Formula 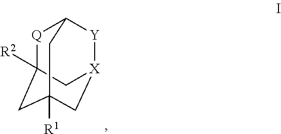
or a pharmaceutically acceptable salt, and a pharmaceutically acceptable carrier, diluent, or vehicle thereof, wherein:
X is 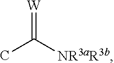
Y is CH₂, and Q is CH₂ or CR²³R²⁴, or
X is CH, Y is 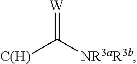
and Q is CH₂;
W is selected from the group consisting of O and S;
and when X is 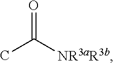
Y is CH₂, and Q is CH₂, then
R¹ is selected from (C₆ to C₁₀) aryl and (C₂ to C₉) heteroaryl, wherein
each of the said (C₆ to C₁₀) aryl and (C₂ to C₉) heteroaryl is optionally substituted with at least one R⁸ group;
R² is selected from the group consisting of 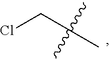 [SMILES: *CCl]
Br, and 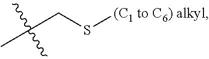
and
NR^{3a}R^{3b} is selected from the group consisting of 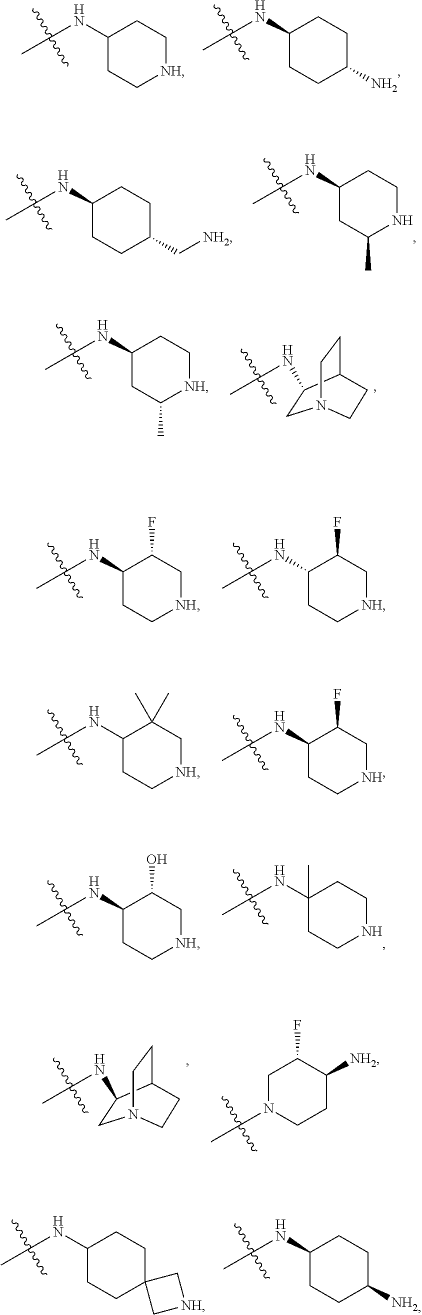 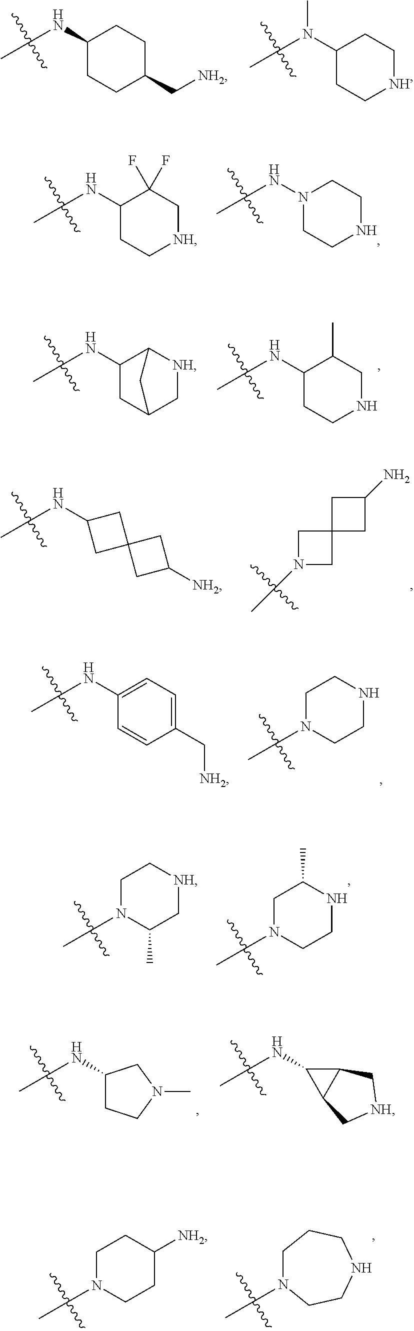 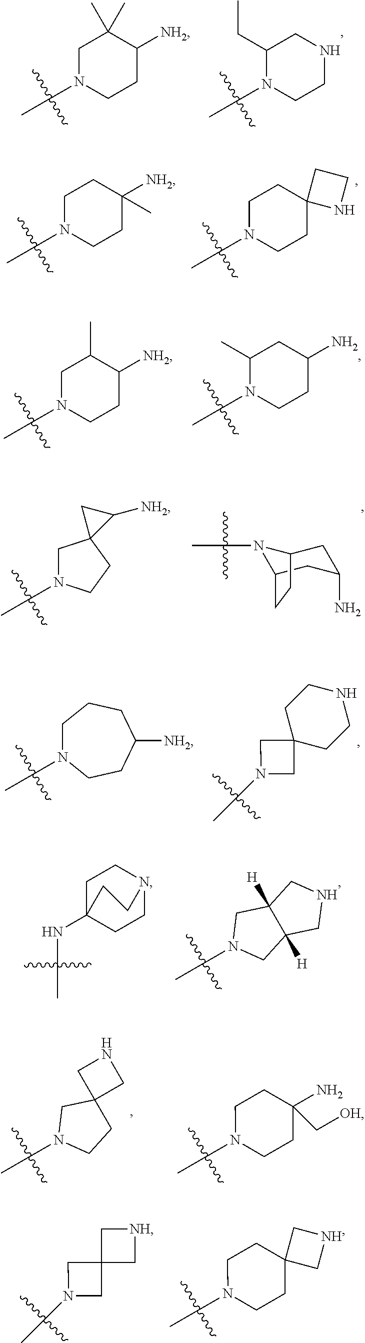 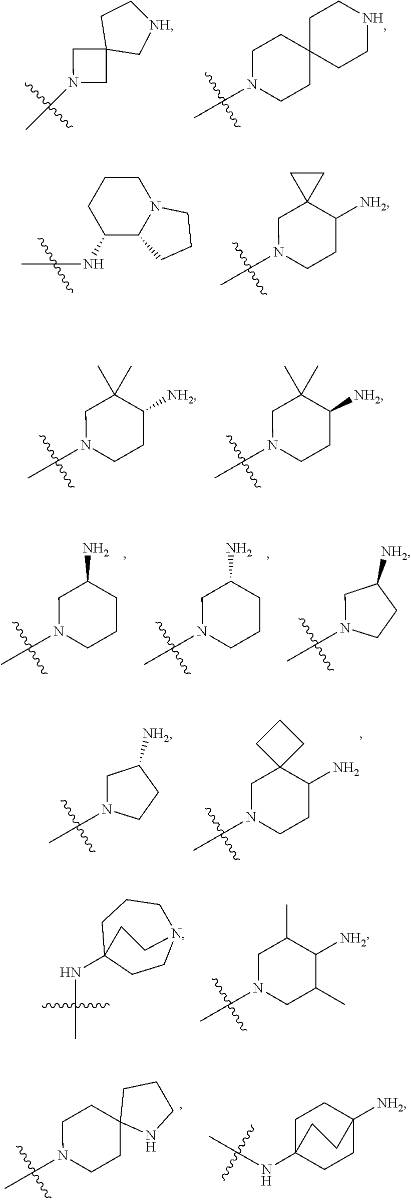 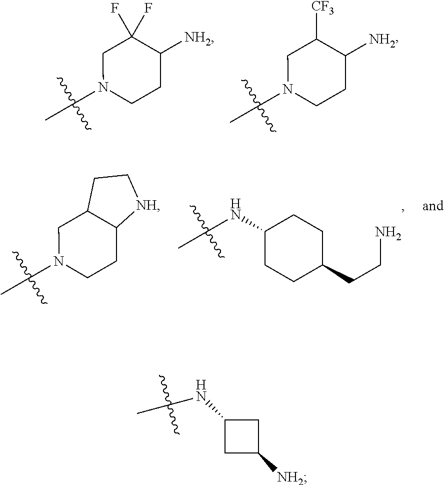 [SMILES: *N1CCC(N)C(F)(F)C1 ; *N1CCC(N)C(C(F)(F)F)C1 ; *N1CCC2NCCC2C1 ; *N[C@H]1CC[C@H](CCN)CC1 ; *N[C@H]1C[C@H](N)C1]
and when X is CH, Y is 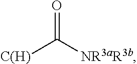
and Q is CH₂, then
R¹ is selected from (C₆ to C₁₀) aryl and (C₂ to C₉) heteroaryl, wherein
each of the said (C to C₁₀) aryl and (C₂ to C₉) heteroaryl is optionally substituted with at least one R⁸ group;
R² is selected from the group consisting of halogen, methyl, ethyl, propyl, chloromethyl, chloroethyl, methylthiomethyl, and thiomethyl;
NR^{3a}R^{3b} is selected from the group consisting of 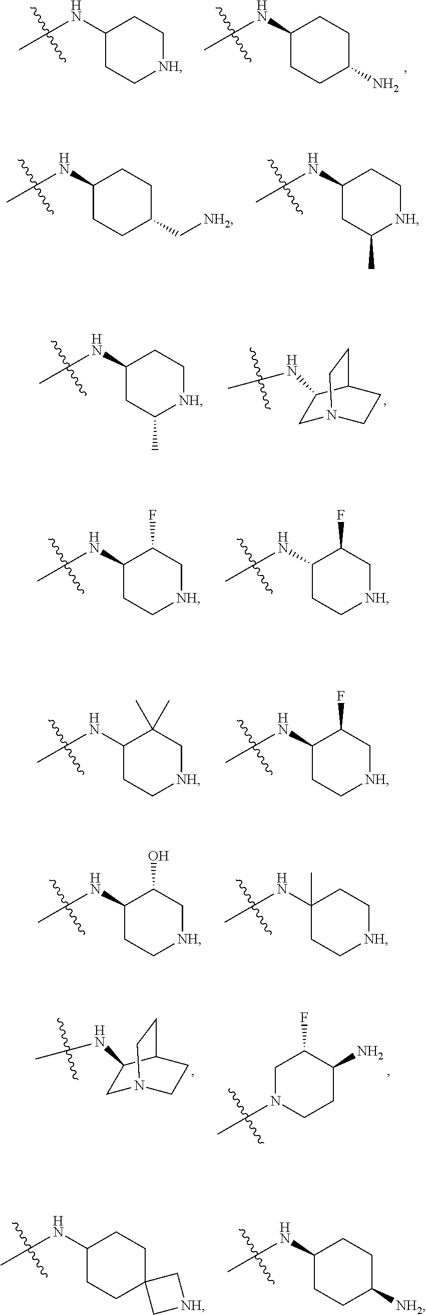 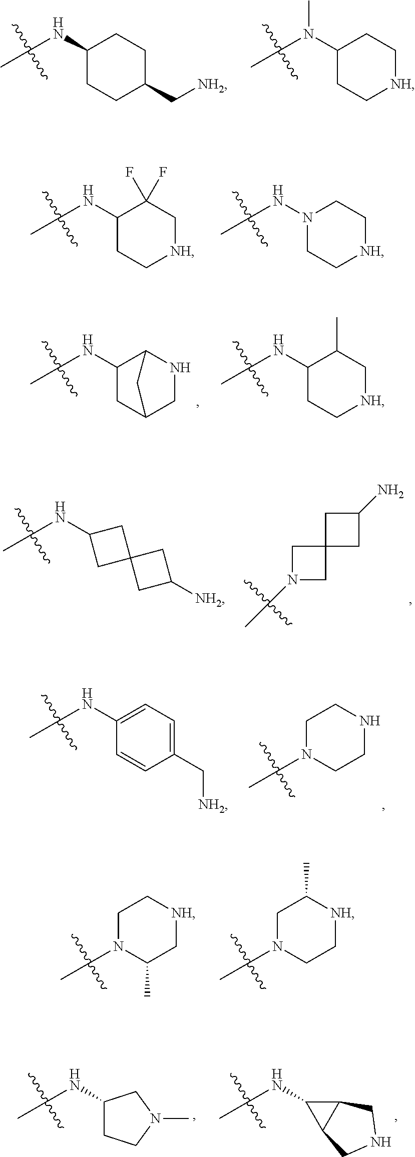 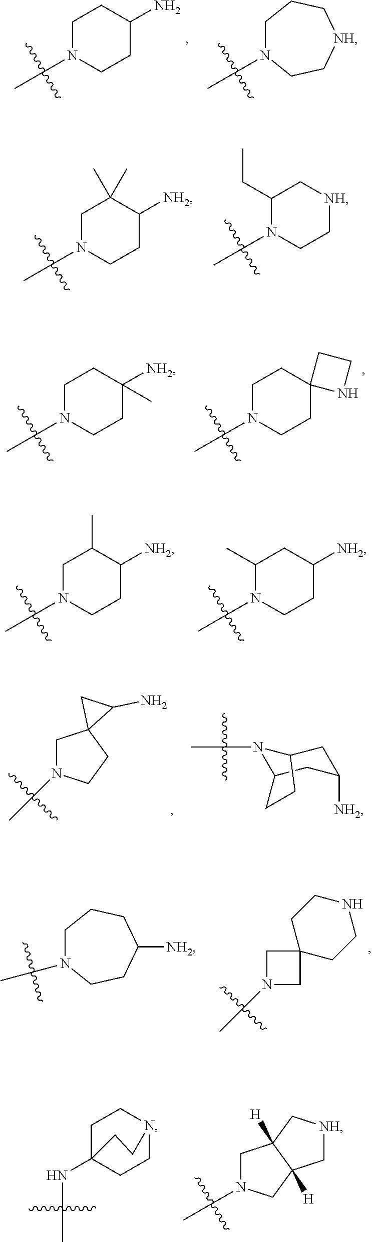 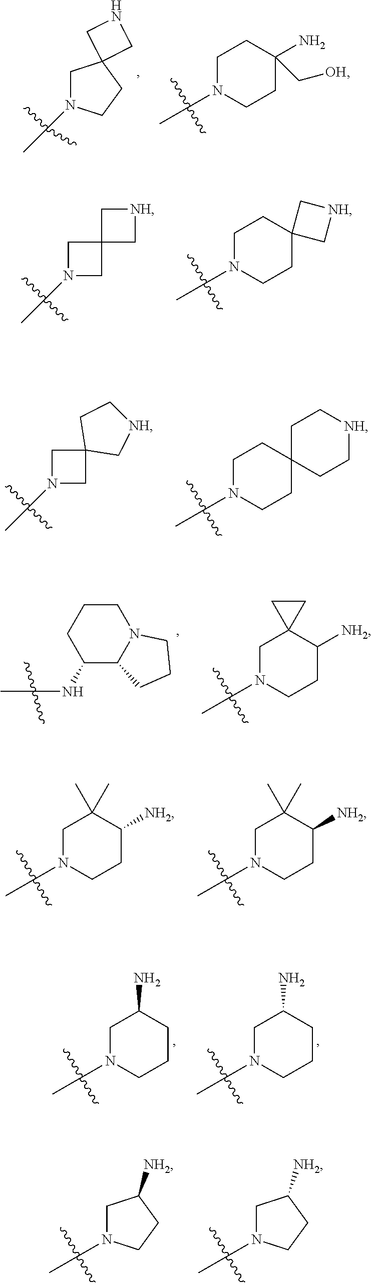 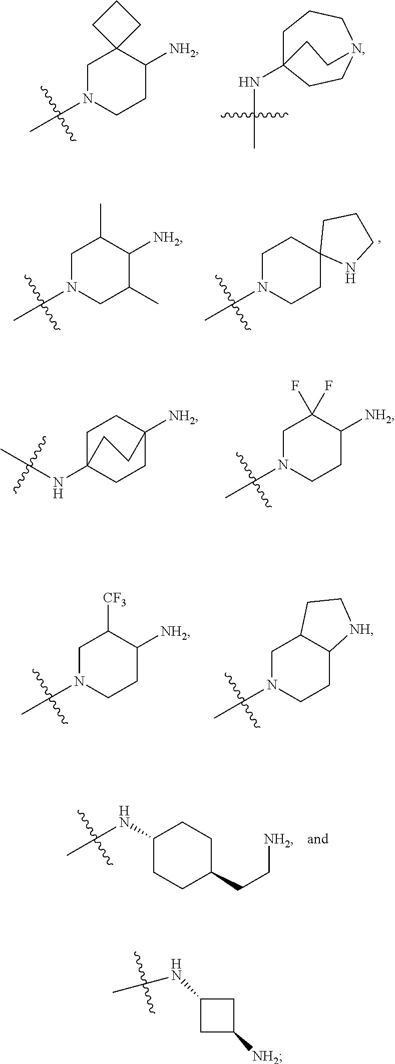
and when X is 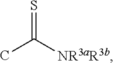
Y is CH₂, and Q is CH₂; or
X is CH, Y is 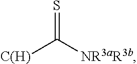
and Q is CH₂; or
X is 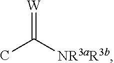
Y is CH₂, W is O or S, and Q is CR²³R²⁴ then
R¹ is selected from (C₆ to C₁₀) aryl and (C₂ to C₉) heteroaryl, wherein
each of the said (C₆ to C₁₀) aryl and (C₂ to C₉) heteroaryl is optionally substituted with at least one R⁸ group;
and when Q is CH₂, then
R² is selected from hydrogen, halogen, OH, nitro, CF₃, —NR^{6a}R^{6b}, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, —C(O)R⁷, —C(O)NR^{6a}R^{6b}, —S(O)ₘR⁷, —S(O)ₘNR^{6a}R^{6b}, —NR^{6a}S(O)ₘR⁷, —(CH₂)ₙC(O)OR⁷, —(CH₂)ₙC(O)N(R^{6a}R^{6b}), —(CH₂)ₙN(R^{6a}R^{6b}), —OC(O)R⁷, —NR^{6a}C(O)R⁷, and —NR^{6a}C(O)N(R^{6a}R^{6b}), wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁸ group;
and when Q is CR²³R²⁴ then R² is hydrogen;
NR^{3a}R^{3b} is selected from the group consisting of 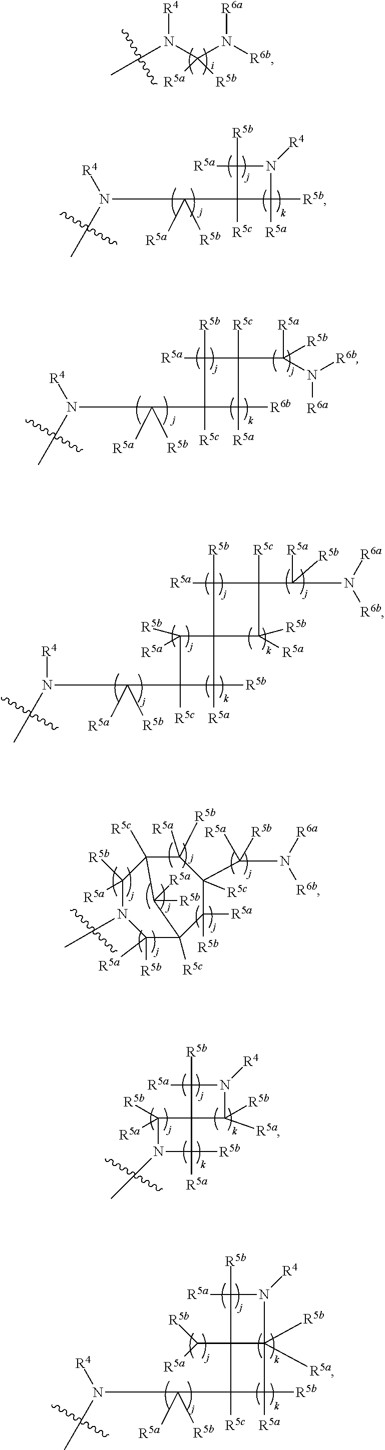 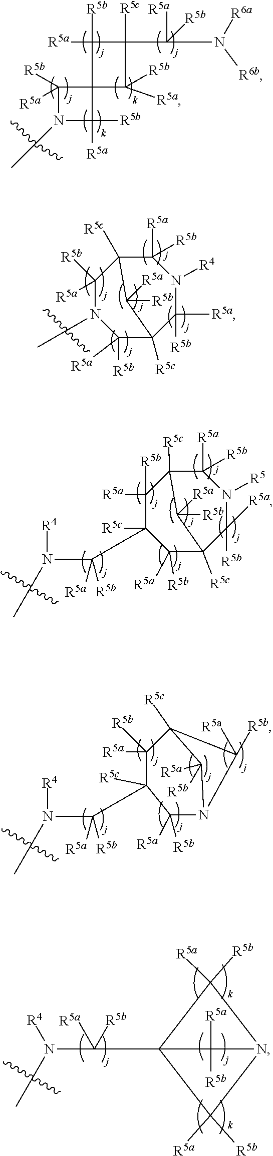 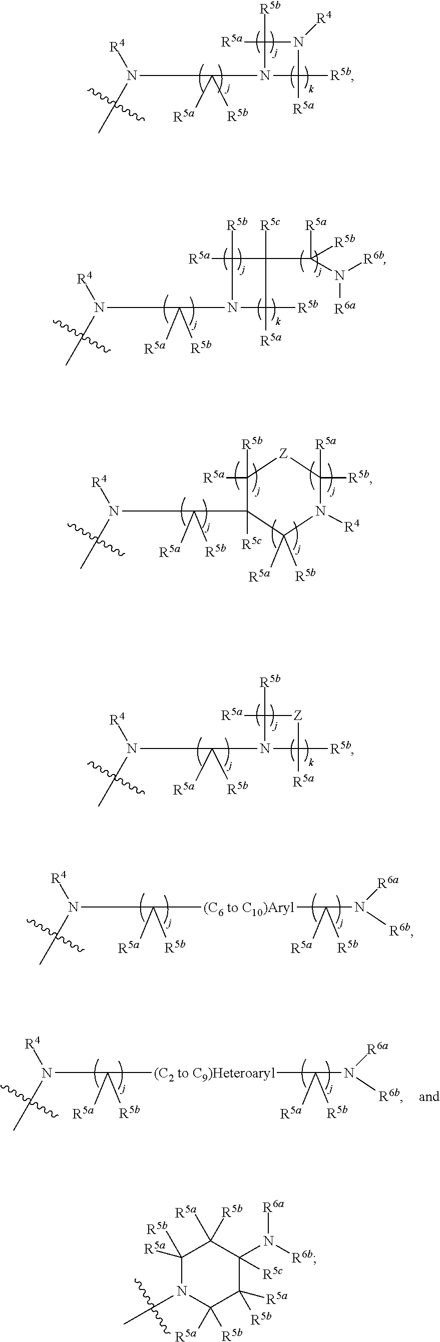
Z is selected from the group consisting of —O—, —S—, —S(O)—, and —S(O)₂—;
each R⁴ is independently selected from hydrogen, (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C to C₁₀) cycloalkenyl, (C₂ to C₉) cycloheteroalkyl, (C₆ to C₁₀) aryl, (C₂ to C₉) heteroaryl, (C₆ to C₁₀) arylene, and (C₂ to C₉) heteroarylene, wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C to C₁₀) cycloalkenyl, (C₂ to C₉) cycloheteroalkyl, (C₆ to C₁₀) aryl, (C₂ to C₉) heteroaryl, (C₆ to C₁₀) arylene, and (C₂ to C₉) heteroarylene is optionally substituted with at least one R⁸ group;
each of the R^{5a}, R^{5b}, and R^{5c} is independently selected from hydrogen, halogen, OH, nitro, CF₃, —NR^{6a}R^{6b}, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, —C(O)R⁷, —C(O)NR^{6a}R^{6b}, —S(O)ₘR⁷, —S(O)ₘNR^{6a}R^{6b}, —NR^{6a}S(O)ₘR⁷, —(CH₂)ₙC(O)OR⁷, —(CH₂)ₙC(O)N(R^{6a}R^{6b}), —(CH₂)ₙN(R^{6a}R^{6b}), —OC(O)R⁷, —NR^{6a}C(O)R⁷, and —NR^{6a}C(O)N(R^{6a}R^{6b}), wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁸ group;
or R^{5a} and R^{5b} may be taken together with the carbon atom to which they are attached to form a (C₃ to C₁₀) cycloalkyl ring, wherein
the said (C₃ to C₁₀) cycloalkyl ring is optionally substituted with at least one R⁸ group;
each of the R^{6a} and R^{6b} is independently selected from hydrogen, (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, (C₆ to C₁₀) arylene, and (C₂ to C₉) heteroarylene, wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, (C₆ to C₁₀) arylene, and (C₂ to C₉) heteroarylene is optionally substituted with at least one R⁸ group,
or R^{6a} and R^{6b} may be taken together with the nitrogen atom to which they are attached to form a (C₂ to C₁₀) cycloheteroalkyl ring, wherein
said (C₂ to C₁₀) cycloheteroalkyl ring has 1 to 3 ring heteroatoms selected from the group consisting of N, O, and S, and wherein
the said (C₂ to C₁₀) cycloheteroalkyl ring is optionally substituted with at least one R⁸ group;
each of the R⁷ is independently selected from hydrogen, (C₁ to C₁₀) alkyl, (C₂ to C₁₀) alkenyl, (C₂ to C₁₀) alkynyl, (C₃ to C₁₀) cycloalkyl, (C to C₁₀) cycloalkenyl, (C₂ to C₉) cycloheteroalkyl, (C₆ to C₁₀) aryl, and (C₂ to C₉) heteroaryl, wherein
each of the said (C₁ to C₁₀) alkyl, (C₂ to C₁₀) alkenyl, (C₂ to C₁₀) alkynyl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁸ group;
each R⁸ is independently selected from hydrogen, halogen, OH, nitro, CF₃, —NR^{9a}R^{9b}, oxo, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, (C₆ to C₁₀) arylene, (C₂ to C₉) heteroarylene, (C₃ to C₁₀) cycloalkylene, (C₂ to C₁₀) cycloheteroalkylene, —C(O)R¹⁰, —C(O)NR^{9a}R^{9b}, —S(O)ₘR¹⁰, —S(O)ₘNR^{9a}R^{9b}, —NR^{9a}S(O)ₘR¹⁰, —(CH₂)ₙC(O)OR¹⁰, —(CH₂)ₙC(O)N(R^{9a}R^{9b}), —(CH₂)ₙN(R^{9a}R^{9b}), —OC(O)R¹⁵, —O(CH₂)ₙO—, —NR^{9a}C(O)R¹⁰, and —NR^{9a}C(O)N(R^{9a}R^{9b}), wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, (C₂ to C₉) heteroarylene, (C₃ to C₁₀) cycloalkylene, and (C₂ to C₁₀) cycloheteroalkylene is optionally substituted with at least one R¹¹ group;
each of the R^{9a} and R^{9b} is independently selected from hydrogen, (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, (C₆ to C₁₀) arylene, and (C₂ to C₉) heteroarylene, wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, and (C₂ to C₉) heteroarylene is optionally substituted with at least one R¹¹ group,
or R^{9a} and R^{9b} may be taken together with the nitrogen atom to which they are attached to form a (C₂ to C₁₀) cycloheteroalkyl ring, wherein
said (C₂ to C₁₀) cycloheteroalkyl ring has 1 to 3 ring heteroatoms selected from the group consisting of N, O, and S, and wherein
the said (C₂ to C₁₀) cycloheteroalkyl ring is optionally substituted with at least one R¹¹ group;
each R¹⁰ is independently selected from hydrogen, (C₁ to C₁₀) alkyl, (C₂ to C₁₀) alkenyl, (C₂ to C₁₀) alkynyl, (C₃ to C₁₀) cycloalkyl, (C₅ to C₁₀) cycloalkenyl, (C₂ to C₁₀) cycloheteroalkyl, (C₆ to C₁₀) aryl, and (C₂ to C₉) heteroaryl, wherein
each of the said (C₁ to C₁₀) alkyl, (C₂ to C₁₀) alkenyl, (C₂ to C₁₀) alkynyl, (C₃ to C₁₀) cycloalkyl, (C₅ to C₁₀) cycloalkenyl, (C₂ to C₁₀) cycloheteroalkyl, (C₆ to C₁₀) aryl, and (C₂ to C₉) heteroaryl is optionally substituted with at least one R¹¹ group;
each R¹¹ is independently selected from hydrogen, halogen, OH, nitro, CF₃, —NR^{12a}R^{12b}, oxo, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, (C₂ to C₉) heteroarylene, (C₃ to C₁₀) cycloalkylene, (C₂ to C₁₀) cycloheteroalkylene, —C(O)R¹⁸, —C(O)NR^{12a}R^{12b}, —S(O)ₘR¹³, —S(O)ₘNR^{12a}R^{12b}, —NR^{12a}S(O)ₘR¹³, —(CH₂)ₙC(O)OR¹³, —(CH₂)ₙC(O)N(R^{12a}R^{12b}), —(CH₂)ₙN(R^{12a}R^{12b}), —OC(O)R¹³, —NR^{12a}C(O)R¹³, and —NR^{12a}C(O)N(R^{12a}R^{12b}) wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₂ to C₉) heteroaryl, (C₆ to C₁₀) aryl, (C₆ to C₁₀) arylene, (C₂ to C₉) heteroarylene, (C₃ to C₁₀) cycloalkylene, and (C₂ to C₁₀) cycloheteroalkylene is optionally substituted with at least one R¹⁴ group;
each of the R^{12a} and R^{12b} is independently selected from hydrogen, (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, and (C₂ to C₉) heteroarylene, wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, and (C₂ to C₉) heteroarylene is optionally substituted with at least one R¹⁴ group,
or R^{12a} and R^{12b} may be taken together with the nitrogen atom to which they are attached to form a (C₂ to C₁₀) cycloheteroalkyl ring, wherein
said (C₂ to C₁₀) cycloheteroalkyl ring has 1 to 3 ring heteroatoms selected from the group consisting of N, O, and S, and wherein
the said (C₂ to C₁₀) cycloheteroalkyl ring is optionally substituted with at least one R¹⁴ group;
each R¹³ is independently selected from hydrogen, halogen, OH, nitro, CF₃, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₁₀) cycloheteroalkyl, (C₆ to C₁₀) aryl, and (C₂ to C₉) heteroaryl, wherein
each of the said (C₁ to C₁₀) alkyl, (C₂ to C₁₀) alkenyl, (C₂ to C₁₀) alkynyl, (C₃ to C₁₀) cycloalkyl, (C₅ to C₁₀) cycloalkenyl, (C₂ to C₁₀) cycloheteroalkyl, (C₂ to C₉) heteroaryl, and (C₆ to C₁₀) aryl is optionally substituted with at least one R¹⁴ group;
each R¹⁴ is independently selected from hydrogen, halogen, OH, nitro, CF₃, —NR^{15a}R^{15b}, oxo, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, (C₂ to C₉) heteroarylene, (C₃ to C₁₀) cycloalkylene, (C₂ to C₁₀) cycloheteroalkylene, —C(O)R¹⁶, —C(O)NR^{15a}R^{15b}, —S(O)ₘR¹⁶, —S(O)ₘNR^{15a}R^{15b}, —NR^{15a}S(O)ₘR¹⁶, —(CH₂)ₙC(O)OR¹⁶, —(CH₂)ₙC(O)N(R^{15a}R^{15b}), —(CH₂)ₙN(R^{15a}R^{15b}), —OC(O)R¹⁶, —NR^{15a}C(O)R¹⁶, and —NR^{15a}C(O)N(R^{15a}R^{15b}) wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, (C₂ to C₉) heteroarylene, (C₃ to C₁₀) cycloalkylene, and (C₂ to C₁₀) cycloheteroalkylene is optionally substituted with at least one R¹⁷ group;
each of the R^{15a} and R^{15b} is independently selected from hydrogen, (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, and (C₂ to C₉) heteroaryl, wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, and (C₂ to C₉) heteroaryl is optionally substituted with at least one R¹⁷ group,
or R^{15a} and R^{15b} may be taken together with the nitrogen atom to which they are attached to form a (C₂ to C₁₀) cycloheteroalkyl ring, wherein
said (C₂ to C₁₀) cycloheteroalkyl ring has 1 to 3 ring heteroatoms selected from the group consisting of N, O, and S, and wherein
the said (C₂ to C₁₀) cycloheteroalkyl ring is optionally substituted with at least one R¹⁷ group;
each R¹⁶ is independently selected from hydrogen, halogen, OH, nitro, CF₃, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₁₀) cycloheteroalkyl, (C₆ to C₁₀) aryl, and (C₂ to C₉) heteroaryl;
each R¹⁷ is independently selected from hydrogen, halogen, OH, nitro, CF₃, —NR^{18a}R^{18b}, oxo, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₁₀) cycloheteroalkyl, (C to C₁₀) aryl, (C₂ to C₉) heteroaryl, (C₆ to C₁₀) arylene, (C₂ to C₉) heteroarylene, (C₃ to C₁₀) cycloalkylene, (C₂ to C₁₀) cycloheteroalkylene, —C(O)R¹⁹, —C(O)NR^{18a}R^{18b}, —S(O)ₘR¹⁹, —S(O)ₘNR^{18a}R^{18b}, —NR^{18a}S(O)ₘR¹⁹, —(CH₂)ₙC(O)OR¹⁹, —(CH₂)ₙC(O)N(R^{18a}R^{18b}), —(CH₂)ₙN(R^{18a}R^{18b}), —OC(O)R¹⁹, —NR^{18a}C(O)R¹⁹, and —NR^{18a}C(O)N(R^{18a}R^{18b}) wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, (C₂ to C₉) heteroarylene, (C₃ to C₁₀) cycloalkylene, and (C₂ to C₁₀) cycloheteroalkylene is optionally substituted with at least one R²⁰ group;
each of the R^{18a} and R^{18b} is independently selected from hydrogen, (C₁ to C₁₀) alkyl, (C₂ to C₁₀) alkenyl, (C₂ to C₁₀) alkynyl, (C₃ to C₁₀) cycloalkyl, (C₅ to C₁₀) cycloalkenyl, (C₂ to C₁₀) cycloheteroalkyl, (C₂ to C₉) heteroaryl, and (C₆ to C₁₀) aryl;
each R¹⁹ is independently selected from hydrogen, halogen, OH, nitro, CF₃, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl;
each R²⁰ is independently selected from hydrogen, halogen, OH, nitro, CF₃, —NR^{21a}R^{21b}, oxo, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C₆ to C₁₀) arylene, (C₂ to C₉) heteroarylene, (C₃ to C₁₀) cycloalkylene, (C₂ to C₁₀) cycloheteroalkylene, —C(O)R²², —C(O)NR^{21a}R^{21b}, —S(O)ₘR²², —S(O)ₘNR^{21a}R^{21b}, —NR^{21a}S(O)ₘR²², —(CH₂)ₙC(O)OR²², —(CH₂)ₙC(O)N(R^{21a}R^{21b}), —(CH₂)ₙN(R^{21a}R^{21b}), —OC(O)R²², —NR^{21a}C(O)R²², and —NR^{21a}C(O)N(R^{21a}R^{21b}) wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C₆ to C₁₀) aryl, (C₂ to C₉) heteroaryl, (C to C₁₀) arylene, (C₂ to C₉) heteroarylene, (C₃ to C₁₀) cycloalkylene, and (C₂ to C₁₀) cycloheteroalkylene is optionally substituted with at least one R²² group;
each of the R^{21a} and R^{21b} is independently selected from hydrogen, (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₁₀) cycloheteroalkyl, (C to C₁₀) aryl, and (C₂ to C₉) heteroaryl,
or R^{21a} and R^{21b} may be taken together with the nitrogen atom to which they are attached to form a (C₂ to C₁₀) cycloheteroalkyl ring, wherein
said (C₂ to C₁₀) cycloheteroalkyl ring has 1 to 3 ring heteroatoms selected from the group consisting of N, O, and S;
each R²² is independently selected from hydrogen, halogen, OH, nitro, CF₃, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cycloalkenyl, (C₂ to C₁₀) cycloheteroalkyl, (Ceto C₁₀) aryl, (C₂ to C₉) heteroaryl;
each R²³ is independently selected from hydrogen, halogen, OH, nitro, CF₃, —NR^{11a}R^{11b}, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cyclo-alkenyl, (C₂ to C₉) cycloheteroalkyl, (C₆ to C₁₀) aryl, (C₂ to C₉) heteroaryl, —C(O)R¹², —C(O)NR^{11a}R^{11b}, —S(O)ₘR¹², —S(O)ₘNR^{11a}R^{11b}, —NR^{11a}S(O)ₘR¹², —(CH₂)ₙC(O)OR¹², —(CH₂)ₙC(O)N(R^{11a}R^{11b}), —(CH₂)ₙN(R^{11a}R^{11b}), —OC(O)R¹², —NR^{11a}C(O)R¹², and —NR^{11a}C(O)N(R^{11a}R^{11b}) wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁸ group;
each R²⁴ is independently selected from halogen, OH, nitro, CF₃, —NR^{11a}R^{11b}, (C₁ to C₁₀) alkyl, (C₂ to C₁₀) alkenyl, (C₂ to C₁₀) alkynyl, (C₁ to C₁₀) alkoxy, aryloxy, cyano, (C₃ to C₁₀) cycloalkyl, (C₅ to C₁₀) cyclo-alkenyl, (C₂ to C₉) cycloheteroalkyl, (C₆ to C₁₀) aryl, (C₂ to C₉) heteroaryl, —C(O)R¹², —C(O)NR^{11a}R^{11b}, —S(O)ₘR¹², —S(O)ₘNR^{11a}R^{11b}, —NR^{11a}S(O)ₘR¹², —(CH₂)ₙC(O)OR¹², —(CH₂)ₙC(O)N(R^{11a}R^{11b}), —(CH₂)ₙN(R^{11a}R^{11b}), —OC(O)R¹², —NR^{11a}C(O)R¹², and —NR^{11a}C(O)N(R^{11a}R^{11b}), wherein
each of the said (C₁ to C₁₀) alkyl, (C₂ to C₁₀) alkenyl, (C₂ to C₁₀) alkynyl, (C₁ to C₁₀) alkoxy, aryloxy, (C₃ to C₁₀) cycloalkyl, (C₅ to C₁₀) cycloalkenyl, (C₂ to C₉) cycloheteroalkyl, (C₅ to C₁₀) aryl, and (C₂ to C₉) heteroaryl is optionally substituted with at least one R⁸ group;
or R²³ and R²⁴ may be taken together with the carbon atom to which they are attached to form a (C₃ to C₁₀) cycloalkyl or (C₂ to C₉) cycloheteroalkyl ring, wherein
the said (C₃ to C₁₀) cycloalkyl or (C₂ to C₉) cycloheteroalkyl ring is optionally substituted with at least one R⁸ group;
or R²³ and R²⁴ may be taken together with the carbon atom to which they are attached to form a carbonyl or alkene, which is optionally substituted with at least one R⁸ group;
i is 2, 3, 4, 5, or 6;
j is 0, 1, 2, 3, 4, or 5;
k is 1, 2, 3, 4, or 5;
m is 0, 1 or 2;
n is 0, 1, 2, 3, or 4;
with the proviso that when R¹ is phenyl, R² is hydrogen, X is 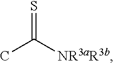
Y is CH₂, and Q is CH₂, then NR^{3a}R^{3b} is not 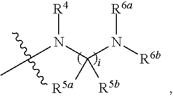
and with the proviso that the following compounds are excluded:

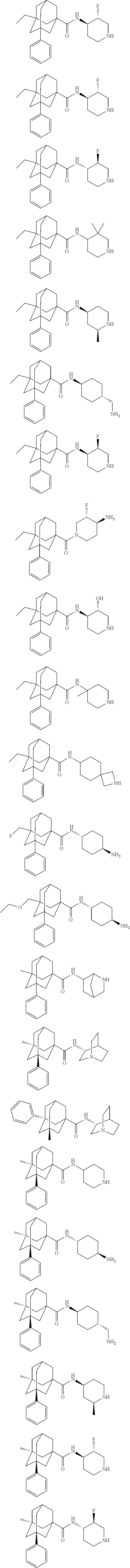

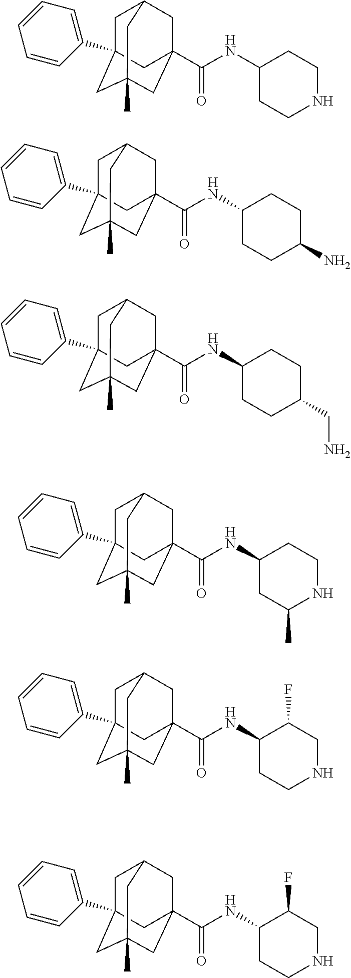

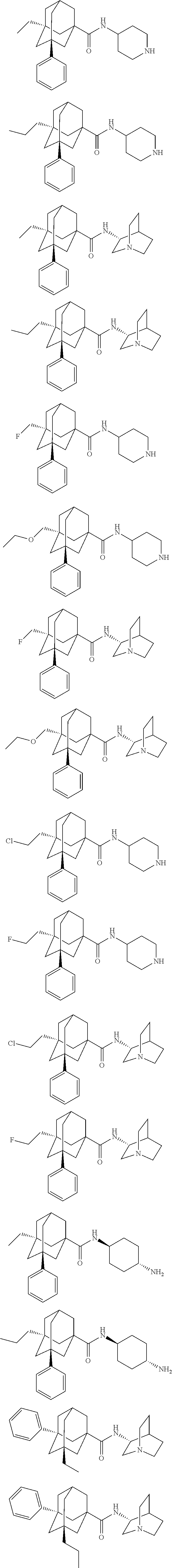

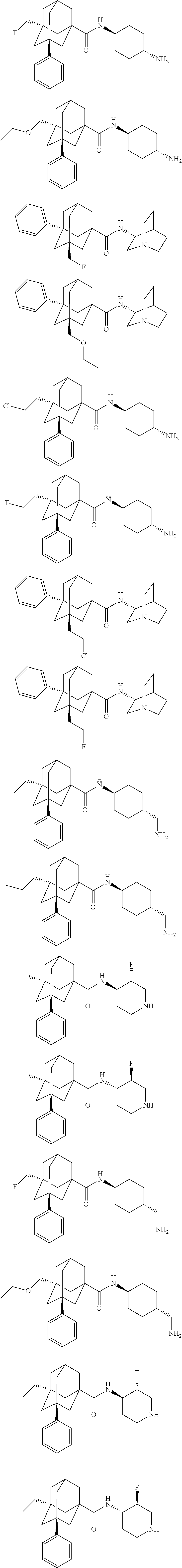

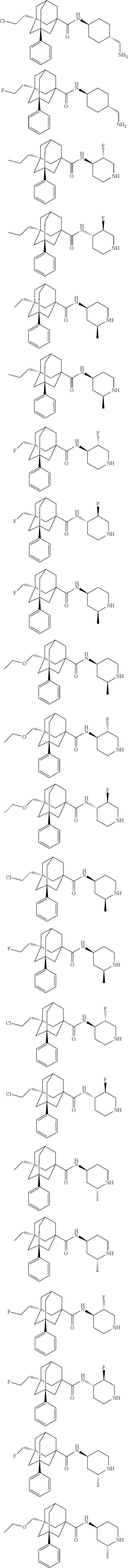

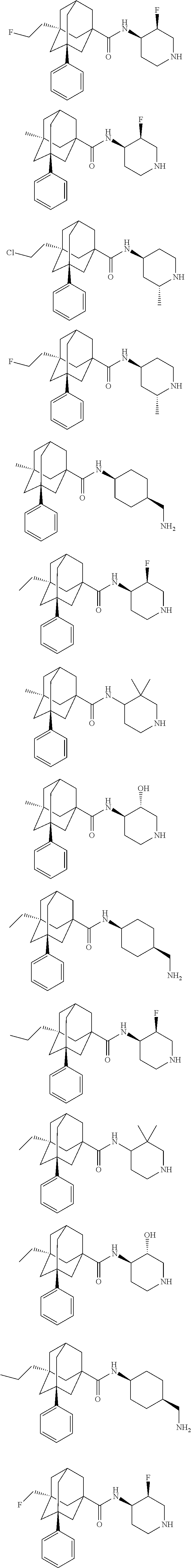

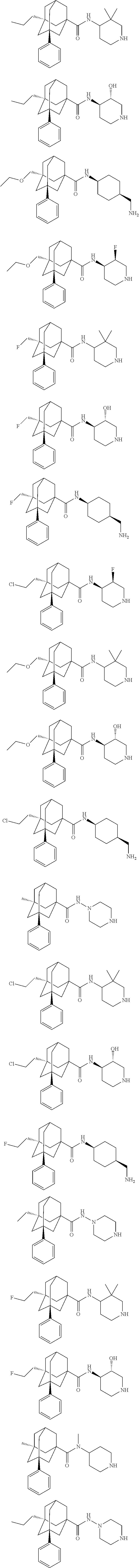

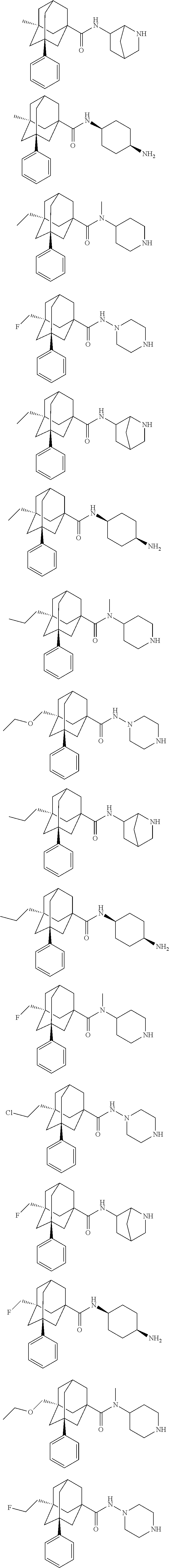

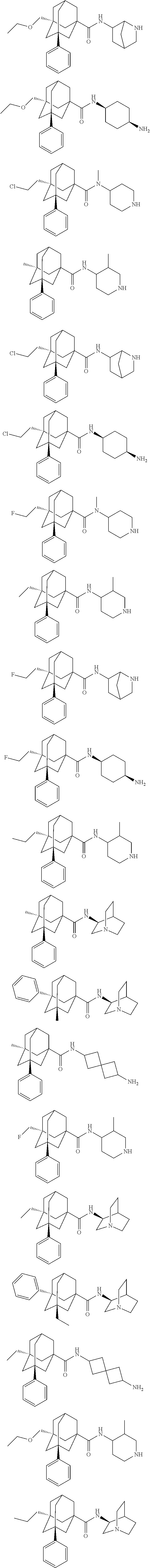

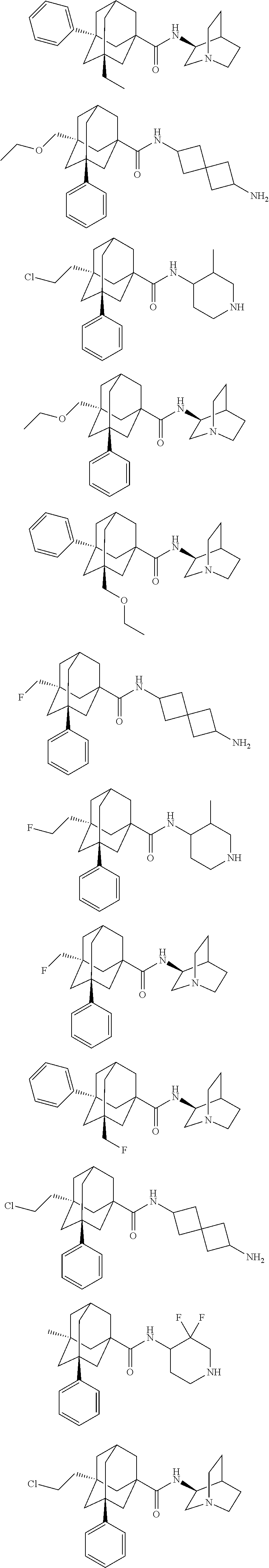

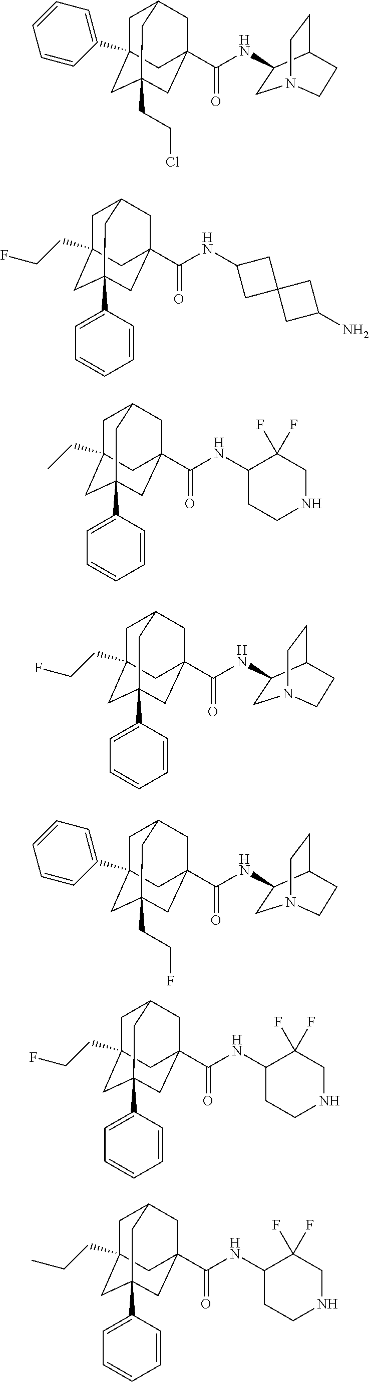

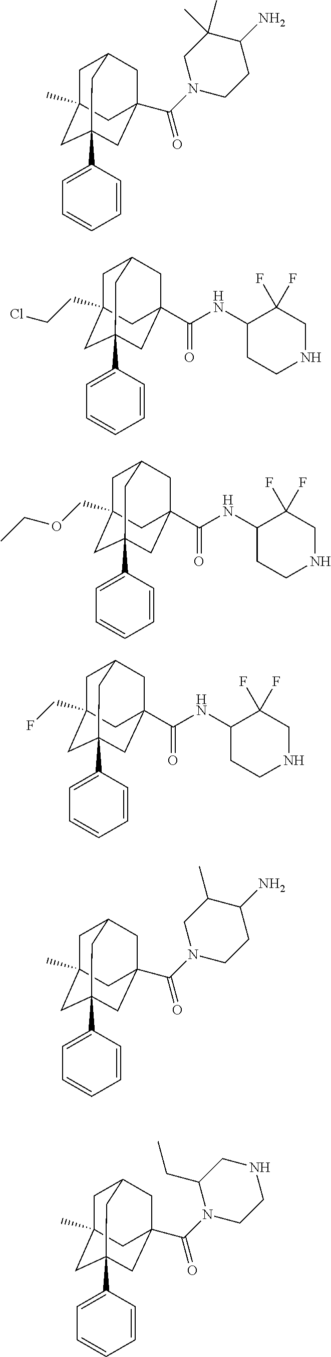

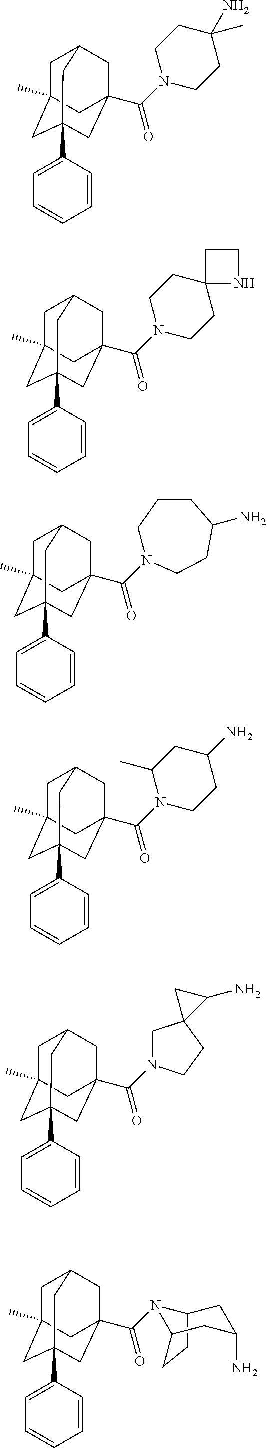

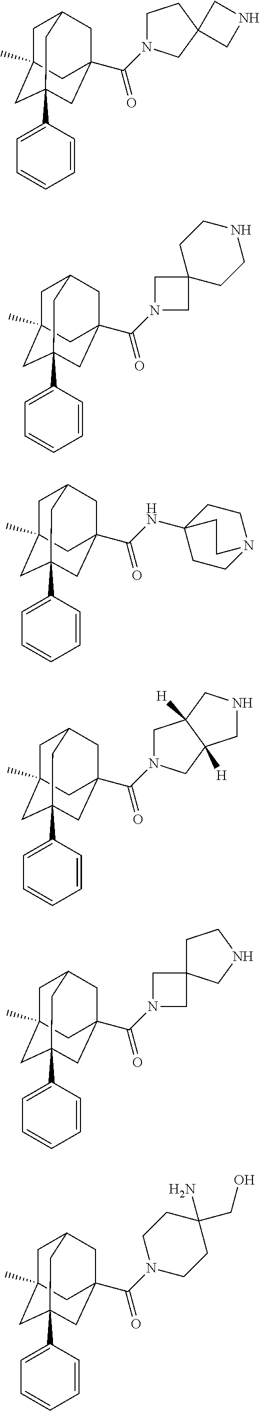

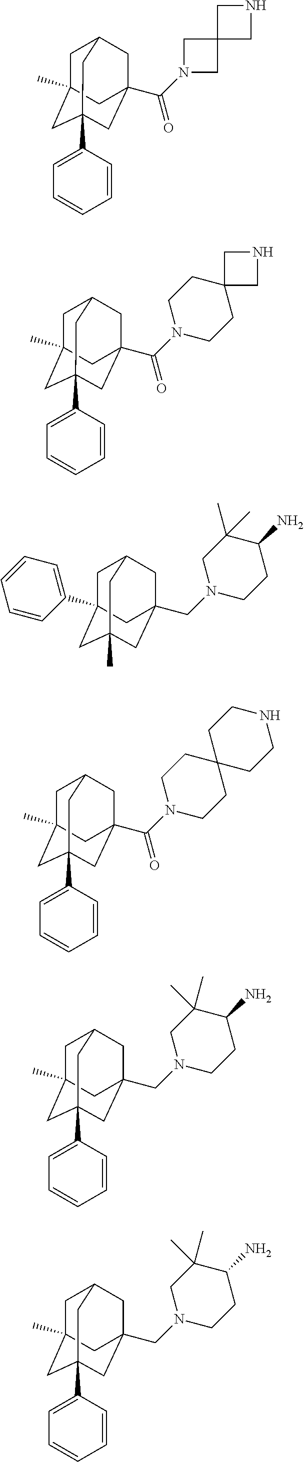 [SMILES: C[C@]12CC3CC(C(=O)N4CC5(CNC5)C4)(C1)C[C@@](c1ccccc1)(C3)C2 ; C[C@]12CC3CC(C(=O)N4CCC5(CC4)CNC5)(C1)C[C@@](c1ccccc1)(C3)C2 ; CC1(C)CN(CC23CC4C[C@](C)(C2)C[C@@](c2ccccc2)(C4)C3)CC[C@@H]1N ; C[C@]12CC3CC(C(=O)N4CCC5(CCNCC5)CC4)(C1)C[C@@](c1ccccc1)(C3)C2 ; CC1(C)CN(CC23CC4C[C@@](C)(C2)C[C@](c2ccccc2)(C4)C3)CC[C@@H]1N ; CC1(C)CN(CC23CC4C[C@@](C)(C2)C[C@](c2ccccc2)(C4)C3)CC[C@H]1N]

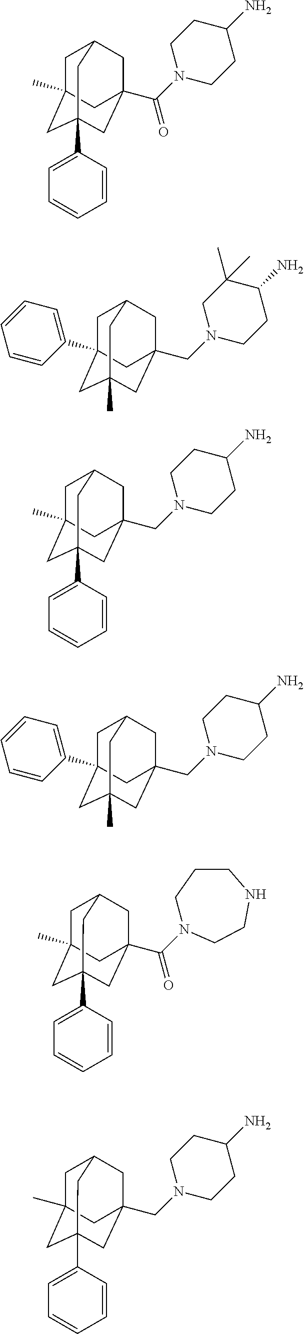 [SMILES: C[C@]12CC3CC(C(=O)N4CCC(N)CC4)(C1)C[C@@](c1ccccc1)(C3)C2 ; CC1(C)CN(CC23CC4C[C@](C)(C2)C[C@@](c2ccccc2)(C4)C3)CC[C@H]1N ; C[C@]12CC3CC(CN4CCC(N)CC4)(C1)C[C@@](c1ccccc1)(C3)C2 ; C[C@@]12CC3CC(CN4CCC(N)CC4)(C1)C[C@](c1ccccc1)(C3)C2 ; C[C@]12CC3CC(C(=O)N4CCCNCC4)(C1)C[C@@](c1ccccc1)(C3)C2 ; CC12CC3CC(CN4CCC(N)CC4)(C1)CC(c1ccccc1)(C3)C2]

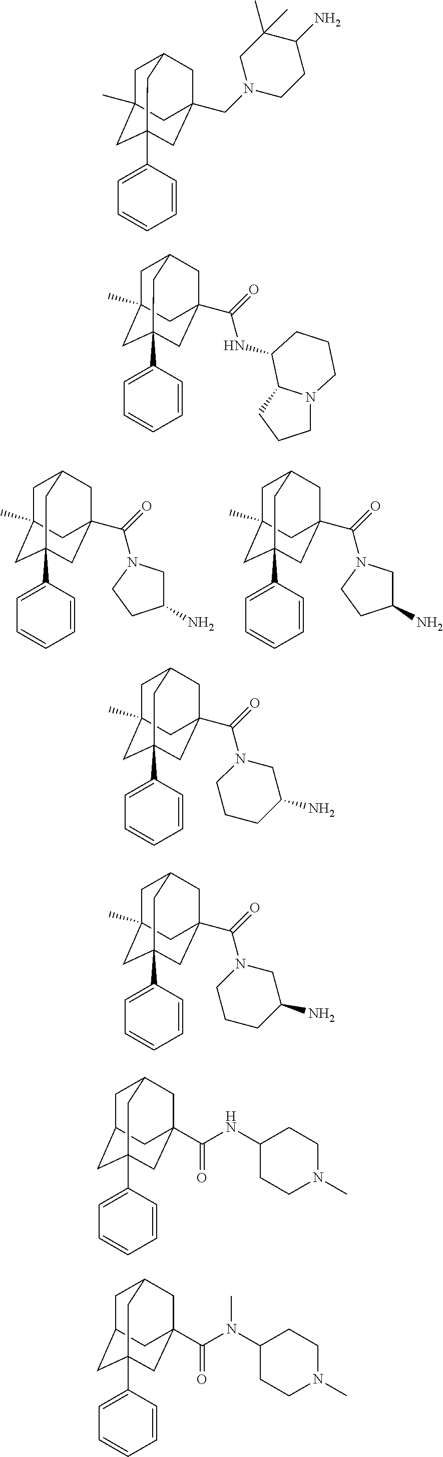

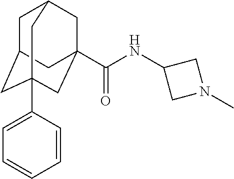 [SMILES: CN1CC(NC(=O)C23CC4CC(C2)CC(c2ccccc2)(C4)C3)C1]

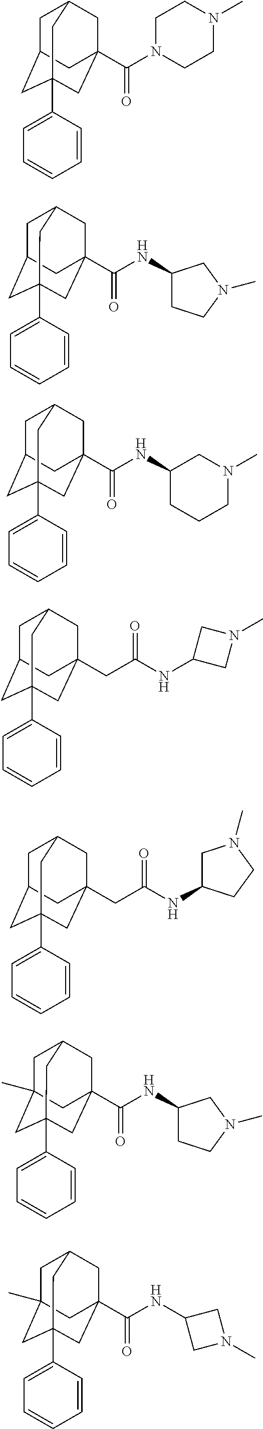

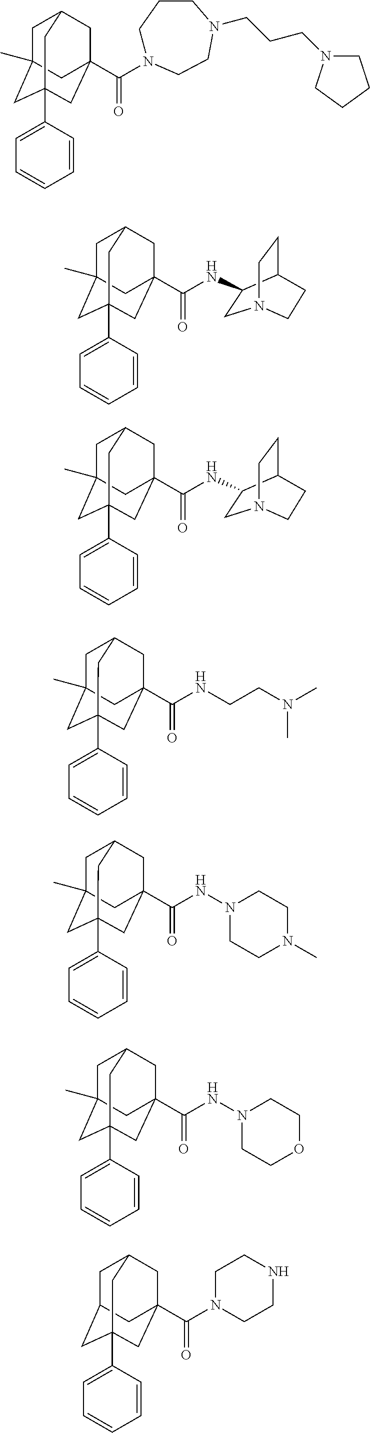

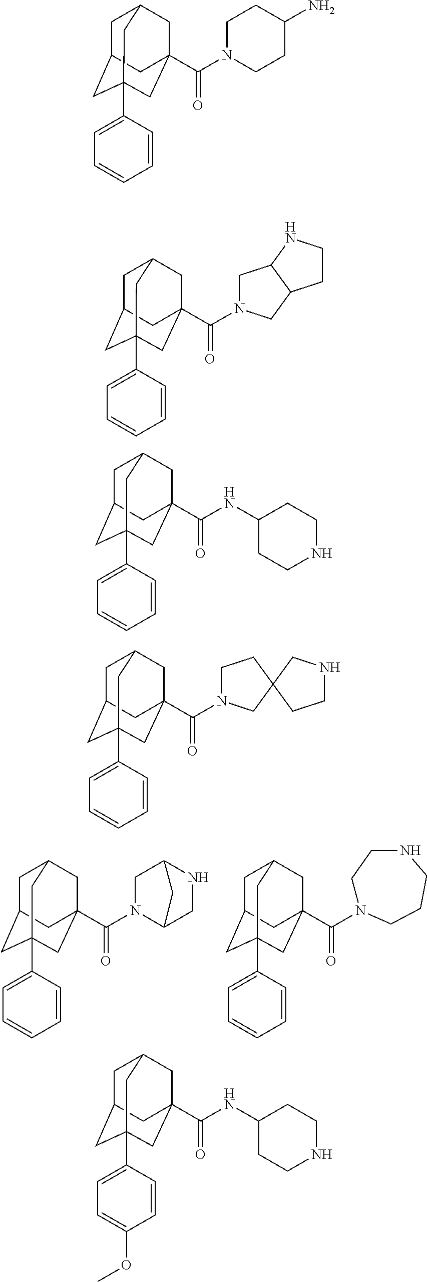

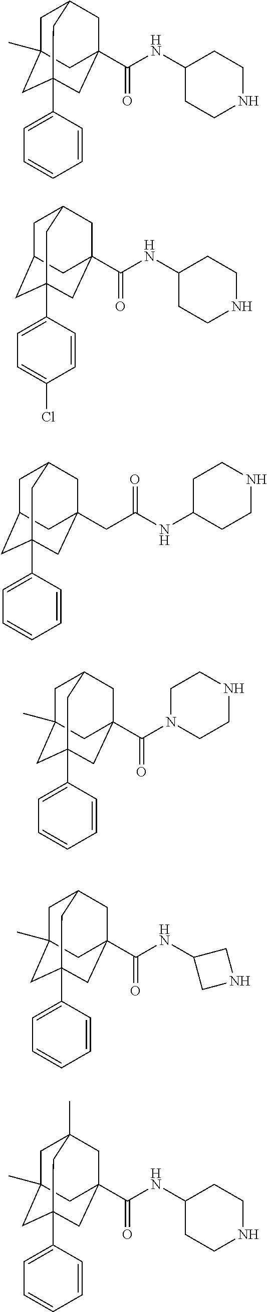

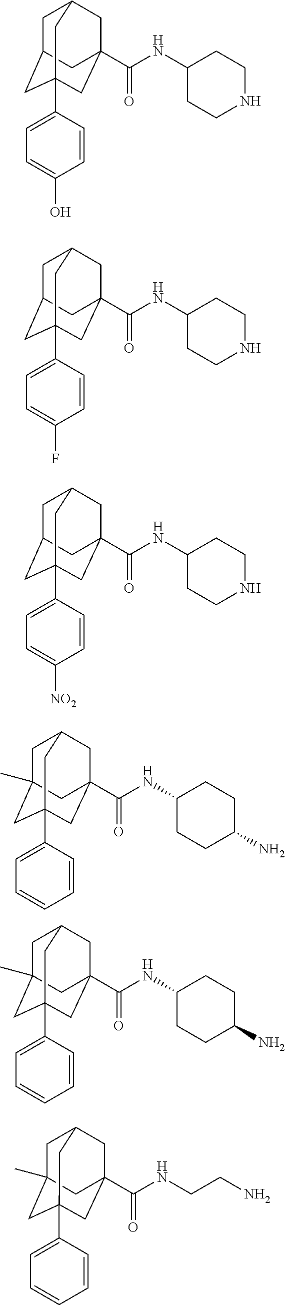

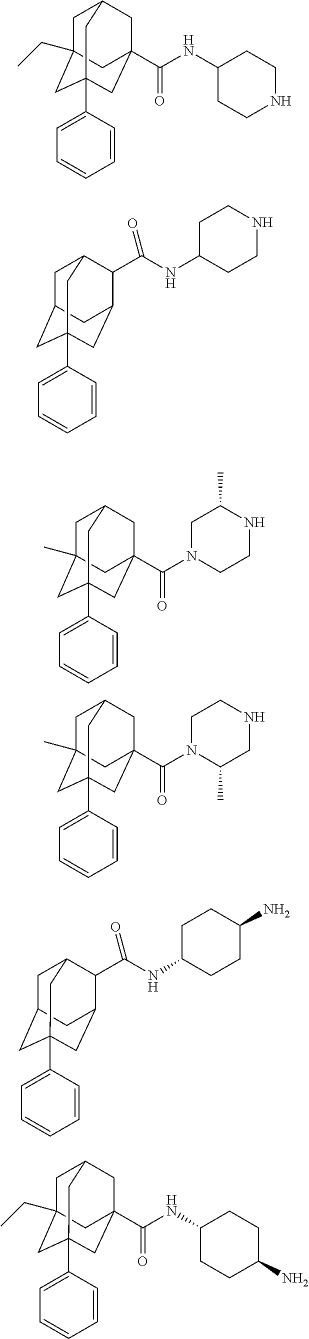

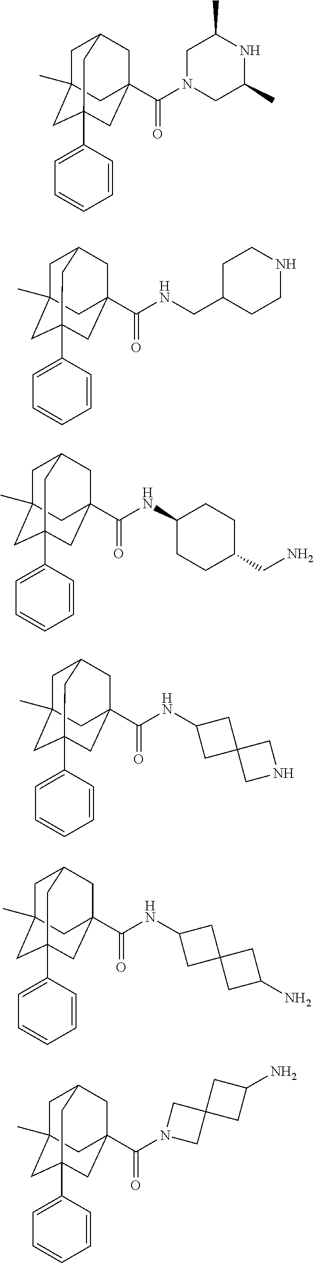

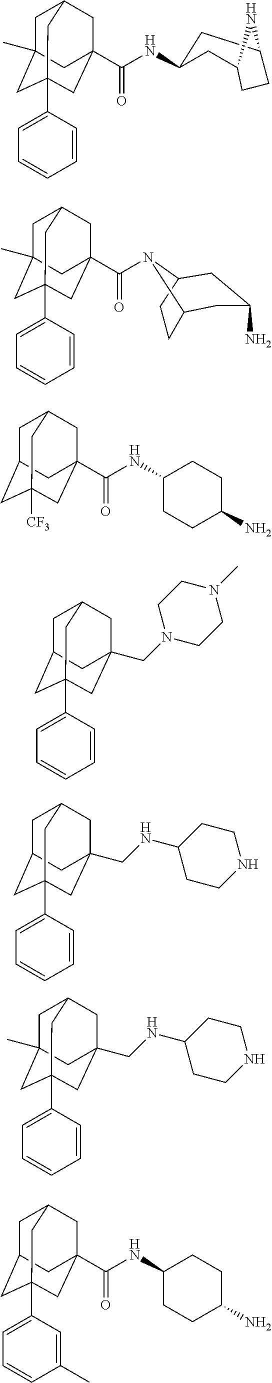

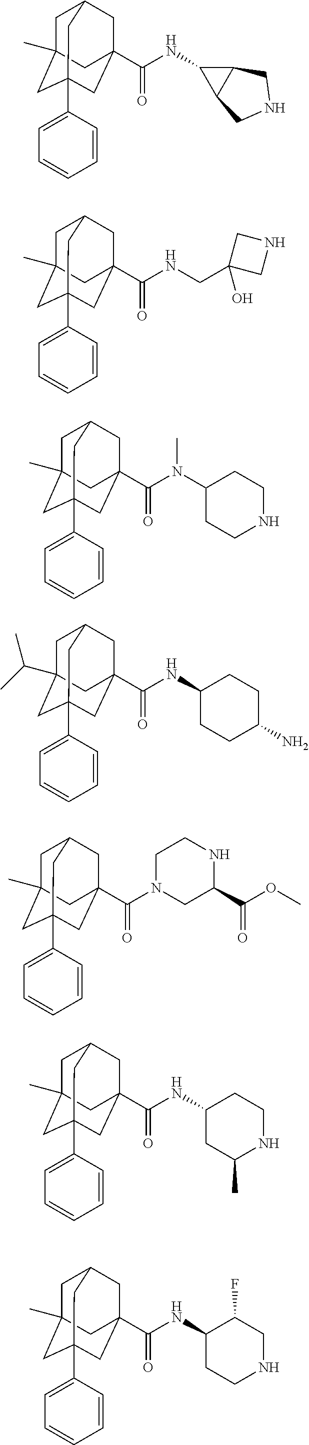

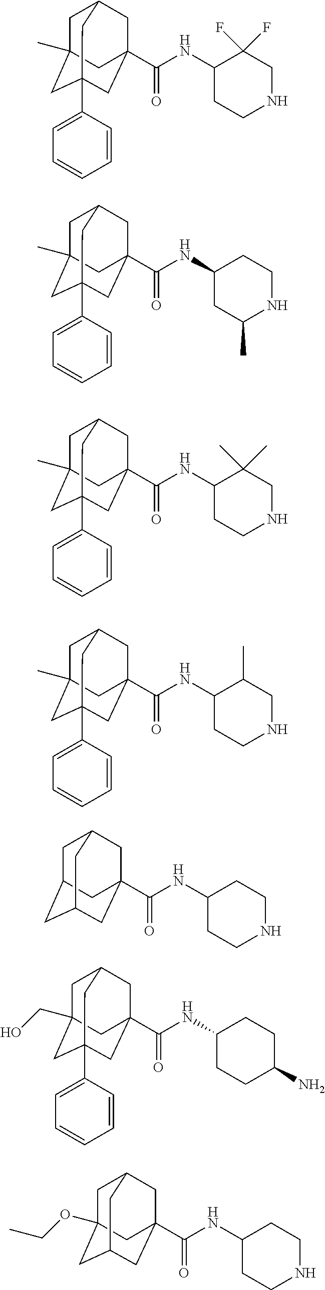

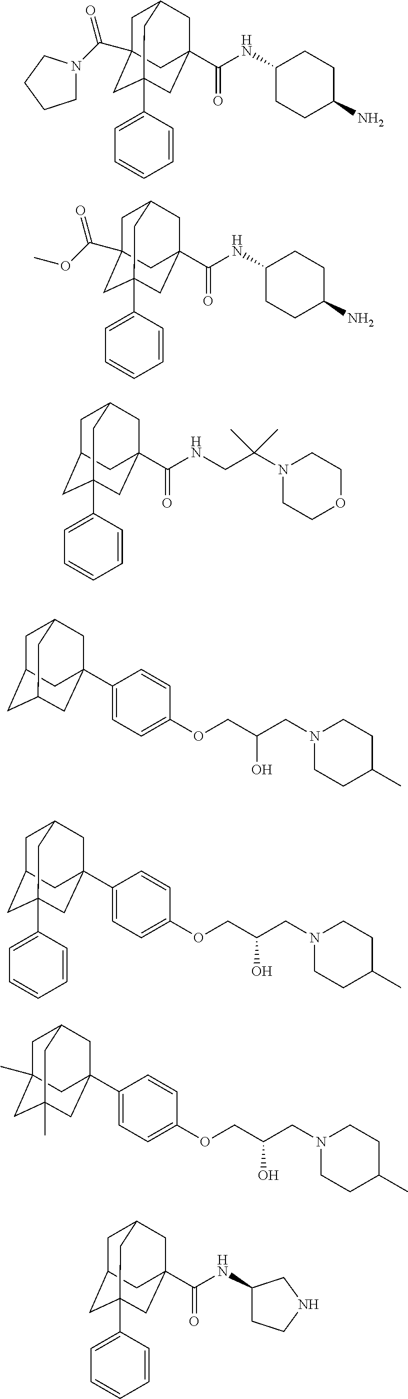

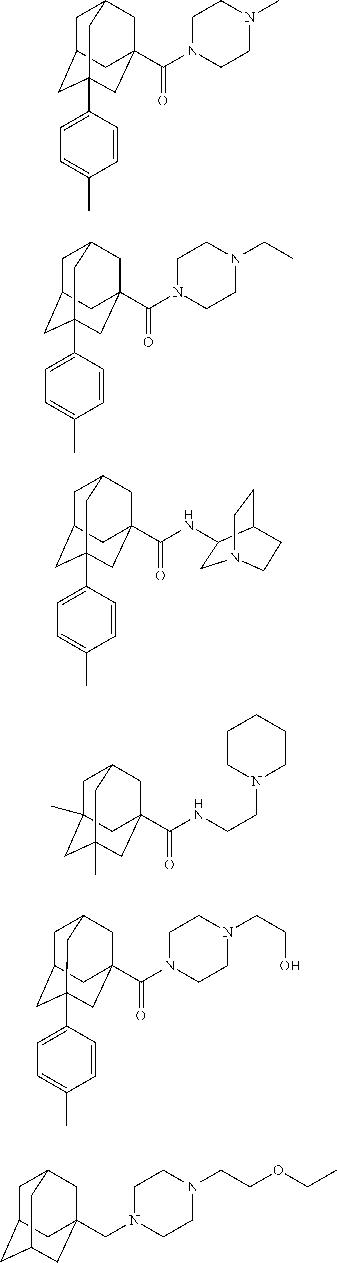

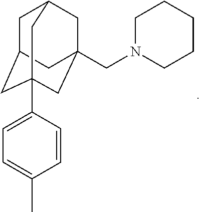 [SMILES: Cc1ccc(C23CC4CC(CC(CN5CCCCC5)(C4)C2)C3)cc1]

24. The compound of claim 23, wherein X is 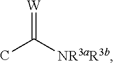
Y is CH₂, W is selected from O and S, and Q is CH₂.

25. The compound of claim 24, wherein:
W is O;
R¹ is phenyl;
NR^{3a}R^{3b} is selected from the group consisting of

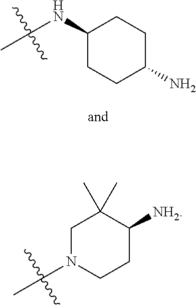

26. The compound of claim 24, wherein:
W is S;
R¹ is phenyl;
R² is selected from the group consisting of halogen, methyl, ethyl, propyl, chloromethyl, chloroethyl, methylthiomethyl, and thiomethyl; and wherein
NR^{3a}R^{3b} is selected from the group consisting of

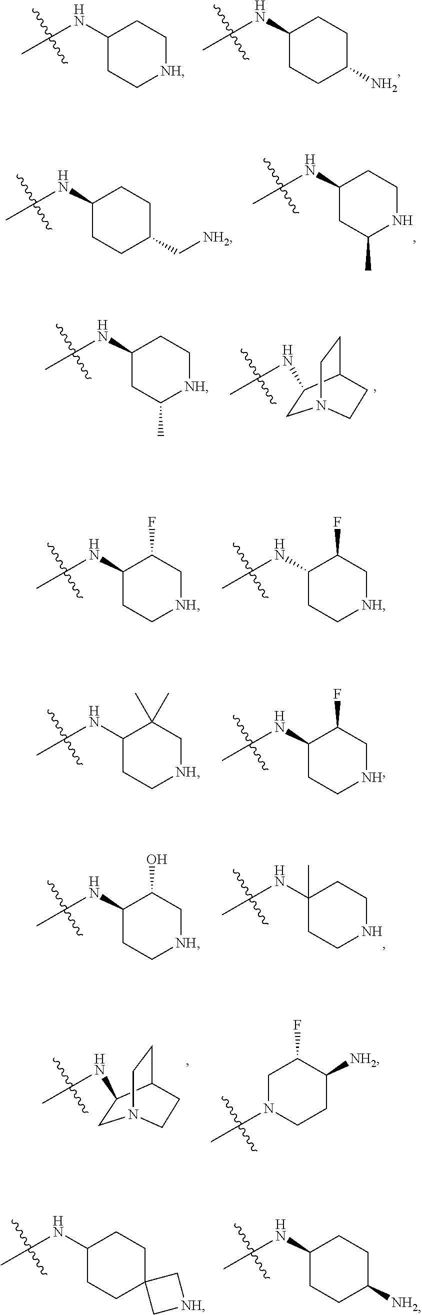

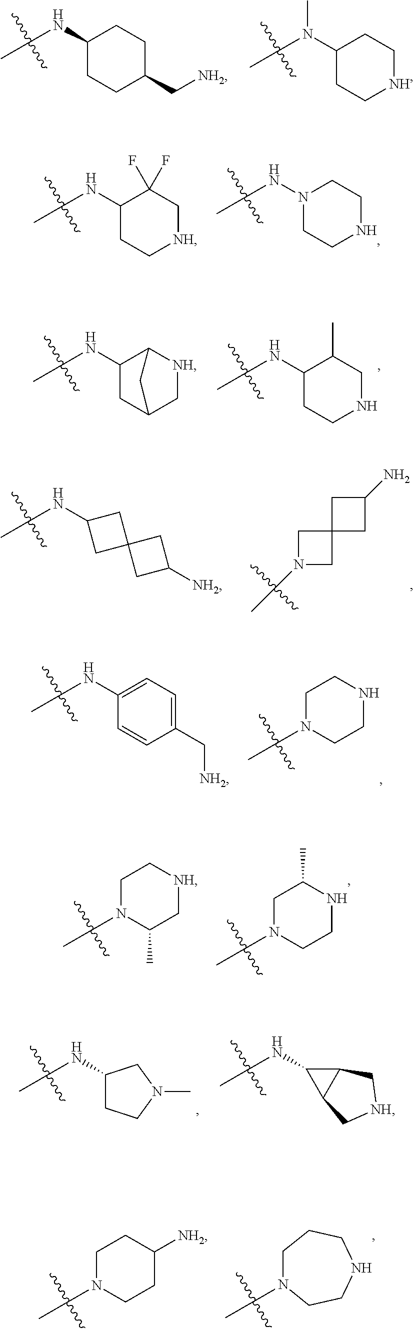

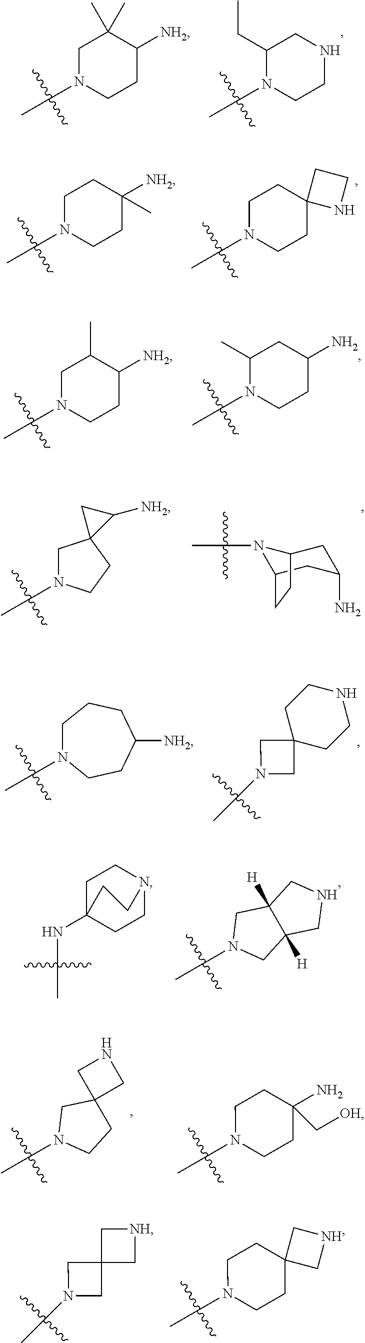

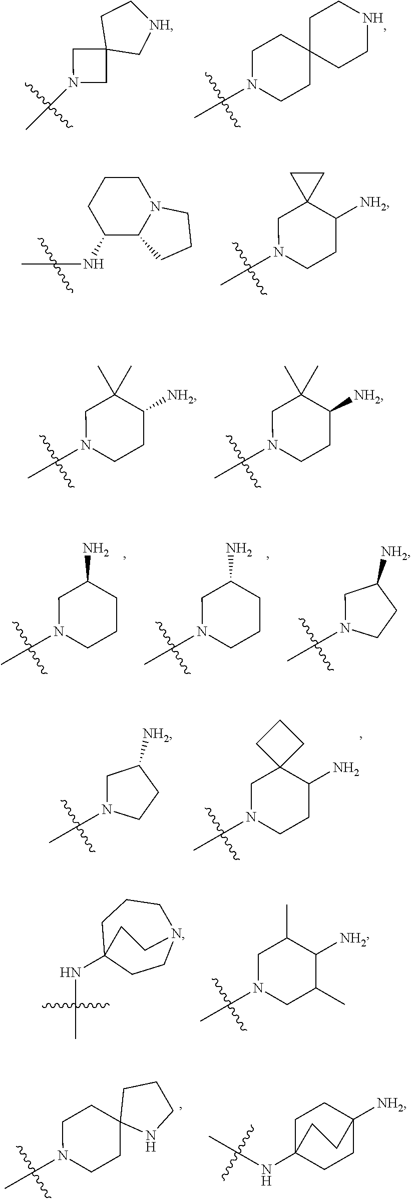

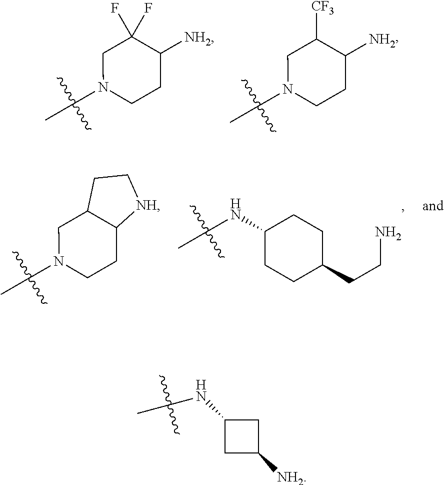 [SMILES: *N1CCC(N)C(F)(F)C1 ; *N1CCC(N)C(C(F)(F)F)C1 ; *N1CCC2NCCC2C1 ; *N[C@H]1CC[C@H](CCN)CC1 ; *N[C@H]1C[C@H](N)C1]

27. The compound of claim 23, wherein X is 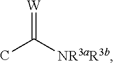
Y is CH₂, W is selected from O and S, and Q is CR²³R²⁴.

28. The compound of claim 27, wherein:
R¹ is phenyl;
NR^{3a}R^{3b} is selected from the group consisting of 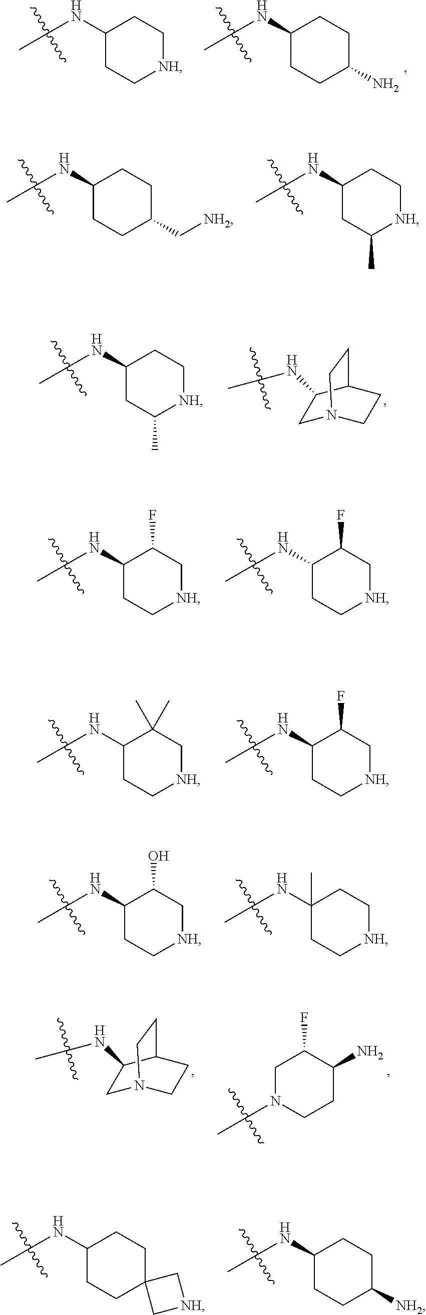 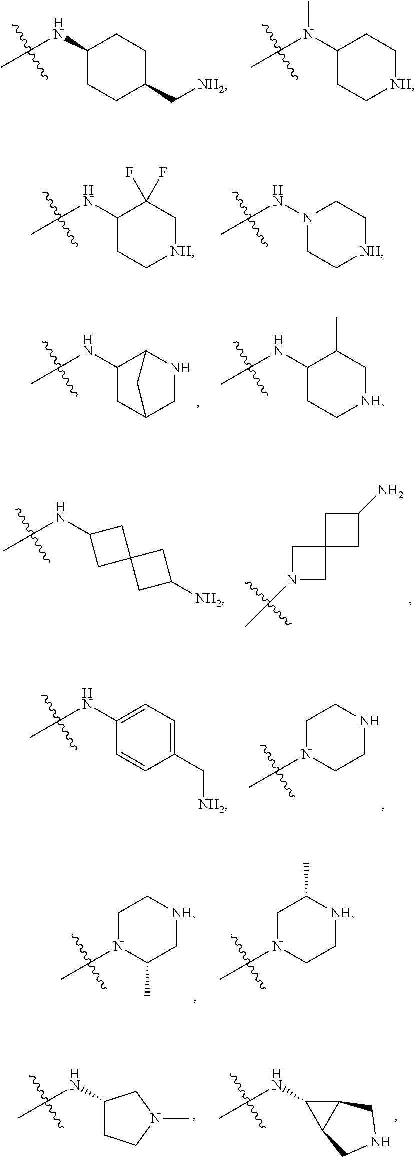 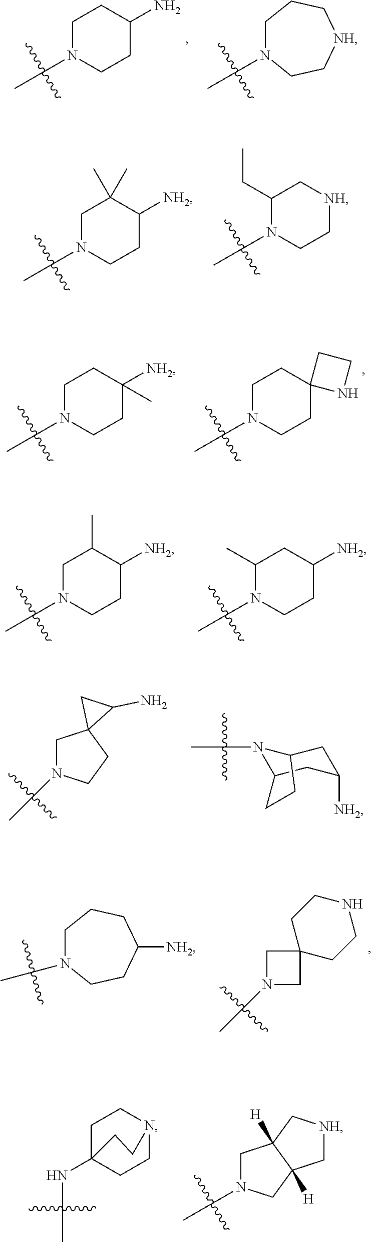 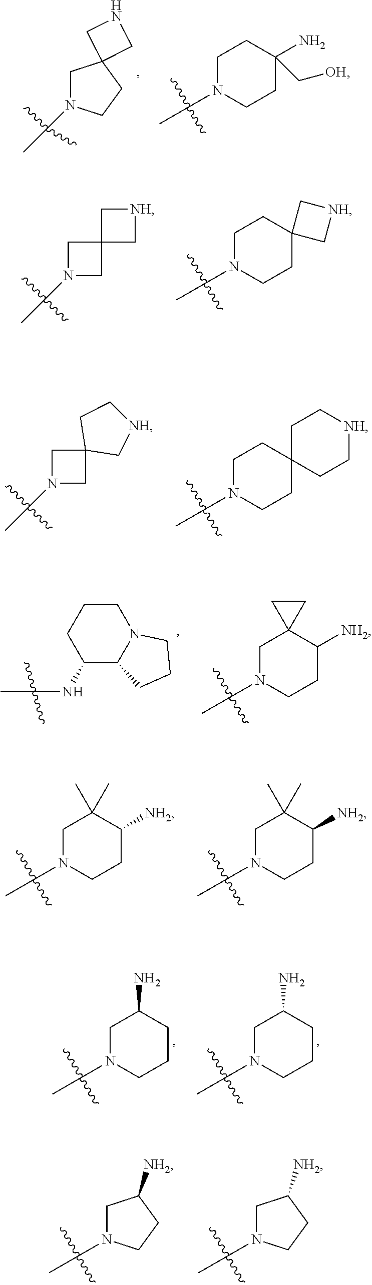 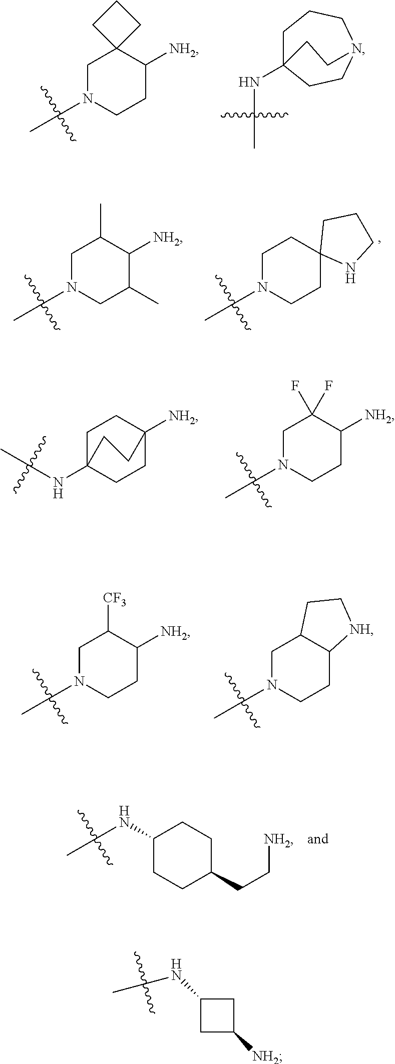
wherein
R²³ is selected from the group consisting of hydrogen, halogen, methyl, ethyl, propyl, butyl, benzyl, phenethyl, methoxy, ethoxy, phenoxy, benzyloxy, chloromethyl, fluoromethyl, difluoromethyl, methylthiomethyl, etylthiomethyl, 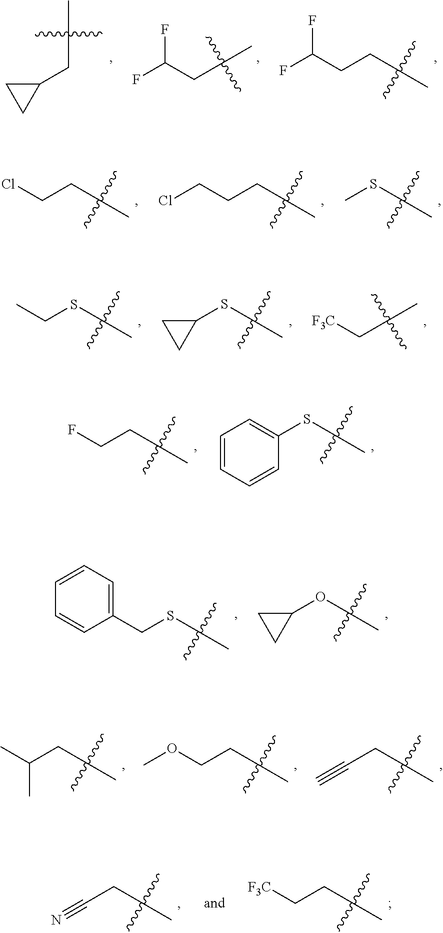
and R²⁴ is selected from the group consisting of halogen, methyl, ethyl, propyl, butyl, benzyl, phenethyl, methoxy, ethoxy, phenoxy, benzyloxy, chloromethyl, fluoromethyl, difluoromethyl, methylthiomethyl, etylthiomethyl, 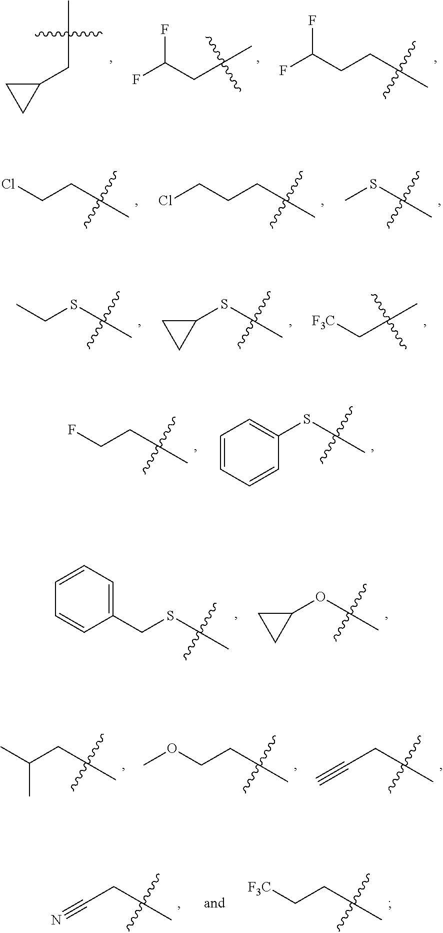
or R²³ and R²⁴ may be taken together with the carbon atom to which they are attached to form a cyclopropyl, cyclobutyl, or cyclopentyl;
or R²³ and R²⁴ may be taken together with the carbon atom to which they are attached to form a carbonyl,

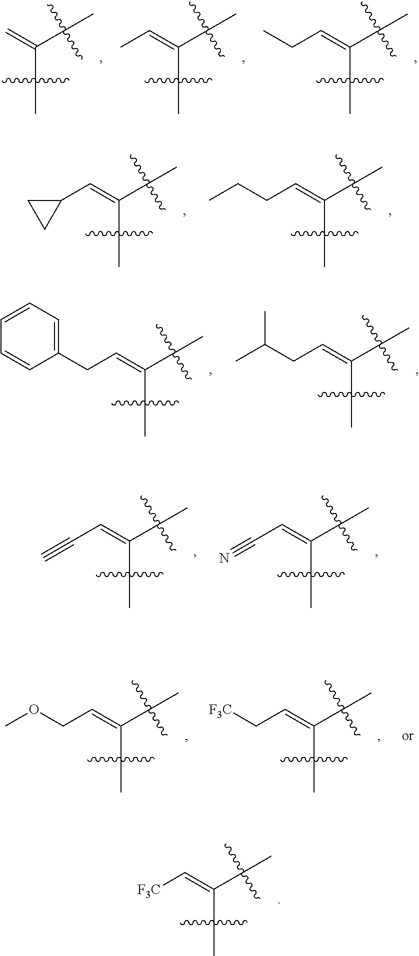

29. The compound of claim 23, wherein X is CH, Y is 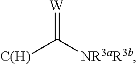
W is selected from O and S, and Q is CH₂.

30. The compound of claim 29, wherein:
W is S or O;
R¹ is phenyl;
R² is selected from the group consisting of halogen, methyl, ethyl, propyl, chloromethyl, chloroethyl, methylthiomethyl, and thiomethyl; and wherein
NR^{3a}R^{3b} is selected from the group consisting of

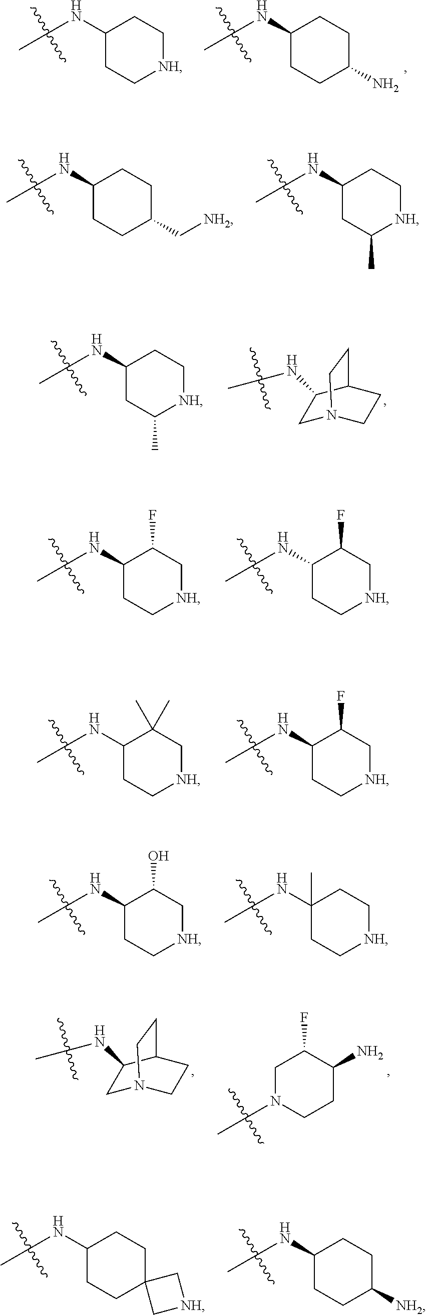

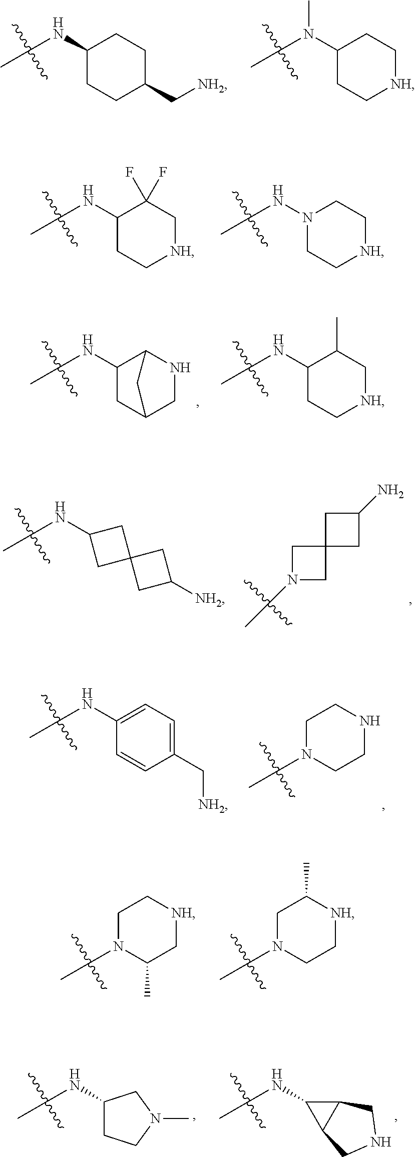

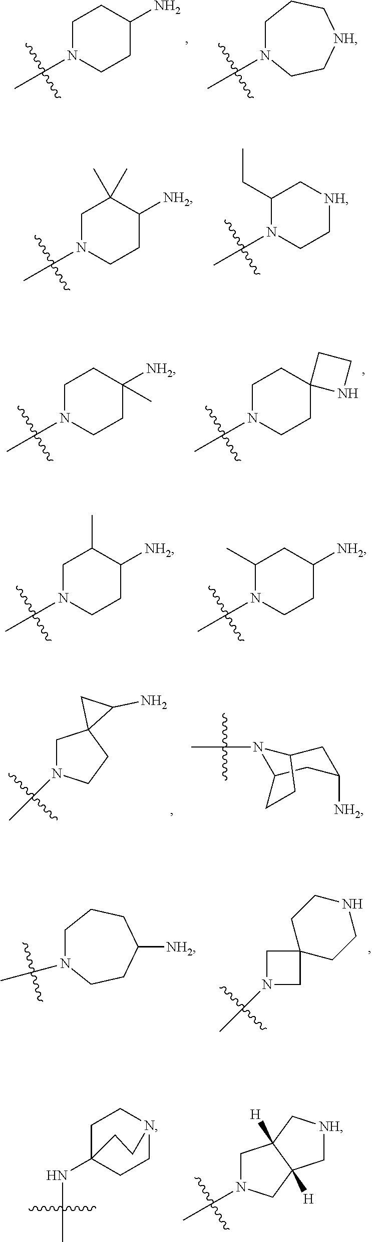

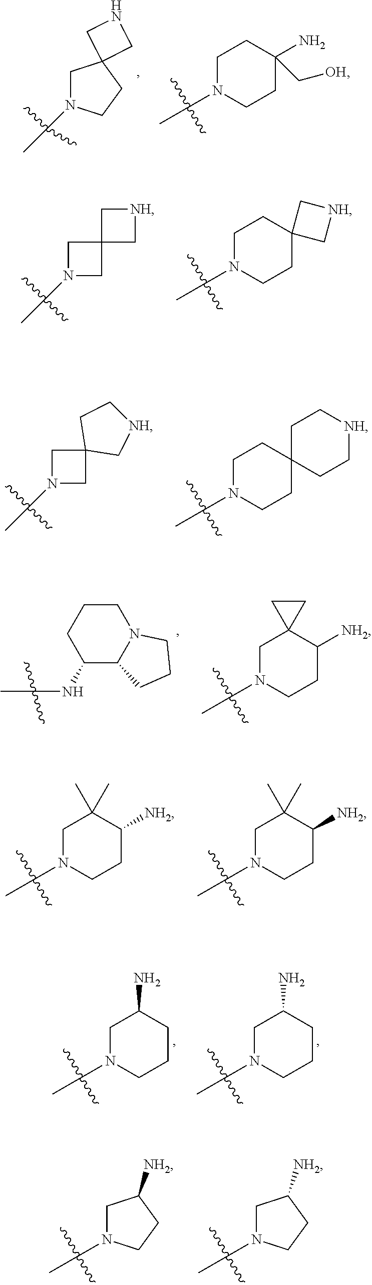

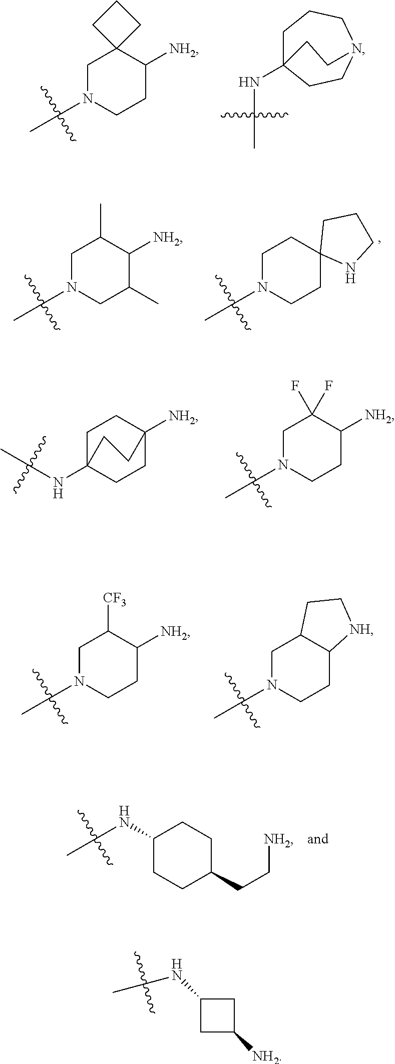

31. The compound of claim 23, including administering a therapeutic amount of a therapeutic agent selected from the group consisting of Ribavirin, viral RNA-dependent-RNA polymerase inhibitors including favipiravir, Triazavirin, Remdesivir (GS-5734), monoclonal antibody therapies including, ZMapp, REGN3470-3471-3479, mAb 114, vaccines including, cAd3-EBOZ, rVSV-ZEBOV, small interfering RNAs and microRNAs and immunomodulators.

32. The compound of claim 23, wherein Structural Formula I is represented by the following enantiomerically pure compounds of Structural Formulae II and III 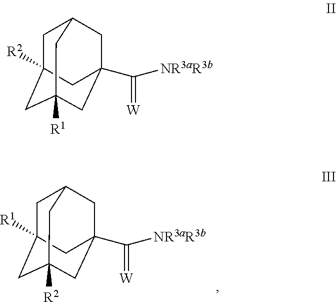
or a pharmaceutically acceptable salt, and a pharmaceutically acceptable carrier, diluent, or vehicle thereof.

33. The compound of claim 32, wherein:
W is O;
R¹ is phenyl;
NR^{3a}R^{3b} is selected from the group consisting of

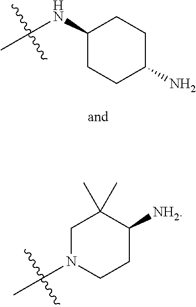

34. The compound of claim 32, wherein:
W is S;
R¹ is phenyl;
R² is selected from the group consisting of halogen, methyl, ethyl, propyl, chloromethyl, chloroethyl, methylthiomethyl, and thiomethyl; and wherein
NR^{3a}R^{3b} is selected from the group consisting of

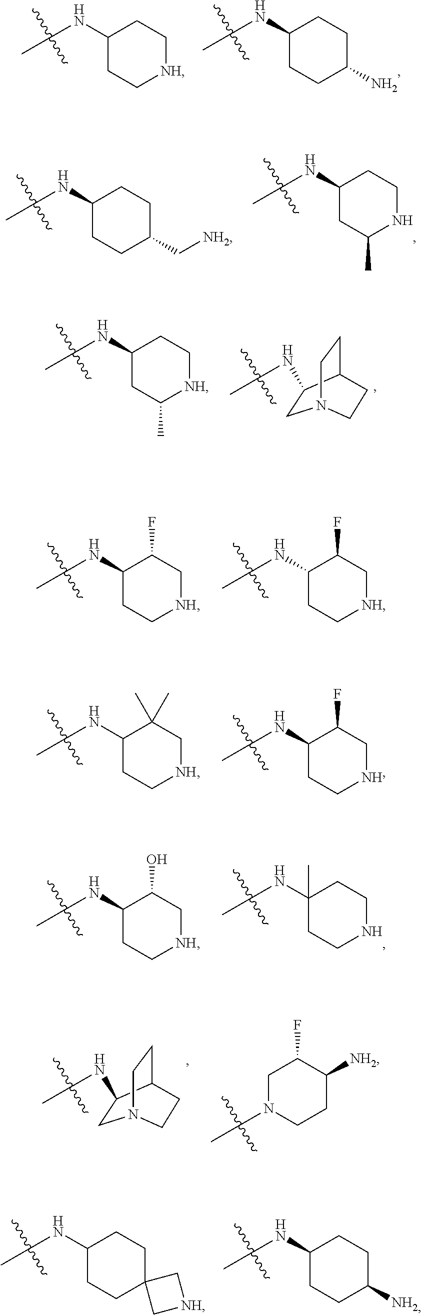

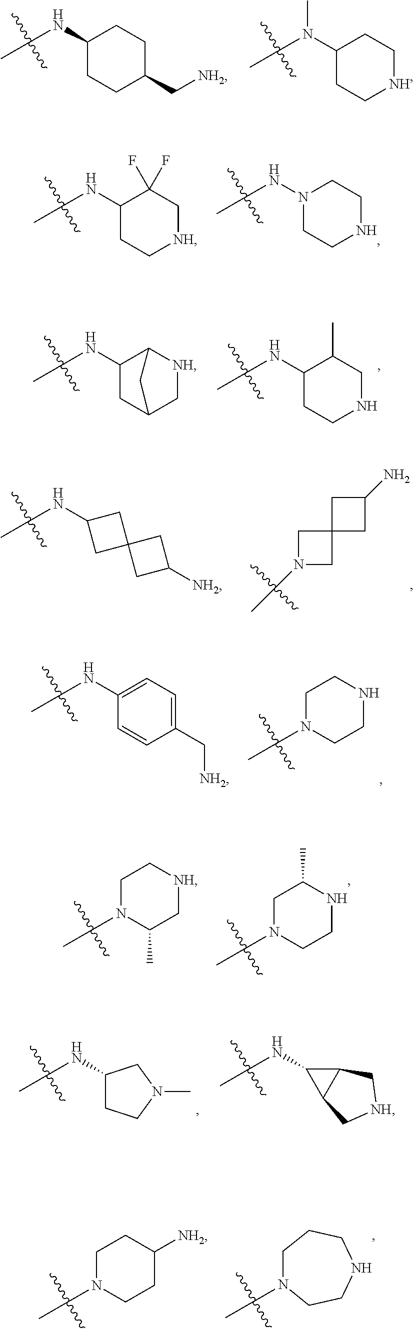

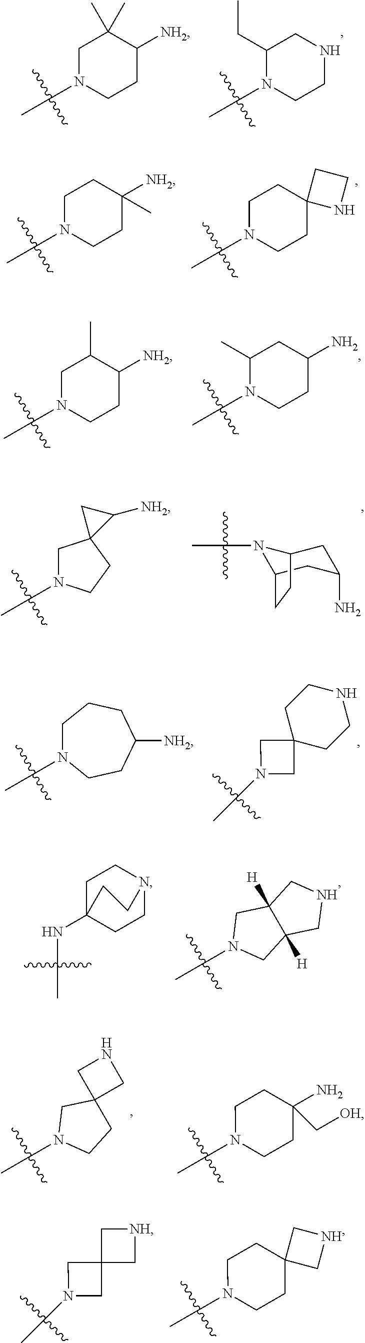

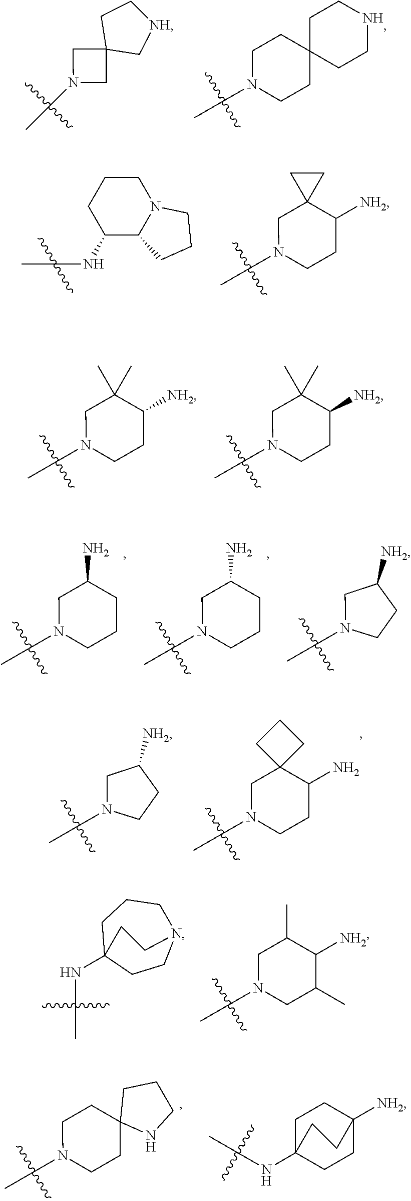

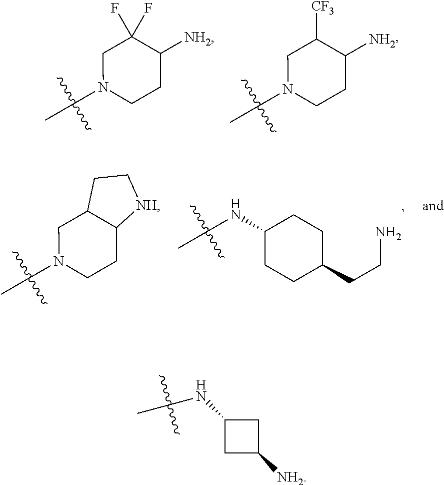 [SMILES: *N1CCC(N)C(F)(F)C1 ; *N1CCC(N)C(C(F)(F)F)C1 ; *N1CCC2NCCC2C1 ; *N[C@H]1CC[C@H](CCN)CC1 ; *N[C@H]1C[C@H](N)C1]

35. The compound of claim 32, including administering a therapeutic amount of a therapeutic agent selected from the group consisting of Ribavirin, viral RNA-dependent-RNA polymerase inhibitors including favipiravir, Triazavirin, Remdesivir (GS-5734), monoclonal antibody therapies including, ZMapp, REGN3470-3471-3479, mAb 114, vaccines including, cAd3-EBOZ, rVSV-ZEBOV, small interfering RNAs and microRNAs and immunomodulators.

36. The compound of claim 23, wherein Structural Formula I is represented by the following compounds of Structural Formulae IV, V, and VI 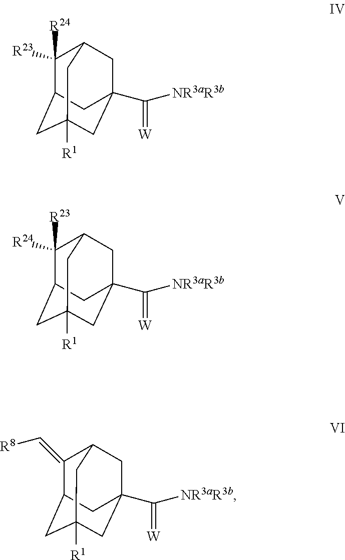
or a pharmaceutically acceptable salt, and a pharmaceutically acceptable carrier, diluent, or vehicle thereof.

37. The compound of claim 36, wherein:
W is O or S;
R¹ is phenyl;
NR^{3a}R^{3b} is selected from the group consisting of 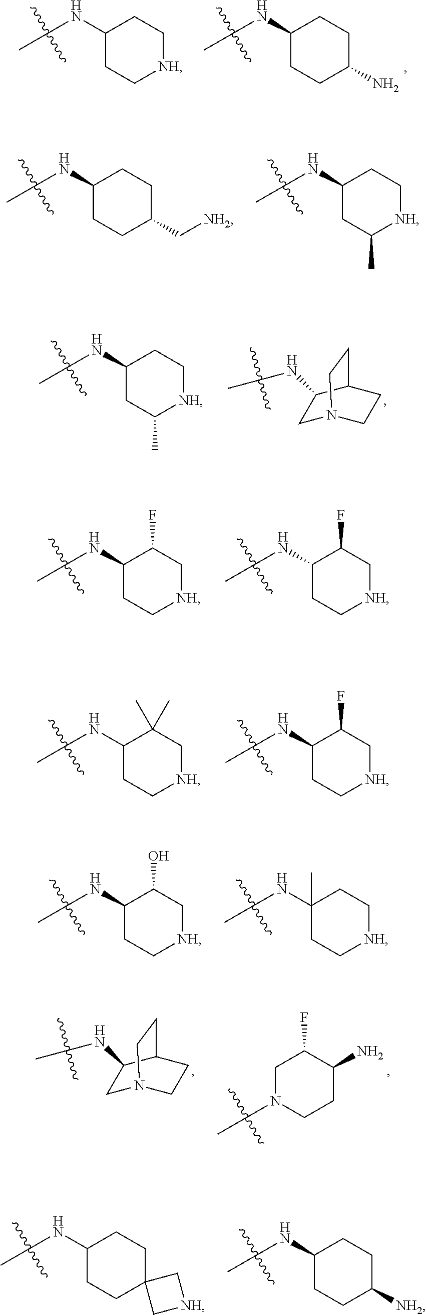 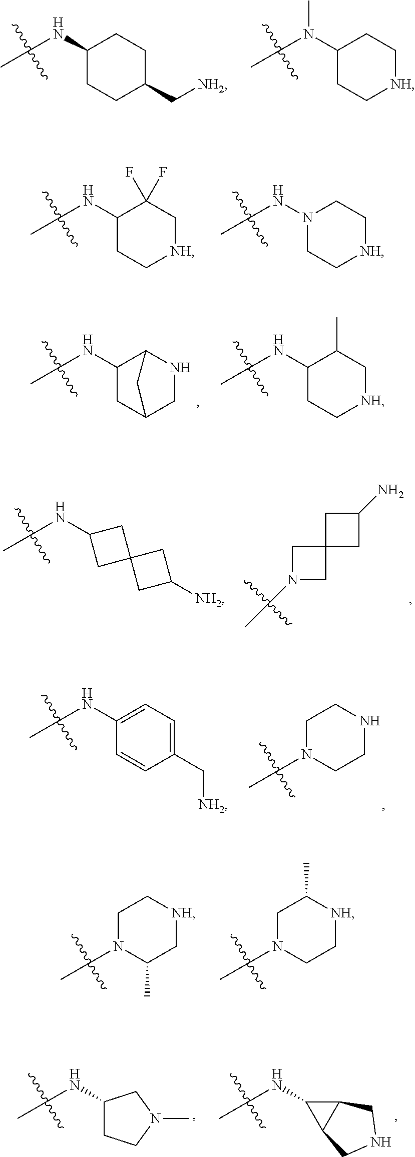 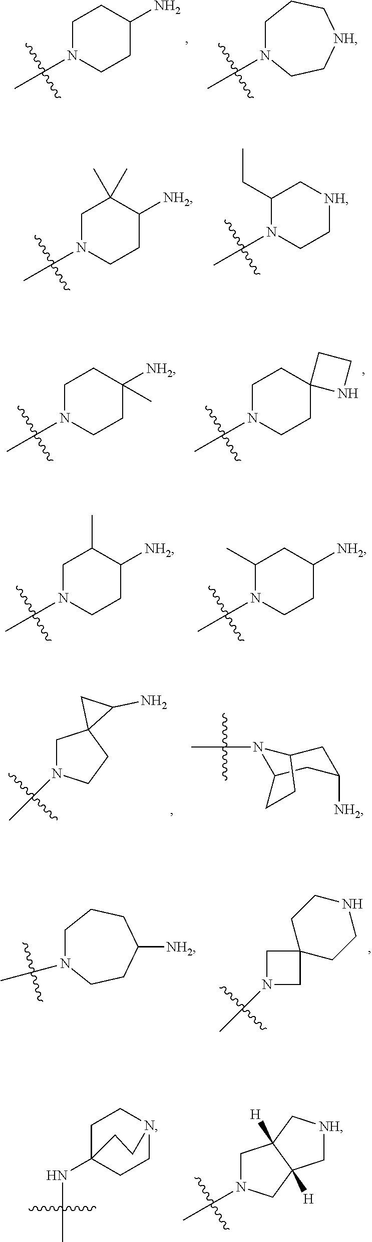 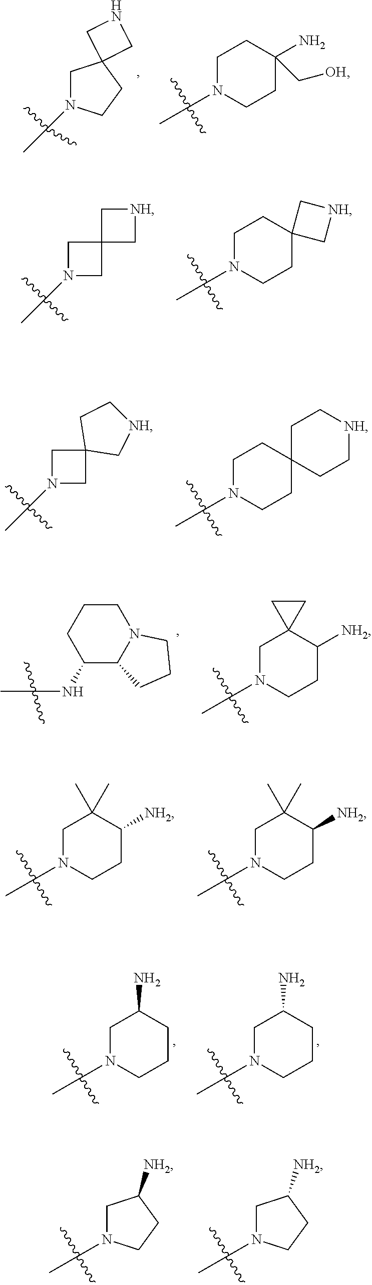 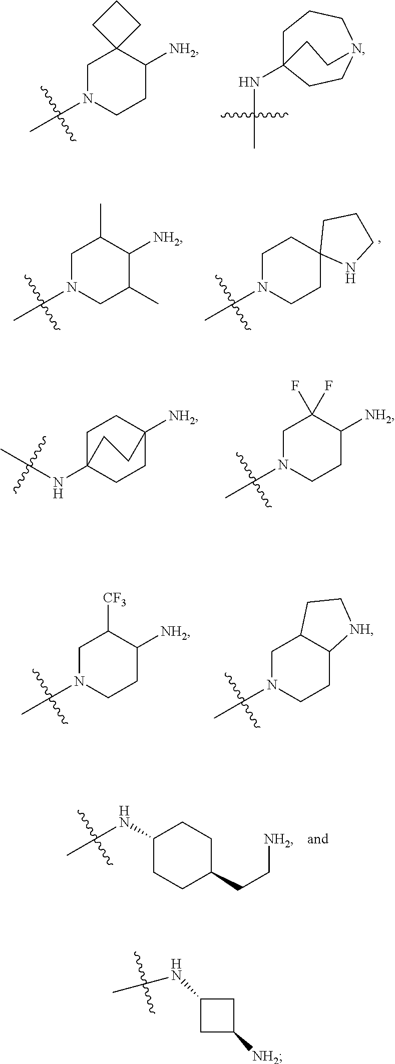
and
wherein
R²³ is selected from the group consisting of hydrogen, halogen, methyl, ethyl, propyl, butyl, benzyl, phenethyl, methoxy, ethoxy, phenoxy, benzyloxy, chloromethyl, fluoromethyl, difluoromethyl, methylthiomethyl, etylthiomethyl, 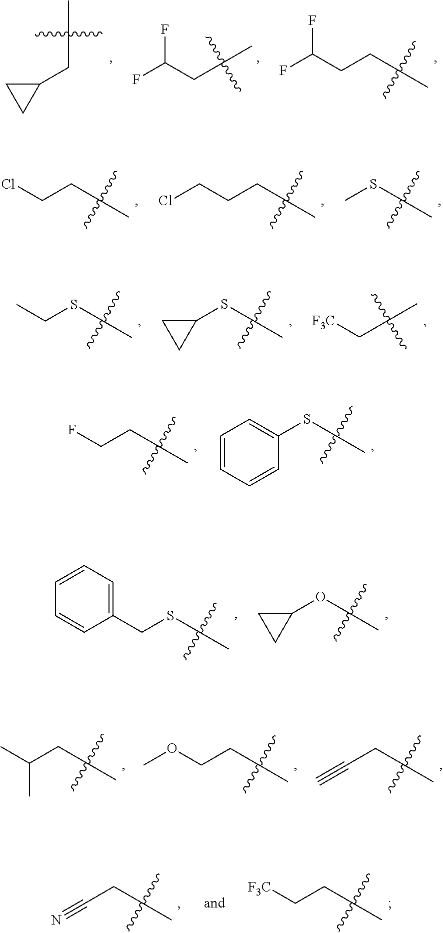
and R²⁴ is selected from the group consisting of halogen, methyl, ethyl, propyl, butyl, benzyl, phenethyl, methoxy, ethoxy, phenoxy, benzyloxy, chloromethyl, fluoromethyl, difluoromethyl, methylthiomethyl, etylthiomethyl, 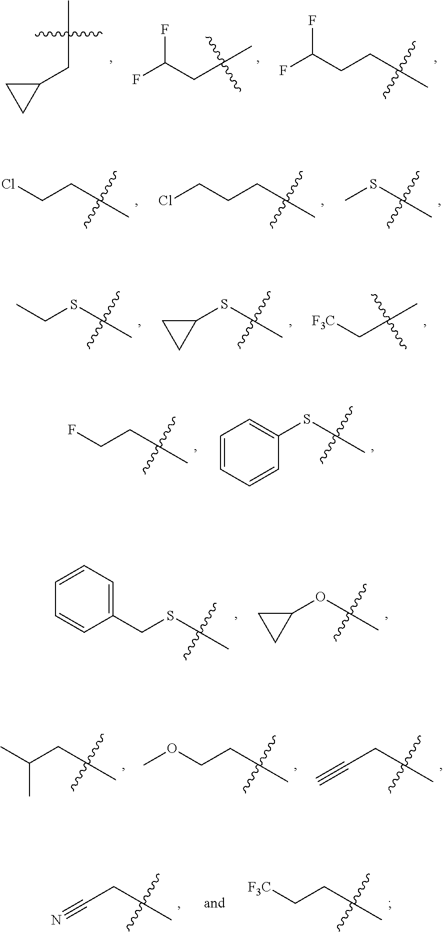
or R²³ and R²⁴ may be taken together with the carbon atom to which they are attached to form a cyclopropyl, cyclobutyl, or cyclopentyl;
and wherein R⁸ is selected from the group consisting of hydrogen, methyl, ethyl, propyl, cyclopropyl, benzyl, cyano, ethynyl, methoxymethyl, trifluoromethyl, trifluoroethyl, and

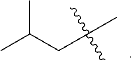 [SMILES: *CC(C)C]

38. The compound of claim 36, wherein Structural Formula VI is represented by the following enantiomerically pure compounds of Structural Formulae Via and VIb 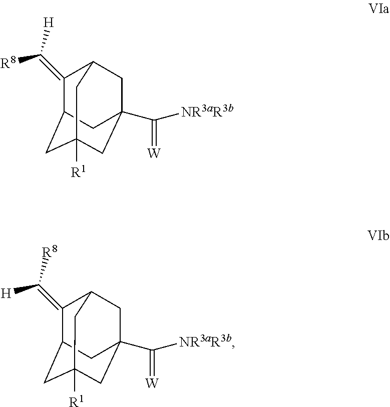
or a pharmaceutically acceptable salt, and a pharmaceutically acceptable carrier, diluent, or vehicle thereof, wherein:
R⁸ is selected from the group consisting of methyl, ethyl, propyl, cyclopropyl, benzyl, cyano, ethynyl, methoxymethyl, trifluoromethyl, trifluoroethyl, and

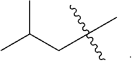 [SMILES: *CC(C)C]

39. The compound of claim 36, including administering a therapeutic amount of a therapeutic agent selected from the group consisting of Ribavirin, viral RNA-dependent-RNA polymerase inhibitors including favipiravir, Triazavirin, Remdesivir (GS-5734), monoclonal antibody therapies including, ZMapp, REGN3470-3471-3479, mAb 114, vaccines including, cAd3-EBOZ, rVSV-ZEBOV, small interfering RNAs and microRNAs and immunomodulators.

40. The compound of claim 23, wherein Structural Formula I is represented by the following compounds of Structural Formulae VII and VIII 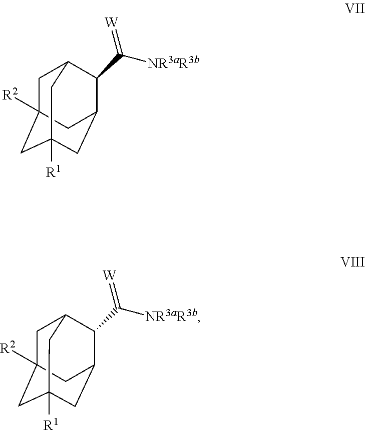
or a pharmaceutically acceptable salt, and a pharmaceutically acceptable carrier, diluent, or vehicle thereof.

41. The compound of claim 40, wherein:
W is O or S;
R¹ is phenyl; and wherein
NR^{3a}R^{3b} is selected from the group consisting of

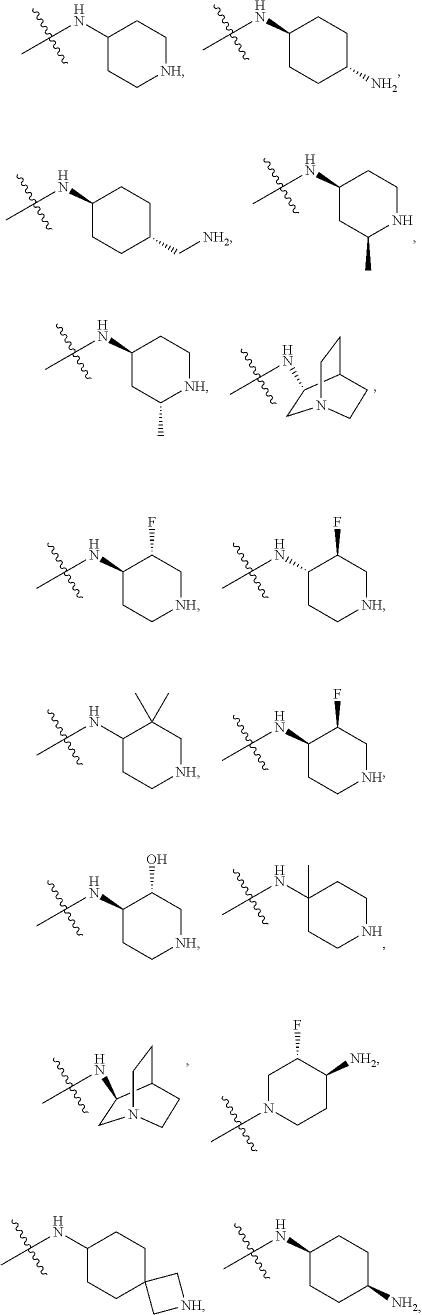

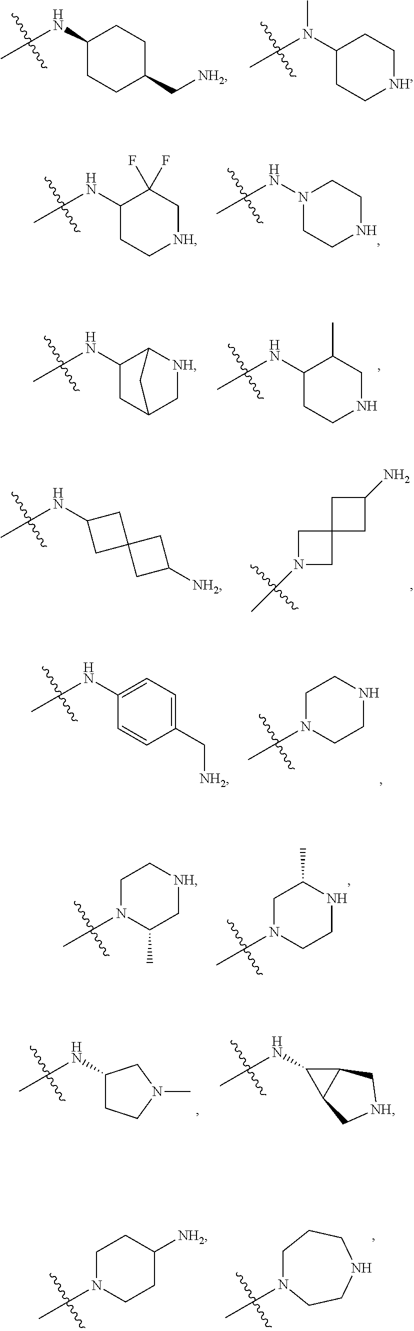

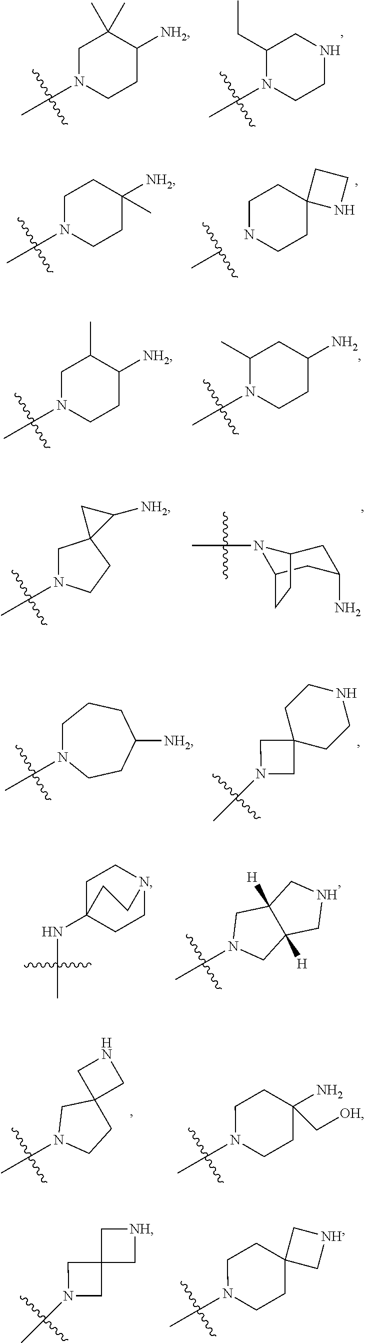

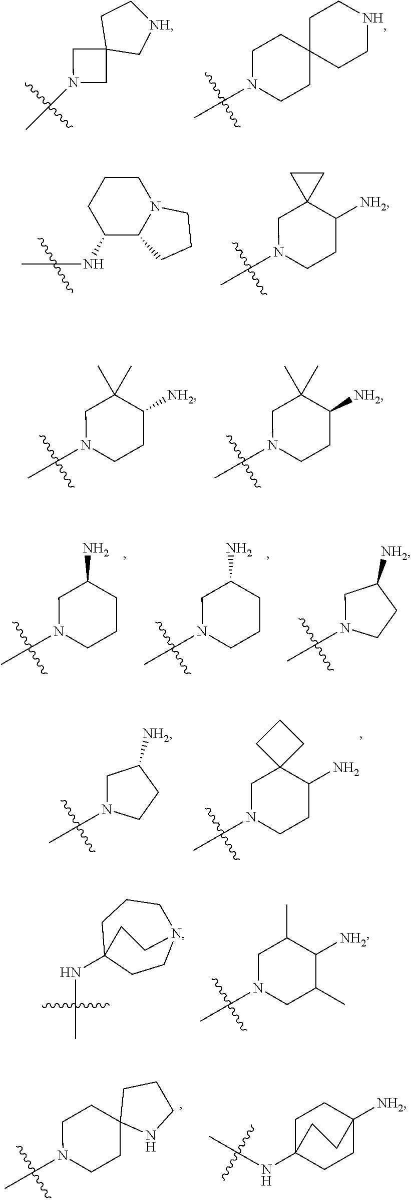

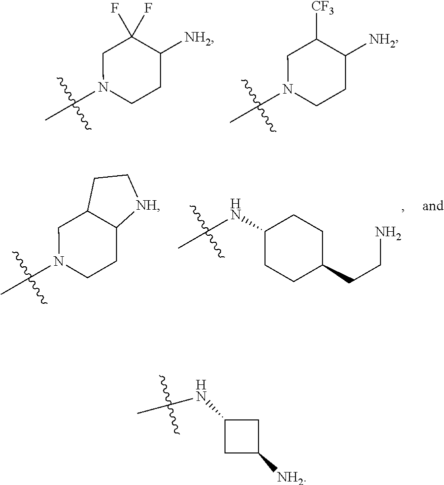 [SMILES: *N1CCC(N)C(F)(F)C1 ; *N1CCC(N)C(C(F)(F)F)C1 ; *N1CCC2NCCC2C1 ; *N[C@H]1CC[C@H](CCN)CC1 ; *N[C@H]1C[C@H](N)C1]

42. A compound selected from the group consisting of:

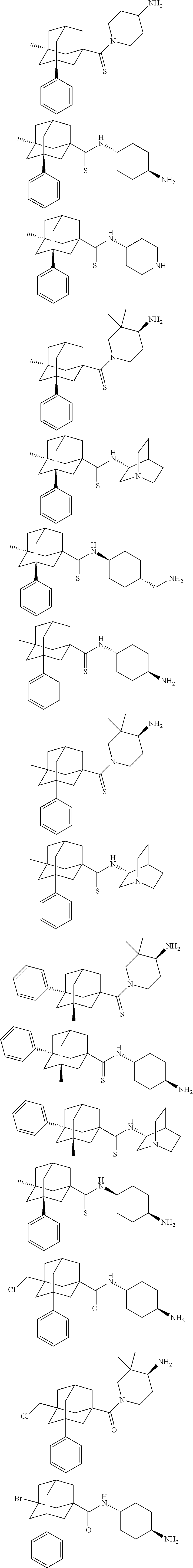

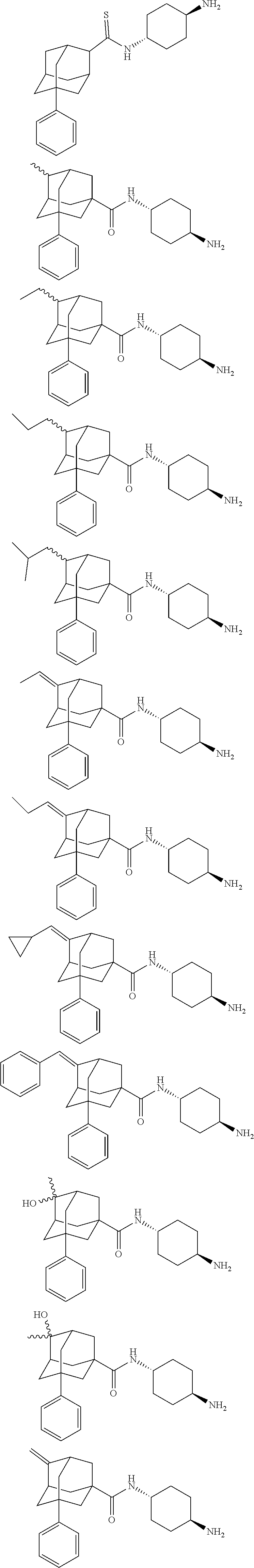

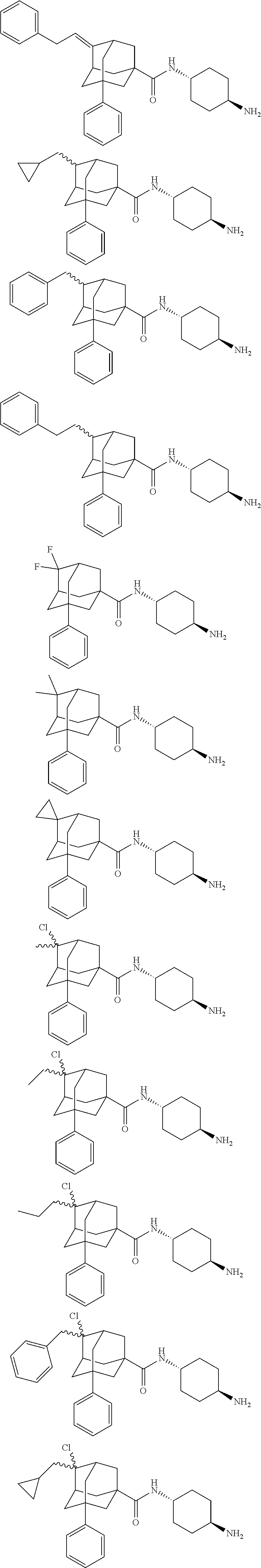

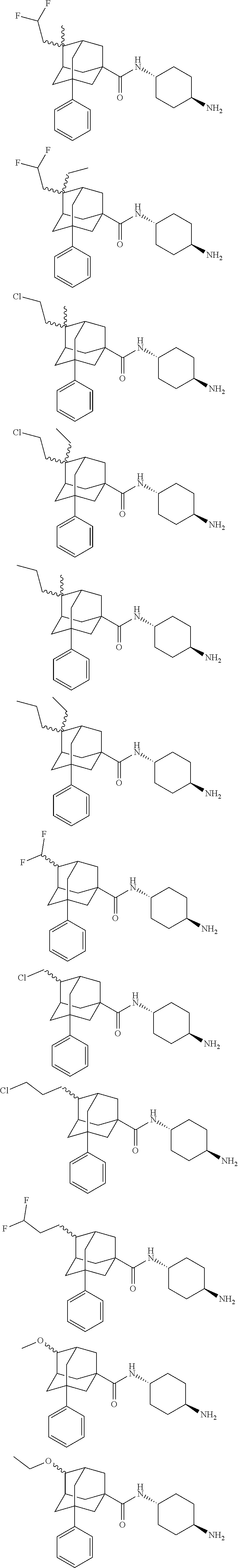

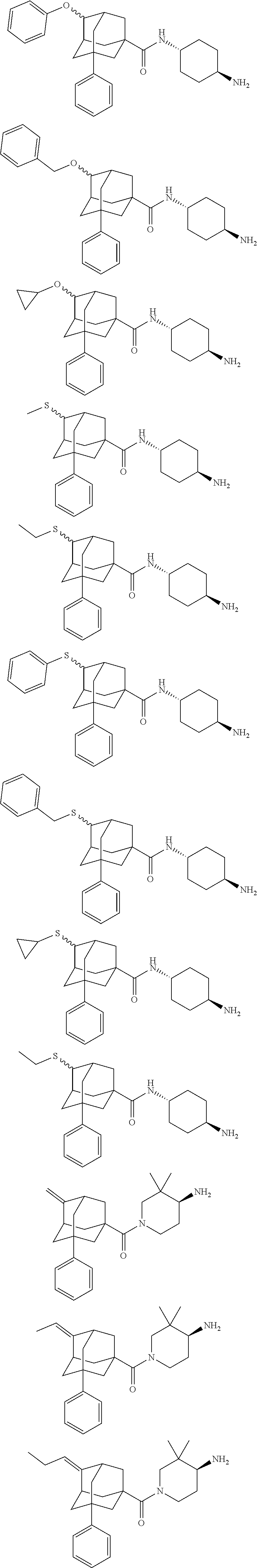

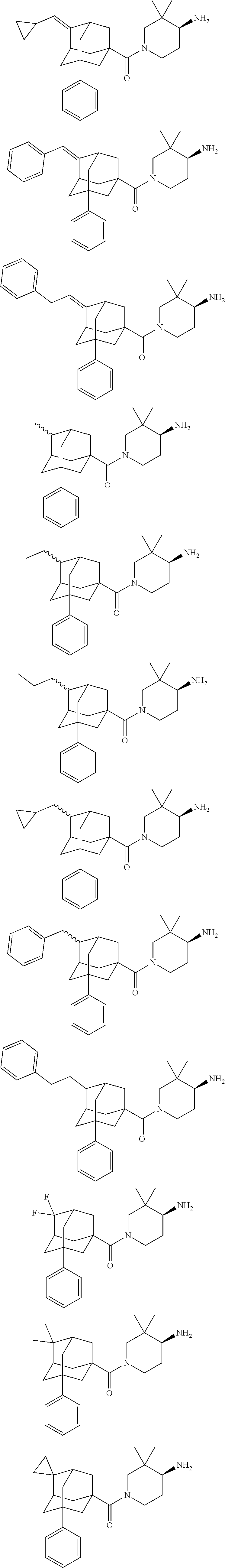

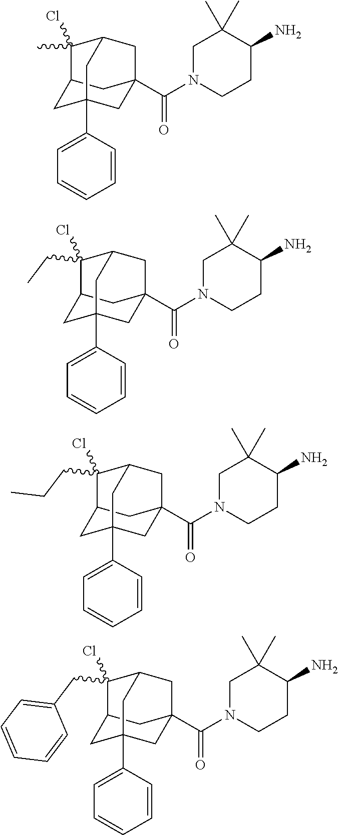

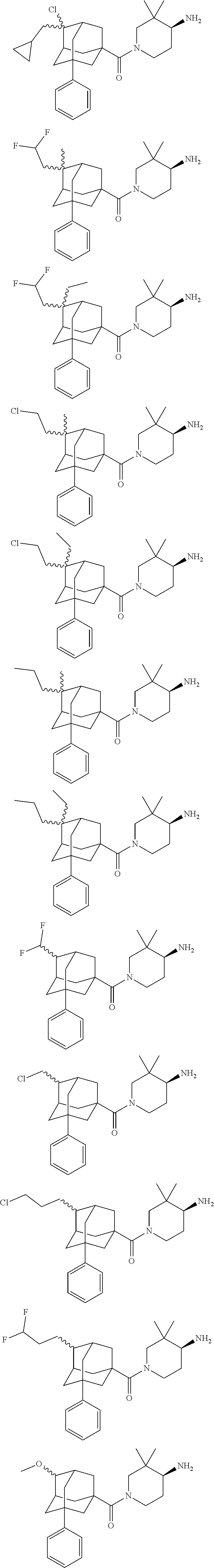

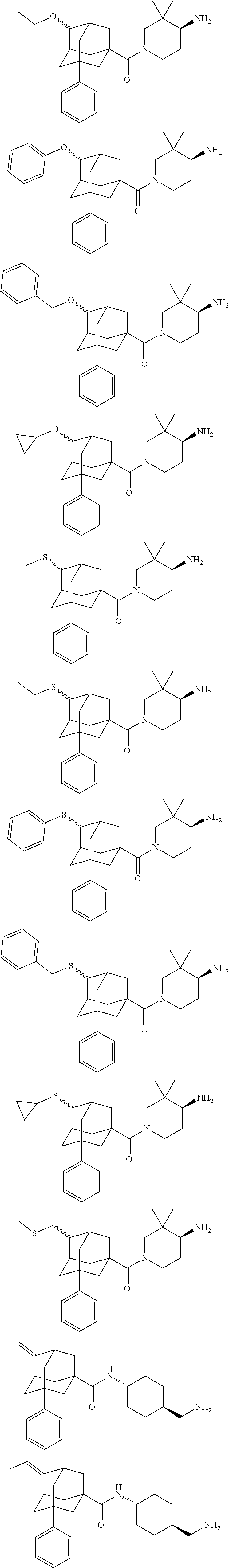

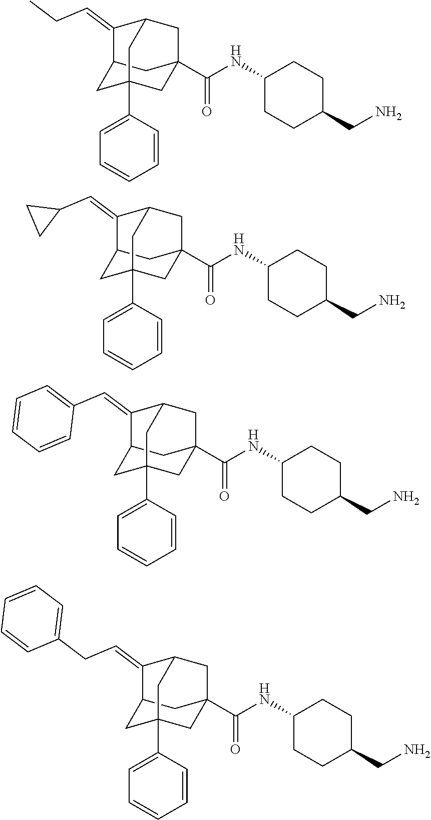

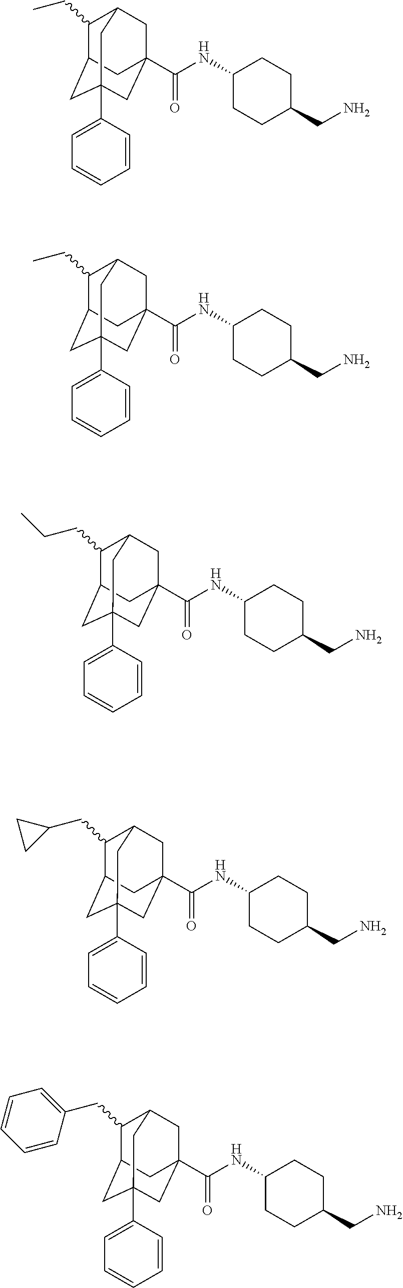 [SMILES: CCC1C2CC3(C(=O)N[C@H]4CC[C@H](CN)CC4)CC1CC(c1ccccc1)(C2)C3 ; CCC1C2CC3(C(=O)N[C@H]4CC[C@H](CN)CC4)CC1CC(c1ccccc1)(C2)C3 ; CCCC1C2CC3(C(=O)N[C@H]4CC[C@H](CN)CC4)CC1CC(c1ccccc1)(C2)C3 ; NC[C@H]1CC[C@H](NC(=O)C23CC4CC(c5ccccc5)(CC(C2)C4CC2CC2)C3)CC1 ; NC[C@H]1CC[C@H](NC(=O)C23CC4CC(c5ccccc5)(CC(C2)C4Cc2ccccc2)C3)CC1]

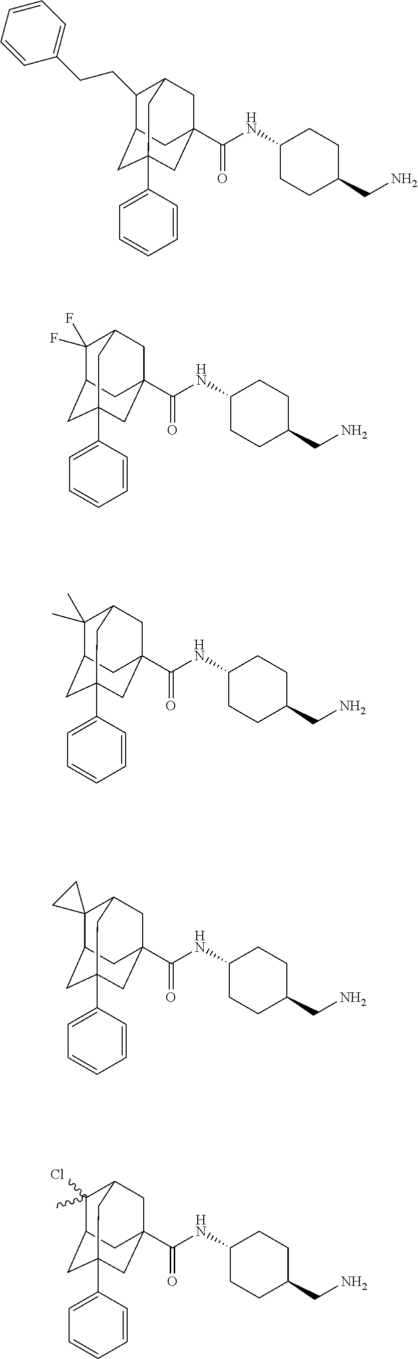

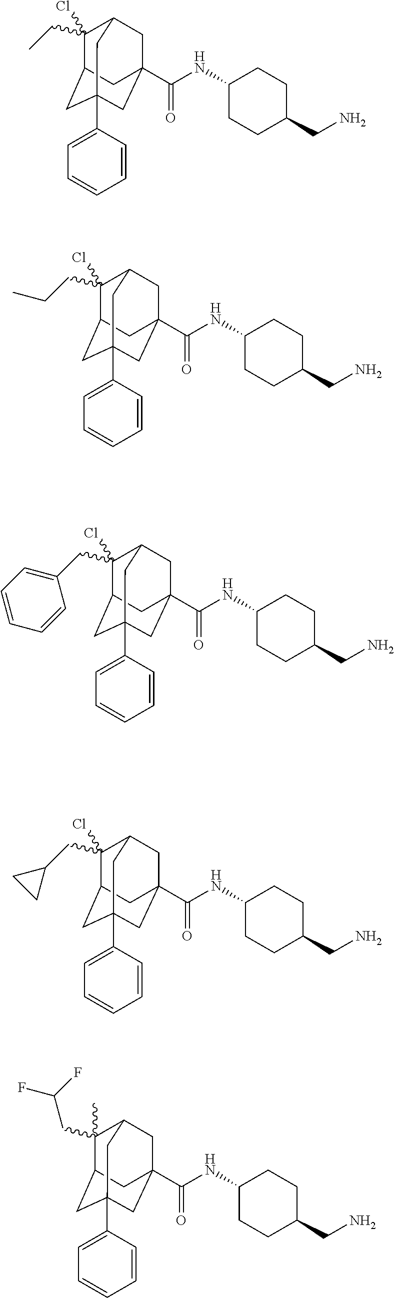

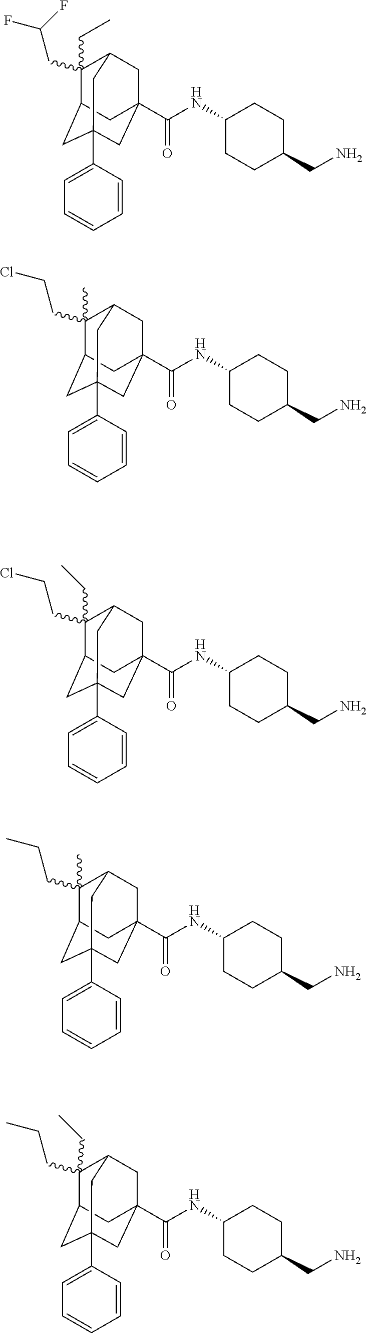

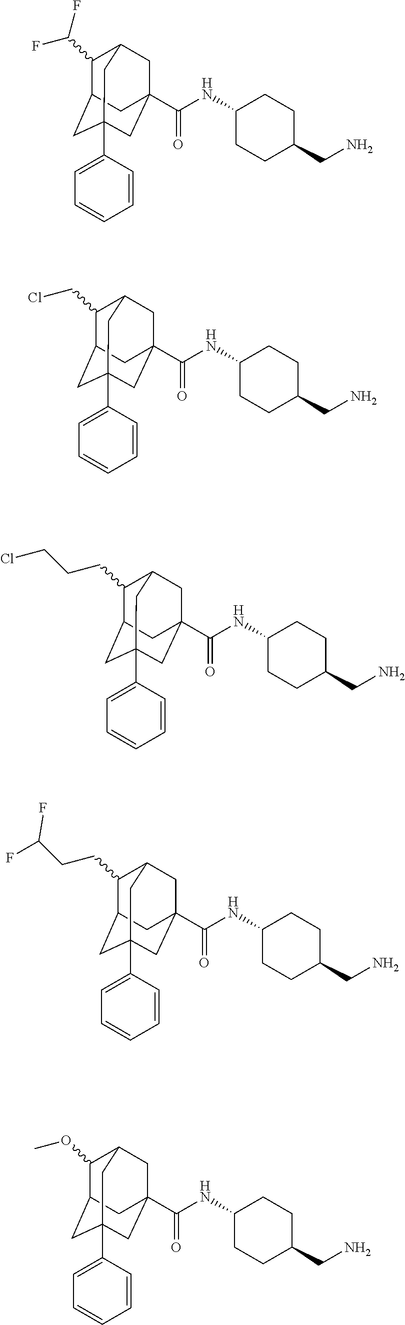 [SMILES: NC[C@H]1CC[C@H](NC(=O)C23CC4CC(c5ccccc5)(CC(C2)C4C(F)F)C3)CC1 ; NC[C@H]1CC[C@H](NC(=O)C23CC4CC(c5ccccc5)(CC(C2)C4CCl)C3)CC1 ; NC[C@H]1CC[C@H](NC(=O)C23CC4CC(c5ccccc5)(CC(C2)C4CCCCl)C3)CC1 ; NC[C@H]1CC[C@H](NC(=O)C23CC4CC(c5ccccc5)(CC(C2)C4CCC(F)F)C3)CC1 ; COC1C2CC3(C(=O)N[C@H]4CC[C@H](CN)CC4)CC1CC(c1ccccc1)(C2)C3]

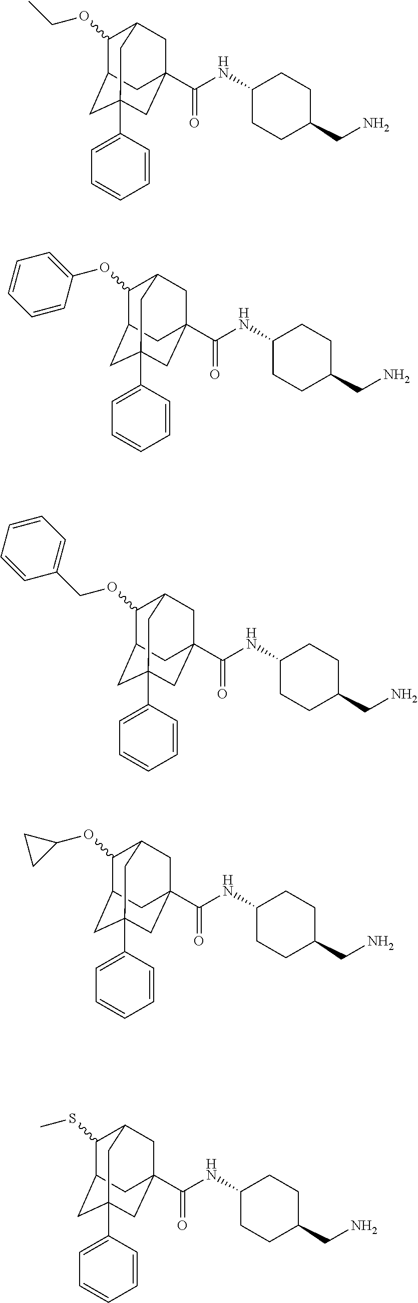 [SMILES: CCOC1C2CC3(C(=O)N[C@H]4CC[C@H](CN)CC4)CC1CC(c1ccccc1)(C2)C3 ; NC[C@H]1CC[C@H](NC(=O)C23CC4CC(c5ccccc5)(CC(C2)C4Oc2ccccc2)C3)CC1 ; NC[C@H]1CC[C@H](NC(=O)C23CC4CC(c5ccccc5)(CC(C2)C4OCc2ccccc2)C3)CC1 ; NC[C@H]1CC[C@H](NC(=O)C23CC4CC(c5ccccc5)(CC(C2)C4OC2CC2)C3)CC1 ; CSC1C2CC3(C(=O)N[C@H]4CC[C@H](CN)CC4)CC1CC(c1ccccc1)(C2)C3]

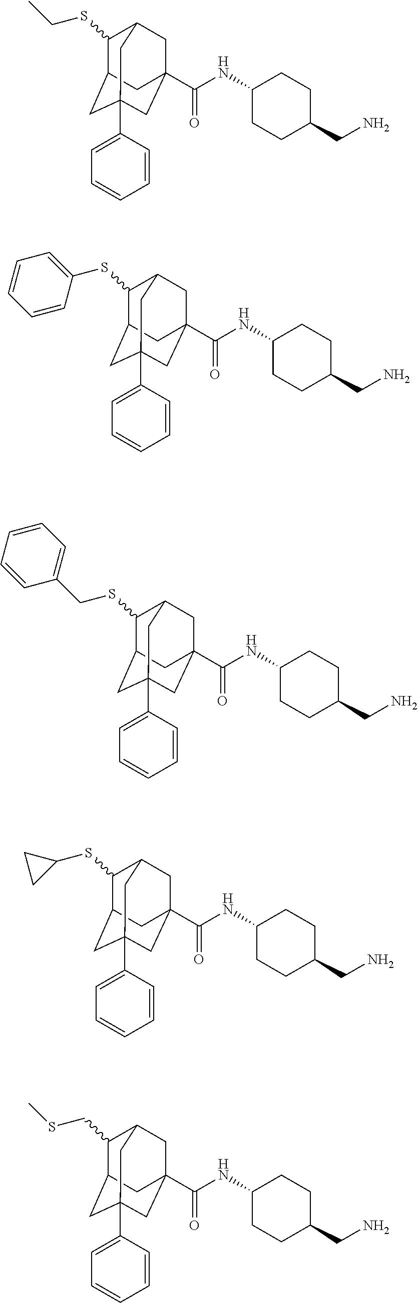 [SMILES: CCSC1C2CC3(C(=O)N[C@H]4CC[C@H](CN)CC4)CC1CC(c1ccccc1)(C2)C3 ; NC[C@H]1CC[C@H](NC(=O)C23CC4CC(c5ccccc5)(CC(C2)C4Sc2ccccc2)C3)CC1 ; NC[C@H]1CC[C@H](NC(=O)C23CC4CC(c5ccccc5)(CC(C2)C4SCc2ccccc2)C3)CC1 ; NC[C@H]1CC[C@H](NC(=O)C23CC4CC(c5ccccc5)(CC(C2)C4SC2CC2)C3)CC1 ; CSCC1C2CC3(C(=O)N[C@H]4CC[C@H](CN)CC4)CC1CC(c1ccccc1)(C2)C3]

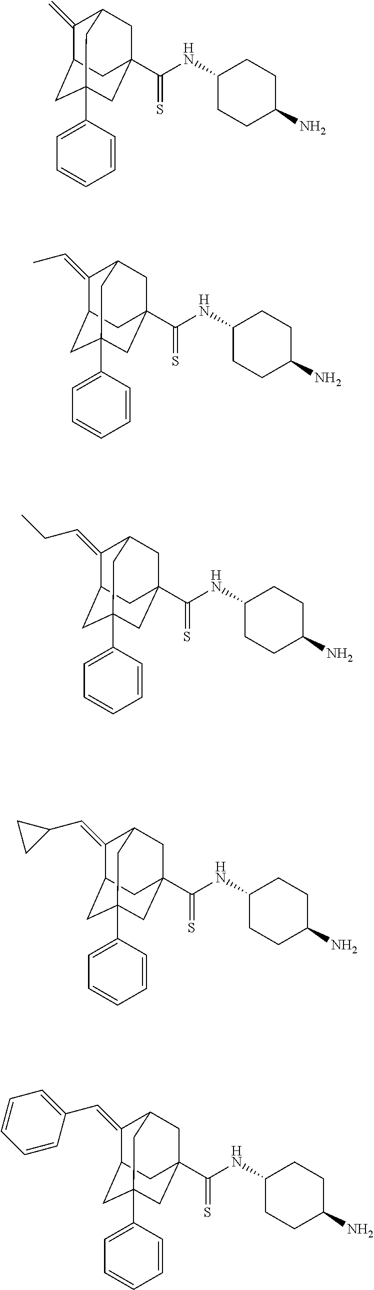 [SMILES: C=C1C2CC3(C(=S)N[C@H]4CC[C@H](N)CC4)CC1CC(c1ccccc1)(C2)C3 ; CC=C1C2CC3(C(=S)N[C@H]4CC[C@H](N)CC4)CC1CC(c1ccccc1)(C2)C3 ; CCC=C1C2CC3(C(=S)N[C@H]4CC[C@H](N)CC4)CC1CC(c1ccccc1)(C2)C3 ; N[C@H]1CC[C@H](NC(=S)C23CC4CC(c5ccccc5)(CC(C2)C4=CC2CC2)C3)CC1 ; N[C@H]1CC[C@H](NC(=S)C23CC4CC(c5ccccc5)(CC(C2)C4=Cc2ccccc2)C3)CC1]

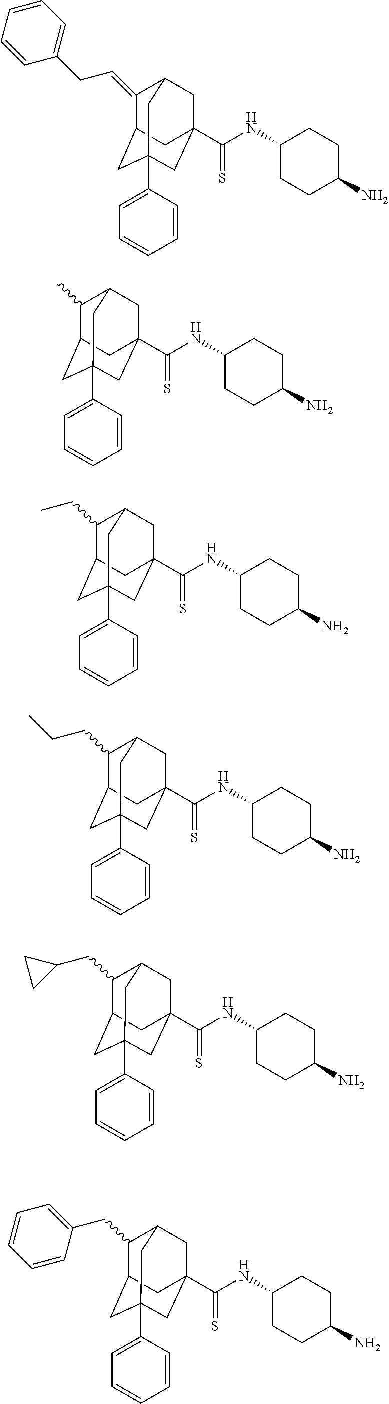

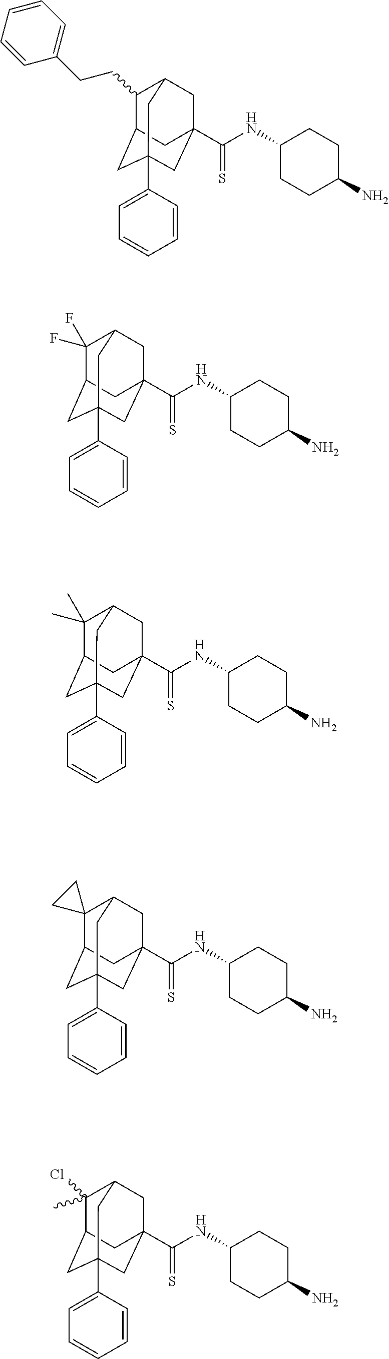

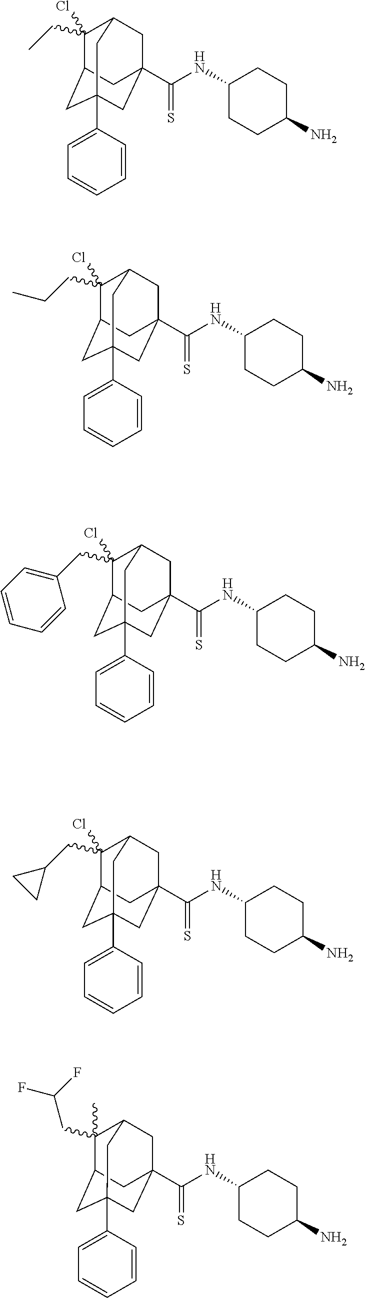

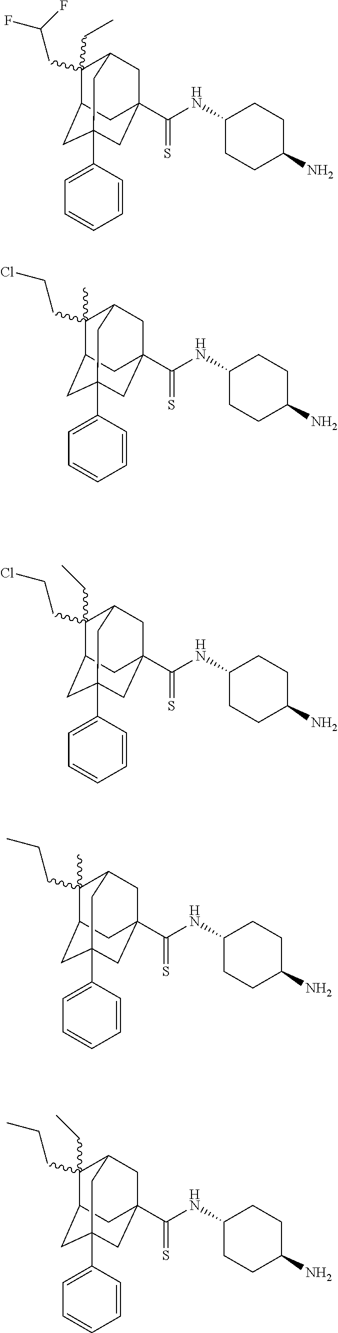

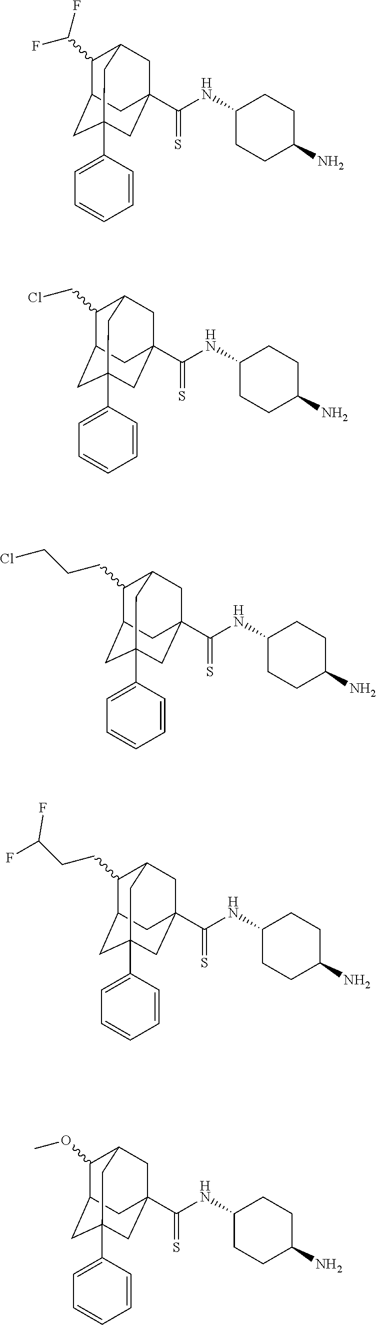 [SMILES: N[C@H]1CC[C@H](NC(=S)C23CC4CC(c5ccccc5)(CC(C2)C4C(F)F)C3)CC1 ; N[C@H]1CC[C@H](NC(=S)C23CC4CC(c5ccccc5)(CC(C2)C4CCl)C3)CC1 ; N[C@H]1CC[C@H](NC(=S)C23CC4CC(c5ccccc5)(CC(C2)C4CCCCl)C3)CC1 ; N[C@H]1CC[C@H](NC(=S)C23CC4CC(c5ccccc5)(CC(C2)C4CCC(F)F)C3)CC1 ; COC1C2CC3(C(=S)N[C@H]4CC[C@H](N)CC4)CC1CC(c1ccccc1)(C2)C3]

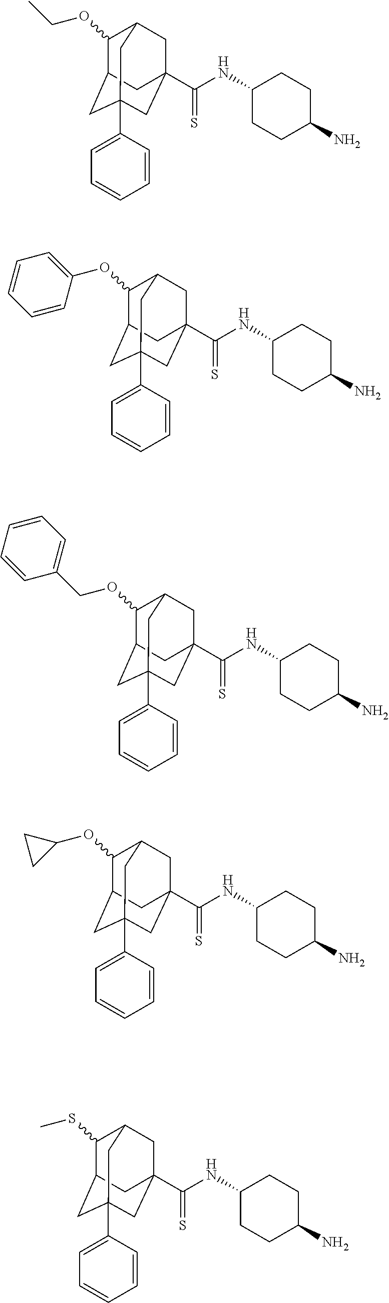 [SMILES: CCOC1C2CC3(C(=S)N[C@H]4CC[C@H](N)CC4)CC1CC(c1ccccc1)(C2)C3 ; N[C@H]1CC[C@H](NC(=S)C23CC4CC(c5ccccc5)(CC(C2)C4Oc2ccccc2)C3)CC1 ; N[C@H]1CC[C@H](NC(=S)C23CC4CC(c5ccccc5)(CC(C2)C4OCc2ccccc2)C3)CC1 ; N[C@H]1CC[C@H](NC(=S)C23CC4CC(c5ccccc5)(CC(C2)C4OC2CC2)C3)CC1 ; CSC1C2CC3(C(=S)N[C@H]4CC[C@H](N)CC4)CC1CC(c1ccccc1)(C2)C3]

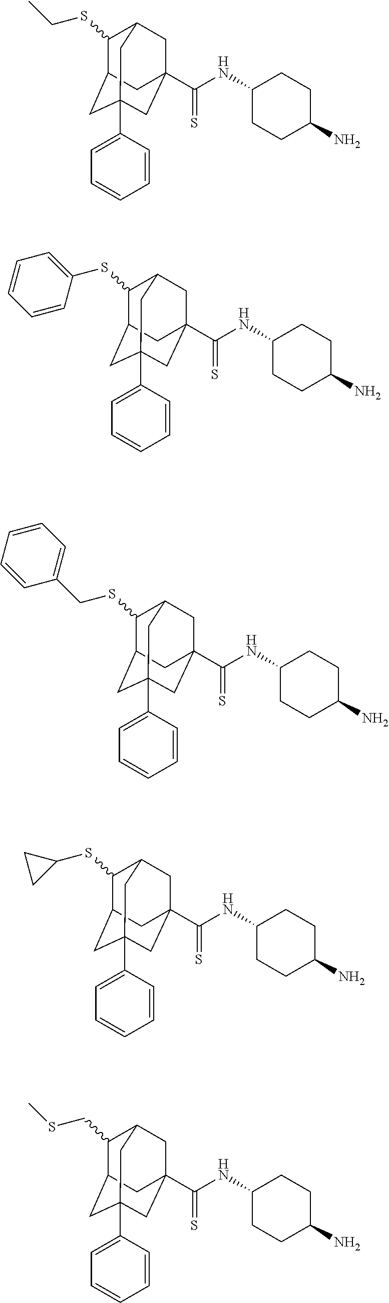 [SMILES: CCSC1C2CC3(C(=S)N[C@H]4CC[C@H](N)CC4)CC1CC(c1ccccc1)(C2)C3 ; N[C@H]1CC[C@H](NC(=S)C23CC4CC(c5ccccc5)(CC(C2)C4Sc2ccccc2)C3)CC1 ; N[C@H]1CC[C@H](NC(=S)C23CC4CC(c5ccccc5)(CC(C2)C4SCc2ccccc2)C3)CC1 ; N[C@H]1CC[C@H](NC(=S)C23CC4CC(c5ccccc5)(CC(C2)C4SC2CC2)C3)CC1 ; CSCC1C2CC3(C(=S)N[C@H]4CC[C@H](N)CC4)CC1CC(c1ccccc1)(C2)C3]

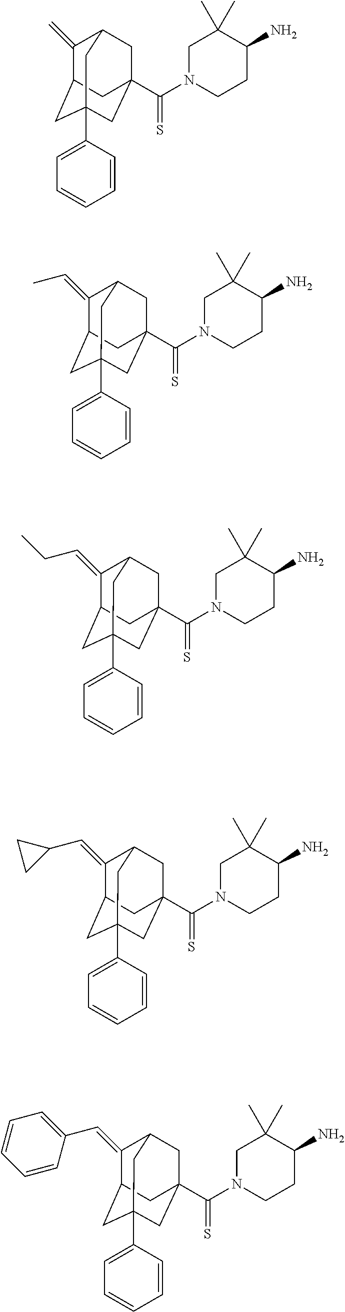 [SMILES: C=C1C2CC3(C(=S)N4CC[C@H](N)C(C)(C)C4)CC1CC(c1ccccc1)(C2)C3 ; CC=C1C2CC3(C(=S)N4CC[C@H](N)C(C)(C)C4)CC1CC(c1ccccc1)(C2)C3 ; CCC=C1C2CC3(C(=S)N4CC[C@H](N)C(C)(C)C4)CC1CC(c1ccccc1)(C2)C3 ; CC1(C)CN(C(=S)C23CC4CC(c5ccccc5)(CC(C2)C4=CC2CC2)C3)CC[C@@H]1N ; CC1(C)CN(C(=S)C23CC4CC(c5ccccc5)(CC(C2)C4=Cc2ccccc2)C3)CC[C@@H]1N]

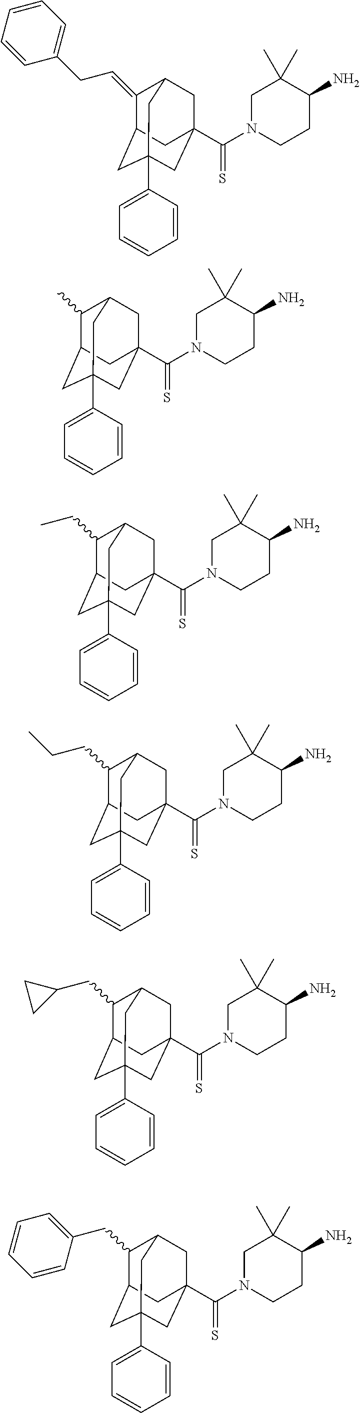

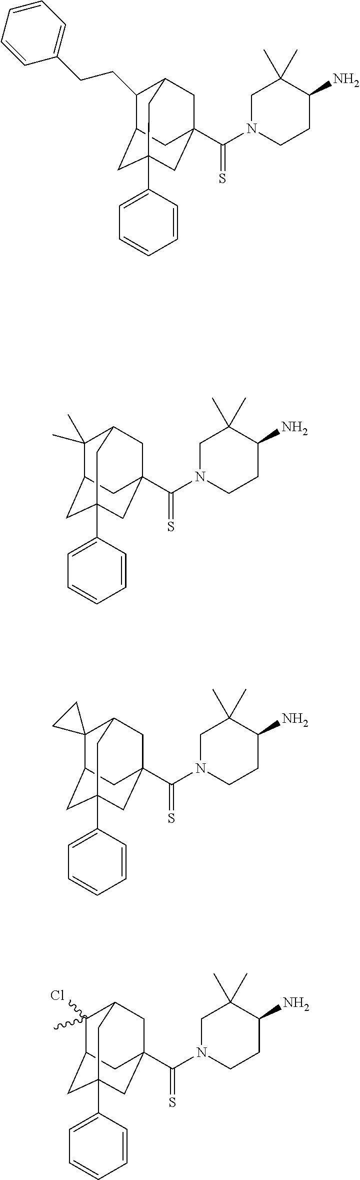

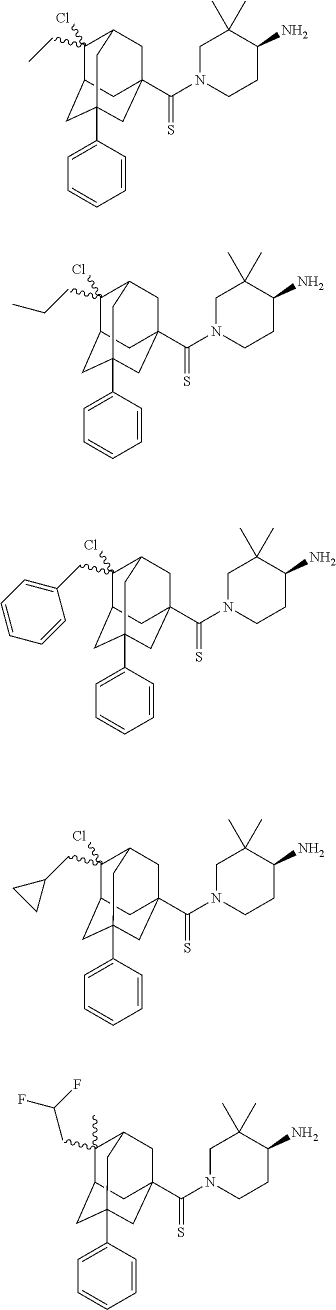

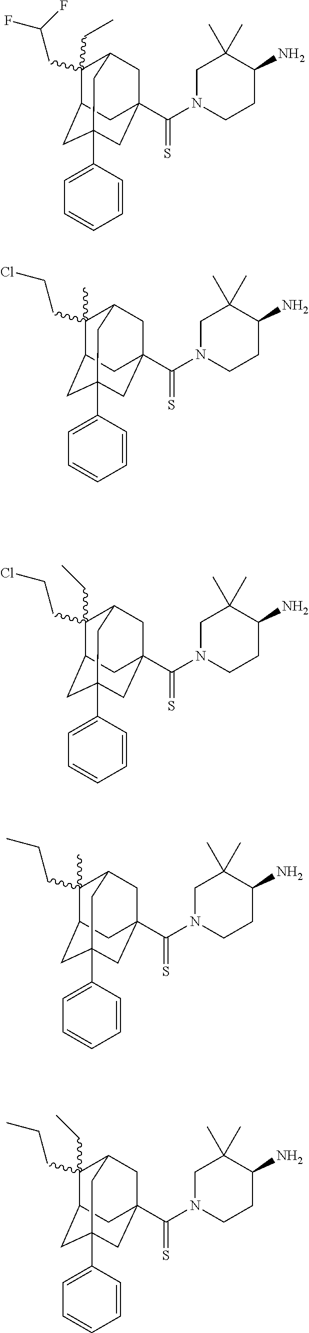

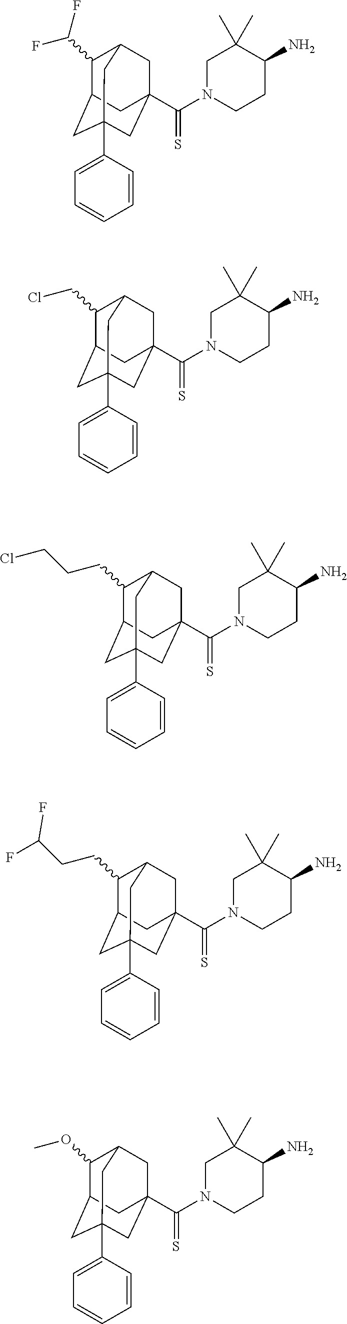 [SMILES: CC1(C)CN(C(=S)C23CC4CC(c5ccccc5)(CC(C2)C4C(F)F)C3)CC[C@@H]1N ; CC1(C)CN(C(=S)C23CC4CC(c5ccccc5)(CC(C2)C4CCl)C3)CC[C@@H]1N ; CC1(C)CN(C(=S)C23CC4CC(c5ccccc5)(CC(C2)C4CCCCl)C3)CC[C@@H]1N ; CC1(C)CN(C(=S)C23CC4CC(c5ccccc5)(CC(C2)C4CCC(F)F)C3)CC[C@@H]1N ; COC1C2CC3(C(=S)N4CC[C@H](N)C(C)(C)C4)CC1CC(c1ccccc1)(C2)C3]

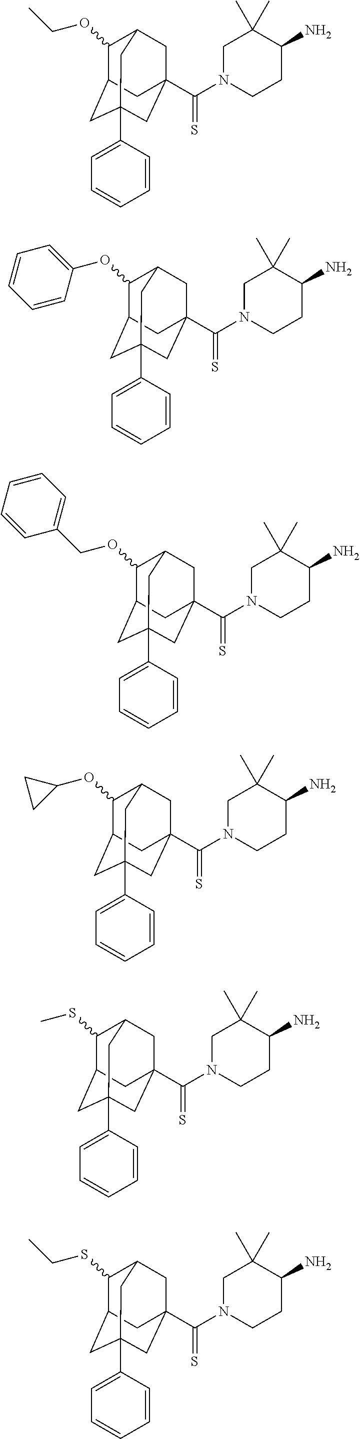 [SMILES: CCOC1C2CC3(C(=S)N4CC[C@H](N)C(C)(C)C4)CC1CC(c1ccccc1)(C2)C3 ; CC1(C)CN(C(=S)C23CC4CC(c5ccccc5)(CC(C2)C4Oc2ccccc2)C3)CC[C@@H]1N ; CC1(C)CN(C(=S)C23CC4CC(c5ccccc5)(CC(C2)C4OCc2ccccc2)C3)CC[C@@H]1N ; CC1(C)CN(C(=S)C23CC4CC(c5ccccc5)(CC(C2)C4OC2CC2)C3)CC[C@@H]1N ; CSC1C2CC3(C(=S)N4CC[C@H](N)C(C)(C)C4)CC1CC(c1ccccc1)(C2)C3 ; CCSC1C2CC3(C(=S)N4CC[C@H](N)C(C)(C)C4)CC1CC(c1ccccc1)(C2)C3]

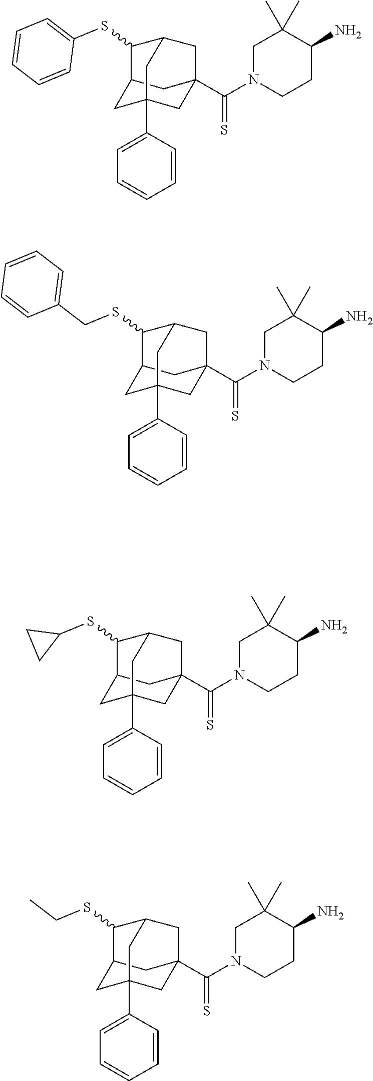 [SMILES: CC1(C)CN(C(=S)C23CC4CC(c5ccccc5)(CC(C2)C4Sc2ccccc2)C3)CC[C@@H]1N ; CC1(C)CN(C(=S)C23CC4CC(c5ccccc5)(CC(C2)C4SCc2ccccc2)C3)CC[C@@H]1N ; CC1(C)CN(C(=S)C23CC4CC(c5ccccc5)(CC(C2)C4SC2CC2)C3)CC[C@@H]1N ; CCSC1C2CC3(C(=S)N4CC[C@H](N)C(C)(C)C4)CC1CC(c1ccccc1)(C2)C3]

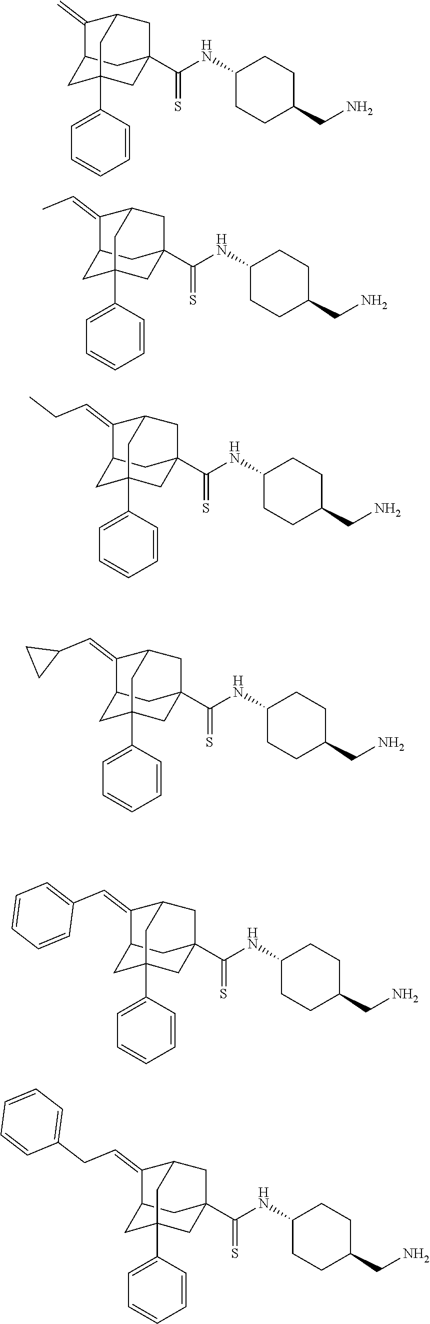

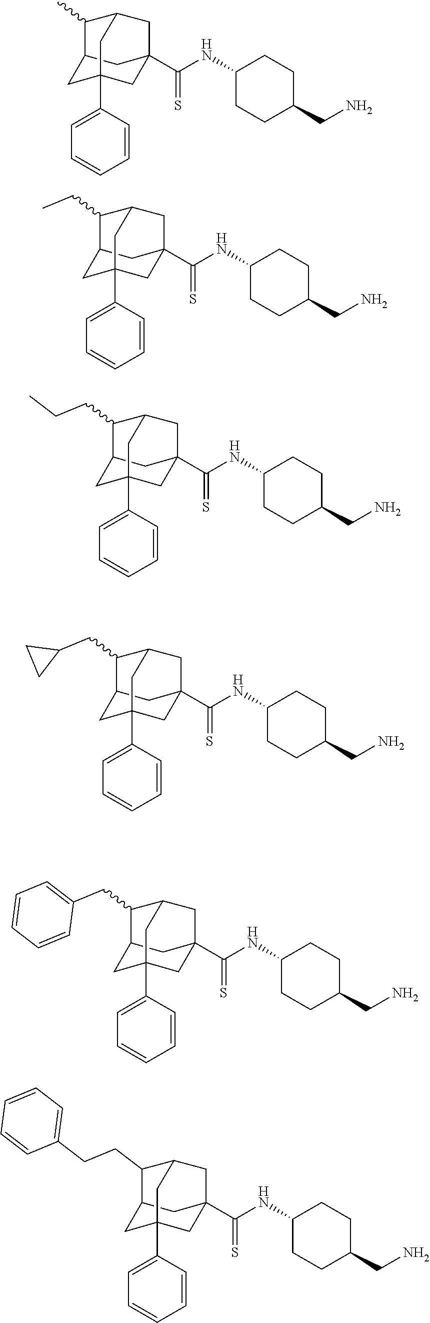

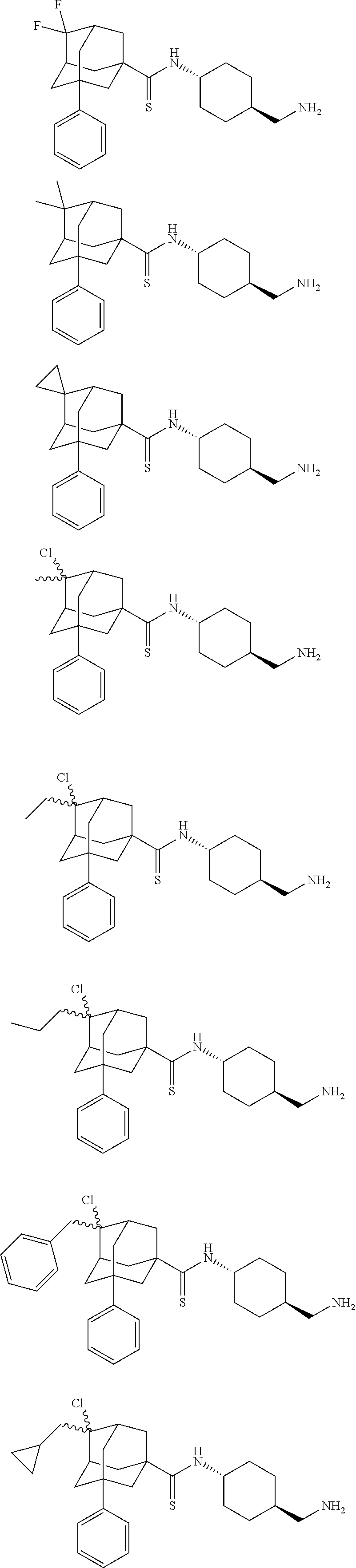

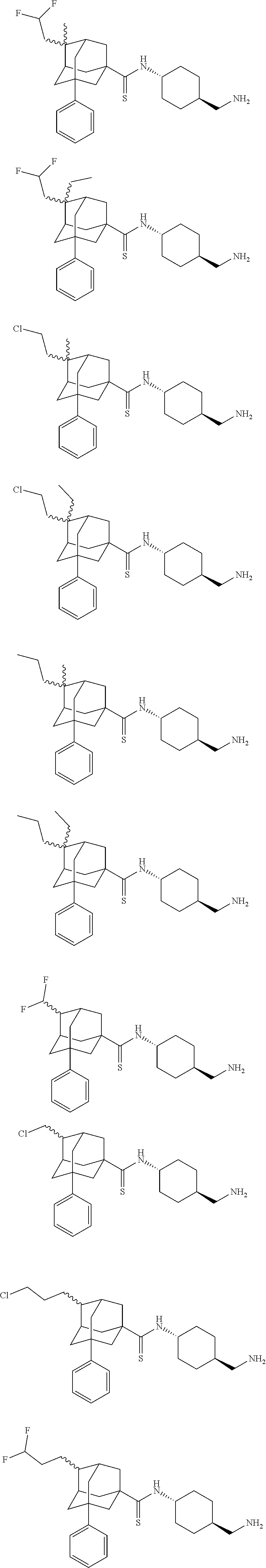

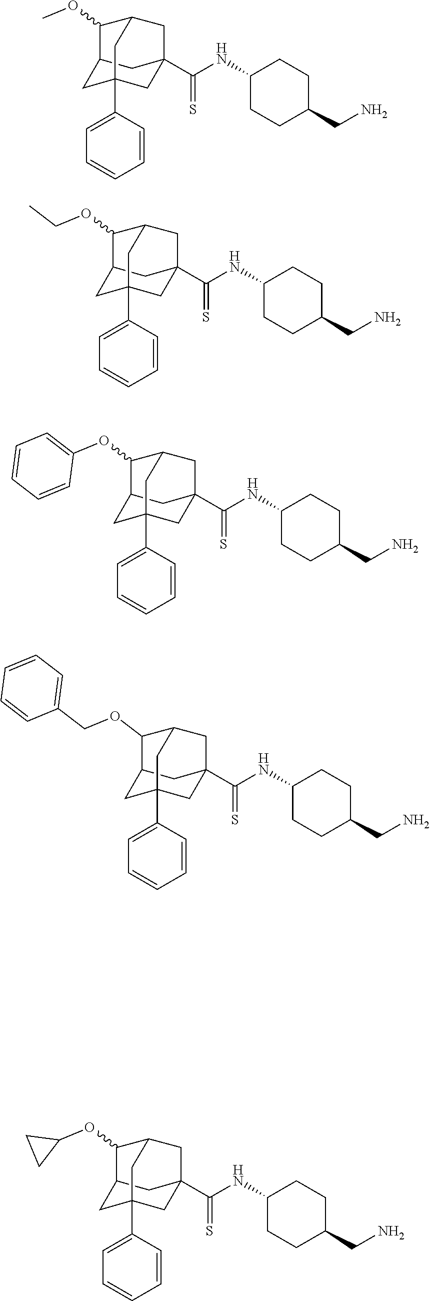

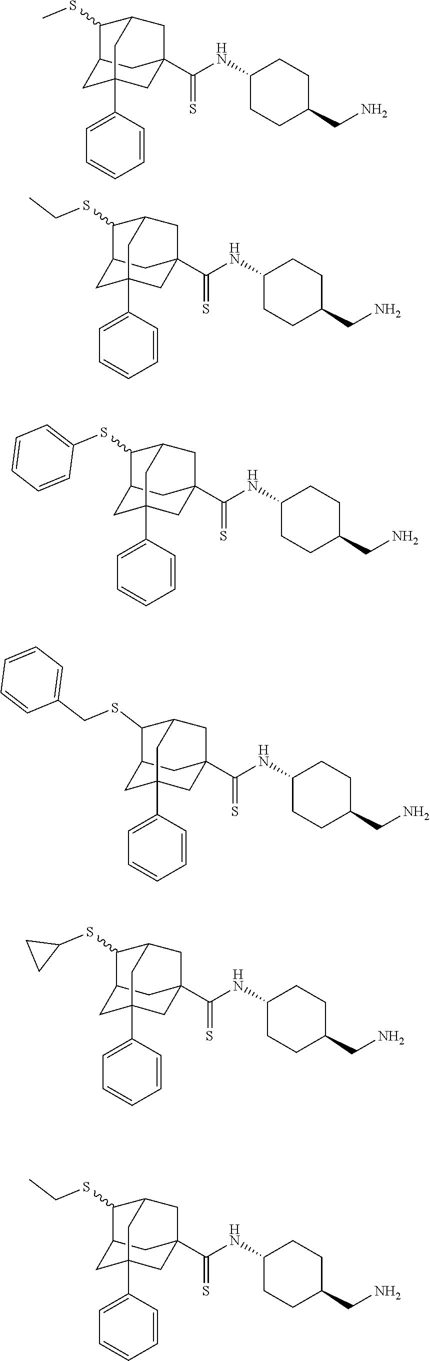

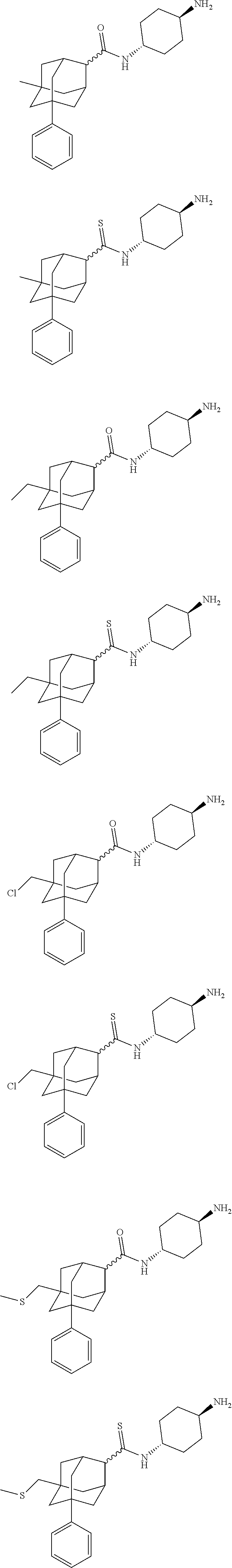

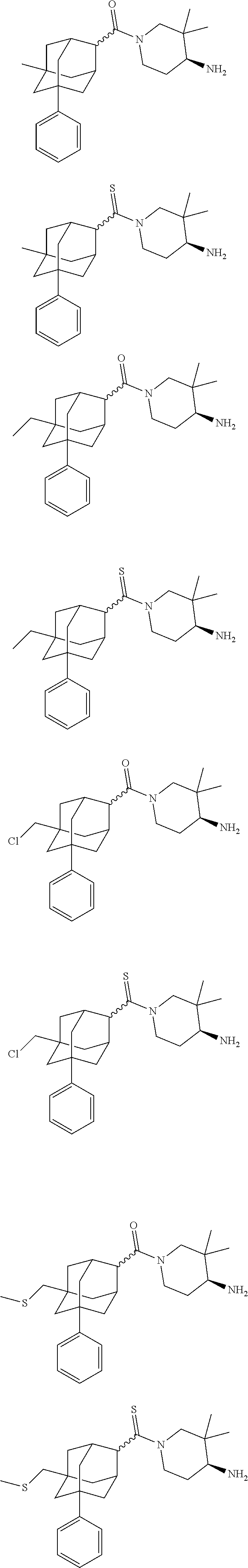

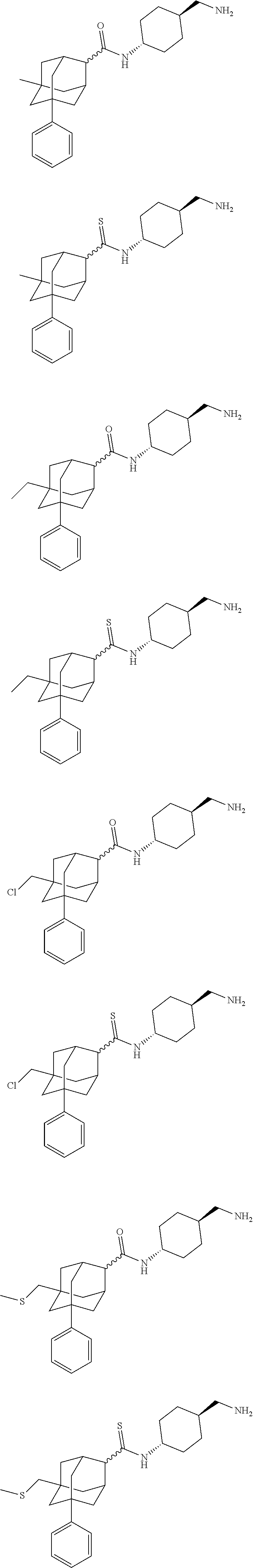

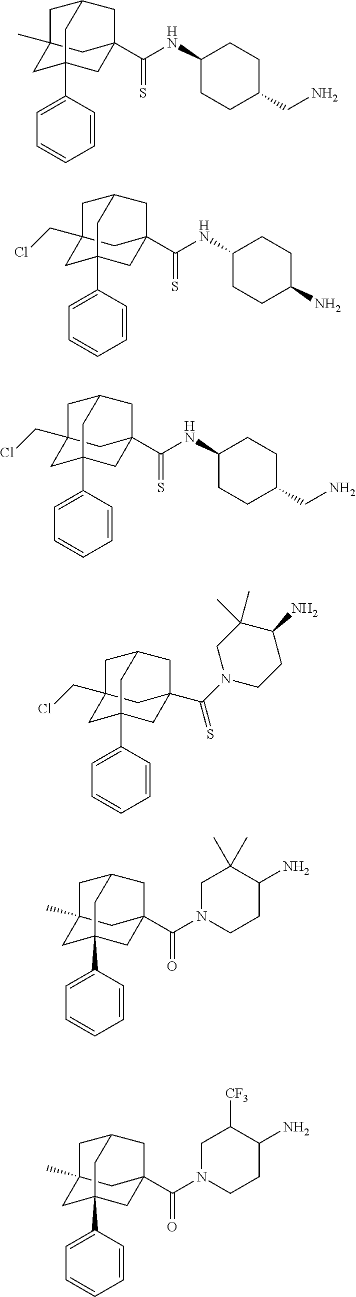

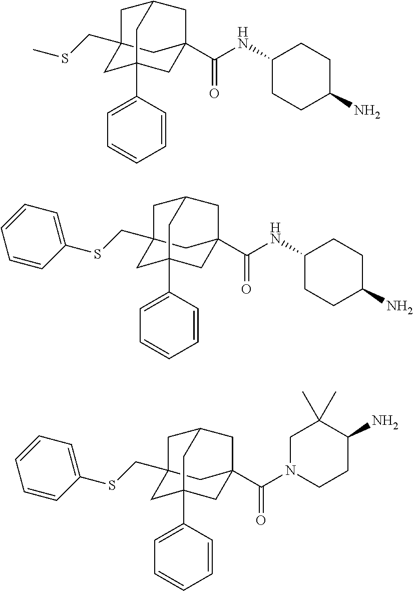

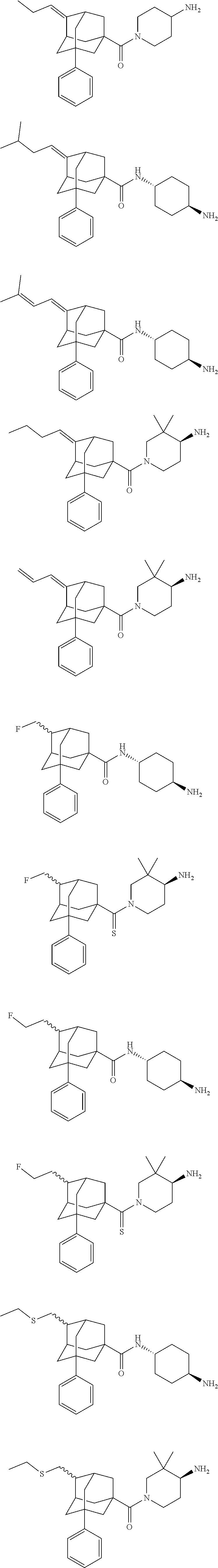

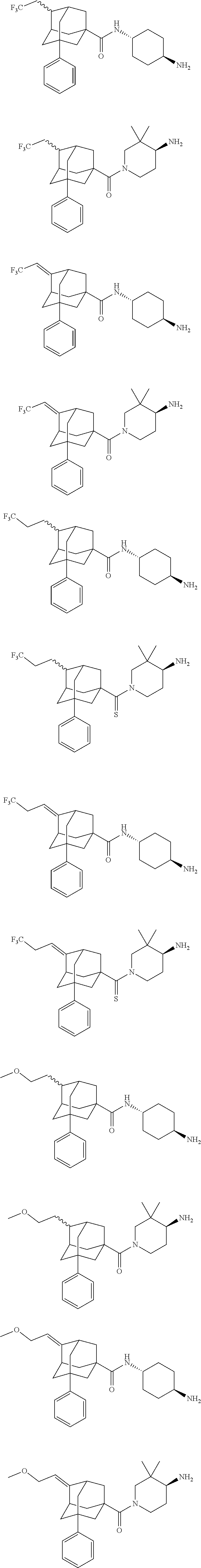

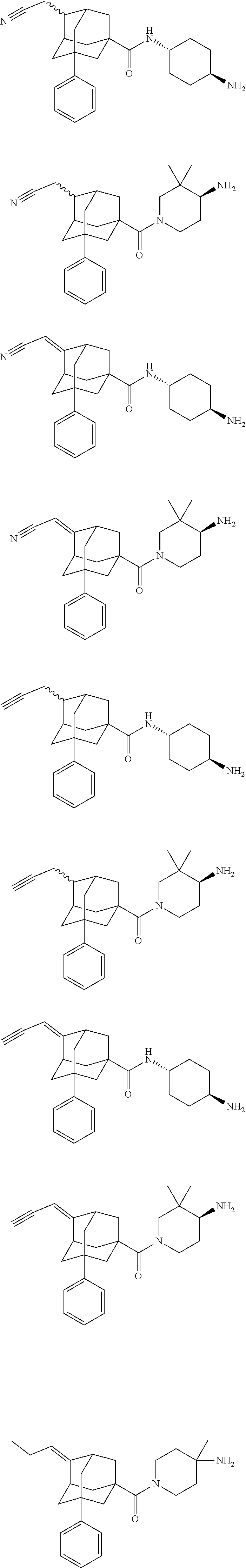

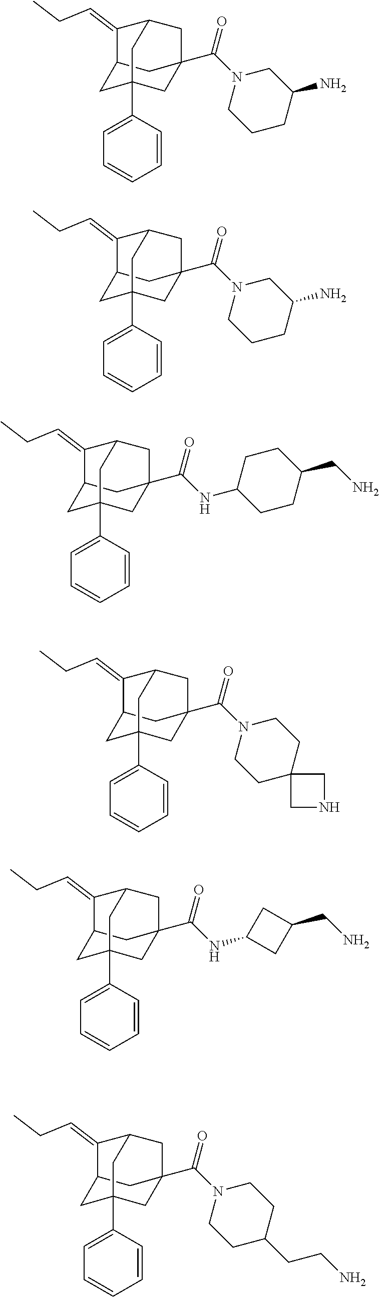

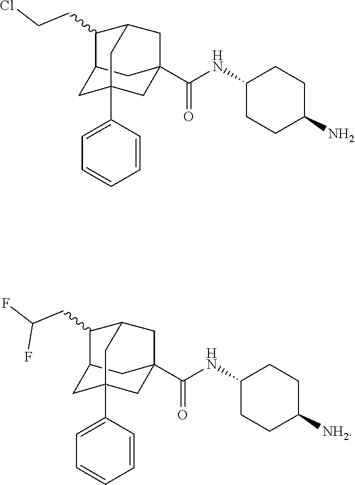 [SMILES: N[C@H]1CC[C@H](NC(=O)C23CC4CC(c5ccccc5)(CC(C2)C4CCCl)C3)CC1 ; N[C@H]1CC[C@H](NC(=O)C23CC4CC(c5ccccc5)(CC(C2)C4CC(F)F)C3)CC1]

43. The compound of claim 42, selected from the group consisting of: 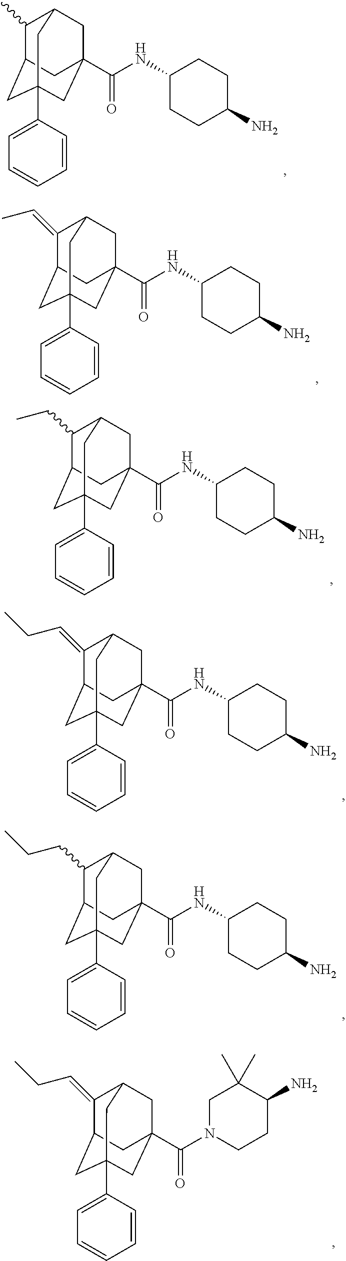 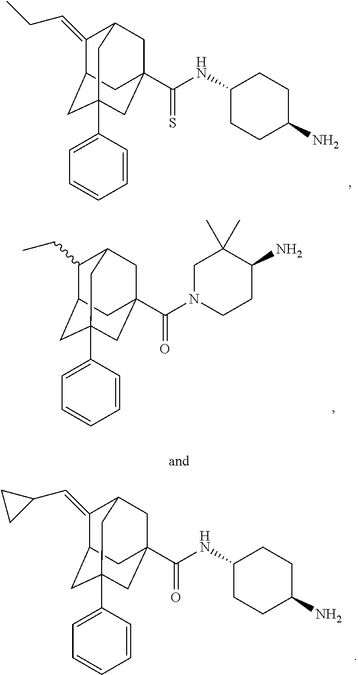 [SMILES: CCC=C1C2CC3(C(=S)N[C@H]4CC[C@H](N)CC4)CC1CC(c1ccccc1)(C2)C3 ; CCC1C2CC3(C(=O)N4CC[C@H](N)C(C)(C)C4)CC1CC(c1ccccc1)(C2)C3 ; N[C@H]1CC[C@H](NC(=O)C23CC4CC(c5ccccc5)(CC(C2)C4=CC2CC2)C3)CC1]
